(19) United States
(12) Patent Application Publication
(10) Pub. No.: US 2026/0116845 A1
(43) Pub. Date: Apr. 30, 2026

(54) LIPIDS AND COMPOSITIONS THEREOF

(71) Applicant: Kernal Biologics, Inc., Boston, MA (US)

(72) Inventors: Ryan F. LANDIS, Boston, MA (US); Balkrishen BHAT, Boston, MA (US); Jieni XU, Boston, MA (US); Cafer OZDEMIR, Boston, MA (US); Burak YILMAZ, Boston, MA (US); Yusuf ERKUL, Boston, MA (US); Manfred KRAUS, Boston, MA (US)

(21) Appl. No.: 19/434,722

(22) Filed: Dec. 29, 2025

Related U.S. Application Data
(63) Continuation of application No. PCT/US2024/036593, filed on Jul. 2, 2024.
(60) Provisional application No. 63/511,824, filed on Jul. 3, 2023.

(51) Int. Cl. C07C 229/16; A61K 9/51; A61K 48/00; C07B 59/00; C07C 229/12; C07D 265/30

(52) U.S. Cl. CPC C07C 229/16; A61K 9/5123; A61K 48/0033; C07B 59/001; C07C 229/12; C07D 265/30; C07B 2200/05

(57) ABSTRACT

The present disclosure relates to cationic and/or ionizable lipids and nucleic acid-lipid particle compositions comprising the same. The present disclosure also relates to methods of using and delivering the described lipids and lipid-containing particles.

### CROSS-REFERENCE

This application is a continuation of International Application No. PCT/US2024/036593, filed Jul. 2, 2024, which claims the benefit of U.S. Provisional Application No. 63/511,824, filed Jul. 3, 2023, the entirety of each of which is hereby incorporated by reference.

### BACKGROUND

Lipids are amphiphilic molecules that contain three domains: a polar head group, a hydrophobic tail region and a linker between the two domains. Lipid-containing particles have been used as transport vehicles for therapeutic agents such as nucleic acids, small molecules compounds, and proteins into cells and other intracellular compartments. Cationic lipids, ionizable lipids and other types of lipid have been explored for mRNA delivery.

### SUMMARY

Although a variety of lipid-containing particle compositions have been demonstrated, there remains a need to improve the safety, efficacy, and specificity of such nanoparticle-based transport vehicles. In some embodiments, a lipid-containing particle compositions as described herein demonstrate improved delivery efficiency. In some embodiments, a lipid-containing particle compositions as described herein demonstrate improved toxicity profiles. In some embodiments, a lipid-containing particle compositions as described herein demonstrate improved tolerability (e.g., liver tolerability). In some embodiments, a lipid-containing particle compositions as described herein comprises a lipid as described herein. Without wishing to be bound by any particular theory, the present disclosure provides an insight that certain modifications on the lipid structure demonstrate improved tolerability (e.g., liver tolerability) while preserving delivery performance. In some embodiments, a structure modification comprises halogenation of an aliphatic chain of a lipid. In some embodiments, a lipid described herein comprises halogen. In some embodiments, a lipid described herein comprises F. Without wishing to be bound by any particular theory, in some embodiments, the present disclosure provides an insight that a lipid comprising halogen (e.g., F) surprisingly demonstrates improved tolerability over an otherwise identical lipid that does not comprise halogen (e.g., F). Described herein, in some aspects, is a compound of Formula I′:

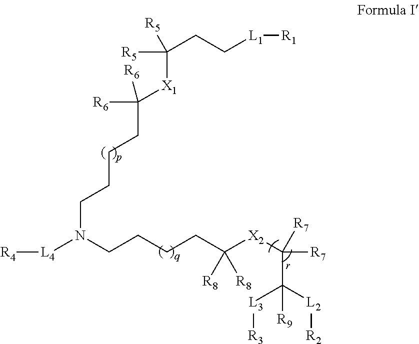

or its N-oxide, or a pharmaceutically acceptable salt thereof,
wherein:
L₁ is substituted or unsubstituted linear —C₃₋₁₂ alkylene or —C₃₋₁₂ alkenylalkylene;
L₂ is substituted or unsubstituted linear —C₄₋₁₂ alkylene or —C₃₋₁₂ alkenylalkylene;
L₃ is substituted or unsubstituted linear —C₄₋₁₂ alkylene or —C₃₋₁₂ alkenylalkylene;
wherein if L₁, L₂, or L₃ is substituted, then L₁, L₂, or L₃ is substituted with 1-5 substituents selected from the group consisting of halogen, deuterium, —CN, linear or branched C₁₋₁₀ alkyl, linear or branched C₁₋₁₀ heteroalkyl, C₁₋₂ haloalkyl, —C(═O)R¹⁰, —C(═O)N(R¹⁰)₂, —OR¹⁰, —N(R¹⁰)₂, substituted or unsubstituted C₃₋₆ cycloalkyl, and substituted or unsubstituted 3- to 10-membered heterocycloalkyl; wherein each substituted C₃₋₆ cycloalkyl and substituted 3- to 10-membered heterocycloalkyl is substituted with 1-5 R¹¹;
L₄ is substituted or unsubstituted —C₁₋₂₄ alkylene or —C₃₋₂₄ alkenylalkylene; wherein L₄ is optionally substituted with 1 to 10 R¹¹;
X₁ is a covalent bond, —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—;
X₂ is a covalent bond, —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—;
each R₁, R₂, and R₃, is independently hydrogen, deuterium, halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl;
provided that at least one of R₁, R₂, and R₃ is not hydrogen or deuterium;
or when each R₁, R₂, and R₃ is hydrogen, or deuterium, then at least one of L₁, L₂, L₃ is substituted with 1-5 substituents, and at least one of the substituents is not a linear C₁₋₁₀ alkyl substituted at a terminal carbon, or a deuterium;
R₄ is halogen, —OH, —OR¹⁰, —O—C(═O)—R¹⁰, —C(═O)—OR¹⁰, —O—C(═O)—OR¹⁰—, —C(═O)—O—C(═O)—R¹⁰, —O—C(═O)—C₀₋₉ alkylene-R¹¹, —(O—C₀₋₉ alkylene-)ₙR¹¹, —O—C(═O)—C₀₋₉ alkylene-N(R¹⁰)₂, —N(R¹⁰)₂, or —C(═O)—C₀₋₉ alkylene-N(R¹⁰)₂;
each R₅, R₆, R₇, R₈, or R₉ is independently hydrogen, deuterium, halogen, —CN, —OR¹⁰, —N(R¹⁰)₂, —O—C(═O)—R¹⁰, C₁₋₁₀ alkyl, C₁₋₁₀ heteroalkyl, C₃₋₁₀ cycloalkyl, C₃₋₁₀ heterocycloalkyl, C₂₋₆ alkenyl, or C₂₋₆ alkynyl;
each R¹⁰ is independently hydrogen, C₁₋₁₀ alkyl, C₁₋₁₀ heteroalkyl, C₁₋₁₀ haloalkyl, C₃₋₁₀ cycloalkyl, C₃₋₁₀ heterocycloalkyl, C₂₋₆ alkenyl, or C₂₋₆ alkynyl;
each R¹¹ is independently hydrogen, deuterium, halogen, —CN, C₁₋₁₀ alkyl, C₁₋₁₀ heteroalkyl, C₁₋₁₀ haloalkyl, C₃₋₁₀ cycloalkyl, C₃₋₁₀ heterocycloalkyl, C₂₋₆ alkenyl, C₂₋₆ alkynyl, —C(O)OH, —C(O)—O—C₁₋₆ alkyl, —C(O)—C₁₋₆ alkyl, —C(O)NH₂, —C(O)NH(C₁₋₆ alkyl), —C(O)N(C₁₋₆ alkyl)₂, —NH₂, —NH(C₁₋₆ alkyl), —N(C₁₋₆ alkyl)₂, —OH, —O—C₁₋₆ alkyl, —SH, —S(C₁₋₆ alkyl), —S(O)(C₁₋₆ alkyl), —S(O)₂(C₁₋₆ alkyl), or —S(O)₂NH(C₁₋₆ alkyl);
n is an integer from 0-20;
p is 0, 1, 2, 3, 4, or 5;
q is 0, 1, 2, 3, 4, or 5; and
r is 0 or 1.

In some aspects, r is 0. In some aspects, described herein is a compound of Formula I′-a:

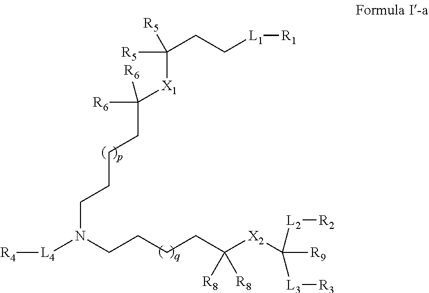

wherein:
L₁ is substituted or unsubstituted linear —C₃₋₁₂ alkylene or —C₃₋₁₂ alkenylalkylene;
L₂ is substituted or unsubstituted linear —C₄₋₁₂ alkylene or —C₃₋₁₂ alkenylalkylene;
L₃ is substituted or unsubstituted linear —C₄₋₁₂ alkylene or —C₃₋₁₂ alkenylalkylene;
wherein if L₁, L₂, or L₃ is substituted, then L₁, L₂, or L₃ is substituted with 1-5 substituents selected from the group consisting of halogen, deuterium, —CN, linear or branched C₁₋₁₀ alkyl, linear or branched C₁₋₁₀ heteroalkyl, C₁₋₂ haloalkyl, —C(═O)R¹⁰, —C(═O)N(R¹⁰)₂, —OR¹⁰, —N(R¹⁰)₂, substituted or unsubstituted C₃₋₆ cycloalkyl, and substituted or unsubstituted 3- to 10-membered heterocycloalkyl; wherein each substituted C₃₋₆ cycloalkyl and substituted 3- to 10-membered heterocycloalkyl is substituted with 1-5 R¹¹;
L₄ is substituted or unsubstituted —C₁₋₂₄ alkylene or —C₃₋₂₄ alkenylalkylene; wherein L₄ is optionally substituted with 1 to 10 R¹¹;
X₁ is a covalent bond, —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—;
X₂ is a covalent bond, —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—;
each R₁, R₂, and R₃, is independently hydrogen, deuterium, halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl;
provided that at least one of R₁, R₂, and R₃ is not hydrogen or deuterium;
or when each R₁, R₂, and R₃ is hydrogen, or deuterium, then at least one of L₁, L₂, L₃ is substituted with 1-5 substituents, and at least one of the substituents is not a linear C₁₋₁₀ alkyl substituted at a terminal carbon, or a deuterium;
R₄ is halogen, —OH, —OR¹⁰, —O—C(═O)—R¹⁰, —C(═O)—OR¹⁰, —O—C(═O)—OR¹⁰—, —C(═O)—O—C(═O)—R¹⁰, —O—C(═O)—C₀₋₉ alkylene-R¹¹, —(O—C₀₋₉ alkylene-)ₙR¹¹, —O—C(═O)—C₀₋₉ alkylene-N(R¹⁰)₂, —N(R¹⁰)₂, or —C(═O)—C₀₋₉ alkylene-N(R¹⁰)₂;
each R₅, R₆, R₈, or R₉ is independently hydrogen, deuterium, halogen, —CN, —OR¹⁰, —N(R¹⁰)₂, —O—C(═O)—R¹⁰, C₁₋₁₀ alkyl, C₁₋₁₀ heteroalkyl, C₃₋₁₀ cycloalkyl, C₃₋₁₀ heterocycloalkyl, C₂₋₆ alkenyl, or C₂₋₆ alkynyl;
each R¹⁰ is independently hydrogen, C₁₋₁₀ alkyl, C₁₋₁₀ heteroalkyl, C₁₋₁₀ haloalkyl, C₃₋₁₀ cycloalkyl, C₃₋₁₀ heterocycloalkyl, C₂₋₆ alkenyl, or C₂₋₆ alkynyl;
each R¹¹ is independently hydrogen, deuterium, halogen, —CN, C₁₋₁₀ alkyl, C₁₋₁₀ heteroalkyl, C₁₋₁₀ haloalkyl, C₃₋₁₀ cycloalkyl, C₃₋₁₀ heterocycloalkyl, C₂₋₆ alkenyl, C₂₋₆ alkynyl, —C(O)OH, —C(O)—O—C₁₋₆ alkyl, —C(O)—C₁₋₆ alkyl, —C(O)NH₂, —C(O)NH(C₁₋₆ alkyl), —C(O)N(C₁₋₆ alkyl)₂, —NH₂, —NH(C₁₋₆ alkyl), —N(C₁₋₆ alkyl)₂, —OH, —O—C₁₋₆ alkyl, —SH, —S(C₁₋₆ alkyl), —S(O)(C₁₋₆ alkyl), —S(O)₂(C₁₋₆ alkyl), or —S(O)₂NH(C₁₋₆ alkyl);
n is an integer from 0-20;
p is 0, 1, 2, 3, 4, or 5; and
q is 0, 1, 2, 3, 4, or 5.

In some aspects, r is 1. In some aspects, described herein is a compound of Formula I:

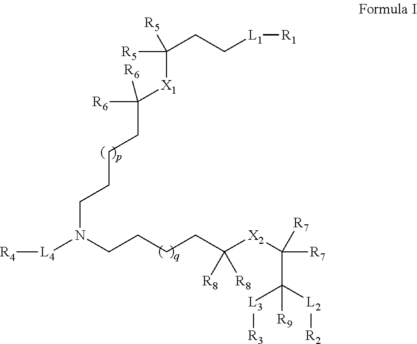

or its N-oxide, or a pharmaceutically acceptable salt thereof,
wherein:
L₁ is substituted or unsubstituted linear —C₃₋₁₂ alkylene or —C₃₋₁₂ alkenylalkylene;
L₂ is substituted or unsubstituted linear —C₄₋₁₂ alkylene or —C₃₋₁₂ alkenylalkylene;
L₃ is substituted or unsubstituted linear —C₄₋₁₂ alkylene or —C₃₋₁₂ alkenylalkylene;
wherein if L₁, L₂, or L₃ is substituted, then L₁, L₂, or L₃ is substituted with 1-5 substituents selected from the group consisting of halogen, deuterium, —CN, linear or branched C₁₋₁₀ alkyl, linear or branched C₁₋₁₀ heteroalkyl, C₁₋₂ haloalkyl, —C(═O)R¹⁰, —C(═O)N(R¹⁰)₂, —OR¹⁰, —N(R¹⁰)₂, substituted or unsubstituted C₃₋₆ cycloalkyl, and substituted or unsubstituted 3- to 10-membered heterocycloalkyl; wherein each substituted C₃₋₆ cycloalkyl and substituted 3- to 10-membered heterocycloalkyl is substituted with 1-5 R¹¹;
L₄ is substituted or unsubstituted —C₁₋₂₄ alkylene or —C₃₋₂₄ alkenylalkylene; wherein L₄ is optionally substituted with 1 to 10 R¹¹;
X₁ is —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—;
X₂ is —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—;
each R₁, R₂, and R₃, is independently hydrogen, deuterium, halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl;
provided that at least one of R₁, R₂, and R₃ is not hydrogen or deuterium;
or when each R₁, R₂, and R₃ is hydrogen, or deuterium, then at least one of L₁, L₂, L₃ is substituted with 1-5 substituents, and at least one of the substituents is not a linear C₁₋₁₀ alkyl substituted at a terminal carbon, or a deuterium;
R₄ is —OH, —OR¹⁰, —O—C(═O)—R¹⁰, —C(═O)—OR¹⁰, —O—C(═O)—OR¹⁰—, —C(═O)—O—C(═O)—R¹⁰, —O—C(═O)—C₀₋₉ alkylene-R¹¹, —(O—C₀₋₉ alkylene-)ₙR¹¹, —O—C(═O)—C₀₋₉ alkylene-N(R¹⁰)₂, —N(R¹⁰)₂, or —C(═O)—C₀₋₉ alkylene-N(R¹⁰)₂;
each R₅, R₆, R₇, R₈, or R₉ is independently hydrogen, deuterium, halogen, —CN, —OR¹⁰, —N(R¹⁰)₂, —O—C(═O)—R¹⁰, C₁₋₁₀ alkyl, C₁₋₁₀ heteroalkyl, C₃₋₁₀ cycloalkyl, C₃₋₁₀ heterocycloalkyl, C₂₋₆ alkenyl, or C₂₋₆ alkynyl;
each R¹⁰ is independently hydrogen, C₁₋₁₀ alkyl, C₁₋₁₀ heteroalkyl, C₁₋₁₀ haloalkyl, C₃₋₁₀ cycloalkyl, C₃₋₁₀ heterocycloalkyl, C₂₋₆ alkenyl, or C₂₋₆ alkynyl;
each R¹¹ is independently hydrogen, deuterium, halogen, —CN, C₁₋₁₀ alkyl, C₁₋₁₀ heteroalkyl, C₁₋₁₀ haloalkyl, C₃₋₁₀ cycloalkyl, C₃₋₁₀ heterocycloalkyl, C₂₋₆ alkenyl, C₂₋₆ alkynyl, —C(O)OH, —C(O)—O—C₁₋₆ alkyl, —C(O)—C₁₋₆ alkyl, —C(O)NH₂, —C(O)NH(C₁₋₆ alkyl), —C(O)N(C₁₋₆ alkyl)₂, —NH₂, —NH(C₁₋₆ alkyl), —N(C₁₋₆ alkyl)₂, —OH, —O—C₁₋₆ alkyl, —SH, —S(C₁₋₆ alkyl), —S(O)(C₁₋₆ alkyl), —S(O)₂(C₁₋₆ alkyl), or —S(O)₂NH(C₁₋₆ alkyl);
n is an integer from 0-20;
p is 0, 1, 2, 3, 4, or 5; and
q is 0, 1, 2, 3, 4, or 5.

In some embodiments, each R₅, R₆, R₇, R₈, or R₉ is independently hydrogen, deuterium, halogen, —CN, —OR¹⁰, or C₁₋₁₀ alkyl. In some embodiments, each R₅, R₆, R₇, R₈, or R₉ is independently hydrogen, deuterium, halogen, methyl, ethyl, or isopropyl.

In some aspects, described herein is a compound of Formula II:

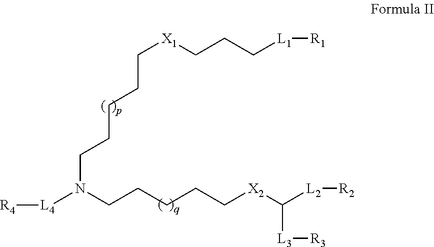

or its N-oxide, or a pharmaceutically acceptable salt thereof, wherein:
L₁ is substituted or unsubstituted linear —C₃₋₁₂ alkylene;
L₂ is substituted or unsubstituted linear —C₄₋₁₂ alkylene;
L₃ is substituted or unsubstituted linear —C₄₋₁₂ alkylene;
wherein if L₁, L₂, or L₃ is substituted, then L₁, L₂, or L₃ is substituted with 1-5 substituents selected from the group consisting of halogen, deuterium, —CN, linear or branched C₁₋₆ alkyl, C₁₋₂ haloalkyl, —C(═O)R¹⁰, —C(═O)N(R¹⁰)₂, —OR¹⁰, —N(R¹⁰)₂, substituted or unsubstituted C₃₋₆ cycloalkyl, and substituted or unsubstituted 3- to 6-membered heterocycloalkyl; wherein each substituted C₃₋₆ cycloalkyl and substituted 3- to 6-membered heterocycloalkyl is substituted with 1-5 R¹¹;
L₄ is substituted or unsubstituted —C₁₋₂₄ alkylene-; wherein L₄ is optionally substituted with 1 to 10 R¹¹;
X₁ is —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—;
X₂ is —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—;
each R₁, R₂, and R₃, is independently hydrogen, deuterium, halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl;
provided that at least one of R₁, R₂, and R₃ is not hydrogen or deuterium;
or when each R₁, R₂, and R₃ is hydrogen or deuterium, then at least one of L₁, L₂, L₃ is substituted with 1-5 substituents, and at least one of the substituents is not a linear C₁₋₁₀ alkyl substituted at a terminal carbon, or a deuterium;
R₄ is —OH, —OR¹⁰, —O—C(═O)—R¹⁰, —C(═O)—OR¹⁰, —O—C(═O)—OR¹⁰—, —C(═O)—O—C(═O)—R¹⁰, —O—C(═O)—C₀₋₉ alkylene-R¹¹, —(O—C₀₋₉ alkylene-)ₙR¹¹, —O—C(═O)—C₀₋₉ alkylene-N(R¹⁰)₂, —N(R¹⁰)₂, —CH(CH₃)—CH₂—OH, or —C(═O)—C₀₋₉ alkylene-N(R¹⁰)₂;
each R¹⁰ is independently hydrogen, C₁₋₁₀ alkyl, C₁₋₁₀ heteroalkyl, C₁₋₁₀ haloalkyl, C₃₋₁₀ heterocycloalkyl, C₂₋₆ alkenyl, C₂₋₆ alkynyl or C₃₋₁₀ cycloalkyl;
each R¹¹ is independently selected from the group consisting of hydrogen, deuterium, halogen, —CN, —C₁₋₆ alkyl, C₁₋₁₀ heteroalkyl, C₁₋₁₀ haloalkyl, C₃₋₁₀ heterocycloalkyl, C₂₋₆ alkenyl, C₂₋₆ alkynyl, —C(O)OH, —C(O)—O—C₁₋₆ alkyl, —C(O)—C₁₋₆ alkyl, —C(O)NH₂, —C(O)NH(C₁₋₆ alkyl), —C(O)N(C₁₋₆ alkyl)₂, —NH₂, —NH(C₁₋₆ alkyl), —N(C₁₋₆ alkyl)₂, —OH, —O—C₁₋₆ alkyl, —SH, —S(C₁₋₆ alkyl), —S(O)(C₁₋₆ alkyl), —S(O)₂(C₁₋₆ alkyl), or —S(O)₂NH(C₁₋₆ alkyl);
n is an integer from 0-20;
p is 0, 1, 2, 3, or 4; and
q is 0, 1, 2, 3, or 4.

In some embodiments, X₁ is —C(═O)—O—, —O—C(═O)—, —O—C(═O)—O—, —C(═O)—N(R¹⁰)—, or —N(R¹⁰)—C(═O)—. In another embodiments, X₂ is —C(═O)—O—, —O—C(═O)—, —O—C(═O)—O—, —C(═O)—N(R¹⁰)—, or —N(R¹⁰)—C(═O)—. In some embodiments, p is 1, 2, or 3; and q is 1, 2, or 3. In another embodiment, p is 1, and q is 1. In another embodiment, p is 1, and q is 3. In yet another embodiment, p is 3, and q is 1. In yet another embodiment, p is 3, and q is 3.

In some aspects, described herein is a compound of Formula III:

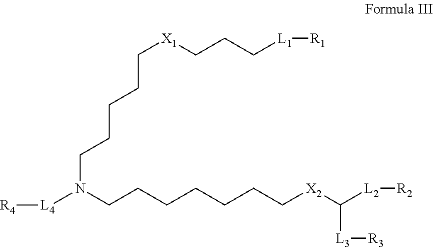

or its N-oxide, or a pharmaceutically acceptable salt thereof.

In another aspect, described herein is a compound of Formula III-a:

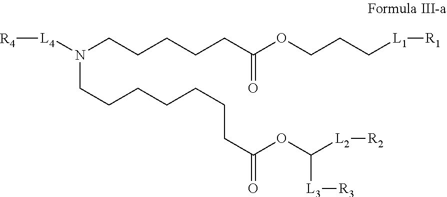

or its N-oxide, or a pharmaceutically acceptable salt thereof.

In some aspects, described herein is a compound of Formula IV:

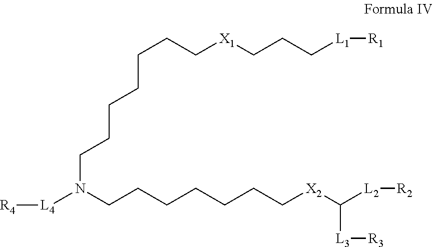

or its N-oxide, or a pharmaceutically acceptable salt thereof. In some embodiments, X₁ is —C(═O)—O— or —O—C(═O)—. In some embodiments, X₂ is —C(═O)—O— or —O—C(═O)—.

In another aspect, described herein is a compound of Formula IV-a:

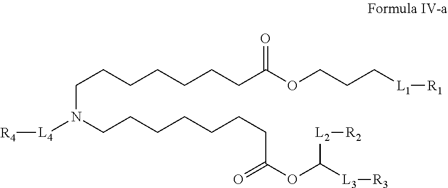

or its N-oxide, or a pharmaceutically acceptable salt thereof.

In some aspects, described herein is a compound of Formula V:

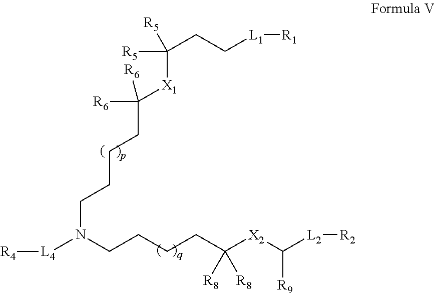

wherein:
L₁ is substituted or unsubstituted linear —C₃₋₁₂ alkylene or —C₃₋₁₂ alkenylalkylene;
L₂ is substituted or unsubstituted linear —C₄₋₁₂ alkylene or —C₃₋₁₂ alkenylalkylene;
wherein if L₁ or L₂ is substituted, then L₁ or L₂ is substituted with 1-5 substituents selected from the group consisting of halogen, deuterium, —CN, linear or branched C₁₋₁₀ alkyl, linear or branched C₁₋₁₀ heteroalkyl, C₁₋₂ haloalkyl, —C(═O)R¹⁰, —C(═O)N(R¹⁰)₂, —OR¹⁰, —N(R¹⁰)₂, substituted or unsubstituted C₃₋₆ cycloalkyl, and substituted or unsubstituted 3- to 10-membered heterocycloalkyl; wherein each substituted C₃₋₆ cycloalkyl and substituted 3- to 10-membered heterocycloalkyl is substituted with 1-5 R¹¹;
L₄ is substituted or unsubstituted —C₁₋₂₄ alkylene or —C₃₋₂₄ alkenylalkylene; wherein L₄ is optionally substituted with 1 to 10 R¹¹;
X₁ is —C(═O)—O—, —O—C(═O)—, —O—C(═O)—O—, —C(═O)—N(R¹⁹)—, —N(R¹⁰)—C(═O)—, S—S, —C(═O)—S—, or —C(C═S)—O—;
X₂ is —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—;
each R₁ and R₂ is independently hydrogen, deuterium, halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl;
R₄ is —OH, —OR¹⁰, —O—C(═O)—R¹⁰, —C(═O)—OR¹⁰, —O—C(═O)—OR¹⁰—, —C(═O)—O—C(═O)—R¹⁰, —O—C(═O)—C₀₋₉ alkylene-R¹¹, —(O—C₀₋₉ alkylene-)ₙR¹¹, —O—C(═O)—C₀₋₉ alkylene-N(R¹⁰)₂, —N(R¹⁰)₂, or —C(═O)—C₀₋₉ alkylene-N(R¹⁰)₂;
each R₅, R₆, R₈, or R₉ is independently hydrogen, deuterium, halogen, —CN, —OR¹⁰, —N(R¹⁰)₂, —O—C(═O)—R¹⁰, C₁₋₁₀ alkyl, C₁₋₁₀ heteroalkyl, C₃₋₁₀ cycloalkyl, C₃₋₁₀ heterocycloalkyl, C₂₋₆ alkenyl, or C₂₋₆ alkynyl;
each R¹⁰ is independently hydrogen, C₁₋₁₀ alkyl, C₁₋₁₀ heteroalkyl, C₁₋₁₀ haloalkyl, C₃₋₁₀ each R¹¹ is independently hydrogen, deuterium, halogen, —CN, C₁₋₁₀ alkyl, C₁₋₁₀ heteroalkyl, C₁₋₁₀ haloalkyl, C₃₋₁₀ cycloalkyl, C₃₋₁₀ heterocycloalkyl, C₂₋₆ alkenyl, C₂₋₆ alkynyl, —C(O)OH, —C(O)—O—C₁₋₆ alkyl, —C(O)—C₁₋₆ alkyl, —C(O)NH₂, —C(O)NH(C₁₋₆ alkyl), —C(O)N(C₁₋₆ alkyl)₂, —NH₂, —NH(C₁₋₆ alkyl), —N(C₁₋₆ alkyl)₂, —OH, —O—C₁₋₆ alkyl, —SH, —S(C₁₋₆ alkyl), —S(O)(C₁₋₆ alkyl), —S(O)₂(C₁₋₆ alkyl), or —S(O)₂NH(C₁₋₆ alkyl);
n is an integer from 0-20;
p is 0, 1, 2, 3, 4, or 5; and
q is 0, 1, 2, 3, 4, or 5.

In some embodiments, described herein is a compound of Formula VI, or an N-oxide, or a pharmaceutically acceptable salt thereof:

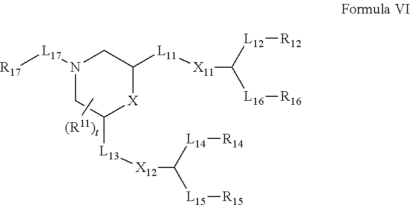

wherein:
X is O, S, or C(R¹¹)₂;
L₁₁ is substituted or unsubstituted linear —C₃₋₁₂ alkylene, wherein one or more methylene units of the group are optionally and independently replaced with —CR¹¹═CR¹¹—;
L₁₂ is substituted or unsubstituted linear —C₃₋₁₂ alkylene, wherein one or more methylene units of the group are optionally and independently replaced with —CR¹¹═CR¹¹—;
L₁₃ is substituted or unsubstituted linear —C₃₋₁₂ alkylene, wherein one or more methylene units of the group are optionally and independently replaced with —CR¹¹═CR¹¹;
L₁₄ is substituted or unsubstituted linear —C₃₋₁₂ alkylene, wherein one or more methylene units of the group are optionally and independently replaced with —CR¹¹═CR¹¹—;
L₁₅ is substituted or unsubstituted linear —C₀₋₁₂ alkylene, wherein one or more methylene units of the group are optionally and independently replaced with —CR¹¹═CR¹¹—;
L₁₆ is substituted or unsubstituted linear —C₀₋₁₂ alkylene, wherein one or more methylene units of the group are optionally and independently replaced with —CR¹¹═CR¹¹—;
wherein if L₁₁, L₁₂, L₁₃, L₁₄, L₁₅, or L₁₆ is substituted, then L₁, L₁₂, L₁₃, L₁₄, L₁₅, or L₁₆ is substituted with 1-5 substituents selected from the group consisting of halogen, deuterium, —CN, linear or branched C₁₋₆ alkyl, C₁₋₂ haloalkyl, —C(═O)R¹⁰, —C(═O)N(R¹⁰)₂, —OR¹⁰, —N(R¹⁰)₂, substituted or unsubstituted C₃₋₆ cycloalkyl, and substituted or unsubstituted 3- to 6-membered heterocycloalkyl; wherein each substituted C₃₋₆ cycloalkyl and substituted 3- to 6-membered heterocycloalkyl is substituted with 1-5 R¹¹;
X₁₁ is a covalent bond, —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—;
X₁₂ is a covalent bond, —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—;
each R₁₂, R₁₄, R₁₅ and R₁₆, is independently hydrogen, deuterium, halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl;
L₁₇ is substituted or unsubstituted —C₁₋₂₄ alkylene-; wherein L₁₇ is optionally substituted with 1 to 10 R¹¹;
R₁₇ is —OH, —OR¹⁰, —O—C(═O)—R¹⁰, —C(═O)—OR¹⁰, —O—C(═O)—OR¹⁰—, —C(═O)—O—C(═O)—R¹⁰, —O—C(═O)—C₀₋₉ alkylene-R¹¹, —(O—C₀₋₉ alkylene-)ₙR¹¹, —O—C(═O)—C₀₋₉ alkylene-N(R¹⁰)₂, —N(R¹⁰)₂, —CH(CH₃)—CH₂—OH, or —C(═O)—C₀₋₉ alkylene-N(R¹⁰)₂;
each R¹⁰ is independently hydrogen, C₁₋₁₀ alkyl, C₁₋₁₀ heteroalkyl, C₁₋₁₀ haloalkyl, C₃₋₁₀ heterocycloalkyl, C₂₋₆ alkenyl, C₂₋₆ alkynyl or C₃₋₁₀ cycloalkyl;
each R¹¹ is independently selected from the group consisting of hydrogen, deuterium, halogen, —CN, —C₁₋₆ alkyl, C₁₋₁₀ heteroalkyl, C₁₋₁₀ haloalkyl, C₃₋₁₀ heterocycloalkyl, C₂₋₆ alkenyl, C₂₋₆ alkynyl, —C(O)OH, —C(O)—O—C₁₋₆ alkyl, —C(O)—C₁₋₆ alkyl, —C(O)NH₂, —C(O)NH(C₁₋₆ alkyl), —C(O)N(C₁₋₆ alkyl)₂, —NH₂, —NH(C₁₋₆ alkyl), —N(C₁₋₆ alkyl)₂, —OH, —O—C₁₋₆ alkyl, —SH, —S(C₁₋₆ alkyl), —S(O)(C₁₋₆ alkyl), —S(O)₂(C₁₋₆ alkyl), or —S(O)₂NH(C₁₋₆ alkyl); and tis 0, 1, 2, 3, or 4.

In some embodiments, each variable group of Formula V is independently as described herein.

In some embodiments, at least one of R₁, R₂, and R₃ is not hydrogen or deuterium. In some embodiments, R₁, R₂, and R₃ are each independently halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl. In some embodiments, at least one of R₁, R₂, and R₃ is halogen, C₂₋₆ alkenyl, or branched C₃₋₁₀ alkyl. In some embodiments, at least one of R₁, R₂, and R₃ is fluoro or isopropyl. In some aspects, R₁ is halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl. In one aspect, R₁ is halogen, —CN, —OR¹⁰, —N(R¹⁰)₂, C₂₋₆ alkenyl, or branched C₃₋₁₀ alkyl. In another aspect, R₁ is halogen, —OR¹⁰, C₂₋₆ alkenyl, or branched C₃₋₁₀ alkyl. In yet another aspect, R₁ is halogen, —OH, isopropyl, isobutyl, isopentyl, sec-butyl, tert-butyl, tert-pentyl, or tert-hexyl. In some embodiments, R₁ is halogen or isopropyl, isobutyl, sec-butyl, or tert-butyl. In some embodiments, R₁ is halogen or isopropyl. In some embodiments, R₁ is F. In some embodiments, R₁ is Cl. In some embodiments, R₁ is Br. In some embodiments, R₁ is I. In some embodiments, R₁ is isopropyl. In some embodiments, R₁ is C₂₋₆ alkenyl. In some embodiments, R₁ is

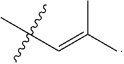 [SMILES: *C=C(C)C]

In some aspects, R₂ is halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl. In one aspect, R₂ is halogen, —CN, —OR¹⁰, —N(R¹⁰)₂, C₂₋₆ alkenyl, or branched C₃₋₁₀ alkyl. In another aspect, R₂ is halogen, —OR¹⁰, C₂₋₆ alkenyl, or branched C₃₋₁₀ alkyl. In yet another aspect, R₂ is halogen, —OH, isopropyl, isobutyl, isopentyl, sec-butyl, tert-butyl, tert-pentyl, or tert-hexyl. In some embodiments, R₂ is halogen or isopropyl, isobutyl, sec-butyl, or tert-butyl. In some embodiments, R₂ is halogen or isopropyl. In some embodiments, R₂ is F. In some embodiments, R₂ is Cl. In some embodiments, R₂ is Br. In some embodiments, R₂ is I. In some embodiments, R₂ is isopropyl. In some aspects, R₃ is halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl. In one aspect, R₃ is halogen, —CN, —OR¹⁰, —N(R¹⁰)₂, C₂₋₆ alkenyl, or branched C₃₋₁₀ alkyl. In another aspect, R₃ is halogen, —OR¹⁰, C₂₋₆ alkenyl, or branched C₃₋₁₀ alkyl. In another aspect, R₃ is halogen, —OH, isopropyl, isobutyl, isopentyl, sec-butyl, tert-butyl, tert-pentyl, or tert-hexyl. In some embodiments, R₃ is halogen or isopropyl, isobutyl, sec-butyl, or tert-butyl. In some embodiments, R₃ is halogen or isopropyl. In some embodiments, R₃ is F. In some embodiments, R₃ is Cl. In some embodiments, R₃ is Br. In some embodiments, R₃ is I. In some embodiments, R₃ is isopropyl. In some embodiments, at least one of R₂ and R₃ is halogen, —CN, —OR¹⁰, —N(R¹⁰)₂, C₂₋₆ alkenyl, or branched C₃₋₁₀ alkyl. In some aspects, R₃ is halogen, —OR¹⁰, or branched C₃₋₁₀ alkyl; and R₁ and R₂ are hydrogen. In another aspect, R₃ is halogen, —OR¹⁰, or branched C₃₋₁₀ alkyl; R₂ is halogen, —OR¹⁰, or branched C₃₋₁₀ alkyl and R₁ is hydrogen. In another aspect, R₃ is halogen, —OR¹⁰, or branched C₃₋₁₀ alkyl; R₂ is halogen, —OR¹⁰, or branched C₃₋₁₀ alkyl and R₁ is C₂₋₆ alkenyl. In some aspects, R₃ is F or branched C₃₋₁₀ alkyl; and R₁ and R₂ are hydrogen. In another aspect, R₃ is F or branched C₃₋₁₀ alkyl; R₂ is F or branched C₃₋₁₀ alkyl and R₁ is hydrogen. In another aspect, R₃ is F or branched C₃₋₁₀ alkyl; R₂ is F or branched C₃₋₁₀ alkyl and R₁ is

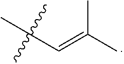 [SMILES: *C=C(C)C]

In some aspects, R₃ is Cl or branched C₃₋₁₀ alkyl; and R₁ and R₂ are hydrogen. In another aspect, R₃ is Cl or branched C₃₋₁₀ alkyl; R₂ is Cl or branched C₃₋₁₀ alkyl and R₁ is hydrogen. In another aspect, R₃ is Cl or branched C₃₋₁₀ alkyl; R₂ is F or branched C₃₋₁₀ alkyl and R₁ is

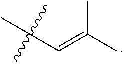 [SMILES: *C=C(C)C]

In some embodiments, R₃ is halogen, —OR¹⁰, C₂₋₆ alkenyl, or branched C₃₋₁₀ alkyl; and R₁ and R₂ are hydrogen. In some embodiments, R₃ is halogen, —OR¹⁰, C₂₋₆ alkenyl, or branched C₃₋₁₀ alkyl; R₂ is halogen, —OR¹⁰, C₂₋₆ alkenyl, or branched C₃₋₁₀ alkyl and R₁ is hydrogen. In some embodiments, R₁ is H; and R₂ and R₃ are each independently halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl. In some embodiments. R₁ is H; and R₂ and R₃ are each independently halogen, —OR¹⁰, C₂₋₆ alkenyl, or branched C₃₋₆ alkyl. In some embodiments, R₁ is H; R₂ is H, halogen, isopropyl, isobutyl, sec-butyl, or tert-butyl; and R₃ is halogen, —OR¹⁰, or isopropyl, isobutyl, sec-butyl, or tert-butyl. In some embodiments, R₁ is halogen, —OR¹⁰, C₂₋₆ alkenyl, or branched C₃₋₆ alkyl; and R₂ and R₃ are each independently H.

In some embodiments, each R₁, R₂, and R₃ is hydrogen or deuterium, then at least one of L₁, L₂, L₃ is substituted with 1-5 substituents, and at least one of the substituents is not a linear C₁₋₁₀ alkyl substituted at a terminal carbon of L₁, L₂, L₃, or a deuterium. In some aspects, L₁ is substituted or unsubstituted —C₄₋₁₀ alkylene, or —C₃₋₁₂ alkenylalkylene. In another aspect, L₁ is —C₄₋₁₀ alkylene or —C₃₋₁₂ alkenylalkylene, substituted with halogen, linear or branched C₁₋₆ alkyl, or C₁₋₂ haloalkyl. In some embodiments, L₁ is —C₄₋₁₀ alkylene or —C₃₋₁₂ alkenylalkylene, substituted with fluoro, methyl, or isopropyl. In some embodiments, each L₂ and L₃ is independently substituted or unsubstituted —C₄₋₁₀ alkylene, or —C₃₋₁₂ alkenylalkylene. In one aspect, wherein L₂ and L₃ are the same. In another aspect, L₂ and L₃ are different. In some embodiments, each L₂ and L₃ is independently substituted —C₄₋₁₀ alkylene, or —C₃₋₁₂ alkenylalkylene, wherein the substituent is halogen, or linear or branched C₁₋₆ alkyl. In some embodiments, each L₂ and L₃ is independently a substituted —C₄₋₁₀ alkylene, or —C₃₋₁₂ alkenylalkylene, wherein —C₄₋₁₀ alkylene is substituted with fluoro, methyl, or isopropyl.

In some aspects, L₄ is substituted or unsubstituted —C₁₋₁₂ alkylene, or —C₃₋₂₄ alkenylalkylene; wherein L₄ is optionally substituted with 1 to 10 R¹¹. In some embodiments, L₄ is substituted or unsubstituted —C₁₋₆ alkylene, or —C₃₋₁₂ alkenylalkylene; wherein L₄ is optionally substituted with 1 to 10 R¹¹. In another embodiment, L₄ is —CH₂—, —CH₂—CH₂—, —CH₂—CH₂—CH₂—, —CH₂—(CH₂)₂—CH₂—, —CH₂—(CH₂)₃—CH₂—, or —CH₂—(CH₂)₄—CH₂—. In another embodiment, L₄ is —CH₂—, —CH₂—CH₂—, —CH₂—CH₂—CH₂—, —CH₂—CH(CH₃)—CH₂—, —CH₂—(CH₂)₂—CH₂—, —CH₂—(CH₂)₃—CH₂—, or —CH₂—(CH₂)₄—CH₂—. In some embodiments, L₄ is —CH₂—, —CH₂—CH₂—, or —CH₂—CH₂—CH₂—. In some embodiments, L₄ is —CH₂—CH(CH₃)—CH₂—. In some embodiments, L₄ is —CH₂—CH(CH₃)—CH₂—. In some embodiments, L₄ is —CD₂—CD₂—. In some embodiments, R₄ is —OH, —OR¹⁰, —O—C(═O)—R¹⁰, —C(═O)—OR¹⁰, —O—C(═O)—C₀₋₉ alkylene-N(R¹¹)₂, —O—C(═O)—C₀₋₉ alkylene-R¹¹, —(O—C₀₋₉ alkylene-)ₙR¹¹, —CH(CH₃)—CH₂—OH, or —C(═O)—C₀₋₉ alkylene-N(R¹¹)₂. In one aspect, R₄ is —OH, —CH(CH₃)—CH₂—OH, or —O—C(═O)—(CH₂)₃—N(Me)₂. In some embodiments, R₄ is —OH. In some embodiments, R₄ is —N(R¹¹)₂. In some embodiments, R₄ is —N(R¹¹)₂ wherein each R¹¹ is independently hydrogen or C₁₋₆ alkyl. In some embodiments, R₄ is —NMe₂.

Described herein, some aspects, is a compound having the structure:

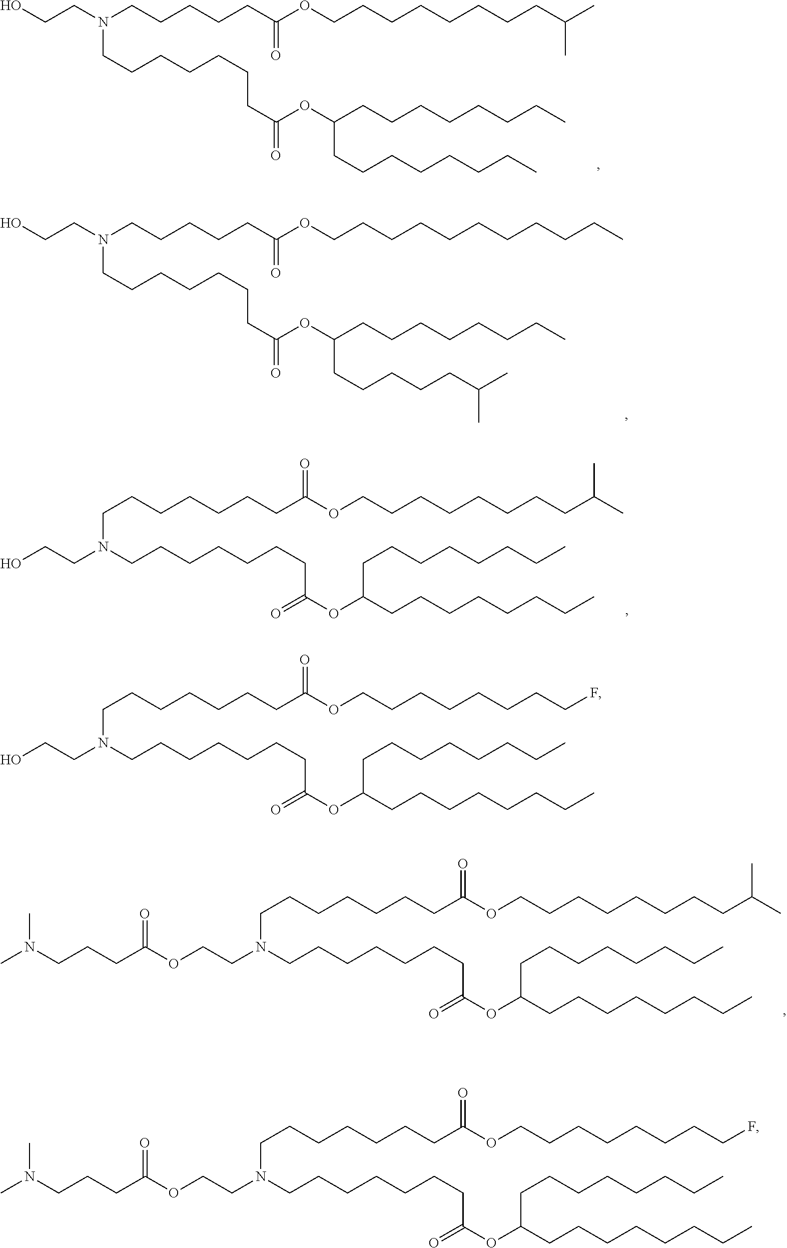

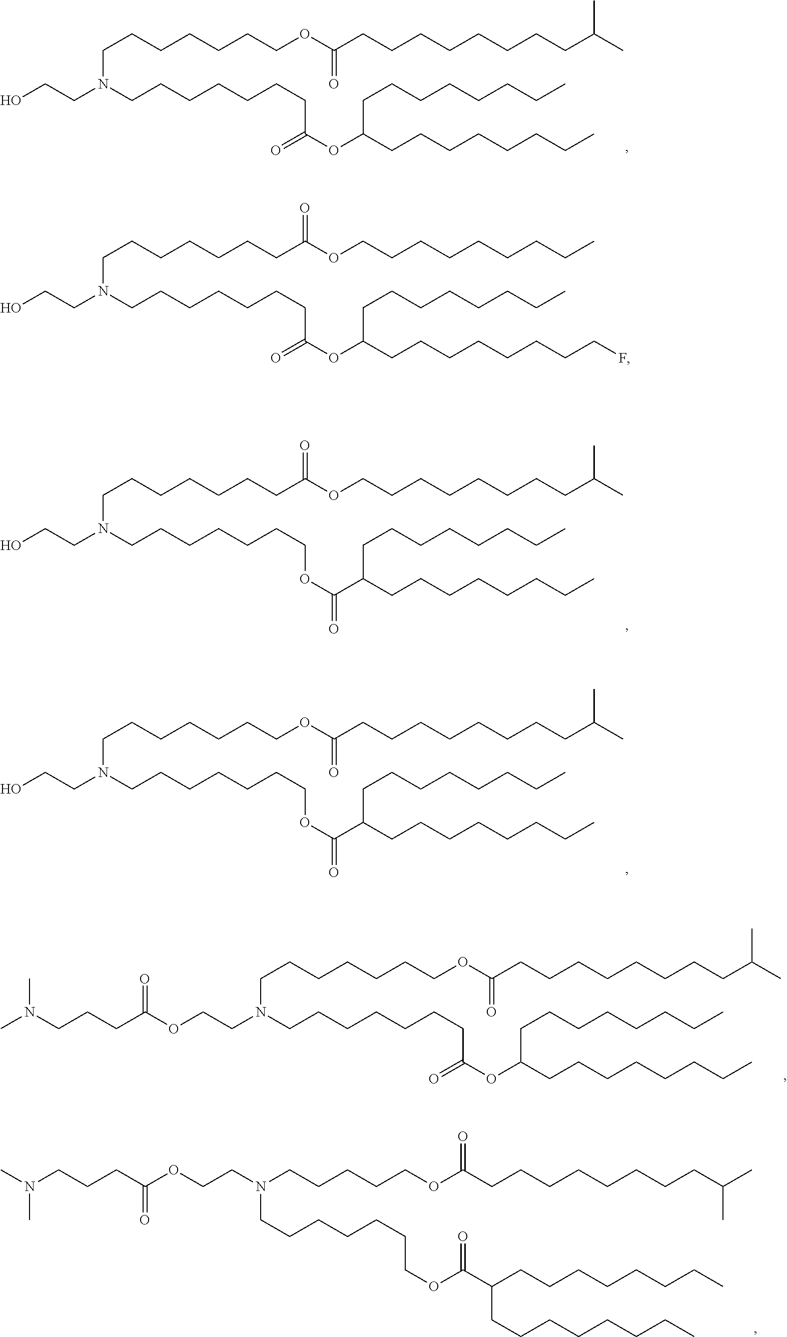

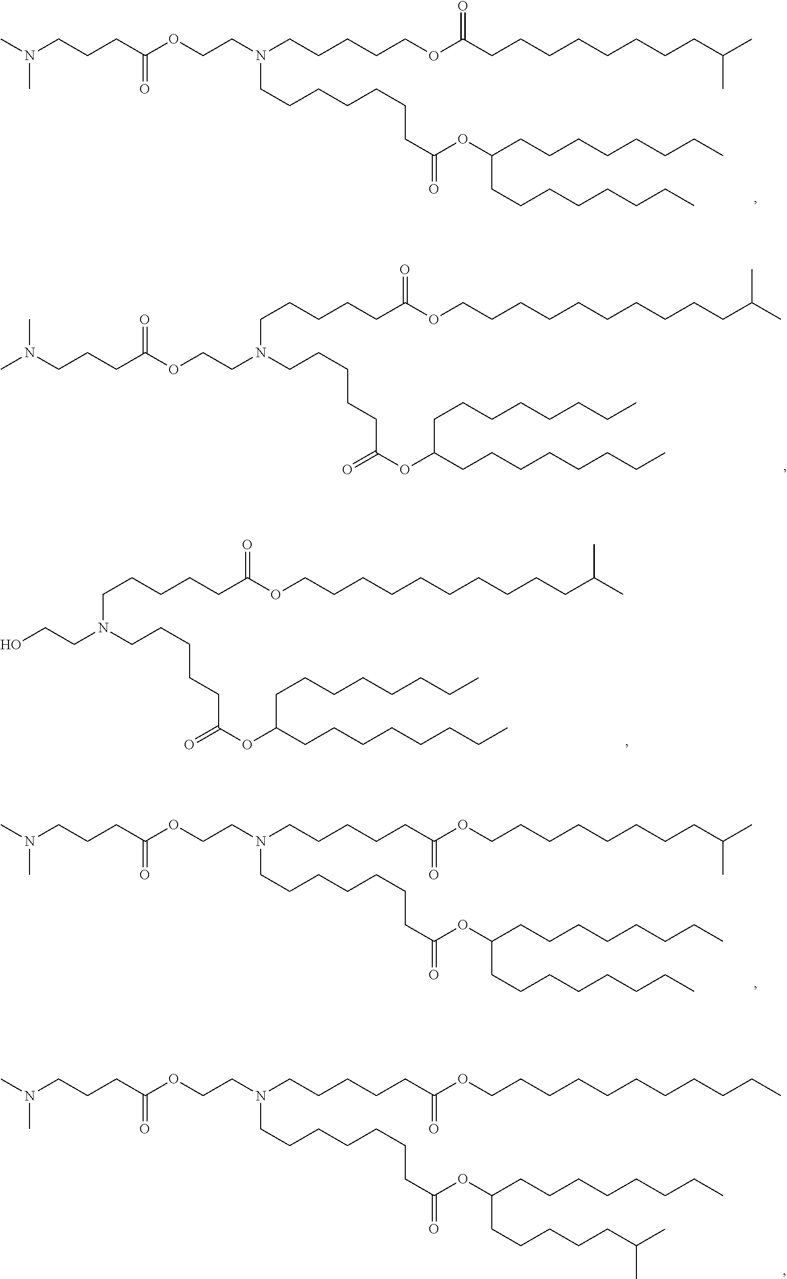

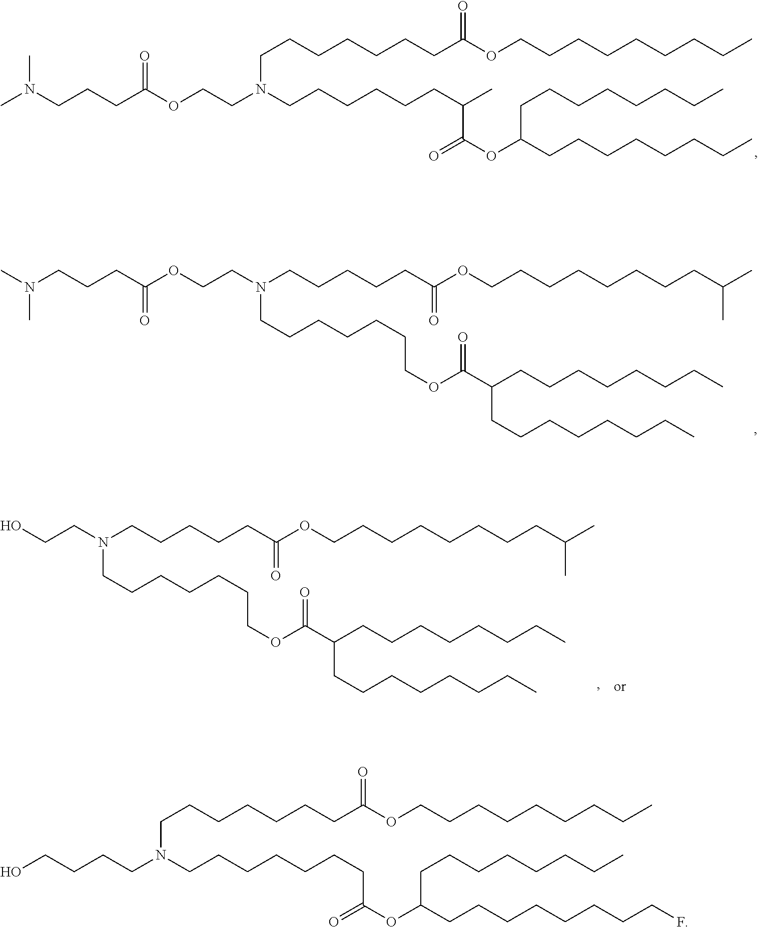

Described herein, in some aspects, is a compound having the structure:

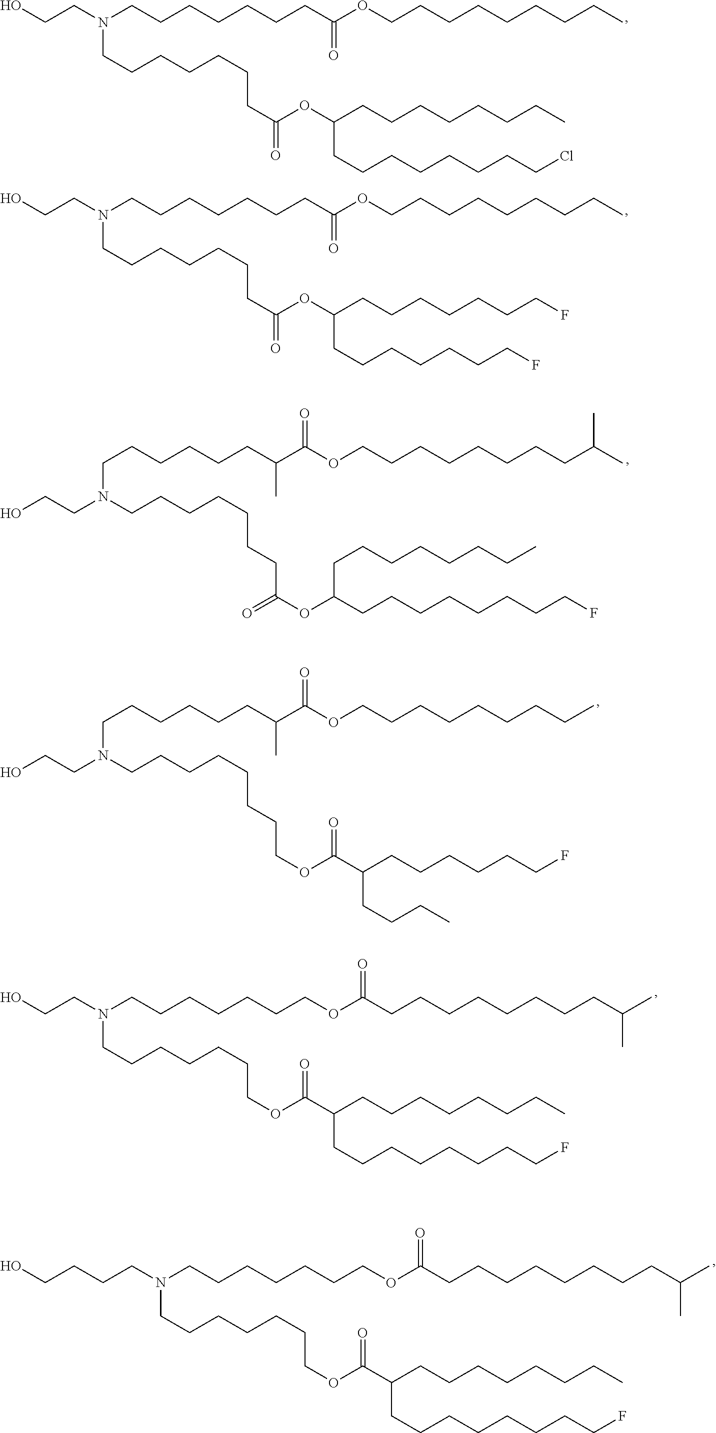

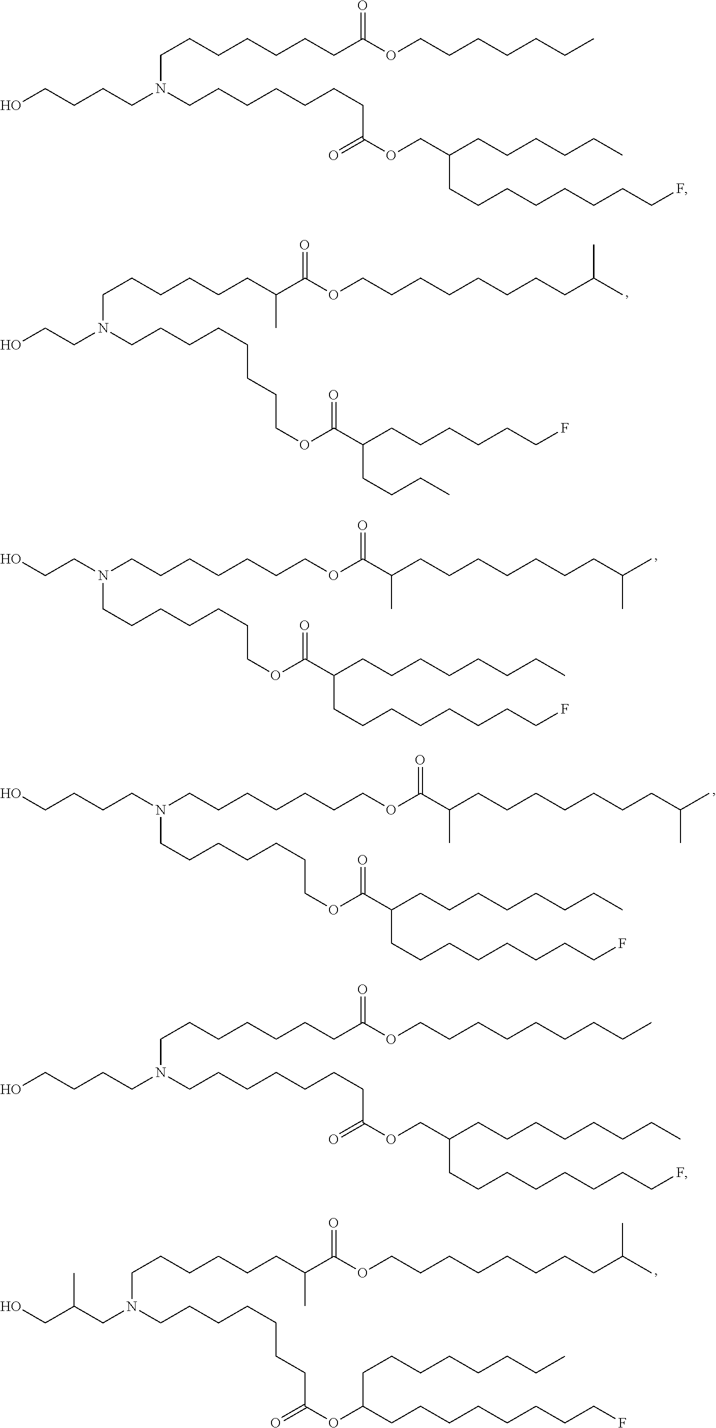

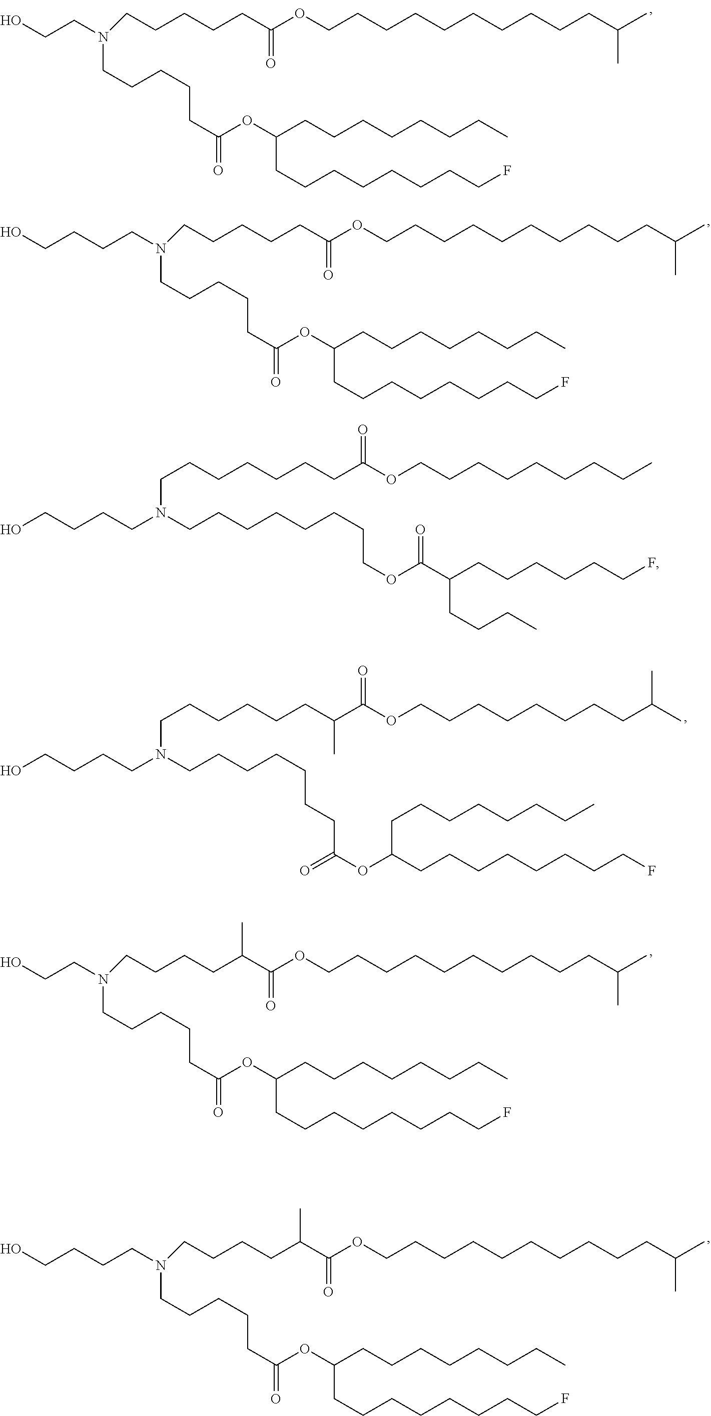

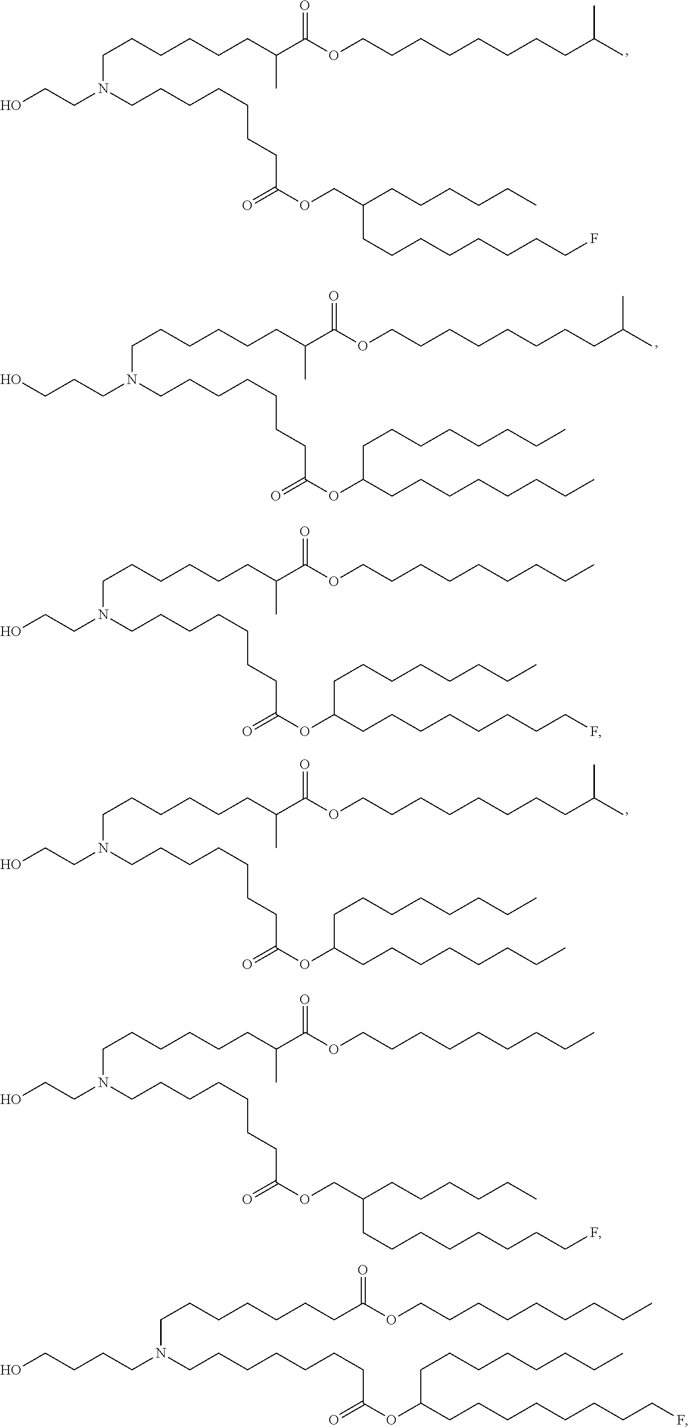

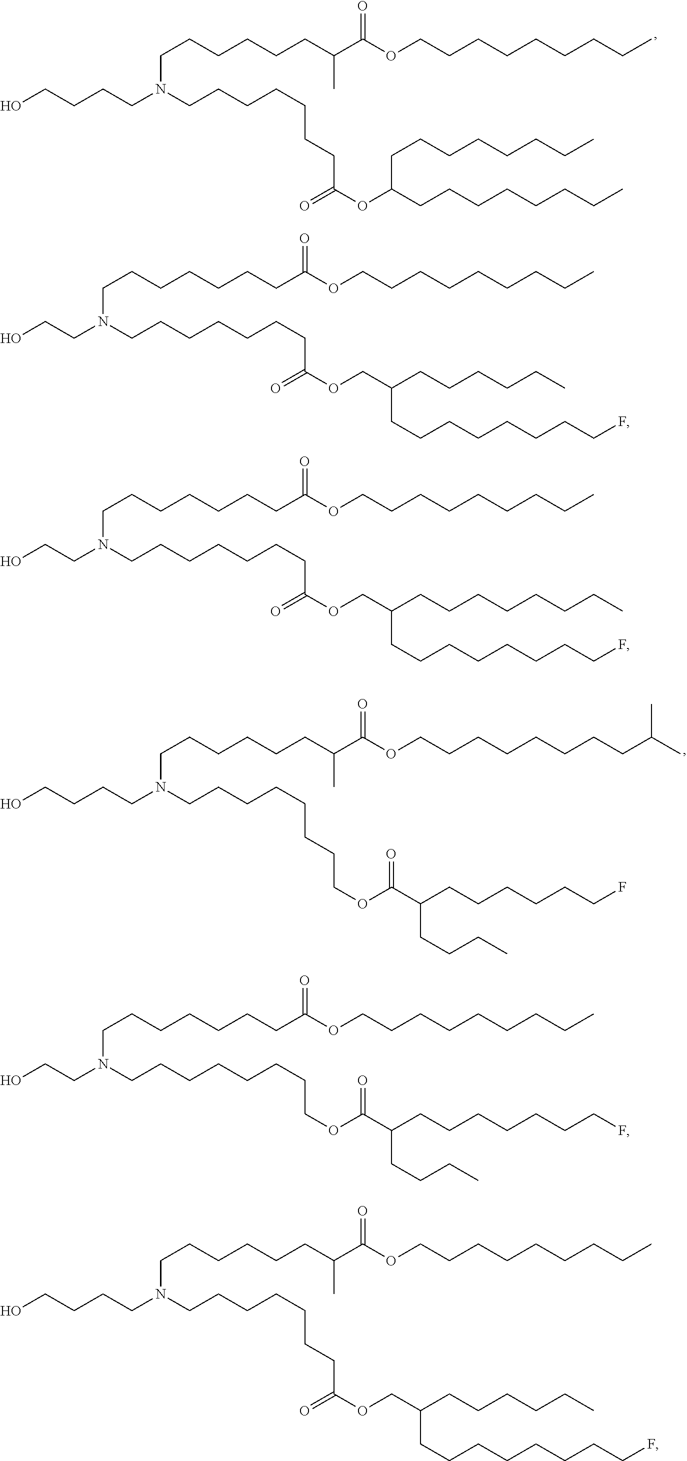

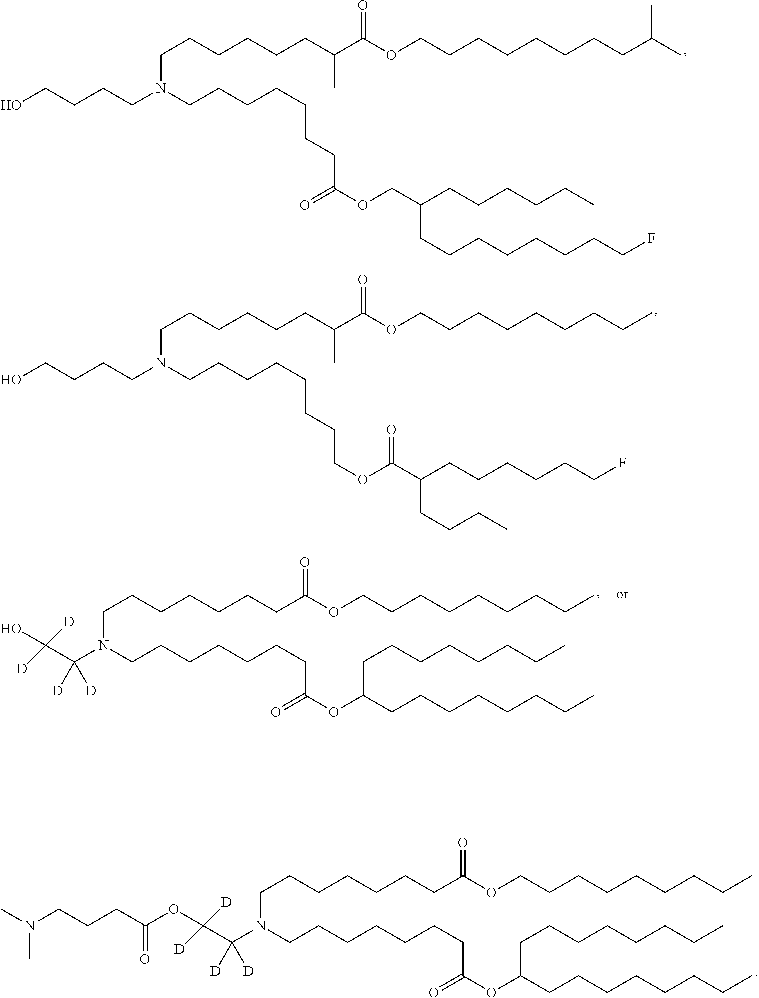

Described herein, in some aspects, is a nanoparticle composition comprising a lipid component, wherein the lipid component comprises a compound disclosed herein (e.g., a compound of Formula I′, I′-a, I-a, I-b, II, III, III-a, IV, IV-a, V, or VI).

Described herein, in some aspects, is a lipid component further comprises a structural lipid. In some aspects, the structural lipid comprises cholesterol, fecosterol, sitosterol, ergosterol, campesterol, stigmasterol, brassicasterol, tomatidine, ursolic acid, or alpha-tocopherol. In some embodiments, the lipid component further comprises a PEG lipid. In some aspect, the PEG lipid is a PEG-modified phosphatidy-lethanolamine, a PEG-modified phosphatidic acid, a PEG-modified ceramide, a PEG-modified dialkylamine, a PEG-modified diacylglycerol, or a PEG-modified dialkylglycerol. In some embodiments, the lipid component further comprises a cationic and/or ionizable lipid. In some embodiments, the cationic and/or ionizable lipid is 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), or (2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)). In some aspects, the lipid component further comprises a phospholipid, a structural lipid, and a PEG lipid. In some embodiments, the lipid component comprises about 30% to about 60% of the compounds disclosed herein (e.g., a compound of Formula I′, I′-a, I-a, I-b, II, III, III-a, IV, IV-a, V, or VI), about 0% to about 30% phospholipid, about 20% to about 50% structural lipid, and about 0% to about 10% PEG lipid. In some embodiments, the nanoparticle composition of compounds of Formula I′, I′-a, I-a, I-b, II, III, III-a, IV, IV-a, V, or VI, further comprising a therapeutic and/or prophylactic agent. In some aspects, the therapeutic and/or prophylactic agent is nucleic acid. In some embodiments, therapeutic and/or prophylactic agent is ribonucleic acid (RNA). In some embodiments, RNA is a small interfering RNA (siRNA), an asymmetrical interfering RNA (aiRNA), a microRNA (miRNA), a Dicer-substrate RNA (dsRNA), a small hairpin RNA (shRNA), or a messenger RNA (mRNA). In another embodiment, the RNA is mRNA. In some embodiments, the mRNA comprises at least one nucleic acid modification. In some embodiments, at least one nucleic acid modification comprises a N1-methylpseudouridine (m1ψ).

Described herein, in some aspects, provided herein is a pharmaceutical composition comprising the nanoparticle composition of compounds of Formula I′, I′-a, I-a, I-b, II, III, III-a, IV, IV-a, V, or VI, and a pharmaceutically acceptable carrier.

Described herein, in some aspects, is a method of delivering a therapeutic agent to a subject in need thereof, the method comprising administering to the subject a nanoparticle composition disclosed herein, or a pharmaceutical composition, thereby the therapeutic agent is delivered to the subject. In some embodiments, disclosed herein is a method of producing a polypeptide of interest in a cell, the method comprising contacting the cell with a nanoparticle composition of compounds of Formula I′, I′-a, I-a, I-b, II, III, III-a, IV, IV-a, V, or VI, or a pharmaceutical composition, wherein the therapeutic agent is an mRNA, wherein the mRNA encodes the polypeptide of interest. In some embodiments, the mRNA is capable of being translated in the cell, thereby producing the polypeptide of interest.

In some aspects, disclosed herein is a method of selectively delivering a therapeutic agent to a mammalian organ, the method comprising administering to a mammal a nanoparticle composition of compounds of Formula I′, I′-a, I, I-a, I-b, II, III, III-a, IV, IV-a, V, or VI, or a pharmaceutical composition. In some embodiments, administering the nanoparticle composition or pharmaceutical composition comprises contacting the mammalian organ with the nanoparticle composition, thereby delivering the therapeutic agent to the organ. In some aspects, the therapeutic agent is delivered to the organ.

In some embodiments, disclosed herein is method of treating a disease or disorder in a subject in need thereof, the method comprising administering to the subject a therapeutically effective amount of a nanoparticle composition of compounds of Formula I′, I′-a, I, I-a, I-b, II, III, III-a, IV, IV-a, V, or VI, or a pharmaceutical composition of claim of the nanoparticle composition. In some embodiments, the disease or disorder is cancer.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates transfection of AML12 Cell Line with FLuc mRNA LNPs. Cells were transfected with 200 ng of Fluc mRNA encapsulated in LNPs and expression analyzed 24 hours later. Transfections were performed in triplicates.

FIG. 2 illustrates Cell Viability of AML12 Cell Line during transfection of FLuc mRNA LNPs. Cells were transfected with 200 ng of Fluc mRNA encapsulated in LNPs and viability assessed with Promega's CellTiter-Glo® Luminescent Cell Viability Assay. Transfections were performed in triplicates.

FIG. 3 illustrates results of transfection of B16F10 Cell Line with FLuc mRNA LNPs. Cells were transfected with 200 ng of Fluc mRNA encapsulated in LNPs and expression analyzed 24 hours later. Transfections were performed in triplicates.

FIG. 4 illustrates results of Cell Viability of B16F10 Cell Line during transfection of FLuc mRNA LNPs. Cells were transfected with 200 ng of Fluc mRNA encapsulated in LNPs and viability assessed with Promega's CellTiter-Glo® Luminescent Cell Viability Assay. Transfections were performed in triplicates.

FIG. 5 illustrates results of transfection of MC38.K Cell Line with FLuc mRNA LNPs. Cells were transfected with 200 ng of Fluc mRNA encapsulated in LNPs and expression analyzed 24 hours later. Transfections were performed in triplicates.

FIG. 6 illustrates results of Cell Viability of MC38.K Cell Line during transfection of FLuc mRNA LNPs. Cells were transfected with 200 ng of Fluc mRNA encapsulated in LNPs and viability assessed with Promega's CellTiter-Glo® Luminescent Cell Viability Assay. Transfections were performed in triplicates.

FIG. 7 illustrates results of Ex-Vivo Luminescence Signals From Liver. 20-gram CD1 Mice (Naïve) were injected with Fluc mRNA LNPs through tail-vein injection (1.0 mg/kg) on Day 0 and Day 2. Takedown and IVIS imaging was performed 18 hours post second dose (Day 3). n=5 mice per group.

FIG. 8 illustrates analysis of Fluc Expression as a function of lipid nanoparticle surface pKa. Arrow denotes benchmark expression per luminescent intensity.

FIG. 9 illustrates results of bodyweight comparison between two fluoro-functionalized ionizable lipids. Varying the position of fluoro moieties give significant contrasts in bodyweights and tolerance.

FIG. 10 illustrates results of transfection of AML12, B16F10, and MC38.K Cell Lines with FLuc mRNA LNPs. Cells were transfected with 200 ng of Fluc mRNA encapsulated in LNPs and expression analyzed 24 hours later. Transfections were performed in triplicates.

FIG. 11 illustrates results of Cell Viability of AML12, B16F10, and MC38.K Cell Lines during transfection of FLuc mRNA LNPs. Cells were transfected with 200 ng of Fluc mRNA encapsulated in LNPs and viability assessed with Promega's CellTiter-Glo® Luminescent Cell Viability Assay. Transfections were performed in triplicates.

FIG. 12 illustrates results of Absolute Reticulocyte, Red Blood Cell, and Hematocrit Counts along with hEPO Protein Expression (6 hours) after one 0.3 mg/kg IV-infusion of hEPO mRNA LNPs in Macaca fascicularis Cynomolgus monkeys. N=3.

FIG. 13 illustrates results of body weight, temperature, and liver blood chemistry of Macaca fascicularis Cynomolgus monkeys after one 0.3 mg/kg IV-infusion of hEPO mRNA LNPs. N=3.

FIG. 14 illustrates results of body weight, temperature, and liver blood chemistry of Macaca fascicularis Cynomolgus monkeys over the course of four weeks after weekly infusion of 0.1 mg/kg hEPO mRNA LNPs. N=3.

FIG. 15 illustrates results of hematocrit (HCT) and Absolute Reticulocyte Counts of Macaca fascicularis Cynomolgus monkeys over the course of four weeks after weekly infusion of 0.1 mg/kg hEPO mRNA LNPs. Treatments were on Days 0, 7, 14, and 21 and administered intravenously. N=3.

FIG. 16 illustrates results of quantification of serum hEPO after weekly treatments of 0.1 mg/kg of hEPO mRNA-LNPs in Macaca fascicularis Cynomolgus monkeys N=3.

FIG. 17 illustrates results of quantification of Compound 23 in Macaca fascicularis Cynomolgus monkey serum after weekly treatments of 0.1 mg/kg of hEPO-mRNA LNPs. N=3.

FIG. 18 illustrates results of comparison study on the tolerability of Compound 23 and Benchmark 1.

FIG. 19 illustrates results of an comparison study on the intratumor delivery efficiency of Compound 23 and Benchmark 1.

FIG. 20 illustrates results of an in vitro comparison study on Firefly Luciferase expression of LNPs comprising various pairs of fluoro v. non-fluoro compounds.

FIG. 21 illustrates transfection of AML12 Cell Line with FLuc mRNA LNPs. Cells were transfected with 200 ng of Fluc mRNA encapsulated in LNPs and expression analyzed 24 hours later. Transfections were performed in triplicates.

FIG. 22 illustrates Cell Viability of AML12 Cell Line during transfection of FLuc mRNA LNPs. Cells were transfected with 200 ng of Fluc mRNA encapsulated in LNPs and viability assessed with Promega's CellTiter-Glo® Luminescent Cell Viability Assay. Transfections were performed in triplicates.

FIG. 23 illustrates results of transfection of B16F10 Cell Line with FLuc mRNA LNPs. Cells were transfected with 200 ng of Fluc mRNA encapsulated in LNPs and expression analyzed 24 hours later. Transfections were performed in triplicates.

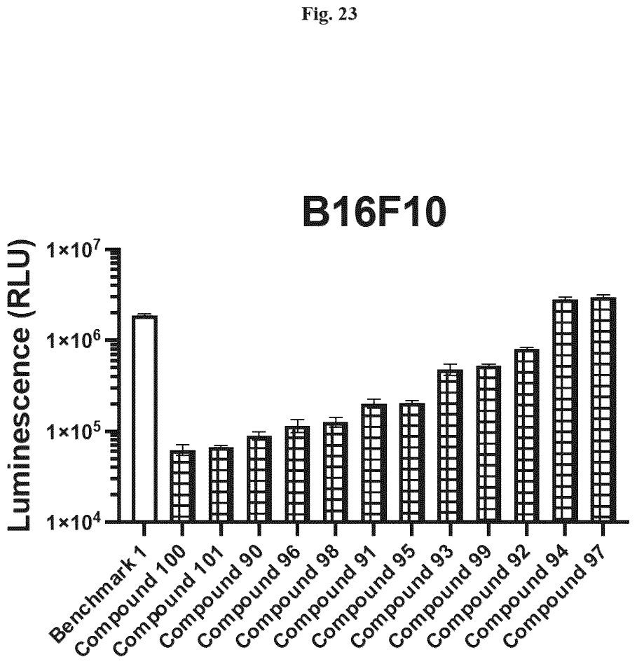

FIG. 24 illustrates results of Cell Viability of B16F10 Cell Line during transfection of FLuc mRNA LNPs. Cells were transfected with 200 ng of Fluc mRNA encapsulated in LNPs and viability assessed with Promega's CellTiter-Glo® Luminescent Cell Viability Assay Transfections were performed in triplicates.

FIG. 25 illustrates results of transfection of MC38.K Cell Line with FLuc mRNA LNPs. Cells were transfected with 200 ng of Fluc mRNA encapsulated in LNPs and expression analyzed 24 hours later. Transfections were performed in triplicates.

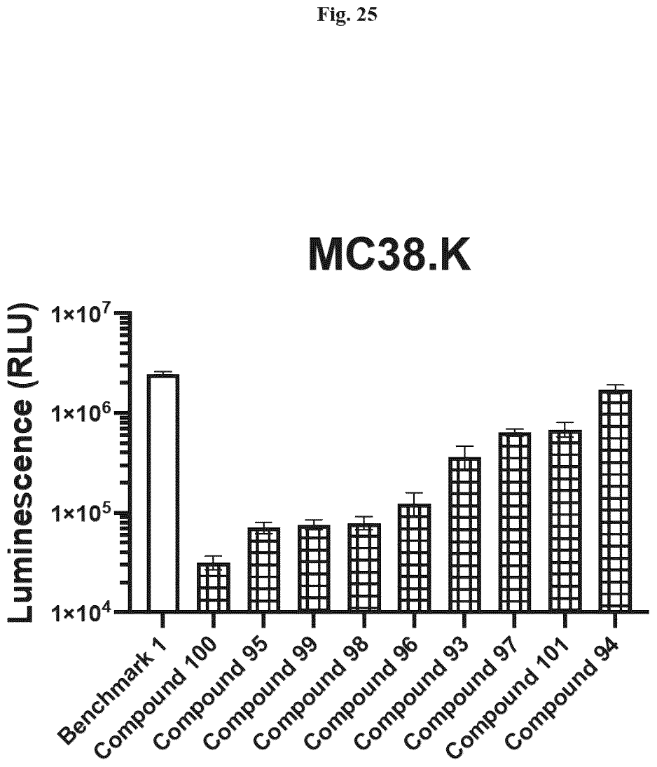

FIG. 26 illustrates results of Cell Viability of MC38.K Cell Line during transfection of FLuc mRNA LNPs. Cells were transfected with 200 ng of Fluc mRNA encapsulated in LNPs and viability assessed with Promega's CellTiter-Glo® Luminescent Cell Viability Assay. Transfections were performed in triplicates.

FIG. 27 illustrates results of Ex-Vivo Luminescence Signals From Liver. 20-gram CD1 Mice (Naïve) were injected with Fluc mRNA LNPs through tail-vein injection (1.0 mg/kg) on Day 0 and Day 2. Takedown and IVIS imaging was performed 18 hours post second dose (Day 3). n=5 mice per group.

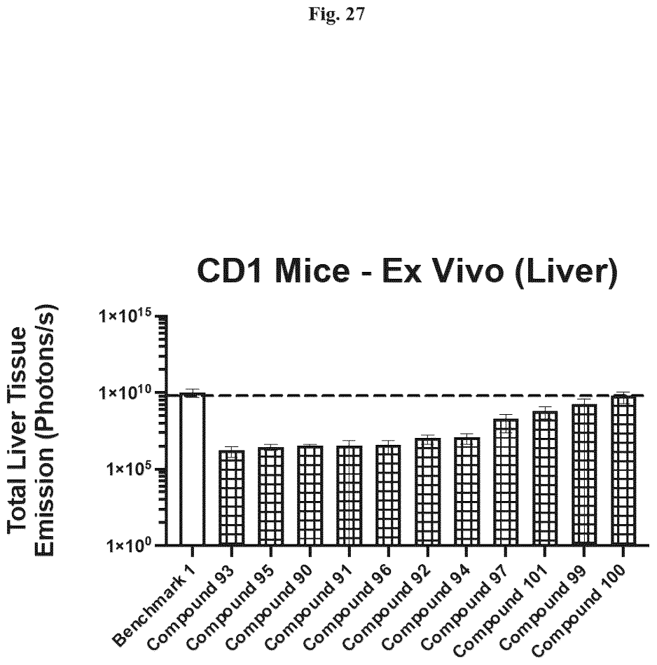

FIG. 28 illustrates results of Ex-Vivo Luminescence Signals From Spleen. 20-gram CD1 Mice (Naïve) were injected with Fluc mRNA LNPs through tail-vein injection (1.0 mg/kg) on Day 0 and Day 2. Takedown and IVIS imaging was performed 18 hours post second dose (Day 3). n=5 mice per group.

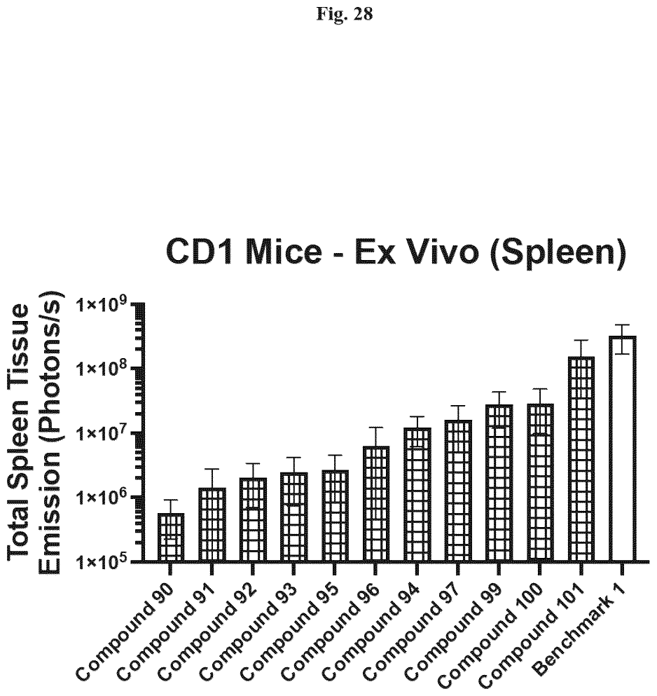

FIG. 29 illustrates analysis of Fluc Expression as a function of lipid nanoparticle surface pKa. Arrow denotes benchmark expression per luminescent intensity.

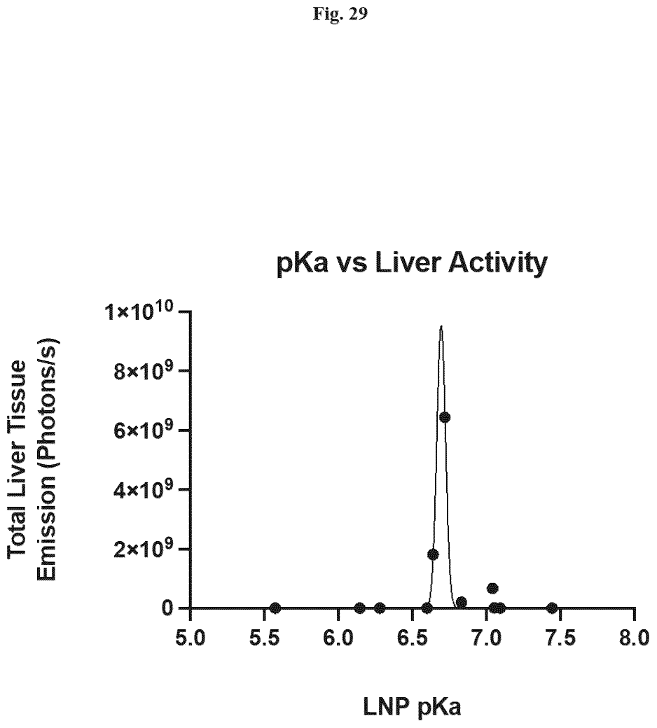

FIG. 30 illustrates bodyweight, white blood cell, and lymphocyte over the course of three weeks in a tolerability study.

FIG. 31 illustrates Ex-Vivo luminescence of liver, tumor, and spleen organs after 1.0 mg/kg IV injection of Peripheral LNPs.

### DETAILED DESCRIPTION

The present disclosure relates to cationic and/or ionizable lipids and lipid-containing particles comprising the same. The disclosure also relates to methods of delivering a therapeutic agent (such as a nucleic acid) to a mammalian cell, methods of producing a polypeptide of interest in a mammalian cell, and methods of treating a disease or disorder in a mammal in need thereof. For example, a method of producing a polypeptide of interest in a cell can comprise a step of contacting a lipid-containing particle described herein with the cell, thereby delivering an mRNA that encodes the polypeptide of interest into the cell, and thereby the mRNA can be translated to produce the polypeptide of interest. For another example, a method of delivering a therapeutic agent to a mammalian cell or organ may involve administration of a lipid-containing particle comprising the therapeutic agent to a subject, in which the administration comprises contacting the cell or organ with the lipid-containing particle, whereby the therapeutic agent is delivered to the cell or organ. In some embodiments, the particle comprises a compound described herein. In some embodiments, the particle is the compound described herein. In some embodiments, the compound is any one of Compounds 1-54.

Described herein, in some aspects, is a compound comprising Formula I′:

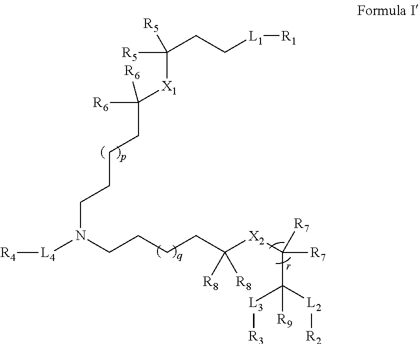

or its N-oxide, or a pharmaceutically acceptable salt thereof. In some embodiments, L₁ is substituted or unsubstituted linear —C₃₋₁₂ alkylene or —C₃₋₁₂ alkenylalkylene. In some embodiments. L₂ is substituted or unsubstituted linear —C₄₋₁₂ alkylene or —C₃₋₁₂ alkenylalkylene. In some embodiments, L₃ is substituted or unsubstituted linear —C₄₋₁₂ alkylene or —C₃₋₁₂ alkenylalkylene. In some embodiments, if L₁, L₂, or L₃ is substituted, then L₁, L₂, or L₃ is substituted with 1-5 substituents selected from the group consisting of halogen, deuterium, —CN, linear or branched C₁₋₁₀ alkyl, linear or branched C₁₋₁₀ heteroalkyl, C₁₋₂ haloalkyl, —C(═O)R¹⁰, —C(═O)N(R¹⁰)₂, —OR¹⁰, —N(R¹⁰)₂, substituted or unsubstituted C₃₋₆ cycloalkyl, and substituted or unsubstituted 3- to 10-membered heterocycloalkyl; wherein each substituted C₃₋₆ cycloalkyl and substituted 3- to 10-membered heterocycloalkyl is substituted with 1-5 R¹¹. In some embodiments, L₄ is substituted or unsubstituted —C₁₋₂₄ alkylene or —C₃₋₂₄ alkenylalkylene; where L₄ is optionally substituted with 1 to 10 R¹¹. In some embodiments, X₁ is —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—. In some embodiments, X₂ is —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—. In some embodiments, each R₁, R₂, and R₃, is independently hydrogen, deuterium, halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl. In some embodiments, at least one of R₁, R₂, and R₃ is not hydrogen or deuterium. In some embodiments, when each R₁, R₂, and R₃ is hydrogen, or deuterium, then at least one of L₁, L₂, L₃ is substituted with 1-5 substituents, and at least one of the substituents is not a linear C₁₋₁₀ alkyl substituted at a terminal carbon, or a deuterium. In some embodiments, R₄ is —OH, —OR¹⁰, —O—C(═O)—R¹⁰, —C(═O)—OR¹⁰, —O—C(═O)—OR¹⁰—, —C(═O)—O—C(═O)—R¹⁰, —O—C(═O)—C₀₋₉ alkylene-R¹¹, —(O—C₀₋₉ alkylene-)ₙR¹¹, —O—C(═O)—C₀₋₉ alkylene-N(R¹⁰)₂, —N(R¹⁰)₂, or —C(═O)—C₀₋₉ alkylene-N(R¹⁰)₂. In some embodiments, each R₅, R₆, R₇, R₈, or R₉ is independently hydrogen, deuterium, halogen, —CN, —OR¹⁰, —N(R¹⁰)₂, —O—C(═O)—R¹⁰, C₁₋₁₀ alkyl, C₁₋₁₀ heteroalkyl, C₃₋₁₀ cycloalkyl, C₃₋₁₀ heterocycloalkyl, C₂₋₆ alkenyl, or C₂₋₆ alkynyl. In some embodiments, each R¹⁰ is independently hydrogen, C₁₋₁₀ alkyl, C₁₋₁₀ heteroalkyl, C₁₋₁₀ haloalkyl, C₃₋₁₀ cycloalkyl, C₃₋₁₀ heterocycloalkyl, C₂₋₆ alkenyl, or C₂₋₆ alkynyl. In some embodiments, each R¹¹ is independently hydrogen, deuterium, halogen, —CN, C₁₋₁₀ alkyl, C₁₋₁₀ heteroalkyl, C₁₋₁₀ haloalkyl, C₃₋₁₀ cycloalkyl, C₃₋₁₀ heterocycloalkyl, C₂₋₆ alkenyl, C₂₋₆ alkynyl, —C(O)OH, —C(O)—O—C₁₋₆ alkyl, —C(O)—C₁₋₆ alkyl, —C(O)NH₂, —C(O)NH(C₁₋₆ alkyl), —C(O)N(C₁₋₆ alkyl)₂, —NH₂, —NH(C₁₋₆ alkyl), —N(C₁₋₆ alkyl)₂, —OH, —O—C₁₋₆ alkyl, —SH, —S(C₁₋₆ alkyl), —S(O)(C₁₋₆ alkyl), —S(O)₂(C₁₋₆ alkyl), or —S(O)₂NH(C₁₋₆ alkyl). In some embodiments, n is an integer from 0-20. In some embodiments, p is 0, 1, 2, 3, 4, or 5. In some embodiments, q is 0, 1, 2, 3, 4, or 5. In some embodiments, r is 0 or 1.

In some aspects, described herein is a compound of Formula I′-a:

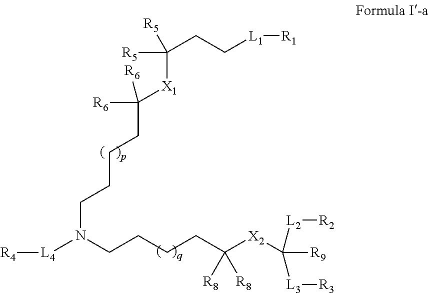

or its N-oxide, or a pharmaceutically acceptable salt thereof. In some embodiments, L₁ is substituted or unsubstituted linear —C₃₋₁₂ alkylene or —C₃₋₁₂ alkenylalkylene. In some embodiments, L₂ is substituted or unsubstituted linear —C₄₋₁₂ alkylene or —C₃₋₁₂ alkenylalkylene. In some embodiments. L₃ is substituted or unsubstituted linear —C₄₋₁₂ alkylene or —C₃₋₁₂ alkenylalkylene. In some embodiments, if L₁, L₂, or L₃ is substituted, then L₁, L₂, or L₃ is substituted with 1-5 substituents selected from the group consisting of halogen, deuterium, —CN, linear or branched C₁₋₁₀ alkyl, linear or branched C₁₋₁₀ heteroalkyl, C₁₋₂ haloalkyl, —C(═O)R¹⁰, —C(═O)N(R¹⁰)₂, —OR¹⁰, —N(R¹⁰)₂, substituted or unsubstituted C₃₋₆ cycloalkyl, and substituted or unsubstituted 3- to 10-membered heterocycloalkyl; wherein each substituted C₃₋₆ cycloalkyl and substituted 3- to 10-membered heterocycloalkyl is substituted with 1-5 R¹¹. In some embodiments, L₄ is substituted or unsubstituted —C₁₋₂₄ alkylene or —C₃₋₂₄ alkenylalkylene; where L₄ is optionally substituted with 1 to 10 R¹¹. In some embodiments, X₁ is —C(═O)—O—, —O—C(═O)—, —O—C(═O)—O—, —C(═O)—N(R¹⁰C)—, —N(R¹⁰)—C(═O)—, S—S, —C(═O)—S—, or —C(C═S)—O—. In some embodiments, X₂ is —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—. In some embodiments, each R₁, R₂, and R₃, is independently hydrogen, deuterium, halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl. In some embodiments, at least one of R₁, R₂, and R₃ is not hydrogen or deuterium. In some embodiments, when each R₁, R₂, and R₃ is hydrogen, or deuterium, then at least one of L₁, L₂, L₃ is substituted with 1-5 substituents, and at least one of the substituents is not a linear C₁₋₁₀ alkyl substituted at a terminal carbon, or a deuterium. In some embodiments, R₄ is —OH, —OR¹⁰, —O—C(═O)—R¹⁰, —C(═O)—OR¹⁰, —O—C(═O)—OR¹⁰—, —C(═O)—O—C(═O)—R¹⁰, —O—C(═O)—C₀₋₉ alkylene-R¹¹, —(O—C₀₋₉ alkylene-)ₙR¹¹, —O—C(═O)—C₀₋₉ alkylene-N(R¹⁰)₂, —N(R¹⁰)₂, or —C(═O)—C₀₋₉ alkylene-N(R¹⁰)₂. In some embodiments, each R₅, R₆, R₈, or R₉ is independently hydrogen, deuterium, halogen, —CN, —OR¹⁰, —N(R¹⁰)₂, —O—C(═O)—R¹⁰, C₁₋₁₀ alkyl, C₁₋₁₀ heteroalkyl, C₃₋₁₀ cycloalkyl, C₃₋₁₀ heterocycloalkyl, C₂₋₆ alkenyl, or C₂₋₆ alkynyl. In some embodiments, each R¹⁰ is independently hydrogen, C₁₋₁₀ alkyl, C₁₋₁₀ heteroalkyl, C₁₋₁₀ haloalkyl, C₃₋₁₀ cycloalkyl, C₃₋₁₀ heterocycloalkyl, C₂₋₆ alkenyl, or C₂₋₆ alkynyl. In some embodiments, each R¹¹ is independently hydrogen, deuterium, halogen, —CN, C₁₋₁₀ alkyl, C₁₋₁₀ heteroalkyl, C₁₋₁₀ haloalkyl, C₃₋₁₀ cycloalkyl, C₃₋₁₀ heterocycloalkyl, C₂₋₆ alkenyl, C₂₋₆ alkynyl, —C(O)OH, —C(O)—O—C₁₋₆ alkyl, —C(O)—C₁₋₆ alkyl, —C(O)NH₂, —C(O)NH(C₁₋₆ alkyl), —C(O)N(C₁₋₆ alkyl)₂, —NH₂, —NH(C₁₋₆ alkyl), —N(C₁₋₆ alkyl)₂, —OH, —O—C₁₋₆ alkyl, —SH, —S(C₁₋₆ alkyl), —S(O)(C₁₋₆ alkyl), —S(O)₂(C₁₋₆ alkyl), or —S(O)₂NH(C₁₋₆ alkyl). In some embodiments, n is an integer from 0-20. In some embodiments, p is 0, 1, 2, 3, 4, or 5. In some embodiments, q is 0, 1, 2, 3, 4, or 5.

Described herein, in some aspects, is a compound comprising Formula I:

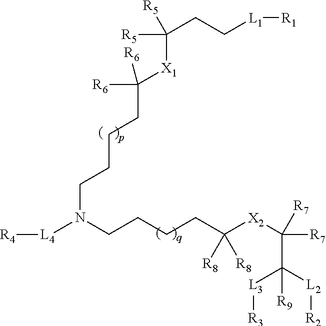

or its N-oxide, or a pharmaceutically acceptable salt thereof. In some embodiments, L₁ is substituted or unsubstituted linear —C₃₋₁₂ alkylene or —C₃₋₁₂ alkenylalkylene. In some embodiments, L₂ is substituted or unsubstituted linear —C₄₋₁₂ alkylene or —C₃₋₁₂ alkenylalkylene. In some embodiments, L₃ is substituted or unsubstituted linear —C₄₋₁₂ alkylene or —C₃₋₁₂ alkenylalkylene. In some embodiments, if L₁, L₂, or L₃ is substituted, then L₁, L₂, or La is substituted with 1-5 substituents selected from the group consisting of halogen, deuterium, —CN, linear or branched C₁₋₁₀ alkyl, linear or branched C₁₋₁₀ heteroalkyl, C₁₋₂ haloalkyl, —C(═O)R¹⁰, —C(═O)N(R¹⁰)₂, —OR¹⁰, —N(R¹⁰)₂, substituted or unsubstituted C₃₋₆ cycloalkyl, and substituted or unsubstituted 3- to 10-membered heterocycloalkyl; wherein each substituted C₃₋₆ cycloalkyl and substituted 3- to 10-membered heterocycloalkyl is substituted with 1-5 R¹¹. In some embodiments, L₄ is substituted or unsubstituted —C₁₋₂₄ alkylene or —C₃₋₂₄ alkenylalkylene; where L₄ is optionally substituted with 1 to 10 R¹¹. In some embodiments, X₁ is —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—. In some embodiments, X₂ is —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—. In some embodiments, each R₁, R₂, and R₃, is independently hydrogen, deuterium, halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl. In some embodiments, at least one of R₁, R₂, and R₃ is not hydrogen or deuterium. In some embodiments, when each R₁, R₂, and R₃ is hydrogen, or deuterium, then at least one of L₁, L₂, L₃ is substituted with 1-5 substituents, and at least one of the substituents is not a linear C₁₋₁₀ alkyl substituted at a terminal carbon, or a deuterium. In some embodiments, R₄ is —OH, —OR¹⁰, —O—C(═O)—R¹⁰, —C(═O)—OR¹⁰, —O—C(═O)—OR¹⁰—, —C(═O)—O—C(═O)—R¹⁰, —O—C(═O)—C₀₋₉ alkylene-R¹¹, —(O—C₀₋₉ alkylene-)R¹¹, —O—C(═O)—C₀₋₉ alkylene-N(R¹⁰)₂, —N(R¹⁰)₂, or —C(═O)—C₀₋₉ alkylene-N(R¹⁰)₂. In some embodiments, each R₅, R₆, R₇, R₈, or R₉ is independently hydrogen, deuterium, halogen, —CN, —OR¹⁰, —N(R¹⁰)₂, —O—C(═O)—R¹⁰, C₁₋₁₀ alkyl, C₁₋₁₀ heteroalkyl, C₃₋₁₀ cycloalkyl, C₃₋₁₀ heterocycloalkyl, C₂₋₆ alkenyl, or C₂₋₆ alkynyl. In some embodiments, each R¹⁰ is independently hydrogen, C₁₋₁₀ alkyl, C₁₋₁₀ heteroalkyl, C₁₋₁₀ haloalkyl, C₃₋₁₀ cycloalkyl, C₃₋₁₀ heterocycloalkyl, C₂₋₆ alkenyl, or C₂₋₆ alkynyl. In some embodiments, each R¹¹ is independently hydrogen, deuterium, halogen, —CN, C₁₋₁₀ alkyl, C₁₋₁₀ heteroalkyl, C₁₋₁₀ haloalkyl, C₃₋₁₀ cycloalkyl, C₃₋₁₀ heterocycloalkyl, C₂₋₆ alkenyl, C₂₋₆ alkynyl, —C(O)OH, —C(O)—O—C₁₋₆ alkyl, —C(O)—C₁₋₆ alkyl, —C(O)NH₂, —C(O)NH(C₁₋₆ alkyl), —C(O)N(C₁₋₆ alkyl)₂, —NH₂, —NH(C₁₋₆ alkyl), —N(C₁₋₆ alkyl)₂, —OH, —O—C₁₋₆ alkyl, —SH, —S(C₁₋₆ alkyl), —S(O)(C₁₋₆ alkyl), —S(O)₂(C₁₋₆ alkyl), or —S(O)₂NH(C₁₋₆ alkyl). In some embodiments, n is an integer from 0-20. In some embodiments, p is 0, 1, 2, 3, 4, or 5. In some embodiments, q is 0, 1, 2, 3, 4, or 5.

Described herein, in some aspects, is a compound comprising a structure of Formula II:

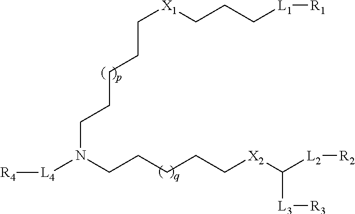

or its N-oxide, or a pharmaceutically acceptable salt thereof. In some embodiments, L₁ is substituted or unsubstituted linear —C₃₋₁₂ alkylene. In some embodiments. L₂ is substituted or unsubstituted linear —C₄₋₁₂ alkylene. In some embodiments, L₃ is substituted or unsubstituted linear —C₄₋₁₂ alkylene. In some embodiments, if L₁, L₂, or L₃ is substituted, then L₁, L₂, or L₃ is substituted with 1-5 substituents selected from the group consisting of halogen, deuterium, —CN, linear or branched C₁₋₁₀ alkyl, linear or branched C₁₋₁₀ heteroalkyl, C₁₋₂ haloalkyl, —C(═O)R¹⁰, —C(═O)N(R¹⁰)₂, —OR¹⁰, —N(R¹⁰)₂, substituted or unsubstituted C₃₋₆ cycloalkyl, and substituted or unsubstituted 3- to 10-membered heterocycloalkyl; wherein each substituted C₃₋₆ cycloalkyl and substituted 3- to 10-membered heterocycloalkyl is substituted with 1-5 R¹¹. In some embodiments, L₄ is substituted or unsubstituted —C₁₋₂₄ alkylene. In some embodiments, L₄ is optionally substituted with 1 to 10 R¹¹. In some embodiments, X₁ is —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—. In some embodiments, X₂ is —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—. In some embodiments, each R₁, R₂, and R₃, is independently hydrogen, deuterium, halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl, provided that at least one of R₁, R₂, and R₃ is not hydrogen or deuterium or when each R₁, R₂, and R₃ is hydrogen, or deuterium, then at least one of L₁, L₂, L₃ is substituted with 1-5 substituents, and at least one of the substituents is not a linear C₁₋₁₀ alkyl substituted at a terminal carbon, or a deuterium. In some embodiments, R₄ is —OH, —OR¹⁰, —O—C(═O)—R¹⁰, —C(═O)—OR¹⁰, —O—C(═O)—OR¹⁰—, —C(═O)—O—C(═O)—R¹⁰, —O—C(═O)—C₀₋₉ alkylene-R¹¹, —(O—C₀₋₉ alkylene-)ₙR¹¹, —O—C(═O)—C₀₋₉ alkylene-N(R¹⁰)₂, —N(R¹⁰)₂, or —C(═O)—C₀₋₉ alkylene-N(R¹⁰)₂. In some embodiments, each R₅, R₆, R₇, R₈, or R₉ is independently hydrogen, deuterium, halogen, —CN, —OR¹⁰, —N(R¹⁰)₂, —O—C(═O)—R¹⁰, C₁₋₁₀ alkyl, C₁₋₁₀ heteroalkyl, C₃₋₁₀ cycloalkyl, C₃₋₁₀ heterocycloalkyl, C₂₋₆ alkenyl, or C₂₋₆ alkynyl. In some embodiments, each R¹⁰ is independently hydrogen, C₁₋₁₀ alkyl, C₁₋₁₀ heteroalkyl, C₁₋₁₀ haloalkyl, C₃₋₁₀ cycloalkyl, C₃₋₁₀ heterocycloalkyl, C₂₋₆ alkenyl, or C₂₋₆ alkynyl. In some embodiments, each R¹¹ is independently hydrogen, deuterium, halogen, —CN, C₁₋₁₀ alkyl, C₁₋₁₀ heteroalkyl, C₁₋₁₀ haloalkyl, C₃₋₁₀ cycloalkyl, C₃₋₁₀ heterocycloalkyl, C₂₋₆ alkenyl, C₂₋₆ alkynyl, —C(O)OH, —C(O)—O—C₁₋₆ alkyl, —C(O)—C₁₋₆ alkyl, —C(O)NH₂, —C(O)NH(C₁₋₆ alkyl), —C(O)N(C₁₋₆ alkyl)₂, —NH₂, —NH(C₁₋₆ alkyl), —N(C₁₋₆ alkyl)₂, —OH, —O—C₁₋₆ alkyl, —SH, —S(C₁₋₆ alkyl), —S(O)(C₁₋₆ alkyl), —S(O)₂(C₁₋₆ alkyl), or —S(O)₂NH(C₁₋₆ alkyl). In some embodiments, n is an integer from 0-20. In some embodiments, p is 0, 1, 2, 3, 4, or 5. In some embodiments, q is 0, 1, 2, 3, 4, or 5.

In some embodiments, the compound comprises a structure of Formula III:

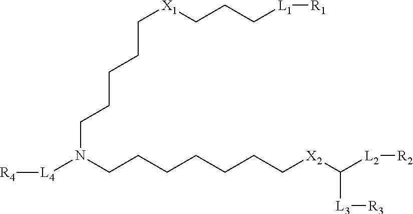

or its N-oxide, or a pharmaceutically acceptable salt thereof. In some embodiments, the compound comprises a structure of Formula III-a:

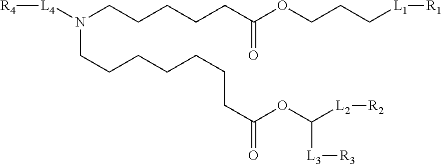

or its N-oxide, or a pharmaceutically acceptable salt thereof. In some embodiments, the compound comprises a structure of Formula IV:

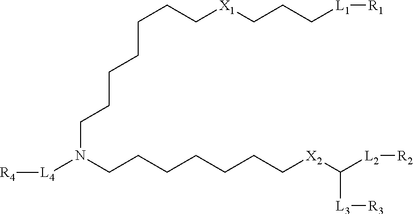

or its N-oxide, or a pharmaceutically acceptable salt thereof. In some embodiments, the compound comprises a structure of Formula IV-a:

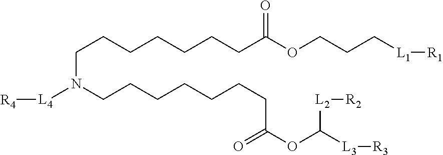

or its N-oxide, or a pharmaceutically acceptable salt thereof.

In some embodiments, described herein is a compound of a compound of Formula V:

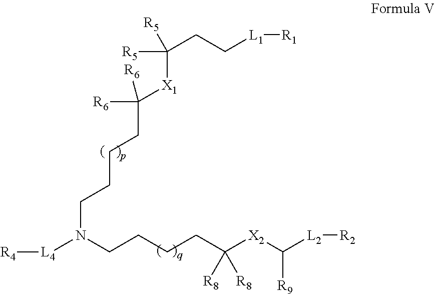

or its N-oxide, or a pharmaceutically acceptable salt thereof. In some embodiments, L₁ is substituted or unsubstituted linear —C₃₋₁₂ alkylene or —C₃₋₁₂ alkenylalkylene. In some embodiments, L₂ is substituted or unsubstituted linear —C₄₋₁₂ alkylene or —C₃₋₁₂ alkenylalkylene. In some embodiments, L₃ is substituted or unsubstituted linear —C₄₋₁₂ alkylene or —C₃₋₁₂ alkenylalkylene. In some embodiments, if L₁, L₂, or L₃ is substituted, then L₁, L₂, or L₃ is substituted with 1-5 substituents selected from the group consisting of halogen, deuterium, —CN, linear or branched C₁₋₁₀ alkyl, linear or branched C₁₋₁₀ heteroalkyl, C₁₋₂ haloalkyl, —C(═O)R¹⁰, —C(═O)N(R¹⁰)₂, —OR¹⁰, —N(R¹⁰)₂, substituted or unsubstituted C₃₋₆ cycloalkyl, and substituted or unsubstituted 3- to 10-membered heterocycloalkyl; wherein each substituted C₃₋₆ cycloalkyl and substituted 3- to 10-membered heterocycloalkyl is substituted with 1-5 R¹¹. In some embodiments, L₄ is substituted or unsubstituted —C₁₋₂₄ alkylene or —C₃₋₂₄ alkenylalkylene; where L₄ is optionally substituted with 1 to 10 R¹¹. In some embodiments, X₁ is —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—. In some embodiments, X₂ is —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—. In some embodiments, each R₁, R₂, and R₃, is independently hydrogen, deuterium, halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl. In some embodiments, at least one of R₁, R₂, and R₃ is not hydrogen or deuterium. In some embodiments, when each R₁, R₂, and R₃ is hydrogen, or deuterium, then at least one of L₁, L₂, L₃ is substituted with 1-5 substituents, and at least one of the substituents is not a linear C₁₋₁₀ alkyl substituted at a terminal carbon, or a deuterium. In some embodiments, R₄ is —OH, —OR¹⁰, —O—C(═O)—R¹⁰, —C(═O)—OR¹⁰, —O—C(═O)—OR¹⁰—, —C(═O)—O—C(═O)—R¹⁰, —O—C(═O)—C₀₋₉ alkylene-R¹¹, —(O)—C₀₋₉ alkylene-)ₙR¹¹, —O—C(═O)—C₀₋₉ alkylene-N(R¹⁰)₂, —N(R¹⁰)₂, or —C(═O)—C₀₋₉ alkylene-N(R¹⁰)₂. In some embodiments, each R₅, R₆, R₇, R₈, or R₉ is independently hydrogen, deuterium, halogen, —CN, —OR¹⁰, —N(R¹⁰)₂, —O—C(═O)—R¹⁰, C₁₋₁₀ alkyl, C₁₋₁₀ heteroalkyl, C₃₋₁₀ cycloalkyl, C₃₋₁₀ heterocycloalkyl, C₂₋₆ alkenyl, or C₂₋₆ alkynyl. In some embodiments, each R¹⁰ is independently hydrogen, C₁₋₁₀ alkyl, C₁₋₁₀ heteroalkyl, C₁₋₁₀ haloalkyl, C₃₋₁₀ cycloalkyl, C₃₋₁₀ heterocycloalkyl, C₂₋₆ alkenyl, or C₂₋₆ alkynyl. In some embodiments, each R¹¹ is independently hydrogen, deuterium, halogen, —CN, C₁₋₁₀ alkyl, C₁₋₁₀ heteroalkyl, C₁₋₁₀ haloalkyl, C₃₋₁₀ cycloalkyl, C₃₋₁₀ heterocycloalkyl, C₂₋₆ alkenyl, C₂₋₆ alkynyl, —C(O)OH, —C(O)—O—C₁₋₆ alkyl, —C(O)—C₁₋₆ alkyl, —C(O)NH₂, —C(O)NH(C₁₋₆ alkyl), —C(O)N(C₁₋₆ alkyl)₂, —NH₂, —NH(C₁₋₆ alkyl), —N(C₁₋₆ alkyl)₂, —OH, —O—C₁₋₆ alkyl, —SH, —S(C₁₋₆ alkyl), —S(O)(C₁₋₆ alkyl), —S(O)₂(C₁₋₆ alkyl), or —S(O)₂NH(C₁₋₆ alkyl). In some embodiments, n is an integer from 0-20. In some embodiments, p is 0, 1, 2, 3, 4, or 5. In some embodiments, q is 0, 1, 2, 3, 4, or 5.

In some embodiments, the compound comprises a structure, or its N-oxide, or a pharmaceutically acceptable salt thereof of:

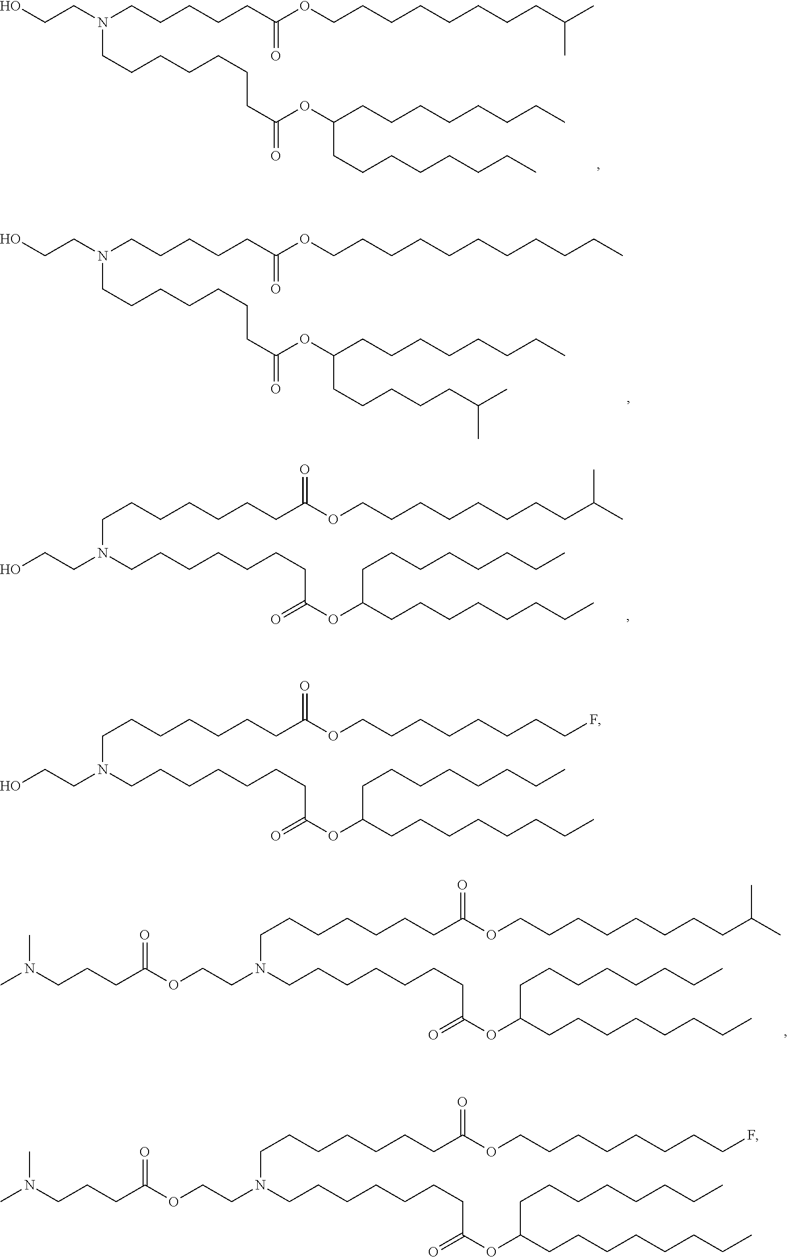

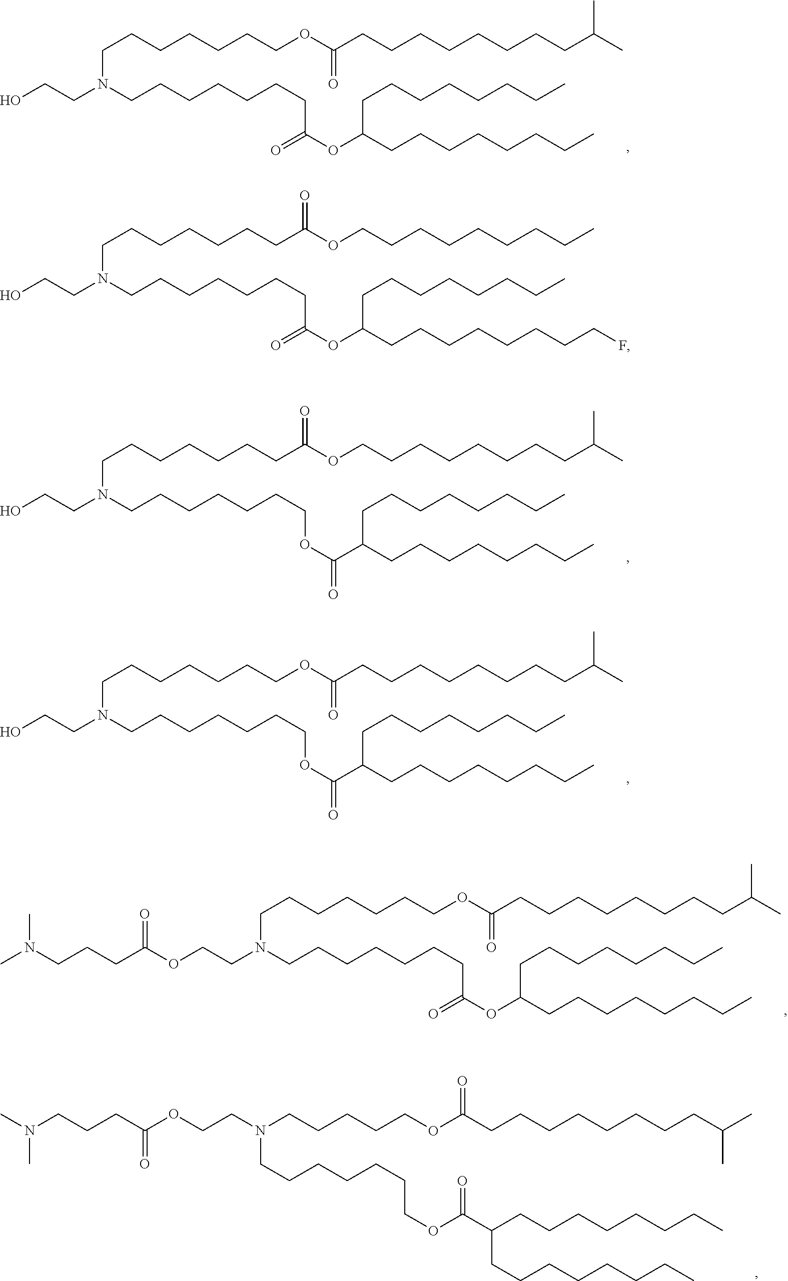

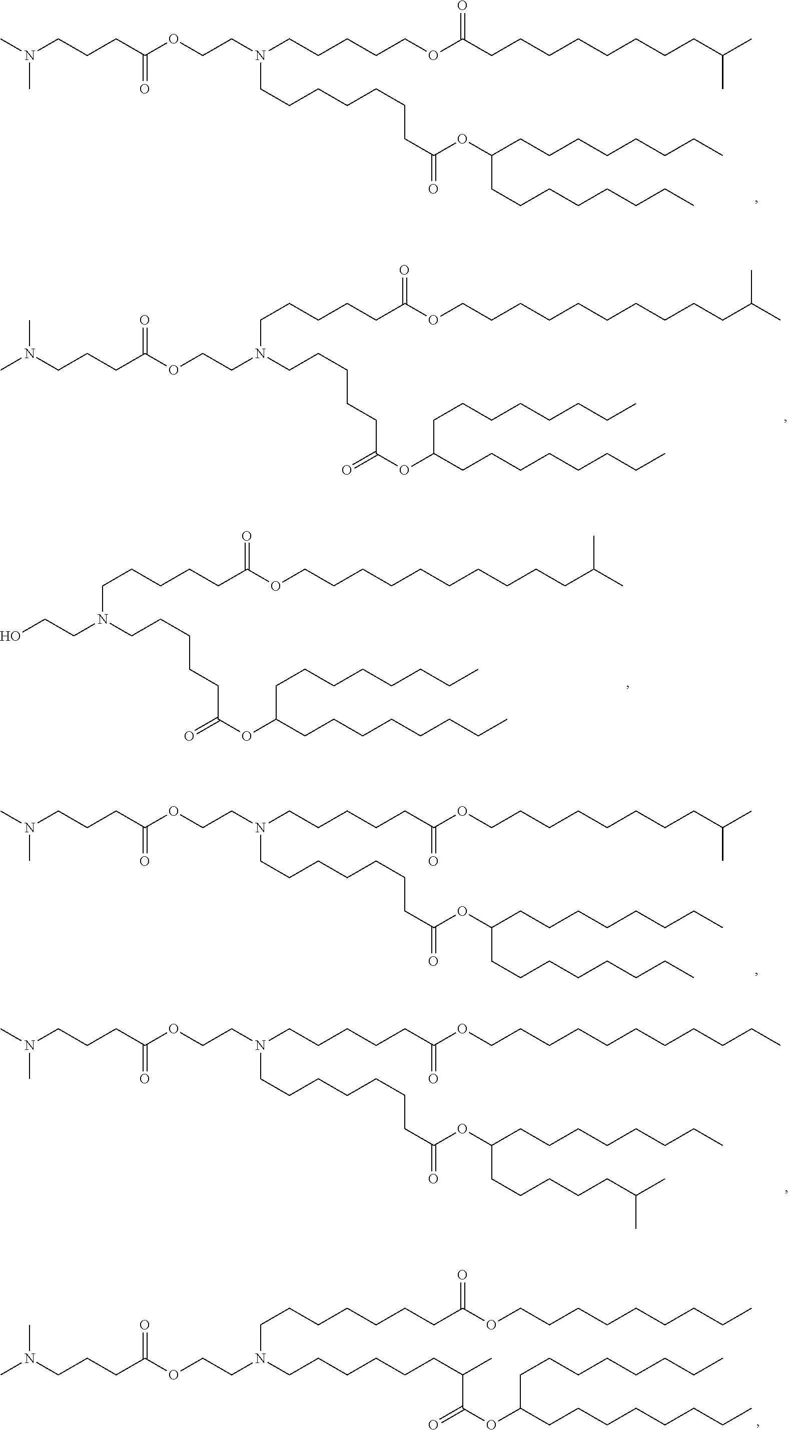

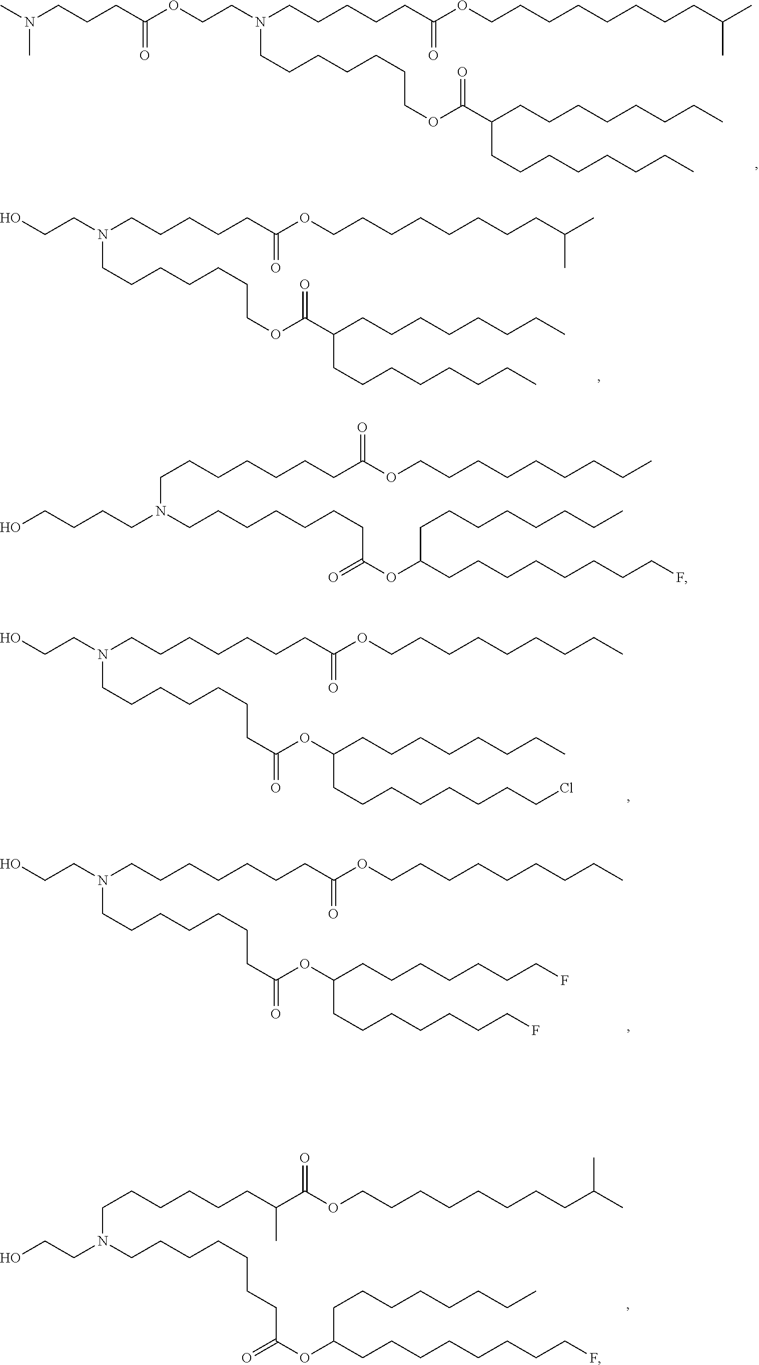

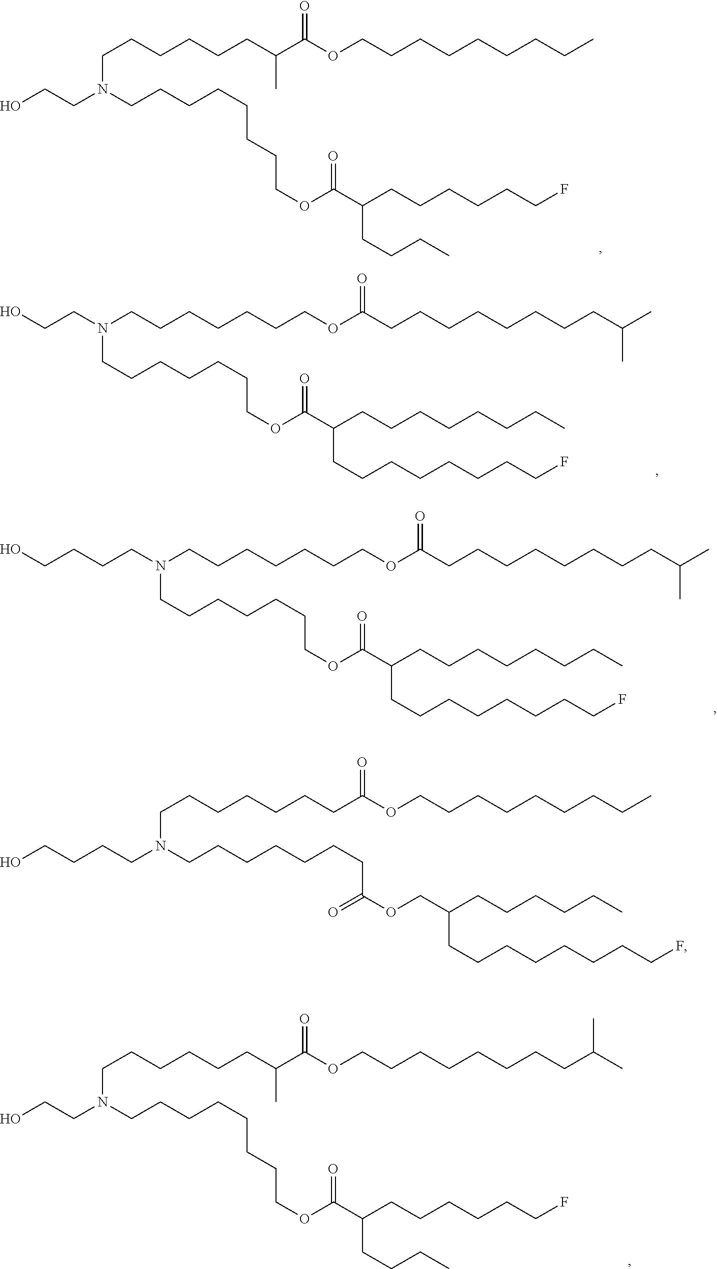

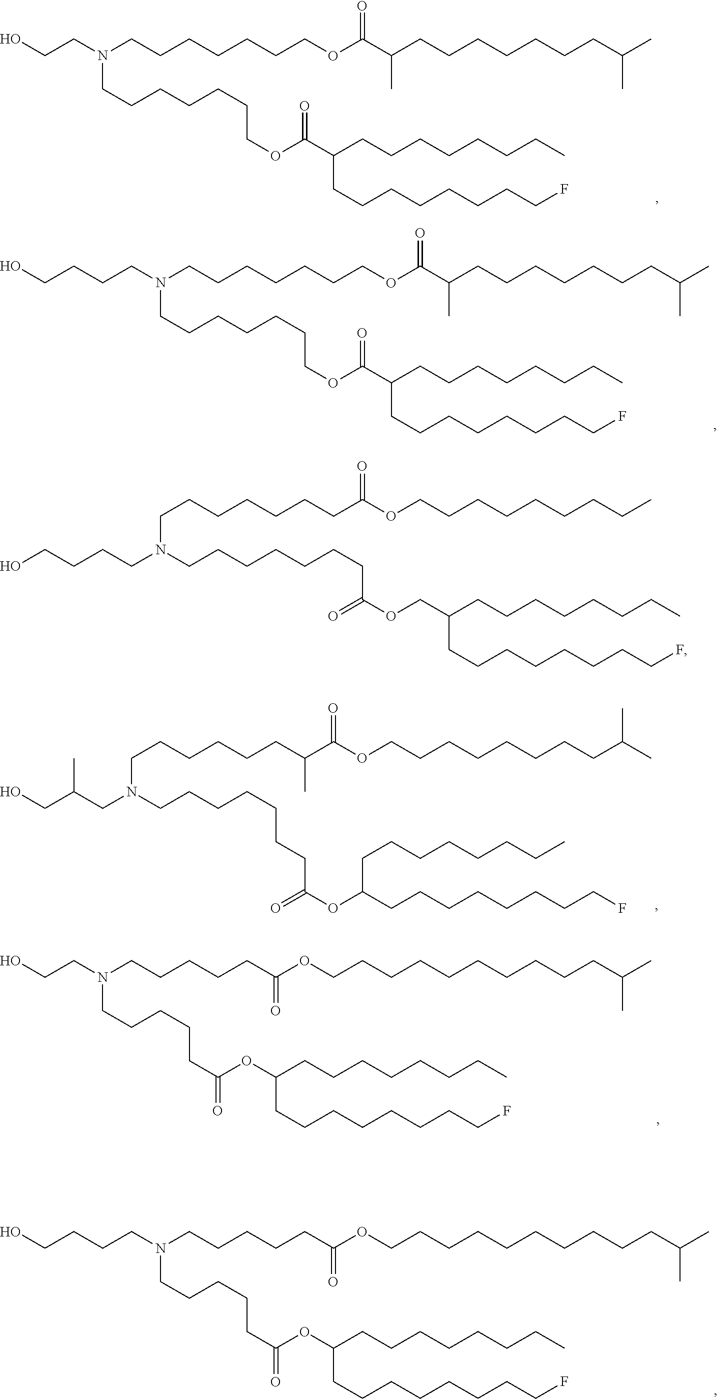

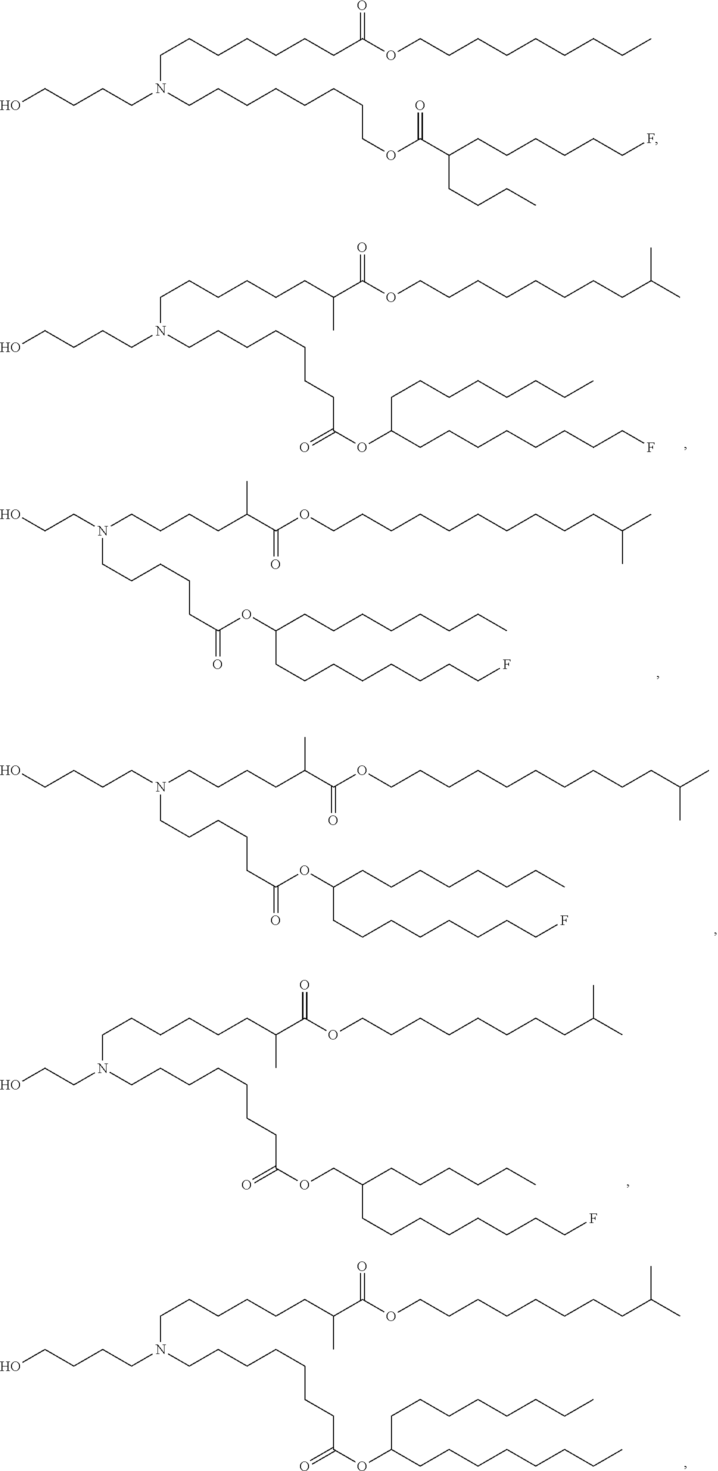

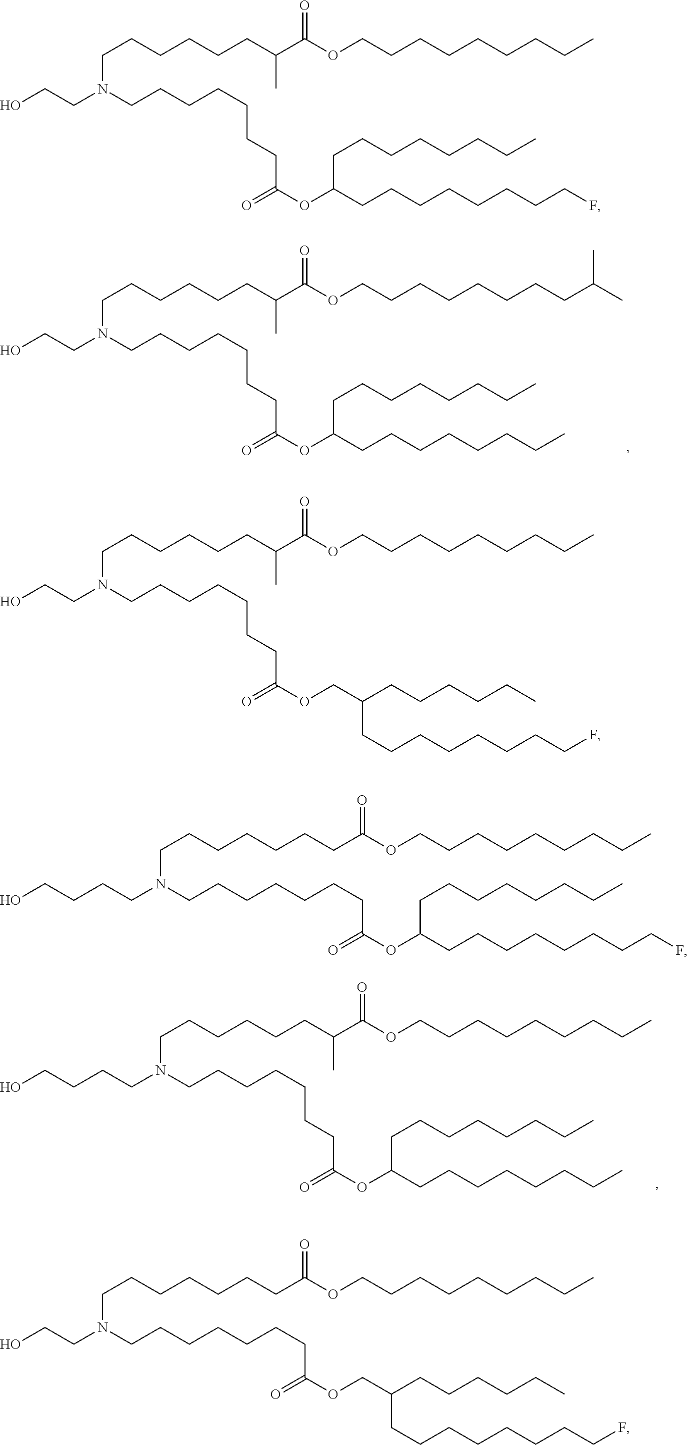

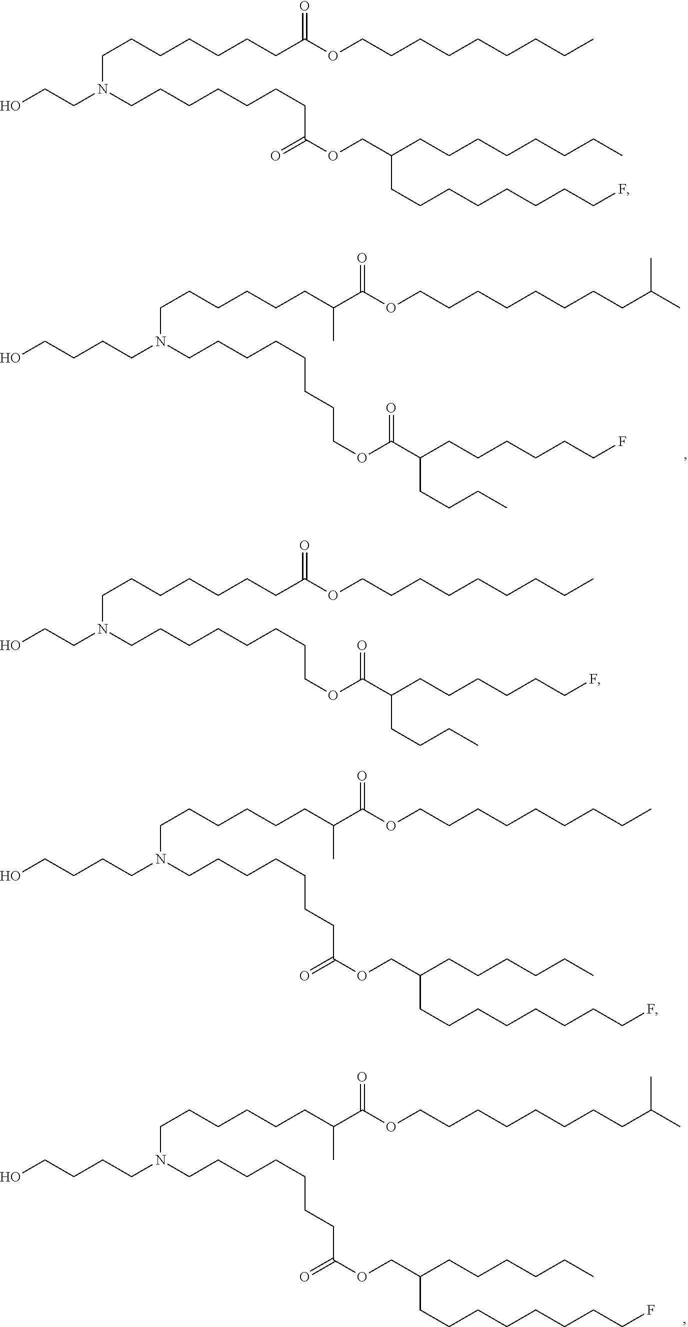 [SMILES: CCCCCCCCCOC(=O)CCCCCCCN(CCO)CCCCCCCC(=O)OCC(CCCCCCCC)CCCCCCCCF ; CCCCC(CCCCCCF)C(=O)OCCCCCCCCN(CCCCO)CCCCCCC(C)C(=O)OCCCCCCCCC(C)C ; CCCCCCCCCOC(=O)CCCCCCCN(CCO)CCCCCCCCOC(=O)C(CCCC)CCCCCCF ; CCCCCCCCCOC(=O)C(C)CCCCCCN(CCCCO)CCCCCCCC(=O)OCC(CCCCCC)CCCCCCCCF ; CCCCCCC(CCCCCCCCF)COC(=O)CCCCCCCN(CCCCO)CCCCCCC(C)C(=O)OCCCCCCCCC(C)C]

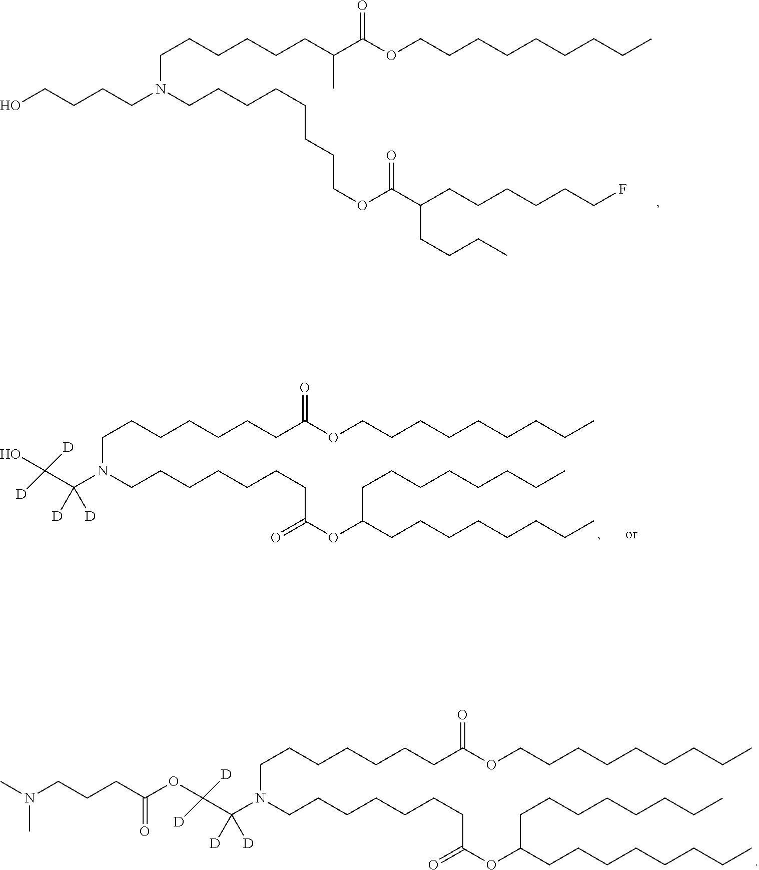

Described herein, in some aspects, is a nanoparticle composition comprising a compound described herein and a lipid component. In some embodiments, the lipid component comprises a structural lipid such as cholesterol, fecosterol, sitosterol, ergosterol, campesterol, stigmasterol, brassicasterol, tomatidine, ursolic acid, alpha-tocopherol, or a combination thereof. In some embodiments, the nanoparticle composition comprises a polyethylene glycol (PEG) lipid. In some embodiments, the PEG lipid comprises a PEG-modified phosphatidy-lethanolamine, a PEG-modified phosphatidic acid, a PEG-modified ceramide, a PEG-modified dialkylamine, a PEG-modified diacylglycerol, a PEG-modified dialkylglycerol, or a combination thereof. In some embodiments, the lipid component further comprises a cationic and/or ionizable lipid. In some embodiments, the nanoparticle composition further comprises a therapeutic and/or prophylactic agent. In some embodiments, the therapeutic and/or prophylactic agent is nucleic acid. In some embodiments, the therapeutic and/or prophylactic agent is ribonucleic acid (RNA). In some embodiments, the RNA is a small interfering RNA (siRNA), an asymmetrical interfering RNA (aiRNA), a microRNA (miRNA), a Dicer-substrate RNA (dsRNA), a small hairpin RNA (shRNA), or a messenger RNA (mRNA). Om some embodiments, the RNA is mRNA. In some embodiments, the mRNA encodes a polypeptide associated with immune response. For example, the mRNA can encode a polypeptide comprising an interleukin or an interferon.

Described herein, in some aspects, are methods for contacting a cell with a compound described herein or a nanoparticle composition described herein, where the compound described herein or the nanoparticle composition described herein delivers a nucleic acid described herein to the cell. In some embodiments, the method comprises treating a disease or condition associated with the cell. In some embodiments, the method comprises expressing the nucleic acid, where the expression of the nucleic acid results into a polypeptide for treating the disease or condition.

### Nanoparticle Composition

Provided herein in some embodiments are LNP compositions comprising an amino lipid, a phospholipid, a PEG-lipid, a cholesterol or a derivative thereof, a payload, or any combination thereof useful for delivery of therapeutic agents to mammalian cells or organs.

### Amino Lipids

Some embodiments described herein include amino lipid compounds having the structure of Formula (I′):

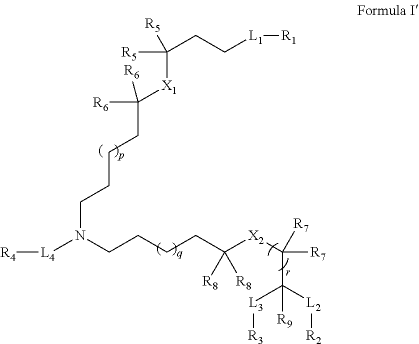

or its N-oxide, or a pharmaceutically acceptable salt thereof,
wherein:
L₁ is substituted or unsubstituted linear —C₃₋₁₂ alkylene or —C₃₋₁₂ alkenylalkylene;
L₂ is substituted or unsubstituted linear —C₄₋₁₂ alkylene or —C₃₋₁₂ alkenylalkylene;
L₃ is substituted or unsubstituted linear —C₄₋₁₂ alkylene or —C₃₋₁₂ alkenylalkylene;
wherein if L₁, L₂, or L₃ is substituted, then L₁, L₂, or L₃ is substituted with 1-5 substituents selected from the group consisting of halogen, deuterium, —CN, linear or branched C₁₋₁₀ alkyl, linear or branched C₁₋₁₀ heteroalkyl, C₁₋₂ haloalkyl, —C(═O)R¹⁰, —C(═O)N(R¹⁰)₂, —OR¹⁰, —N(R¹⁰)₂, substituted or unsubstituted C₃₋₆ cycloalkyl, and substituted or unsubstituted 3- to 10-membered heterocycloalkyl; wherein each substituted C₃₋₆ cycloalkyl and substituted 3- to 10-membered heterocycloalkyl is substituted with 1-5 R¹¹;
L₄ is substituted or unsubstituted —C₁₋₂₄ alkylene or —C₃₋₂₄ alkenylalkylene; wherein L₄ is optionally substituted with 1 to 10 R¹¹;
X₁ is a covalent bond, —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—;
X₂ is a covalent bond, —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—;
each R₁, R₂, and R₃, is independently hydrogen, deuterium, halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl;
provided that at least one of R₁, R₂, and R₃ is not hydrogen or deuterium;
or when each R₁, R₂, and R₃ is hydrogen, or deuterium, then at least one of L₁, L₂, L₃ is substituted with 1-5 substituents, and at least one of the substituents is not a linear C₁₋₁₀ alkyl substituted at a terminal carbon, or a deuterium;
R₄ is halogen, —OH, —OR¹⁰, —O—C(═O)—R¹⁰, —C(═O)—OR¹⁰, —O—C(═O)—OR¹⁰—, —C(═O)—O—C(═O)—R¹⁰, —O—C(═O)—C₀₋₉ alkylene-R¹¹, —(O—C₀₋₉ alkylene-)ₙR¹¹, —O—C(═O)—C₀₋₉ alkylene-N(R¹⁰)₂, —N(R¹⁰)₂, or —C(═O)—C₀₋₉ alkylene-N(R¹⁰)₂;
each R₅, R₆, R₇, R₈, or R₉ is independently hydrogen, deuterium, halogen, —CN, —OR¹⁰, —N(R¹⁰)₂, —O—C(═O)—R¹⁰, C₁₋₁₀ alkyl, C₁₋₁₀ heteroalkyl, C₃₋₁₀ cycloalkyl, C₃₋₁₀ heterocycloalkyl, C₂₋₆ alkenyl, or C₂₋₆ alkynyl;
each R¹⁰ is independently hydrogen, C₁₋₁₀ alkyl, C₁₋₁₀ heteroalkyl, C₁₋₁₀ haloalkyl, C₃₋₁₀ cycloalkyl, C₃₋₁₀ heterocycloalkyl, C₂₋₆ alkenyl, or C₂₋₆ alkynyl;
each R¹¹ is independently hydrogen, deuterium, halogen, —CN, C₁₋₁₀ alkyl, C₁₋₁₀ heteroalkyl, C₁₋₁₀ haloalkyl, C₃₋₁₀ cycloalkyl, C₃₋₁₀ heterocycloalkyl, C₂₋₆ alkenyl, C₂₋₆ alkynyl, —C(O)OH, —C(O)—O—C₁₋₆ alkyl, —C(O)—C₁₋₆ alkyl, —C(O)NH₂, —C(O)NH(C₁₋₆ alkyl), —C(O)N(C₁₋₆ alkyl)₂, —NH₂, —NH(C₁₋₆ alkyl), —N(C₁₋₆ alkyl)₂, —OH, —O—C₁₋₆ alkyl, —SH, —S(C₁₋₆ alkyl), —S(O)(C₁₋₆ alkyl), —S(O)₂(C₁₋₆ alkyl), or —S(O)₂NH(C₁₋₆ alkyl);
n is an integer from 0-20;
p is 0, 1, 2, 3, 4, or 5;
q is 0, 1, 2, 3, 4, or 5; and
r is 0 or 1.

In some embodiments, r is 0. In some embodiments, r is 1. In some embodiments, X₁ is a covalent bond. In some embodiments, X₂ is a covalent bond. In some embodiments, at least one of X₁ and X₂ is not a covalent bond. In some embodiments, X₁ is a covalent bond and X₂ is —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—. In some embodiments, X₂ is a covalent bond and X₁ is —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—. In some embodiments, X₁ is —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—. In some embodiments, X₂ is —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—. In some embodiments, two R₅ are same. In some embodiments, two R₅ are different. In some embodiments, two R₆ are same. In some embodiments, two R₆ are different. In some embodiments, two R₇ are same. In some embodiments, two R₇ are different. In some embodiments, two R₈ are same. In some embodiments, two R₈ are different. In some embodiments, each R₅, R₆, R₇, R₈, or R₉ is independently hydrogen, deuterium, halogen, —CN, —OR¹⁰, or C₁₋₁₀ alkyl. In some embodiments, each R₅, R₆, R₇, R₈, or R₉ is independently hydrogen, deuterium, halogen, or C₁₋₁₀ alkyl. In some embodiments, each R₅, R₆, R₇, R₈, or R₉ is independently hydrogen, deuterium, halogen, or C₁₋₈ alkyl. In some embodiments, each R₅, R₆, R₇, R₈, or R₉ is independently hydrogen, deuterium, halogen, or C₁₋₆ alkyl. In some embodiments, each R₅, R₆, R₇, R₈, or R₉ is independently hydrogen, deuterium, fluoro, or methyl. In certain embodiments, each R₅, R₆, R₇, R₈, or Rois independently hydrogen, deuterium, halogen, methyl, ethyl, or isopropyl. In some embodiments, each R₅, R₆, R₇, R₈, or R₉ is independently hydrogen or deuterium. In some embodiments, each R₅, R₆, R₇, R₈, or R₉ is independently hydrogen. In certain embodiments, each R₅ is independently hydrogen, halogen or C₁₋₁₀ alkyl, wherein at least one R₅ is not hydrogen, and R₆, R₇, R₈, or R₉ are H. In certain embodiments, each R₅ is independently hydrogen, halogen or C₁₋₈ alkyl, wherein at least one R₅ is not hydrogen, and R₆, R₇, R₈, or R₉ are H. In certain embodiments, each R₅ is independently hydrogen, halogen or C₁₋₆ alkyl, wherein at least one R₅ is not hydrogen, and R₆, R₇, R₈, or R₉ are H. In some embodiments, each R₆ is hydrogen, halogen or C₁₋₆ alkyl, wherein at least one R₆ is not hydrogen, and R₅, R₇, R₈, or R₉ are H. In some embodiments, each R₇ is independently hydrogen, halogen or C₁₋₆ alkyl, wherein at least one R₇ is not hydrogen, and R₅, R₆, R₈, or R₉ are H. In some embodiments, each R₈ is independently hydrogen, halogen or C₁₋₆ alkyl, wherein at least one R₈ is not hydrogen, and R₅, R₆, R₇, or R₉ are H. In some embodiments, R₉ is hydrogen, halogen or C₁₋₆ alkyl, and R₅, R₆, R₇, or R₈ are H. In some embodiments, R₉ is not hydrogen, and R₅, R₆, R₇, or R₈ are H. In some embodiments, R₉ is halogen or C₁₋₆ alkyl, and R₅, R₆, R₇, or R₈ are H. In some embodiments, one of R₅ is methyl and the other R₅ and R₆, R₇, R₈, or R₉ are H. In some embodiments, one of R₅ is ethyl and the other R₅ and R₆, R₇, R₈, or R₉ are H. In some embodiments, one of R₅ is C₃ alkyl and the other R₅ and R₆, R₇, R₈, or R₉ are H. In some embodiments, one of R₅ is C₄ alkyl and the other R₅ and R₆, R₇, R₈, or R₉ are H. In some embodiments, one of R₅ is C₅ alkyl and the other R₅ and R₆, R₇, R₈, or R₉ are H. In some embodiments, one of R₅ is C₆ alkyl and the other R₅ and R₆, R₇, R₈, or R₉ are H. In some embodiments, one of R₅ is C₇ alkyl and the other R₅ and R₆, R₇, R₅, or R₉ are H. In some embodiments, one of R₅ is C₈ alkyl and the other R₅ and R₆, R₇, R₈, or R₉ are H. In some embodiments, one of R₅ is C₉ alkyl and the other R₅ and R₆, R₇, R₈, or R₉ are H. In some embodiments, one of R₅ is C₁₀ alkyl and the other R₅ and R₆, R₇, R₈, or R₉ are H. In some embodiments, one of R₅ is

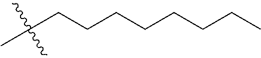 [SMILES: *CCCCCCCC]

and the other R₅ and R₆, R₇, R₈, or R₉ are H. In some embodiments, one of R₅ is

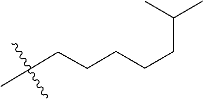 [SMILES: *CCCCCC(C)C]

and the other R₅ and R₆, R₇, R₈, or R₉ are H. In some embodiments, one of R₆ is methyl and the other R₆ and R₅, R₇, R₈, or R₉ are H. In some embodiments, one of R₇ is methyl and the other R₇ and R₅, R₆, R₈, or R₉ are H. In some embodiments, one of R₈ is methyl and the other R₈ and R₅, R₆, R₇, or R₉ are H. In some embodiments, R₉ is methyl and R₅, R₆, R₇, or R₈ are H. In some embodiments, R₄ is halogen. In some embodiments, R₄ is —OH, —OR¹⁰, —O—C(═O)—R¹⁰, —C(═O)—OR¹⁰, —O—C(═O)—OR¹⁰—, —C(═O)—O—C(═O)—R¹⁰, —O—C(═O)—C₀₋₉ alkylene-R¹¹, —(O)—C₀₋₉ alkylene-)ₙR¹¹, —O—C(═O)—C₀₋₉ alkylene-N(R¹⁰)₂, —N(R¹⁰)₂, or —C(═O)—C₀₋₉ alkylene-N(R¹⁰)₂.

In some embodiments, the compound of Formula I′ has a structure of Formula I′-a, or an N-oxide, or a pharmaceutically acceptable salt thereof:

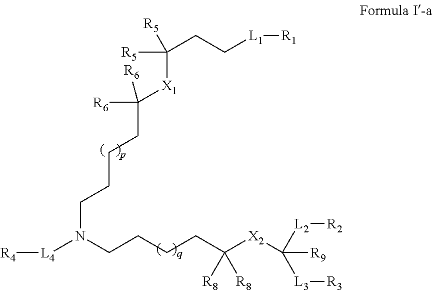

wherein:
L₁ is substituted or unsubstituted linear —C₃₋₁₂ alkylene or —C₃₋₁₂ alkenylalkylene;
L₂ is substituted or unsubstituted linear —C₄₋₁₂ alkylene or —C₃₋₁₂ alkenylalkylene;
L₃ is substituted or unsubstituted linear —C₄₋₁₂ alkylene or —C₃₋₁₂ alkenylalkylene;
wherein if L₁, L₂, or L₃ is substituted, then L₁, L₂, or L₃ is substituted with 1-5 substituents selected from the group consisting of halogen, deuterium, —CN, linear or branched C₁₋₁₀ alkyl, linear or branched C₁₋₁₀ heteroalkyl, C₁₋₂ haloalkyl, —C(═O)R¹⁰, —C(═O)N(R¹⁰)₂, —OR¹⁰, —N(R¹⁰)₂, substituted or unsubstituted C₃₋₆ cycloalkyl, and substituted or unsubstituted 3- to 10-membered heterocycloalkyl; wherein each substituted C₃₋₆ cycloalkyl and substituted 3- to 10-membered heterocycloalkyl is substituted with 1-5 R¹¹;
L₄ is substituted or unsubstituted —C₁₋₂₄ alkylene or —C₃₋₂₄ alkenylalkylene; wherein L₄ is optionally substituted with 1 to 10 R¹¹;
X₁ is a covalent bond, —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—;
X₂ is a covalent bond, —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—;
each R₁, R₂, and R₃, is independently hydrogen, deuterium, halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl;
provided that at least one of R₁, R₂, and R₃ is not hydrogen or deuterium;
or when each R₁, R₂, and R₃ is hydrogen, or deuterium, then at least one of L₁, L₂, L₃ is substituted with 1-5 substituents, and at least one of the substituents is not a linear C₁₋₁₀ alkyl substituted at a terminal carbon, or a deuterium;
R₄ is halogen, —OH, —OR¹⁰, —O—C(═O)—R¹⁰, —C(═O)—OR¹⁰, —O—C(═O)—OR¹⁰—, —C(═O)—O—C(═O)—R¹⁰, —O—C(═O)—C₀₋₉ alkylene-R¹¹, —(O—C₀₋₉ alkylene-)ₙR¹¹, —O—C(═O)—C₀₋₉ alkylene-N(R¹⁰)₂, —N(R¹⁰)₂, or —C(═O)—C₀₋₉ alkylene-N(R¹⁰)₂;
each R₅, R₆, R₈, or R₉ is independently hydrogen, deuterium, halogen, —CN, —OR¹⁰, —N(R¹⁰)₂, —O—C(═O)—R¹⁰, C₁₋₁₀ alkyl, C₁₋₁₀ heteroalkyl, C₃₋₁₀ cycloalkyl, C₃₋₁₀ heterocycloalkyl, C₂₋₆ alkenyl, or C₂₋₆ alkynyl;
each R¹⁰ is independently hydrogen, C₁₋₁₀ alkyl, C₁₋₁₀ heteroalkyl, C₁₋₁₀ haloalkyl, C₃₋₁₀ cycloalkyl, C₃₋₁₀ heterocycloalkyl, C₂₋₆ alkenyl, or C₂₋₆ alkynyl;
each R¹¹ is independently hydrogen, deuterium, halogen, —CN, C₁₋₁₀ alkyl, C₁₋₁₀ heteroalkyl, C₁₋₁₀ haloalkyl, C₃₋₁₀ cycloalkyl, C₃₋₁₀ heterocycloalkyl, C₂₋₆ alkenyl, C₂₋₆ alkynyl, —C(O)OH, —C(O)—O—C₁₋₆ alkyl, —C(O)—C₁₋₆ alkyl, —C(O)NH₂, —C(O)NH(C₁₋₆ alkyl), —C(O)N(C₁₋₆ alkyl)₂, —NH₂, —NH(C₁₋₆ alkyl), —N(C₁₋₆ alkyl)₂, —OH, —O—C₁₋₆ alkyl, —SH, —S(C₁₋₆ alkyl), —S(O)(C₁₋₆ alkyl), —S(O)₂(C₁₋₆ alkyl), or —S(O)₂NH(C₁₋₆ alkyl);
n is an integer from 0-20;
p is 0, 1, 2, 3, 4, or 5; and
q is 0, 1, 2, 3, 4, or 5.

In some embodiments, X₁ is a covalent bond. In some embodiments, X₂ is a covalent bond. In some embodiments, at least one of X₁ and X₂ is not a covalent bond. In some embodiments, X₁ is a covalent bond and X₂ is —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—. In some embodiments, X₂ is a covalent bond and X₁ is —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—. In some embodiments, X₁ is —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—. In some embodiments, X₂ is —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—. In some embodiments, two R₅ are same. In some embodiments, two R₅ are different. In some embodiments, two R₆ are same. In some embodiments, two R₆ are different. In some embodiments, two R₈ are same. In some embodiments, two R₈ are different. In some embodiments, each R₅, R₆, R₈, or R₉ is independently hydrogen, deuterium, halogen, —CN, —OR¹⁰, or C₁₋₁₀ alkyl. In some embodiments, each R₅, R₆, R₈, or R₉ is independently hydrogen, deuterium, halogen, or C₁₋₆ alkyl. In some embodiments, each R₅, R₆, R₈, or R₉ is independently hydrogen, deuterium, fluoro, or methyl. In certain embodiments, each R₅, R₆, R₈, or R₉ is independently hydrogen, deuterium, halogen, methyl, ethyl, or isopropyl. In some embodiments, each R₅, R₆, R₈, or R₉ is independently hydrogen or deuterium. In some embodiments, each R₅, R₆, R₈, or R₉ is independently hydrogen. In certain embodiments, each R₅ is independently hydrogen, halogen or C₁₋₁₀ alkyl, wherein at least one R₅ is not hydrogen, and R₆, R₈, or R₉ are H. In certain embodiments, each R₅ is independently hydrogen, halogen or C₁₋₈ alkyl, wherein at least one R₅ is not hydrogen, and R₆, R₈, or R₉ are H. In certain embodiments, each R₅ is independently hydrogen, halogen or C₁₋₆ alkyl, wherein at least one R₅ is not hydrogen, and R₆, R₈, or R₉ are H. In some embodiments, each R₆ is hydrogen, halogen or C₁₋₆ alkyl, wherein at least one R₆ is not hydrogen, and R₅, R₈, or R₉ are H. In some embodiments, each R₈ is independently hydrogen, halogen or C₁₋₆ alkyl, wherein at least one R₈ is not hydrogen, and R₅, R₆, or R₉ are H. In some embodiments, R₉ is hydrogen, halogen or C₁₋₆ alkyl, and R₅, R₆, or R₈ are H. In some embodiments, R₉ is not hydrogen, and R₅, R₆, or R₈ are H. In some embodiments, R₉ is halogen or C₁₋₆ alkyl, and R₅, R₆, or R₈ are H. In some embodiments, one of R₅ is methyl and the other R₅ and R₆, R₈, or R₉ are H. In some embodiments, one of R₅ is ethyl and the other R₅ and R₆, R₈, or R₉ are H. In some embodiments, one of R₅ is C₃ alkyl and the other R₅ and R₆, R₈, or R₉ are H. In some embodiments, one of R₅ is C₄ alkyl and the other R₅ and R₆, R₈, or R₉ are H. In some embodiments, one of R₅ is C₅ alkyl and the other R₅ and R₆, R₈, or R₉ are H. In some embodiments, one of R₅ is C₆ alkyl and the other R₅ and R₆, R₈, or R₉ are H. In some embodiments, one of R₅ is C₇ alkyl and the other R₅ and R₆, R₈, or R₉ are H. In some embodiments, one of R₅ is C₈ alkyl and the other R₅ and R₆, R₈, or R₉ are H. In some embodiments, one of R₅ is C₉ alkyl and the other R₅ and R₆, R₈, or R₉ are H. In some embodiments, one of R₅ is C₁₀ alkyl and the other R₅ and R₆, R₈, or R₉ are H. In some embodiments, one of R₅ is

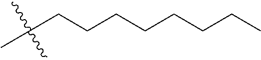 [SMILES: *CCCCCCCC]

and the other R₅ and R₆, R₈, or R₉ are H. In some embodiments, one of R₅ is

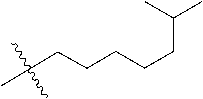 [SMILES: *CCCCCC(C)C]

and the other R₅ and R₆, R₈, or R₉ are H. In some embodiments, one of R₆ is methyl and the other R₆ and R₅, R₈, or R₉ are H. In some embodiments, one of R₈ is methyl and the other R₈ and R₅, R₆, R₇, or R₉ are H. In some embodiments, R₉ is methyl and R₅, R₆, R₇, or R₈ are H. In some embodiments, R₄ is halogen. In some embodiments, R₄ is —OH, —OR¹⁰, —O—C(═O)—R¹⁰, —C(═O)—OR¹⁰, —O—C(═O)—OR¹⁰—, —C(═O)—O—C(═O)—R¹⁰, —O—C(═O)—C₀₋₉ alkylene-R¹¹, —(O—C₀₋₉ alkylene-)ₙR¹¹, —O—C(═O)—C₀₋₉ alkylene-N(R¹⁰)₂, —N(R¹⁰)₂, or —C(═O)—C₀₋₉ alkylene-N(R¹⁰)₂.

Some embodiments described herein include amino lipid compounds having the structure of Formula (I):

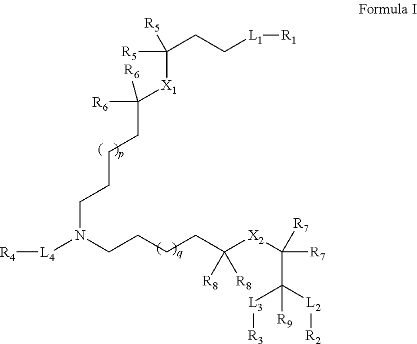

or an N-oxide, or a pharmaceutically acceptable salt thereof,
wherein:
L₁ is substituted or unsubstituted linear —C₃₋₁₂ alkylene or —C₃₋₁₂ alkenylalkylene;
L₂ is substituted or unsubstituted linear —C₄₋₁₂ alkylene or —C₃₋₁₂ alkenylalkylene;
L₃ is substituted or unsubstituted linear —C₄₋₁₂ alkylene or —C₃₋₁₂ alkenylalkylene;
wherein if L₁, L₂, or L₃ is substituted, then L₁, L₂, or L₃ is substituted with 1-5 substituents selected from the group consisting of halogen, deuterium, —CN, linear or branched C₁₋₁₀ alkyl, linear or branched C₁₋₁₀ heteroalkyl, C₁₋₂ haloalkyl, —C(═O)R¹⁰, —C(═O)N(R¹⁰)₂, —OR¹⁰, —N(R¹⁰)₂, substituted or unsubstituted C₃₋₆ cycloalkyl, and substituted or unsubstituted 3- to 10-membered heterocycloalkyl; wherein each substituted C₃₋₆ cycloalkyl and substituted 3- to 10-membered heterocycloalkyl is substituted with 1-5 R¹¹;
L₄ is substituted or unsubstituted —C₁₋₂₄ alkylene or —C₃₋₂₄ alkenylalkylene; wherein L₄ is optionally substituted with 1 to 10 R¹¹;
X₁ is —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—;
X₂ is —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—;
each R₁, R₂, and R₃, is independently hydrogen, deuterium, halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl;
provided that at least one of R₁, R₂, and R₃ is not hydrogen or deuterium;
or when each R₁, R₂, and R₃ is hydrogen, or deuterium, then at least one of L₁, L₂, L₃ is substituted with 1-5 substituents, and at least one of the substituents is not a linear C₁₋₁₀ alkyl substituted at a terminal carbon, or a deuterium;
R₄ is —OH, —OR¹⁰, —O—C(═O)—R¹⁰, —C(═O)—OR¹⁰, —O—C(═O)—OR¹⁰—, —C(═O)—O—C(═O)—R¹⁰, —O—C(═O)—C₀₋₉ alkylene-R¹¹, —(O—C₀₋₉ alkylene-)ₙR¹¹, —O—C(═O)—C₀₋₉ alkylene-N(R¹⁰)₂, —N(R¹⁰)₂, or —C(═O)—C₀₋₉ alkylene-N(R¹⁰)₂;
each R₅, R₆, R₇, R₈, or R₉ is independently hydrogen, deuterium, halogen, —CN, —OR¹⁰, —N(R¹⁰)₂, —O—C(═O)—R¹⁰, C₁₋₁₀ alkyl, C₁₋₁₀ heteroalkyl, C₃₋₁₀ cycloalkyl, C₃₋₁₀ heterocycloalkyl, C₂₋₆ alkenyl, or C₂₋₆ alkynyl;
each R¹⁰ is independently hydrogen, C₁₋₁₀ alkyl, C₁₋₁₀ heteroalkyl, C₁₋₁₀ haloalkyl, C₃₋₁₀ cycloalkyl, C₃₋₁₀ heterocycloalkyl, C₂₋₆ alkenyl, or C₂₋₆ alkynyl;
each R¹¹ is independently hydrogen, deuterium, halogen, —CN, C₁₋₁₀ alkyl, C₁₋₁₀ heteroalkyl, C₁₋₁₀ haloalkyl, C₃₋₁₀ cycloalkyl, C₃₋₁₀ heterocycloalkyl, C₂₋₆ alkenyl, C₂₋₆ alkynyl, —C(O)OH, —C(O)—O—C₁₋₆ alkyl, —C(O)—C₁₋₆ alkyl, —C(O)NH₂, —C(O)NH(C₁₋₆ alkyl), —C(O)N(C₁₋₆ alkyl)₂, —NH₂, —NH(C₁₋₆ alkyl), —N(C₁₋₆ alkyl)₂, —OH, —O—C₁₋₆ alkyl, —SH, —S(C₁₋₆ alkyl), —S(O)(C₁₋₆ alkyl), —S(O)₂(C₁₋₆ alkyl), or —S(O)₂NH(C₁₋₆ alkyl);
n is an integer from 0-20;
p is 0, 1, 2, 3, 4, or 5; and
q is 0, 1, 2, 3, 4, or 5.

In some embodiments, two R₅ are same. In some embodiments, two R₅ are different. In some embodiments, two R₆ are same. In some embodiments, two R₆ are different. In some embodiments, two R₇ are same. In some embodiments, two R₇ are different. In some embodiments, two R₈ are same. In some embodiments, two R₈ are different. In some embodiments, each R₅, R₆, R₇, R₈, or R₉ is independently hydrogen, deuterium, halogen, —CN, —OR¹⁰, or C₁₋₁₀ alkyl. In some embodiments, each R₅, R₆, R₇, R₈, or R₉ is independently hydrogen, deuterium, halogen, or C₁₋₆ alkyl. In some embodiments, each R₅, R₆, R₇, R₈, or R₉ is independently hydrogen, deuterium, fluoro, or methyl. In certain embodiments, each R₅, R₆, R₇, R₈, or R₉ is independently hydrogen, deuterium, halogen, methyl, ethyl, or isopropyl. In certain embodiments, each R₅ is independently hydrogen, halogen or C₁₋₁₀ alkyl, wherein at least one R₅ is not hydrogen, and R₆, R₇, R₈, or R₉ are H. In certain embodiments, each R₅ is independently hydrogen, halogen or C₁₋₈ alkyl, wherein at least one R₅ is not hydrogen, and R₆, R₇, R₈, or R₉ are H. In certain embodiments, each R₅ is independently hydrogen, halogen or C₁₋₆ alkyl, wherein at least one R₅ is not hydrogen, and R₆, R₇, R₈, or R₉ are H. In some embodiments, each R₆ is hydrogen, halogen or C₁₋₆ alkyl, wherein at least one R₆ is not hydrogen, and R₅, R₇, R₈, or R₉ are H. In some embodiments, each R₇ is independently hydrogen, halogen or C₁₋₆ alkyl, wherein at least one R₇ is not hydrogen, and R₅, R₆, R₈, or R₉ are H. In some embodiments, each R₈ is independently hydrogen, halogen or C₁₋₆ alkyl, wherein at least one R₈ is not hydrogen, and R₅, R₆, R₇, or R₉ are H. In some embodiments, R₉ is hydrogen, halogen or C₁₋₆ alkyl, and R₅, R₆, R₇, or R₈ are H. In some embodiments, R₉ is not hydrogen, and R₅, R₆, R₇, or R₈ are H. In some embodiments, R₉ is halogen or C₁₋₆ alkyl, and R₅, R₆, R₇, or R₈ are H. In some embodiments, one of R₅ is methyl and the other R₅ and R₆, R₇, R₈, or R₉ are H. In some embodiments, one of R₅ is ethyl and the other R₅ and R₆, R₇, R₈, or R₉ are H. In some embodiments, one of R₅ is C₃ alkyl and the other R₅ and R₆, R₇, R₈, or R₉ are H. In some embodiments, one of R₅ is C₄ alkyl and the other R₅ and R₆, R₇, R₈, or R₉ are H. In some embodiments, one of R₅ is C₅ alkyl and the other R₅ and R₆, R₇, R₈, or R₉ are H. In some embodiments, one of R₅ is C₆ alkyl and the other R₅ and R₆, R₇, R₈, or R₉ are H. In some embodiments, one of R₅ is C₇ alkyl and the other R₅ and R₆, R₇, R₈, or R₉ are H. In some embodiments, one of R₅ is C₈ alkyl and the other R₅ and R₆, R₇, R₈, or R₉ are H. In some embodiments, one of R₅ is C₉ alkyl and the other R₅ and R₆, R₇, R₈, or R₉ are H. In some embodiments, one of R₅ is C₁₀ alkyl and the other R₅ and R₆, R₇, R₈, or R₉ are H. In some embodiments, one of R₅ is

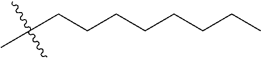 [SMILES: *CCCCCCCC]

and the other R₅ and R₆, R₇, R₈, or R₉ are H. In some embodiments, one of R₅ is

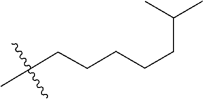 [SMILES: *CCCCCC(C)C]

and the other R₅ and R₆, R₇, R₈, or R₉ are H. In some embodiments, one of R₆ is methyl and the other R₆ and R₅, R₇, R₈, or R₉ are H. In some embodiments, one of R₇ is methyl and the other R₇ and R₅, R₆, R₈, or R₉ are H. In some embodiments, one of R₈ is methyl and the other R₈ and R₅, R₆, R₇, or R₉ are H. In some embodiments, R₉ is methyl and R₅, R₆, R₇, or R₈ are H.

In some embodiments, the compound of Formula I′ or Formula I has a structure of Formula I-a, or an N-oxide, or a pharmaceutically acceptable salt thereof:

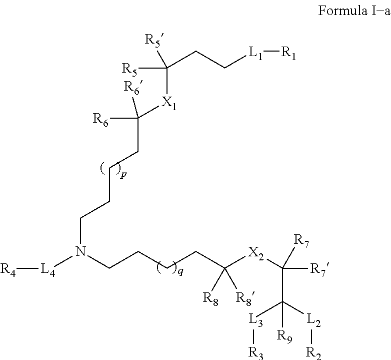

wherein each R₅, R₅′, R₆, R₆′, R₇, R₇′, R₈, R₈′, or R₉ is independently hydrogen, deuterium, halogen, —CN, —OR¹⁰, —N(R¹⁰)₂, —O—C(═O)—R¹⁰, C₁₋₁₀ alkyl, C₁₋₁₀ heteroalkyl, C₃₋₁₀ cycloalkyl, C₃₋₁₀ heterocycloalkyl, C₂₋₆ alkenyl, or C₂₋₆ alkynyl.

In some embodiments, R₅ and R₅′ are same. In some embodiments, R₅ and R₅′ are different. In some embodiments, R₆ and R₆′ are same. In some embodiments, R₆ and R₆′ are different. In some embodiments, R₇ and R₇′ are same. In some embodiments, R₇ and R₇′ are different. In some embodiments, R₈ and R₈′ are same. In some embodiments, R₈ and R₈′ are different. In some embodiments, each R₅, R₅′, R₆, R₆′, R₇, R₇′, R₈, R₈′, or R₉ is independently hydrogen, deuterium, halogen, —CN, —OR¹⁰, or C₁₋₁₀ alkyl. In some embodiments, each R₅, R₅′, R₆, R₆′, R₇, R₇′, R₈, R₈′, or R₉ is independently hydrogen, deuterium, halogen, or C₁₋₆ alkyl. In some embodiments, each R₅, R₅′, R₆, R₆′, R₇, R₇′, R₈, R₈′, or R₉ is independently hydrogen, deuterium, fluoro, or methyl. In certain embodiments, each R₅, R₅′, R₆, R₆′, R₇, R₇′, R₈, R₈′, or R₉ is independently hydrogen, deuterium, halogen, methyl, ethyl, or isopropyl. In certain embodiments, R₅ is halogen or C₁₋₁₀ alkyl, and R₅′, R₆, R₆′, R₇, R₇′, R₈, R₈′, or R₉ are H. In certain embodiments, R₅ is halogen or C₁₋₈ alkyl, and R₅′, R₆, R₆′, R₇, R₇′, R₈, R₈′, or R₉ are H. In certain embodiments, R₅ is halogen or C₁₋₆ alkyl, and R₅′ R₆, R₆′, R₇, R₇′, R₈, R₈′, or R₉ are H. In some embodiments, R₆ is halogen or C₁₋₆ alkyl, and R₅′, R₅, R₆′, R₇, R₇′, R₈, R₈′, or R₉ are H. In some embodiments, R₇ is halogen or C₁₋₆ alkyl, and R₅′, R₅, R₆′, R₆, R₇′, R₈, R₈′, or R₉ are H. In some embodiments, R₈ is halogen or C₁₋₆ alkyl, and R₅′, R₅, R₆′, R₆, R₇′, R₇, R₈′, or R₉ are H. In some embodiments, R₉ is halogen or C₁₋₆ alkyl, and R₅′, R₅, R₆′, R₆, R₇′, R₇, R₈′, or R₉ are H. In some embodiments, R₅ is methyl and R₅′. R₆′, R₆, R₇′, R₇, R₈′, R₈ or R₉ are H. In some embodiments, one of R₅ is ethyl and the other R₅ and R₆, R₇, R₈, or R₉ are H. In some embodiments, one of R₅ is C₃ alkyl and R₅′, R₆′, R₆, R₇′, R₇, R₈′, R₈ or R₉ are H. In some embodiments, one of R₅ is C₄ alkyl and R₅′, R₆′, R₆, R₇′, R₇, R₈′, R₈ or R₉ are H. In some embodiments, one of R₅ is C₅ alkyl and R₅′, R₆′, R₆, R₇′, R₇, R₈′, R₈ or R₉ are H. In some embodiments, one of R₅ is C₆ alkyl and R₅′, R₆′, R₆, R₇′, R₇, R₈′, R₈ or R₉ are H. In some embodiments, one of R₅ is C₇ alkyl and R₅′, R₆′, R₆, R₇′, R₇, R₈′, R₈ or R₉ are H. In some embodiments, one of R₅ is C₈ alkyl and R₅′, R₆′, R₆, R₇′, R₇, R₈′, R₈ or R₉ are H. In some embodiments, one of R₅ is C₉ alkyl and R₅′, R₆′, R₆, R₇′, R₇, R₈′, R₈ or R₉ are H. In some embodiments, one of R₅ is C₁₀ alkyl and R₅′, R₆′, R₆, R₇′, R₇, R₈′, R₈ or R₉ are H. In some embodiments, R₅ is

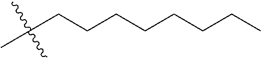 [SMILES: *CCCCCCCC]

and R₅′, R₆′, R₆, R₇′, R₇, R₈′, R₈ or R₉ are H. In some embodiments, R₅ is

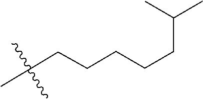 [SMILES: *CCCCCC(C)C]

and R₅′, R₆′, R₆, R₇′, R₇, R₈′, R₈ or R₉ are H. In some embodiments, one of R₆ is methyl and R₅′, R₅, R₆′, R₇′, R₇, R₈′, R₈ or R₉ are H. In some embodiments, one of R₇ is methyl and R₅′, R₅, R₆′, R₆, R₇′, R₈′, R₈ or R₉ are H. In some embodiments, one of R₈ is methyl and R₅′, R₅, R₆′, R₆, R₇′, R₇, R₈′ or R₉ are H. In some embodiments, R₉ is methyl and R₅′, R₅, R₆′, R₆, R₇′, R₇, R₈′ or R₈ are H.

In some embodiments, a compound of Formula I′ or Formula I has the structure of Formula I-b, or an N-oxide, or a pharmaceutically acceptable salt thereof:

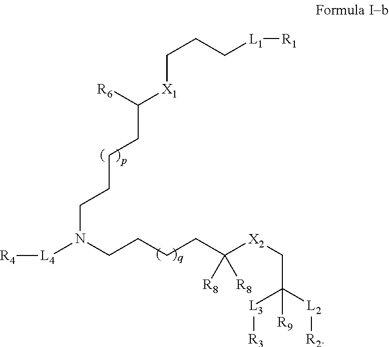

In some embodiments, R₆ is hydrogen, deuterium, halogen, —CN, —OR¹⁰, or C₁₋₁₀ alkyl. In some embodiments, R₆ is hydrogen. In some embodiments, R₆ is methyl.

In some embodiments, a compound of Formula I′-a or Formula I has the structure of Formula II, or an N-oxide, or a pharmaceutically acceptable salt thereof:

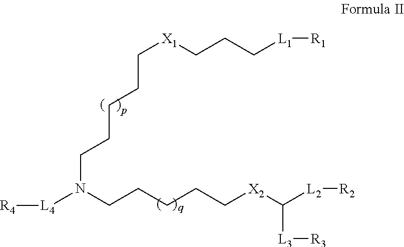

or an N-oxide, or a pharmaceutically acceptable salt thereof, wherein:
L₁ is substituted or unsubstituted linear —C₃₋₁₂ alkylene;
L₂ is substituted or unsubstituted linear —C₄₋₁₂ alkylene;
L₃ is substituted or unsubstituted linear —C₄₋₁₂ alkylene;
wherein if L₁, L₂, or L₃ is substituted, then L₁, L₂, or L₃ is substituted with 1-5 substituents selected from the group consisting of halogen, deuterium, —CN, linear or branched C₁₋₆ alkyl, C₁₋₂ haloalkyl, —C(═O)R¹⁰, —C(═O)N(R¹⁰)₂, —OR¹⁰, —N(R¹⁰)₂, substituted or unsubstituted C₃₋₆ cycloalkyl, and substituted or unsubstituted 3- to 6-membered heterocycloalkyl; wherein each substituted C₃₋₆ cycloalkyl and substituted 3- to 6-membered heterocycloalkyl is substituted with 1-5 R¹¹;
L₄ is substituted or unsubstituted —C₁₋₂₄ alkylene-; wherein La is optionally substituted with 1 to 10 R¹¹;
X₁ is —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—;
X₂ is —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—;
each R₁, R₂, and R₃, is independently hydrogen, deuterium, halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl;
   provided that at least one of R₁, R₂, and R₃ is not hydrogen or deuterium;
      or when each R₁, R₂, and R₃ is hydrogen or deuterium, then at least one of L₁, L₂, L₃ is substituted with 1-5 substituents, and at least one of the substituents is not a linear C₁₋₁₀ alkyl substituted at a terminal carbon, or a deuterium;
R₄ is —OH, —OR¹⁰, —O—C(═O)—R¹⁰, —C(═O)—OR¹⁰, —O—C(═O)—OR¹⁰—, —C(═O)—O—C(═O)—R¹⁰, —O—C(═O)—C₀₋₉ alkylene-R¹¹, —(O—C₀₋₉ alkylene-)ₙR¹¹, —O—C(═O)—C₀₋₉ alkylene-N(R¹⁰)₂, —N(R¹⁰)₂, —CH(CH₃)—CH₂—OH, or —C(═O)—C₀₋₉ alkylene-N(R¹⁰)₂;
each R¹⁰ is independently hydrogen, C₁₋₁₀ alkyl, C₁₋₁₀ haloalkyl, C₁₋₁₀ heteroalkyl, C₃₋₁₀ heterocycloalkyl, C₂₋₆ alkenyl, C₂₋₆ alkynyl or C₃₋₁₀ cycloalkyl;
each R¹¹ is independently selected from the group consisting of hydrogen, deuterium, halogen, —CN, —C₁₋₆ alkyl, C₁₋₁₀ heteroalkyl, C₁₋₁₀ haloalkyl, C₃₋₁₀ heterocycloalkyl, C₂₋₆ alkenyl, C₂₋₆ alkynyl, —C(O)OH, —C(O)—O—C₁₋₆ alkyl, —C(O)—C₁₋₆ alkyl, —C(O)NH₂, —C(O)NH(C₁₋₆ alkyl), —C(O)N(C₁₋₆ alkyl)₂, —NH₂, —NH(C₁₋₆ alkyl), —N(C₁₋₆ alkyl)₂, —OH, —O—C₁₋₆ alkyl, —SH, —S(C₁₋₆ alkyl), —S(O)(C₁₋₆ alkyl), —S(O)₂(C₁₋₆ alkyl), or —S(O)₂NH(C₁₋₆ alkyl);
n is an integer from 0-20;
p is 0, 1, 2, 3, or 4; and
q is 0, 1, 2, 3, or 4.

In some embodiments, a compound of Formula I′-a or Formula I has the structure of Formula II, or an N-oxide, or a pharmaceutically acceptable salt thereof:

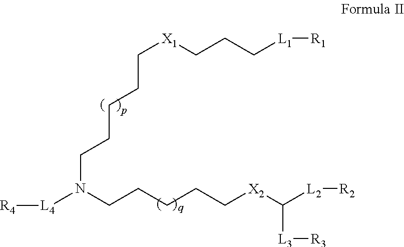

or an N-oxide, or a pharmaceutically acceptable salt thereof, wherein:
L₁ is unsubstituted linear —C₄₋₈ alkylene;
L₂ is unsubstituted linear —C₆₋₁₀ alkylene;
L₃ is unsubstituted linear —C₆₋₁₀ alkylene;
L₄ is unsubstituted —C₁₋₄ alkylene-;
X₁ is —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—;
X₂ is —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—;
each R₁, R₂, and R₃, is independently hydrogen, deuterium, halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl;
   provided that at least one of R₁, R₂, and R₃ is not hydrogen or deuterium;
      or when each R₁, R₂, and R₃ is hydrogen or deuterium, then at least one of L₁, L₂, L₃ is substituted with 1-5 substituents, and at least one of the substituents is not a linear C₁₋₁₀ alkyl substituted at a terminal carbon, or a deuterium;
R₄ is —OH, —OR¹⁰, or —O—C(═O)—C₀₋₉ alkylene-N(R¹⁰)₂;
each R¹⁰ is independently hydrogen or C₁₋₃ alkyl;
p is 0, 1, 2, 3, or 4; and
q is 0, 1, 2, 3, or 4.

In some embodiments, a compound of Formula I′-a or Formula I has the structure of Formula II, or an N-oxide, or a pharmaceutically acceptable salt thereof:

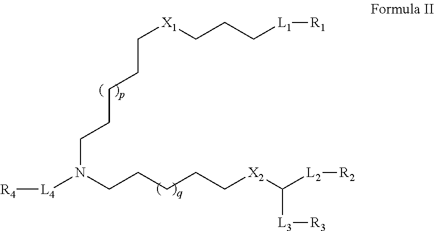

or an N-oxide, or a pharmaceutically acceptable salt thereof, wherein:
L₁ is unsubstituted linear —C₄₋₈ alkylene;
L₂ is unsubstituted linear —C₆₋₁₀ alkylene;
L₃ is unsubstituted linear —C₆₋₁₀ alkylene;
L₄ is unsubstituted —C₁₋₄ alkylene-;
X₁ is —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—;
X₂ is —C(═O)—O—, —O—C(═O)—;
each R₁, R₂, and R₃, is independently hydrogen, deuterium, halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl;
   provided that at least one of R₁, R₂, and R₃ is not hydrogen or deuterium;
R₄ is —OH, or —O—C(═O)—C₃alkylene-N(Me)₂;
each R¹⁰ is independently hydrogen or C₁₋₃ alkyl;
p is 0, 1, 2, 3, or 4; and
q is 0, 1, 2, 3, or 4.

In some embodiments, a compound of Formula I′-a or Formula I has the structure of Formula II, or an N-oxide, or a pharmaceutically acceptable salt thereof:

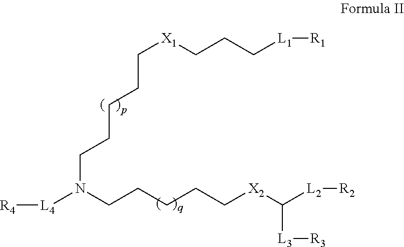

or an N-oxide, or a pharmaceutically acceptable salt thereof, wherein:
L₁ is unsubstituted linear —C₄₋₈ alkylene;
L₂ is unsubstituted linear —C₆₋₁₀ alkylene;
L₃ is unsubstituted linear —C₆₋₁₀ alkylene;
L₄ is unsubstituted —C₁₋₄ alkylene-;
X₁ is —C(═O)—O— or —O—C(═O;
X₂ is —C(═O)—O— or —O—C(═O)—;
each R₁, R₂, and R₃, is independently hydrogen, deuterium, halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl;
   provided that at least one of R₁, R₂, and R₃ is not hydrogen or deuterium;
R₄ is —OH, or —O—C(═O)—C₃alkylene-N(Me)₂;
each R¹⁰ is independently hydrogen or C₁₋₃ alkyl;
p is 0, 1, 2, 3, or 4; and
q is 0, 1, 2, 3, or 4.

In some embodiments, p is equal to q. In some embodiments, p is not equal to q. In some embodiments, p is 1. In some embodiments, p is 2. In some embodiments, p is 3. In some embodiments, q is 1. In some embodiments, q is 2. In some embodiments, q is 3. In some embodiments, q is 4. In some embodiments, p is 1 and q is 1. In some embodiments, p is 1 and q is 3. In some embodiments, p is 3 and q is 1. In some embodiments, p is 3 and q is 3. In some embodiments, p is 3 and q is 4. In some embodiments, p is 4 and q is 3.

In some embodiments, a compound of Formula I′-a or Formula I has the structure of Formula III, or an N-oxide, or a pharmaceutically acceptable salt thereof:

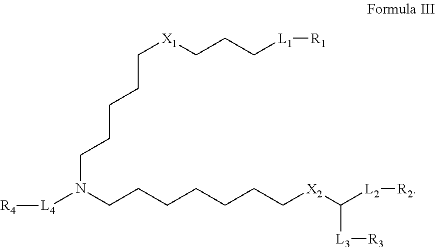

In some embodiments, X; is —C(═O)—O—, —O—C(═O)—, —O—C(═O)—O—, —C(═O)—N(R¹⁰)—, —N(R¹⁰)—C(═O)—, —S—S—, —C(═O)—S—, or —C(C═S)—O—. In some embodiments, X₁ is —C(═O)—O—, —O—C(═O)—, —O—C(═O)—O—, —C(═O)—N(R¹⁰)—, or —N(R¹⁰)—C(═O)—. In some embodiments, X₁ is —C(═O)—O— or —O—C(═O)—. In some embodiments, X₁ is —C(═O)—O—, —O—C(═O)—, —S—S—, —C(═O)—S—, or —C(C═S)—O—. In some embodiments, X₁ is —C(═O)—O—. In some embodiments, X₁ is —O—C(═O)—.

In some embodiments, X₂ is —C(═O)—O—, —O—C(═O)—, —O—C(═O)—O—, —C(═O)—N(R¹⁰)—, —N(R¹⁰)—C(═O)—, —S—S—, —C(═O)—S—, or —C(C═S)—O—. In some embodiments, X₂ is —C(═O)—O—, —O—C(═O)—, —O—C(═O)—O—, —C(═O)—N(R¹⁰)—, or —N(R¹⁰)—C(═O)—. In some embodiments, X₂ is —C(═O)—O— or —O—C(═O)—. In some embodiments, X₂ is —C(═O)—O—, —O—C(═O)—, —S—S—, —C(═O)—S—, or —C(C═S)—O—. In some embodiments, X₂ is —C(═O)—O—. In some embodiments, X₂ is —O—C(═O)—.

In some embodiments, X₁ is —C(═O)—O— and X₂ is —O—C(═O)—. In some embodiments, X₁ is —C(═O)—O— and X₂ is —C(═O)—O—. In some embodiments, X₁ is —O—C(═O)— and X₂ is —O—C(═O)—. In some embodiments, X₁ is —O—C(═O)— and X₂ is —C(═O)—O—.

In some embodiments, a compound of Formula I′-a or Formula I has the structure of Formula III-a, or an N-oxide, or a pharmaceutically acceptable salt thereof:

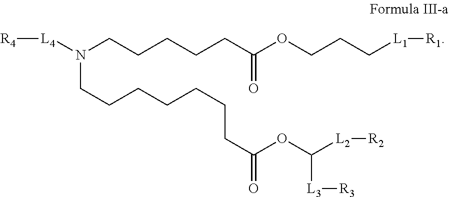

In some embodiments, a compound of Formula I′-a or Formula I has the structure of Formula IV, or an N-oxide, or a pharmaceutically acceptable salt thereof:

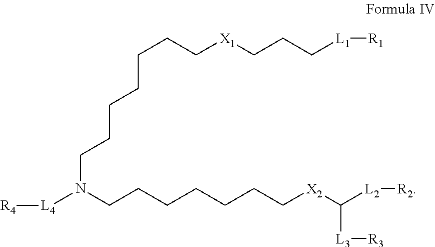

In some embodiments, X₁ is —C(═O)—O—, —O—C(═O)—, —O—C(═O)—O—, —C(═O)—N(R¹⁰)—, —N(R¹⁰)—C(═O)—, —S—S—, —C(═O)—S—, or —C(C═S)—O—. In some embodiments, X₁ is —C(═O)—O—, —O—C(═O)—, —O—C(═O)—O—, —C(═O)—N(R¹⁰)—, or —N(R¹⁰)—C(═O))—. In some embodiments, X₁ is —C(═O)—O— or —O—C(═O)—. In some embodiments, X₁ is —C(═O)—O—, —O—C(═O)—, —S—S—, —C(═O)—S—, or —C(C═S)—O—. In some embodiments, X₁ is —C(═O)—O—. In some embodiments, X₁ is —O—C(═O)—.

In some embodiments, X₂ is —C(═O)—O—, —O—C(═O)—, —O—C(═O)—O—, —C(═O)—N(R¹⁰)—, —N(R¹⁰)—C(═O)—, —S—S—, —C(═O)—S—, or —C(C═S)—O—. In some embodiments, X₂ is —C(═O)—O—, —O—C(═O)—, —O—C(═O)—O—, —C(═O)—N(R¹⁰)—, or —N(R¹⁰)—C(═O)—. In some embodiments, X₂ is —C(═O)—O— or —O—C(═O)—. In some embodiments, X₂ is —C(═O)—O—, —O—C(═O)—, —S—S—, —C(═O)—S—, or —C(C═S)—O—. In some embodiments, X₂ is —C(═O)—O—. In some embodiments, X₂ is —O—C(═O)—.

In some embodiments, X₁ is —C(═O)—O— and X₂ is —O—C(═O)—. In some embodiments, X₁ is —C(═O)—O— and X₂ is —C(═O)—O—. In some embodiments, X₁ is —O—C(═O)— and X₂ is —O—C(═O)—. In some embodiments, X₁ is —O—C(═O)— and X₂ is —C(═O)—O—.

In some embodiments, a compound of Formula I′-a or Formula I has the structure of Formula IV-a, or an N-oxide, or a pharmaceutically acceptable salt thereof:

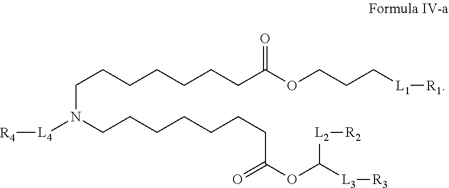

In some embodiments, described herein is a compound of Formula V, or an N-oxide, or a pharmaceutically acceptable salt thereof:

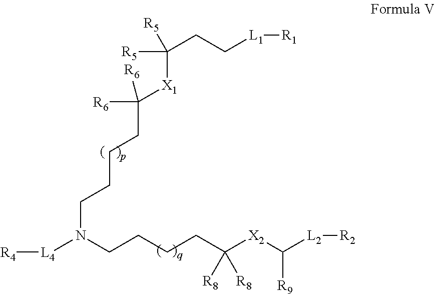

wherein:
L₁ is substituted or unsubstituted linear —C₃₋₁₂ alkylene or —C₃₋₁₂ alkenylalkylene;
L₂ is substituted or unsubstituted linear —C₄₋₁₂ alkylene or —C₃₋₁₂ alkenylalkylene;
wherein if L₁ or L₂ is substituted, then L₁ or L₂ is substituted with 1-5 substituents selected from the group consisting of halogen, deuterium, —CN, linear or branched C₁₋₁₀ alkyl, linear or branched C₁₋₁₀ heteroalkyl, C₁₋₂ haloalkyl, —C(═O)R¹⁰, —C(═O)N(R¹⁰)₂, —OR¹⁰, —N(R¹⁰)₂, substituted or unsubstituted C₃₋₆ cycloalkyl, and substituted or unsubstituted 3- to 10-membered heterocycloalkyl; wherein each substituted C₃₋₆ cycloalkyl and substituted 3- to 10-membered heterocycloalkyl is substituted with 1-5 R¹¹;
L₄ is substituted or unsubstituted —C₁₋₂₄ alkylene or —C₃₋₂₄ alkenylalkylene; wherein L₄ is optionally substituted with 1 to 10 R¹¹;
X₁ is —C(═O)—O—, —O—C(═O)—, —O—C(═O)—O—, —C(═O)—N(R¹⁹)—, —N(R¹⁰)—C(═O)—, S—S, —C(═O)—S—, or —C(C═S)—O—;
X₂ is —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—;
each R₁ and R₂ is independently hydrogen, deuterium, halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl;
R₄ is —OH, —OR¹⁰, —O—C(═O)—R¹⁰, —C(═O)—OR¹⁰, —O—C(═O)—OR¹⁰—, —C(═O)—O—C(═O)—R¹⁰, —O—C(═O)—C₀₋₉ alkylene-R¹¹, —(O—C₀₋₉ alkylene-)ₙR¹¹, —O—C(═O)—C₀₋₉ alkylene-N(R¹⁰)₂, —N(R¹⁰)₂, or —C(═O)—C₀₋₉ alkylene-N(R¹⁰)₂;
each R₅, R₆, R₈, or R₉ is independently hydrogen, deuterium, halogen, —CN, —OR¹⁰, —N(R¹⁰)₂, —O—C(═O)—R¹⁰, C₁₋₁₀ alkyl, C₁₋₁₀ heteroalkyl, C₃₋₁₀ cycloalkyl, C₃₋₁₀ heterocycloalkyl, C₂₋₆ alkenyl, or C₂₋₆ alkynyl;
each R¹⁰ is independently hydrogen, C₁₋₁₀ alkyl, C₁₋₁₀ heteroalkyl, C₁₋₁₀ haloalkyl, C₃₋₁₀ each R¹¹ is independently hydrogen, deuterium, halogen, —CN, C₁₋₁₀ alkyl, C₁₋₁₀ heteroalkyl, C₁₋₁₀ haloalkyl, C₃₋₁₀ cycloalkyl, C₃₋₁₀ heterocycloalkyl, C₂₋₆ alkenyl, C₂₋₆ alkynyl, —C(O)OH, —C(O)—O—C₁₋₆ alkyl, —C(O)—C₁₋₆ alkyl, —C(O)NH₂, —C(O)NH(C₁₋₆ alkyl), —C(O)N(C₁₋₆ alkyl)₂, —NH₂, —NH(C₁₋₆ alkyl), —N(C₁₋₆ alkyl)₂, —OH, —O—C₁₋₆ alkyl, —SH, —S(C₁₋₆ alkyl), —S(O)(C₁₋₆ alkyl), —S(O)₂(C₁₋₆ alkyl), or —S(O)₂NH(C₁₋₆ alkyl);
n is an integer from 0-20;
p is 0, 1, 2, 3, 4, or 5; and
q is 0, 1, 2, 3, 4, or 5.

In some embodiments, described herein is a compound of Formula V, or an N-oxide, or a pharmaceutically acceptable salt thereof:

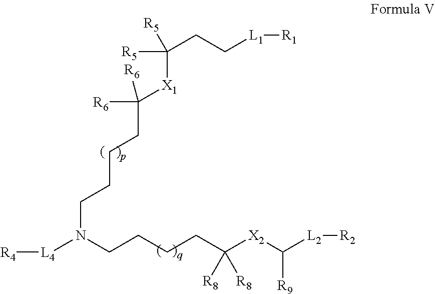

wherein:
L₁ is substituted or unsubstituted linear —C₃₋₁₂ alkylene or —C₃₋₁₂ alkenylalkylene;
L₂ is substituted or unsubstituted linear —C₄₋₁₂ alkylene or —C₃₋₁₂ alkenylalkylene;
wherein if L₁ or L₂ is substituted, then L₁ or L₂ is substituted with 1-5 substituents selected from the group consisting of halogen, deuterium, —CN, linear or branched C₁₋₁₀ alkyl, linear or branched C₁₋₁₀ heteroalkyl, C₁₋₂ haloalkyl, —C(═O)R¹⁰, —C(═O)N(R¹⁰)₂, —OR¹⁰, —N(R¹⁰)₂, substituted or unsubstituted C₃₋₆ cycloalkyl, and substituted or unsubstituted 3- to 10-membered heterocycloalkyl; wherein each substituted C₃₋₆ cycloalkyl and substituted 3- to 10-membered heterocycloalkyl is substituted with 1-5 R₁;
L₄ is substituted or unsubstituted —C₁₋₂₄ alkylene or —C₃₋₂₄ alkenylalkylene; wherein L₄ is optionally substituted with 1 to 10 R₁;
X₁ is —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—;
X₂ is —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—;
each R₁ and R₂ is independently hydrogen, deuterium, halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl;
provided that at least one of R₁ and R₂ is not hydrogen or deuterium;
or when each R₁ and R₂ is hydrogen, or deuterium, then at least one of L₁ and L₂ is substituted with 1-5 substituents, and at least one of the substituents is not a linear C₁₋₁₀ alkyl substituted at a terminal carbon, or a deuterium;
R₄ is —OH, —OR¹⁰, —O—C(═O)—R¹⁰, —C(═O)—OR¹⁰, —O—C(═O)—OR¹⁰—, —C(═O)—O—C(═O)—R¹⁰, —O—C(═O)—C₀₋₉ alkylene-R¹¹, —(O—C₀₋₉ alkylene-)ₙR¹¹, —O—C(═O)—C₀₋₉ alkylene-N(R¹⁰)₂, —N(R¹⁰)₂, or —C(═O)—C₀₋₉ alkylene-N(R¹⁰)₂;
each R₅, R₆, R₈, or R₉ is independently hydrogen, deuterium, halogen, —CN, —OR¹⁰, —N(R¹⁰)₂, —O—C(═O)—R¹⁰, C₁₋₁₀ alkyl, C₁₋₁₀ heteroalkyl, C₃₋₁₀ cycloalkyl, C₃₋₁₀ heterocycloalkyl, C₂₋₆ alkenyl, or C₂₋₆ alkynyl;
each R¹⁰ is independently hydrogen, C₁₋₁₀ alkyl, C₁₋₁₀ heteroalkyl, C₁₋₁₀ haloalkyl, C₃₋₁₀ cycloalkyl, C₃₋₁₀ heterocycloalkyl, C₂₋₆ alkenyl, or C₂₋₆ alkynyl;
each R¹¹ is independently hydrogen, deuterium, halogen, —CN, C₁₋₁₀ alkyl, C₁₋₁₀ heteroalkyl, C₁₋₁₀ haloalkyl, C₃₋₁₀ cycloalkyl, C₃₋₁₀ heterocycloalkyl, C₂₋₆ alkenyl, C₂₋₆ alkynyl, —C(O)OH, —C(O)—O—C₁₋₆ alkyl, —C(O)—C₁₋₆ alkyl, —C(O)NH₂, —C(O)NH(C₁₋₆ alkyl), —C(O)N(C₁₋₆ alkyl)₂, —NH₂, —NH(C₁₋₆ alkyl), —N(C₁₋₆ alkyl)₂, —OH, —O—C₁₋₆ alkyl, —SH, —S(C₁₋₆ alkyl), —S(O)(C₁₋₆ alkyl), —S(O)₂(C₁₋₆ alkyl), or —S(O)₂NH(C₁₋₆ alkyl);
n is an integer from 0-20;
p is 0, 1, 2, 3, 4, or 5; and
q is 0, 1, 2, 3, 4, or 5.

In some embodiments, X₁ is —C(═O)—O—, —O—C(═O)—, —O—C(═O)—O—, —C(═O)—N(R¹⁰)—, —N(R¹⁰)—C(═O)—, —S—S—, —C(═O)—S—, or —C(C═S)—O—. In some embodiments, X₁ is —C(═O)—O—, —O—C(═O)—, —O—C(═O)—O—, —C(═O)—N(R¹⁰)—, or —N(R¹⁰)—C(═O)—. In some embodiments, X₁ is —C(═O)—O— or —O—C(═O)—. In some embodiments, X₁ is —C(═O)—O—, —O—C(═O)—, —S—S—, —C(═O)—S—, or —C(C═S)—O—. In some embodiments, X₁ is —C(═O)—O—. In some embodiments, X₁ is —O—C(═O)—.

In some embodiments, X₂ is —C(═O)—O—, —O—C(═O)—, —O—C(═O)—O—, —C(═O)—N(R¹⁰)—, —N(R¹⁰)—C(═O)—, —S—S—, —C(═O)—S—, or —C(C═S)—O—. In some embodiments, X₂ is —C(═O)—O—, —O—C(═O)—, —O—C(═O)—O—, —C(═O)—N(R¹⁰)—, or —N(R¹⁰)—C(═O)—. In some embodiments, X₂ is —C(═O)—O— or —O—C(═O)—. In some embodiments, X₂ is —C(═O)—O—, —O—C(═O)—, —S—S—, —C(═O)—S—, or —C(C═S)—O—. In some embodiments, X₂ is —C(═O)—O—. In some embodiments, X₂ is —O—C(═O)—.

In some embodiments, X₁ is —C(═O)—O— and X₂ is —O—C(═O)—. In some embodiments, X₁ is —C(═O)—O— and X₂ is —C(═O)—O—. In some embodiments, X₁ is —O—C(═O)— and X₂ is —O—C(═O)—. In some embodiments, X₁ is —O—C(═O)— and X₂ is —C(═O)—O—.

In some embodiments, at least one of R₁, R₂, and R₃ is not hydrogen or deuterium. In some embodiments, R₁, R₂, and R₃ are each independently halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl. In some embodiments, at least one of R₁, R₂, and R₃ is halogen, C₂₋₆ alkenyl, or branched C₃₋₁₀ alkyl. In some embodiments, at least one of R₁, R₂, and R₃ is fluoro or isopropyl. In some aspects, R₁ is halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl. In one aspect, R₁ is halogen, —CN, —OR¹⁰, —N(R¹⁰)₂, C₂₋₆ alkenyl, or branched C₃₋₁₀ alkyl. In another aspect, R₁ is halogen, —OR¹⁰, C₂₋₆ alkenyl, or branched C₃₋₁₀ alkyl. In yet another aspect, R₁ is halogen, —OH, isopropyl, isobutyl, isopentyl, sec-butyl, tert-butyl, tert-pentyl, or tert-hexyl. In some embodiments, R₁ is halogen or isopropyl, isobutyl, sec-butyl, or tert-butyl. In some embodiments, R₁ is halogen or isopropyl. In some embodiments, R₁ is F. In some embodiments. R₁ is Cl. In some embodiments, R₁ is Br. In some embodiments, R₁ is I. In some embodiments, R₁ is isopropyl. In some embodiments, R₁ is C₂₋₆ alkenyl. In some embodiments, R₁ is

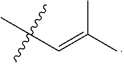 [SMILES: *C=C(C)C]

In some aspects, R₂ is halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl. In one aspect, R₂ is halogen, —CN, —OR¹⁰, —N(R¹⁰)₂, C₂₋₆ alkenyl, or branched C₃₋₁₀ alkyl. In another aspect, R₂ is halogen, —OR¹⁰, C₂₋₆ alkenyl, or branched C₃₋₁₀ alkyl. In yet another aspect, R₂ is halogen, —OH, isopropyl, isobutyl, isopentyl, sec-butyl, tert-butyl, tert-pentyl, or tert-hexyl. In some embodiments, R₂ is halogen or isopropyl, isobutyl, sec-butyl, or tert-butyl. In some embodiments, R₂ is halogen or isopropyl. In some embodiments, R₂ is F. In some embodiments, R₂ is Cl. In some embodiments, R₂ is Br. In some embodiments, R₂ is I. In some embodiments, R₂ is isopropyl. In some aspects, R₃ is halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl. In one aspect, R₃ is halogen, —CN, —OR¹⁰, —N(R¹⁰)₂, C₂₋₆ alkenyl, or branched C₃₋₁₀ alkyl. In another aspect, R₃ is halogen, —OR¹⁰, C₂₋₆ alkenyl, or branched C₃₋₁₀ alkyl. In another aspect, R₃ is halogen, —OH, isopropyl, isobutyl, isopentyl, sec-butyl, tert-butyl, tert-pentyl, or tert-hexyl. In some embodiments, R₃ is halogen or isopropyl, isobutyl, sec-butyl, or tert-butyl. In some embodiments, R₃ is halogen or isopropyl. In some embodiments, R₃ is F. In some embodiments, R₃ is Cl. In some embodiments, R₃ is Br. In some embodiments, R₃ is I. In some embodiments. R₃ is isopropyl. In some embodiments, at least one of R₂ and R₃ is halogen, —CN, —OR¹⁰, —N(R¹⁰)₂, C₂₋₆ alkenyl, or branched C₃₋₁₀ alkyl. In some aspects, R₃ is halogen, —OR¹⁰, or branched C₃₋₁₀ alkyl; and R₁ and R₂ are hydrogen. In another aspect, R₃ is halogen, —OR¹⁰, or branched C₃₋₁₀ alkyl; R₂ is halogen, —OR¹⁰, or branched C₃₋₁₀ alkyl and R₁ is hydrogen. In another aspect, R₃ is halogen, —OR¹⁰, or branched C₃₋₁₀ alkyl; R₂ is halogen, —OR¹⁰, or branched C₃₋₁₀ alkyl and R₁ is C₂₋₆ alkenyl. In some aspects, R₃ is F or branched C₃₋₁₀ alkyl; and R₁ and R₂ are hydrogen. In another aspect, R₃ is F or branched C₃₋₁₀ alkyl; R₂ is F or branched C₃₋₁₀ alkyl and R₁ is hydrogen. In another aspect, R₃ is F or branched C₃₋₁₀ alkyl; R₂ is F or branched C₃₋₁₀ alkyl and R₁ is

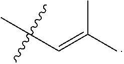 [SMILES: *C=C(C)C]

In some aspects, R₃ is Cl or branched C₃₋₁₀ alkyl; and R₁ and R₂ are hydrogen. In another aspect, R₃ is Cl or branched C₃₋₁₀ alkyl; R₂ is Cl or branched C₃₋₁₀ alkyl and R₁ is hydrogen. In another aspect, R₃ is Cl or branched C₃₋₁₀ alkyl; R₂ is F or branched C₃₋₁₀ alkyl and R₁ is

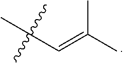 [SMILES: *C=C(C)C]

In some embodiments, R₃ is halogen, —OR¹⁰, C₂₋₆ alkenyl, or branched C₃₋₁₀ alkyl; and R₁ and R₂ are hydrogen. In some embodiments, R₃ is halogen, —OR¹⁰, C₂₋₆ alkenyl, or branched C₃₋₁₀ alkyl; R₂ is halogen, —OR¹⁰, C₂₋₆ alkenyl, or branched C₃₋₁₀ alkyl and R₁ is hydrogen. In some embodiments, R₁ is H; and R₂, and R₃ are each independently halogen, —CN, —OR¹⁰, —N(R¹⁰C)₂, branched C₃₋₁₀ alkyl, C₃₋₁₀ cycloalkyl, C₂₋₆ alkenyl, or C₂₋₆ alkynyl. In some embodiments, R₁ is H; and R₂, and R₃ are each independently halogen, —OR¹⁰, C₂₋₆ alkenyl, or branched C₃₋₆ alkyl. In some embodiments, R₁ is H; R₂ is H, halogen, isopropyl, isobutyl, sec-butyl, or tert-butyl; and R₃ is halogen, —OR¹⁰, or isopropyl, isobutyl, sec-butyl, or tert-butyl. In some embodiments, R₁ is halogen, —OR¹⁰, C₂₋₆ alkenyl, or branched C₃₋₆ alkyl; and R₂, and R₃ are each independently H.

In some embodiments, each R₁, R₂, and R₃ is hydrogen or deuterium, and at least one of L₁, L₂, L₃ is substituted with 1-5 substituents, and at least one of the substituents is not a linear C₁₋₁₀ alkyl substituted at a terminal carbon of L₁, L₂, L₃, or a deuterium. In some embodiments, each R₁, R₂, and R₃ is hydrogen or deuterium, and at least one of L₁, L₂, L₃ is substituted with 1-2 substituents, and at least one of the substituents is not a linear C₁₋₁₀ alkyl substituted at a terminal carbon of L₁, L₂, L₃, or a deuterium. In some embodiments, each R₁, R₂, and R₃ is hydrogen or deuterium, and at least one of L₁, L₂, L₃ is substituted with halogen or linear or branched C₁₋₃ alkyl.

In some embodiments, L₁ is substituted or unsubstituted —C₄₋₁₀ alkylene. In some embodiments, L₁ is substituted or unsubstituted —C₃₋₁₂ alkenylalkylene. In some embodiments, L₁ is —C₄₋₁₀ alkylene substituted with halogen, linear or branched C₁₋₆ alkyl, or C₁₋₂ haloalkyl. In some embodiments, L₁ is —C₄₋₁₀ alkenylalkylene substituted with halogen, linear or branched C₁-6 alkyl, or C₁₋₂ haloalkyl. In some embodiments, L₁ is —C₄₋₁₀ alkylene substituted with fluoro, methyl, or isopropyl.

In some embodiments, L₁ is substituted or unsubstituted linear C₃ alkylene. In some embodiments. L₁ is substituted or unsubstituted linear C₄ alkylene. In some embodiments, L₁ is substituted or unsubstituted linear C₅ alkylene. In some embodiments, L₁ is substituted or unsubstituted linear C₆ alkylene. In some embodiments, L₁ is substituted or unsubstituted linear C₇ alkylene. In some embodiments, L₁ is substituted or unsubstituted linear C₈ alkylene. In some embodiments, L₁ is substituted or unsubstituted linear C₉ alkylene. In some embodiments, L₁ is substituted or unsubstituted linear C₁₀ alkylene. In some embodiments, L₁ is substituted or unsubstituted linear C₁₁ alkylene. In some embodiments, L₁ is substituted or unsubstituted linear C₁₂ alkylene. In some embodiments, L₁ is

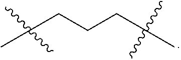 [SMILES: *CCC*]

In some embodiments, L₁ is

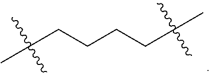 [SMILES: *CCCC*]

In some embodiments, L₁ is

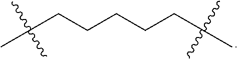 [SMILES: *CCCCC*]

In some embodiments, L₁ is

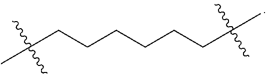 [SMILES: *CCCCCC*]

In some embodiments, L₁ is

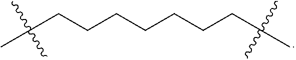 [SMILES: *CCCCCCC*]

In some embodiments, L₁ is

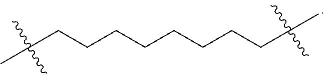 [SMILES: *CCCCCCCC*]

In some embodiments, -L₁-R₁ is

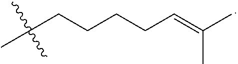 [SMILES: *CCCCC=C(C)C]

In some embodiments, -L₁-R₁ is

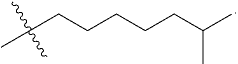 [SMILES: *CCCCCC(C)C]

In some embodiments, -L₁-R₁ is

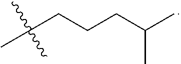 [SMILES: *CCCC(C)C]

In some embodiments, -L₁-R₁ is

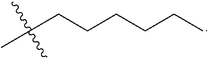 [SMILES: *CCCCCC]

In some embodiments, -L₁-R₁ is

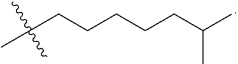 [SMILES: *CCCCCC(C)C]

In some embodiments, -L₁-R₁ is

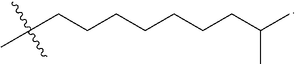 [SMILES: *CCCCCCCC(C)C]

In some embodiments, -L₁-R₁ is

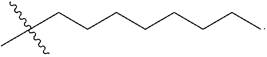 [SMILES: *CCCCCCCC]

In some embodiments, -L₁-R₁ is

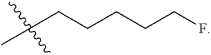 [SMILES: *CCCCCF]

In some embodiments, L₁ is substituted or unsubstituted linear C₃ alkenylalkylene. In some embodiments, L₁ is substituted or unsubstituted linear C₄ alkenylalkylene. In some embodiments, L₁ is substituted or unsubstituted linear C₅ alkenylalkylene. In some embodiments, L₁ is substituted or unsubstituted linear C₆ alkenylalkylene. In some embodiments, L₁ is substituted or unsubstituted linear C₇ alkenylalkylene. In some embodiments, L₁ is substituted or unsubstituted linear C₈ alkenylalkylene. In some embodiments, L₁ is substituted or unsubstituted linear C₉ alkenylalkylene. In some embodiments, L₁ is substituted or unsubstituted linear C₁₀ alkenylalkylene. In some embodiments, L₁ is substituted or unsubstituted linear C₁₁ alkenylalkylene. In some embodiments, L₁ is substituted or unsubstituted linear C₁₂ alkenylalkylene.

In some embodiments, L₂ is substituted or unsubstituted linear C₃ alkylene. In some embodiments, L₂ is substituted or unsubstituted linear C₄ alkylene. In some embodiments, L₂ is substituted or unsubstituted linear C₅ alkylene. In some embodiments, L₂ is substituted or unsubstituted linear C₆ alkylene. In some embodiments, L₂ is substituted or unsubstituted linear C₇ alkylene. In some embodiments, L₂ is substituted or unsubstituted linear C₈ alkylene. In some embodiments, L₂ is substituted or unsubstituted linear C₉ alkylene. In some embodiments, L₂ is substituted or unsubstituted linear C₁₀ alkylene. In some embodiments, L₂ is substituted or unsubstituted linear C₁₁ alkylene. In some embodiments, L₂ is substituted or unsubstituted linear C₁₂ alkylene. In some embodiments, L₂ is

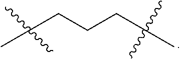 [SMILES: *CCC*]

In some embodiments, L₂ is

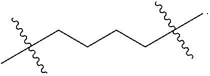 [SMILES: *CCCC*]

In some embodiments, L₂ is

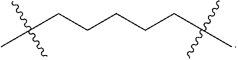 [SMILES: *CCCCC*]

In some embodiments, L₂ is

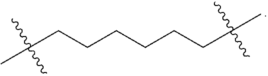 [SMILES: *CCCCCC*]

In some embodiments, L₂ is

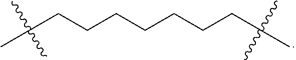 [SMILES: *CCCCCCC*]

In some embodiments, L₂ is

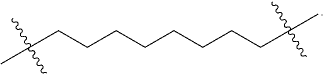 [SMILES: *CCCCCCCC*]

In some embodiments, L₂ is

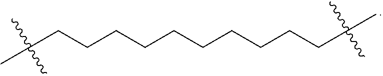 [SMILES: *CCCCCCCCCC*]

In some embodiments, L₂ is substituted or unsubstituted linear C₃ alkenylalkylene. In some embodiments, L₂ is substituted or unsubstituted linear C₄ alkenylalkylene. In some embodiments, L₂ is substituted or unsubstituted linear C₅ alkenylalkylene. In some embodiments. L₂ is substituted or unsubstituted linear C₆ alkenylalkylene. In some embodiments, L₂ is substituted or unsubstituted linear C₇ alkenylalkylene. In some embodiments, L₂ is substituted or unsubstituted linear C₈ alkenylalkylene. In some embodiments, L₂ is substituted or unsubstituted linear C₉ alkenylalkylene. In some embodiments, L₂ is substituted or unsubstituted linear C₁₀ alkenylalkylene. In some embodiments, L₂ is substituted or unsubstituted linear C₁₁ alkenylalkylene. In some embodiments, L₂ is substituted or unsubstituted linear C₁₂ alkenylalkylene.

In some embodiments, -L₂-R₂ is

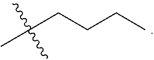 [SMILES: *CCCC]

In some embodiments, -L₂-R₂ is

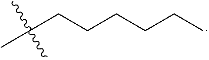 [SMILES: *CCCCCC]

In some embodiments, -L₂-R₂ is

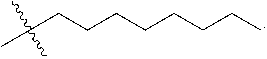 [SMILES: *CCCCCCCC]

In some embodiments, -L₂-R₂ is

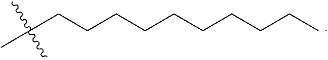 [SMILES: *CCCCCCCCCC]

In some embodiments, -L₂-R₂ is

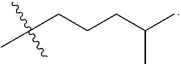 [SMILES: *CCCC(C)C]

In some embodiments, -L₂-R₂ is

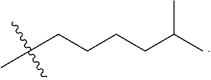 [SMILES: *CCCCC(C)C]

In some embodiments, -L₂-R₂ is

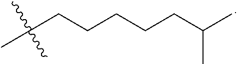 [SMILES: *CCCCCC(C)C]

In some embodiments, -L₂-R₂ is

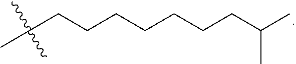 [SMILES: *CCCCCCCC(C)C]

In some embodiments, -L₂-R₂ is

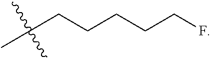 [SMILES: *CCCCCF]

In some embodiments, -L₂-R₂ is

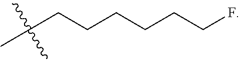 [SMILES: *CCCCCCF]

In some embodiments, -L₂-R₂ is

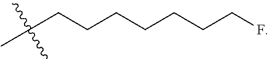 [SMILES: *CCCCCCCF]

In some embodiments, -L₂-R₂ is

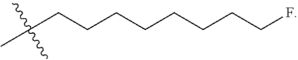 [SMILES: *CCCCCCCCF]

In some embodiments, -L₂-R₂ is

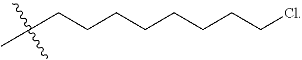 [SMILES: *CCCCCCCCCl]

In some embodiments, -L₂-R₂ is

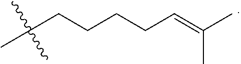 [SMILES: *CCCCC=C(C)C]

In some embodiments, L₃ is substituted or unsubstituted linear C₃ alkylene. In some embodiments. L₃ is substituted or unsubstituted linear C₄ alkylene. In some embodiments, L₃ is substituted or unsubstituted linear C₅ alkylene. In some embodiments, L₃ is substituted or unsubstituted linear C₆ alkylene. In some embodiments, L₃ is substituted or unsubstituted linear C₇ alkylene. In some embodiments, L₃ is substituted or unsubstituted linear C₈ alkylene. In some embodiments, L₃ is substituted or unsubstituted linear C₉ alkylene. In some embodiments, L₃ is substituted or unsubstituted linear C₁₀ alkylene. In some embodiments, L₃ is substituted or unsubstituted linear C₁₁ alkylene. In some embodiments, L₃ is substituted or unsubstituted linear C₁₂ alkylene. In some embodiments, L₃ is

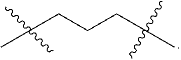 [SMILES: *CCC*]

In some embodiments, L₃ is

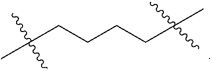 [SMILES: *CCCC*]

In some embodiments, L₃ is

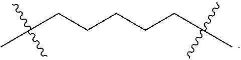 [SMILES: *CCCCC*]

In some embodiments, L₃ is

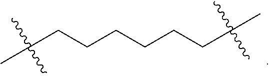 [SMILES: *CCCCCC*]

In some embodiments, L₃ is

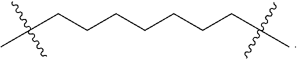 [SMILES: *CCCCCCC*]

In some embodiments, L₃ is

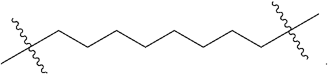 [SMILES: *CCCCCCCC*]

In some embodiments, L₃ is

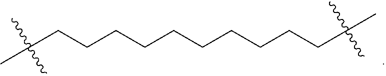 [SMILES: *CCCCCCCCCC*]

In some embodiments, L₃ is substituted or unsubstituted linear C₃ alkenylalkylene. In some embodiments, L₃ is substituted or unsubstituted linear C₄ alkenylalkylene. In some embodiments, L₃ is substituted or unsubstituted linear C₅ alkenylalkylene. In some embodiments, L₃ is substituted or unsubstituted linear C₆ alkenylalkylene. In some embodiments, L₃ is substituted or unsubstituted linear C₇ alkenylalkylene. In some embodiments, L₃ is substituted or unsubstituted linear C₈ alkenylalkylene. In some embodiments, L₃ is substituted or unsubstituted linear C₉ alkenylalkylene. In some embodiments, L₃ is substituted or unsubstituted linear C₁₀ alkenylalkylene. In some embodiments, L₃ is substituted or unsubstituted linear C₁₁ alkenylalkylene. In some embodiments. L₃ is substituted or unsubstituted linear C₁₂ alkenylalkylene.

In some embodiments, -L₃-R₃ is

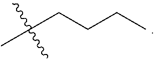 [SMILES: *CCCC]

In some embodiments, -L₃-R₃ is

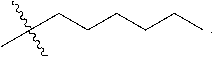 [SMILES: *CCCCCC]

In some embodiments, -L₃-R₃ is

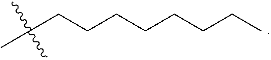 [SMILES: *CCCCCCCC]

In some embodiments, -L₃-R₃ is

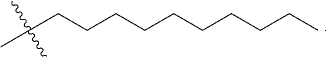 [SMILES: *CCCCCCCCCC]

In some embodiments, -L₃-R₃ is

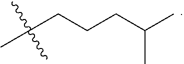 [SMILES: *CCCC(C)C]

In some embodiments, -L₃-R₃ is

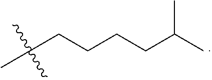 [SMILES: *CCCCC(C)C]

In some embodiments, -L₃-R₃ is

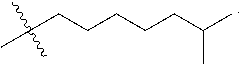 [SMILES: *CCCCCC(C)C]

In some embodiments, -L₃-R₃ is

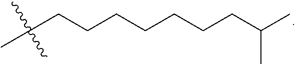 [SMILES: *CCCCCCCC(C)C]

In some embodiments, -L₃-R₃ is

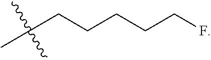 [SMILES: *CCCCCF]

In some embodiments, -L₃-R₃ is

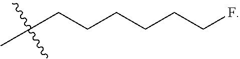 [SMILES: *CCCCCCF]

In some embodiments, -L₃-R₃ is

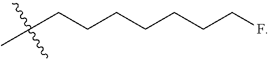 [SMILES: *CCCCCCCF]

In some embodiments, -L₃-R₃ is

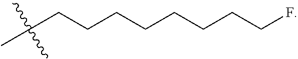 [SMILES: *CCCCCCCCF]

In some embodiments, -L₃-R₃ is

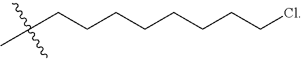 [SMILES: *CCCCCCCCCl]

In some embodiments, -L₃-R₃ is

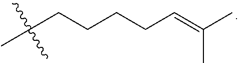 [SMILES: *CCCCC=C(C)C]

In some embodiments, each L₂ and L₃ is independently substituted or unsubstituted —C₄₋₁₀ alkylene. In some embodiments, each L₂ and L₃ is independently substituted or unsubstituted —C₃₋₁₂ alkenylalkylene. In some embodiments, L₂ and L₃ are the same. In some embodiments, L₂ and L₃ are different. In some embodiments, each L₂ and L₃ is independently substituted —C₄₋₁₀ alkylene, wherein the substituent is halogen, or linear or branched C₁₋₆ alkyl. In some embodiments, each L₂ and L₃ is independently a substituted —C₄₋₁₀ alkylene, wherein —C₄₋₁₀ alkylene is substituted with fluoro, methyl, or isopropyl. In some embodiments, each L₂ and L₃ is independently substituted —C₄₋₁₀ alkenylalkylene, wherein the substituent is halogen, or linear or branched C₁₋₆ alkyl. In some embodiments, each L₂ and L₃ is independently a substituted —C₄₋₁₀ alkenylalkylene, wherein —C₄₋₁₀ alkylene is substituted with fluoro, methyl, or isopropyl. In some embodiments, each L₂ and L₃ is independently unsubstituted —C₂₋₆ alkylene. In some embodiments, each L₂ and L₃ is independently unsubstituted —C₂₋₄ alkylene. In some embodiments, each L₂ and L₃ is independently unsubstituted —C₃₋₆ alkylene. In some embodiments, L₂ is substituted or unsubstituted —C₃₋₁₂ alkenylalkylene. In some embodiments, L₃ is substituted or unsubstituted —C₃₋₁₂ alkenylalkylene. In some embodiments, each L₂ is independently unsubstituted —C₃₋₆ alkylene and L₃ is unsubstituted —C₃₋₆ alkenylalkylene.

In some embodiments, L₄ is substituted or unsubstituted —C₁₋₁₂ alkylene; wherein L₄ is optionally substituted with 1 to 10 R¹¹. In some embodiments, L₄ is substituted or unsubstituted —C₃₋₂₄ alkenylalkylene, wherein L₄ is optionally substituted with 1 to 10 R¹¹. In some embodiments, L₄ is substituted or unsubstituted —C₃₋₁₂ alkenylalkylene. In some embodiments, L₄ is substituted or unsubstituted —C₃₋₆ alkenylalkylene. In some embodiments, L₄ is substituted or unsubstituted —C₁₋₆ alkylene; wherein L₄ is optionally substituted with 1 to 10 R¹¹. In some embodiments, L₄ is —CH₂—, —CH₂—CH₂—, —CH₂—CH₂—CH₂—, —CH₂—(CH₂)₂—CH₂—, —CH₂—(CH₂)₃—CH₂—, or —CH₂—(CH₂)₄—CH₂—. In some embodiments, L₄ is —CH₂—, —CH₂—CH₂—, or —CH₂—CH₂—CH₂—. In some embodiments, L₄ is —CH₂—. In some embodiments, L₄ is —CH₂—CH₂—. In some embodiments, L₄ is —CH₂—CH₂—CH₂—. In some embodiments, L₄ is —CH₂—CH(CH₃)—CH₂—. In some embodiments, L₄ is —CD₂—CD₂—.

In some embodiments, R₄ is —OH, —OR¹⁰, —O—C(═O)—R¹⁰, —C(═O)—OR¹⁰, —O—C(═O)—C₀₋₉ alkylene-N(R¹¹)₂, —O—C(═O)—C₀₋₉ alkylene-R¹¹, —(O—C₀₋₉ alkylene-)ₙR¹¹, —CH(CH₃)—CH₂—OH, or —C(═O)—C₀₋₉ alkylene-N(R¹¹)₂. In some embodiments, R₄ is —OH, —CH(CH₃)—CH₂—OH, or —O—C(═O)—(CH₂)₃—N(Me)₂. In some embodiments, R₄ is —OH or —O—C(═O)—C₂₋₄ alkylene-N(R¹¹)₂. In some embodiments, R₄ is —OH or —O—C(═O)—C₃₋₆ alkylene-N(R¹¹)₂. In some embodiments, R₄ is —OH. In some embodiments, R₄ is —O—C(═O)—C₃₋₆ alkylene-N(Me)₂. In some embodiments, R₄ is halogen. In some embodiments, R₄ is F.

In some embodiments, -L₄-R₄ is

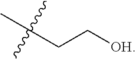 [SMILES: *CCO]

In some embodiments, -L₄-R₄ is

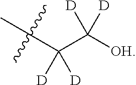 [SMILES: *C([2H])([2H])C([2H])([2H])O]

In some embodiments, -L₄-R₄ is

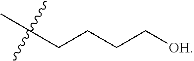 [SMILES: *CCCCO]

In some embodiments, -L₄-R₄ is

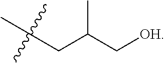 [SMILES: *CC(C)CO]

In some embodiments, -L₄-R₄ is

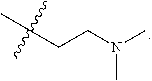 [SMILES: *CCN(C)C]

In some embodiments, -L₄-R₄ is

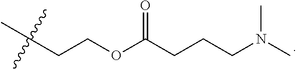 [SMILES: *CCOC(=O)CCCN(C)C]

In some embodiments, -L₄-R₄ is

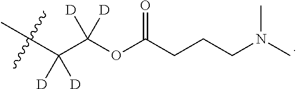 [SMILES: *C([2H])([2H])C([2H])([2H])OC(=O)CCCN(C)C]

In some embodiments, -L₄-R₄ is

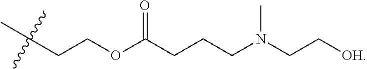 [SMILES: *CCOC(=O)CCCN(C)CCO]

In some embodiments, -L₄-R₄ is

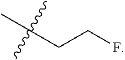 [SMILES: *CCF]

In some embodiments, n is 0, 1, 2, 3, 4, 5, 6, 7, or 8. In some embodiments, n is 2, 3, 4, 5 or 6. In some embodiments, n is 3, 4, 5 or 6. In some embodiments, n is 0, 1, 2, 3, 4 or 5. In some embodiments, n is 0. In some embodiments, n is 1. In some embodiments, n is 2. In some embodiments, n is 3. In some embodiments, n is 4. In some embodiments, n is 5.

In some embodiments, each R¹⁰ is independently hydrogen or C₁₋₁₀ alkyl. In some embodiments, each R¹⁰ is independently hydrogen, C₁₋₁₀ alkyl, C₁₋₁₀ heteroalkyl, C₁₋₁₀ haloalkyl, or C₃₋₁₀ cycloalkyl. In some embodiments, each R¹⁰ is independently hydrogen, C₁₋₆ alkyl, C₁₋₁₀ heteroalkyl, C₁₋₁₀ haloalkyl, or C₃₋₁₀ cycloalkyl. In some embodiments, each R¹⁰ is independently hydrogen or C₁₋₆ alkyl. In some embodiments, each R¹⁰ is independently hydrogen or methyl.

In some embodiments, each R¹¹ is independently hydrogen, deuterium, halogen, —CN, C₁₋₁₀ alkyl, C₁₋₁₀ heteroalkyl, C₁₋₁₀ haloalkyl, C₃₋₁₀ cycloalkyl, C₃₋₁₀ heterocycloalkyl, C₂₋₆ alkenyl, C₂₋₆ alkynyl, —C(O)OH, —C(O)—O—C₁₋₆ alkyl, —C(O)—C₁₋₆ alkyl, —C(O)NH₂, —C(O)NH(C₁₋₆ alkyl), —C(O)N(C₁₋₆ alkyl)₂, —NH₂, —NH(C₁₋₆ alkyl), —N(C₁₋₆ alkyl)₂, —OH, —O—C₁₋₆ alkyl, —SH, —S(C₁₋₆ alkyl), —S(O)(C₁₋₆ alkyl), —S(O)₂(C₁₋₆ alkyl), or —S(O)₂NH(C₁₋₆ alkyl). In some embodiments, each R¹¹ is independently hydrogen, deuterium, halogen, C₁₋₆ alkyl, C₁₋₆ heteroalkyl, C₁₋₆ haloalkyl, C₃₋₆ cycloalkyl, —C(O)OH, —C(O)—O—C₁₋₆ alkyl, —C(O)—C₁₋₆ alkyl, —C(O)NH₂, —C(O)NH(C₁₋₆ alkyl), —C(O)N(C₁₋₆ alkyl)₂, —NH₂, —NH(C₁₋₆ alkyl), —N(C₁₋₆ alkyl)₂, —OH, —O—C₁₋₆ alkyl, —SH, —S(C₁₋₆ alkyl), —S(O)(C₁₋₆ alkyl), —S(O)₂(C₁₋₆ alkyl), or —S(O)₂NH(C₁₋₆ alkyl). In some embodiments, each R¹¹ is independently hydrogen, deuterium, halogen, or C₁₋₆ alkyl.

In some embodiments, described herein is a compound of Formula VI, or an N-oxide, or a pharmaceutically acceptable salt thereof:

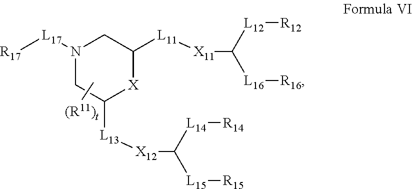

wherein:
X is O, S, or C(R¹¹)₂;
L₁₁ is substituted or unsubstituted linear —C₃₋₁₂ alkylene, wherein one or more methylene units of the group are optionally and independently replaced with —CR¹¹═CR¹¹—;
L₁₂ is substituted or unsubstituted linear —C₃₋₁₂ alkylene, wherein one or more methylene units of the group are optionally and independently replaced with —CR¹¹═CR¹¹—,
L₁₃ is substituted or unsubstituted linear —C₃₋₁₂ alkylene, wherein one or more methylene units of the group are optionally and independently replaced with —CR¹¹═CR¹¹—;
L₁₄ is substituted or unsubstituted linear —C₃₋₁₂ alkylene, wherein one or more methylene units of the group are optionally and independently replaced with —CR¹¹═CR¹¹—;
L₁₅ is substituted or unsubstituted linear —C₀₋₁₂ alkylene, wherein one or more methylene units of the group are optionally and independently replaced with —CR¹¹═CR¹¹—;
L₁₆ is substituted or unsubstituted linear —C₀₋₁₂ alkylene, wherein one or more methylene units of the group are optionally and independently replaced with —CR¹¹═CR¹¹—;
wherein if L₁₁, L₁₂, L₁₃, L₁₄, L₁₅, or L₁₆ is substituted, then L₁₁, L₁₂, L₁₃, L₁₄, L₁₅, or L₁₆ is substituted with 1-5 substituents selected from the group consisting of halogen, deuterium, —CN, linear or branched C₁₋₆ alkyl, C₁₋₂ haloalkyl, —C(═O)R¹⁰, —C(═O)N(R¹⁰)₂, —OR¹⁰, —N(R¹⁰)₂, substituted or unsubstituted C₃₋₆ cycloalkyl, and substituted or unsubstituted 3- to 6-membered heterocycloalkyl; wherein each substituted C₃₋₆ cycloalkyl and substituted 3- to 6-membered heterocycloalkyl is substituted with 1-5 R₁₁;
X₁₁ is a covalent bond, —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—;
X₁₂ is a covalent bond, —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—;
each R¹², R¹⁴, R₁₅ and R¹⁶, is independently hydrogen, deuterium, halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl;
L₁₇ is substituted or unsubstituted —C₁₋₂₄ alkylene-; wherein Liz is optionally substituted with 1 to 10 R¹¹;
R₁₇ is —OH, —OR¹⁰, —O—C(═O)—R¹⁰, —C(═O)—OR¹⁰, —O—C(═O)—OR¹⁰—, —C(═O)—O—C(═O)—R¹⁰, —O—C(═O)—C₀₋₉ alkylene-R¹¹, —(O—C₀₋₉ alkylene-)ₙR¹¹, —O—C(═O)—C₀₋₉ alkylene-N(R¹⁰)₂, —N(R¹⁰)₂, —CH(CH₃)—CH₂—OH, or —C(═O)—C₀₋₉ alkylene-N(R¹⁰)₂;
each R¹⁰ is independently hydrogen, C₁₋₁₀ alkyl, C₁₋₁₀ heteroalkyl, C₁₋₁₀ haloalkyl, C₃₋₁₀ heterocycloalkyl, C₂₋₆ alkenyl, C₂₋₆ alkynyl or C₃₋₁₀ cycloalkyl;
each R¹¹ is independently selected from the group consisting of hydrogen, deuterium, halogen, —CN, —C₁₋₆ alkyl, C₁₋₁₀ heteroalkyl, C₁₋₁₀ haloalkyl, C₃₋₁₀ heterocycloalkyl, C₂₋₆ alkenyl, C₂₋₆ alkynyl, —C(O)OH, —C(O)—O—C₁₋₆ alkyl, —C(O)—C₁₋₆ alkyl, —C(O)NH₂, —C(O)NH(C₁₋₆ alkyl), —C(O)N(C₁₋₆ alkyl)₂, —NH₂, —NH(C₁₋₆ alkyl), —N(C₁₋₆ alkyl)₂, —OH, —O—C₁₋₆ alkyl, —SH, —S(C₁₋₆ alkyl), —S(O)(C₁₋₆ alkyl), —S(O)₂(C₁₋₆ alkyl), or —S(O)₂NH(C₁₋₆ alkyl); and
t is 0, 1, 2, 3, or 4.

In some embodiments, X is O, S, or C(R¹¹)₂. In some embodiments, X is O. In some embodiments, X is S. In some embodiments, X is C(R¹¹)₂. In some embodiments, X is C(R¹¹)₂ and each R¹¹ is independently hydrogen, deuterium, halogen, —C₁₋₃ alkyl, or C₁₋₃ haloalkyl.

In some embodiments, L₁₁ is substituted or unsubstituted linear —C₃₋₁₂ alkylene, wherein one or more methylene units of the group are optionally and independently replaced with —CR¹¹═CR¹¹—. In some embodiments, L₁₁ is unsubstituted linear —C₃₋₁₂ alkylene, wherein one or more methylene units of the group are optionally and independently replaced with —CR¹¹═CR¹¹. In some embodiments, L₁₁ is substituted or unsubstituted linear C₃₋₁₂ alkylene. In some embodiments, L₁₁ is substituted or unsubstituted linear C₃ alkylene. In some embodiments, L₁₁ is substituted or unsubstituted linear C₄ alkylene. In some embodiments, L₁₁ is substituted or unsubstituted linear C₅ alkylene. In some embodiments, L₁₁ is substituted or unsubstituted linear C₆ alkylene. In some embodiments, L₁₁ is substituted or unsubstituted linear C₇ alkylene. In some embodiments, L₁₁ is substituted or unsubstituted linear C₈ alkylene. In some embodiments, L₁₁ is substituted or unsubstituted linear C₉ alkylene. In some embodiments, Ln is substituted or unsubstituted linear C₁₀ alkylene. In some embodiments, L₁₁ is substituted or unsubstituted linear C₁₁ alkylene. In some embodiments. L₁₁ is substituted or unsubstituted linear C₁₂ alkylene. In some embodiments, L₁₁ is

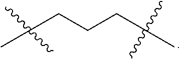 [SMILES: *CCC*]

In some embodiments, L₁₁ is

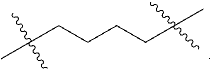 [SMILES: *CCCC*]

In some embodiments, L₁₁ is

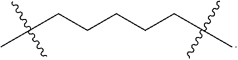 [SMILES: *CCCCC*]

In some embodiments, L₁₁ is

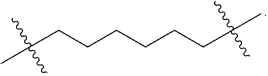 [SMILES: *CCCCCC*]

In some embodiments, L₁₁ is

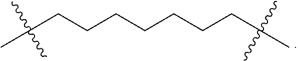 [SMILES: *CCCCCCC*]

In some embodiments, L₁₁ is

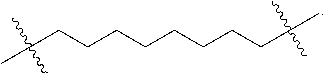 [SMILES: *CCCCCCCC*]

In some embodiments, L₁₁ is

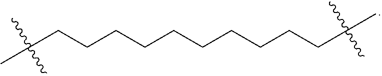 [SMILES: *CCCCCCCCCC*]

In some embodiments, one methylene unit of L₁₁ is with —CR¹¹═CR¹¹—. In some embodiments, one methylene unit of L₁₁ is replaced with —CH═CH—. In some embodiments, L₁₁ is

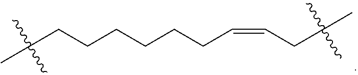 [SMILES: *C/C=C\CCCCCC*]

In some embodiments, L₁₁ is substituted with 1-5 substituents selected from the group consisting of halogen, deuterium, —CN, halogen, —C₁₋₃ alkyl, and C₁₋₃ haloalky.

In some embodiments, L₁₂ is substituted or unsubstituted linear —C₃₋₁₂ alkylene, wherein one or more methylene units of the group are optionally and independently replaced with —CR¹¹═CR¹¹—. In some embodiments, L₁₂ is unsubstituted linear —C₃₋₁₂ alkylene, wherein one or more methylene units of the group are optionally and independently replaced with —CR¹¹═CR¹¹—. In some embodiments, L₁₂ is substituted or unsubstituted linear C₃₋₁₂ alkylene. In some embodiments, L₁₂ is substituted or unsubstituted linear C₃ alkylene. In some embodiments, L₁₂ is substituted or unsubstituted linear C₄ alkylene. In some embodiments, L₁₂ is substituted or unsubstituted linear C₅ alkylene. In some embodiments, L₁₂ is substituted or unsubstituted linear C₆ alkylene. In some embodiments, L₁₂ is substituted or unsubstituted linear C₇ alkylene. In some embodiments, L₁₂ is substituted or unsubstituted linear C₈ alkylene. In some embodiments, L₁₂ is substituted or unsubstituted linear C₉ alkylene. In some embodiments, L₁₂ is substituted or unsubstituted linear C₁₀ alkylene. In some embodiments, L₁₂ is substituted or unsubstituted linear C₁₁ alkylene. In some embodiments, L₁₂ is substituted or unsubstituted linear C₁₂ alkylene. In some embodiments, L₁₂ is

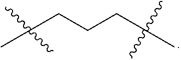 [SMILES: *CCC*]

In some embodiments, L₁₂ is

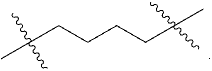 [SMILES: *CCCC*]

In some embodiments, L₁₂ is

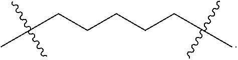 [SMILES: *CCCCC*]

In some embodiments, L₁₂ is

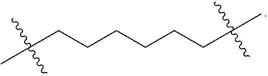 [SMILES: *CCCCCC*]

In some embodiments, L₁₂ is

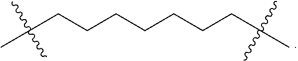 [SMILES: *CCCCCCC*]

In some embodiments, L₁₂ is

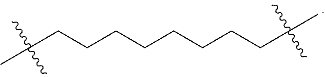 [SMILES: *CCCCCCCC*]

In some embodiments, L₁₂ is

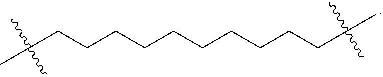 [SMILES: *CCCCCCCCCC*]

In some embodiments, one methylene unit of L₁₂ is replaced with —CR¹¹═CR¹¹—. In some embodiments, one methylene unit of L₁₂ is replaced with —CH═CH—. In some embodiments, L₁₂ is

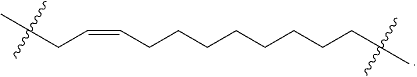 [SMILES: *C/C=C\CCCCCCCC*]

In some embodiments, L₁₂ is substituted with 1-5 substituents selected from the group consisting of halogen, deuterium, —CN, halogen, —C₁₋₃ alkyl, and C₁₋₃ haloalky.

In some embodiments, L₁₃ is substituted or unsubstituted linear —C₃₋₁₂ alkylene, wherein one or more methylene units of the group are optionally and independently replaced with —CR¹¹═CR¹¹—. In some embodiments, L₁₃ is unsubstituted linear —C₃₋₁₂ alkylene, wherein one or more methylene units of the group are optionally and independently replaced with —CR¹¹═CR¹¹—. In some embodiments, L₁₃ is substituted or unsubstituted linear C₃₋₁₂ alkylene. In some embodiments, L₁₃ is substituted or unsubstituted linear C₃ alkylene. In some embodiments, L₁₃ is substituted or unsubstituted linear C₄ alkylene. In some embodiments, L₁₃ is substituted or unsubstituted linear C₅ alkylene. In some embodiments, L₁₃ is substituted or unsubstituted linear C₆ alkylene. In some embodiments, L₁₃ is substituted or unsubstituted linear C₇ alkylene. In some embodiments, L₁₃ is substituted or unsubstituted linear C₈ alkylene. In some embodiments, L₁₃ is substituted or unsubstituted linear C₉ alkylene. In some embodiments, L₁₃ is substituted or unsubstituted linear C₁₀ alkylene. In some embodiments, L₁₃ is substituted or unsubstituted linear C₁₁ alkylene. In some embodiments. L₁₃ is substituted or unsubstituted linear C₁₂ alkylene. In some embodiments, L₁₃ is

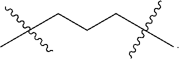 [SMILES: *CCC*]

In some embodiments, L₁₃ is

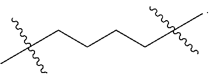 [SMILES: *CCCC*]

In some embodiments, L₁₃ is

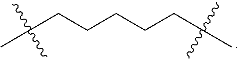 [SMILES: *CCCCC*]

In some embodiments, L₁₃ is

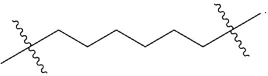 [SMILES: *CCCCCC*]

In some embodiments, L₁₃ is

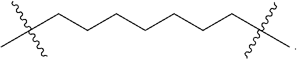 [SMILES: *CCCCCCC*]

In some embodiments, L₁₃ is

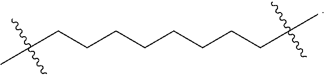 [SMILES: *CCCCCCCC*]

In some embodiments, L₁₃ is

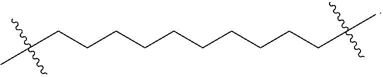 [SMILES: *CCCCCCCCCC*]

In some embodiments, one methylene unit of L₁₃ is replaced with —CR¹¹═CR¹¹—. In some embodiments, one methylene unit of L₁₃ is replaced with —CH═CH—. In some embodiments, L₁₃ is

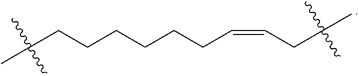 [SMILES: *C/C=C\CCCCCC*]

In some embodiments, L₁₃ is substituted with 1-5 substituents selected from the group consisting of halogen, deuterium, —CN, halogen, —C₁₋₃ alkyl, and C₁₋₃ haloalky.

In some embodiments, L₁₄ is substituted or unsubstituted linear —C₃₋₁₂ alkylene, wherein one or more methylene units of the group are optionally and independently replaced with —CR¹¹═CR¹¹—. In some embodiments, L₁₄ is unsubstituted linear —C₃₋₁₂ alkylene, wherein one or more methylene units of the group are optionally and independently replaced with —CR¹¹═CR¹¹—. In some embodiments, L₁₄ is substituted or unsubstituted linear C₃₋₁₂ alkylene. In some embodiments, L₁₄ is substituted or unsubstituted linear C₃ alkylene. In some embodiments, L₁₄ is substituted or unsubstituted linear C₄ alkylene. In some embodiments, L₁₄ is substituted or unsubstituted linear C₅ alkylene. In some embodiments, L₁₄ is substituted or unsubstituted linear C₆ alkylene. In some embodiments, L₁₄ is substituted or unsubstituted linear C₇ alkylene. In some embodiments, L₁₄ is substituted or unsubstituted linear C₈ alkylene. In some embodiments. L₁₄ is substituted or unsubstituted linear C₉ alkylene. In some embodiments, L₁₄ is substituted or unsubstituted linear C₁₀ alkylene. In some embodiments, L₁₄ is substituted or unsubstituted linear C₁₁ alkylene. In some embodiments, L₁₄ is substituted or unsubstituted linear C₁₂ alkylene. In some embodiments, L₁₄ is

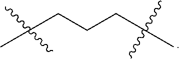 [SMILES: *CCC*]

In some embodiments, L₁₄ is

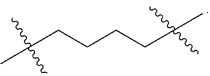 [SMILES: *CCCC*]

In some embodiments, L₁₄ is

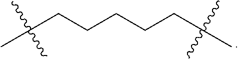 [SMILES: *CCCCC*]

In some embodiments, L₁₄ is

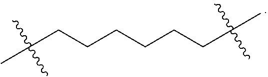 [SMILES: *CCCCCC*]

In some embodiments, L₁₄ is

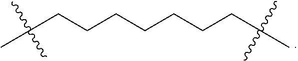 [SMILES: *CCCCCCC*]

In some embodiments, L₁₄ is

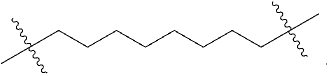 [SMILES: *CCCCCCCC*]

In some embodiments, L₁₄ is

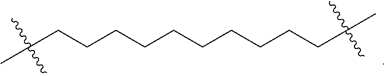 [SMILES: *CCCCCCCCCC*]

In some embodiments, one methylene unit of L₁₄ is replaced with —CR¹¹═CR¹¹—. In some embodiments, one methylene unit of L₁₄ is replaced with —CH═CH—. In some embodiments, L₁₄ is

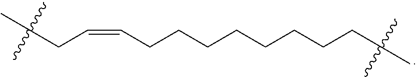 [SMILES: *C/C=C\CCCCCCCC*]

In some embodiments, L₁₄ is substituted with 1-5 substituents selected from the group consisting of halogen, deuterium, —CN, halogen, —C₁₋₃ alkyl, and C₁₋₃ haloalky.

In some embodiments, L₁₅ is substituted or unsubstituted linear —C₀₋₁₂ alkylene, wherein one or more methylene units of the group are optionally and independently replaced with —CR¹¹═CR¹¹—. In some embodiments, L₁₅ is unsubstituted linear —C₃₋₁₂ alkylene, wherein one or more methylene units of the group are optionally and independently replaced with —CR¹¹═CR¹¹—. In some embodiments, L₁₅ is substituted or unsubstituted linear C₃₋₁₂ alkylene. In some embodiments, L₁₅ is a covalent bond when L₁₅ is C₀ alkylene. In some embodiments, L₁₅ is substituted or unsubstituted linear C₂ alkylene. In some embodiments, L₁₅ is substituted or unsubstituted linear C₃ alkylene. In some embodiments, L₁₅ is substituted or unsubstituted linear C₄ alkylene. In some embodiments, L₁₅ is substituted or unsubstituted linear C₅ alkylene. In some embodiments, L₁₅ is substituted or unsubstituted linear C₆ alkylene. In some embodiments, L₁₅ is substituted or unsubstituted linear C₇ alkylene. In some embodiments, Lis is substituted or unsubstituted linear C₈ alkylene. In some embodiments, L₁₅ is substituted or unsubstituted linear C₉ alkylene. In some embodiments, L₁₅ is substituted or unsubstituted linear C₁₀ alkylene. In some embodiments, L₁₅ is substituted or unsubstituted linear C₁₁ alkylene. In some embodiments, L₁₅ is substituted or unsubstituted linear C₁₂ alkylene. In some embodiments, L₁₅ is

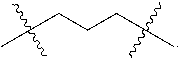 [SMILES: *CCC*]

In some embodiments, L₁₅ is

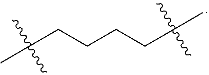 [SMILES: *CCCC*]

In some embodiments, L₁₅ is

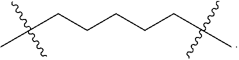 [SMILES: *CCCCC*]

In some embodiments, L₁₅ is

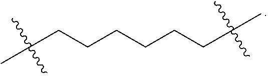 [SMILES: *CCCCCC*]

In some embodiments, L₁₅ is

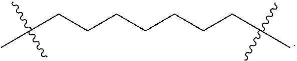 [SMILES: *CCCCCCC*]

In some embodiments, L₁₅ is

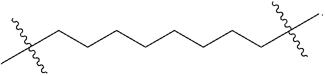 [SMILES: *CCCCCCCC*]

In some embodiments, L₁₅ is

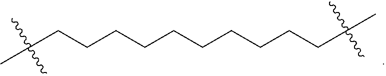 [SMILES: *CCCCCCCCCC*]

In some embodiments, one methylene unit of L₁₅ is replaced with —CR¹¹═CR¹¹—. In some embodiments, one methylene unit of L₁₅ is replaced with —CH═CH—. In some embodiments, L₁₅ is

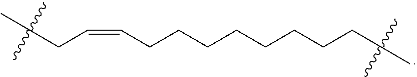 [SMILES: *C/C=C\CCCCCCCC*]

In some embodiments, L₁₅ is substituted with 1-5 substituents selected from the group consisting of halogen, deuterium, —CN, halogen, —C₁₋₃ alkyl, and C₁₋₃ haloalky.

In some embodiments, L₁₆ is substituted or unsubstituted linear —C₀₋₁₂ alkylene, wherein one or more methylene units of the group are optionally and independently replaced with —CR¹¹═CR¹¹—. In some embodiments, L₁₆ is unsubstituted linear —C₃₋₁₂ alkylene, wherein one or more methylene units of the group are optionally and independently replaced with —CR¹¹═CR¹¹—. In some embodiments, L₁₆ is substituted or unsubstituted linear C₃₋₁₂ alkylene. In some embodiments. L₁₆ is a covalent bond when L₁₆ is C₀ alkylene. In some embodiments, L₁₆ is substituted or unsubstituted linear C₂ alkylene. In some embodiments, L₁₆ is substituted or unsubstituted linear C₃ alkylene. In some embodiments, L₁₆ is substituted or unsubstituted linear C₄ alkylene. In some embodiments, L₁₆ is substituted or unsubstituted linear C₅ alkylene. In some embodiments, L₁₆ is substituted or unsubstituted linear C₆ alkylene. In some embodiments, L₁₆ is substituted or unsubstituted linear C₇ alkylene. In some embodiments, L₁₆ is substituted or unsubstituted linear C₈ alkylene. In some embodiments, L₁₆ is substituted or unsubstituted linear C₉ alkylene. In some embodiments, L₁₆ is substituted or unsubstituted linear C₁₀ alkylene. In some embodiments, L₁₆ is substituted or unsubstituted linear C₁₁ alkylene. In some embodiments, L₁₆ is substituted or unsubstituted linear C₁₂ alkylene. In some embodiments, L₁₆ is

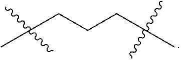 [SMILES: *CCC*]

In some embodiments, L₁₆ is

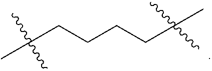 [SMILES: *CCCC*]

In some embodiments, L₁₆ is

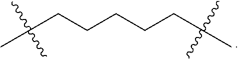 [SMILES: *CCCCC*]

In some embodiments, L₁₆ is

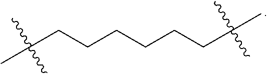 [SMILES: *CCCCCC*]

In some embodiments, L₁₆ is

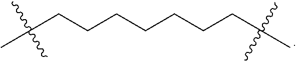 [SMILES: *CCCCCCC*]

In some embodiments, L₁₆ is

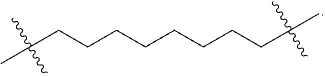 [SMILES: *CCCCCCCC*]

In some embodiments, L₁₆ is

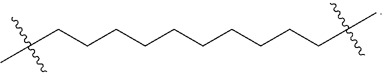 [SMILES: *CCCCCCCCCC*]

In some embodiments, one methylene unit of L₁₆ is replaced with —CR¹¹═CR¹¹—. In some embodiments, one methylene unit of L₁₆ is replaced with —CH═CH—. In some embodiments, L₁₆ is

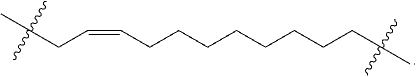 [SMILES: *C/C=C\CCCCCCCC*]

In some embodiments, L₁₆ is substituted with 1-5 substituents selected from the group consisting of halogen, deuterium, —CN, halogen, —C₁₋₃ alkyl, and C₁₋₃ haloalky.

In some embodiments, X₁₁ is a covalent bond, —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—. In some embodiments, X₁₁ is a covalent bond. In some embodiments, X₁₁ is —C(═O)—O—, —O—C(═O)—, —O—C(═O)—O—, —C(═O)—N(R¹⁰)—, or —N(R¹⁰)—C(═O)—. In some embodiments, X₁₁ is —C(═O)—O— or —O—C(═O)—. In some embodiments, X₁₁ is —C(═O)—O—, —O—C(═O)—, —S—S—, —C(═O)—S—, or —C(C═S)—O—. In some embodiments, X₁₁ is —C(═O)—O—. In some embodiments, X₁₁ is —O—C(═O)—.

In some embodiments, X₁₂ is a covalent bond, —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—. In some embodiments, X₁₂ is a covalent bond. In some embodiments, X₁₂ is —C(═O)—O—, —O—C(═O)—, —O—C(═O)—O—, —C(═O)—N(R¹⁰)—, or —N(R¹⁰)—C(═O)—. In some embodiments, X₁₂ is —C(═O)—O— or —O—C(═O)—. In some embodiments, X₁₂ is —C(═O)—O—, —O—C(═O)—, —S—S—, —C(═O)—S—, or —C(C═S)—O—. In some embodiments, X₁₂ is —C(═O)—O—. In some embodiments, X₁₂ is —O—C(═O)—.

In some embodiments, X₁₁ is a covalent bond and X₁₂ is a covalent bond. In some embodiments, X₁₁ is a covalent bond and X₁₂ is —C(═O)—O— or —O—C(═O)—. In some embodiments, X₁₁ is —C(═O)—O— or —O—C(═O)— and X₁₂ is a covalent bond. In some embodiments, X₁₁ is —C(═O)—O— and X₁₂ is —O—C(═O)—. In some embodiments, X₁₁ is —C(═O)—O— and X₁₂ is —C(═O)—O—. In some embodiments, X₁₁ is —O—C(═O)— and X₁₂ is —O—C(═O)—. In some embodiments, X₁₁ is —O—C(═O)— and X₁₂ is —C(═O)—O—.

In some embodiments, R₁₂ is hydrogen, deuterium, halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl. In some embodiments, R₁₂ is hydrogen. In some embodiments, R₁₂ is deuterium. In some embodiments, R₁₂ is halogen. In some embodiments, R₁₂ is F. In some embodiments, R₁₂ is Cl. In some embodiments, R₁₂ is Br. In some embodiments, R₁₂ is I. In some embodiments, R₁₂ is —CN. In some embodiments, R₁₂ is —OR¹⁰. In some embodiments, R₁₂ is —N(R¹⁰)₂. In some embodiments, R₁₂ is branched C₃₋₁₀ alkyl. In some embodiments, R₁₂ is C₃₋₁₀ cycloalkyl. In some embodiments, R₁₂ is C₂₋₆ alkenyl. In some embodiments, R₁₂ is C₂₋₆ alkynyl.

In some embodiments, R₁₄ is hydrogen, deuterium, halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl. In some embodiments, R₁₄ is hydrogen. In some embodiments, R₁₄ is deuterium. In some embodiments, R₁₄ is halogen. In some embodiments, R₁₄ is F. In some embodiments, R₁₄ is Cl. In some embodiments, R₁₄ is Br. In some embodiments, R₁₄ is I. In some embodiments, R₁₄ is —CN. In some embodiments, R₁₄ is —OR¹⁰. In some embodiments, R₁₄ is —N(R¹⁰)₂. In some embodiments, R₁₄ is branched C₃₋₁₀ alkyl. In some embodiments, R₁₄ is C₃₋₁₀ cycloalkyl. In some embodiments, R₁₄ is C₂₋₆ alkenyl. In some embodiments, R₁₄ is C₂₋₆ alkynyl.

In some embodiments, R₁₅ is hydrogen, deuterium, halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl. In some embodiments, R₁₅ is hydrogen. In some embodiments, R₁₅ is deuterium. In some embodiments, R₁₅ is halogen. In some embodiments, R₁₅ is F. In some embodiments, R₁₅ is Cl. In some embodiments, R₁₅ is Br. In some embodiments, R₁₅ is I. In some embodiments, R₁₅ is —CN. In some embodiments, R₁₅ is —OR¹⁰. In some embodiments, R₁₅ is —N(R¹⁰)₂. In some embodiments, R₁₅ is branched C₃₋₁₀ alkyl. In some embodiments, R₁₅ is C₃₋₁₀ cycloalkyl. In some embodiments, R₁₅ is C₂₋₆ alkenyl. In some embodiments, R₁₅ is C₂₋₆ alkynyl.

In some embodiments, R₁₆ is hydrogen, deuterium, halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl. In some embodiments, R₁₆ is hydrogen. In some embodiments, R₁₆ is deuterium. In some embodiments, R₁₆ is halogen. In some embodiments, R₁₆ is F. In some embodiments, R₁₆ is Cl. In some embodiments, R₁₆ is Br. In some embodiments, R₁₆ is I. In some embodiments, R₁₆ is —CN. In some embodiments, R₁₆ is —OR¹⁰. In some embodiments, R₁₆ is —N(R¹⁰)₂. In some embodiments, R₁₆ is branched C₃₋₁₀ alkyl. In some embodiments, R₁₆ is C₃₋₁₀ cycloalkyl. In some embodiments, R₁₆ is C₂₋₆ alkenyl. In some embodiments, R₁₆ is C₂₋₆ alkynyl.

In some embodiments,

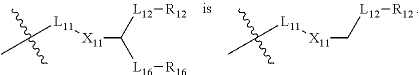

In some embodiments, -L₁₁-X₁₁-L₁₂-R₁₂ is —C₃₋₁₀ alkylene-C(═O)—O—C₃₋₁₀ alkylene-H. In some embodiments, -L₁₁-X₁₁-L₁₂-R₁₂ is —C₃₋₁₀ alkylene-O—C(═O)—C₃₋₁₀ alkylene-H. In some embodiments, -L₁₁-X₁₁-L₁₂-R₁₂ is —C₃₋₁₀ alkylene-CH═CH—C₃₋₁₀ alkylene-H. In some embodiments,

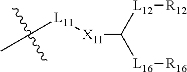

is

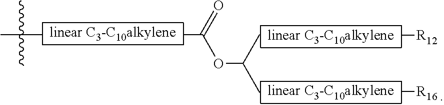

In some embodiments,

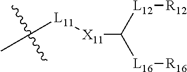

is

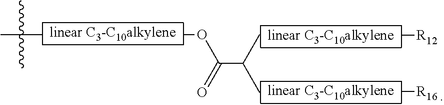

In some embodiments,

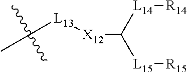

is

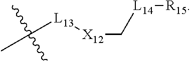

In some embodiments, -L₁₃-X₁₂-L₁₄-R₁₅ is —C₃₋₁₀ alkylene-C(═O)—O—C₃₋₁₀ alkylene-H. In some embodiments, -L₁₃-X₁₂-L₁₄-R₁₅ is —C₃₋₁₀ alkylene-O—C(═O)—C₃₋₁₀ alkylene-H. In some embodiments, -L₁₃-X₁₂-L₁₄-R₁₅ is —C₃₋₁₀ alkylene-CH═CH—C₃₋₁₀ alkylene-H. In some embodiments,

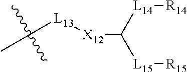

is

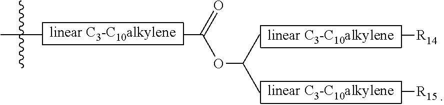

In some embodiments,

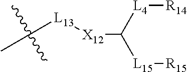

is

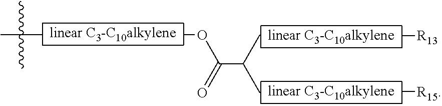

In some embodiments, L₁₇ is substituted or unsubstituted —C₁₋₁₂ alkylene; wherein L₁₇ is optionally substituted with 1 to 10 R¹¹. In some embodiments, L₁₇ is substituted or unsubstituted —C₃₋₂₄ alkenylalkylene, wherein L₁₇ is optionally substituted with 1 to 10 R¹¹. In some embodiments, L₁₇ is substituted or unsubstituted —C₃₋₁₂ alkenylalkylene. In some embodiments, L₁₇ is substituted or unsubstituted —C₃₋₆ alkenylalkylene. In some embodiments, L₁₇ is substituted or unsubstituted —C₁₋₆ alkylene; wherein L₁₇ is optionally substituted with 1 to 10 R¹¹. In some embodiments, L₁₇ is —CH₂—, —CH₂—CH₂—, —CH₂—CH₂—CH₂—, —CH₂—(CH₂)₂—CH₂—, —CH₂—(CH₂)₃—CH₂—, or —CH₂—(CH₂)₄—CH₂—. In some embodiments, L₁₇ is —CH₂—, —CH₂—CH₂—, or —CH₂—CH₂—CH₂—. In some embodiments, L₁₇ is —CH₂—. In some embodiments, L₁₇ is —CH₂—CH₂—. In some embodiments. L₁₇ is —CH₂—CH₂—CH₂—. In some embodiments, L₁₇ is —CH₂—CH(CH₃)—CH₂—. In some embodiments, L₁₇ is —CD₂—CD₂—.

In some embodiments, R₁₇ is —OH, —OR¹⁰, —O—C(═O)—R¹⁰, —C(═O)—OR¹⁰, —O—C(═O)—C₀₋₉ alkylene-N(R¹¹)₂, —O—C(═O)—C₀₋₉ alkylene-R¹¹, —(O—C₀₋₉ alkylene-)ₙR¹¹, —CH(CH₃)—CH₂—OH, or —C(═O)—C₀₋₉ alkylene-N(R¹¹)₂. In some embodiments, R₁₇ is —OH, —CH(CH₃)—CH₂—OH, or —O—C(═O)—(CH₂)₃—N(Me)₂. In some embodiments, R₁₇ is —OH or —O—C(═O)—C₂-4 alkylene-N(R¹¹)₂. In some embodiments, R₁₇ is —OH or —O—C(═O)—C₃₋₆ alkylene-N(R¹¹)₂. In some embodiments, R₁₇ is —OH. In some embodiments, R₁₇ is —O—C(═O)—C₃₋₆ alkylene-N(Me)₂. In some embodiments, R₁₇ is halogen. In some embodiments, R₁₇ is F.

In some embodiments, -L₁₇-R₁₇ is

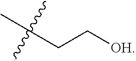 [SMILES: *CCO]

In some embodiments, -L₁₇-R₁₇ is

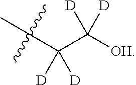 [SMILES: *C([2H])([2H])C([2H])([2H])O]

In some embodiments, -L₁₇-R₁₇ is

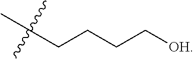 [SMILES: *CCCCO]

In some embodiments, -L₁₇-R₁₇ is

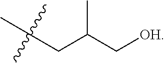 [SMILES: *CC(C)CO]

In some embodiments, -L₁₇-R₁₇ is

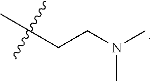 [SMILES: *CCN(C)C]

In some embodiments, -L₁₇-R₁₇ is

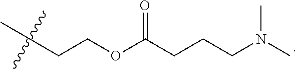 [SMILES: *CCOC(=O)CCCN(C)C]

In some embodiments, -L₁₇-R₁₇ is

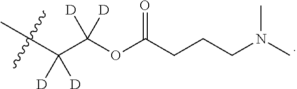 [SMILES: *C([2H])([2H])C([2H])([2H])OC(=O)CCCN(C)C]

In some embodiments, -L₁₇-R₁₇ is

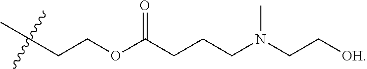 [SMILES: *CCOC(=O)CCCN(C)CCO]

In some embodiments, -L₁₇-R₁₇ is

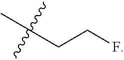 [SMILES: *CCF]

In some embodiments, t is 0. In some embodiments, t is 1. In some embodiments, tis 2. In some embodiments, t is 3. In some embodiments, t is 4.

Any combination of the groups described above for the various variables is contemplated herein. Throughout the specification, groups and substituents thereof are chosen by one skilled in the field to provide stable moieties and compounds.

Exemplary compounds of Formula I′, Formula I′-a, Formula I, Formula I-a, Formula I-b, Formula II, Formula III, Formula III-a, Formula IV, Formula IV-a, Formula V, or Formula VI include compounds described in Table 1, Table 2, and Table 3.

Described herein are nanoparticle compositions comprising a compound described herein. In some embodiments, the nanoparticle composition is a lipid nanoparticle (LNP). In some embodiments, the LNP comprises an amino lipid, a phospholipid, a PEG-lipid, a cholesterol, or a derivative thereof, a payload, or any combination thereof. In some embodiments, the LNP composition comprises an amino lipid. In some embodiments, the LNP composition comprises an amino lipid of having the structure of Formula I′, Formula I′-a, Formula I, Formula I-a, Formula I-b, Formula II, Formula III, Formula III-a, Formula IV, Formula IV-a, or Formula V, or a pharmaceutically acceptable salt or solvate thereof. In some embodiments, the LNP comprises a plurality of amino lipids. For example, the LNP composition can comprise 2, 3, 4, 5, 6, 7, 8, 9, 10, or more amino lipids. For another example, the LNP composition can comprise at least 2, at least 3, at least 4, at least 5, at least 6, at least 7, at least 9, at least 10, or at least 20 amino lipids. For yet another example, the LNP composition can comprise at most 2, at most 3, at most 4, at most 5, at most 6, at most 7, at most 9, at most 10, at most 20, or at most 30 amino lipids.

In some embodiments, an LNP composition described herein comprises a first amino lipid. In some embodiments, the LNP composition comprises a first amino lipid and a second amino lipid. In some embodiments, the LNP composition comprises a first amino lipid, a second amino lipid, and a third amino lipid. In some embodiments, the LNP composition comprises a first amino lipid, a second amino lipid, a third amino lipid, and a fourth amino lipid. In some embodiments, the first amino lipid is selected from Tables 1-3. In some embodiments, the second amino lipid is selected from Tables 1-3. In some embodiments, the third amino lipid is selected from Tables 1-3. In some embodiments, the fourth amino lipid is selected from Tables 1-3. In some embodiments, the LNP composition does not comprise a fourth amino lipid. In some embodiments, the LNP composition does not comprise a third amino lipid. In some embodiments, a molar ratio of the first amino lipid to the second amino lipid is from about 0.01 to about 100. In some embodiments, a molar ratio of the first amino lipid to the second amino lipid is from about 0.05 to about 20. In some embodiments, a molar ratio of the first amino lipid to the second amino lipid is from about 0.1 to about 10. In some embodiments, a molar ratio of the first amino lipid to the second amino lipid is from about 0.20 to about 5. In some embodiments, a molar ratio of the first amino lipid to the second amino lipid is from about 0.25 to about 4. In some embodiments, a molar ratio of the first amino lipid to the second amino lipid is about 0.25, about 0.33, about 0.5, about 1, about 2, about 3, or about 4.

In some embodiments, a molar ratio of the first amino lipid: the second amino lipid: the third amino lipid is about 4:1:1. In some embodiments, a molar ratio of the first amino lipid: the second amino lipid: the third amino lipid is about 1:1:1. In some embodiments, a molar ratio of the first amino lipid: the second amino lipid: the third amino lipid is about 2:1:1. In some embodiments, a molar ratio of the first amino lipid: the second amino lipid: the third amino lipid is about 2:2:1. In some embodiments, a molar ratio of the first amino lipid: the second amino lipid: the third amino lipid is about 3:2:1. In some embodiments, a molar ratio of the first amino lipid: the second amino lipid: the third amino lipid is about 3:1:1. In some embodiments, a molar ratio of the first amino lipid: the second amino lipid: the third amino lipid is about 5:1:1. In some embodiments, a molar ratio of the first amino lipid: the second amino lipid: the third amino lipid is about 3:3:1. In some embodiments, a molar ratio of the first amino lipid: the second amino lipid: the third amino lipid is about 4:4:1.

In some embodiments, the LNP composition comprises one or more amino lipids. In some embodiments, the one or more amino lipids comprise from about 40 mol % to about 65 mol % of the total lipid present in the particle. In some embodiments, the one or more amino lipids comprise about 40 mol %, about 41 mol %, about 42 mol %, about 43 mol %, about 44 mol %, about 45 mol %, about 46 mol %, about 47 mol %, about 48 mol %, about 49 mol %, about 50 mol %, about 51 mol %, about 52 mol %, about 53 mol %, about 54 mol %, about 55 mol %, about 56 mol %, about 57 mol %, about 58 mol %, about 59 mol %, about 60 mol %, about 61 mol %, about 62 mol %, about 63 mol %, about 64 mol %, or about 65 mol % of the total lipid present in the particle. In some embodiments, the first amino lipid comprises from about 1 mol % to about 99 mol % of the total amino lipids present in the particle. In some embodiments, the first amino lipid comprises from about 16.7 mol % to about 66.7 mol % of the total amino lipids present in the particle. In some embodiments, the first amino lipid comprises from about 20 mol % to about 60 mol % of the total amino lipids present in the particle. In some embodiments, the amino lipid is an ionizable lipid.

In some embodiments, the disclosed amino lipids can be converted to N-oxides. In some embodiments, N-oxides are formed by a treatment with an oxidizing agent (e.g., 3-chloroperoxybenzoic acid and/or hydrogen peroxides). Accordingly, disclosed herein are N-oxide compounds of the described amino lipids, when allowed by valency and structure, which can be designated as N→0 or N⁺-0⁻. In some embodiments, the nitrogen in the compounds of the disclosure can be converted to N-hydroxy or N-alkoxy. For example, N-hydroxy compounds can be prepared by oxidation of the parent amine by an oxidizing agent such as ra-CPBA. All shown and claimed nitrogen-containing compounds are also considered. Accordingly, also disclosed herein are N-hydroxy and N-alkoxy (e.g., N—OR, wherein R is substituted or unsubstituted C₁-C₆ alkyl, C₁-C₆ alkenyl, C₁-C₆ alkynyl, 3-14-membered carbocycle or 3-14-membered heterocycle) derivatives of the described amino lipids.

In some embodiments, an amino lipid described herein can take the form of a salt, such as a pharmaceutically acceptable salt. All pharmaceutically acceptable salts of the amino lipid are encompassed by this disclosure. As used herein, the term “amino lipid” also includes its pharmaceutically acceptable salts, and its diastereomeric, enantiomeric, and epimeric forms.

In some embodiments, an amino lipid described herein, possesses one or more stereocenters and each stereocenter exists independently in either the R or S configuration. The lipids presented herein include all diastereomeric, enantiomeric, and epimeric forms as well as the appropriate mixtures thereof. The lipids provided herein include all cis, trans, syn, anti, entgegen (E), and zusammen (Z) isomers as well as the appropriate mixtures thereof. In certain embodiments, lipids described herein are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In one aspect, stereoisomers are obtained by stereoselective synthesis.

In some embodiments, the lipids such as the amino lipids are substituted based on the structures disclosed herein. In some embodiments, the lipids such as the amino lipids are unsubstituted. In another embodiment, the lipids described herein are labeled isotopically (e.g. with a radioisotope) or by another other means, including, but not limited to, the use of chromophores or fluorescent moieties, bioluminescent labels, or chemiluminescent labels.

In some embodiments, the compound described herein (e.g., an amino lipid) includes isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present lipids include isotopes of hydrogen, carbon, nitrogen, oxygen, sulfur, fluorine and chlorine, such as, for example, ²H, ³H, ¹³C, ¹⁴C, ¹⁵N, ¹⁸O, ¹⁷O, ³⁵S, ¹⁸F, ³⁶Cl. In one aspect, isotopically-labeled lipids described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

In some embodiments, the asymmetric carbon atom of the amino lipid is present in enantiomerically enriched form. In certain embodiments, the asymmetric carbon atom of the amino lipid has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (S)- or (R)-configuration.

TABLE 1
Exemplary Amino lipids

| Structure | Name |
|---|---|
| 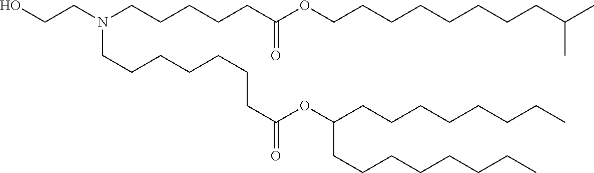 [SMILES: CCCCCCCCC(CCCCCCCC)OC(=O)CCCCCCCN(CCO)CCCCCC(=O)OCCCCCCCCC(C)C] | Compound 1 |
| 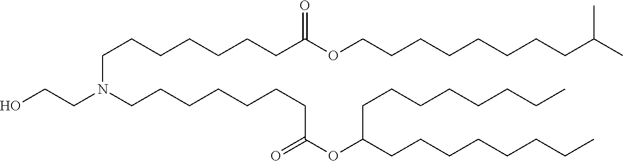 [SMILES: CCCCCCCCC(CCCCCCCC)OC(=O)CCCCCCCN(CCO)CCCCCCCC(=O)OCCCCCCCCC(C)C] | Compound 2 |
| 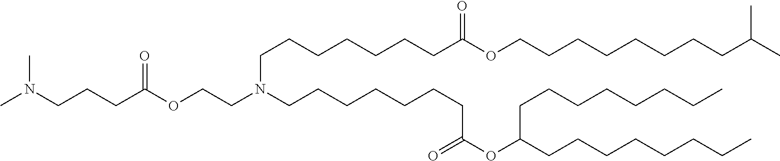 [SMILES: CCCCCCCCC(CCCCCCCC)OC(=O)CCCCCCCN(CCCCCCCC(=O)OCCCCCCCCC(C)C)CCOC(=O)CCCN(C)C] | Compound 3 |
| 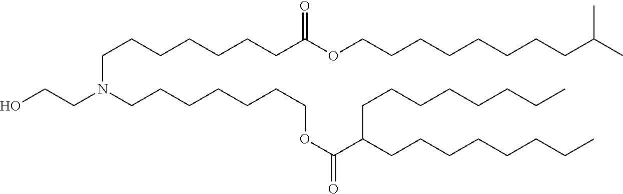 [SMILES: CCCCCCCCC(CCCCCCCC)C(=O)OCCCCCCCN(CCO)CCCCCCCC(=O)OCCCCCCCCC(C)C] | Compound 4 |
| 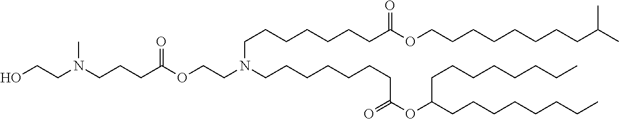 [SMILES: CCCCCCCCC(CCCCCCCC)OC(=O)CCCCCCCN(CCCCCCCC(=O)OCCCCCCCCC(C)C)CCOC(=O)CCCN(C)CCO] | Compound 5 |
| 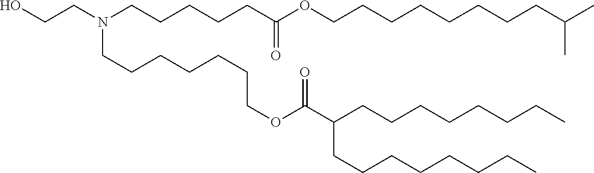 [SMILES: CCCCCCCCC(CCCCCCCC)C(=O)OCCCCCCCN(CCO)CCCCCC(=O)OCCCCCCCCC(C)C] | Compound 6 |
| 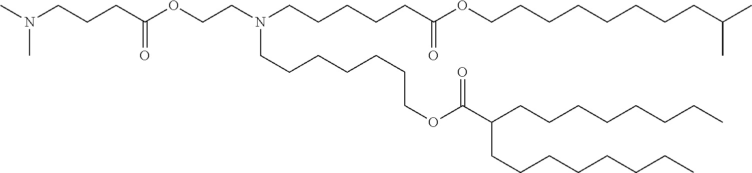 [SMILES: CCCCCCCCC(CCCCCCCC)C(=O)OCCCCCCCN(CCCCCC(=O)OCCCCCCCCC(C)C)CCOC(=O)CCCN(C)C] | Compound 7 |
| 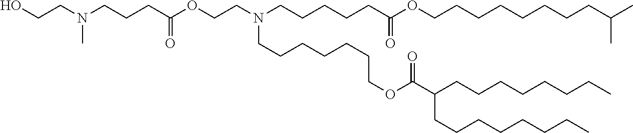 [SMILES: CCCCCCCCC(CCCCCCCC)C(=O)OCCCCCCCN(CCCCCC(=O)OCCCCCCCCC(C)C)CCOC(=O)CCCN(C)CCO] | Compound 8 |
| 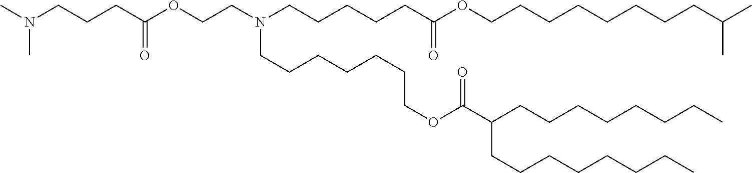 [SMILES: CCCCCCCCC(CCCCCCCC)C(=O)OCCCCCCCN(CCCCCC(=O)OCCCCCCCCC(C)C)CCOC(=O)CCCN(C)C] | Compound 9 |
| 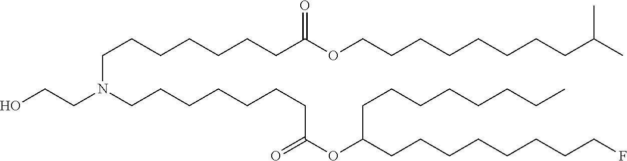 [SMILES: CCCCCCCCC(CCCCCCCCF)OC(=O)CCCCCCCN(CCO)CCCCCCCC(=O)OCCCCCCCCC(C)C] | Compound 10 |
| 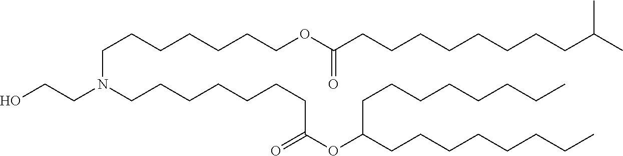 [SMILES: CCCCCCCCC(CCCCCCCC)OC(=O)CCCCCCCN(CCO)CCCCCCCOC(=O)CCCCCCCCC(C)C] | Compound 11 |
| 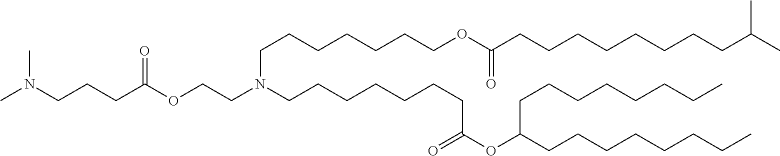 [SMILES: CCCCCCCCC(CCCCCCCC)OC(=O)CCCCCCCN(CCCCCCCOC(=O)CCCCCCCCC(C)C)CCOC(=O)CCCN(C)C] | Compound 12 |
| 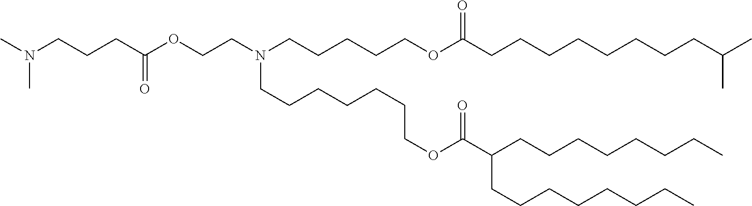 [SMILES: CCCCCCCCC(CCCCCCCC)C(=O)OCCCCCCCN(CCCCCOC(=O)CCCCCCCCC(C)C)CCOC(=O)CCCN(C)C] | Compound 13 |
| 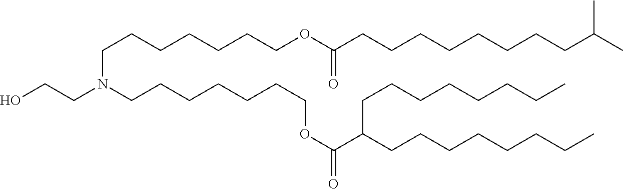 [SMILES: CCCCCCCCC(CCCCCCCC)C(=O)OCCCCCCCN(CCO)CCCCCCCOC(=O)CCCCCCCCC(C)C] | Compound 14 |
| 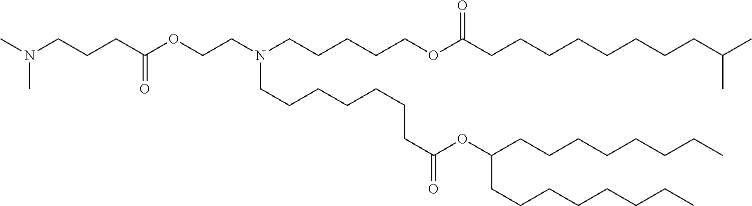 [SMILES: CCCCCCCCC(CCCCCCCC)OC(=O)CCCCCCCN(CCCCCOC(=O)CCCCCCCCC(C)C)CCOC(=O)CCCN(C)C] | Compound 15 |
| 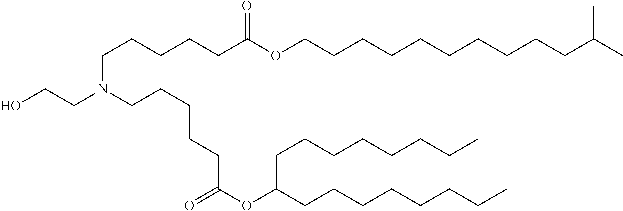 [SMILES: CCCCCCCCC(CCCCCCCC)OC(=O)CCCCCN(CCO)CCCCCC(=O)OCCCCCCCCCCC(C)C] | Compound 16 |
| 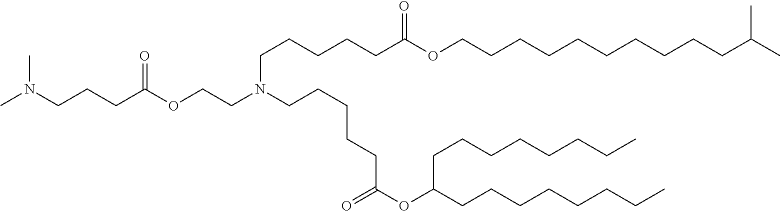 [SMILES: CCCCCCCCC(CCCCCCCC)OC(=O)CCCCCN(CCCCCC(=O)OCCCCCCCCCCC(C)C)CCOC(=O)CCCN(C)C] | Compound 17 |
| 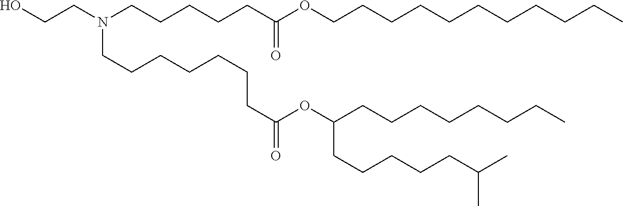 [SMILES: CCCCCCCCCCCOC(=O)CCCCCN(CCO)CCCCCCCC(=O)OC(CCCCCCCC)CCCCCC(C)C] | Compound 18 |
| 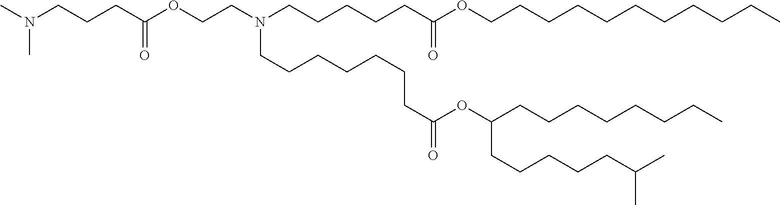 [SMILES: CCCCCCCCCCCOC(=O)CCCCCN(CCCCCCCC(=O)OC(CCCCCCCC)CCCCCC(C)C)CCOC(=O)CCCN(C)C] | Compound 19 |
| 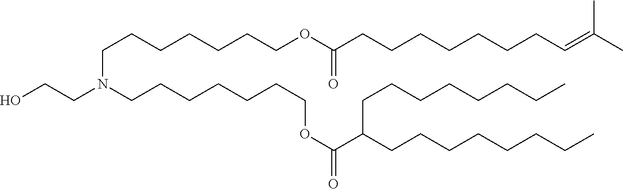 [SMILES: CCCCCCCCC(CCCCCCCC)C(=O)OCCCCCCCN(CCO)CCCCCCCOC(=O)CCCCCCCC=C(C)C] | Compound 20 |
| 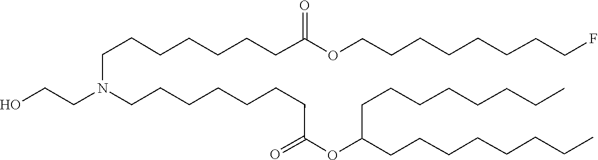 [SMILES: CCCCCCCCC(CCCCCCCC)OC(=O)CCCCCCCN(CCO)CCCCCCCC(=O)OCCCCCCCCF] | Compound 21 |
| 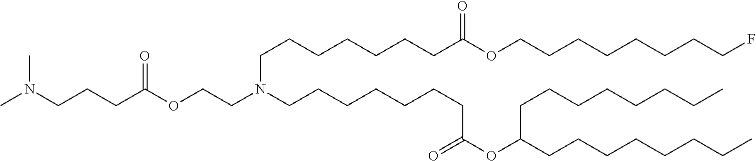 [SMILES: CCCCCCCCC(CCCCCCCC)OC(=O)CCCCCCCN(CCCCCCCC(=O)OCCCCCCCCF)CCOC(=O)CCCN(C)C] | Compound 22 |
| 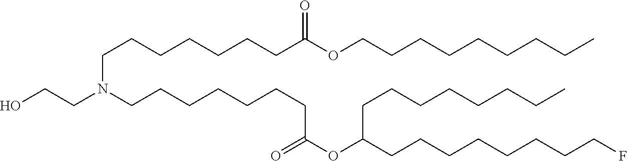 [SMILES: CCCCCCCCCOC(=O)CCCCCCCN(CCO)CCCCCCCC(=O)OC(CCCCCCCC)CCCCCCCCF] | Compound 23 |
| 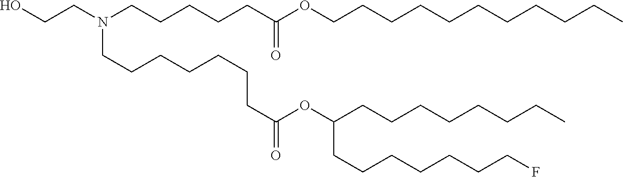 [SMILES: CCCCCCCCCCCOC(=O)CCCCCN(CCO)CCCCCCCC(=O)OC(CCCCCCCC)CCCCCCCF] | Compound 24 |
| 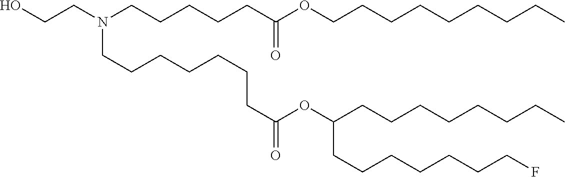 [SMILES: CCCCCCCCCOC(=O)CCCCCN(CCO)CCCCCCCC(=O)OC(CCCCCCCC)CCCCCCCF] | Compound 25 |
| 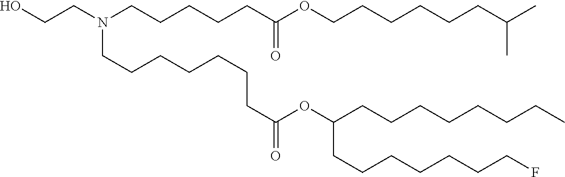 [SMILES: CCCCCCCCC(CCCCCCCF)OC(=O)CCCCCCCN(CCO)CCCCCC(=O)OCCCCCCC(C)C] | Compound 26 |
| 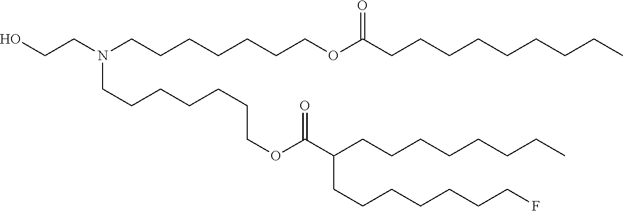 [SMILES: CCCCCCCCCC(=O)OCCCCCCCN(CCO)CCCCCCCOC(=O)C(CCCCCCCC)CCCCCCCF] | Compound 27 |
| 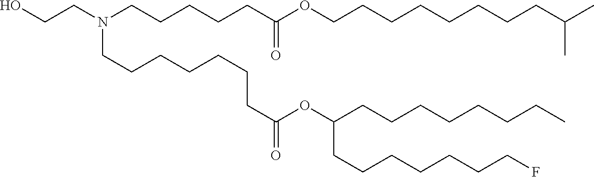 [SMILES: CCCCCCCCC(CCCCCCCF)OC(=O)CCCCCCCN(CCO)CCCCCC(=O)OCCCCCCCCC(C)C] | Compound 28 |
| 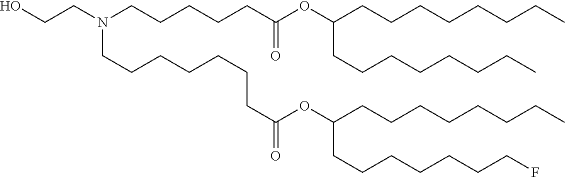 [SMILES: CCCCCCCCC(CCCCCCCF)OC(=O)CCCCCCCN(CCO)CCCCCC(=O)OC(CCCCCCCC)CCCCCCCC] | Compound 29 |
| 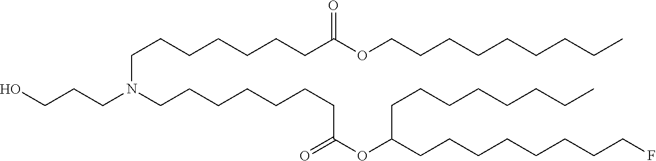 [SMILES: CCCCCCCCCOC(=O)CCCCCCCN(CCCO)CCCCCCCC(=O)OC(CCCCCCCC)CCCCCCCCF] | Compound 30 |
| 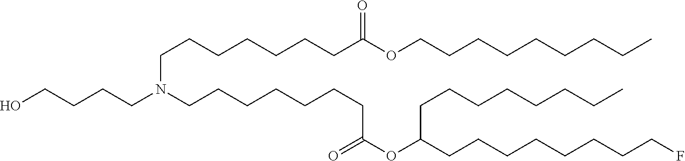 [SMILES: CCCCCCCCCOC(=O)CCCCCCCN(CCCCO)CCCCCCCC(=O)OC(CCCCCCCC)CCCCCCCCF] | Compound 31 |
| 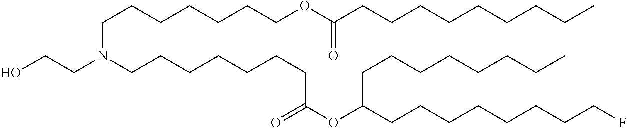 [SMILES: CCCCCCCCCC(=O)OCCCCCCCN(CCO)CCCCCCCC(=O)OC(CCCCCCCC)CCCCCCCCF] | Compound 32 |
| 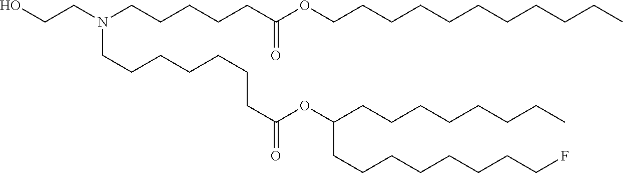 [SMILES: CCCCCCCCCCCOC(=O)CCCCCN(CCO)CCCCCCCC(=O)OC(CCCCCCCC)CCCCCCCCF] | Compound 33 |
| 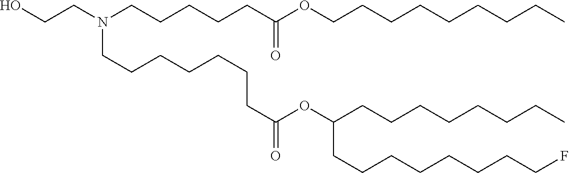 [SMILES: CCCCCCCCCOC(=O)CCCCCN(CCO)CCCCCCCC(=O)OC(CCCCCCCC)CCCCCCCCF] | Compound 34 |
| 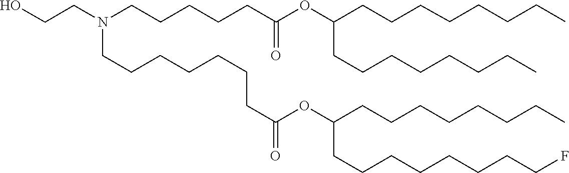 [SMILES: CCCCCCCCC(CCCCCCCCF)OC(=O)CCCCCCCN(CCO)CCCCCC(=O)OC(CCCCCCCC)CCCCCCCC] | Compound 35 |
| 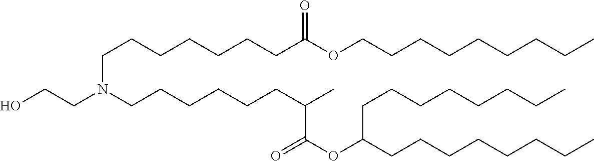 [SMILES: CCCCCCCCCOC(=O)CCCCCCCN(CCO)CCCCCCC(C)C(=O)OC(CCCCCCCC)CCCCCCCC] | Compound 36 |
| 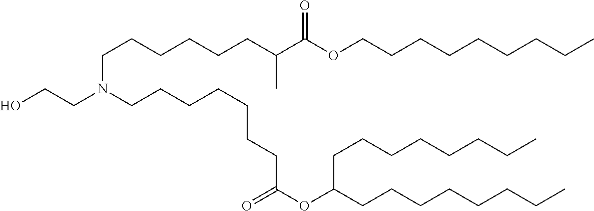 [SMILES: CCCCCCCCCOC(=O)C(C)CCCCCCN(CCO)CCCCCCCC(=O)OC(CCCCCCCC)CCCCCCCC] | Compound 37 |
| 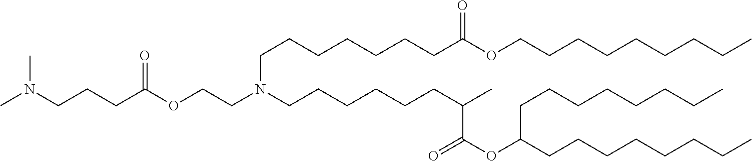 [SMILES: CCCCCCCCCOC(=O)CCCCCCCN(CCCCCCC(C)C(=O)OC(CCCCCCCC)CCCCCCCC)CCOC(=O)CCCN(C)C] | Compound 38 |
| 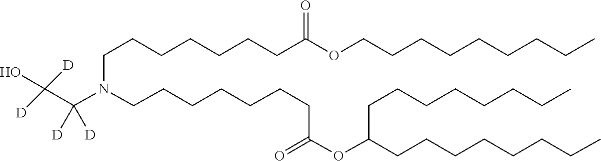 [SMILES: [2H]C([2H])(O)C([2H])([2H])N(CCCCCCCC(=O)OCCCCCCCCC)CCCCCCCC(=O)OC(CCCCCCCC)CCCCCCCC] | Compound 39 |
| 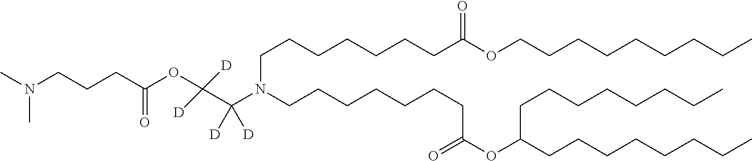 [SMILES: [2H]C([2H])(OC(=O)CCCN(C)C)C([2H])([2H])N(CCCCCCCC(=O)OCCCCCCCCC)CCCCCCCC(=O)OC(CCCCCCCC)CCCCCCCC] | Compound 40 |
| 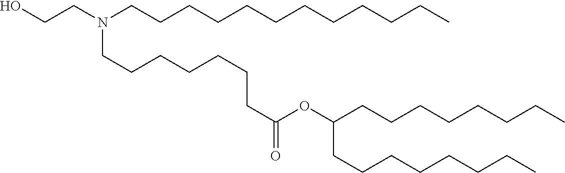 [SMILES: CCCCCCCCCCCCN(CCO)CCCCCCCC(=O)OC(CCCCCCCC)CCCCCCCC] | Compound 41 |
| 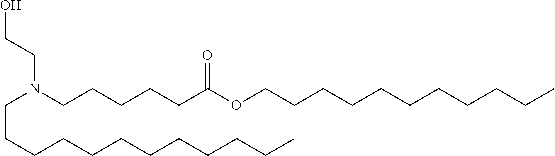 [SMILES: CCCCCCCCCCCCN(CCO)CCCCCC(=O)OCCCCCCCCCCC] | Compound 42 |
| 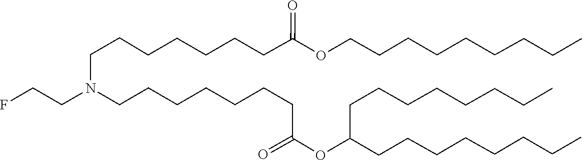 [SMILES: CCCCCCCCCOC(=O)CCCCCCCN(CCF)CCCCCCCC(=O)OC(CCCCCCCC)CCCCCCCC] | Compound 43 |
| 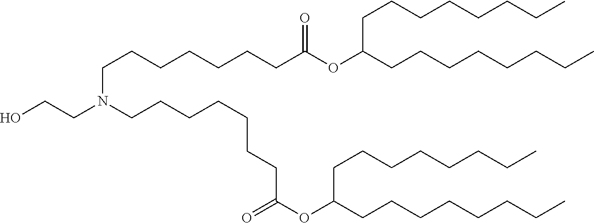 [SMILES: CCCCCCCCC(CCCCCCCC)OC(=O)CCCCCCCN(CCO)CCCCCCCC(=O)OC(CCCCCCCC)CCCCCCCC] | Compound 44 |
| 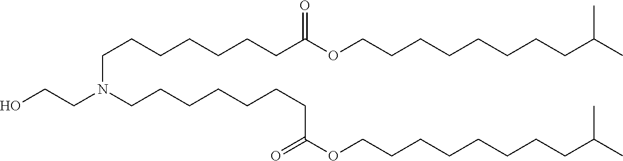 [SMILES: CC(C)CCCCCCCCOC(=O)CCCCCCCN(CCO)CCCCCCCC(=O)OCCCCCCCCC(C)C] | Compound 45 |
| 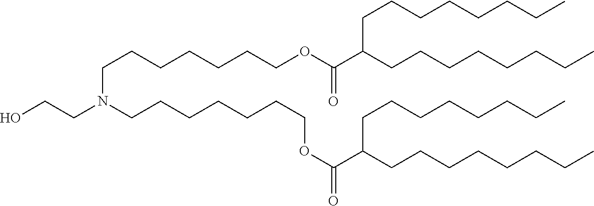 [SMILES: CCCCCCCCC(CCCCCCCC)C(=O)OCCCCCCCN(CCO)CCCCCCCOC(=O)C(CCCCCCCC)CCCCCCCC] | Compound 46 |
| 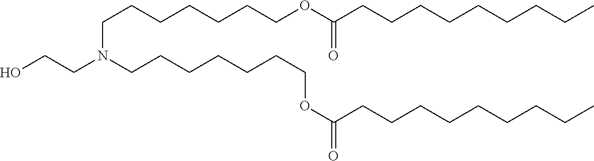 [SMILES: CCCCCCCCCC(=O)OCCCCCCCN(CCO)CCCCCCCOC(=O)CCCCCCCCC] | Compound 47 |
| 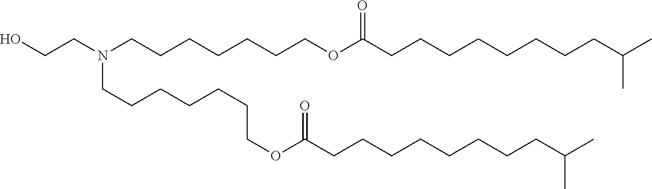 [SMILES: CC(C)CCCCCCCCC(=O)OCCCCCCCN(CCO)CCCCCCCOC(=O)CCCCCCCCC(C)C] | Compound 48 |
| 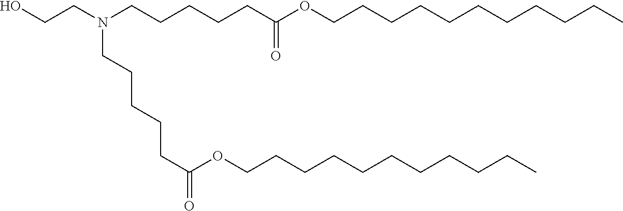 [SMILES: CCCCCCCCCCCOC(=O)CCCCCN(CCO)CCCCCC(=O)OCCCCCCCCCCC] | Compound 49 |
| 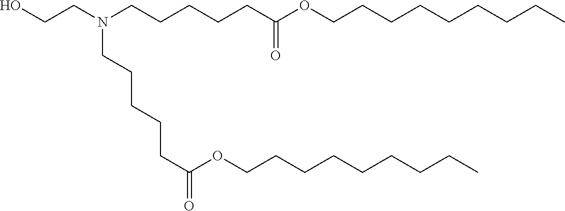 [SMILES: CCCCCCCCCOC(=O)CCCCCN(CCO)CCCCCC(=O)OCCCCCCCCC] | Compound 50 |
| 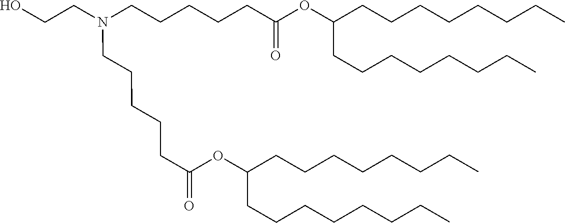 [SMILES: CCCCCCCCC(CCCCCCCC)OC(=O)CCCCCN(CCO)CCCCCC(=O)OC(CCCCCCCC)CCCCCCCC] | Compound 51 |

TABLE 2
Additional Exemplary Amino lipids

| Structure | Name |
|---|---|
| 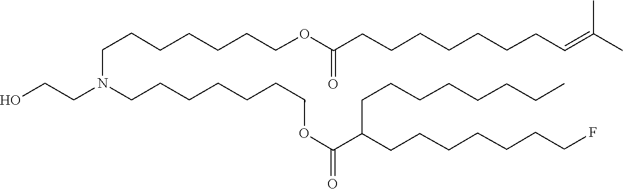 [SMILES: CCCCCCCCC(CCCCCCCF)C(=O)OCCCCCCCN(CCO)CCCCCCCOC(=O)CCCCCCCC=C(C)C] | Compound 52 |
| 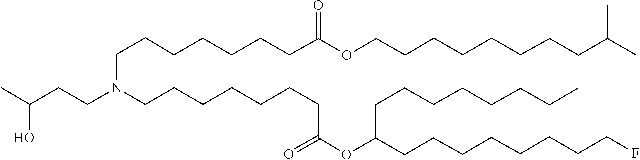 [SMILES: CCCCCCCCC(CCCCCCCCF)OC(=O)CCCCCCCN(CCCCCCCC(=O)OCCCCCCCCC(C)C)CCC(C)O] | Compound 53 |
| 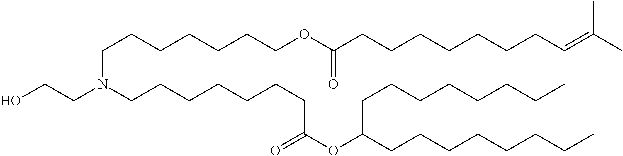 [SMILES: CCCCCCCCC(CCCCCCCC)OC(=O)CCCCCCCN(CCO)CCCCCCCOC(=O)CCCCCCCC=C(C)C] | Compound 54 |
| 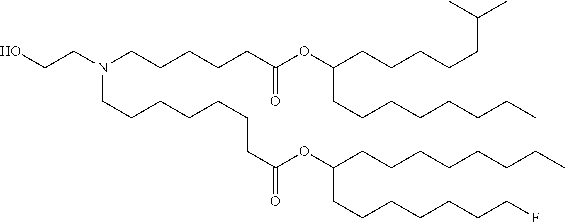 [SMILES: CCCCCCCCC(CCCCCCCF)OC(=O)CCCCCCCN(CCO)CCCCCC(=O)OC(CCCCCCCC)CCCCCC(C)C] | Compound 55 |
| 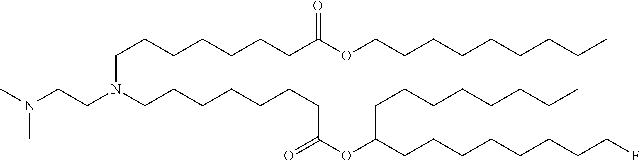 [SMILES: CCCCCCCCCOC(=O)CCCCCCCN(CCCCCCCC(=O)OC(CCCCCCCC)CCCCCCCCF)CCN(C)C] | Compound 56 |
| 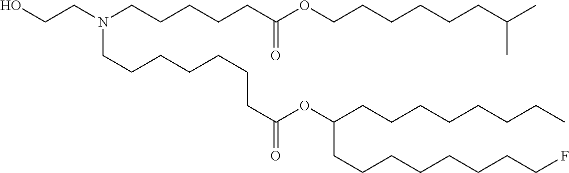 [SMILES: CCCCCCCCC(CCCCCCCCF)OC(=O)CCCCCCCN(CCO)CCCCCC(=O)OCCCCCCC(C)C] | Compound 57 |
| 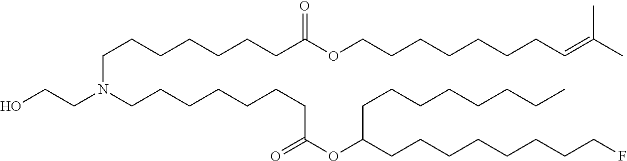 [SMILES: CCCCCCCCC(CCCCCCCCF)OC(=O)CCCCCCCN(CCO)CCCCCCCC(=O)OCCCCCCCC=C(C)C] | Compound 58 |
| 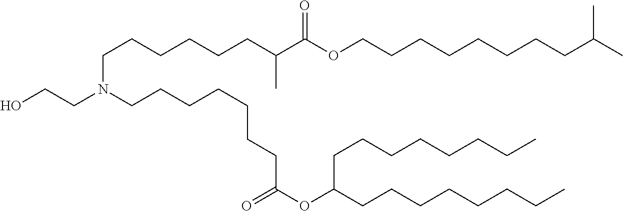 [SMILES: CCCCCCCCC(CCCCCCCC)OC(=O)CCCCCCCN(CCO)CCCCCCC(C)C(=O)OCCCCCCCCC(C)C] | Compound 59 |
| 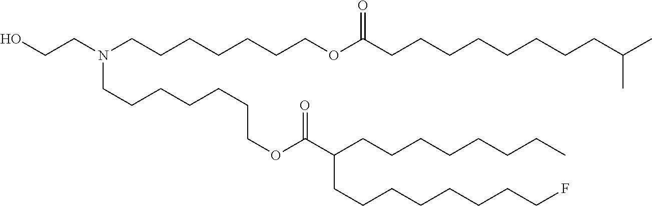 [SMILES: CCCCCCCCC(CCCCCCCCF)C(=O)OCCCCCCCN(CCO)CCCCCCCOC(=O)CCCCCCCCC(C)C] | Compound 60 |
| 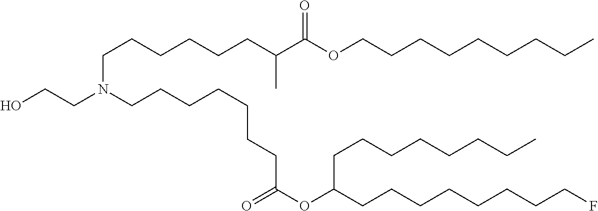 [SMILES: CCCCCCCCCOC(=O)C(C)CCCCCCN(CCO)CCCCCCCC(=O)OC(CCCCCCCC)CCCCCCCCF] | Compound 61 |
| 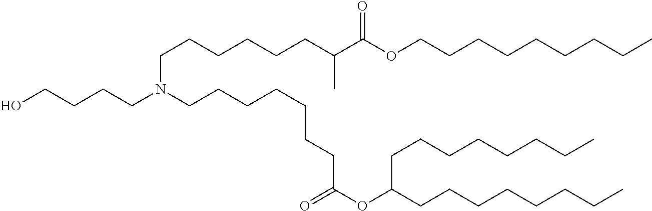 [SMILES: CCCCCCCCCOC(=O)C(C)CCCCCCN(CCCCO)CCCCCCCC(=O)OC(CCCCCCCC)CCCCCCCC] | Compound 62 |
| 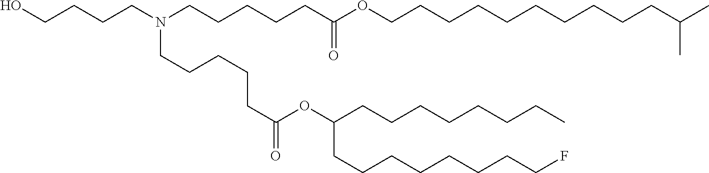 [SMILES: CCCCCCCCC(CCCCCCCCF)OC(=O)CCCCCN(CCCCO)CCCCCC(=O)OCCCCCCCCCCC(C)C] | Compound 63 |
| 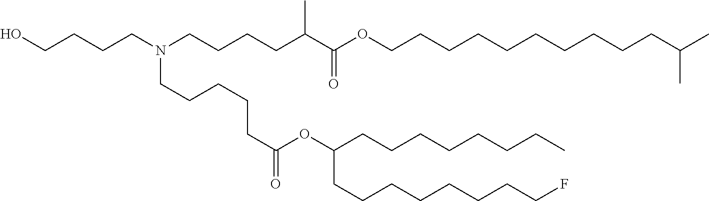 [SMILES: CCCCCCCCC(CCCCCCCCF)OC(=O)CCCCCN(CCCCO)CCCCC(C)C(=O)OCCCCCCCCCCC(C)C] | Compound 64 |
| 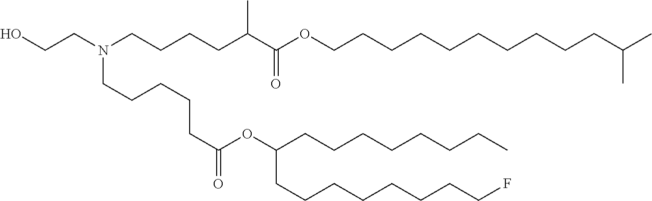 [SMILES: CCCCCCCCC(CCCCCCCCF)OC(=O)CCCCCN(CCO)CCCCC(C)C(=O)OCCCCCCCCCCC(C)C] | Compound 65 |
| 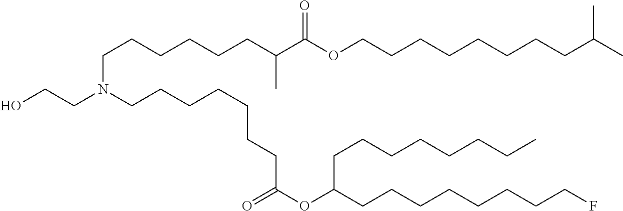 [SMILES: CCCCCCCCC(CCCCCCCCF)OC(=O)CCCCCCCN(CCO)CCCCCCC(C)C(=O)OCCCCCCCCC(C)C] | Compound 66 |
| 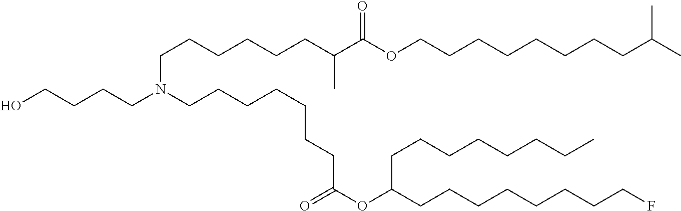 [SMILES: CCCCCCCCC(CCCCCCCCF)OC(=O)CCCCCCCN(CCCCO)CCCCCCC(C)C(=O)OCCCCCCCCC(C)C] | Compound 67 |
| 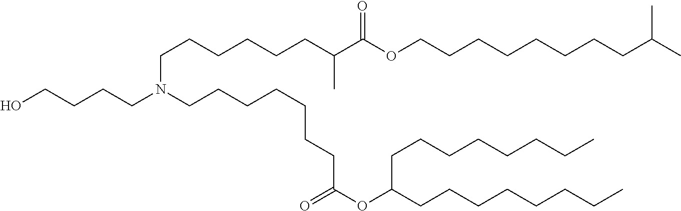 [SMILES: CCCCCCCCC(CCCCCCCC)OC(=O)CCCCCCCN(CCCCO)CCCCCCC(C)C(=O)OCCCCCCCCC(C)C] | Compound 68 |
| 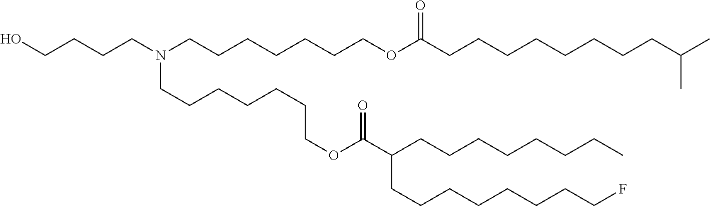 [SMILES: CCCCCCCCC(CCCCCCCCF)C(=O)OCCCCCCCN(CCCCO)CCCCCCCOC(=O)CCCCCCCCC(C)C] | Compound 69 |
| 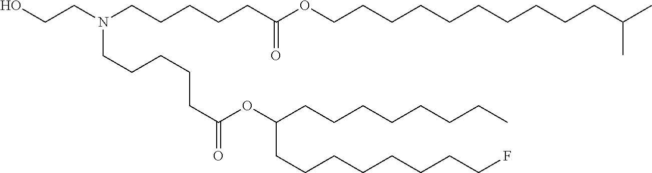 [SMILES: CCCCCCCCC(CCCCCCCCF)OC(=O)CCCCCN(CCO)CCCCCC(=O)OCCCCCCCCCCC(C)C] | Compound 70 |
| 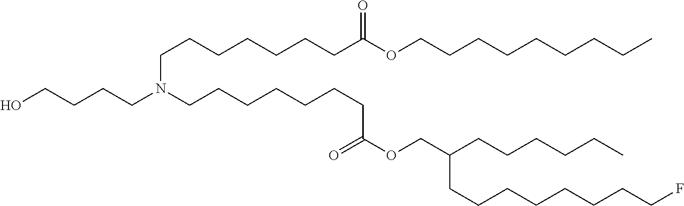 [SMILES: CCCCCCCCCOC(=O)CCCCCCCN(CCCCO)CCCCCCCC(=O)OCC(CCCCCC)CCCCCCCCF] | Compound 71 |
| 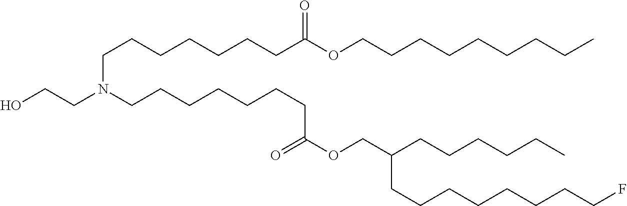 [SMILES: CCCCCCCCCOC(=O)CCCCCCCN(CCO)CCCCCCCC(=O)OCC(CCCCCC)CCCCCCCCF] | Compound 72 |
| 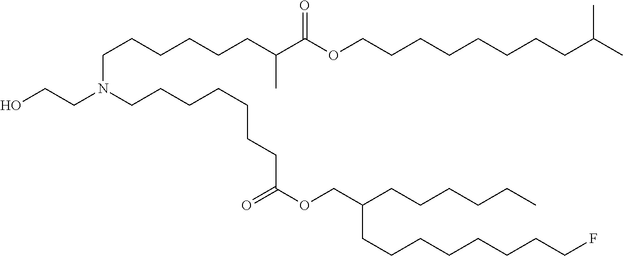 [SMILES: CCCCCCC(CCCCCCCCF)COC(=O)CCCCCCCN(CCO)CCCCCCC(C)C(=O)OCCCCCCCCC(C)C] | Compound 73 |
| 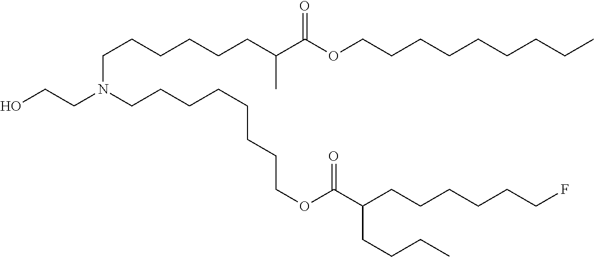 [SMILES: CCCCCCCCCOC(=O)C(C)CCCCCCN(CCO)CCCCCCCCOC(=O)C(CCCC)CCCCCCF] | Compound 74 |
| 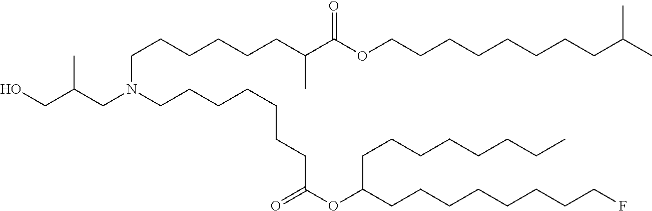 [SMILES: CCCCCCCCC(CCCCCCCCF)OC(=O)CCCCCCCN(CCCCCCC(C)C(=O)OCCCCCCCCC(C)C)CC(C)CO] | Compound 75 |
| 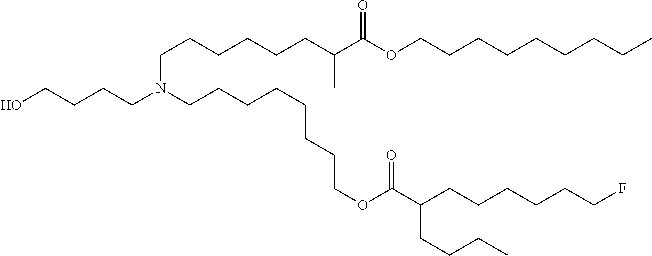 [SMILES: CCCCCCCCCOC(=O)C(C)CCCCCCN(CCCCO)CCCCCCCCOC(=O)C(CCCC)CCCCCCF] | Compound 76 |
| 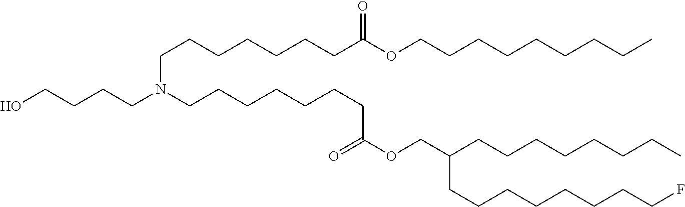 [SMILES: CCCCCCCCCOC(=O)CCCCCCCN(CCCCO)CCCCCCCC(=O)OCC(CCCCCCCC)CCCCCCCCF] | Compound 77 |
| 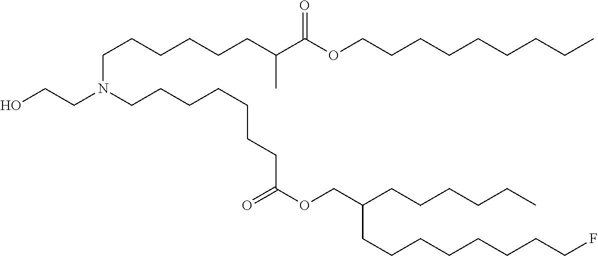 [SMILES: CCCCCCCCCOC(=O)C(C)CCCCCCN(CCO)CCCCCCCC(=O)OCC(CCCCCC)CCCCCCCCF] | Compound 78 |
| 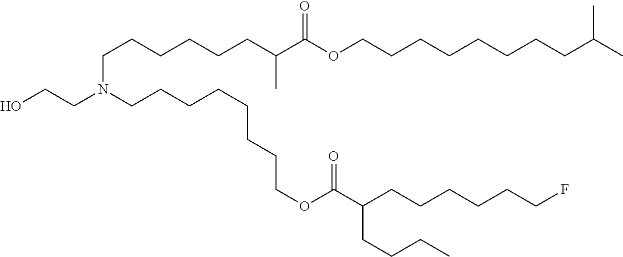 [SMILES: CCCCC(CCCCCCF)C(=O)OCCCCCCCCN(CCO)CCCCCCC(C)C(=O)OCCCCCCCCC(C)C] | Compound 79 |
| 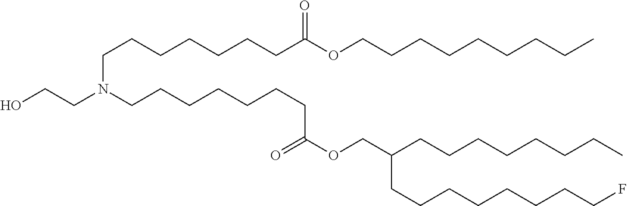 [SMILES: CCCCCCCCCOC(=O)CCCCCCCN(CCO)CCCCCCCC(=O)OCC(CCCCCCCC)CCCCCCCCF] | Compound 80 |
| 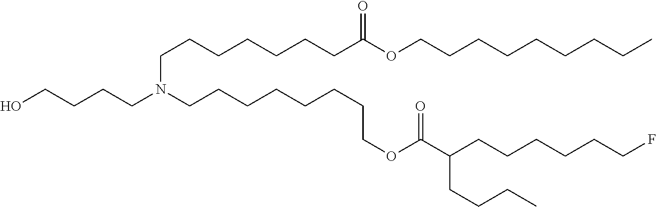 [SMILES: CCCCCCCCCOC(=O)CCCCCCCN(CCCCO)CCCCCCCCOC(=O)C(CCCC)CCCCCCF] | Compound 81 |
| 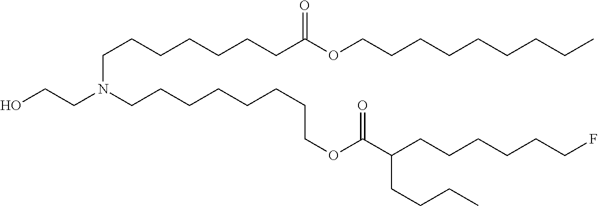 [SMILES: CCCCCCCCCOC(=O)CCCCCCCN(CCO)CCCCCCCCOC(=O)C(CCCC)CCCCCCF] | Compound 82 |
| 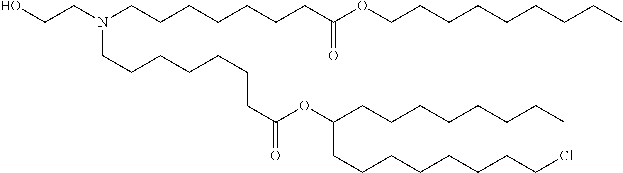 [SMILES: CCCCCCCCCOC(=O)CCCCCCCN(CCO)CCCCCCCC(=O)OC(CCCCCCCC)CCCCCCCCCl] | Compound 83 |
| 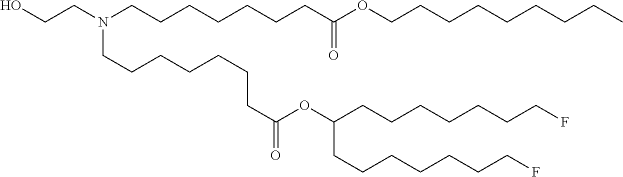 [SMILES: CCCCCCCCCOC(=O)CCCCCCCN(CCO)CCCCCCCC(=O)OC(CCCCCCCF)CCCCCCCF] | Compound 84 |
| 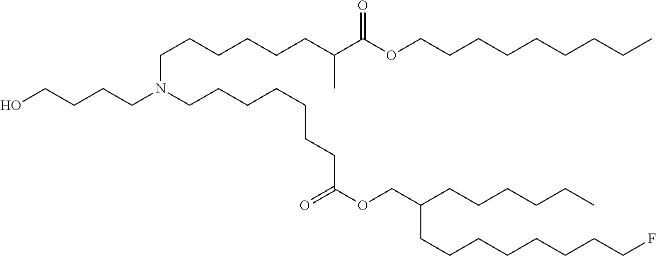 [SMILES: CCCCCCCCCOC(=O)C(C)CCCCCCN(CCCCO)CCCCCCCC(=O)OCC(CCCCCC)CCCCCCCCF] | Compound 85 |
| 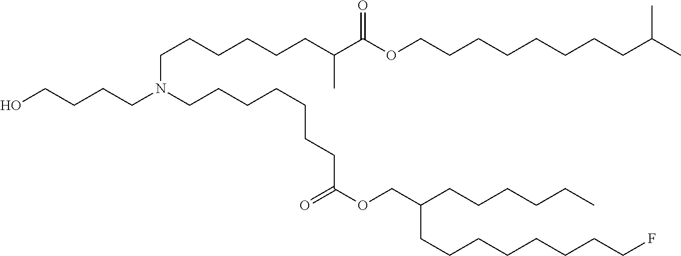 [SMILES: CCCCCCC(CCCCCCCCF)COC(=O)CCCCCCCN(CCCCO)CCCCCCC(C)C(=O)OCCCCCCCCC(C)C] | Compound 86 |
| 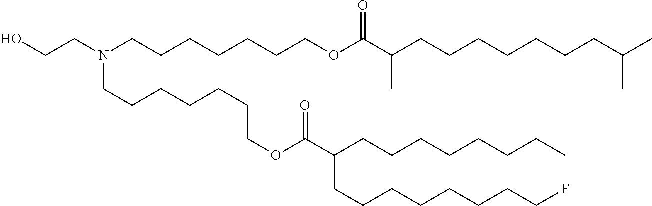 [SMILES: CCCCCCCCC(CCCCCCCCF)C(=O)OCCCCCCCN(CCO)CCCCCCCOC(=O)C(C)CCCCCCCC(C)C] | Compound 87 |
| 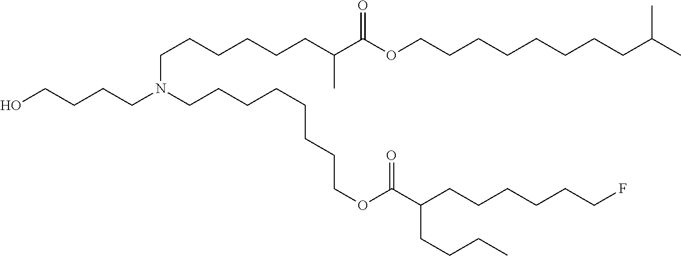 [SMILES: CCCCC(CCCCCCF)C(=O)OCCCCCCCCN(CCCCO)CCCCCCC(C)C(=O)OCCCCCCCCC(C)C] | Compound 88 |
| 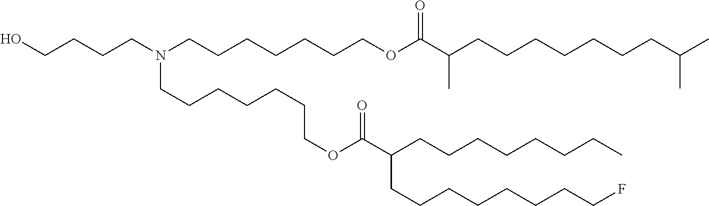 [SMILES: CCCCCCCCC(CCCCCCCCF)C(=O)OCCCCCCCN(CCCCO)CCCCCCCOC(=O)C(C)CCCCCCCC(C)C] | Compound 89 |

TABLE 3
Additional Exemplary Amino lipids

| Structure | Name |
|---|---|
| 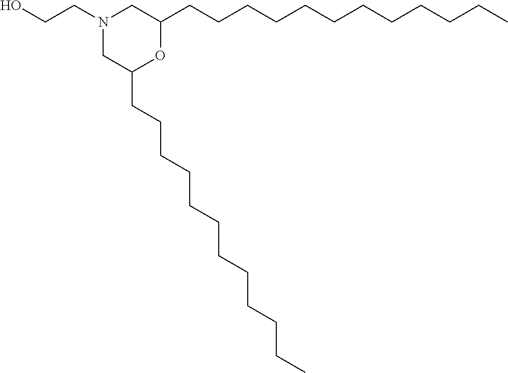 [SMILES: CCCCCCCCCCCCC1CN(CCO)CC(CCCCCCCCCCCC)O1] | Compound 90 |
| 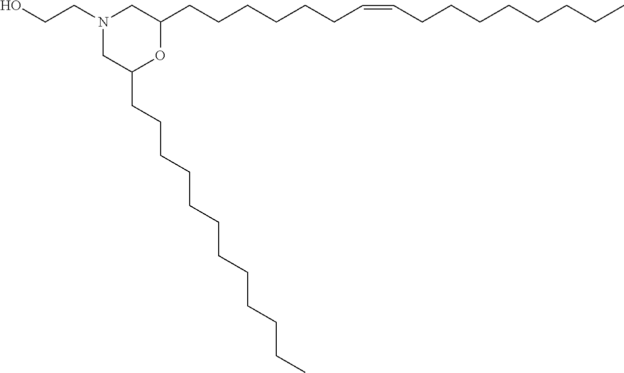 [SMILES: CCCCCCCC/C=C\CCCCCCC1CN(CCO)CC(CCCCCCCCCCCC)O1] | Compound 91 |
| 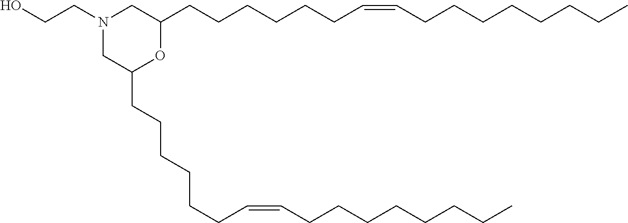 [SMILES: CCCCCCCC/C=C\CCCCCCC1CN(CCO)CC(CCCCCC/C=C\CCCCCCCC)O1] | Compound 92 |
| 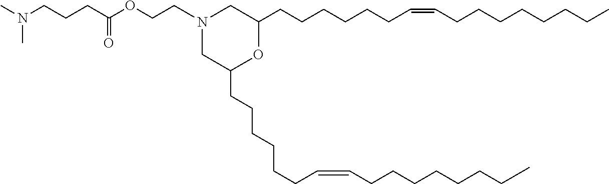 [SMILES: CCCCCCCC/C=C\CCCCCCC1CN(CCOC(=O)CCCN(C)C)CC(CCCCCC/C=C\CCCCCCCC)O1] | Compound 93 |
| 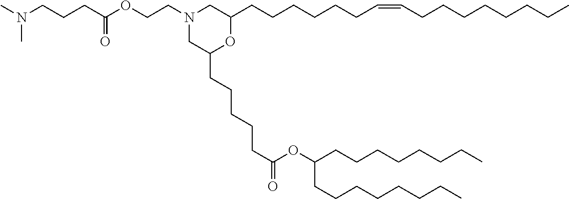 [SMILES: CCCCCCCC/C=C\CCCCCCC1CN(CCOC(=O)CCCN(C)C)CC(CCCCCC(=O)OC(CCCCCCCC)CCCCCCCC)O1] | Compound 94 |
| 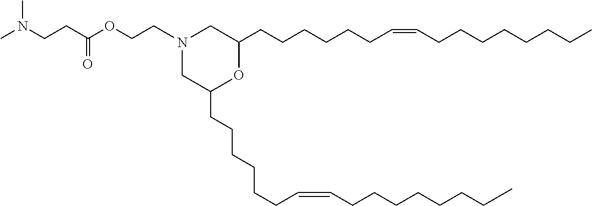 [SMILES: CCCCCCCC/C=C\CCCCCCC1CN(CCOC(=O)CCN(C)C)CC(CCCCCC/C=C\CCCCCCCC)O1] | Compound 95 |
| 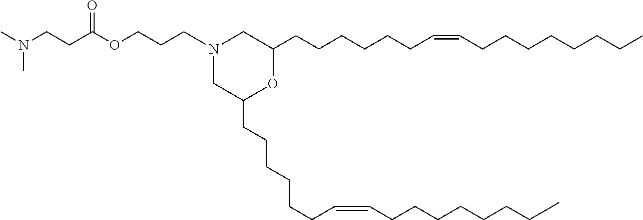 [SMILES: CCCCCCCC/C=C\CCCCCCC1CN(CCCOC(=O)CCN(C)C)CC(CCCCCC/C=C\CCCCCCCC)O1] | Compound 96 |
| 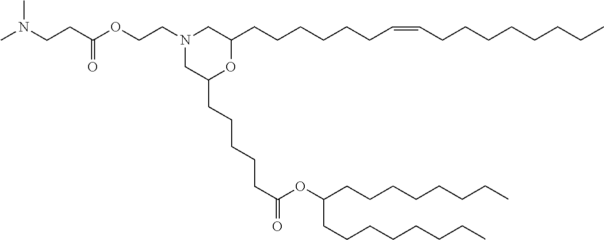 [SMILES: CCCCCCCC/C=C\CCCCCCC1CN(CCOC(=O)CCN(C)C)CC(CCCCCC(=O)OC(CCCCCCCC)CCCCCCCC)O1] | Compound 97 |
| 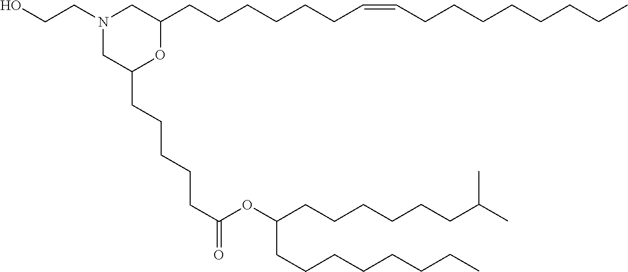 [SMILES: CCCCCCCC/C=C\CCCCCCC1CN(CCO)CC(CCCCCC(=O)OC(CCCCCCCC)CCCCCCC(C)C)O1] | Compound 98 |
| 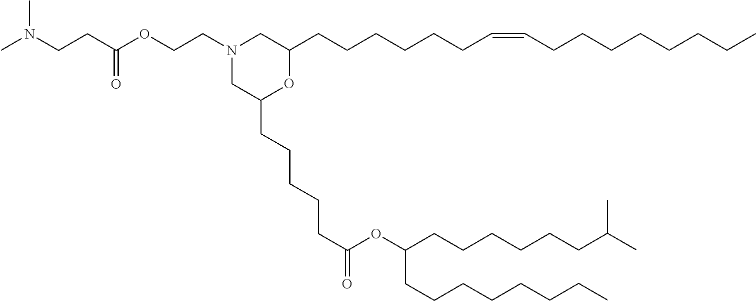 [SMILES: CCCCCCCC/C=C\CCCCCCC1CN(CCOC(=O)CCN(C)C)CC(CCCCCC(=O)OC(CCCCCCCC)CCCCCCC(C)C)O1] | Compound 99 |
| 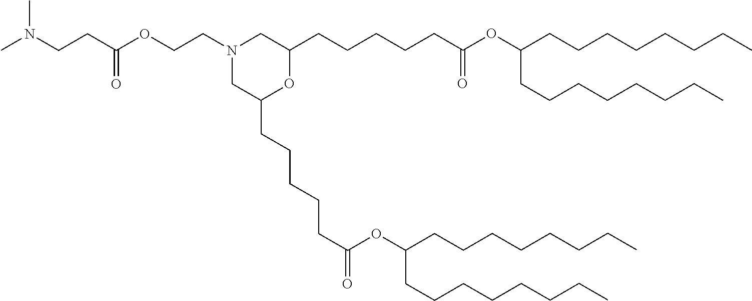 [SMILES: CCCCCCCCC(CCCCCCCC)OC(=O)CCCCCC1CN(CCOC(=O)CCN(C)C)CC(CCCCCC(=O)OC(CCCCCCCC)CCCCCCCC)O1] | Compound 100 |
| 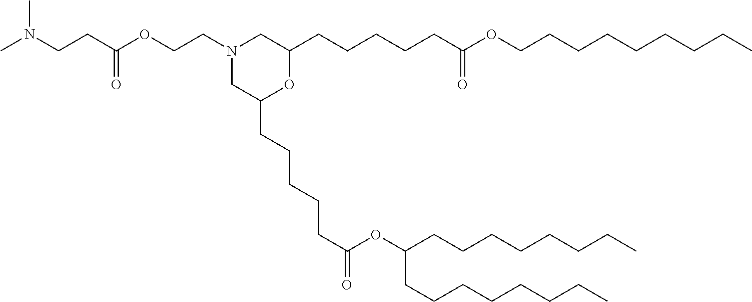 [SMILES: CCCCCCCCCOC(=O)CCCCCC1CN(CCOC(=O)CCN(C)C)CC(CCCCCC(=O)OC(CCCCCCCC)CCCCCCCC)O1] | Compound 101 |

In some embodiments, the LNP composition comprises an amino lipid. Exemplary amino lipids include, but are not limited to, the lipids in Table 1, Table 2, and Table 3. In some embodiments, the LNP composition comprises an amino lipid of having the structure of Formula I′, Formula I′-a, Formula I, Formula I-a, Formula I-b, Formula II, Formula III, Formula III-a, Formula IV, Formula IV-a, or Formula V, or a pharmaceutically acceptable salt thereof. In some embodiments, the LNP comprises a plurality of amino lipids. In some embodiments, the LNP composition comprises 2, 3, 4, 5, 6, 7, 8, 9, 10, or more amino lipids. In other embodiments, the LNP composition comprises at least 2, at least 3, at least 4, at least 5, at least 6, at least 7, at least 9, at least 10, or at least 20 amino lipids. In certain embodiments, the LNP composition comprises at most 2, at most 3, at most 4, at most 5, at most 6, at most 7, at most 9, at most 10, at most 20, or at most 30 amino lipids.

Additional Cationic and/or Ionizable Lipids

In some embodiments, the ionizable lipid comprises one or more ionizable nitrogen atoms. In some embodiments, at least one of the one or more ionizable nitrogen atoms is positively charged. In some embodiments, at least 10 mol %, 20 mol %, 30 mol %, 40 mol %, 50 mol %, 60 mol %, 70 mol %, 80 mol %, 90 mol %, 95 mol %, or 99 mol % of the ionizable nitrogen atoms in the LNP composition are positively charged. In some embodiments, the amino lipid comprises a primary amine, a secondary amine, a tertiary amine, an imine, an amide, a guanidine moiety, a histidine residue, a lysine residue, an arginine residue, or any combination thereof. In some embodiments, the amino lipid comprises a primary amine, a secondary amine, a tertiary amine, a guanidine moiety, or any combination thereof. In some embodiments, the amino lipid comprises a tertiary amine.

In some embodiments, the amino lipid is a cationic lipid. In some embodiments, the amino lipid is an ionizable lipid. In some embodiments, the amino lipid comprises one or more nitrogen atoms. In some embodiments, the amino lipid comprises one or more ionizable nitrogen atoms. Exemplary cationic and/or ionizable lipids include, but are not limited to, 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and (2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)).

### PEG-Lipid

In some embodiments, the described LNP composition comprises a PEG-lipid. In some embodiments, the described LNP composition comprises two or more PEG-lipids. Exemplary PEG-lipids include, but not limited to, PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, the one or more PEG-lipids can comprise PEG-c-DOMG, PEG-DMG, PEG-DLPE, PEG-DMPE, PEG-DPPC, a PEG-DSPE lipid, or a combination thereof. In some embodiments, PEG moiety is an optionally substituted linear or branched polymer of ethylene glycol or ethylene oxide. In some embodiments, the PEG moiety is substituted, e.g., by one or more alkyl, alkoxy, acyl, hydroxy, or aryl groups. In some embodiments, the PEG moiety includes PEG copolymer such as PEG-polyurethane or PEG-polypropylene (see, e.g., J. Milton Harris, Poly(ethylene glycol) chemistry: biotechnical and biomedical applications (1992)). In some embodiments, the PEG moiety does not include PEG copolymers, e.g., it may be a PEG monopolymer. Exemplary PEG-lipids include, but are not limited to, PEG-dilauroylglycerol, PEG-dimyristoylglycerol (PEG-DMG), PEG-dipalmitoylglycerol, PEG-distearoylgiycerol (PEG-DSPE), PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol, and PEG-DMB (3,4-Ditetradecoxylbenzyl-[omega]-methyl-poly(ethylene glycol) ether), 1,2-dimyristoyl-sn-glycero-3-phosphoethanolamine-N-[methoxy (polyethylene glycol)-2000]).

In some embodiments, the PEG-lipid is a PEG-lipid conjugate, for example, PEG coupled to dialkyloxypropyls (e.g., PEG-DAA conjugates), PEG coupled to diacylglycerols (e.g., PEG-DAG conjugates), PEG coupled to cholesterol, PEG coupled to phosphatidylethanolamines, and PEG conjugated to ceramides (see, e.g., U.S. Pat. No. 5,885,613), cationic PEG lipids, polyoxazoline (POZ)-lipid conjugates (e.g., POZ-DAA conjugates; see, e.g., WO 2010/006282), polyamide oligomers (e.g., ATTA-lipid conjugates), and mixtures thereof.

In some embodiments, the PEG lipid is a PEG-modified phosphatidy-lethanolamine, a PEG-modified phosphatidic acid, a PEG-modified ceramide, a PEG-modified dialkylamine, a PEG-modified diacylglycerol, or a PEG-modified dialkylglycerol.

In some embodiments, the PEG-lipid comprises one or more ethylene glycol units, for example, at least 1, at least 2, at least 5, at least 10, at least 20, at least 30, at least 40, at least 50, at least 60, at least 70, at least 80, at least 90, at least 100, at least 120, or at least 150 ethylene glycol units. In some embodiments, a number average molecular weight of the PEG-lipids is from about 200 Da to about 5000 Da. In some embodiments, a number average molecular weight of the PEG-lipids is from about 500 Da to about 3000 Da. In some embodiments, a number average molecular weight of the PEG-lipids is from about 750 Da to about 2500 Da. In some embodiments, a number average molecular weight of the PEG-lipids is from about 750 Da to about 2500 Da. In some embodiments, a number average molecular weight of the PEG-lipids is about 500 Da, about 750 Da, about 1000 Da, about 1250 Da, about 1500 Da, about 1750 Da, or about 2000 Da. In some embodiments, a polydispersity index (PDI) of the one or more PEG-lipids is smaller than 2. In some embodiments, a PDI of the one or more PEG-lipids is at most 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0. In some embodiments, a PDI of the one or more PEG-lipids is at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0.

In some embodiments, the PEG-lipid comprises from about 0.1 mol % to about 10 mol % of the total lipid present in the particle. In some embodiments, the PEG-lipid comprises from about 0.1 mol % to about 6 mol % of the total lipid present in the particle. In some embodiments, the PEG-lipid comprises from about 0.5 mol % to about 5 mol % of the total lipid present in the particle. In some embodiments, the PEG-lipid comprises from about 1 mol % to about 3 mol % of the total lipid present in the particle. In some embodiments, the PEG-lipid comprises about 2.0 mol % to about 2.5 mol % of the total lipid present in the particle. In some embodiments, the PEG-lipid comprises about 1 mol %, about 1.1 mol %, about 1.2 mol %, about 1.3 mol %, about 1.4 mol %, about 1.5 mol %, about 1.6 mol %, about 1.7 mol %, about 1.8 mol %, about 1.9 mol %, about 2.0 mol %, about 2.1 mol %, about 2.2 mol %, about 2.3 mol %, about 2.4 mol %, about 2.5 mol %, about 2.6 mol %, about 2.7 mol %, about 2.8 mol %, about 2.9 mol %, or about 3.0 mol % of the total lipid present in the particle.

In some embodiments, the LNP composition comprises a plurality of PEG-lipids, for example, at least 2, 3, 4, 5, 6, 7, 8, 9, 10, or more distinct PEG-lipids.

### Phospholipid

In some embodiments, the described LNP composition comprises a phospholipid. In some embodiments, the phospholipid comprises a lipid selected from the group consisting of: phosphatidylcholine (PC), phosphatidylethanolamine amine, glycerophospholipid, sphingophospholipids, Guriserohosuhono, sphingolipids phosphono lipids, natural lecithins, and hydrogenated phospholipid. In some embodiments, the phospholipid comprises a phosphatidylcholine. Exemplary phosphatidylcholines include, but are not limited to, soybean phosphatidylcholine, egg yolk phosphatidylcholine (EPC), distearoylphosphatidylcholine, 1,2-distearoyl-sn-glycero-3-phosphocholine (DSPC), dipalmitoyl phosphatidylcholine, dipalmitoyl-sn-glycero-3-phosphocholine (DPPC), palmitoyl oleoyl phosphatidylcholine (POPC), dimyristoyl phosphatidylcholine (DMPC), and dioleoyl phosphatidylcholine (DOPC). In certain specific embodiments, the phospholipid is DSPC.

In some embodiments, the phospholipid comprises a phosphatidylethanolamine amine. In some embodiments, the phosphatidylethanolamine amine is distearoyl phosphatidylethanolamine (DSPE), dipalmitoyl phosphatidyl ethanolamine (DPPE), dioleoyl phosphatidylethanolamine (DOPE), dimyristoyl phosphoethanolamine (DMPE), 16-0-Monome Le PE, 16-0-dimethyl PE, 18-1-trans PE, palmitoyl oleoyl-phosphatidylethanolamine (POPE), or 1-stearoyl-2-oleoyl-phosphatidyl ethanolamine (SOPE). In some embodiments, the phospholipid comprises a glycerophospholipid. In some embodiments, the glycerophospholipid is plasmalogen, phosphatidate, or phosphatidylcholine. In some embodiments, the glycerophospholipid is phosphatidylserine, phosphatidic acid, phosphatidylglycerol, phosphatidylinositol, palmitoyl oleoyl phosphatidylglycerol (POPG), or lysophosphatidylcholine. In some embodiments, the phospholipid comprises a sphingophospholipid. In some embodiments, the sphingophospholipid is sphingomyelin, ceramide phosphoethanolamine, ceramide phosphoglycerol, or ceramide phosphoglycerophosphoric acid. In some embodiments, the phospholipid comprises a natural lecithin. In some embodiments, the natural lecithin is egg yolk lecithin or soybean lecithin. In some embodiments, the phospholipid comprises a hydrogenated phospholipid. In some embodiments, the hydrogenated phospholipid is hydrogenated soybean phosphatidylcholine. In some embodiments, the phospholipid is selected from the group consisting of: phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine, and dilinoleoylphosphatidylcholine.

In some embodiments, the phospholipid comprises a lipid selected from: 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin.

In some embodiments, the phospholipid comprises a phospholipid moiety and one or more fatty acid moieties. In some embodiments, the phospholipid moiety comprises phosphatidyl choline, phosphatidyl ethanolamine, phosphatidyl glycerol, phosphatidyl serine, phosphatidic acid, 2-lysophosphatidyl choline, or a sphingomyelin. In some embodiments, a fatty acid moiety comprises lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, or docosahexaenoic acid. In some specific embodiments, a phospholipids functionalized with or cross-linked to one or more alkynes, which may undergo a copper-catalyzed cycloaddition upon exposure to an azide.

In some embodiments, the LNP composition comprises a plurality of phospholipids, for example, at least 2, 3, 4, 5, or more distinct phospholipids. In some embodiments, the phospholipid comprises from 1 mol % to 20 mol % of the total lipid present in the particle. In some embodiments, the phospholipid comprises from about 5 mol % to about 15 mol % of the total lipid present in the particle. In some embodiments, the phospholipid comprises from about 8 mol % to about 12 mol % of the total lipid present in the particle. In some embodiments, the phospholipid comprises from about 9 mol %, 10 mol %, or 11 mol % of the total lipid present in the particle.

### Structural Lipids

In some embodiments, the LNP composition comprises a structural lipid. In some embodiments, the structural lipid is a steroid, sterol, alkyl resoreinol, cholesterol or derivative thereof, fecosterol, sitosterol, ergosterol, campesterol, stigmasterol, brassicasterol, tomatidine, tomatine, ursolic acid, alpha-tocopherol, or a combination thereof. In some embodiments, the structural lipid is cholesterol, fecosterol, sitosterol, ergosterol, campesterol, stigmasterol, brassicasterol, tomatidine, ursolic acid, or alpha-tocopherol. In some embodiments, the structural lipid is a corticosteroid such as prednisolone, dexamethasone, prednisone, and hydrocortisone. In some embodiments, the cholesterol or derivative thereof is cholesterol, 5-heptadecylresorcinol, or cholesterol hemisuccinate. In some embodiments, the structural lipid is cholesterol.

In some embodiments, the structural lipid is a cholesterol derivative. In some embodiments, the cholesterol derivative is a polar cholesterol analogue. In some embodiments, the polar cholesterol analogue is 5α-cholestanol, 5β-coprostanol, cholesteryl-(2′-hydroxy)-ethyl ether, cholesteryl-(4′-hydroxy)-butyl ether, or 6-ketocholestanol. In some embodiments, the polar cholesterol analogue is cholesteryl-(4′-hydroxy)-butyl ether. In some embodiments, the cholesterol derivative is a non-polar cholesterol analogue. In some embodiments, the non-polar cholesterol analogue is 5α-cholestane, cholestenone, 5α-cholestanone, 5β-cholestanone, or cholesteryl decanoate.

In some embodiments, the cholesterol or the derivative thereof comprises from 20 mol % to 50 mol % of the total lipid present in the particle. In some embodiments, the cholesterol or the derivative thereof comprises about 20 mol %, about 21 mol %, about 22 mol %, about 23 mol %, about 24 mol %, about 25 mol %, about 26 mol %, about 27 mol %, about 28 mol %, about 29 mol %, about 30 mol %, about 31 mol %, about 32 mol %, about 33 mol %, about 34 mol %, about 35 mol %, about 36 mol %, about 37 mol %, about 38 mol %, about 39 mol %, about 40 mol %, about 41 mol %, about 42 mol %, about 43 mol %, about 44 mol %, about 45 mol %, about 46 mol %, about 47 mol %, about 48 mol %, or about 50 mol % of the total lipid present in the particle.

### Antioxidants

In some embodiments, the LNP described herein comprises one or more antioxidants. In some embodiments, the one or more antioxidants function to reduce a degradation of the cationic lipids, the payload, or both. In some embodiments, the one or more antioxidants comprise a hydrophilic antioxidant. In some embodiments, the one or more antioxidants is a chelating agent such as ethylenediaminetetraacetic acid (EDTA) and citrate. In some embodiments, the one or more antioxidants is EDTA. In some embodiments, the one or more antioxidants comprise a lipophilic antioxidant. In some embodiments, the lipophilic antioxidant comprises a vitamin E isomer or a polyphenol. In some embodiments, the one or more antioxidants are present in the LNP composition at a concentration of at least 1 mM, at least 10 mM, at least 20 mM, at least 50 mM, or at least 100 mM. In some embodiments, the one or more antioxidants are present in the particle at a concentration of about 20 mM.

### Payload

The nanoparticle compositions or LNPs described herein can be designed to deliver a payload such as a therapeutic agent. Exemplary therapeutic agents include, but are not limited to, antibodies (e.g., monoclonal, chimeric, humanized, nanobodies, and fragments thereof etc.), cholesterol, hormones, peptides, proteins, chemotherapeutics and other types of antineoplastic agents, low molecular weight drugs, vitamins, co-factors, nucleosides, nucleotides, oligonucleotides, enzymatic nucleic acids, antisense nucleic acids, triplex forming oligonucleotides, antisense DNA or RNA compositions, chimeric DNA: RNA compositions, allozymes, aptamers, ribozyme, decoys and analogs thereof, plasmids and other types of expression vectors, and small nucleic acid molecules, RNAi agents, short interfering nucleic acid (siNA), messenger ribonucleic acid (messenger RNA, mRNA), short interfering RNA (siRNA), double-stranded RNA (dsRNA), micro-RNA (miRNA), and short hairpin RNA (shRNA) molecules, peptide nucleic acid (PNA), a locked nucleic acid ribonucleotide (LNA), morpholino nucleotide, threose nucleic acid (TNA), glycol nucleic acid (GNA), sisiRNA (small internally segmented interfering RNA), aiRNA (asymmetrical interfering RNA), and siRNA with 1, 2 or more mismatches between the sense and anti-sense strand to relevant cells and/or tissues, such as in a cell culture, subject or organism. Therapeutic agents can be purified or partially purified, and can be naturally occurring or synthetic, or chemically modified. In some embodiments, the therapeutic agent is an RNAi agent, short interfering nucleic acid (siNA), short interfering RNA (siRNA), double-stranded RNA (dsRNA), micro-RNA (miRNA), or a short hairpin RNA (shRNA) molecule. In some embodiments, the therapeutic agent is an mRNA.

In some embodiments, the payload comprises one or more nucleic acid(s) (i.e., one or more nucleic acid molecular entities). In some embodiments, the nucleic acid is a single-stranded nucleic acid. In some embodiments, single-stranded nucleic acid is a DNA. In some embodiments, single-stranded nucleic acid is an RNA. In some embodiments, the nucleic acid is a double-stranded nucleic acid. In some embodiments, the double-stranded nucleic acid is a DNA. In some embodiments, the double-stranded nucleic acid is an RNA. In some embodiments, the double-stranded nucleic acid is a DNA-RNA hybrid. In some embodiments, the nucleic acid is a messenger RNA (mRNA), a microRNA, an asymmetrical interfering RNA (aiRNA), a small hairpin RNA (shRNA), or a Dicer-Substrate dsRNA. In some embodiments, the nucleic acid comprises a promoter. In some embodiments, the nucleic acid comprises an oncoselective motif, where the oncoselective motif increases expression of the nucleic acid in a cell associated with a disease or condition (e.g., cancer) compared to expression of the nucleic acid in a normal cell.

In some embodiments, the payload, such as one or more RNAs, are fully encapsulated within the lipid portion of the particle, thereby protecting the RNAs from nuclease degradation. Fully encapsulated can indicate that the RNA in the nucleic acid-lipid particle is not significantly degraded after exposure to serum or a nuclease assay that would significantly degrade free DNA or RNA. In some embodiments, the nucleic acid-lipid particle composition comprises a RNA molecule that is fully encapsulated within the lipid portion of the particles, such that from about 30% to about 100%, from about 40% to about 100%, from about 50% to about 100%, from about 60% to about 100%, from about 70% to about 100%, from about 80% to about 100%, from about 90% to about 100%, from about 30% to about 95%, from about 40% to about 95%, from about 50% to about 95%, from about 60% to about 95%, from about 70% to about 95%, from about 80% to about 95%, from about 85% to about 95%, from about 90% to about 95%, from about 30% to about 90%, from about 40% to about 90%, from about 50% to about 90%, from about 60% to about 90%, from about 70% to about 90%, from about 80% to about 90%, or at least about 30%, 35%, 40%, 45%, 50%, 55%, 60%, 65%, 70%, 75%, 80%, 85%, 90%, 91%, 92%, 93%, 94%, 95%, 96%, 97%, 98%, or 99% (or any fraction thereof or range therein) of the particles have the RNA encapsulated therein.

### Other Lipids

In some embodiments, the disclosed nanoparticles or LNP compositions comprise a helper lipid. In some embodiments, the disclosed LNP compositions comprise a neutral lipid. In some embodiments, the disclosed LNP compositions comprise a stealth lipid. In some embodiments, the disclosed LNP compositions comprises additional lipids.

As used herein, “neutral lipids” suitable for use in a lipid composition of the disclosure include, for example, a variety of neutral, uncharged or zwitterionic lipids. Examples of neutral phospholipids suitable for use in the present disclosure include, but are not limited to, 5-heptadecylbenzene-1,3-diol (resorcinol), dipalmitoylphosphatidylcholine (DPPC), distearoylphosphatidylcholine (DSPC), 1,2-Dioleoyl-sn-glycero-3-phosphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine and combinations thereof. In some embodiments, the neutral phospholipid may be selected from the group consisting of SPC and dimyristoyl phosphatidyl ethanolamine (DMPE). In some embodiments, the neutral phospholipid is DSPC. Neutral lipids can function to stabilize and improve processing of the LNPs.

“Helper lipids” can refer to lipids that enhance transfection (e.g. transfection of the nanoparticle including the biologically active agent). The mechanism by which the helper lipid enhances transfection includes enhancing particle stability. In some embodiments, the helper lipid enhances membrane fusogenicity. Helper lipids can include steroids, sterols, and alkyl resorcinols. Helper lipids suitable for use in the present disclosure can include, but are not limited to, cholesterol, 5-heptadecylresorcinol, and cholesterol hemisuccinate. In some embodiments, the helper lipid is cholesterol. In some embodiments, the helper lipid comprises cholesterol hemisuccinate.

“Stealth lipids” can refer to lipids that alter the length of time the nanoparticles can exist in vivo {e.g., in the blood). Stealth lipids can assist in the formulation process by, for example, reducing particle aggregation and controlling particle size. Stealth lipids used herein may modulate pharmacokinetic properties of the LNP. Stealth lipids suitable for use in a lipid composition of the disclosure can include, but are not limited to, stealth lipids having a hydrophilic head group linked to a lipid moiety. Stealth lipids suitable for use in a lipid composition of the present disclosure and information about the biochemistry of such lipids can be found in Romberg et al, Pharmaceutical Research, Vol. 25, No. 1, 2008, pg. 55-71 and Hoekstra et al, Biochimica et Biophysica Acta 1660 (2004) 41-52. Additional suitable PEG lipids are disclosed, e.g., in WO 2006/007712.

In some embodiments, the stealth lipid is a PEG-lipid. In one embodiment, the hydrophilic head group of stealth lipid comprises a polymer moiety selected from polymers based on PEG (sometimes referred to as poly(ethylene oxide)), poly(oxazoline), poly(vinyl alcohol), poly(glycerol), poly(N-vinylpyrrolidone), polyaminoacids and poly N-(2-hydroxypropyl)methacrylamide]. Stealth lipids can comprise a lipid moiety. In some embodiments, the lipid moiety of the stealth lipid may be derived from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. The dialkylglycerol or dialkylglycamide group can further comprise one or more substituted alkyl groups.

The structures and properties of helper lipids, neutral lipids, stealth lipids, and/or other lipids are further described in WO2017173054A1, WO2019067999A1, US20180290965A1, US20180147298A1, US20160375134A1, U.S. Pat. Nos. 8,236,770, 8,021,686, 8,236,770B2, U.S. Pat. No. 7,371,404B2, U.S. Pat. No. 7,780,983B2, U.S. Pat. No. 7,858,117B2, US20180200186A1, US20070087045A1, WO2018119514A1, and WO2019067992A1, all of which are hereby incorporated by reference in their entirety.

### LNP Formulations

The LNPs described herein can be designed for one or more specific applications or targets. The elements of a nanoparticle composition can be selected based on a particular application or target, and/or based on the efficacy, toxicity, expense, ease of use, and availability. Similarly, the particular formulation of a nanoparticle composition may be selected for the particular application or target.

In some embodiments, the described LNP formulations are designed for one or more specific applications or targets. For example, a nanoparticle composition may be designed to deliver a therapeutic agent such as an RNA to a particular cell, tissue, organ, or system or group thereof in a mammal's body. Physiochemical properties of nanoparticle compositions may be altered in order to increase selectivity for particular bodily targets. For instance, particle sizes may be adjusted based on the fenestration sizes of different organs. The therapeutic agent included in a nanoparticle composition may also be selected based on the desired delivery target or targets. For example, a therapeutic agent may be selected for a particular indication, condition, disease, or disorder and/or for delivery to a particular cell, tissue, organ, or system or group thereof (e.g., localized or specific delivery). In certain embodiments, a nanoparticle composition may include an mRNA encoding a polypeptide of interest capable of being translated within a cell to produce the polypeptide of interest. Such a composition may be designed to be specifically delivered to a particular organ.

The amount of a therapeutic agent in an LNP composition depends on the size, composition, desired target and/or application, or other properties of the nanoparticle composition. For example, the amount of an RNA comprised in a nanoparticle composition depends on the size, sequence, and other characteristics of the RNA. In some embodiments, the wt/wt ratio of the lipid component to a therapeutic agent in a nanoparticle composition is from about 5:1 to about 60:1, such as about 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. In some embodiments, the wt/wt ratio of the lipid component to a therapeutic agent is from about 10:1 to about 40:1. In certain embodiments, the wt/wt ratio is about 20:1. In some instances, the amount of a therapeutic agent in a nanoparticle composition is measured using absorption spectroscopy (e.g., ultraviolet-visible spectroscopy).

In some embodiments, an LNP composition comprises one or more nucleic acids such as RNAs. In some embodiments, the one or more RNAs, lipids, and amounts thereof may be selected to provide a specific N/P ratio. The N/P ratio can be selected from about 1 to about 30. The N/P ratio can be selected from about 2 to about 10. In some embodiments, the N/P ratio is from about 0.1 to about 50. In some embodiments, the N/P ratio is from about 2 to about 8. In some embodiments, the N/P ratio is from about 2 to about 15, from about 2 to about 10, from about 2 to about 8, from about 2 to about 6, from about 3 to about 15, from about 3 to about 10, from about 3 to about 8, from about 3 to about 6, from about 4 to about 15, from about 4 to about 10, from about 4 to about 8, or from about 4 to about 6. In some embodiments, the N/P ratio is from about 2 to about 8. In some embodiments, the N/P ratio is about 2, about 2.5, about 3, about 3.5, about 4, about 4.5, about 5, about 5.5, about 6, or about 6.5. In some embodiments, the N/P ratio is from about 4 to about 6. In some embodiments, the N/P ratio is about 4, about 4.5, about 5, about 5.5, or about 6. In some embodiments, the N/P ratio is from about 6 to about 8. In some embodiments, the N/P ratio is about 6.5, about 7, about 7.5, or about 8.

In some embodiments, the LNPs are formed with an average encapsulation efficiency ranging from about 50% to about 70%, from about 70% to about 90%, or from about 90% to about 100%. In some embodiments, the LNPs are formed with an average encapsulation efficiency ranging from about 75% to about 95%. In some embodiments, a nucleic acid (e.g., RNA) entrapment efficiency of a nanoparticle described herein is at least 80%, at least 85%, at least 90%, at least 91%, at least 92%, at least 93%, at least 94%, at least 95%, at least 96%, at least 97%, at least 98%, or at least 99%. In some embodiments, a nucleic acid entrapment efficiency of a nanoparticle described herein is from about 80% to about 100%, from about 85% to about 100%, from about 90% to about 100%, from about 95% to about 100%, from about 90% to about 99%, or from about 95% to about 99%. In some embodiments, a nucleic acid entrapment efficiency of a nanoparticle described herein is from about 90% to about 99%.

In some embodiments, nanoparticles described herein have a median diameter of about 10 nm to about 500 nm. In some embodiments, the median diameter of the nanoparticles described herein is from about 50 nm to about 150 nm, from about 60 nm to about 140 nm, from about 70 nm to about 130 nm, from about 80 nm to about 120 nm, or from about 90 nm to about 110 nm. In some embodiments, the median diameter of the nanoparticles described herein is about 60 nm, about 65 nm, about 70 nm, about 75 nm, about 80 nm, about 85 nm, about 90 nm, about 95 nm, about 100 nm, about 105 nm, about 110 nm, about 115 nm, or about 120 nm. Particle size and particle size distribution of the nanoparticles can be measured by light scattering using, for example, a Zetasizer Ultra ZSU 5700 (Malvern, USA). In some embodiments, the particle size distribution is unimodal.

### Pharmaceutical Compositions

In one aspect, disclosed herein are pharmaceutical compositions comprising one or more described compounds, nanoparticle compositions, or LNPs described herein. For example, a pharmaceutical composition can include one or more LNP compositions including one or more different payloads. In some embodiments, the payload comprises a nuclei acid described herein. In some embodiments, the payload comprises an RNA described herein. In some embodiments, the payload comprises an mRNA or a population of mRNA, where the population of mRNA encodes the same or different polypeptides. For example, the LNP can encapsulate a population of mRNA encoding at least one interleukin and at least one interferon. In some embodiments, the payload comprises at least one oncoselective motif or oncoselective element for selectively increasing expression of the payload in a cancer cell.

Pharmaceutical compositions can further include one or more pharmaceutically acceptable excipients, carrier, or accessory ingredients such as those described herein. General guidelines for the formulation and manufacture of pharmaceutical compositions and agents are available. Excipients or carriers can include any ingredient other than the compound(s) of the disclosure, the other lipid component(s) and the payload. An excipient may impart either a functional (e.g. drug release rate controlling) and/or a nonfunctional (e.g. processing aid or diluent) characteristic to the formulations. The choice of excipient and carrier can depend on factors such as the particular mode of administration, the effect of the excipient on solubility and stability, and the nature of the dosage form. Parenteral formulations are typically aqueous or oily solutions or suspensions. Excipients or carrier such as sugars (including but not restricted to glucose, mannitol, sorbitol, etc.), salts, carbohydrates and buffering agents (preferably to a pH of from 3 to 9) can be used. In some embodiments, the LNP compositions can be formulated with a sterile non-aqueous solution or as a dried form to be used in conjunction with a suitable vehicle such as sterile, pyrogen-free water (WFI).

In some embodiments, the excipient or carrier can make up greater than 50% of the total mass or volume of a pharmaceutical composition comprising a nanoparticle composition. For example, the excipient or carrier can make up 50%, 60%, 70%, 80%, 90%, or more of a pharmaceutical composition. In some embodiments, a pharmaceutically acceptable excipient or carrier is at least 95%, at least 96%, at least 97%, at least 98%, at least 99%, or 100% pure. In some embodiments, a pharmaceutical composition can comprise between 0.1% and 100% (wt/wt) of one or more nanoparticle compositions. In certain embodiments, the nanoparticle compositions and/or pharmaceutical compositions are refrigerated or frozen for storage and/or shipment (e.g., being stored at a temperature of 4° C. or lower, such as a temperature between about −150° C. and about 0° C. or between about −80° C. and about −20° C. In some embodiments, the nanoparticle compositions and/or pharmaceutical compositions are refrigerated or frozen at about −5° C., −10° C., −15° C., −20° C., −25° C., −30° C., −40° C., −50° C., −60° C., −70° C., −80° C., −90° C., −130° C., or −150° C.

The described LNP compositions and/or pharmaceutical compositions can be administered to any patient or subject, including those patients or subjects that may benefit from a therapeutic effect provided by the delivery of the payload to one or more particular cells, tissues, organs, or systems or groups thereof. In some embodiments, the subject is a mammal such as human. In some embodiments, the subject is non-human primates or mammals, including commercially relevant mammals such as cattle, pigs, hoses, sheep, cats, dogs, mice, and/or rats.

A pharmaceutical composition including one or more nanoparticle compositions may be prepared by any method known or hereafter developed in the art of pharmacology. In general, such preparatory methods include bringing the active ingredient into association with an excipient and/or one or more other accessory ingredients, and then, if desirable or necessary, dividing, shaping, and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient (e.g., nanoparticle composition). The amount of the active ingredient is generally equal to the dosage of the active ingredient which would be administered to a subject and/or a convenient fraction of such a dosage such as, for example, one-half or one-third of such a dosage. Pharmaceutical compositions may be prepared in a variety of forms suitable for a variety of routes and methods of administration. For example, pharmaceutical compositions may be prepared in liquid dosage forms (e.g., emulsions, microemulsions, nanoemulsions, solutions, suspensions, syrups, and elixirs), injectable forms, solid dosage forms (e.g., capsules, tablets, pills, powders, and granules), dosage forms for topical and/or transdermal administration (e.g., ointments, pastes, creams, lotions, gels, powders, solutions, sprays, inhalants, and patches), suspensions, powders, and other forms.

### Methods

The compounds, nanoparticle compositions (e.g., nanoparticle composition comprising a compound described herein), LNP compositions (e.g., LNP comprising a compound described herein), or pharmaceutical compositions disclosed herein can be used in methods modulating gene expression in a cell, both in vivo and in vitro. In some embodiments, the methods comprise contacting a cell with an LNP composition or a pharmaceutical composition described herein. In some embodiments, the cell is a mammalian cell. In some embodiments, the cell is a human cell. In some embodiments, the method comprises contacting a population of cells.

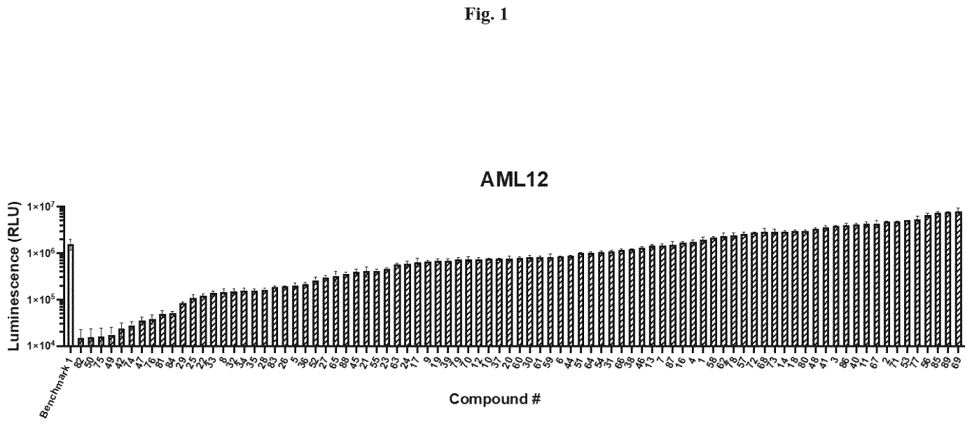

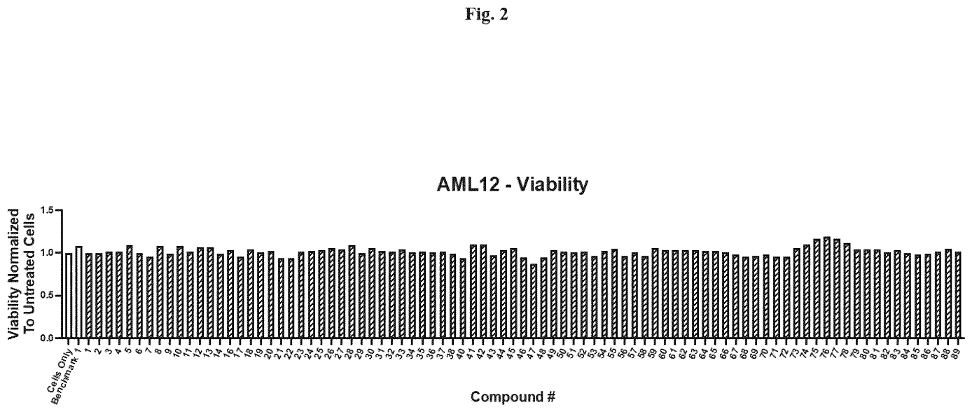

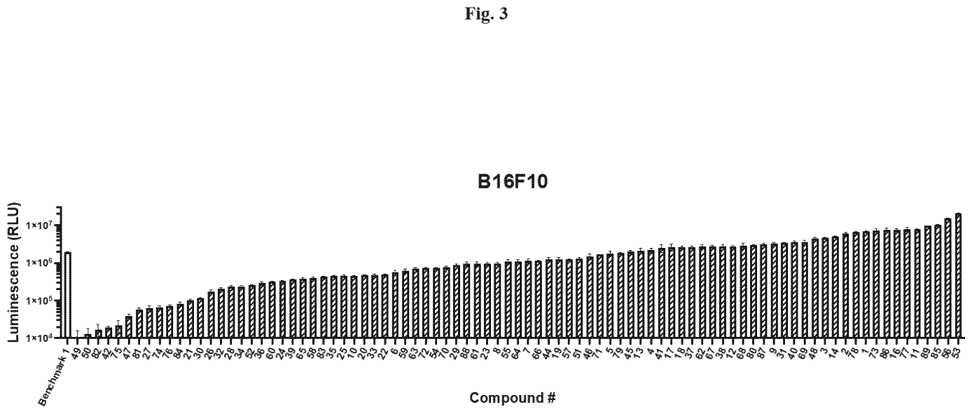

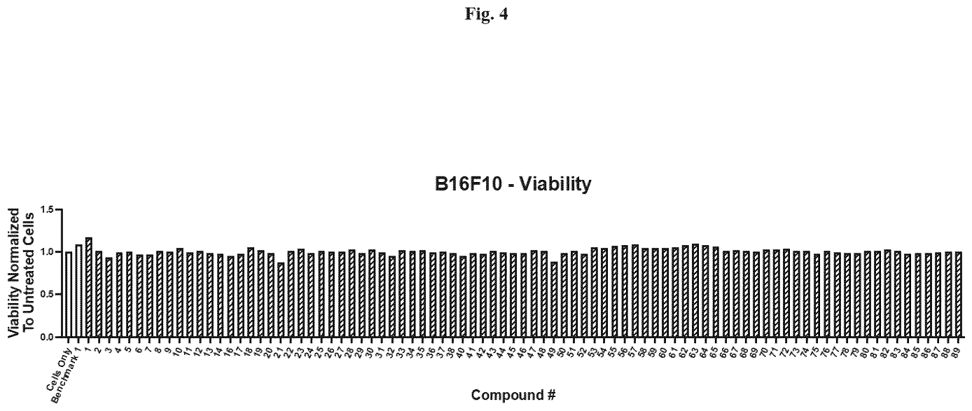

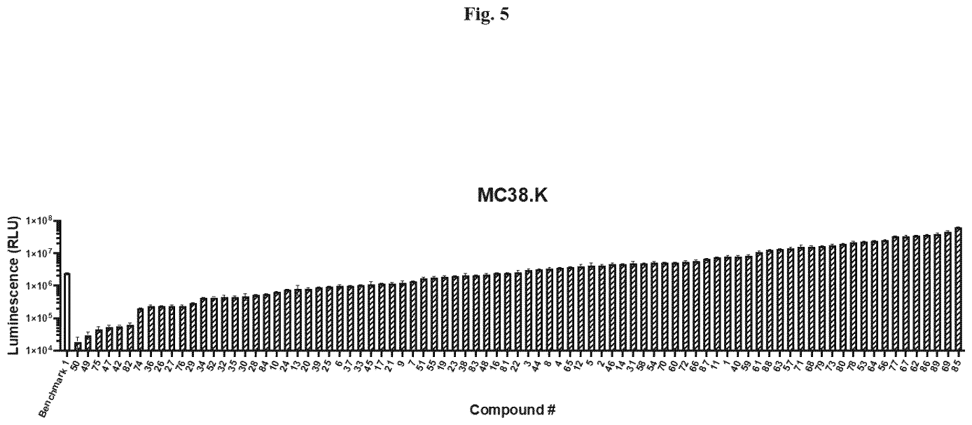

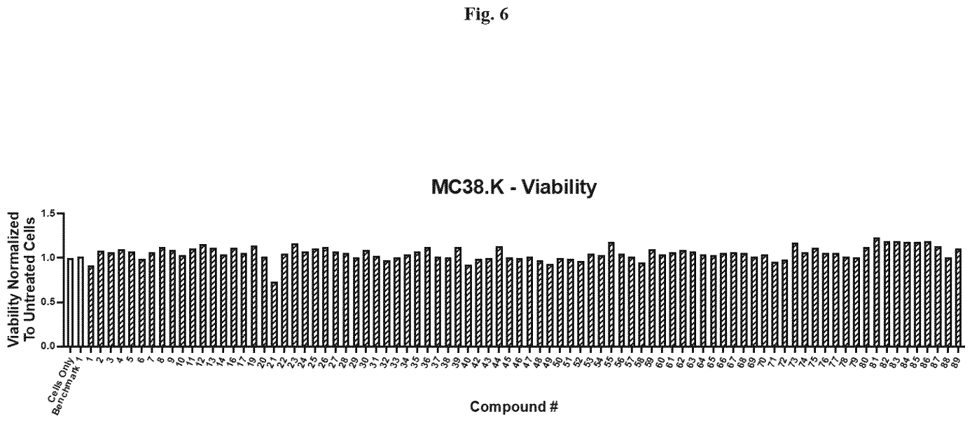

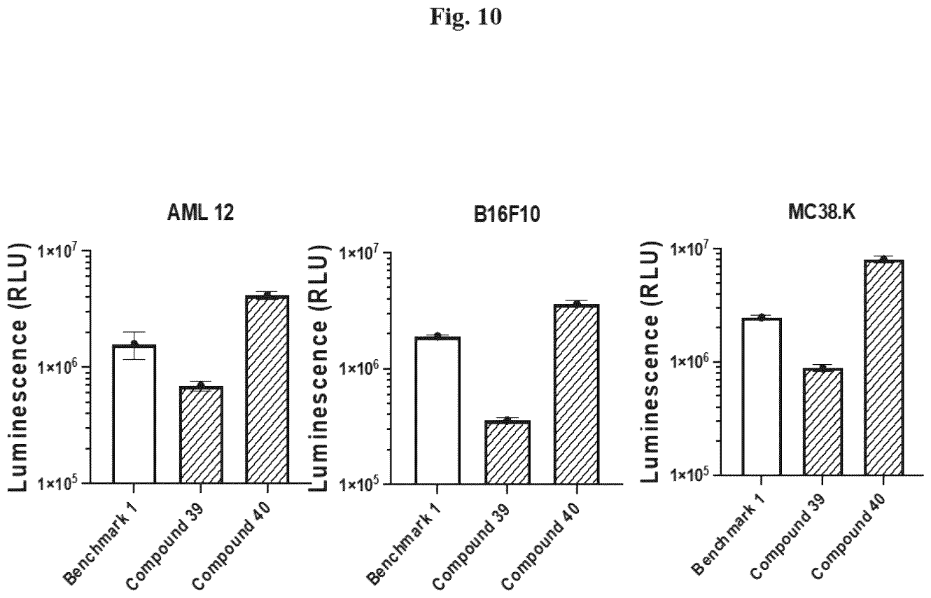

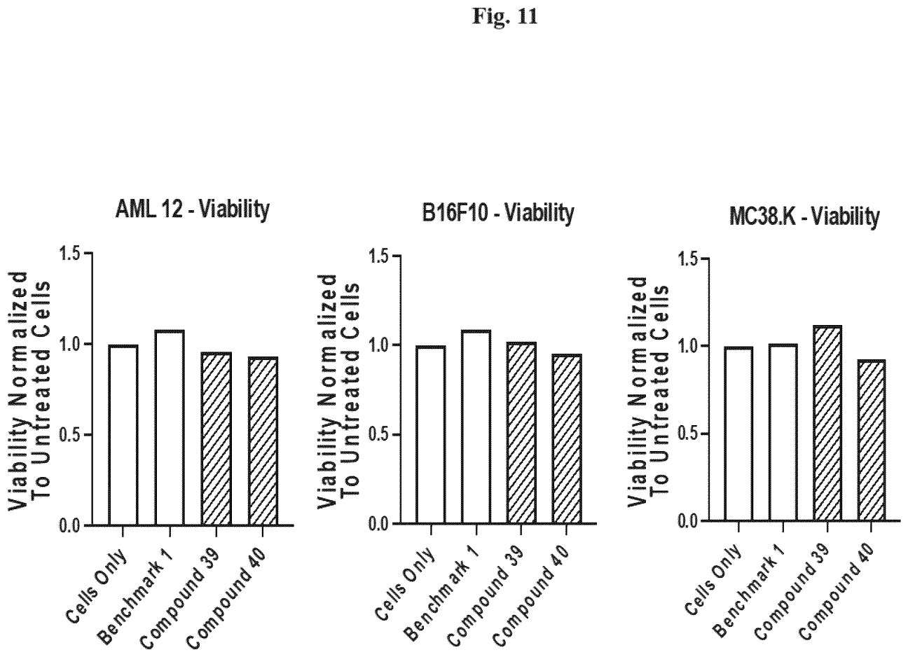

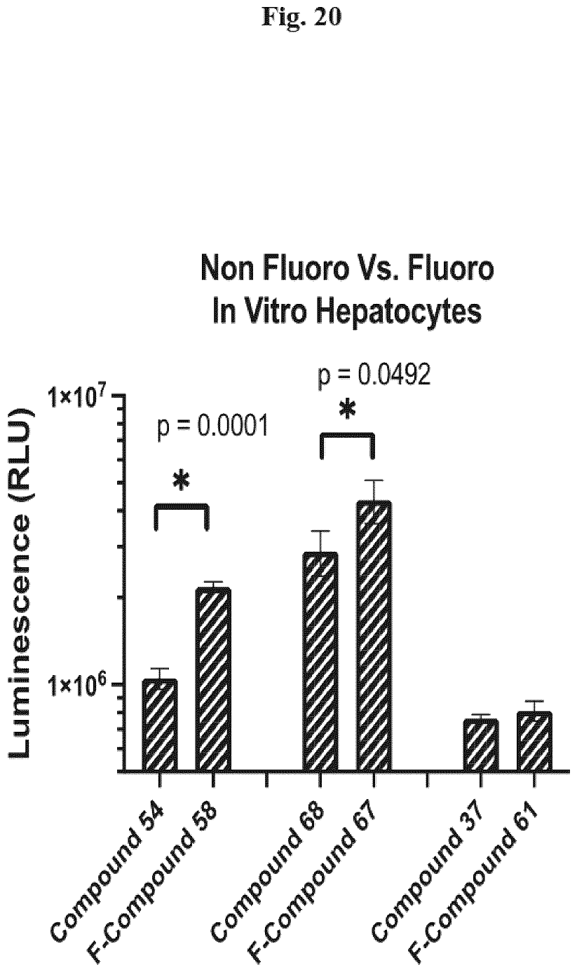

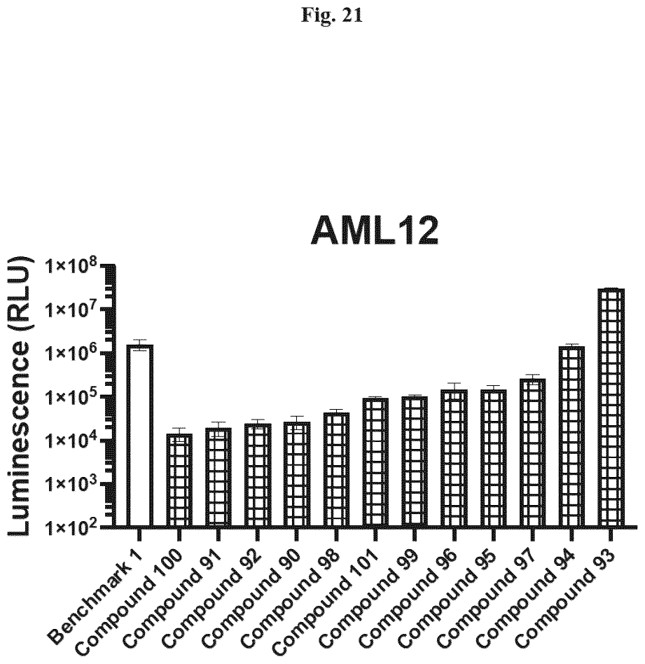

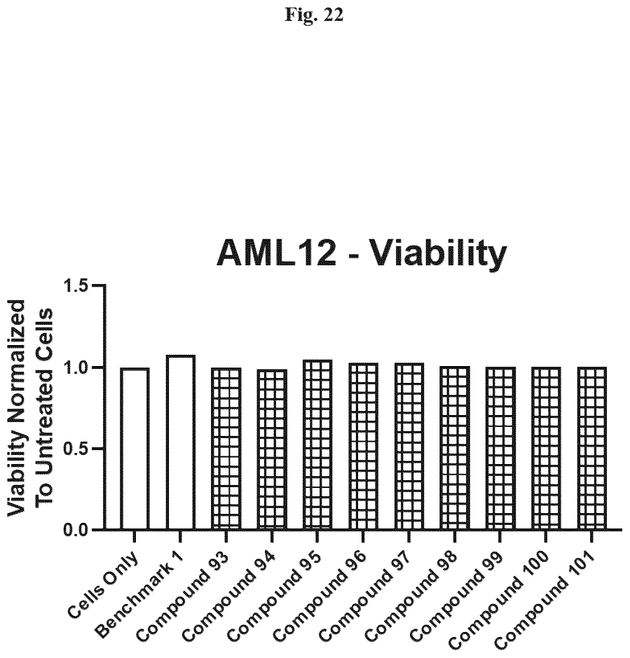

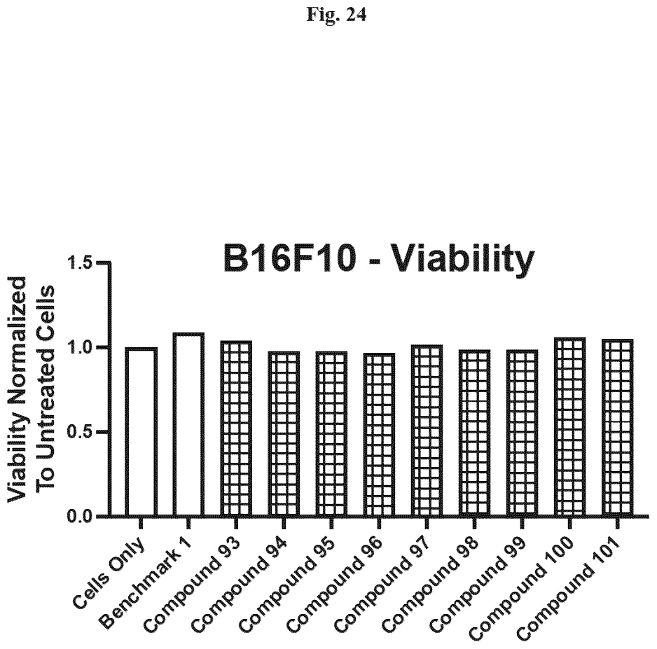

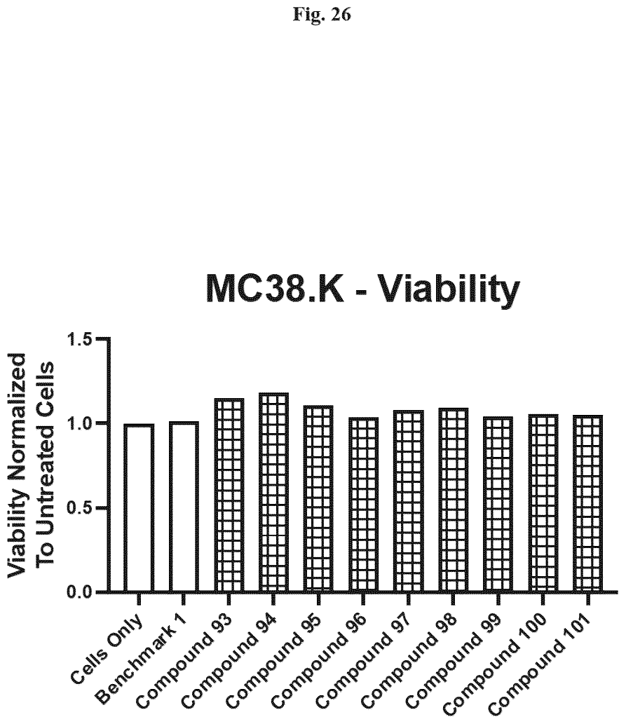

In some embodiments, an LNP comprising a compound described herein can increase transfection efficiency of a cell as shown in e.g., FIG. 1, FIG. 3, FIG. 5, FIG. 10, FIG. 20, and FIG. 21. In some embodiments, the LNP comprising a compound described herein increases transfection efficiency of a cell by at least 0.1 fold, 0.2 fold, 0.3 fold, 0.4 fold, 0.5 fold, 1.0 fold, 2.0 fold, 5.0 fold, 10.0 fold, or more compared to a transfection efficiency of a cell by a comparable LNP without the compound. In some embodiments, an LNP comprising a compound described herein can transfect a cell without decreasing the viability of the transfected cell. For example, FIG. 2, FIG. 4, FIG. 6, FIG. 11, FIG. 22, FIG. 24, and FIG. 26 illustrate comparable cell viability of the LNP comprising a compound described herein as compared to cell viability of a cell transfected by a comparable LNP without the compound.

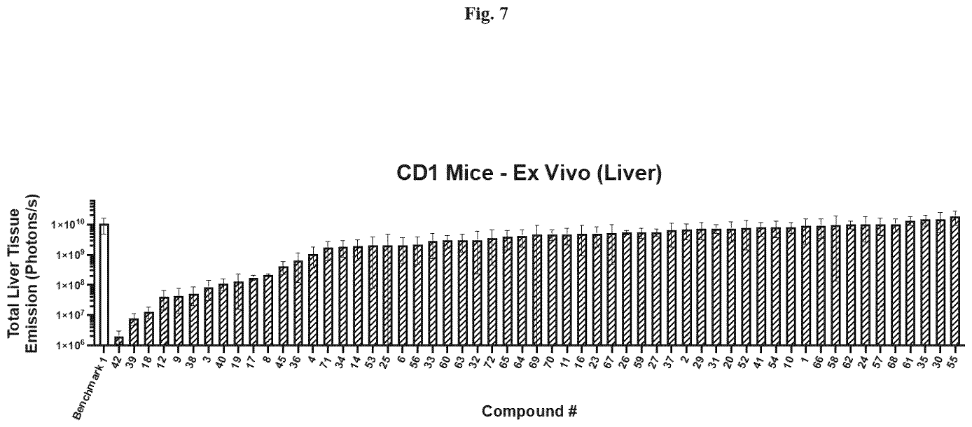

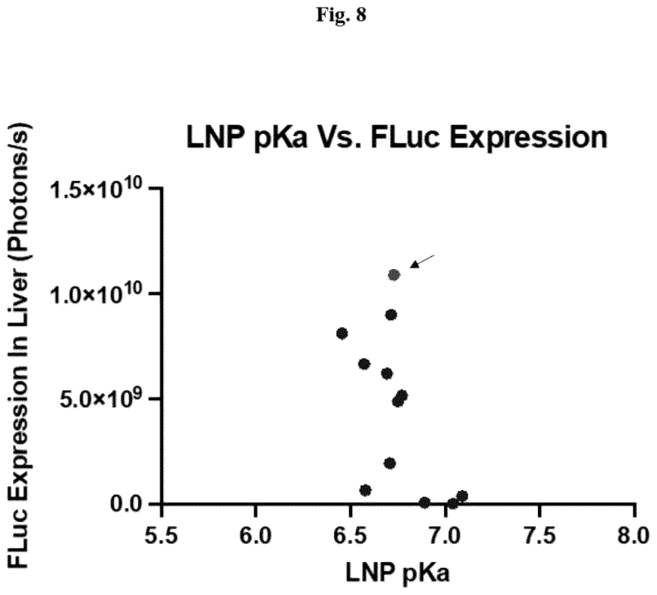

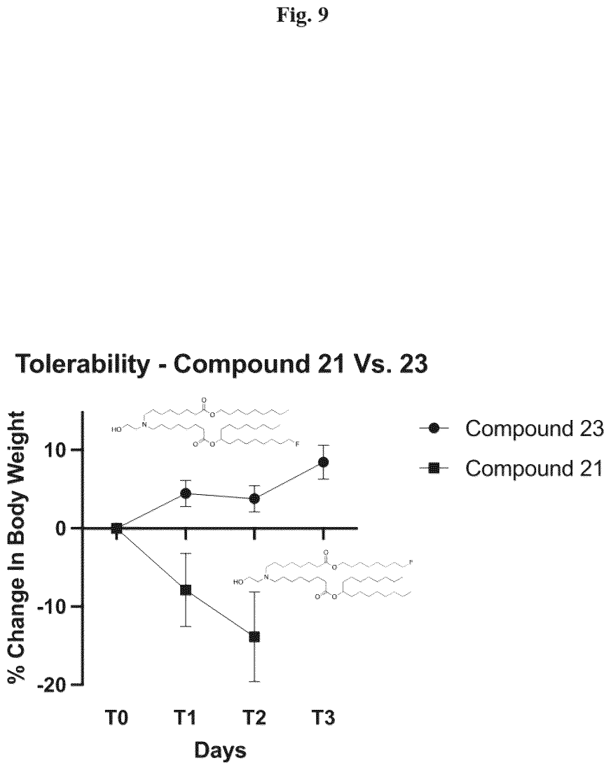

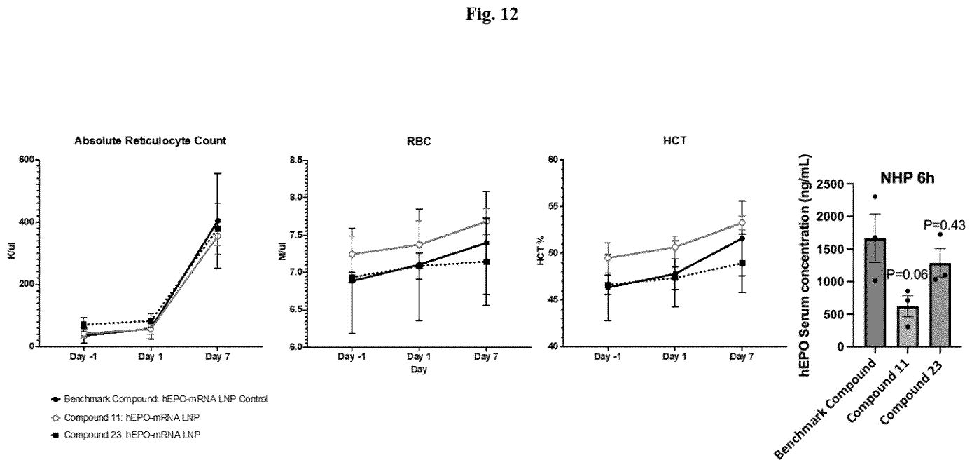

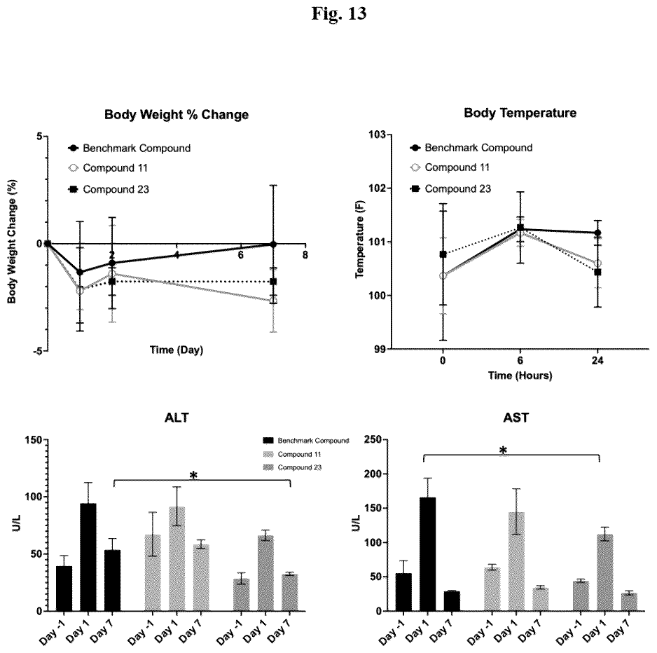

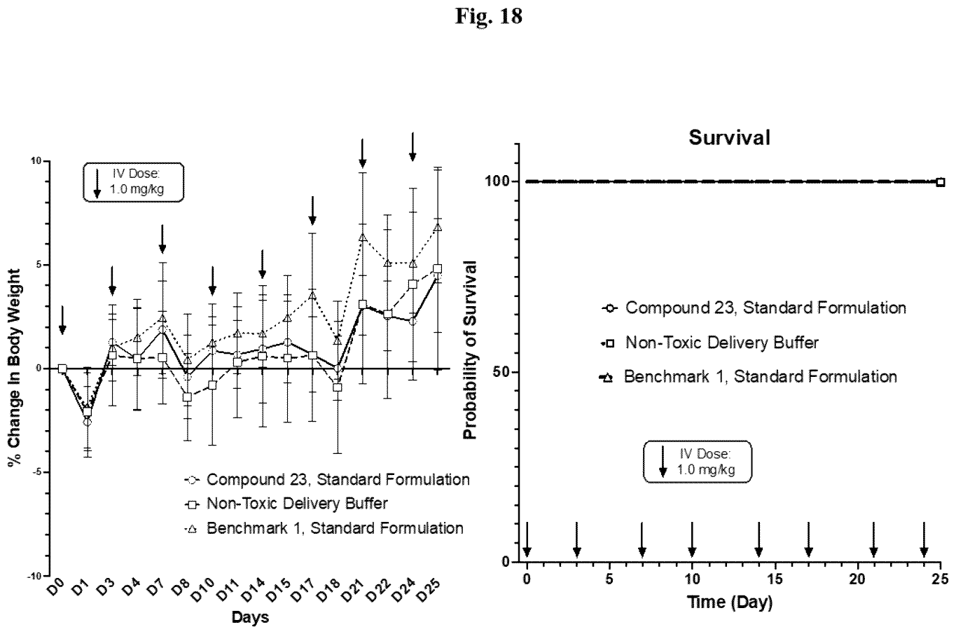

In some embodiments, an LNP comprising a compound described herein can transfect a cell ex vivo at a comparable level as a comparable LNP without the compound. For example, FIG. 7 and FIG. 8 illustrate comparable transfection efficiency of ex vivo cells by an LNP comprising a compound described herein as compared to a benchmark lipid (e.g., a lipid without the compound described herein, Benchmark 1). In some embodiments, the LNP comprising the compound does not lead to in vivo toxicity (e.g., as shown in FIG. 9, FIG. 13, and FIG. 18). In some embodiments, an LNP comprising a compound described herein can transfect a cell in vivo at a comparable level as a comparable LNP without the compound. For example, FIG. 12 illustrates comparable transfection efficiency of in vivo cells by an LNP comprising a compound described herein as compared to a benchmark lipid (e.g., a lipid without the compound described herein, Benchmark 1).

In one aspect, disclosed herein are methods for treating a disease or condition in a subject. In one embodiment, the disease or condition is treatable by administering the payload. In some embodiments, the disease or condition is cancer or associated with cancer. A payload included in an LNP composition may also be capable of altering the rate of transcription of a given species, thereby affecting gene expression.

In some embodiments, the nanoparticle compositions (e.g., nanoparticle composition comprising a compound described herein), LNP compositions (e.g., LNP comprising a compound described herein), or pharmaceutical compositions disclosed herein is administered at least once during a period of time (e.g., every 2 days, twice a week, once a week, every week, three times per month, two times per month, one time per month, every 2 months, every 3 months, every 4 months, every 5 months, every 6 months, every 7 months, every 8 months, every 9 months, every 10 months, every 11 months, once a year). In some embodiments, the composition is administered two or more times (e.g., 2, 3, 4, 5, 6, 7, 8, 9, 10, 11, 12, 13, 14, 15, 16, 17, 18, 19, 20, 30, 40, 50, 60, 70, 80, 90, 100 times) during a period of time. In some embodiments, the method comprises administering the compounds, nanoparticle compositions (e.g., nanoparticle composition comprising a compound described herein), LNP compositions (e.g., LNP comprising a compound described herein), or pharmaceutical compositions disclosed herein in a therapeutically-effective amount by various forms and routes including, for example, intratumoral, oral, or topical administration. In some embodiments, a composition may be administered by intratumoral, parenteral, intravenous, subcutaneous, intramuscular, intradermal, intraperitoneal, intracerebral, subarachnoid, intraocular, intrasternal, ophthalmic, endothelial, local, intranasal, intrapulmonary, rectal, intraarterial, intrathecal, inhalation, intralesional, intradermal, epidural, intracapsular, subcapsular, intracardiac, transtracheal, subcuticular, subarachnoid, or intraspinal administration, e.g., injection or infusion. In some embodiments, a composition may be administered by absorption through epithelial or mucocutaneous linings (e.g., oral mucosa, rectal and intestinal mucosa administration). In some embodiments, the compounds, nanoparticle compositions (e.g., nanoparticle composition comprising a compound described herein), LNP compositions (e.g., LNP comprising a compound described herein), or pharmaceutical compositions disclosed herein is delivered via multiple administration routes.

Actual dosage levels of an agent of the disclosure may be varied so as to obtain an amount of the agent to achieve the desired therapeutic response for a particular subject, composition, and mode of administration, without being toxic to the subject (e.g., the subject for immunization or the subject for treatment). The selected dosage level may depend upon a variety of pharmacokinetic factors including the activity of the particular compositions of the present invention employed, the route of administration, the time of administration, the rate of excretion, the duration of the treatment, other drugs, compounds and/or materials used in combination with the particular compositions employed, the age, sex, weight, condition, general health and prior medical history of the patient being treated, and like factors well known in the medical arts.

Dosage regimens may be adjusted to provide the optimum desired response (e.g., a therapeutic and/or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects (e.g., the subjects for immunization or the subjects for treatment); each unit contains a predetermined quantity of active agent calculated to produce the desired therapeutic effect in association with the required pharmaceutical carrier. The specification for the dosage unit forms of the disclosure may be determined by and directly dependent on (a) the unique characteristics of the active agent and the particular therapeutic effect to be achieved, and (b) the limitations inherent in the art of compounding such an active agent for the treatment of sensitivity in individuals. A dose may be determined by reference to a plasma concentration or a local concentration of the circular polyribonucleotide or antibody or antigen-binding fragment thereof. A dose may be determined by reference to a plasma concentration or a local concentration of the linear polyribonucleotide or antibody or antigen-binding fragment thereof.

The compounds, nanoparticle compositions (e.g., nanoparticle composition comprising a compound described herein), LNP compositions (e.g., LNP comprising a compound described herein), or pharmaceutical compositions disclosed herein may be in a unit dosage form suitable for a single administration of a precise dosage. In unit dosage form, the formulation may be divided into unit doses containing appropriate quantities of the compositions. In unit dosage form, the formulation may be divided into unit doses containing appropriate quantities of one or more linear polyribonucleotides, antibodies or the antigen-binding fragments thereof, and/or therapeutic agents. The unit dosage may be in the form of a package containing discrete quantities of the formulation. Non-limiting examples are packaged injectables, vials, and ampoules. An aqueous suspension composition disclosed herein may be packaged in a single-dose non-reclosable container. Multiple-dose reclosable containers may be used, for example, in combination with or without a preservative. A formulation for injection disclosed herein may be present in a unit dosage form, for example, in ampoules, or in multi dose containers with a preservative.

In some embodiments, described herein is a method of delivering a therapeutic agent to a subject in need thereof, the method comprising administering to the subject a nanoparticle composition described herein or a pharmaceutical composition described herein, thereby the therapeutic agent is delivered to the subject. In some embodiments, the therapeutic agent is a payload described herein. In some embodiments, the therapeutic agent comprises a nucleic acid described herein (e.g., an mRNA). In some embodiments, the therapeutic agent encodes a polypeptide of interest (e.g., a cytokine). In some embodiments, the therapeutic agent can be expressed by a cell contacted with the nanoparticle composition described herein or a pharmaceutical composition described herein. In some embodiments, the method comprises selectively delivering a therapeutic agent to a mammalian organ. In some embodiments, the method comprises selectively expressing a therapeutic agent in a cell. For example, the therapeutic agent comprises an oncoselective motif or an oncoselective element for selectively expressing therapeutic agent in a cell associated with a disease or condition (e.g., a cancer cell). In some embodiments, the method treats a disease or condition in a subject. In some embodiments, the disease or disorder is cancer.

The terminology used herein is for the purpose of describing particular cases only and is not intended to be limiting. In this application, the use of the singular includes the plural unless specifically stated otherwise. As used herein, the singular forms “a”, “an” and “the” are intended to include the plural forms as well, unless the context clearly indicates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments.” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

The term “derivative” as used herein indicates a chemical or biological substance that is related structurally to a second substance and derivable from the second substance through a modification of the second substance. In particular, if a first compound is a derivative of a second compound and the second compound is associated with a chemical and/or biological activity, the first compound differs from the second compound for at least one structural feature, while retaining (at least to a certain extent) the chemical and/or biological activity of the second compound and at least one structural feature (e.g. a sequence, a fragment, a functional group and others) associated thereto. A skilled person will be able to identify, on a case by case basis and upon reading of the present disclosure, structural features of the second compound that have to be maintained in the first compound to retain the second compound chemical and/or biological activity as well as assays that can be used to prove retention of the chemical and/or biological activity. Exemplary “derivatives” can include a prodrug, a metabolite, an enantiomer, a diastereomer, esters (e.g. acyloxyalkyl esters, alkoxycarbonyloxyalkyl esters, alkyl esters, aryl esters, phosphate esters, sulfonate esters, sulfate esters and disulfide containing esters), ethers, amides, carbonates, thiocarbonates, N-acyl derivatives, N-acyloxyalkyl derivatives, quaternary derivatives of tertiary amines, N-Mannich bases, Schiff bases, amino acid conjugates, phosphate esters, metal salts, sulfonate esters, and the like. In some cases, a derivative may include trivial substitutions (i.e. additional alkyl/akylene groups) to a parent compound that retains the chemical and/or biological activity of the parent compound.

The term “pharmaceutically acceptable” refers to approved or approvable by a regulatory agency of the Federal or a state government or listed in the U.S. Pharmacopeia or other generally recognized pharmacopeia for use in animals, including humans.

A “pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent.

A “pharmaceutically acceptable salt” may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethyl sulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)n-COOH where n is 0-4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts include those listed by Remington's Pharmaceutical Sciences, 17th ed., Mack Publishing Company, Easton, PA, p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

As used herein, the term “therapeutically effective amount” means an amount of an agent to be delivered (e.g., nucleic acid, drug, payload, composition, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The term “subject” refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline.

The terms “treat,” “treated,” “treating,” “treatment,” and the like are meant to refer to reducing or ameliorating a disorder and/or symptoms associated therewith (e.g., a neoplasia or tumor). “Treating” may refer to administration of the LNP composition to a subject after the onset, or suspected onset, of a disease or condition. “Treating” includes the concepts of “alleviating”, which refers to lessening the frequency of occurrence or recurrence, or the severity, of any symptoms or other ill effects related to a disease or condition and/or the side effects associated with the disease or condition. The term “treating” also encompasses the concept of “managing” which refers to reducing the severity of a particular disease or disorder in a patient or delaying its recurrence, e.g., lengthening the period of remission in a patient who had suffered from the disease. It is appreciated that, although not precluded, treating a disorder or condition does not require that the disorder, condition, or symptoms associated therewith be completely eliminated.

The term “substituted”, unless otherwise indicated, refers to the replacement of one or more hydrogen radicals in a given structure with the radical of a specified substituent including, but not limited to: halo, alkyl, alkenyl, alkynyl, aryl, heterocyclyl, thiol, alkylthio, oxo, thioxy, arylthio, alkylthioalkyl, arylthioalkyl, alkylsulfonyl, alkylsulfonylalkyl, arylsulfonylalkyl, alkoxy, aryloxy, aralkoxy, aminocarbonyl, alkylaminocarbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, hydroxy, alkoxyalkyl, carboxyalkyl, alkoxycarbonylalkyl, aminocarbonylalkyl, acyl, aralkoxycarbonyl, carboxylic acid, sulfonic acid, sulfonyl, phosphonic acid, aryl, heteroaryl, heterocyclic, and an aliphatic group. It is understood that the substituent may be further substituted. Exemplary substituents include amino, alkylamino, and the like.

As used herein, the term “substituent” means positional variables on the atoms of a core molecule that are substituted at a designated atom position, replacing one or more hydrogens on the designated atom, provided that the designated atom's normal valency is not exceeded, and that the substitution results in a stable compound. Combinations of substituents and/or variables are permissible only if such combinations result in stable compounds. A person of ordinary skill in the art should note that any carbon as well as heteroatom with valences that appear to be unsatisfied as described or shown herein is assumed to have a sufficient number of hydrogen atom(s) to satisfy the valences described or shown. In certain instances one or more substituents having a double bond (e.g., “oxo” or “=0”) as the point of attachment may be described, shown or listed herein within a substituent group, wherein the structure may only show a single bond as the point of attachment to the core structure of Formula (I), Formula (Ia), Formula (II), Formula (IIa), Formula (III), or Formula (IIIa). A person of ordinary skill in the art would understand that, while only a single bond is shown, a double bond is intended for those substituents.

The term “alkyl” refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl, likewise, for example, an alkyl comprising up to 6 carbon atoms is a C₁-C₆ alkyl. Alkyls (and other moieties defined herein) comprising other numbers of carbon atoms are represented similarly. Alkyl groups include, but are not limited to, C₁-C₁₄ alkyl, C₁-C₁₃ alkyl, C₁-C₁₂ alkyl, C₁-C₁₁ alkyl, C₁-C₁₀ alkyl, C₁-C₉ alkyl, C₁-C₈alkyl, C₁-C₇ alkyl, C₁-C₆ alkyl, C₁-C₅alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, n-propyl, 1-methylethyl (i-propyl), n-butyl, i-butyl, s-butyl, n-pentyl, 1,1-dimethylethyl (1-butyl), 3-methylhexyl, 2-methylhexyl, 1-ethyl-propyl, and the like. In some embodiments, the alkyl is methyl or ethyl. In some embodiments, the alkyl is —CH(CH₃)₂ or —C(CH₃)₃. Unless stated otherwise specifically in the specification, an alkyl group may be optionally substituted as described below. “Alkylene” or “alkylene chain” refers to a straight or branched divalent hydrocarbon chain linking the rest of the molecule to a radical group. In some embodiments, the alkylene is —CH₂—, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—.

The term “heteroalkyl” refers to an alkyl group in which one or more skeletal atoms of the alkyl are selected from an atom other than carbon, e.g., oxygen, nitrogen (e.g. —NH—, —N(alkyl)-, sulfur, or combinations thereof. A heteroalkyl is attached to the rest of the molecule at a carbon atom of the heteroalkyl. In one aspect, a heteroalkyl is a C₁-C₆ heteroalkyl. Heteroalkyl may include nitriles, amides, esters, ethers, amines, thioethers, thioesters, carbamates, carbonates, polyethers, polyamines, and the like.

The term “halo” or, alternatively, “halogen” or “halide” means fluoro, chloro, bromo or iodo. In some embodiments, halo is fluoro, chloro, or bromo. When used as a prefix, halo does not denote any number of instances. For example, haloalkyl includes methyl derivatives —CH₂F, —CHF₂, and —CF₃.

The term “haloalkyl” refers to an alkyl group wherein at least one, and possibly more, hydrogen atoms have been replaced with a halogen. For example, haloalkyl includes methyl derivatives —CH₂F, —CHF₂, and —CF₃. Haloalkyl is non-limiting in terms of number of halogens and carbons. Generally, haloalkyl refers to C₁-C₁₂ haloalkyl.

The term “aryl” refers to a radical derived from a hydrocarbon ring system comprising at least one aromatic ring. In some embodiments, an aryl comprises hydrogens and 6 to 30 carbon atoms. The aryl radical can be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl. Aryl radicals include, but are not limited to, aryl radicals derived from the hydrocarbon ring systems of anthrylene, naphthylene, phenanthrylene, anthracene, azulene, benzene, chrysene, fluoranthene, fluorene, indane, indene, naphthalene, phenalene, phenanthrene, pleiadene, pyrene, and triphenylene. In some embodiments, the aryl is phenyl. Unless stated otherwise specifically in the specification, an aryl can be optionally substituted, for example, with halogen, amino, alkylamino, aminoalkyl, nitrile, nitro, hydroxyl, alkyl, alkenyl, alkynyl, haloalkyl, heteroalkyl, alkoxy, aryl, cycloalkyl, heterocycloalkyl, heteroaryl, —S(O)₂NH—C₁-C₆alkyl, and the like. In some embodiments, an aryl is optionally substituted with halogen, methyl, ethyl, —CN, —CF₃, —OH, —OMe, —NH₂, —NO₂, —S(O)₂NH₂, —S(O)₂NHCH₃, —S(O)₂NHCH₂CH₃, —S(O)₂NHCH(CH₃)₂, —S(O)₂N(CH₃)₂, or —S(O)₂NHC(CH₃)₃. In some embodiments, an aryl is optionally substituted with halogen, methyl, ethyl, —CN, —CF₃, —OH, or —OMe. In some embodiments, the aryl is optionally substituted with halogen. In some embodiments, the aryl is substituted with alkyl, alkenyl, alkynyl, haloalkyl, or heteroalkyl, wherein each alkyl, alkenyl, alkynyl, haloalkyl, heteroalkyl is independently unsubstituted, or substituted with halogen, methyl, ethyl, —CN, —CF₃, —OH, —OMe, —NH₂, or —NO₂.

The term “alkenyl” refers to a type of alkyl group in which at least one carbon-carbon double bond is present. In one embodiment, an alkenyl group has the formula —C(R^{a})═CR^{a}₂, wherein R^{a} refers to the remaining portions of the alkenyl group, which may be the same or different. In some embodiments, R^{a} is H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)═CH₂, —CH═CHCH₃, —C(CH₃)—CHCH₃, and —CH₂CH═CH₂. “Alkenylene” or “alkenylene chain” refers to a alkylene group in which at least one carbon-carbon double bond is present. In some embodiments, the alkenylene is —CH═CH—, —CH₂CH₂CH═CH—, or —CH═CHCH₂CH₂—. In some embodiments, the alkenylene is —CH═CH—. In some embodiments, the alkenylene is —CH₂CH₂CH═CH—. In some embodiments, the alkenylene is —CH═CHCH₂CH₂—.

The term “alkynyl” refers to a type of alkyl group in which at least one carbon-carbon triple bond is present. In one embodiment, an alkynyl group has the formula —C≡CR^{a}, wherein R^{a} refers to the remaining portions of the alkynyl group. In some embodiments, R^{a} is H or an alkyl. In some embodiments, an alkynyl is selected from ethynyl (i.e., acetylenyl), propynyl (i.e., propargyl), butynyl, pentynyl, and the like. Non-limiting examples of an alkynyl group include —C≡CH, —C≡CCH₃, and —CH₂C≡CH. “Alkynylene” or “alkynylene chain” refers to a alkylene group in which at least one carbon-carbon triple bond is present. In some embodiments, the alkynylene is —C≡C—, —CH₂CH₂C≡C—, or —C≡CCH₂CH₂—. In some embodiments, the alkynylene is —C≡C—. In some embodiments, the alkynylene is —CH₂CH₂C≡C—. In some embodiments, the alkynylene is —C≡CCH₂CH₂—.

The term “alkenylalkylene” refers to a straight or branched hydrocarbon chain, containing at least one carbon-carbon double bond. In certain embodiments, alkenyl comprises two to fifteen (C₂-C₁₅ alkenylalkylene) carbon atoms, or two to twelve carbon atoms (C₂-C₁₂ alkenylalkylene), or three to twelve carbon atoms (C₃-C₁₂ alkenylalkylene), or three to ten carbon atoms (C₃-C₁₀ alkenylalkylene), and the like. The alkenylalkylene may be attached to the rest of the molecule by two single bonds, non-limiting examples of alkenylalkylene are: —CH₂CH═CH—, —CH₂CH—CHCH₂—, —CH₂ (CH₂)₅CH═CH—, —CH₂(CH₂)₄CH═CH(CH₂)₂CH₂—, —CH₂(CH₂)₁₁CH═CH—, and the like. In some embodiments, the alkenylalkylene is a hydrocarbon chain, containing one to five carbon-carbon double bonds. In some embodiments, the alkenylalkylene is a hydrocarbon chain, containing one carbon-carbon double bond. In some embodiments, the alkenylalkylene is a hydrocarbon chain, containing two carbon-carbon double bonds. In some embodiments, the alkenylalkylene is a hydrocarbon chain, containing three carbon-carbon double bonds. In some embodiments, the alkenylalkylene is a hydrocarbon chain, containing four carbon-carbon double bonds. In some embodiments, the alkenylalkylene is a hydrocarbon chain, containing five carbon-carbon double bonds. In certain embodiments, an alkenyl group is optionally substituted by one or more of the following substituents: halogen, deuterium, —CN, linear or branched C₁₋₁₀ alkyl, linear or branched C₁₋₁₀ heteroalkyl, C₁₋₂ haloalkyl, —C(═O)R¹⁰, —C(═O)N(R¹⁰)₂, —OR¹⁰, —N(R¹⁰)₂, substituted or unsubstituted C₃₋₆ cycloalkyl, and substituted or unsubstituted 3- to 10-membered heterocycloalkyl; wherein each substituted C₃₋₆ cycloalkyl and substituted 3- to 10-membered heterocycloalkyl is substituted with 1-5 R¹¹; wherein R¹⁰ is independently hydrogen, C₁₋₁₀ alkyl, C₁₋₁₀ heteroalkyl, C₁₋₁₀ haloalkyl, C₃₋₁₀ cycloalkyl, C₃₋₁₀ heterocycloalkyl, C₂₋₆ alkenyl, or C₂₋₆ alkynyl; and each R¹¹ is independently hydrogen, deuterium, halogen, —CN, C₁₋₁₀ alkyl, C₁₋₁₀ heteroalkyl, C₃₋₁₀ cycloalkyl, C₃₋₁₀ heterocycloalkyl, C₂₋₆ alkenyl, C₂₋₆ alkynyl, —C(O)OH, —C(O)—O—C₁₋₆ alkyl, —C(O)—C₁₋₆ alkyl, —C(O)NH₂, —C(O)NH(C₁₋₆ alkyl), —C(O)N(C₁₋₆ alkyl)₂, —NH₂, —NH(C₁₋₆ alkyl), —N(C₁₋₆ alkyl)₂, —OH, —O—C₁₋₆ alkyl, —SH, —S(C₁₋₆ alkyl), —S(O)(C₁₋₆ alkyl), —S(O)₂(C₁₋₆ alkyl), or —S(O)₂NH(C₁₋₆ alkyl)

The term “cycloalkyl” refers to a monocyclic or polycyclic non-aromatic radical, wherein each of the atoms forming the ring (i.e. skeletal atoms) is a carbon atom. In some embodiments, cycloalkyls are saturated or partially unsaturated. In some embodiments, cycloalkyls are spirocyclic or bridged compounds. In some embodiments, cycloalkyls are fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cycloalkyl radicals include, for example, cyclopropyl, cyclobutyl, cyclopentyl, cyclohexyl, cycloheptyl, and cyclooctyl. In some embodiments, the monocyclic cycloalkyl is cyclopropyl, cyclobutyl, cyclopentyl or cyclohexyl. In some embodiments, the monocyclic cycloalkyl is cyclopentenyl or cyclohexenyl. In some embodiments, the monocyclic cycloalkyl is cyclopentenyl. Polycyclic radicals include, for example, adamantyl, 1,2-dihydronaphthalenyl, 1,4-dihydronaphthalenyl, tetrainyl, decalinyl, 3,4-dihydronaphthalenyl-1(2H)-one, spiro[2.2]pentyl, norbornyl and bicycle[1.1.1]pentyl. Unless otherwise stated specifically in the specification, a cycloalkyl group may be optionally substituted. Depending on the structure, a cycloalkyl group can be monovalent or divalent (i.e., a cycloalkylene group).

The term “heterocycle” or “heterocyclic” refers to heteroaromatic rings (also known as heteroaryls) and heterocycloalkyl rings (also known as heteroalicyclic groups) that includes at least one heteroatom selected from nitrogen, oxygen and sulfur, wherein each heterocyclic group has from 3 to 12 atoms in its ring system, and with the proviso that any ring does not contain two adjacent O or S atoms. A “heterocyclyl” is a univalent group formed by removing a hydrogen atom from any ring atoms of a heterocyclic compound. In some embodiments, heterocycles are monocyclic, bicyclic, polycyclic, spirocyclic or bridged compounds. Non-aromatic heterocyclic groups (also known as heterocycloalkyls) include rings having 3 to 12 atoms in its ring system and aromatic heterocyclic groups include rings having 5 to 12 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3 h-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The term “heterocycloalkyl” refers to a cycloalkyl group that includes at least one ring heteroatom selected from nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quaternized. The heterocycloalkyl radical is partially or fully saturated. Examples of heterocycloalkyl radicals include, but are not limited to, dioxolanyl, thienyl[1,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. In some embodiments, heterocycloalkyls have from 2 to 10 carbons in the ring. In some embodiments, heterocycloalkyls have from 2 to 10 carbons in the ring and 1 or 2 N atoms. In some embodiments, heterocycloalkyls have from 2 to 10 carbons in the ring and 3 or 4 N atoms. In some embodiments, heterocycloalkyls have from 2 to 12 carbons, 0-2 N atoms, 0-2 O atoms, 0-2 P atoms, and 0-1 S atoms in the ring. In some embodiments, heterocycloalkyls have from 2 to 12 carbons, 1-3 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group may be optionally substituted. As used herein, the term “heterocycloalkylene” can refer to a divalent heterocycloalkyl group.

The term “heteroaryl” refers to an aryl group that includes one or more ring heteroatoms selected from nitrogen, oxygen and sulfur. The heteroaryl is monocyclic or bicyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-6 N atoms in the ring. In some embodiments, a heteroaryl contains 1-4 N atoms in the ring. In some embodiments, a heteroaryl contains 4-6 N atoms in the ring. In some embodiments, a heteroaryl contains 0-4 N atoms, 0-1 O atoms, 0-1 P atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉ heteroaryl. In some embodiments, monocyclic heteroaryl is a C₁-C₅ heteroaryl. In some embodiments, monocyclic heteroaryl is a 5-membered or 6-membered heteroaryl. In some embodiments, a bicyclic heteroaryl is a C₆-C₉ heteroaryl. In some embodiments, a heteroaryl group is partially reduced to form a heterocycloalkyl group defined herein. In some embodiments, a heteroaryl group is fully reduced to form a heterocycloalkyl group defined herein.

As used herein, the “N/P ratio” is the molar ratio of ionizable (e.g., in the physiological pH range) nitrogen atoms in a lipid (or lipids) to phosphate groups in a nucleic acid molecular entity (or nucleic acid molecular entities), e.g., in a nanoparticle composition comprising a lipid component and an RNA. Ionizable nitrogen atoms can include, for example, nitrogen atoms that can be protonated at about pH 1, about pH 2, about pH 3, about pH 4, about pH5, about pH 6, about pH 7, about pH 7.5, or about pH 8 or higher. The physiological pH range can include, for example, the pH range of different cellular compartments (such as organs, tissues, and cells) and bodily fluids (such as blood, CSF, gastric juice, milk, bile, saliva, tears, and urine). In certain specific embodiments, the physiological pH range refers to the pH range in a mammal, for example, from about 7.35 to about 7.45. In some embodiments, ionizable nitrogen atoms refer to those nitrogen atoms that are ionizable within a pH range between 5 and 14.

For the payload that does not contain a phosphate group, the N/P ratio can refer to a molar ratio of ionizable nitrogen atoms in a lipid to the total negative charge in the payload. For example, the N/P ratio of an LNP composition can refer to a molar ratio of the total ionizable nitrogen atoms in the LNP composition to the total negative charge in the payload that is present in the composition.

As used herein, amino lipids can contain at least one primary, secondary or tertiary amine moiety that is protonatable (or ionizable) between pH range 4 and 14. In some embodiments, and the amine moiety/moieties function as the hydrophilic headgroup of the amino lipids described in Tables 1-3. When most of the amine moiety(ies) of an amino lipid (or amino lipids) in a nucleic acid-lipid nanoparticle formulation is protonated at physiological pH, then the nanoparticles can be termed as cationic lipid nanoparticle (cLNP). When most of the amine moiety(ies) of an amino lipid (or amino lipids) in a nucleic acid-lipid nanoparticle formulation is not protonated at physiological pH but can be protonated at acidic pH, endosomal pH for example, can be termed as ionizable lipid nanoparticle (iLNP). The amino lipids that constitute cLNPs can be generally called cationic amino lipids (cLipids). The amino lipids that constitute iLNPs can be called ionizable amino lipids (iLipids). The amino lipids described in Tables 1-3 can be an iLipid or a cLipid at physiological pH.

As used herein, a “lipid nanoparticle (LNP) composition” or a “nanoparticle composition” is a composition comprising one or more described lipids. LNP compositions are typically sized on the order of micrometers or smaller and may include a lipid bilayer.

Nanoparticle compositions encompass lipid nanoparticles (LNPs), liposomes (e.g., lipid vesicles), and lipoplexes. For example, a nanoparticle composition may be a liposome having a lipid bilayer with a diameter of 500 nm or less. The LNPs described herein can have a mean diameter of from about 1 nm to about 2500 nm, from about 10 nm to about 1500 nm, from about 20 nm to about 1000 nm, from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, or from about 70 nm to about 80 nm. The LNPs described herein can have a mean diameter of about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, or greater. The LNPs described herein can be substantially non-toxic.

As used herein, a “PEG lipid” or “PEG-lipid” refers to a lipid comprising a polyethylene glycol component.

As used herein, a “phospholipid” can refer to a lipid that includes a phosphate moiety and one or more carbon chains, such as unsaturated fatty acid chains. A phospholipid may include one or more multiple (e.g., double or triple) bonds. In some embodiments, a phospholipid may facilitate fusion to a membrane. For example, a cationic phospholipid may interact with one or more negatively charged phospholipids of a membrane (e.g., a cellular or intracellular membrane). Fusion of a phospholipid to a membrane may allow one or more elements of an LNP to pass through the membrane, i.e., delivery of the one or more elements to a cell.

The term “therapeutic agent” can refer to any agent that, when administered to a subject, has a therapeutic, diagnostic, and/or prophylactic effect and/or elicits a desired biological and/or pharmacological effect. Therapeutic agents can also be referred to as “actives” or “active agents.” Such agents include, but are not limited to, cytotoxins, radioactive ions, chemotherapeutic agents, small molecule drugs, proteins, and nucleic acids.

As used herein, the term “nucleic acid” is used interchangeably with “nucleic acid molecular entity.” The term “nucleic acid” as used herein refers to a polymer containing at least two nucleotides (i.e., deoxyribonucleotides or ribonucleotides) in either single- or double-stranded form and includes DNA and RNA. “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs and/or modified residues include, without limitation, phosphorothioates, phosphoramidates, methyl phosphonates, chiral-methyl phosphonates, 2′-O-methyl ribonucleotides, and peptide-nucleic acids (PNAs).

The term “nucleic acid” includes any oligonucleotide or polynucleotide, with fragments containing up to 60 nucleotides generally termed oligonucleotides, and longer fragments termed polynucleotides. A deoxyribooligonucleotide consists of a 5-carbon sugar called deoxyribose joined covalently to phosphate at the 5′ and 3′ carbons of this sugar to form an alternating, unbranched polymer. DNA may be in the form of, e.g., antisense molecules, plasmid DNA, pre-condensed DNA, a PCR product, vectors, expression cassettes, chimeric sequences, chromosomal DNA, or derivatives and combinations of these groups. A ribooligonucleotide consists of a similar repeating structure where the 5-carbon sugar is ribose. Accordingly, the terms “polynucleotide” and “oligonucleotide” can refer to a polymer or oligomer of nucleotide or nucleoside monomers consisting of naturally-occurring bases, sugars and intersugar (backbone) linkages. The terms “polynucleotide” and “oligonucleotide” can also include polymers or oligomers comprising non-naturally occurring monomers, or portions thereof, which function similarly. Such modified or substituted oligonucleotides are often preferred over native forms because of properties such as, for example, enhanced cellular uptake, reduced immunogenicity, and increased stability in the presence of nucleases.

Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)).

The present disclosure encompasses isolated or substantially purified nucleic acid molecules and compositions containing those molecules. As used herein, an “isolated” or “purified” DNA molecule or RNA molecule is a DNA molecule or RNA molecule that exists apart from its native environment. An isolated DNA molecule or RNA molecule may exist in a purified form or may exist in a non-native environment such as, for example, a transgenic host cell. For example, an “isolated” or “purified” nucleic acid molecule or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ends of the nucleic acid) in the genomic DNA of the organism from which the nucleic acid is derived. For example, in some embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived.

### Examples

### Example 1. Formulation Process

mRNA encapsulated lipid nanoparticles (mRNA LNPs) were formulated using microfluidic mixing with a PNI Ignite instrument. Briefly, lipids (ionizable: structural: helper: PEG) were dissolved in ethanol and mixed with and aqueous solution of mRNA. These mRNA-loaded dispersions were then neutralized via a buffer exchange with a pre-determined buffer overnight. Afterwards, formed LNPs were concentrated with Amicon filter centrifugation, filtered, concentration of mRNA determined through a ribogreen assay, and frozen at −80° C. until use.

### Example 2. TNS Assay

mRNA encapsulated lipid nanoparticles (mRNA LNPs) were formulated using microfluidic mixing with a PNI Ignite instrument. Briefly, lipids (ionizable: structural: helper: PEG) were dissolved in ethanol and mixed with and aqueous solution of mRNA. These mRNA-loaded dispersions were then neutralized via a buffer exchange with a pre-determined buffer overnight. Afterwards, formed LNPs were concentrated with Amicon filter centrifugation, filtered, concentration of mRNA determined through a ribogreen assay, and frozen at −80° C. until use.

### Example 3. In Vitro Experimental

Cell lines were grown to confluence in T75 flasks using complete media of DMEM-F12 for AML12 and DMEM for both B16F10 and MC38.K. Cells were plated in a white 96 flat-well plate (AML12=20,000 cells/well, B16F10 and MC38.K=5,000 cells/well) with their respected media. Lipid nanoparticles were first diluted to 50 μg/ml using Opti-MEM and pre-incubated with mouse serum for 30 minutes to give an LNP concentration of 8 μg/ml. Cell media was replaced with Opti-MEM and LNPs were added to cells (200 ng of mRNA-LNP), placed back in the incubator, and allowed to transfect for 24 hours. Afterwards, 100 μl of Promega's ONE-Glo™ EX Luciferase Assay System or Promega's CellTiter-Glo® Luminescent Cell Viability Assay was added and mixed in their respective wells, followed by recording luminescence signals on a Thermo Scientific Varioskan LUX multimode microplate reader. The results are shown in FIG. 1, FIG. 2, FIG. 3, FIG. 4, FIG. 5, FIG. 6, FIG. 10, FIG. 11, and FIG. 21. A significant difference in transfection as measured experimentally was observed for separate lipid formulations.

TABLE 4
Biological results of in vitro testing on
luciferin signal via photon measurement.

| | | | | Total Liver |
|---|---|---|---|---|
| | | | | Tissue |
| | | | | Luciferin |
| | Total Luciferin Signal (RLU) | | | Signal |
| Com- | In | In | In | (Photons/ |
| pound | Vitro - | Vitro - | Vitro - | s) |
| Number | AML12 | B16F10 | MC38.K | In Vitro - CD1 |
| Bench- | 1.58E+06 | 1.90E+06 | 2.48E+06 | 1.09E+10 |
| mark 1* | | | | |
| Com- | 1.96E+06 | 6.92E+06 | 7.83E+06 | 9.00E+09 |
| pound 1 | | | | |
| Com- | 4.76E+06 | 5.94E+06 | 4.24E+06 | 6.20E+09 |
| pound 2 | | | | |
| Com- | 3.82E+06 | 4.58E+06 | 2.99E+06 | 8.57E+07 |
| pound 3 | | | | |
| Com- | 1.72E+06 | 2.16E+06 | 3.54E+06 | 1.06E+09 |
| pound 4 | | | | |
| Com- | 2.04E+05 | 1.79E+06 | 4.09E+06 | — |
| pound 5 | | | | |
| Com- | 8.42E+05 | 5.77E+05 | 9.66E+05 | 2.10E+09 |
| pound 6 | | | | |
| Com- | 1.48E+06 | 1.11E+06 | 1.35E+06 | — |
| pound 7 | | | | |
| Com- | 1.43E+05 | 9.69E+05 | 3.40E+06 | 2.13E+08 |
| pound 8 | | | | |
| Com- | 6.57E+05 | 3.27E+06 | 1.23E+06 | 4.41E+07 |
| pound 9 | | | | |
| Com- | 7.51E+05 | 4.57E+05 | 6.38E+05 | 8.34E+09 |
| pound 10 | | | | |
| Com- | 4.29E+06 | 7.92E+06 | 7.64E+06 | 4.88E+09 |
| pound 11 | | | | |
| Com- | 7.43E+05 | 2.79E+06 | 4.07E+06 | 4.15E+07 |
| pound 12 | | | | |
| Com- | 1.44E+06 | 2.12E+06 | 7.83E+05 | — |
| pound 13 | | | | |
| Com- | 2.94E+06 | 5.12E+06 | 4.71E+06 | 1.93E+09 |
| pound 14 | | | | |
| Com- | 1.53E+06 | 1.97E+06 | 6.94E+05 | |
| pound 15 | | | | |
| Com- | 1.67E+06 | 7.67E+06 | 2.39E+06 | 5.11E+09 |
| pound 16 | | | | |
| Com- | 6.34E+05 | 2.64E+06 | 1.15E+06 | — |
| pound 17 | | | | |
| Com- | 2.99E+06 | 2.65E+06 | — | 4.74E+09 |
| pound 18 | | | | |
| Com- | 6.91E+05 | 1.27E+06 | 1.90E+06 | 1.28E+08 |
| pound 19 | | | | |
| Com- | 7.65E+05 | 4.65E+05 | 7.88E+05 | 7.61E+09 |
| pound 20 | | | | |
| Com- | 4.09E+05 | 1.03E+05 | 1.16E+06 | — |
| pound 21 | | | | |
| Com- | 1.24E+05 | 5.04E+05 | 2.65E+06 | — |
| pound 22 | | | | |
| Com- | 4.66E+05 | 9.60E+05 | 2.00E+06 | 5.15E+09 |
| pound 23 | | | | |
| Com- | 5.98E+05 | 3.37E+05 | 7.60E+05 | 1.04E+10 |
| pound 24 | | | | |
| Com- | 1.09E+05 | 4.43E+05 | 9.18E+05 | 2.05E+09 |
| pound 25 | | | | |
| Com- | 1.95E+05 | 1.73E+05 | 2.33E+05 | 5.54E+09 |
| pound 26 | | | | |
| Com- | 3.02E+05 | 6.19E+04 | 2.37E+05 | 5.60E+09 |
| pound 27 | | | | |
| Com- | 1.65E+05 | 2.34E+05 | 5.09E+05 | — |
| pound 28 | | | | |
| Com- | 8.52E+04 | 8.63E+05 | 2.97E+05 | 7.57E+09 |
| pound 29 | | | | |
| Com- | 8.06E+05 | 1.16E+05 | 4.65E+05 | 1.53E+10 |
| pound 30 | | | | |
| Com- | 1.09E+06 | 3.37E+06 | 4.78E+06 | 7.59E+09 |
| pound 31 | | | | |
| Com- | 1.53E+05 | 2.09E+05 | 4.43E+05 | 3.13E+09 |
| pound 32 | | | | |
| Com- | 1.38E+05 | 4.80E+05 | 1.07E+06 | 2.93E+09 |
| pound 33 | | | | |
| Com- | 1.56E+05 | 238754.8 | 4.19E+05 | 1.89E+09 |
| pound 34 | | | | |
| Com- | 1.57E+05 | 4.43E+05 | 4.52E+05 | 1.51E+10 |
| pound 35 | | | | |
| Com- | 2.18E+05 | 2.85E+05 | 2.32E+05 | 6.41E+08 |
| pound 36 | | | | |
| Com- | 7.63E+05 | 2.66E+06 | 1.00E+06 | 6.65E+09 |
| pound 37 | | | | |
| Com- | 1.22E+06 | 2.75E+06 | 2.06E+06 | 5.28E+07 |
| pound 38 | | | | |
| Com- | 6.92E+05 | 3.58E+05 | 8.82E+05 | 7.86E+06 |
| pound 39 | | | | |
| Com- | 4.13E+06 | 3.61E+06 | 8.09E+06 | 1.12E+08 |
| pound 40 | | | | |
| Com- | 3.63E+06 | 2.52E+06 | — | 8.12E+09 |
| pound 41 | | | | |
| Com- | 2.36E+04 | 1.91E+04 | 5.65E+04 | 2.04E+06 |
| pound 42 | | | | |
| Com- | Inherent Lipid Properties Result in No mRNA Encapsulation | | | |
| pound 43 | | | | |
| Com- | 8.87E+05 | 1.26E+06 | 3.17E+06 | — |
| pound 44 | | | | |
| Com- | 3.98E+05 | 1.98E+06 | 1.09E+06 | 4.18E+08 |
| pound 45 | | | | |
| Com- | 1.30E+06 | 1.48E+06 | 4.59E+06 | — |
| pound 46 | | | | |
| Com- | 3.52E+04 | 3.88E+04 | 5.18E+04 | — |
| pound 47 | | | | |
| Com- | 3.31E+06 | 4.45E+06 | 2.17E+06 | — |
| pound 48 | | | | |
| Com- | 1.75E+04 | 9.79E+03 | 2.99E+04 | — |
| pound 49 | | | | |
| Com- | 1.54E+04 | 1.30E+04 | 1.85E+04 | — |
| pound 50 | | | | |
| Com- | 1.01E+06 | 1.28E+06 | 1.70E+06 | — |
| pound 51 | | | | |
| Com- | 2.59E+05 | 2.53E+05 | 4.31E+05 | 7.87E+09 |
| pound 52 | | | | |
| Com- | 5.15E+06 | 2.06E+07 | 2.29E+07 | 2.03E+09 |
| pound 53 | | | | |
| Com- | 1.05E+06 | 7.25E+05 | 5.12E+06 | 8.29E+09 |
| pound 54 | | | | |
| Com- | 4.19E+05 | 1.10E+06 | 1.80E+06 | 1.93E+10 |
| pound 55 | | | | |
| Com- | 6.63E+06 | 1.54E+07 | 2.52E+07 | 1.77E+09 |
| pound 56 | | | | |
| Com- | 2.63E+06 | 1.27E+06 | 1.42E+07 | 1.05E+10 |
| pound 57 | | | | |
| Com- | 2.18E+06 | 4.03E+05 | 4.80E+06 | 9.65E+09 |
| pound 58 | | | | |
| Com- | 8.24E+05 | 6.17E+05 | 8.40E+06 | 5.55E+09 |
| pound 59 | | | | |
| Com- | 8.00E+05 | 3.17E+05 | 5.21E+06 | 3.00E+09 |
| pound 60 | | | | |
| Com- | 8.13E+05 | 9.46E+05 | 1.11E+07 | 1.38E+10 |
| pound 61 | | | | |
| Com- | 2.33E+06 | 2.73E+06 | 3.47E+07 | 1.03E+10 |
| pound 62 | | | | |
| Com- | 5.69E+05 | 6.91E+05 | 1.35E+07 | 3.02E+09 |
| pound 63 | | | | |
| Com- | 1.02E+06 | 1.10E+06 | 2.41E+07 | 4.39E+09 |
| pound 64 | | | | |
| Com- | 3.24E+05 | 3.78E+05 | 3.81E+06 | 4.01E+09 |
| pound 65 | | | | |
| Com- | 1.16E+06 | 1.12E+06 | 5.75E+06 | 9.44E+09 |
| pound 66 | | | | |
| Com- | 4.35E+06 | 2.75E+06 | 3.24E+07 | 5.31E+09 |
| pound 67 | | | | |
| Com- | 2.89E+06 | 2.83E+06 | 1.60E+07 | 1.06E+10 |
| pound 68 | | | | |
| Com- | 8.05E+06 | 3.62E+06 | 4.67E+07 | 4.64E+09 |
| pound 69 | | | | |
| Com- | 7.41E+05 | 7.68E+05 | 5.15E+06 | 4.87E+09 |
| pound 70 | | | | |
| Com- | 4.77E+06 | 1.67E+06 | 1.57E+07 | 1.75E+09 |
| pound 71 | | | | |
| Com- | 2.78E+06 | 7.23E+05 | 5.53E+06 | 3.60E+09 |
| pound 72 | | | | |
| Com- | 2.93E+06 | 7.08E+06 | 1.77E+07 | — |
| pound 73 | | | | |
| Com- | 2.80E+04 | 6.66E+04 | 1.94E+05 | — |
| pound 74 | | | | |
| Com- | 1.59E+04 | 2.15E+04 | 4.51E+04 | — |
| pound 75 | | | | |
| Com- | 3.79E+04 | 7.14E+04 | 2.38E+05 | — |
| pound 76 | | | | |
| Com- | 5.38E+06 | 7.80E+06 | 3.24E+07 | — |
| pound 77 | | | | |
| Com- | 2.42E+06 | 6.65E+06 | 2.21E+07 | — |
| pound 78 | | | | |
| Com- | 7.26E+05 | 1.84E+06 | 1.65E+07 | — |
| pound 79 | | | | |
| Com- | 3.01E+06 | 2.97E+06 | 1.95E+07 | — |
| pound 80 | | | | |
| Com- | 4.91E+04 | 5.85E+04 | 2.47E+06 | — |
| pound 81 | | | | |
| Com- | 1.48E+04 | 1.70E+04 | 6.18E+04 | — |
| pound 82 | | | | |
| Com- | 1.85E+05 | 4.41E+05 | 2.14E+06 | — |
| pound 83 | | | | |
| Com- | 5.18E+04 | 8.32E+04 | 5.52E+05 | — |
| pound 84 | | | | |
| Com- | 7.47E+06 | 1.03E+07 | 6.32E+07 | — |
| pound 85 | | | | |
| Com- | 3.99E+06 | 7.58E+06 | 3.80E+07 | — |
| pound 86 | | | | |
| Com- | 1.52E+06 | 3.16E+06 | 6.61E+06 | — |
| pound 87 | | | | |
| Com- | 3.65E+05 | 9.41E+05 | 1.24E+07 | — |
| pound 88 | | | | |
| Com- | 7.66E+06 | 9.61E+06 | 3.88E+07 | — |
| pound 89 | | | | |

TABLE 5
Biological results of in vitro testing on luciferin signal via
photon measurement.

| | | | Total Liver | Total | |
|---|---|---|---|---|---|
| | Total | | Tissue | Spleen | |
| | Luciferin | | Luciferin | Tissue | |
| | Signal | | Signal | Luciferin | |
| | (RLU) | | (Photons/s) | Signal | |
| Compound | In Vitro- | In Vitro- | In Vitro- | (Photons/s) | |
| # | AML12 | B16F10 | MC38 | In Vivo-Cd1 | |
| Benchmark | 1.58E+06 | 1.90E+06 | 2.48E+06 | 1.09E+10 | 3.25E+08 |
| 1* | | | | | |
| Compound | 2.69E+04 | 8.93E+04 | — | 3.35E+06 | 5.69E+05 |
| 90 | | | | | |
| Compound | 1.94E+04 | 2.02E+05 | — | 3.65E+06 | 1.44E+06 |
| 91 | | | | | |
| Compound | 2.46E+04 | 8.03E+05 | — | 1.07E+07 | 2.05E+06 |
| 92 | | | | | |
| Compound | 3.05E+07 | 4.80E+05 | 3.69E+05 | 1.72E+06 | 2.48E+06 |
| 93 | | | | | |
| Compound | 1.41E+06 | 2.82E+06 | 1.75E+06 | 1.30E+07 | 1.22E+07 |
| 94 | | | | | |
| Compound | 1.49E+05 | 2.09E+05 | 7.12E+04 | 2.92E+06 | 2.69E+06 |
| 95 | | | | | |
| Compound | 1.49E+05 | 1.16E+05 | 1.25E+05 | 4.34E+06 | 6.35E+06 |
| 96 | | | | | |
| Compound | 2.58E+05 | 2.98E+06 | 6.42E+05 | 2.08E+08 | 1.60E+07 |
| 97 | | | | | |
| Compound | 4.38E+04 | 1.27E+05 | 7.95E+04 | | |
| 98 | | | | | |
| Compound | 1.00E+05 | 5.27E+05 | 7.66E+04 | 1.81E+09 | 2.78E+07 |
| 99 | | | | | |
| Compound | 1.43E+04 | 6.29E+04 | 3.18E+04 | 6.44E+09 | 2.92E+07 |
| 100 | | | | | |
| Compound | 9.36E+04 | 6.73E+04 | 6.90E+05 | 6.70E+08 | 1.57E+08 |
| 101 | | | | | |

*As used herein, Benchmark 1 or Benchmark compound refers to:

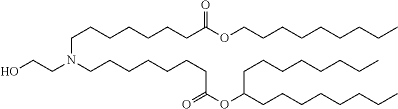 [SMILES: CCCCCCCCCOC(=O)CCCCCCCN(CCO)CCCCCCCC(=O)OC(CCCCCCCC)CCCCCCCC]

### Example 4. In Vivo Experimental

Fluc mRNA LNPs were administered to CD1 mice via the tail vein with two doses of 1.0 mg/kg at Day 0 and Day 2. Eighteen hours after second dose, bioluminescence was measured using an IVIS Spectrum imager. Mice were treated with 150 mg/kg luciferin substrate administered via intraperitoneal injection. Afterwards, mice were euthanized, and the lung, liver and spleen were harvested for ex vivo imaging. All procedures were conducted according to the approved IACUC guidelines.

Day 0: (1) Hood was set up with laptop for recording, heat lamp to express veins (with space for 1 cage), 7 1 ml tuberculin syringes with 28 g ½″ needle attached, 14 sheets of 4×4 gauze, 70% Isopropyl alcohol, scale, temporary animal holding containers, and Test Articles on wet ice. (2) Animals were transferred from R5030 into R5025 using a cart. (3) 1 cage at a time (in order of group) was transferred into the hood, placed under the heat lamp, and lid/food/water was removed. (4) Animal cages were identified. (5) Test article was pulled up into the tuberculin syringe, all air bubbles removed, and flushed into TA container to avoid animal harm and save remainders. (6) 1 gauze pad was soaked in 70% isopropyl alcohol and 1 gauze pad is placed off to the side for blood flow restriction. (7) Animal was manually restrained, and ear punched (Jackson laboratory schematic, Braintree 2 mm ear punch) and placed into temporary animal holding container to destress from initial handling. (8) Animal was mechanically restrained via tubular restrainer (waiting for nose to be within gap before fully restraining to ensure proper oxygen flow) and tail is swabbed with 70% isopropyl alcohol gauze to disinfect. (9) Test article is verified one more time with study design and animal was dosed 100 ul via left TV. (10) Thumb from tail restricting hand was placed over injection site before needle was withdrawn to avoid TA leak out from injection site. (11) Stopper on mechanical restrainer is removed while tail is still pinched off and animal is removed from mechanical restraint. (12) Finger from injection site is removed and gauze is then used to stop blood flow. (13) While pinching off wound animal is identified via temporary tail marking. (14) As soon as bleeding has stopped, animal is weighed and placed into temporary holding container. (15) Once all animals in the group had been dosed, all injection sites were observed to ensure no more bleeding had occurred and they were returned to the home cage. (16)Repeat steps 3-15 until all groups are complete and normalized before returning to R5030. Day 1: (1) Hood in R5030 was set up with laptop for recording, scale and temporary animal holding containers. (2) Animals were verified via tail marking (if number was not visible animal was restrained, verified via ear punch, and reidentified with tail marking. (3) Once verified, animal was weighed and placed in temporary holding container. (4) After all animals in the group were weighed, they were returned to their home cage. (5) Steps 2-5 were repeated until all animals were weighed. (6) Animals were then returned to their housing rack. Day 2: (1) Hood was set up with heat lamp to express veins (with space for 1 cage), 7 1 ml tuberculin syringes with 28 g ½″ needle attached, 14 sheets of 4×4 gauze, 70% Isopropyl alcohol, scale, temporary animal holding containers, Test Articles on wet ice, and Wanda was positioned outside of the hood with laptop to record body weights. (2) Animals were transferred from R5030 into R5024 using a cart. (3) 1 cage at a time (in order of group) was transferred into the hood, placed under the heat lamp, and lid/food/water was removed. (4) Test article was pulled up into the tuberculin syringe, all air bubbles removed, and flushed into TA container to avoid animal harm and save remainders. (5) 1 gauze pad was soaked in 70% isopropyl alcohol and 1 gauze pad is placed off to the side for blood flow restriction. (6) Animal was manually restrained, and ear punched (Jackson laboratory schematic, Braintree 2 mm ear punch) and placed into temporary animal holding container to destress from initial handling. (7) Animal was mechanically restrained via tubular restrainer (waiting for nose to be within gap before fully restraining to ensure proper oxygen flow) and tail is swabbed with 70% isopropyl alcohol gauze to disinfect. (8) Test article is verified one more time with study design and animal was dosed 100 ul via left TV. (9) Thumb from tail restricting hand was placed over injection site before needle was withdrawn to avoid TA leak out from injection site. (10) Stopper on mechanical restrainer is removed while tail is still pinched off and animal is removed from mechanical restraint. (11) Finger from injection site is removed and gauze is then used to stop blood flow. (12) While pinching off wound animal is identified via temporary tail marking. (13) As soon as bleeding has stopped, animal is weighed and placed into temporary holding container. (14) Once all animals in the group had been dosed, all injection sites were observed to ensure no more bleeding had occurred and they were returned to the home cage. (16)Repeat steps 3-15 until all groups are complete and normalized before returning to R5030. Day 3: (1) Hood in R5030 was set up with laptop for recording, scale and temporary animal holding containers. (2) Animals were verified via tail marking (if number was not visible animal was restrained, verified via ear punch, and reidentified with tail marking. (3) Once verified, animal was weighed and placed in temporary holding container. (4) After all animals in the group were weighed, they were returned to their home cage. (5) Steps 2-5 were repeated until all animals were weighed. (6) Animals were then placed on cart and transferred to R5033. (7) Animals were injected with 100 ul of furimazine by CRADL tech and allowed to incubate for 5 mins. (8) Once timer went off, animal was immediately euthanized. (9) Cervical dislocation was performed by tech, Wanda, or I. (10) After secondary method and animal identification was confirmed, a transverse abdominal incision was made to expose internal organs. (11) Spleen was excised first, and connective tissue was trimmed off. (12) Liver was carefully dissected away from stomach, then all dorsal connection, before excising away from the diaphragm, and removed. (13) Once liver is removed a midline cut is made through the diaphragm, midline through sternum, and ribs are broken to expose the thoracic cavity. Heart and thymus are removed to allow free access to the lungs. (14) Lungs are excised by pulling on the trachea and carefully snipping any connective tissue. (15) All Organs were laid on a black anti-luminescent/fluorescent matt in anatomical order (top to bottom) and animal order (left to right). (16) Organs were then imaged in the IVIS under auto luminescent conditions (stage height D) before organs were disposed of. (17) Steps 7-14 were repeated until all groups were complete. (18) Space was cleaned according to CRADL guidelines. The results are shown in FIG. 7, FIG. 8, and FIG. 9.

### Example 5. NHP Study Experimental 1

Male cynomolgus monkeys (Biomere, Richmond CA) were 4-6 years old and weighed 4-7 kg at the initiation of dosing. Monkeys were housed in stainless steel cages and provided with Monkey Diet 5038 daily except during times of fasting. Filtered tap water was provided to the animals ad libitum including during fasting times. Temperature of the animal room was kept between 64-84° F., humidity of 30-70%, and with a light/dark cycle of 12 hours except during designated procedures. Pre-dosing treatment with Famotidine (0.5 mg/kg), Diphenhydramine (5 mg/kg), and Dexamethasone (1 mg/kg) was performed prior to dosing of LNPs. Animals received a single intravenous IV infusion of human erythropoietin encapsulated mRNA lipid nanoparticles (hEPO mRNA LNPs, 0.3 mg/kg). LNPs were infused at 5 mL/kg/hour using an SAI 3D Programmable Syringe Pump (SAI 3D™) and a Male Luer Lock Adapter (Baxter 2C6227) extension set. Prior and after dosing, body weights and temperature were monitored and blood samples were collected pre-dose, 2, 6, 24, 48, and 168 hours. All procedures were conducted according to the approved IACUC guidelines. The results are shown in FIG. 12 and FIG. 13. Improved tolerability was observed in novel lipid formulations of mRNA.

### Example 6. NHP Study Experimental 2-Weekly Dosing of LNPs

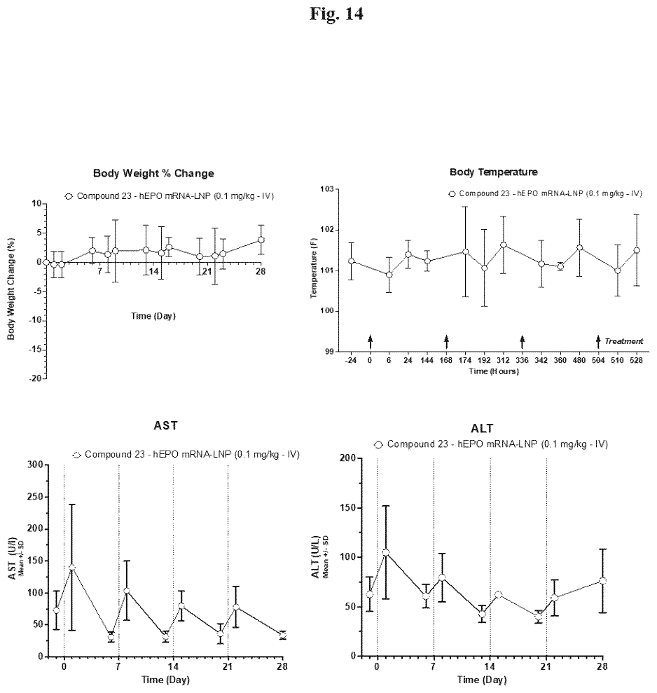

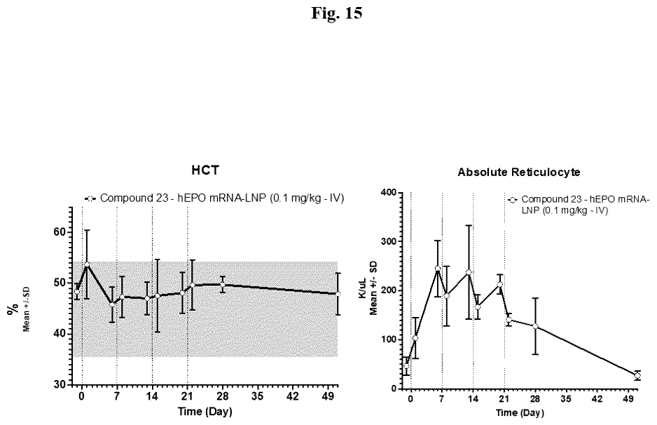

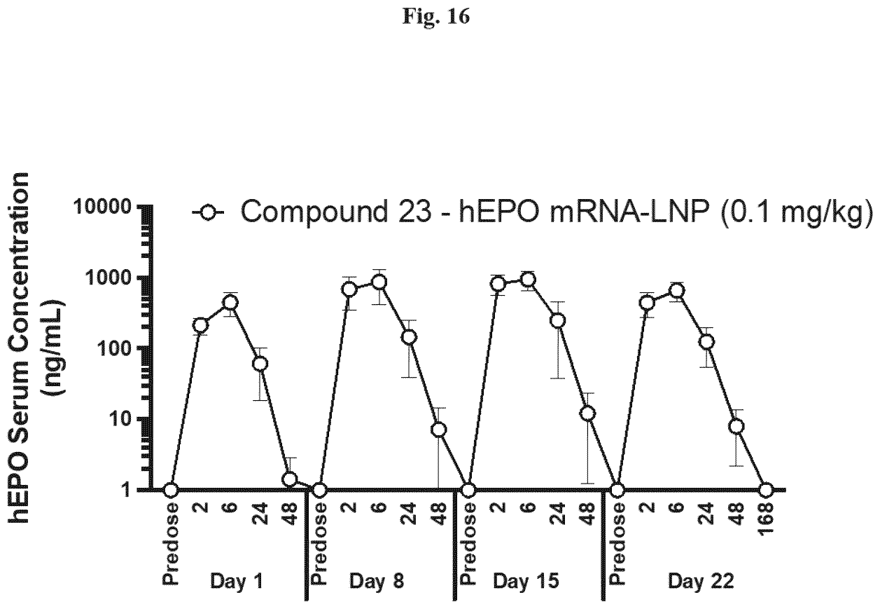

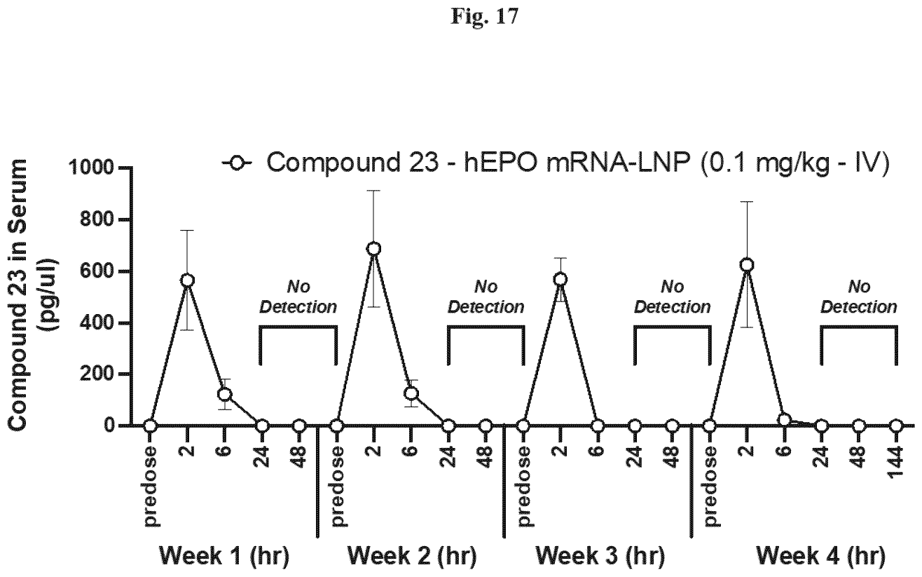

Male cynomolgus monkeys (Biomere, Richmond CA) were 4-6 years old and weighed 4-7 kg at the initiation of dosing. Monkeys were housed in stainless steel cages and provided with Monkey Diet 5038 daily except during times of fasting. Filtered tap water was provided to the animals ad libitum including during fasting times. Temperature of the animal room was kept between 64-84° F., humidity of 30-70%, and with a light/dark cycle of 12 hours except during designated procedures. Pre-dosing treatment with Famotidine (0.5 mg/kg), Diphenhydramine (5 mg/kg), and Dexamethasone (1 mg/kg) was performed prior to dosing of LNPs. Animals received weekly intravenous IV infusions of human erythropoietin encapsulated mRNA lipid nanoparticles (Compound 23, hEPO mRNA-LNP, 0.1 mg/kg). A total of four doses were administered. Prior and after dosing, body weights and temperature were monitored and blood samples were collected pre-dose, 2, 6, 24, 48, and 168 hours. All procedures were conducted according to the approved IACUC guidelines. The results are shown in FIG. 14, FIG. 15, FIG. 16, and FIG. 17. The present Example demonstrates consistent protein expression over repeated doses of encapsulated mRNA lipid nanoparticles (Compound 23, hEPO mRNA-LNP) and the clearance of Compound 23 from serum. The fluctuations of body weight, temperature, and liver blood chemistry demonstrate reasonable tolerability of Compound 23 over 4 doses. See FIG. 14. FIG. 15, among other things, demonstrates consistent generation of red blood cells over four repeated doses. FIG. 16, among other things, demonstrates that human erythropoietin is expressed consistently after each dose and is excreted into the blood, which can lead to generation of red blood cells. FIG. 17, among other things, demonstrates that Compound 23 is cleared from the blood without any observation of accumulation over repeated doses.

### Example 7. Comparative Study of LNPs Comprising Compound 23 and Benchmark 1

Multi-dose Tolerability Screening of Compound 23-LNPs: Compound 23 and Benchmark 1 were formulated using a standard process method and injected (1.0 mg/kg, IV) twice a week for four weeks into naïve Balb/c mice. NT-beta-Actin mRNA LNPs were administered IV to Balb/c mice via the tail vein for a total of eight doses (twice a week for four consecutive weeks) at a concentration of 1.0 mg/kg. Bodyweights were monitored and blood was collected pre-dose, 24 hours post first dose, 24 hours post-fourth dose, and 24 hours post-eighth dose and analyzed for cytokines, blood chemistry, and liver/spleen weights. Data analysis was performed in Graphpad Prism 10. Values are reported as mean+/−standard deviation, unless specified otherwise. Comparable bodyweights or survival rate was found among Compound 23, Benchmark 1, and the delivery buffer. N=6 per group. The results are shown in FIG. 18. The results show that subjects have comparable tolerability in mice model toward LNPs comprising Compound 23, Benchmark 1, and delivery buffer.

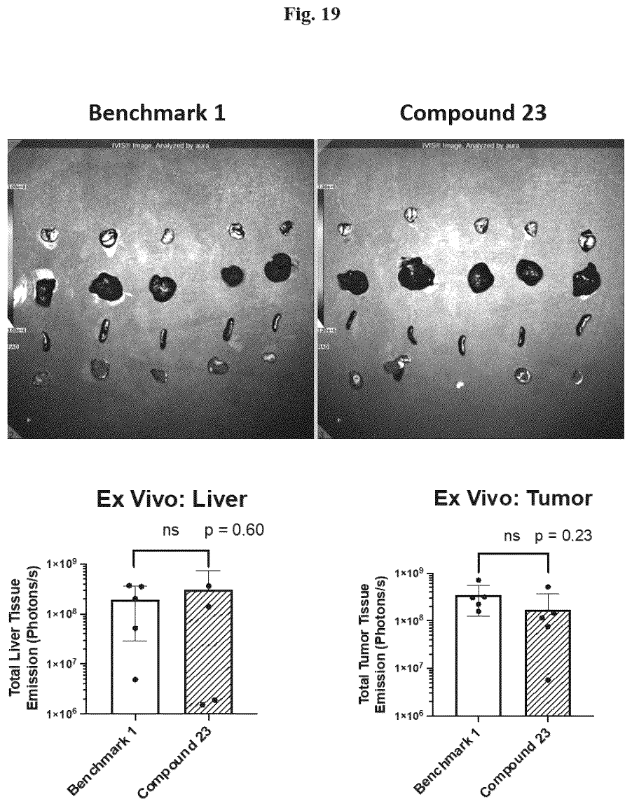

Intratumor Delivery of Compound 23 versus Benchmark 1. Albino B6 mice were inoculated with MC38.K cancer cells. Once tumors reached 250 mm³, 1.0 mg/kg of Firefly Luciferase mRNA-LNPs containing either Benchmark 1 or Compound 23 as the ionizable lipid component was dosed intratumorally. Takedown and IVIS imaging were performed 18 hours post dose. N=5 mice per group. The results are shown in FIG. 19. These results show that Compound 23 demonstrated comparable delivery performance as that of Benchmark 1.

### Example 8. Comparative Study-Effects of Fluorination on Transfection Efficiency

Transfection efficiency was compared between three pairs of compounds, Compound 54 v. Compound 58, Compound 68 v. Compound 67, and Compound 37 v. Compound 61. For each pair, the compounds only differ on one fluoro-substitution at the end of an alkyl chain. For example, the structures of Compound 54 and Compound 58 are shown below:

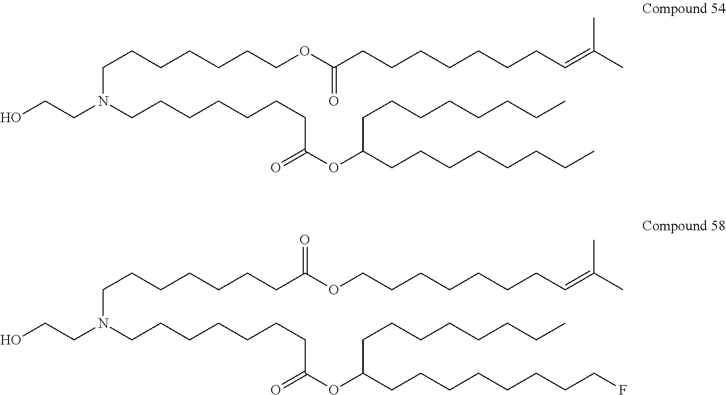 [SMILES: CCCCCCCCC(CCCCCCCC)OC(=O)CCCCCCCN(CCO)CCCCCCCOC(=O)CCCCCCCC=C(C)C ; CCCCCCCCC(CCCCCCCCF)OC(=O)CCCCCCCN(CCO)CCCCCCCC(=O)OCCCCCCCC=C(C)C]

In vitro and in vivo comparison of Firefly Luciferase expression data of fluoro v. non-fluoro compounds were obtained according to procedure similar to those in Example 3 and Example 7. The results are shown in FIG. 20. Statistically significant difference in transfection efficiency was observed between Compound 54 v. Compound 58 and Compound 68 v. Compound 67 in hepatocytes.

Example 9. Synthesis of heptadecan-9-yl 8-[(2-hydroxyethyl)({6-[(9-methyldecyl)oxy]-6-oxohexyl})amino]octanoate

This example illustrates synthesis of:

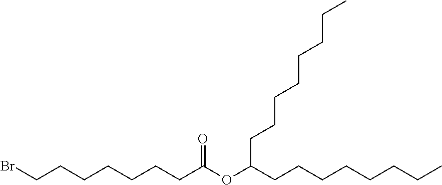 [SMILES: CCCCCCCCC(CCCCCCCC)OC(=O)CCCCCCCBr]

heptadecan-9-yl 8-bromooctanoate; chemical Formula: C₂₅H₄₉BrO₂; molecular Weight: 461.57.

To a stirred solution of 8-bromooctanoic acid (25 g, 112 mmol, 1.0 eq) in dichloromethane (1 L), was added 4-(dimethylamino)pyridin-1-ium (13.8 g, 112 mmol, 1.0 eq) and {3-[cyano(ethyl)amino]propyl}dimethylazanium chloride (64.4 g, 336 mmol, 3.0 eq). The reaction mixture was stirred for 15 min and added heptadecan-9-ol (25.9 g, 101 mmol, 0.9 eq) to it. The reaction mixture was stirred at room temperature (r. t.) for 48 h. The progress of reaction was monitored by TLC (SM was consumed). Water (250 mL) was added to the reaction mixture, and extracted with DCM (3×250 mL). The resulting organic layer was dried over Na2SO4, and concentrated under reduce pressure, and the crude was purified by flash column chromatography (SiO2: 0-2% Ethyl acetate in Hexanes) to give heptadecan-9-yl 8-bromooctanoate (31.65 g, Yield=61.19%) as a pale yellow liquid. 1H-NMR (400 MHz, CDCl3-d3): δ 4.88-4.84 (m, 1H), 3.54-3.38 (m, 2H), 2.28 (t, J=7.6 Hz, 2H), 1.88-1.76 (m, 2H), 1.64-1.58 (m, 2H), 1.50-1.49 (m, 4H), 1.44-1.41 (m, 2H), 1.36-1.29 (m, 4H), 1.25 (br, 24H), 0.87 (t, J=7.2 Hz, 6H).

Example 10. Synthesis of heptadecan-9-yl 8-[(2-hydroxyethyl)amino]octanoate

This example illustrates synthesis of:

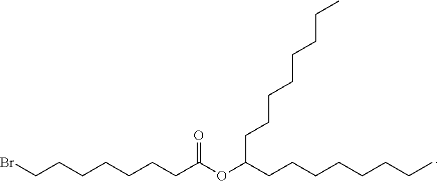 [SMILES: CCCCCCCCC(CCCCCCCC)OC(=O)CCCCCCCBr]

To a stirred solution of 8-bromooctanoic acid (25 g, 112 mmol, 1.0 eq) in dichloromethane (1 L), was added 4-(dimethylamino)pyridin-1-ium (13.8 g, 112 mmol, 1.0 eq) and {3-[cyano(ethyl)amino]propyl}dimethylazanium chloride (64.4 g, 336 mmol, 3.0 eq). The reaction mixture was stirred for 15 min and added heptadecan-9-ol (25.9 g, 101 mmol, 0.9 eq) to it. The reaction mixture was stirred at room temperature (r.t.) for 48 hours (h). The progress of reaction was monitored by TLC (SM was consumed). Water (250 mL) was added to the reaction mixture, and extracted with DCM (3×250 mL). The resulting organic layer was dried over Na₂SO₄, and concentrated under reduce pressure, and the crude was purified by flash column chromatography (SiO₂: 0-2% Ethyl acetate in Hexanes) to give heptadecan-9-yl 8-bromooctanoate (31.65 g, Yield=61.19%) as a pale yellow liquid. ¹H-NMR (400 MHz, CDCl₃-d₃): δ 4.88-4.84 (m, 1H), 3.54-3.38 (m, 2H), 2.28 (t, J=7.6 Hz, 2H), 1.88-1.76 (m, 2H), 1.64-1.58 (m, 2H), 1.50-1.49 (m, 4H), 1.44-1.41 (m, 2H), 1.36-1.29 (m, 4H), 1.25 (br, 24H), 0.87 (t, J=7.2 Hz, 6H).
Example 11. Synthesis of heptadecan-9-yl 8-[(2-hydroxyethyl)amino]octanoate

This example illustrates synthesis of:

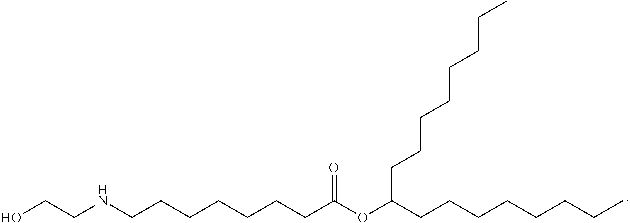 [SMILES: CCCCCCCCC(CCCCCCCC)OC(=O)CCCCCCCNCCO]

To a stirred solution of 2-aminoethan-1-ol (794 mg, 13 mmol, 1.0 eq) in acetonitrile (50 mL), was added heptadecan-9-yl 8-bromooctanoate (6.0 g, 13 mmol, 1.0 eq) and ethylbis(propan-2-yl)amine (2.72 mL, 15.6 mmol, 1.2 eq) to it. The reaction was stirred at 55° C. for 4 days. The progress of reaction was monitored by ELSD/TLC (87% product form in reaction mixture by ELSD). The reaction mixture was concentrated under reduced pressure and the crude was purified by flash column chromatography (SiO₂: 0-12% Methanol in Dichloromethane) to give heptadecan-9-yl 8-[(2-hydroxyethyl)amino]octanoate (2.1 g. Yield=36%) as a brown gummy liquid. ELSD analysis: Purity 97.47%, Calculated C₂₇H₅₆NO₃, [M+H]=442.43, Observed=442.35 (m/z, M+H⁺).
Example 12. Synthesis of 8-(benzyloxy)-1-octanol

This example illustrates synthesis of:

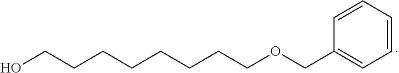 [SMILES: OCCCCCCCCOCc1ccccc1]

To a stirred solution of 1,8-octanediol (10 g, 68.4 mmol, 1.0 eq) in dimethylformamide (100 mL) was added of sodium hydride (60% dispersion in mineral oil) (2.36 g, 103 mmol, 1.5 eq) at 0° C. and stirred for 30 min at same temperature. Then (bromomethyl)benzene (11.7 g, 68.4 mmol, 1.0 eq) was added to RM and the reaction mixture was stirred at r.t. for 16 h. The progress of reaction was monitored by TLC, after completion of the reaction the reaction mixture was quenched with ice-cooled water (250 ml) and extracted with ethyl acetate (50×3 mL). The resulting organic layer was dried over Na₂SO₄, and concentrated under reduce pressure, and the crude was purified by flash column chromatography (SiO₂: 0-15% Ethyl acetate in Hexane) to give 8-(benzyloxy)-1-octanol (7.4 g. Yield=45.78%) as a white solid. ¹H NMR (400 MHz, CDCl₃-d3): δ 7.36-7.27 (m, 5H), 4.50 (s, 2H), 3.63 (t, J=6.8 Hz, 2H), 3.46 (t, J=6.8 Hz, 2H), 1.64-1.52 (m, 3H), 1.41-1.32 (m, 9H).
Example 13. Synthesis of 8-(benzyloxy)octanal

This example illustrates synthesis of:

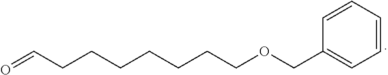 [SMILES: O=CCCCCCCCOCc1ccccc1]

To a stirred solution of 8-(benzyloxy)-1-octanol (7.4 g, 31.3 mmol, 1.0 eq) in dichloromethane (250 mL) was add pyridinium chlorochromate (10.1 g, 47 mmol, 1.5 eq) at 0° C. and the reaction mixture was stirred at r.t. for 2 h. The progress of reaction was monitored by TLC, the reaction mixture was diluted with pentane (500 ml) and stirred for 30 min. After 30 min, mixture was filtered through celite bed and washed with pentane (3×250 ml). The organic layer was concentrated under reduced pressure. The crude was purified by flash column chromatography (SiO₂: 0-25% ethylacetate in hexane) to give 8-(benzyloxy)octanal (7 g, Yield=95.1%) as a colorless liquid. ¹H NMR (400 MHz, CDCL₃-d3): δ 9.75 (m, 1H), 7.36-7.27 (m, 5H), 4.49 (s, 2H), 3.46 (t, J=6.4 Hz, 2H), 2.43-2.39 (m, 2H), 1.66-1.57 (m, 4H), 1.41-1.25 (m, 6H).
Example 14. Synthesis of decyl benzyl ether

This example illustrates synthesis of:

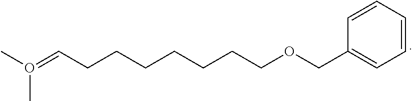

To a stirred solution of triphenyl(propan-2-yl)phosphanium bromide (9.04 g, 23.5 mmol, 1.1 eq) in tetrahydrofuran (100 mL) was added lithium 1-butanide (2.05 g, 32 mmol, 1.5 eq) dropwise at −78° C. and the reaction mixture was stirred at room temperature for 1 hr, then the mixture was cooled again to −78° C. and then 8-(benzyloxy)octanal (5 g, 21.3 mmol, 1.0 eq) (dissolved in 20 mL THF) was added dropwise and the reaction mixture was stirred at room temperature for 16 h. The progress of reaction was monitored by TLC. The reaction mixture was quenched with aq. ammonium chloride solution and extracted with ethyl acetate (3×250 mL). The organic layer was collected and dried over sodium sulphate, filtered and concentrated under vacuum to give the crude mixture. The crude product was purified by combi-flash chromatography (SiO₂: 0-15% Ethyl acetate in Hexane), to give {[(9-methyldec-8-en-1-yl)oxy]methyl}benzene (3.5 g, Yield=62.99%) as a colorless liquid. ¹H NMR (400 MHz, CDCl₃-d3): δ 7.34-7.27 (m, 5H), 5.12-5.09 (m, 1H), 4.50 (s, 2H), 3.46 (t. J=6.0 Hz, 2H), 1.96-1.94 (m, 2H), 1.68-1.59 (s, 6H), 1.37-1.32 (m, 9H).
Example 15. Synthesis of 9-methyl-1-decanol

This example illustrates synthesis of: To a stirred solution of 10-(benzyloxy)-2-methyl-2-decene (3.5 g, 13.4 mmol, 1.0 eq) in tetrahydrofuran (50 mL) and methanol (50 mL) was degassed with nitrogen for 15 min, then Pd/C, 10%, 50% wet (3.5 g, w/w) was added and stirred at r.t. for 48 h under Hydrogen atmosphere. TLC showed consumption of starting material and formation of a new spot. The reaction mixture was filtered through celite bed and washed with mixture of MeOH:THF (3×200 ml). The filtrate was collected and concentrate to get the crude. The crude was purified through combi-flash chromatography (SiO₂: 0-20% Ethyl acetate in Hexane), to get 9-methyl-1-decanol (2 g, Yield=86.36%) as colorless liquid. ¹H NMR (400 MHz, DMSO-d6): δ 4.30 (t, J=4.8 Hz, 1H), 3.38-3.31 (m, 2H), 1.52-1.46 (m, 1H), 1.40-1.37 (m, 2H), 1.24 (bs, 10H), 1.13-1.12 (m, 2H), 0.84 (d, J=6.4 Hz, 6H).

Example 16. Synthesis of 9-methyldecyl 6-bromohexanoate

This example illustrates synthesis of:

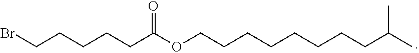 [SMILES: CC(C)CCCCCCCCOC(=O)CCCCCBr]

To a stirred solution of 6-bromohexanoic acid (2.49 g, 12.8 mmol, 1.1 eq) in dichloromethane (166 mL), was added {3-[cyano(ethyl)amino]propyl}dimethylazanium chloride (4.45 g, 23.2 mmol, 2 eq) and 4-(dimethylamino)pyridin-1-ium (1.43 g, 11.6 mmol, 1.0 eq) to it. The reaction stirred for 15 min then 9-methyldecan-1-ol (2 g, 11.6 mmol, 1.0 eq) was added. The reaction mixture was stirred at r.t. for 48 h. The progress of reaction was monitored by TLC (SM was consumed). Water (250 mL) was added to the reaction mixture and extracted with DCM (3×250 mL). The resulting organic layer was dried over Na₂SO₄ and concentrated under reduce pressure to get a crude. The crude was purified by flash column chromatography (SiO2: 0-2% Ethyl acetate in Hexanes) to give 9-methyldecyl 6-bromohexanoate (2.0 g, Yield=49.32%) as a colorless liquid. ¹H NMR (400 MHz, CDCl₃-d3): δ 4.05 (t, J=6.4 Hz, 2H), 3.55-3.39 (m, 2H), 2.31 (t, J=7.6 Hz, 2H), 1.87-1.77 (m, 2H), 1.67 (m, 4H), 1.52-1.45 (m, 3H), 1.30-1.25 (m, 10H), 1.15-1.13 (m, 2H), 0.86 (d, J=6.4 Hz, 6H).
Example 17. Synthesis of heptadecan-9-yl 8-[(2-hydroxyethyl) ({6-[(9-methyldecyl)oxy]-6-oxohexyl})amino]octanoate

This example illustrates synthesis of:

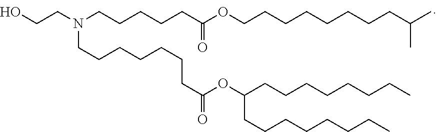 [SMILES: CCCCCCCCC(CCCCCCCC)OC(=O)CCCCCCCN(CCO)CCCCCC(=O)OCCCCCCCCC(C)C]

To a stirred solution of 9-methyldecyl 6-bromohexanoate (633 mg, 1.81 mmol, 1.0 eq) in acetonitrile (10 mL), was added N-ethylbis(isopropyl)amine (702 mg, 5.43 mmol, 3.0 eq) and heptadecan-9-yl 8-[(2-hydroxyethyl)amino]octanoate (0.8 g, 1.81 mmol, 1.0 eq). The reaction was stirred at 95° C. for 16 h. The progress of reaction was monitored by ELSD/TLC ELSD/TLC (81% product form in reaction mixture by ELSD). The reaction mixture was concentrated under reduced pressure and the crude was purified by flash column chromatography (SiO2: 0-8% Methanol in Dichloromethane) to give heptadecan-9-yl 8-[(2-hydroxyethyl)({6-[(9-methyldecyl)oxy]-6-oxohexyl})amino]octanoate (335 mg, Yield=26.05%) as a colorless liquid. Compound was dissolved in dichloromethane (10 ml), was added 5 eq of anhydrous K2CO3 under N2 atmosphere and stirred reaction mixture Vigorously 2-3 hr then reaction mixture filtered through Hydrophobic PTFE Syringe Filter, 25 mm*0.45 μm by glass syringe to get desired compound. 1H-NMR (400 MHz, CDCl3-d3): δ 4.87-4.82 (m, 1H), 4.05 (t, J=6.8 Hz, 2H), 3.52 (t, J=5.2 Hz, 2H), 2.56 (t, J=5.2 Hz, 2H), 2.46-2.41 (m, 4H), 2.31-2.25 (m, 4H), 1.67-1.57 (m, 7H), 1.55-1.38 (m, 9H), 1.38-1.25 (m, 42H), 1.51-1.11 (m, 2H), 0.89-0.85 (m, 12H). ELSD analysis: Purity 98.98%, Calculated C44H88NO5, [M+H]=710.67, Observed=710.50 (m/z, M+H+).
Example 18. Synthesis of heptadecan-9-yl 8-((2-hydroxyethyl)(8-((9-methyldecyl)oxy)-8-oxooctyl)amino)octanoate

This example illustrates synthesis of:

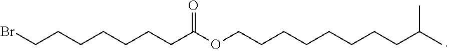 [SMILES: CC(C)CCCCCCCCOC(=O)CCCCCCCBr]

To a stirred solution of 8-bromooctanoic acid (8.29 g, 37.1 mmol, 2.0 eq) in dichloromethane (166 mL), was added {3-[cyano(ethyl)amino]propyl}dimethylazanium chloride (14.2 g, 74.3 mmol, 4 eq.) and 4-(dimethylamino)pyridin-1-ium (4.58 g, 37.1 mmol, 2 eq.) to it. The reaction was stirred for 15 min then 9-methyl-1-decanol (3.2 g, 18.6 mmol, 1.0 eq) was added. The reaction mixture was stirred at r.t. for 48 h. The progress of reaction was monitored by TLC (SM was consumed). Water (250 mL) was added to the reaction mixture and extracted with DCM (3×250 mL). The resulting organic layer was dried over Na₂SO₄ and concentrated under reduce pressure to get a crude. The crude was purified by flash column chromatography (SiO2: 0-2% Ethyl acetate in Hexanes) to give 9-methyldecyl 8-bromooctanoate (3.8 g Yield=54.22%) as a colorless liquid. ¹H NMR (400 MHz, CDCl3-d3): δ 4.05 (t, J=6.4 Hz, 2H), 3.55-3.39 (m, 2H), 2.29 (t, J=7.6 Hz, 2H), 1.89-1.82 (m, 2H), 1.64-1.54 (m, 4H), 1.49-1.42 (m, 3H), 1.36-1.28 (m, 14H), 1.25-1.15 (m, 2H), 0.86 (d, J=6.8 Hz, 6H).
Example 19. Synthesis of heptadecan-9-yl 8-((2-hydroxyethyl)(8-((9-methyldecyl)oxy)-8-oxooctyl)amino)octanoate

This example illustrates synthesis of:

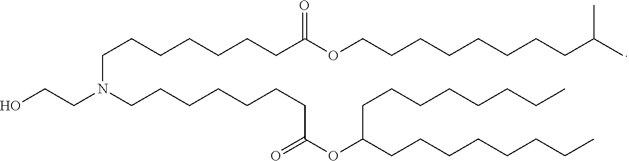 [SMILES: CCCCCCCCC(CCCCCCCC)OC(=O)CCCCCCCN(CCO)CCCCCCCC(=O)OCCCCCCCCC(C)C]

To a stirred solution of heptadecan-9-yl 8-[(2-hydroxyethyl)amino]octanoate (1.1 g, 2.49 mmol, 1.0 eq) in acetonitrile (5 mL), was added N-ethylbis(isopropyl)amine (0.96 g, 7.47 mmol, 3 eq) and 9-methyldecyl 8-bromooctanoate (1.03 g, 2.74 mmol, 1.1 eq) to it. The reaction was stirred at 90° C. for 16 h. The progress of reaction was monitored by ELSD/TLC (91% product form in reaction mixture by ELSD). The reaction mixture was concentrated under reduced pressure and the crude was purified by flash column chromatography (SiO₂: 0-8% Methanol in Dichloromethane), to give heptadecan-9-yl 8-[(2-hydroxyethyl)({8-[(9-methyldecyl)oxy]-8-oxooctyl})amino]octanoate (0.7 g, Yield=38.19%) as a pale-yellow liquid. ¹H-NMR (400 MHz, CDCl₃-d3): δ 4.88-4.82 (m, 1H), 4.05 (t, J=6.8 Hz, 2H), 3.69 (bs, 2H), 2.77-2.65 (br, 4H), 2.30-2.27 (m, 4H), 1.64-1.56 (m, 8H), 1.54-1.47 (m, 6H), 1.32-1.25 (m, 49H), 1.15-1.13 (m, 2H), 0.89-0.85 (m, 12H). ELSD analysis: Purity 99.86%, Calculated C₄₆H₉₂NO₅, [M+H]=738.70, Observed=738.55 (m/z, M+H⁺).
Example 20. Synthesis of 1-octylnonyl 8-({2-[4-(dimethylamino)butyroxy]ethyl}[7-(9-methyldecyloxycarbonyl)heptyl]amino)octanoate

This example illustrates synthesis of:

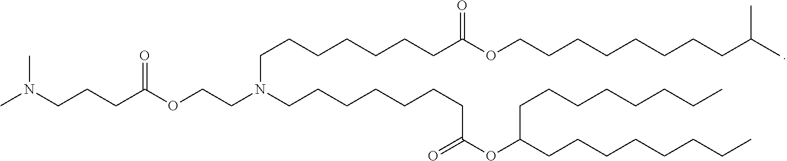 [SMILES: CCCCCCCCC(CCCCCCCC)OC(=O)CCCCCCCN(CCCCCCCC(=O)OCCCCCCCCC(C)C)CCOC(=O)CCCN(C)C]

To a stirred solution of 4-(dimethylamino)butyric acid (355 mg, 4 eq., 2.71 mmol) in dichloromethane (10 ml) was added 3-[cyano(ethyl)amino]propyl}dimethylazanium chloride (519 mg, 2.71 mmol, 4 eq) and 4-(dimethylamino)pyridin-1-ium (82.7 mg, 677 μmol, 1 eq) and stirred for 15 min, then 1-octylnonyl 8-{(2-hydroxyethyl)[7-(9-methyldecyl oxycarbonyl)heptyl]amino}octanoate (0.5 g, 677 μmol, 1.0 eq) was added. The reaction mixture was stirred at r.t. for 16 h. The progress of reaction was monitored by ELSD/TLC (58.22% product form in reaction mixture with 39% DMAP by ELSD). The reaction mixture was quenched with brine solution and extracted with DCM (2×100 mL). The combined organic layer was dried over sodium sulphate, filtered and concentrated to get the crude mixture. The crude was diluted with pentane and washed with acetonitrile (3×100 ml). Pentane layer was evaporated and distilled (temp. below 30° C.) to get 1-octylnonyl 8-({2-[4-(dimethylamino)butyroxy]ethyl}[7-(9-methyldecyloxycarbonyl)heptyl]amino)octanoate (340 mg, Yield=58.96%) as colorless liquid. 1H-NMR (400 MHz, CDCl₃)-δ 4.87-4.84 (m, 1H), 4.10 (t, J=6.4 Hz, 2H), 4.05 (t, J=6.8 Hz, 2H), 2.67-2.64 (m, 2H), 2.42 (t, J=7.6 Hz, 4H), 2.35-2.31 (m, 2H), 2.30-2.25 (m, 6H), 2.20 (s, 6H), 1.81-1.74 (m, 2H), 1.62-1.56 (m, 5H), 1.52-1.46 (m, 4H), 1.44-1.37 (m, 4H), 1.30-1.25 (m, 48H), 1.15-1.11 (m, 2H), 0.89-0.85 (m, 12H). ELSD analysis: Purity 99.49%, Calculated C₅₂H₁₀₃N₂O₆, [M+H]=851.78, Observed=851.60 (m/z, M+H⁺).
Example 21. Synthesis of 2-octyldecanoic acid

This example illustrates synthesis of:

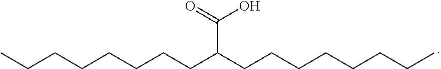 [SMILES: CCCCCCCCC(CCCCCCCC)C(=O)O]

To a stirred solution of decanoic acid (20 g, 116 mmol, 1.1 eq) in tetrahydrofuran (267 ml), was added sodium hydride (5.11 g, 128 mmol, 1.1 eq) at 0° C. and stirred for 30 minutes, lithium bis(isopropyl)azanide (14.9 g, 139 mmol, 1.2 eq) was added to the reaction mixture at the −50° C. temperature, and the reaction mixture was stirred at room temperature for 30 minutes. Then 1-iodooctane (33.5 g, 139 mmol, 1.2 eq) was added. The reaction mixture was stirred at 45° C. for 16 h. The progress of reaction was monitored by TLC (SM was consumed). The reaction mixture was poured into a mixture of a 1 mol/L aq. HCl (250 mL) and extracted with ethyl acetate (3×250 mL). The resulting organic layer was dried over Na₂SO₄ and concentrated under reduce pressure to get crude. The crude was purified by flash column chromatography (SiO₂: 0-2% Ethyl acetate in Hexanes), to give 2-octyldecanoic acid (10 g, Yield=30.28%) as a white solid. 1H-NMR (400 MHz, DMSO-d6): δ 12.02 (bs, 1H), 2.20-2.13 (m, 1H), 1.48-1.43 (m, 2H), 1.37-1.34 (m, 2H), 1.32-1.22 (m, 24H), 0.86-0.85 (m, 6H).
Example 22. Synthesis of 7-bromoheptyl 2-octyldecanoate

This example illustrates synthesis of:

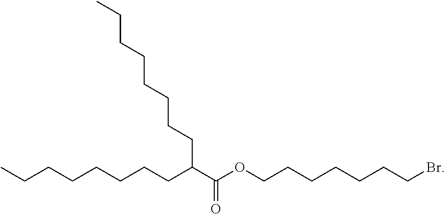 [SMILES: CCCCCCCCC(CCCCCCCC)C(=O)OCCCCCCCBr]

To a stirred solution of 2-octyldecanoic acid (4 g, 14.1 mmol, 1.0 eq) in dichloromethane (48 mL, 750 mmol), was added {3-[cyano(ethyl)amino]propyl}dimethylazanium chloride (10.8 g, 56.2 mmol, 4 eq.,) and 4-(dimethylamino)pyridin-1-ium (3.46 g, 28.1 mmol, 2 eq.,) to it. The reaction mixture was stirred for 15 min then 7-bromo-1-heptanol (2.74 g, 14.1 mmol, 1.0 eq) was added. The reaction mixture was stirred at r.t. for 48 h. The progress of reaction was monitored by TLC (SM was consumed). Water (250 ml) was added to the reaction mixture and extracted with DCM (3×250 mL). The resulting organic layer was dried over Na₂SO₄, and concentrated under reduce pressure, and the crude was purified by flash column chromatography (SiO₂: 0-2% Ethyl acetate in Hexanes) to give a 7-bromoheptyl 2-octyldecanoate (3 g, Yield=46.23%) as a colorless liquid. 1H-NMR (400 MHz, CDCL₃-d3): δ 4.08-4.04 (m, 2H), 3.53-3.38 (m, 2H), 2.39-2.28 (m, 2H), 1.87-1.75 (m, 2H), 1.64-1.55 (m, 3H), 1.45-1.39 (m, 3H), 1.36-1.34 (m, 3H), 1.25 (bs, 25H), 0.89-0.85 (m, 6H).
Example 23. Synthesis of 7-(2-hydroxyethylamino)heptyl 2-octyldecanoate

This example illustrates synthesis of:

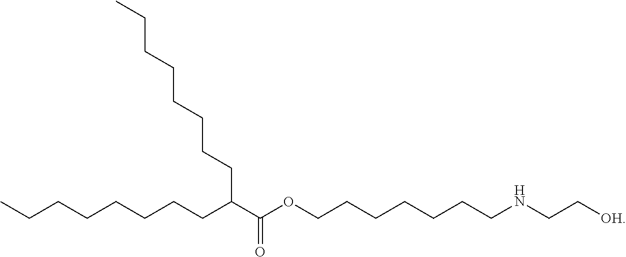 [SMILES: CCCCCCCCC(CCCCCCCC)C(=O)OCCCCCCCNCCO]

To a stirred solution of 2-aminoethanol (393 μL, 6.5 mmol, 1.0 eq) in acetonitrile (5 mL), was added 7-bromoheptyl 2-octyldecanoate (3 g, 6.5 mmol, 1.0 eq) and N-ethylbis(isopropyl)amine (3.38 mL, 19.5 mmol, 3 eq) to it. The reaction was stirred at 55° C. for 72 h. The progress of reaction was monitored by TLC/ELSD (22% product form in reaction mixture by ELSD). The reaction mixture was concentrated under reduced pressure and the crude was purified by flash column chromatography (SiO₂: 0-12% Methanol in Dichloromethane), to give 7-(2-hydroxyethylamino)heptyl 2-octyldecanoate (0.6 g, Yield=20.19%) as brown liquid. ¹H-NMR (400 MHz, CDCl₃-d3): δ 4.02 (t, J=6.8 Hz, 2H), 3.82 (t, J=4.8 Hz, 2H), 2.97-2.94 (m, 2H), 2.80 (t, J=3.6 Hz, 2H), 2.28-2.25 (m, 1H), 1.67 (bs, 2H), 1.58-1.51 (m, 4H), 1.41-1.25 (32H), 0.85-0.85 (m, 6H). ELSD analysis: Purity 99.88%, Calculated C₂₇H₅₆NO₃, [M+H]=442.43, Observed=442.40 (m/z, M+H⁺).
Example 24. Synthesis of 7-((2-hydroxyethyl)(8-((9-methyldecyl)oxy)-8-oxooctyl)amino)heptyl 2-octyldecanoate

This example illustrates synthesis of

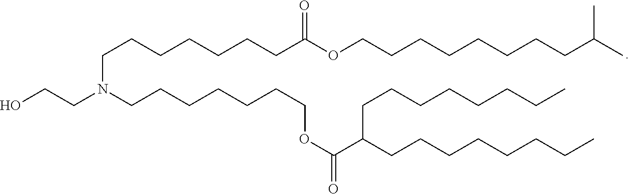 [SMILES: CCCCCCCCC(CCCCCCCC)C(=O)OCCCCCCCN(CCO)CCCCCCCC(=O)OCCCCCCCCC(C)C]

To a stirred solution of 7-(2-hydroxyethylamino)heptyl 2-octyldecanoate (0.6 g, 1.36 mmol, 1.0 eq) in acetonitrile (6 mL), was added N-ethylbis(isopropyl)amine (593 μL, 3.4 mmol, 2.5 eq) and 9-methyldecyl 8-bromooctanoate (513 mg, 1.36 mmol, 1.0 eq) to it. The reaction was stirred at 95° C. for 16 h. The progress of reaction was monitored by ELSD/TLC (31% product form in reaction mixture by ELSD). The reaction mixture was concentrated under reduced pressure and the crude was purified by flash column chromatography (SiO₂: 0-8% Methanol in Dichloromethane), to give 7-{(2-hydroxyethyl)[7-(9-methyldecyloxycarbonyl)heptyl]amino}heptyl 2-octyldecanoate (140 mg, Yield=13.6%) as a colorless liquid. ¹H-NMR (400 MHz, CDCl₃-d3): δ 4.17-4.03 (m, 4H), 3.66 (br, 2H), 2.73-2.69 (br, 2H), 2.45-2.43 (br, 6H), 2.33-2.26 (m, 3H), 1.68-1.51 (m, 12H), 1.41-1.39 (m, 3H), 1.32-1.24 (m, 49H), 1.17-1.12 (m, 2H), 0.89-0.85 (m, 12H). ELSD analysis: Purity 98.21%, Calculated C₄₆H₉₂NO₅, [M+H]=738.70, Observed=738.70 (m/z, M+H⁺).
Example 25. Synthesis of (2-bromoethoxy)(tert-butyl)bis(methyl)silane

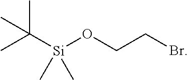

This example illustrates synthesis of: To a stirred solution of 2-bromoethanol (10 g, 80 mmol, 1.0 eq.) in dichloromethane (50 mL), imidazole (21.8 g, 320 mmol, 4 eq.) and (tert-butyl) (chloro)bis(methyl)silane (24.1 g, 160 mmol, 2 eq.) was added and reaction mixture was stirred at 25° C. for 16 h. Reaction mixture was quenched with brine solution (100 mL) and extracted with ethyl acetate (3×100 mL). The organic layer was dried over sodium sulphate, filtered, and concentrated under reduced pressure. The crude was purified by flash column chromatography (silica gel, 0-2% gradient of ethyl acetate in hexane) to obtain (2-bromoethoxy)(tert-butyl)bis(methyl)silane (15 g, Yield=78.35%) as a colorless liquid. ¹H NMR (400 MHz, CDCl₃-d3): δ 3.89 (t, J=6.4 Hz, 2H), 3.40 (t, J=6.4 Hz, 2H), 0.89 (s, 9H), 0.08 (s, 6H).

Example 26. Synthesis of methyl 4-(methylamino)butyrate

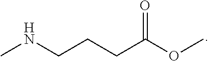

This example illustrates synthesis of: To a stirred solution of 4-(methylamino)butyric acid (5 g, 42.7 mmol) in methanol (50 mL, 1.23 mol) was added thionyl dichloride (6.09 g, 1.2 eq., 51.2 mmol) at 0° C. After complete the addition, reaction mixture stirred at room temperature for 16 h. The progress of reaction was monitored by TLC and ELSD. The reaction mixture was concentrated under reduced pressure obtain methyl 4-(methylamino)butyrate (3.5 g, Yield=62.51%) as a colorless liquid. ¹H NMR (400 MHz, MeOD): δ 3.68 (s, 3H), 3.05 (t, J=6.8 Hz, 2H), 2.70 (s, 3H), 2.49 (t, J=6.4 Hz, 2H), 1.97 (t, J=6.4 Hz, 2H).

Example 27. Synthesis of methyl 4-({2-[(tert-butyl)bis(methyl)siloxy]ethyl}-N-methylamino)butyrate

This example illustrates synthesis of:

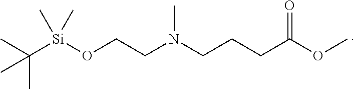 [SMILES: COC(=O)CCCN(C)CCO[Si](C)(C)C(C)(C)C]

To a solution of methyl 4-(methylamino)butyrate (2 g, 15.2 mmol, 1 equiv) in CH₃CN (20 mL), was added potassium carbonate (6.32 g, 45.7 mmol, 3 equiv) and the reaction mixture was stirred at 50° C. for 30 minutes. Then, (2-bromoethoxy)(tert-butyl)bis(methyl)silane (4.74 g, 19.8 mmol, 1.3 equiv) was added and the reaction mixture was stirred at 85° C. for 5 h. The progress of reaction was monitored by TLC and the reaction mixture was concentrated under reduced pressure. The crude was purified by flash column chromatography (silica gel, 0-13% gradient of ethyl acetate in hexane) to obtain methyl 4-({2-[(tert-butyl)bis(methyl)siloxy]ethyl}-N-methylamino)butyrate as colorless oil (1.1 g, Yield=24.9%). ELSD analysis: Purity 99.23%, Calculated for C₁₄H₃₂NO₃Si, [M+H]=290.22, Observed=290.20 (m/z, M+H⁺).
Example 28. Synthesis of 4-((2-((tert-butyldimethylsilyl)oxy)ethyl)(methyl)amino)butanoic acid

This example illustrates synthesis of:

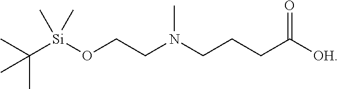 [SMILES: CN(CCCC(=O)O)CCO[Si](C)(C)C(C)(C)C]

A solution of methyl 4-({2-[(tert-butyl)bis(methyl)siloxy]ethyl}-N-methylamino)butyrate (1 g, 3.45 mmol) in a mixture of THF and water (10 mL, 1:1 v/v) was added lithium hydroxide monohydrate (580 mg, 13.8 mmol, 4 eq.) at room temperature. The reaction mixture was then stirred at room temperature for 90 minutes. After completion of the reaction (monitored by TLC), the reaction mixture was neutralized with 1M citric acid. Then, the resulting mixture was extracted with DCM (3×15 mL). The combined organic layer was dried over sodium sulfate and concentrated under reduced pressure to obtain 4-((2-((tert-butyldimethylsilyl)oxy)ethyl) (methyl)amino)butanoic acid (610 mg, Yield=84%) as sticky solid. ¹H NMR (400 MHz, CDCl₃-d3): δ 4.99 (t, J=5.2 Hz, 2H), 3.24-3.18 (m, 4H), 3.03 (s, 3H), 2.44-2.41 (m, 2H), 1.94-1.88 (m, 2H), 0.93 (s, 9H), 0.14 (s, 6H). ELSD analysis: Purity 95.51%, Calculated for C₁₃H₃₀NO₃Si, [M+H]=276.19, Observed=276.05 (m/z, M+H⁺).
Example 29. Synthesis of 1-octylnonyl 8-({2-[4-({2-[(tert-butyl)bis(methyl)siloxy]ethyl}-N-methylamino)butyroxy]ethyl}[7-(9-methyldecyloxycarbonyl)heptyl]amino)octanoate

This example illustrates synthesis of:

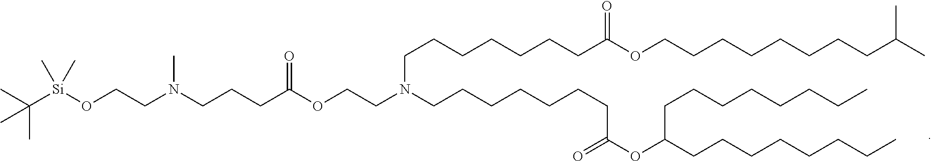 [SMILES: CCCCCCCCC(CCCCCCCC)OC(=O)CCCCCCCN(CCCCCCCC(=O)OCCCCCCCCC(C)C)CCOC(=O)CCCN(C)CCO[Si](C)(C)C(C)(C)C]

To a stirred solution of 4-({2-[(tert-butyl)bis(methyl)siloxy]ethyl}-N-methylamino)butyric acid (0.4 g, 4 eq., 1.45 mmol) in dichloromethane (10 mL), was added N,N-dimethyl-4-pyridylamine (177 mg, 4 eq., 1.45 mmol) and 1-Ethyl-3-(3-dimethylaminopropyl) carbodiimide (278 mg, 4 eq., 1.45 mmol) and stirred for 15 minutes. Then, 1-octylnonyl 8-{(2-hydroxyethyl)[7-(9-methyldecyloxycarbonyl)heptyl]amino}octanoate (268 mg, 363 μmol) was added. The reaction mixture was stirred at room temperature for 16 h. TLC showed formation of new spots and the starting material was consumed. The reaction mixture was quenched with brine solution (20 mL) and extracted with DCM (2×20 mL). The organic layer was dried over sodium sulfate, filtered and concentrated under vacuum pressure. The crude was purified by flash column chromatography (silica gel, 0-2% ethyl acetate in hexane) to obtain 1-octylnonyl 8-({2-[4-({2-[(tert-butyl)bis(methyl)siloxy]ethyl}-N-methylamino)butyroxy]ethyl}[7-(9-methyldecyloxycarbonyl)heptyl]amino)octanoate (230 mg, Yield=63.63%) as colorless liquid. ¹H NMR (400 MHz, CDCl₃-d3): δ 4.85 (quin, J=6.0 Hz, 1H), 4.10 (t, J=6.4 Hz, 2H), 4.05 (t, J=6.8 Hz, 2H), 3.72-3.65 (m, 3H), 2.66 (t, J=6.4 Hz, 2H), 2.50 (t, J=6.4 Hz, 2H), 2.45-2.37 (m, 7H), 2.35-2.25 (m, 10H), 1.82-1.75 (m, 2H), 1.65-1.56 (m, 4H), 1.55-1.45 (m, 5H), 1.44-1.38 (m, 5H), 1.32-1.19 (m, 44H), 1.20-1.12 (m, 2H), 0.91-0.81 (m, 21H), 0.67 (s, 6H).
Example 30. Synthesis of heptadecan-9-yl 8-((2-((4-((2-hydroxyethyl)(methyl)amino)butanoyl)oxy)ethyl)(8-((9-methyldecyl)oxy)-8-oxooctyl)amino)octanoate

This example illustrates synthesis of:

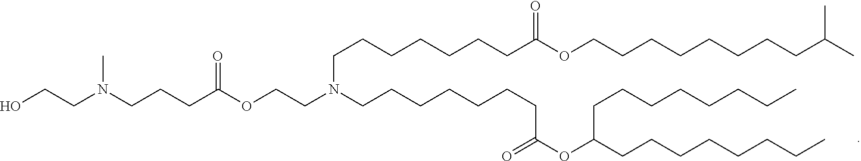 [SMILES: CCCCCCCCC(CCCCCCCC)OC(=O)CCCCCCCN(CCCCCCCC(=O)OCCCCCCCCC(C)C)CCOC(=O)CCCN(C)CCO]

To a stirred solution of 1-octylnonyl 8-({2-[4-({2-[(tert-butyl)bis(methyl)siloxy]ethyl}-N-methylamino)butyroxy]ethyl}[7-(9-methyldecyloxycarbonyl)heptyl]amino)octanoate (0.2 g, 201 μmol) in tetrahydrofuran (10 mL), was added pyridine hydrofluoride (1/1) (99.5 mg, 5 eq., 1 mmol) drop wise at 0° C. Reaction mixture was stirred at room temperature for 16 h. Progress of reaction was monitor by TLC and ELSD. The reaction mixture was quenched by saturated sodium bicarbonate to adjust pH 8, and extracted with ethyl acetate (3×25 mL). The organic layers were combine, dried over sodium sulphate, filtered and evaporated under reduce pressure. The crude material thus obtained was purify by flash column chromatography (silica gel, 0-5% gradient of MeOH in DCM) to get 1-octylnonyl 8-[(2-{4-[(2-hydroxyethyl)-N-methylamino]butyroxy}ethyl)[7-(9-methyldecyloxycarbonyl)heptyl]amino]octanoate (150 mg, Yield=70.3%) as pale yellow liquid. Compound was dissolved in dichloromethane (5 mL) under nitrogen atmosphere and filtered through hydrophobic PTFE syringe filter (25 mm×0.45 μm) using glass syringe to give desired compound. ¹H NMR (400 MHz, CDCl₃-d3): δ 4.85 (quin, J=6.4 Hz, 1H), 4.10 (t. J=6.4 Hz, 2H), 4.05 (t, J=6.8 Hz, 2H), 3.57 (t, J=5.2 Hz, 2H), 2.66 (t, J=6.0 Hz, 2H), 2.51 (t, J=5.2 Hz, 2H), 2.46-2.37 (m, 6H), 2.33 (t, J=7.2 Hz, 2H), 2.31-2.25 (m, 4H), 2.23 (s, 3H), 1.80 (quin, J=7.2 Hz, 1H), 1.67-1.56 (m, 6H), 1.55-1.45 (m, 4H), 1.45-1.37 (m, 4H), 1.36-1.21 (m, 48H), 1.18-1.08 (m, 2H), 0.91-0.82 (m, 12H). ELSD) analysis: Purity 98.31%, Calculated for C₅₃H₁₀₅N₂O₇, [M+H]=881.79, Observed=881.50 (m/z, M+H⁺).
Example 31. Synthesis of 7-{(2-hydroxyethyl)[5-(9-methyldecyloxycarbonyl)pentyl]amino}heptyl 2-octyldecanoate

This example illustrates synthesis of:

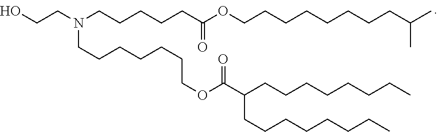 [SMILES: CCCCCCCCC(CCCCCCCC)C(=O)OCCCCCCCN(CCO)CCCCCC(=O)OCCCCCCCCC(C)C]

To a stirred solution of 9-methyldecyl 6-bromohexanoate (791 mg, 2.26 mmol, 1.0 eq.,) in acetonitrile (10.9 mL), N-ethylbis(isopropyl)amine (988 μL, 2.5 eq., 5.66 mmol) and 7-(2-hydroxyethylamino)heptyl 2-octyldecanoate (1 g, 2.26 mmol, 1.0 eq.,) was added and reaction mixture was stirred at 90° C. for 24 h. TLC showed starting materials were consumed and formed new spots. The reaction mass concentrated to get crude. The crude was purified by flash column chromatography (silica gel, 0-12% gradient of methanol in DCM) to get 7-{(2-hydroxyethyl)[5-(9-methyldecyloxycarbonyl)pentyl]amino}heptyl 2-octyldecanoate (150 mg, Yield=9.33%) as a colorless liquid. ¹H NMR (400 MHz, CDCl₃-d3): δ 4.07 (t, J=6.8 Hz, 4H), 3.92 (bs, 1H), 3.10-2.91 (bd, 4H), 2.34-2.25 (m, 3H), 1.85-1.72 (bs, 4H), 1.71-1.35 (m, 20H), 1.32-1.21 (m, 38H), 0.91-0.84 (m, 12H). ELSD analysis: Purity 96.99%, Calculated for C₄₄H₈₈NO₅, [M+H]=710.67, Observed=710.50 (m/z, M+H⁺).
Example 32. Synthesis of 7-((2-((4-(dimethylamino)butanoyl)oxy)ethyl)(6-((9-methyldecyl)oxy)-6-oxohexyl)amino)heptyl 2-octyldecanoate

This example illustrates synthesis of:

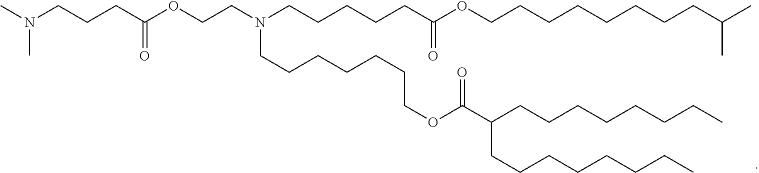 [SMILES: CCCCCCCCC(CCCCCCCC)C(=O)OCCCCCCCN(CCCCCC(=O)OCCCCCCCCC(C)C)CCOC(=O)CCCN(C)C]

To a stirred solution of 4-(dimethylamino)butyric acid (111 mg, 4 eq., 845 μmol) in dichloromethane (721 μL), {3-[cyano(ethyl)amino]propyl}dimethylazanium chloride (162 mg, 4 eq., 845 μmol) and 4-(dimethylamino)pyridin-1-ium (104 mg, 4 eq., 845 μmol) was added and stirred for 15 minutes. Then 7-{(2-hydroxyethyl)[5-(9-methyldecyloxycarbonyl)pentyl]amino}heptyl 2-octyldecanoate (150 mg, 211 μmol) was added. The reaction mixture was stirred at room temperature for 16 h. The reaction mixture was quenched with brine solution (10 mL) and extracted with DCM (2×15 mL). The combined organic layer was dried over sodium sulphate, filtered and concentrated under reduced pressure. The crude thus obtained was diluted with pentane and washed with acetonitrile (3×50 mL). Pentane layer was evaporated and distilled (temperature below 30° C.) to get 7-({2-[4-(dimethylamino)butyroxy]ethyl}[5-(9-methyldecyloxycarbonyl)pentyl]amino)heptyl 2-octyldecanoate (80 mg, Yield=46%) as a colorless liquid. ¹H NMR (400 MHz, CDCl₃-d3): δ 4.12 (t, J=6.4 Hz, 2H), 4.07-4.03 (m, 4H), 2.66 (t, J=6.4 Hz, 2H), 2.48-2.41 (m, 4H), 2.35 (t, J=7.2 Hz, 6H), 2.33-2.25 (m, 6H), 1.87-1.81 (m, 2H), 1.68-1.55 (m, 8H), 1.51-1.38 (m, 7H), 1.35-1.21 (m, 43H), 1.21-1.12 (m, 2H), 0.91-0.85 (m, 12H). ELSD analysis: Purity 99.10%, Calculated for C₅₀H₉₉N₂O₆, [M+H]=823.75, Observed=823.55 (m/z, M+H⁺).
Example 33. Synthesis of heptadecan-9-yl 8-((6-((9-methyldecyl)oxy)-6-oxohexyl)(2,2,3,3,7-pentamethyl-11-oxo-4,12-dioxa-7-aza-3-silatetradecan-14-yl)amino)octanoate

This example illustrates synthesis of:

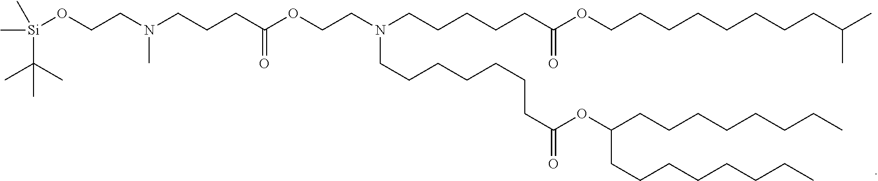 [SMILES: CCCCCCCCC(CCCCCCCC)OC(=O)CCCCCCCN(CCCCCC(=O)OCCCCCCCCC(C)C)CCOC(=O)CCCN(C)CCO[Si](C)(C)C(C)(C)C]

To a stirred solution of 4-({2-[(tert-butyl)bis(methyl)siloxy]ethyl}-N-methylamino)butyric acid (465 mg, 1.69 mmol, 4 eq) in dichloromethane (20 mL) was add N,N-dimethyl-4-pyridylamine (155 mg, 1.27 mmol, 3 eq) and 1-Ethyl-3-(3-dimethylaminopropyl) carbodiimide (486 mg, 2.53 mmol, 6 eq) and stirred for 15 min. Then 1-octylnonyl 8-{(2-hydroxyethyl)[5-(9-methyldecyloxycarbonyl)pentyl]amino}octanoate (0.3 g, 422 μmol, 1.0 eq) was added. The reaction mixture was stirred at RT for 16 h. After 16 h, TLC showed formation of new spot and the starting material was consumed. The reaction mixture was quenched with brine solution and extracted with DCM (20 mL×2). The organic layer was dried over sodium sulfate, filtered and concentrated under vacuum pressure to get the crude product. The crude mixture was purified under combi-flash chromatography by using 0-2% ethyl acetate in hexane. The desired fractions were collected and distilled to get 1-octylnonyl 8-({2-[4-({2-[(tert-butyl)bis(methyl)siloxy]ethyl}-N-methylamino)butyroxy]ethyl}[5-(9-methyldecyloxycarbonyl)pentyl]amino)octanoate (0.2 g, Yield=48.9%) as colorless liquid. ELSD analysis: Purity 98.17%, Calculated for C₅₇H₁₁₄N₂O₇Si, [M+H]=967.84, Observed=967.65 (m/z, M+H⁺).
Example 34. Synthesis of heptadecan-9-yl 8-((2-((4-((2-hydroxyethyl)(methyl)amino)butanoyl)oxy)ethyl)(6-((9-methyldecyl)oxy)-6-oxohexyl)amino)octanoate

This example illustrates synthesis of:

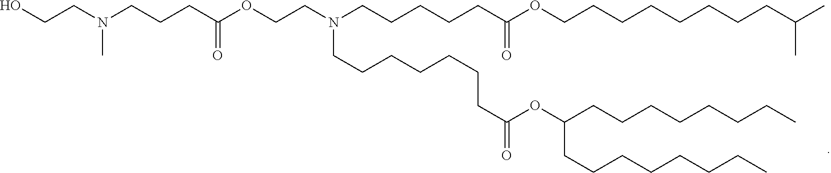 [SMILES: CCCCCCCCC(CCCCCCCC)OC(=O)CCCCCCCN(CCCCCC(=O)OCCCCCCCCC(C)C)CCOC(=O)CCCN(C)CCO]

To a stirred solution of 1-octylnonyl 8-({2-[4-({2-[(tert-butyl)bis(methyl)siloxy]ethyl}-N-methylamino)butyroxy]ethyl}[5-(9-methyldecyloxycarbonyl)pentyl]amino)octanoate (120 mg, 124 μmol) in tetrahydrofuran (10 mL), added hydrogen fluoride-pyridine (1/1) (55.9 μL, 5 eq., 620 μmol) drop wise at 0° C. Reaction mixture allowed to stir for 16 h at RT. Progress of reaction was monitor by TLC and ELSD. After completion of reaction, reaction mass quenched by saturated sodium bicarbonate up to pH 8, and extracted with ethyl acetate (3×25 mL). The organic layers were combine, dried over sodium sulphate, filtered and evaporated under reduce pressure to get crude, which was purify over silica (0-5% MeOH in DCM) to give 1-octylnonyl 8-[(2-{4-[(2-hydroxyethyl)-N-methylamino]butyroxy}ethyl)[5-(9-methyldecyloxycarbonyl)pentyl]amino]octanoate (105 mg. Yield=99%) as pale yellow liquid. Compound was dissolved in dichloromethane (5 mL) under nitrogen atmosphere and filtered through hydrophobic PTFE syringe filter (25 mm×0.45 μm) using glass syringe to give desired compound. ¹H NMR (400 MHz, CDCl₃): δ 4.85 (quin, J=6.4 Hz, 1H), 4.12 (t, J=6.4 Hz, 2H), 4.05 (t, J=6.8 Hz, 2H), 3.62-3.59 (m, 2H), 2.69 (t, J=6.0 Hz, 2H), 2.58 (t, J=6.0 Hz, 2H), 2.52-2.49 (m, 2H), 2.48-2.43 (m, 4H), 2.35 (t, J=7.2 Hz, 2H), 2.31-2.25 (m, 7H), 1.88-1.81 (m, 2H), 1.66-1.54 (m, 6H), 1.52-1.42 (m, 6H), 1.40 (s, 1H), 1.38 (b, 2H), 1.34-1.25 (m, 44H), 1.16-1.12 (m, 2H), 0.89-0.85 (m, 12H) ELSD analysis: Purity 97.96%, Calculated for C₅₁H₁₀₀N₂O₇, [M+H]=853.75, Observed=853.60 (m/z, M+H⁺).
Example 35. Synthesis of 1-octylnonyl 8-{(2-hydroxyethyl)[5-(9-methyldecyloxycarbonyl)pentyl]amino}octanoate

This example illustrates synthesis of:

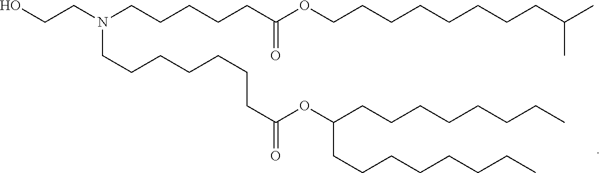 [SMILES: CCCCCCCCC(CCCCCCCC)OC(=O)CCCCCCCN(CCO)CCCCCC(=O)OCCCCCCCCC(C)C]

To a stirred solution of compound 9-methyldecyl 6-bromohexanoate (1 g, 2.86 mmol) in acetonitrile (15 mL), N-ethylbis(isopropyl)amine (771 μL, 1.5 eq., 4.29 mmol) and 1-octylnonyl 8-(2-hydroxyethylamino)octanoate (1.26 g, 2.86 mmol) were added. The reaction mixture was stirred at 95° C. for 16 h. After 16 h, TLC and ELSD showed formation of new spots and starting materials were consumed. The reaction mass was concentrated to get the crude mixture. The crude was purified by flash column chromatography (silica gel, 0-13% gradient of methanol in DCM) to get 1-octylnonyl 8-{(2-hydroxyethyl)[5-(9-methyldecyloxycarbonyl)pentyl]amino}octanoate (0.8 g, Yield=39.35%) as a colorless liquid. ¹H NMR (400 MHz, CDCl₃): δ 4.87 (quin, J=6.0 Hz, 1H), 4.07 (t, J=7.2 Hz, 2H), 3.60 (b, 2H), 2.66-2.54 (b, 5H), 2.33-2.27 (m, 4H), 1.69-1.61 (m, 6H), 1.59-1.51 (m, 8H), 1.49-1.43 (m, 44H), 1.32-1.27 (m, 2H), 0.91-0.78 (m, 12H). ELSD analysis: Purity 99.40%, Calculated for C₄₄H₈₈NO₅, [M+H]=710.66, Observed=710.55 (m/z. M+H⁺).
Example 36. Synthesis of heptadecan-9-yl 8-((2-((4-(dimethylamino)butanoyl)oxy)ethyl)(6-((9-methyldecyl)oxy)-6-oxohexyl)amino)octanoate

This example illustrates synthesis of:

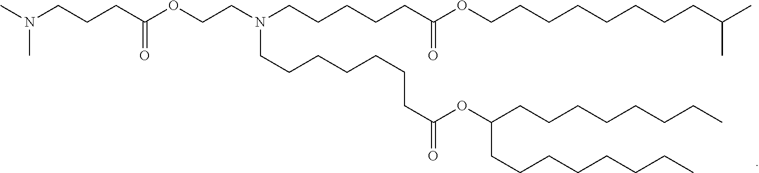 [SMILES: CCCCCCCCC(CCCCCCCC)OC(=O)CCCCCCCN(CCCCCC(=O)OCCCCCCCCC(C)C)CCOC(=O)CCCN(C)C]

To a stirred solution of 4-(dimethylamino)butyric acid-hydrogen chloride (1/1) (189 mg, 4 eq., 1.13 mmol) in dichloromethane (10 mL, 156 mmol), was added 1-Ethyl-3-(3-dimethylaminopropyl) carbodiimide (324 mg, 6 eq., 1.69 mmol) and N,N-dimethyl-4-pyridylamine (86 mg, 2.5 eq., 704 μmol) and stirred for 15 min, then 1-octylnonyl 8-{(2-hydroxyethyl)[5-(9-methyldecyloxycarbonyl)pentyl]amino}octanoate (0.2 g. 282 μmol) was added. The reaction mixture was stirred at r.t. for 48 h. The progress of reaction was monitored by ELSD/TLC (SM was consumed). The reaction mixture was quenched with brine solution and extracted with DCM (2×10 mL). The combined organic layer was dried over sodium sulphate, filtered, and concentrated to get the crude mixture. The crude was diluted with pentane and washed with acetonitrile (3×10 ml). Pentane layer was evaporated and distilled (temp. below 30° C.) to get 1-octylnonyl 8-({2-[4-(dimethylamino)butyroxy]ethyl}[5-(9-methyldecyloxycarbonyl)pentyl]amino)octanoate (140 mg, Yield=60.38%) as colorless liquid. Compound was dissolved in dichloromethane (5 mL) under nitrogen atmosphere and filtered through hydrophobic PTFE syringe filter (25 mm×0.45 μm) using glass syringe to give desired compound. ¹H NMR (400 MHz, CDCl₃): δ 4.85 (quin, J=6.4 Hz, 1H), 4.12 (t, J=6.4 Hz, 2H), 4.05 (t, J=6.8 Hz, 2H), 2.68 (t, J=6.0 Hz, 2H), 2.47-2.36 (m, 12H), 2.28 (t, J=7.6 Hz, 2H), 1.91-1.87 (m, 2H), 1.66-1.57 (m, 6H), 1.54-1.38 (m, 9H), 1.38-1.25 (m, 44H), 1.17-1.13 (m, 2H), 0.89-0.85 (m, 12H) ELSD analysis: Purity 99.16%, Calculated for C₅₀H₉₈N₂O₆, [M+H]=823.60, Observed=823.60 (m/z, M+H⁺).
Example 37. Synthesis of 9-hydroxynonyl acetate

This example illustrates synthesis of:

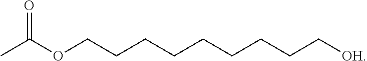

To a stirred solution of 1,9-nonanediol (150 g, 936 mmol) in tetrahydrofuran (1.24 L), pyridine (151 mL, 2 eq., 1.87 mol) and acetyl acetate (91.9 mL, 936 mmol) was added at 0° C. and stirred at 25° C. for 16 h. The reaction mixture was quenched with aqueous sodium bicarbonate solution (1.0 L) and extracted with ethyl acetate (5×500 mL). The organic layer was dried over sodium sulphate, filtered and concentrated to get crude. The crude was purified by flash column chromatography (silica gel, 0-50% gradient of ethyl acetate in hexane) to yield 9-hydroxynonyl acetate (80 g, Yield=42.25%) as a colorless liquid. ¹H NMR (400 MHz, CDCl₃-d3): δ 4.04 (t, J=6.8 Hz, 2H), 3.63 (t, J=6.8 Hz, 2H), 2.04 (s, 3H), 1.65-1.51 (m, 4H), 1.41-1.25 (m, 10H).
Example 38. Synthesis of 9-fluorononyl acetate

This example illustrates synthesis of:

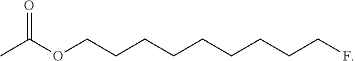

Compound 9-hydroxynonyl acetate (25 g, 124 mmol) in dichloromethane (250 mL) was cooled to −78° C. and N,N-diethyl(trifluorothio)amine (24.9 mL, 1.5 eq., 185 mmol) was added dropwise. The reaction mixture was allowed to warm to room temperature and stirring was continued for 16 h. Then, reaction mass was cooled to −78° C. and added dropwise aqueous sodium bicarbonate solution (35 mL) and extracted with ethyl acetate (3×100 mL). The organic layer was collected dried over sodium sulphate, filtered and evaporated to dryness. The crude was purified by flash column chromatography (silica gel, 0-30% gradient of ethyl acetate in hexane) to obtain 9-fluorononyl acetate (23 g. Yield=91.1%) as colorless liquid. ¹H NMR (400 MHz, CDCl₃-d3): δ 4.49 (t, J=6.0 Hz, 1H), 3.37 (t, J=6.0 Hz, 1H), 4.05 (t, J=6.8 Hz, 2H), 2.04 (s, 3H), 1.78-1.58 (m, 4H), 1.45-1.28 (m, 10H).
Example 39. Synthesis of 9-fluoro-1-nonanol

This example illustrates synthesis of:

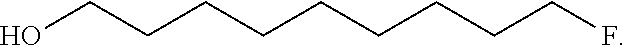 [SMILES: OCCCCCCCCCF]

Compound 9-fluorononyl acetate (20 g, 97.9 mmol) in tetrahydrofuran (124 mL) was cooled to −78° C. and aluminium (3+) lithium tetrahydride (7.43 g, 2 eq., 196 mmol) was added dropwise. After complete addition, reaction mixture was stirred at 25° C. for 16 h. TLC showed starting materials were consumed and formed new spots. The reaction mass was cooled to −10° C. and quenched with aqueous ammonium chloride (100 mL). Then, reaction mass was filtered through celite bed. The filtrate was collected and evaporated under reduced pressure. The crude was purified by flash column chromatography (silica gel, 0-12% gradient of ethyl acetate in hexane) to obtain 9-fluoro-1-nonanol (15 g, Yield=94.43%) as a colorless liquid. ¹H NMR (400 MHz, CDCl₃-d3): δ 4.49 (t, J=6.0 Hz, 1H), 3.37 (t, J=6.0 Hz, 1H), 3.63 (t, J=6.8 Hz, 2H), 1.75-1.61 (m, 2H), 1.58-1.51 (m, 2H), 1.45-1.28 (m, 10H).
Example 40. Synthesis of 9-fluorononanal

This example illustrates synthesis of:

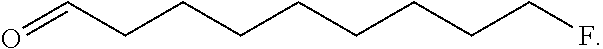 [SMILES: O=CCCCCCCCCF]

To a stirred solution of 9-fluoro-1-nonanol (15.6 g, 96.1 mmol) in dichloromethane (189 mL), was added 1-pyridylium chloridotrioxidochromate(1−) (41.5 g, 2 eq., 192 mmol) at 0° C. and reaction was continued at 25° C. for 2 h. TLC showed starting materials were consumed and formed new spots. The reaction mixture was diluted with pentane and stirred for 30 minutes. Reaction mixture was filtered through celite bed and wash with pentane (3×100 mL). The organic layer collected and evaporated under reduced pressure. The crude was purified by flash column chromatography (silica gel, 20-25% gradient of ethyl acetate in hexane) to obtain 9-fluorononanal (5.5 g, Yield=35.7%) as colorless liquid. ¹H NMR (400 MHz, CDCl₃-d3): δ 9.76 (s, 1H), 4.49 (t, J=6.0 Hz, 1H), 4.37 (t, J=6.0 Hz, 1H), 2.42 (td, J₁=7.2 Hz, J₂=1.6 Hz, 1H), 2.35 (t, J=7.2 Hz, 1H), 1.74-1.63 (m, 4H), 1.45-1.28 (m, 8H).
Example 41. Synthesis of 1-fluoro-9-heptadecanol

This example illustrates synthesis of:

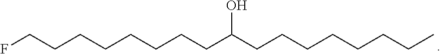 [SMILES: CCCCCCCCC(O)CCCCCCCCF]

9-fluorononanal (5.5 g, 34.3 mmol) in tetrahydrofuran (20 mL) was cooled down to −78° C. Then Octyl magnesium bromide (14.9 g, 2 eq., 68.7 mmol) was slowly added at −78° C. and reaction mixture was allowed to warm to 25° C. and was stirred for 1 h. Then reaction mixture was stirred at 55° C. for 2 h. TLC showed formation of new spots. Reaction mixture was quenched with water and filtered through celite bed. The organic layer was collected, dried over sodium sulphate, filtered and evaporated to dryness. The crude was purified by flash column chromatography (silica gel, 0-15% gradient of ethyl acetate in hexane) to obtain 1-fluoro-9-heptadecanol (4.5 g, Yield=47.77%) as a yellow liquid. ¹H NMR (400 MHz, CDCl₃-d3): δ 4.49 (t, J=6.0 Hz, 1H), 4.37 (t, J=6.0 Hz, 1H), 3.60-3.52 (m, 1H), 1.78-1.61 (m, 2H), 1.51-1.25 (m, 27H), 0.87 (t, J=6.8 Hz, 3H).
Example 42. Synthesis of 9-fluoro-1-octylnonyl 8-bromooctanoate

This example illustrates synthesis of:

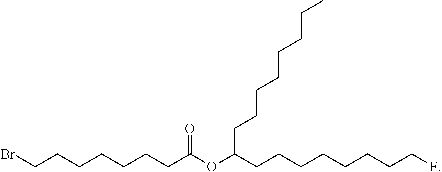 [SMILES: CCCCCCCCC(CCCCCCCCF)OC(=O)CCCCCCCBr]

To a stirred solution of 8-bromooctanoic acid (7.32 g, 2 eq., 32.8 mmol) in dichloromethane (146 mL), {3-[cyano(ethyl)amino]propyl}dimethylazanium chloride (12.6 g, 4 eq., 65.6 mmol) and 4-(dimethylamino)pyridin-1-ium (6.06 g, 3 eq., 49.2 mmol) was added and stirred for 15 minutes. Then, 1-fluoro-9-heptadecanol (4.5 g, 1 eq., 16.4 mmol) was added and reaction mixture was stirred at 25° C. for 16 h. TLC showed formation of new spots and starting material was consumed. Reaction mixture was quenched with brine solution (50 mL) and extracted with DCM (3×50 mL). The organic layer was collected, dried over sodium sulphate, filtered and evaporated to dryness. The crude was purified by flash column chromatography (silica gel, 0-10% gradient of ethyl acetate in hexane) to obtain 9-fluoro-1-octylnonyl 8-bromooctanoate (3 g, Yield=38.16%) as a colorless liquid. ¹H NMR (400 MHz, CDCl₃-d3): δ 4.86 (quin, J=6.0 Hz, 1H), 4.49 (t, J=6.0 Hz, 1H), 4.37 (t, J=6.4 Hz, 1H), 3.52 (t, J=6.8 Hz, 1H), 3.39 (t, J=6.8 Hz, 1H), 2.28 (t, J=7.6 Hz, 2H), 1.93-1.71 (m, 3H), 1.65-1.61 (m, 3H), 1.52-1.48 (m, 4H), 1.41-1.35 (m, 10H), 1.32-1.17 (m, 18H), 0.87 (t, J=6.8 Hz, 3H).
Example 43. Synthesis of 1-fluoroheptadecan-9-yl 8-((2-hydroxyethyl)(8-((9-methyldecyl)oxy)-8-oxooctyl)amino)octanoate

This example illustrates synthesis of:

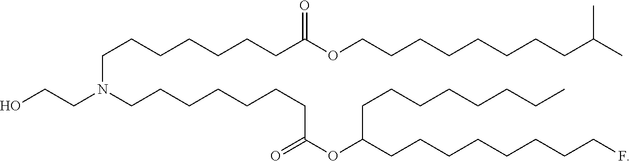 [SMILES: CCCCCCCCC(CCCCCCCCF)OC(=O)CCCCCCCN(CCO)CCCCCCCC(=O)OCCCCCCCCC(C)C]

To a stirred solution of 9-methyldecyl 8-(2-hydroxyethylamino)octanoate (447 mg, 1.25 mmol) in acetonitrile (4.47 mL), N-ethylbis(isopropyl)amine (668 μL, 3 eq., 3.75 mmol) and 9-fluoro-1-octylnonyl 8-bromooctanoate (0.6 g, 1.25 mmol) was added and reaction mixture was stirred at 90° C. for 16 h. ELSD showed 39% of desired product mass. Reaction mixture was concentrated under reduced pressure. The crude was purified by flash column chromatography (silica gel, 0-10% gradient of methanol in DCM) to obtain desired compounds (0.25 g) as a salt. Then we performed desalting process. Compound obtained (0.25 g) after flash column chromatography was dissolved in dichloromethane (10 mL) and added anhydrous potassium carbonate (5 eq.) under nitrogen atmosphere and stirred vigorously for 2 h. Then, reaction mixture was filtered through hydrophobic PTFE syringe filter (25 mm×0.45 μm) using glass syringe to get 9-fluoro-1-octylnonyl 8-{(2-hydroxyethyl)[7-(9-methyldecyloxycarbonyl)heptyl]amino}octanoate (0.2 g, Yield=21.14%) as a colorless liquid. ¹H NMR (400 MHz, CDCl₃-d3): δ 4.86 (quin, J=6.4 Hz, 1H), 4.48 (t, J=6.4 Hz, 1H), 4.37 (t, J=6.0 Hz, 1H), 4.05 (t, J=6.8 Hz, 2H), 3.51 (t, J=5.6 Hz, 2H), 2.55 (t, J=5.6 Hz, 2H), 2.42 (t, J=7.2 Hz, 4H), 2.30-2.24 (m, 4H), 1.75-1.6 (m, 7H), 1.52-1.38 (m, 10H), 1.35-1.22 (m, 45H), 1.18-1.12 (m, 2H), 0.89-0.84 (m, 9H). ELSD analysis: Purity 99.94%, Calculated for C₄₆H₉₁FNO₅, [M+H]=756.69, Observed=756.80 (m/z, M+H⁺).
Example 44. Synthesis of 9-(benzyloxy)-1-nonanol

This example illustrates synthesis of:

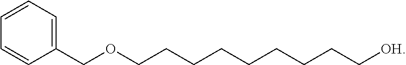 [SMILES: OCCCCCCCCCOCc1ccccc1]

To a stirred solution of 1,9-nonanediol (10 g, 62.4 mmol, 1.0 eq) in dimethylformamide (143 mL) was added sodium hydride (2.25 g, 93.6 mmol, 1.5 eq) at 0° C. and stirred for 30 min at same temperature. Then (bromomethyl)benzene (7.62 mL, 62.4 mmol, 1.0 eq) was added and the reaction mixture was stirred at r.t. for 16 h. After completion of the reaction (monitored by TLC), the reaction mixture was quenched with ice-cooled water (200 ml) and extracted with ethylacetate (4×100 mL). The resulting organic layer was dried over Na₂SO₄, and concentrated under reduce pressure, and the crude was purified by flash column chromatography (SiO₂: 0-15% Ethyl acetate in Hexane), to give 9-(benzyloxy)-1-nonanol (9.5 g, Yield=60.8%) as a colorless liquid. ¹H NMR (400 MHz, DMSO-d6): δ 7.36-7.25 (m, 5H), 4.43 (s, 2H), 4.32 (t, J=4 Hz, 1H), 3.42-3.36 (m, 4H), 1.54-1.49 (m, 2H), 1.37 (t, J=6 Hz, 2H), 1.24 (bs, 10H).
Example 45. Synthesis of 9-(benzyloxy)nonanal

This example illustrates synthesis of:

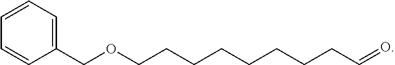 [SMILES: O=CCCCCCCCCOCc1ccccc1]

To a stirred solution of 9-(benzyloxy)-1-nonanol (10 g, 39.9 mmol, 1.0 eq) in dichloromethane (250 mL) was add 1-pyridylium chloridotrioxidochromate (17.2 g, 79.9 mmol, 2 eq) at 0° C. and the reaction mixture was stirred at r.t. for 2 h. After completion of the reaction (monitored by TLC). The reaction mixture was diluted with pentane (500 ml) and stirred for 30 min. After 30 min, filtered through Celite bed and wash with pentane (3×250 ml). The organic layer collected and concentrated under reduced pressure. The crude was purified by flash column chromatography (SiO₂: 0-25% ethyl acetate in hexane), to give 9-(benzyloxy)nonanal (7 g. Yield=70.57%) as a colorless liquid. ¹H NMR (400 MHz, CDCl3-d3): δ 9.76 (t, J=1.6 Hz, 1H), 7.37-7.26 (m, 5H), 4.51 (s, 2H), 3.46 (t, J=8.0 Hz, 2H), 2.44-2.40 (m, 2H), 1.64-1.57 (m, 4H), 1.36-1.32 (m, 8H).

### Example 46. Synthesis of 11-(benzyloxy)-2-methyl-2-undecene

This example illustrates synthesis of:

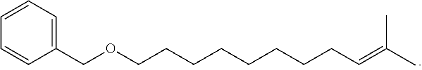 [SMILES: CC(C)=CCCCCCCCCOCc1ccccc1]

To a stirred solution of isopropyltriphenylphosphonium bromide (15.5 g, 40.1 mmol, 1.2 eq) in tetrahydrofuran (80 mL) was added lithium 1-butanide (3.21 g, 50.1 mmol, 1.5 eq) dropwise at −78° C. and the reaction mixture was stirred at room temperature for 1 hr, then the mixture was cooled again to −78° C. and 9-(benzyloxy)nonanal (8.3 g, 33.4 mmol, 1.0 eq) (dissolved in THF) was added dropwise. The reaction mixture was stirred at room temperature for 16 h. After completion of reaction (monitored by TLC), the reaction mixture was quenched with aq. ammonium chloride solution (200 mL) and extracted with ethyl acetate (3×100 mL) times. The organic layer was collected and dried over sodium sulphate, filtered and concentrated under vacuum to get the crude mixture. The crude was purified through combi-flash chromatography (SiO₂: 0-15% Ethyl acetate in Hexane), to get 11-(benzyloxy)-2-methyl-2-undecene (6.3 g, Yield=68.69%) as a colorless liquid. ¹H NMR (400 MHz, CDCl₃-d3): δ 7.32-7.25 (m, 5H), 5.09 (t, J=1.2 Hz, 1H), 4.48 (s, 2H), 3.44 (t, J=6.8 Hz, 2H), 1.94-1.92 (m, 2H), 1.66 (s, 3H), 1.61-1.55 (s, 4H), 1.35-1.26 (m, 9H).
Example 47. Synthesis of 10-methyl-1-undecanol

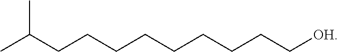

This example illustrates synthesis of: To a stirred solution of 11-(benzyloxy)-2-methyl-2-undecene (6 g, 21.9 mmol, 1.0 eq) in tetrahydrofuran (30 mL) and methanol (30 mL) was degassed with nitrogen for 5-10 min, then Pd/C (5.82 g, 54.7 mmol, 2.5 eq) was added and stirred at r.t. for 16 h under Hydrogen atmosphere. TLC showed consumption of starting material and formation of a new spot. The reaction mixture was filtered through celite bed and washed with mixture of MeOH:THF (3×200 ml). The filtrate was collected and concentrate to get the crude product which was purified through combi-flash chromatography (SiO₂: 0-15% Ethyl acetate in Hexane), to give 10-methyl-1-undecanol (2 g, Yield=49.09%) as colorless liquid. ¹H NMR (400 MHz, CDCl₃-d3): δ 3.64 (t, J=6.8 Hz, 2H), 1.60-1.47 (m, 3H), 1.33-1.25 (m, 13H), 1.15-1.20 (m, 2H), 0.86-0.85 (d, J=6.8 Hz, 6H).

Example 48. Synthesis of 10-methylundecanoic acid

This example illustrates synthesis of:

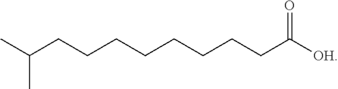

To a stirred solution of chromamethanetrione-sulfuric acid (10 mL, 65.6 mmol) and water (10 mL) was cooled to −10° C., 10-methyl-1-undecanol (1 g, 5.37 mmol, 1 eq.) dissolved in acetone (10 mL) added dropwise to the solution. The reaction mixture was stirred for overnight at room temperature. After completion of the reaction (monitored by TLC). Reaction mixture was dilute with water (100 ml) and extracted with diethylether (5×100 ml). The resulting organic layer was dried over Na₂SO₄, and concentrated under reduce pressure, and the crude was purified by flash column chromatography (SiO2: 0-25% Ethyl acetate in Hexanes), to get 10-methylundecanoic acid (500 mg. Yield=46.51%) as a white crystal. ¹H NMR (400 MHz, CDCL3-d3): δ 9.97 (s, 1H), 2.35 (t, J=7.6 Hz, 2H), 1.67-1.59 (m, 2H), 1.54-1.47 (m 1H), 1.32-1.25 (m, 10H), 1.15-1.11 (m, 2H), 0.86 (d, J=6.8 Hz, 6H).
Example 49. Synthesis of 7-bromoheptyl 10-methylundecanoate

This example illustrates synthesis of:

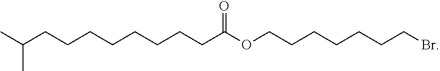 [SMILES: CC(C)CCCCCCCCC(=O)OCCCCCCCBr]

To a stirred solution of 10-methylundecanoic acid (0.5 g, 2.5 mmol, 1.0 eq) in dichloromethane (8.52 mL), was added 4-(dimethylamino)pyridin-1-ium (615 mg, 4.99 mmol, 2 eq.) and {3-[cyano(ethyl)amino]propyl}dimethylazanium chloride (1.91 g, 9.98 mmol, 4 eq.) to it. The reaction was stirred for 15 min then 7-bromo-1-heptanol (487 mg, 2.5 mmol, 1.0 eq) was added. The reaction mixture was again stirred at r.t. for 48 h. The progress of reaction was monitored by TLC (SM was consumed). Water (150 mL) was added to the reaction mixture, and extracted with DCM (3×250 mL). The resulting organic layer was dried over Na₂SO₄, and concentrated under reduce pressure, and the crude was purified by flash column chromatography (SiO₂: 0-2% Ethyl acetate in Hexanes), to give 7-bromoheptyl 10-methylundecanoate (0.45 g, 1.06 mmol) as a colorless liquid. ¹H NMR (400 MHz, CDCl₃-d3): δ 4.05 (t, J=6.4 Hz, 2H), 3.55-3.39 (m, 2H), 2.29 (t, J=7.6 Hz, 2H), 1.89-1.82 (m, 2H), 1.64-1.54 (m, 4H), 1.49-1.42 (m, 3H), 1.36-1.28 (m, 12H), 1.25-1.15 (m, 2H), 0.86 (d, J=6.8 Hz, 6H).
Example 50. Synthesis of 7-((8-(heptadecan-9-yloxy)-8-oxooctyl) (2-hydroxyethyl)amino)heptyl 10-methylundecanoate

This example illustrates synthesis of:

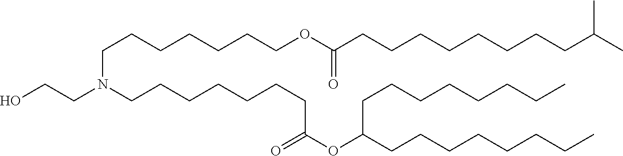 [SMILES: CCCCCCCCC(CCCCCCCC)OC(=O)CCCCCCCN(CCO)CCCCCCCOC(=O)CCCCCCCCC(C)C]

To a stirred solution of 1-octylnonyl 8-(2-hydroxyethylamino)octanoate (0.5 g, 1.13 mmol, 1.0 eq) in acetonitrile (5 mL), was added N-ethylbis(isopropyl)amine (395 μL, 2.26 mmol, 2.0 eq) and 7-bromoheptyl 10-methylundecanoate (427 mg, 1.13 mmol, 1.0 eq) to it. The reaction was stirred at 90° C. for 16 h. The progress of reaction was monitored by ELSD/TLC (63% product form in reaction mixture by ELSD). The reaction mixture was concentrated under reduced pressure and the crude was purified by flash column chromatography (SiO2: 0-8% Methanol in Dichloromethane), to give 7-{(2-hydroxyethyl) [7-(1-octylnonyloxycarbonyl)heptyl]amino}heptyl 10-methylundecanoate (400 mg, Yield=47.9%) as colorless liquid. 1H-NMR (400 MHz, CDCl₃-d3): δ 4.87-4.84 (m, 1H), 4.05 (t, J=6.8 Hz, 2H), 3.67 (bs, 2H), 2.74-2.62 (br, 6H), 2.30-2.25 (m, 4H), 1.62-1.47 (m, 15H), 1.32-1.25 (m, 46H), 1.16-1.13 (m, 2H), 0.89-0.84 (m, 12H). ELSD analysis: Purity 99.36%, Calculated C₄₆H₉₂NO₅, [M+H]=738.70, Observed=738.65 (m/z, M+H⁺).
Example 51. Synthesis of 7-((2-((4-(dimethylamino)butanoyl)oxy)ethyl)(8-(heptadecan-9-yloxy)-8-oxooctyl)amino)heptyl 10-methylundecanoate

This example illustrates synthesis of:

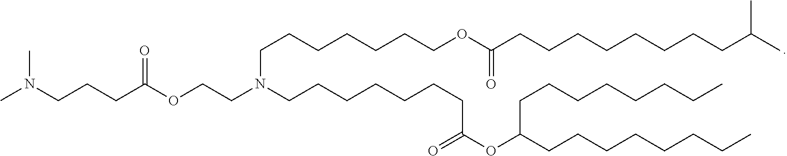 [SMILES: CCCCCCCCC(CCCCCCCC)OC(=O)CCCCCCCN(CCCCCCCOC(=O)CCCCCCCCC(C)C)CCOC(=O)CCCN(C)C]

To a stirred solution of 4-(dimethylamino)butyric acid (43.6 mg, 332 μmol, 2 eq) in dichloromethane (10 ml) was added {3-[cyano(ethyl)amino]propyl}dimethylazanium chloride (127 mg, 665 μmol, 4 eq) and 4-(dimethylamino)pyridin-1-ium (40.9 mg, 332 μmol, 2 eq) and stirred for 15 min, then 7-{(2-hydroxyethyl)[7-(1-octylnonyloxycarbonyl)heptyl]amino}heptyl 10-methylundecanoate (118 mg, 166 μmol, 1.0 eq) was added. The reaction mixture was stirred at r.t. for 16 h. The progress of reaction was monitored by ELSD/TLC (58% product form in reaction mixture with 41% DMAP and other Acetonitrile soluble impurity by ELSD). The reaction mixture was quenched with brine solution and extracted with DCM (2×100 mL). The combined organic layer was dried over sodium sulphate, filtered and concentrated to get the crude mixture. The crude was diluted with pentane and washed with acetonitrile (3×100 ml). Pentane layer was evaporated and distilled (temp. below 30° C.) to get 7-((2-((4-(dimethylamino)butanoyl)oxy)ethyl)(8-(heptadecan-9-yloxy)-8-oxooctyl)amino)heptyl 10-methylundecanoate (100 mg, Yield=73.1%) as pale brown liquid. ¹H-NMR (400 MHz, CDCl₃)-δ 4.88-4.82 (m, 1H), 4.14 (t, J=6.4 Hz, 2H), 4.04 (t, J=6.4 Hz, 2H), 2.71 (t, J=6.0 Hz, 2H), 2.57 (bs, 1H), 2.49-2.38 (m, 10H), 2.30-2.25 (m, 4H), 2.09 (br, 2H), 1.95-1.91 (m, 2H), 1.61 (t, J=6.0 Hz, 6H), 1.54-1.44 (m, 8H), 1.31-1.25 (m, 48H), 1.14-1.13 (m, 2H), 0.89-0.84 (m, 12H). ELSD analysis: Purity 97.18%, Calculated C₅₂H₁₀₃N₂O₆, [M+H]=851.78, Observed=851.70 (m/z, M+H⁺).
Example 52. Synthesis of 5-bromopentyl 10-methylundecanoate

This example illustrates synthesis of:

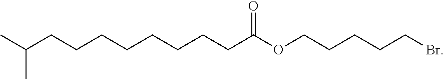 [SMILES: CC(C)CCCCCCCCC(=O)OCCCCCBr]

To a stirred solution of 10-methylundecanoic acid 12 (0.5 g, 2.5 mmol, 1.0 eq) in dichloromethane (10 mL) was added 4-(dimethylamino)pyridin-1-ium (307 mg. 2.5 mmol, 1.0 eq) and {3-[cyano(ethyl)amino]propyl}dimethylazanium chloride (957 mg, 4.99 mmol, 2 eq). The reaction stirred for 15 min then 5-bromo-1-pentanol 13 (417 mg, 2.5 mmol, 1.0 eq) was added. The reaction mixture was stirred at r.t. for 48 h. The progress of reaction was monitored by TLC (SM was consumed). Water (150 mL) was added to the reaction mixture and extracted with DCM (3×250 mL). The resulting organic layer was dried over Na₂SO₄, and concentrated under reduce pressure, and the crude was purified by flash column chromatography (SiO2: 0-2% Ethyl acetate in Hexanes) to give 5-bromopentyl 10-methylundecanoate 14 (450 mg, 1.29 mmol) as a colorless liquid. ¹H NMR (400 MHz, CDCl₃-d3): δ 4.07 (t, J=6.8 Hz, 2H), 3.56-3.39 (m, 2H), 2.31-2.27 (m, 2H), 1.93-1.69 (m, 2H), 1.67-1.54 (m, 3H), 1.51-1.46 (m, 2H), 1.28-1.25 (m, 12H), 1.36-1.25 (m, 2H), 0.86 (m, 6H).
Example 53. Synthesis of 5-((2-hydroxyethyl)(7-((2-octyldecanoyl)oxy)heptyl)amino)pentyl 10-methylundecanoate

This example illustrates synthesis of:

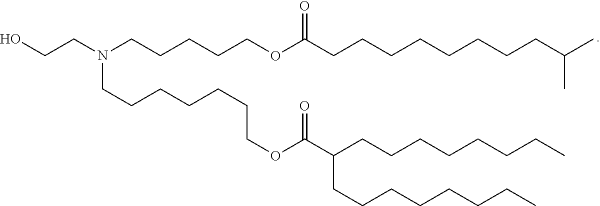 [SMILES: CCCCCCCCC(CCCCCCCC)C(=O)OCCCCCCCN(CCO)CCCCCOC(=O)CCCCCCCCC(C)C]

To a stirred solution of 7-(2-hydroxyethylamino)heptyl 2-octyldecanoate 16 (0.6 g, 1.36 mmol, 0.95 eq) in acetonitrile (8 mL), was added N-ethylbis(isopropyl)amine (625 μL, 3.58 mmol, 2.5 eq) and 5-bromopentyl 10-methylundecanoate 14 (0.5 g, 1.43 mmol, 1.0 eq). The reaction was stirred at 95° C. for 16 h. The progress of reaction was monitored by ELSD/TLC (70% product form in reaction mixture by ELSD). The reaction mixture was concentrated under reduced pressure and the crude was purified by flash column chromatography (SiO2: 0-8% Methanol in Dichloromethane), to get 5-((2-hydroxyethyl)(7-((2-octyldecanoyl)oxy)heptyl)amino)pentyl 10-methylundecanoate (430 mg, Yield=42.31%) as a colorless liquid. ELSD analysis: Purity 99.05%, Calculated C₄₄H₈₈NO₅, [M+H]=710.67, Observed=710.40 (m/z, M+H⁺).
Example 54. Synthesis of 5-((2-((4-(dimethylamino)butanoyl)oxy)ethyl)(7-((2-octyldecanoyl)oxy)heptyl)amino)pentyl 10-methylundecanoate

This example illustrates synthesis of:

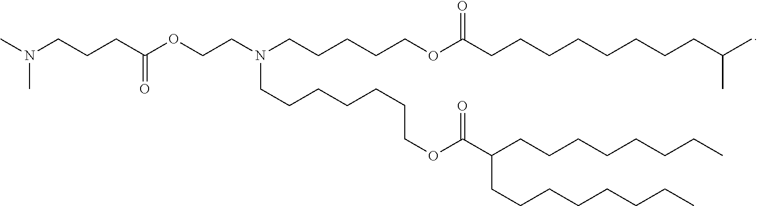 [SMILES: CCCCCCCCC(CCCCCCCC)C(=O)OCCCCCCCN(CCCCCOC(=O)CCCCCCCCC(C)C)CCOC(=O)CCCN(C)C]

To a stirred solution of 4-(dimethylamino)butyric acid 18 (318 mg, 2.42 mmol, 4 eq) in dichloromethane (10 mL), was added {3-[cyano(ethyl)amino]propyl}dimethylazanium chloride (464 mg, 2.42 mmol, 4 eq) and N,N-dimethyl-4-pyridylamine (148 mg, 2 eq., 1.21 mmol) and stirred for 15 min, then 5-{(2-hydroxyethyl)[7-(1-octylnonyl carbonyloxy)heptyl]amino}pentyl 10-methylundecanoate (430 mg, 605 μmol, 1.0 eq) was added. The reaction mixture was stirred at r.t. for 16 h. The progress of reaction was monitored by ELSD/TLC (46% product form in reaction mixture with 53% DMAP and other Acetonitrile soluble impurity by ELSD). The reaction mixture was quenched with brine solution and extracted with DCM (2×10 mL). The combined organic layer was dried over sodium sulphate, filtered and concentrated to get the crude mixture. The crude was diluted with pentane and washed with acetonitrile (3×10 ml). Pentane layer was evaporated and distilled (temp. below 30° C.) to get 5-({2-[4-(dimethylamino)butyroxy]ethyl}[7-(1-octylnonylcarbonyloxy)heptyl]amino)pentyl 10-methylundecanoate (248 mg, Yield=49.75%) as a pale brown liquid. ¹H-NMR (400 MHz, CDCl₃-d₃): δ 4.11 (t, J=6.4 Hz, 2H), 4.07-4.03 (m, 4H), 2.67 (t, J=6.4 Hz, 2H), 2.47-2.41 (m, 4H), 2.36-2.33 (m, 4H), 2.31-2.26 (m, 8H), 1.88-1.78 (m, 2H), 1.67-1.57 (m, 8H), 1.56-1.47 (m, 1H), 1.43-1.34 (m, 6H), 1.24 (br, 43H), 1.16-1.11 (m, 2H), 0.88-0.82 (m, 12H). ELSD analysis: Purity 99.36%, Calculated C₅₀H₉₉N₂O₆, [M+H]=823.74, Observed=823.70 (m/z, M+H⁺).
Example 55. Synthesis of 7-((2-hydroxyethyl)(7-((2-octyldecanoyl)oxy)heptyl)amino)heptyl 10-methylundecanoate

This example illustrates synthesis of:

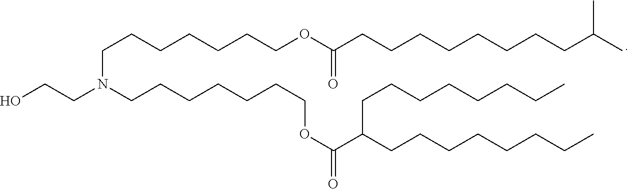 [SMILES: CCCCCCCCC(CCCCCCCC)C(=O)OCCCCCCCN(CCO)CCCCCCCOC(=O)CCCCCCCCC(C)C]

To a stirred solution of 7-bromoheptyl 10-methylundecanoate (450 mg, 1.19 mmol) in acetonitrile (5 mL, 95.7 mmol), was added N-ethylbis(isopropyl)amine (521 μL, 2.5 eq., 2.98 mmol) and 7-(2-hydroxyethylamino)heptyl 2-octyldecanoate (527 mg, 1.19 mmol). The reaction was stirred at 95° C. for 16 h. The progress of reaction was monitored by ELSD/TLC (30% product formation in reaction mixture by ELSD). The reaction mixture was concentrated under reduced pressure and the crude was purified by flash column chromatography (SiO2: 0-8% Methanol in Dichloromethane), to give 7-{(2-hydroxyethyl)[7-(1-octylnonyl carbonyloxy)heptyl]amino}heptyl 10-methylundecanoate (230 mg, Yield=26.13%) as colorless liquid. ¹H-NMR (400 MHz, CDCl₃-d3): δ 4.07 (s, 4H), 3.90 (bs, 2H), 3.03-2.93 (m, 6H), 2.33-2.26 (m, 3H), 1.62-1.47 (m, 11H), 1.45-1.40 (m, 3H), 1.36 (bs, 11H), 1.29-1.24 (m, 37H), 1.16-1.15 (m, 2H), 0.89-0.84 (m, 12H). ELSD analysis: Purity 99.41%, Calculated C₄₆H₉₂NO₅, [M+H]=738.70, Observed=738.65 (m/z, M+H⁺).
Example 56. Synthesis of 5-bromopentyl 10-methylundecanoate

This example illustrates synthesis of:

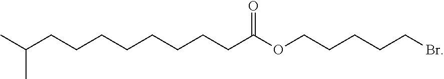 [SMILES: CC(C)CCCCCCCCC(=O)OCCCCCBr]

To a stirred solution of 10-methylundecanoic acid (0.5 g, 2.5 mmol, 1.0 eq) in dichloromethane (10 mL), was added 4-(dimethylamino)pyridin-1-ium (307 mg, 2.5 mmol, 1.0 eq) and {3-[cyano(ethyl)amino]propyl}dimethylazanium chloride (957 mg, 2 eq., 4.99 mmol). The reaction mixture was stirred for 15 min and then added 5-bromo-1-pentanol (417 mg, 2.5 mmol) to it. The reaction mixture was stirred at r.t. for 48 h. The progress of reaction was monitored by TLC (SM was consumed). Water (150 mL) was added to the reaction mixture and extracted with DCM (3×250 mL). The resulting organic layer was dried over Na₂SO₄, and concentrated under reduce pressure, and the crude was purified by flash column chromatography (SiO₂: 0-2% Ethyl acetate in Hexanes), to give 5-bromopentyl 10-methylundecanoate (450 mg, 1.29 mmol) as a colorless liquid. ¹H NMR (400 MHz, CDCl₃-d3): δ 4.07 (t, J=6.8 Hz, 2H), 3.56-3.39 (m, 2H), 2.31-2.27 (m, 2H), 1.93-1.69 (m, 2H), 1.67-1.54 (m, 3H), 1.51-1.46 (m, 2H), 1.28-1.25 (m, 12H), 1.36-1.25 (m, 2H), 0.86 (m, 6H).
Example 57. Synthesis of 5-((8-(heptadecan-9-yloxy)-8-oxooctyl)(2-hydroxyethyl)amino)pentyl 10-methylundecanoate

This example illustrates synthesis of:

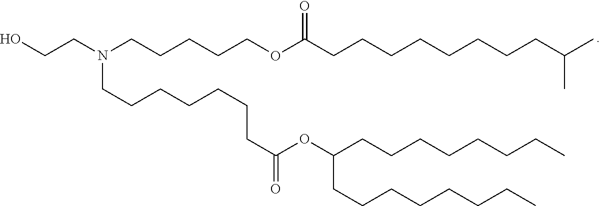 [SMILES: CCCCCCCCC(CCCCCCCC)OC(=O)CCCCCCCN(CCO)CCCCCOC(=O)CCCCCCCCC(C)C]

To a stirred solution of 1-octylnonyl 8-(2-hydroxyethylamino)octanoate (460 mg, 1.04 mmol) in acetonitrile (5 mL), was added N-ethylbis(isopropyl)amine (455 μL, 2.5 eq., 2.6 mmol) and 5-bromopentyl 10-methylundecanoate (364 mg, 1.04 mmol) to it. The reaction was stirred at 90° C. for 16 h. The progress of reaction was monitored by ELSD/TLC (83% product form in reaction mixture by ELSD). The reaction mixture was concentrated under reduced pressure and the crude was purified by flash column chromatography (SiO2: 0-8% Methanol in Dichloromethane), to get 5-{(2-hydroxyethyl) [7-(1-octylnonyloxy carbonyl)heptyl]amino}pentyl 10-methylundecanoate (270 mg, Yield=36.51%) as a colorless liquid. ¹H-NMR (400 MHz, CDCl₃-d3): δ 4.88-4.83 (m, 1H), 4.07 (t, J=6.4 Hz, 2H), 3.94 (bs, 2H), 3.00 (br, 6H), 2.31-2.26 (m, 4H), 1.81 (br, 3H), 1.70-1.49 (m, 15H), 1.45-1.25 (m, 40H), 1.15 (bs, 2H), 0.89-0.84 (m, 12H). ELSD analysis: Purity 99.84%, Calculated C₄₄H₈₈NO₅, [M+H]=710.67, Observed=710.65 (m/z, M+H⁺).
Example 58. Synthesis of 5-({2-[4-(dimethylamino)butyroxy]ethyl}[7-(1-octylnonyloxycarbonyl)heptyl]amino)pentyl 10-methylundecanoate

This example illustrates synthesis of:

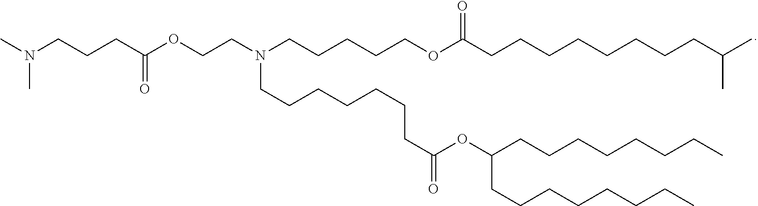 [SMILES: CCCCCCCCC(CCCCCCCC)OC(=O)CCCCCCCN(CCCCCOC(=O)CCCCCCCCC(C)C)CCOC(=O)CCCN(C)C]

To a stirred solution of 4-(dimethylamino)butyric acid (111 mg. 2 eq., 845 μmol) in dichloromethane (10 mL), was added {3-[cyano(ethyl)amino]propyl}dimethylazanium chloride (324 mg, 4 eq., 1.69 mmol) and 4-(dimethylamino)pyridin-1-ium (104 mg. 2 eq., 845 μmol) and stirred for 15 min, then added 5-{(2-hydroxyethyl)[7-(1-octylnonyloxycarbonyl)heptyl]amino}pentyl 10-methylundecanoate (0.3 g, 422 μmol). The reaction mixture was stirred at r.t. for 16 h. The progress of reaction was monitored by ELSD/TLC (48% product formation in reaction mixture by ELSD). The reaction mixture was quenched with brine solution and extracted with DCM (2×10 mL). The combined organic layer was dried over sodium sulphate, filtered and concentrated to get the crude mixture. The crude was diluted with pentane and washed with acetonitrile (3×10 ml). Pentane layer was evaporated and distilled (temp. below 30° C.) to get 5-({2-[4-(dimethylamino)butyroxy]ethyl}[7-(1-octylnonyloxycarbonyl)heptyl]amino)pentyl 10-methylundecanoate (144 mg, Yield=41.4%) as a colorless liquid. ¹H-NMR (400 MHz, CDCl₃-d₃): δ 4.88-4.82 (m, 1H), 4.11 (t. J=6.4 Hz, 2H), 4.05 (t, J=6.8 Hz, 2H), 2.67 (t, J=6.4 Hz, 2H), 2.45-2.42 (m, 5H), 2.38-2.25 (m, 12H), 1.89-1.82 (m, 4H), 1.64-1.59 (m, 6H), 1.52-1.39 (m, 10H), 1.34-1.25 (br, 40H), 1.16-1.13 (m, 2H), 0.89-0.84 (m, 12H). ELSD analysis: Purity 99.71%, Calculated C₅₀H₉₉N₂O₆, [M+H]=823.74, Observed=823.65 (m/z, M+H⁺).
Example 59. Synthesis of 1-octylnonyl 6-bromohexanoate

This example illustrates synthesis of:

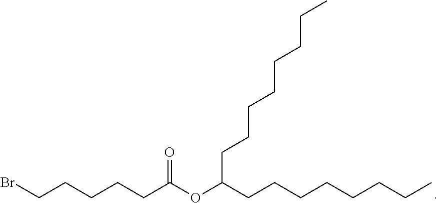 [SMILES: CCCCCCCCC(CCCCCCCC)OC(=O)CCCCCBr]

To a stirred solution of 6-bromohexanoic acid (7.61 g, 39 mmol, 2 eq) in dichloromethane (60 mL), added 4-(dimethylamino)pyridin-1-ium (4.76 g, 39 mmol, 2 eq) and {3-[cyano(ethyl)amino]propyl}dimethylazanium chloride (11.2 g, 58.5 mmol, 3 eq) and stirred for 15 min, then heptadecan-9-ol 2 (5 g, 19.5 mmol, 1.0 eq) was added. The reaction mixture was stirred at r.t. for 48 h. The progress of reaction was monitored by TLC (SM was consumed). Water (200 mL) was added to the reaction mixture and extracted with DCM (3×200 mL). The resulting organic layer was dried over Na₂SO₄, and concentrated under reduce pressure, and the crude was purified by flash column chromatography (SiO₂: 0-2% Ethyl acetate in Hexanes), to give 1-octylnonyl 6-bromohexanoate (4.6 g, Yield=53.2%) as a pale yellow liquid. ¹H-NMR (400 MHz, CDCl₃-d₃): δ 4.88-485 (m, 1H), 3.55-3.39 (m, 2H), 2.30 (t, J=9.6 Hz, 2H), 1.90-1.84 (m, 1H), 1.81-1.78 (m, 2H), 1.69-1.62 (m, 6H), 1.55-1.29 (m, 25H), 0.89-0.85 (m, 6H).
Example 60. Synthesis of 1-octylnonyl 6-(2-hydroxyethylamino)hexanoate

This example illustrates synthesis of:

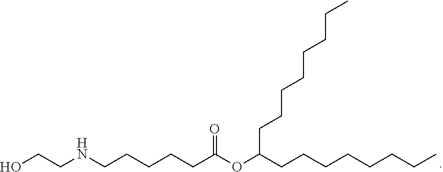 [SMILES: CCCCCCCCC(CCCCCCCC)OC(=O)CCCCCNCCO]

To a stirred solution of 1-octylnonyl 6-bromohexanoate (4.6 g, 10.6 mmol, 1 eq) in acetonitrile (40 mL), was added N-ethylbis(isopropyl)amine (5.52 mL, 31.8 mmol, 3 eq) and 2-aminoethanol (648 mg, 10.6 mmol, 1.0 eq) to it and reaction mixture was stirred at 55° C. for 16 h. The progress of reaction was monitored by TLC/ELSD (50% product form in reaction mixture by ELSD). The reaction mixture was concentrated and the crude was purified by flash column chromatography (SiO₂: 0-7% methanol in dichloromethane) to get 1-octylnonyl 6-(2-hydroxyethylamino)hexanoate (0.9 g, Yield=20.5%) as a colorless liquid. ELSD analysis: Purity 98.75%, Calculated: C₂₅H₅₂NO₃, [M+H]=414.39, Observed=414.40 (m/z, M+H⁺).

### Example 61. Synthesis of 10-(benzyloxy)-1-decanol

This example illustrates synthesis of:

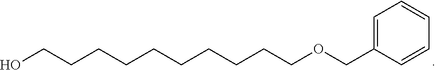 [SMILES: OCCCCCCCCCCOCc1ccccc1]

To a stirred solution of 1,10-decanediol 7 (20 g, 115 mmol, 1.0 eq) in dimethylformamide (250 mL, 3.23 mol) was added sodium hydride (4.13 g, 172 mmol, 1.5 eq) at 0° C. and stirred for 30 min at same temperature. Then (bromomethyl)benzene (13.6 mL, 115 mmol, 1.0 eq) was added to it and the reaction mixture was stirred at r.t. for 16 h. After completion of the reaction (monitored by TLC), the reaction mixture was quenched with ice-cooled water (200 ml) and extracted with ethyl acetate (3×500 ml). The organic layer was collected and dried over sodium sulphate, filtered and concentrated under reduce pressure, and the crude was purified by flash column chromatography (SiO₂: 0-8% Ethyl acetate in Hexanes), to get the product 10-(benzyloxy)-1-decanol (14 g, Yield=46%) as a colorless liquid. ¹H NMR (400 MHz, DMSO-d6): δ 7.36-7.25 (m, 5H), 4.43 (s, 2H), 4.31 (t, J=5.2 Hz, 1H), 3.42-3.32 (m, 4H), 1.55-1.48 (m, 2H), 1.40-1.37 (m, 2H), 1.29-1.24 (m, 12H).
Example 62. Synthesis of 10-(benzyloxy)decanal

This example illustrates synthesis of:

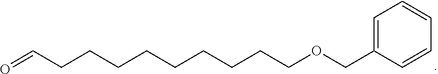 [SMILES: O=CCCCCCCCCCOCc1ccccc1]

To a stirred solution of 10-(benzyloxy)-1-decanol (13 g, 49.2 mmol, 1.0 eq) in dichloromethane (150 mL) add 1-pyridylium chloridotrioxidochromate (21.2 g, 98.3 mmol, 2 eq) at 0° C. and the reaction mixture was stirred at r.t. for 2 h. The progress of reaction was monitored by TLC (SM was consumed). The reaction mixture was diluted with pentane (500 ml) and stirred for 30 min. After 30 min, filtered through Celite bed and wash with pentane (3×200 ml). The organic layer was collected and dried over sodium sulphate, filtered and concentrated under reduce pressure, and the crude was purified by flash column chromatography (SiO₂: 0-25% Ethyl acetate in Hexanes) to give 10-(benzyloxy)decanal (11 g. Yield=77.5%) as colorless liquid. ¹H-NMR (400 MHz, CDCl₃-d3): δ 9.77 (s, 1H), 7.36-7.29 (m, 5H), 4.52 (s, 2H), 3.48 (t, J=6.8 Hz, 2H), 2.45-2.41 (m, 2H), 1.66-1.57 (m, 4H), 1.37-1.31 (m, 10H).
Example 63. Synthesis of 12-(benzyloxy)-2-methyl-2-dodecene

This example illustrates synthesis of:

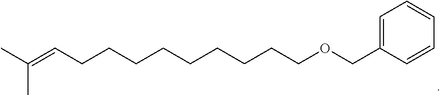 [SMILES: CC(C)=CCCCCCCCCCOCc1ccccc1]

To a stirred solution of triphenyl(propan-2-yl)phosphanium bromide (14.7 g, 38.1 mmol, 1.0 eq) in tetrahydrofuran (179 mL) was added lithium(1+) butan-1-ide (3.66 g, 57.2 mmol, 1.5 eq) dropwise at −78° C. and the reaction mixture was stirred at room temperature for 1 hr, then the mixture was cooled again to −78° C. and then 10-(benzyloxy)decanal (10 g, 38.1 mmol, 1.0 eq) (dissolved in THF) was added dropwise and the reaction mixture was stirred at room temperature for 16 h. After completion of reaction (monitored by TLC), the reaction mixture was quenched with aq. ammonium chloride solution and extracted with ethyl acetate (3×100 mL). The organic layer was collected and dried over sodium sulphate, filtered and concentrated under vacuum to get the crude. The crude product was purified through flash column chromatography (SiO₂: 0-3% Ethyl acetate in Hexane), to get 12-(benzyloxy)-2-methyl-2-dodecene (9 g, Yield=81.86%) as a colorless liquid. ¹H-NMR (400 MHz, CDCl₃-d3): δ 7.35-7.31 (m, 4H), 7.30-7.26 (m, 1H), 5.11 (t, J=7.2 Hz, 1H), 4.50 (s, 2H), 3.46 (t, J=6.8 Hz, 2H), 2.03-1.93 (m, 2H), 1.67 (s, 3H), 1.65-1.56 (m, 5H), 1.35-1.27 (m, 12H).
Example 64. Synthesis of 11-methyldodecan-1-ol

This example illustrates synthesis of:

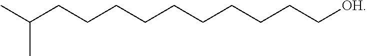

To a stirred solution of 12-(benzyloxy)-2-methyl-2-dodecene (9 g, 31.2 mmol, 1.0 eq) in methanol (45 mL) and tetrahydrofuran (45 mL) was degassed with nitrogen for 5-10 min, then Pd/C (9 g, 84.6 mmol, 2.71 eq) was added and stirred at r.t. for 16 h under Hydrogen atmosphere. After completion of reaction (SM consumed on TLC). The reaction mixture was filtered through celite bed and washed with mixture of MeOH:THF (3×200 ml). The filtrate was collected and concentrate to get the crude. The crude was purified through flash column chromatography (SiO₂: 0-10% ethyl acetate in hexane), to get 11-methyl-1-dodecanol (5 g, Yield=79.99%) as colorless liquid. ¹H NMR (400 MHz, CDCL₃-d3): δ 3.64 (q, J=6.4 Hz, 2H), 1.60-1.47 (m, 4H), 1.33-1.25 (m 15H), 0.86 (d, J=6.4 Hz, 6H).
Example 65. Synthesis of 11-methyldodecyl 6-bromohexanoate

This example illustrates synthesis of:

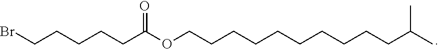 [SMILES: CC(C)CCCCCCCCCCOC(=O)CCCCCBr]

To a stirred solution of 6-bromohexanoic acid (1.95 g, 9.98 mmol, 2 eq) in dichloromethane (44.5 mL), was add 3-[cyano(ethyl)amino]propyl}dimethylazanium chloride (3.83 g, 20 mmol, 4 eq.) and 4-(dimethylamino)pyridin-1-ium (1.23 g, 9.98 mmol, 2 eq) to it. The reaction was stirred for 15 min, then 11-methyl-1-dodecanol (1 g, 4.99 mmol, 1 eq) was added. The reaction mixture was stirred at r.t. for 48 h. The progress of reaction was monitored by TLC (SM was consumed). Water (100 mL) was added to the reaction mixture and extracted with DCM (3×100 mL). The resulting organic layer was dried over Na₂SO₄, and concentrated under reduce pressure, and the crude was purified by flash column chromatography (SiO₂: 0-2% Ethyl acetate in Hexanes) to get 11-methyldodecyl 6-bromohexanoate (0.8 g, Yield=42.47%) as a colorless liquid. ¹H-NMR (400 MHz, CDCl₃-d₃): δ 4.06 (t, J=6.8 Hz, 2H), 3.54 (t, J=6.8 Hz, 1H), 3.41 (t, J=6.8 Hz, 1H), 2.32 (t, J=7.2 Hz, 2H), 1.89-1.84 (m, 1H), 1.83-1.76 (m, 1H), 1.69-1.58 (m, 4H), 1.55-1.44 (m, 3H), 1.31-1.25 (m, 14H), 1.15-1.12 (m, 2H), 0.86 (d, J=6.4 Hz, 6H).
Example 66. Synthesis of heptadecan-9-yl 6-((2-hydroxyethyl)(6-((11-methyldodecyl)oxy)-6-oxohexyl)amino)hexanoate

This example illustrates synthesis of:

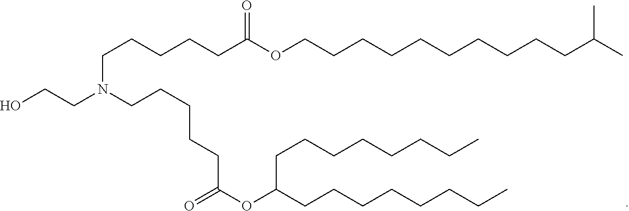 [SMILES: CCCCCCCCC(CCCCCCCC)OC(=O)CCCCCN(CCO)CCCCCC(=O)OCCCCCCCCCCC(C)C]

To a stirred solution of compound 1-octylnonyl 6-(2-hydroxyethylamino)hexanoate (0.9 g, 2.18 mmol, 1.0 eq) in acetonitrile (10.4 mL), was added N-ethylbis(isopropyl)amine (950 μL, 5.44 mmol, 2.5 eq) and 11-methyldodecyl 6-bromohexanoate (821 mg, 2.18 mmol, 1.0 eq) to it. The reaction mixture was stirred at 90° C. for 16 h. The progress of reaction was monitored by TLC/ELSD (77% product form in reaction mixture by ELSD). The reaction mixture was concentrated to get the crude mixture and the crude was purified by flash column chromatography (SiO₂: 0-4% methanol in dichloromethane), to get 1-octylnonyl 6-{(2-hydroxyethyl)[5-(11-methyldodecyloxycarbonyl)pentyl]amino}hexanoate (0.6 g, Yield=38.8%) as a colorless liquid. ¹H-NMR (400 MHz, CDCl₃-d₃): δ 4.87-4.84 (m, 1H), 4.05 (t, J=6.8 Hz, 2H), 3.78 (bs, 2H), 2.88-2.79 (m, 6H), 2.33-2.28 (q, J=5.6 Hz, 4H), 1.69-1.59 (m, 7H), 1.52-1.48 (m, 5H), 1.40-1.25 (m, 45H), 1.17-1.14 (m, 2H), 0.89-0.85 (m, 12H). ELSD analysis: Purity 99.83%, Calculated C₄₄H₈₈NO₅, [M+H]=710.67, observed 710.60 (m/z, M+H⁺).
Example 67. Synthesis of heptadecan-9-yl 6-((2-((4-(dimethylamino)butanoyl)oxy)ethyl)(6-((11-methyldodecyl)oxy)-6-oxohexyl)amino)hexanoate

This example illustrates synthesis of:

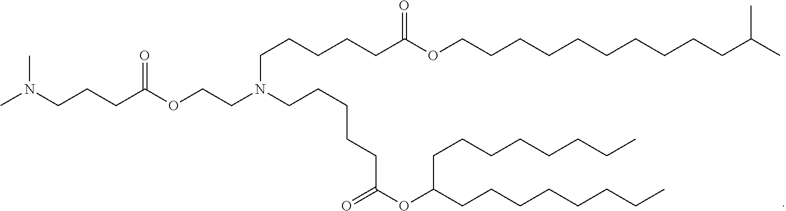 [SMILES: CCCCCCCCC(CCCCCCCC)OC(=O)CCCCCN(CCCCCC(=O)OCCCCCCCCCCC(C)C)CCOC(=O)CCCN(C)C]

To a stirred solution of compound 1-octylnonyl 6-(2-hydroxyethylamino)hexanoate (0.9 g, 2.18 mmol, 1.0 eq) in acetonitrile (10.4 mL), was added N-ethylbis(isopropyl)amine (950 μL, 5.44 mmol, 2.5 eq) and 11-methyldodecyl 6-bromohexanoate (821 mg, 2.18 mmol, 1.0 eq) to it. The reaction mixture was stirred at 90° C. for 16 h. The progress of reaction was monitored by TLC/ELSD (77% product form in reaction mixture by ELSD). The reaction mixture was concentrated to get the crude mixture and the crude was purified by flash column chromatography (SiO₂: 0-4% methanol in dichloromethane), to get 1-octylnonyl 6-{(2-hydroxyethyl)[5-(11-methyldodecyloxycarbonyl)pentyl]amino}hexanoate (0.6 g, Yield=38.8%) as a colorless liquid. ¹H-NMR (400 MHz, CDCl₃-d₃): δ 4.87-4.84 (m, 1H), 4.05 (t, J=6.8 Hz, 2H), 3.78 (bs, 2H), 2.88-2.79 (m, 6H), 2.33-2.28 (q, J=5.6 Hz, 4H), 1.69-1.59 (m, 7H), 1.52-1.48 (m, 5H), 1.40-1.25 (m, 45H), 1.17-1.14 (m, 2H), 0.89-0.85 (m, 12H). ELSD analysis: Purity 99.83%, Calculated C₄₄H₈₈NO₅, [M+H]=710.67, observed 710.60 (m/z, M+H⁺).
Example 68. Synthesis of heptadecan-9-yl 6-((2-((4-(dimethylamino)butanoyl)oxy)ethyl)(6-((11-methyldodecyl)oxy)-6-oxohexyl)amino)hexanoate

This example illustrates synthesis of:

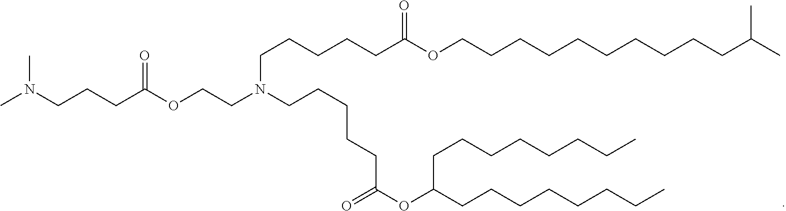 [SMILES: CCCCCCCCC(CCCCCCCC)OC(=O)CCCCCN(CCCCCC(=O)OCCCCCCCCCCC(C)C)CCOC(=O)CCCN(C)C]

To a stirred solution of 4-(dimethylamino)butyric acid (111 mg, 845 μmol, 2 eq) in dichloromethane (1.5 mL), was added {3-[cyano(ethyl)amino]propyl}dimethylazanium chloride (324 mg, 1.69 mmol, 4 eq) and 4-(dimethylamino)pyridin-1-ium (104 mg, 845 μmol, 2 eq) to it. The reaction mixture was stirred for 15 min, then 1-octylnonyl 6-{(2-hydroxyethyl)[5-(11-methyldodecyloxycarbonyl)pentyl]amino}hexanoate (0.3 g, 422 μmol, 1.0 eq) was added. The reaction mixture was stirred at r.t. for 48 h. The progress of reaction was monitored by TLC/ELSD (36.31% product form in reaction mixture with 59.64% DMAP and other Acetonitrile soluble impurity by ELSD). The reaction mixture was quenched with brine solution and extracted with DCM (10 mL×2). The combined organic layer was dried over sodium sulphate, filtered and concentrated to get the crude mixture. The crude was diluted with pentane and washed with acetonitrile (10 mL×3). Pentane layer was evaporated and distilled (temp. below 30° C.) to get 1-octylnonyl 6-({2-[4-(dimethylamino)butyroxy]ethyl}[5-(11-methyldodecyloxy carbonyl)pentyl]amino)hexanoate (195 mg, Yield=56%) as a colorless liquid. ¹H-NMR (400 MHz, CDCl₃-d₃): δ 4.89-4.83 (m, 1H), 4.10 (t, J=4 Hz, 2H), 4.05 (t, J=6.4 Hz, 2H), 2.66 (t, J=6.4 Hz, 2H), 2.44 (t, J=7.2 Hz, 4H), 2.37-2.23 (m, 8H), 2.21 (s, 6H), 1.82-1.74 (m, 2H), 1.66-1.59 (m, 5H), 1.56-1.48 (m, 4H), 1.45-1.39 (m, 4H), 1.33-1.26 (m, 44H), 1.17-1.12 (m, 2H), 0.89-0.85 (m, 12H). ELSD analysis: Purity 99.25%, Calculated: C₅₀H₉₉N₂O₆, [M+H]=823.75, Observed=823.70 (m/z, M+H⁺).
Example 69. Synthesis of undecyl 6-bromohexanoate

This example illustrates synthesis of:

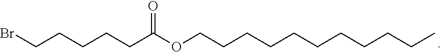 [SMILES: CCCCCCCCCCCOC(=O)CCCCCBr]

To a stirred solution of 6-bromohexanoic acid (20 g, 103 mmol, 2 eq) in dichloromethane (250 mL), was added 4-(dimethylamino)pyridin-1-ium (12.6 g, 103 mmol, 1 eq) and ({[3-(dimethylamino)propyl]imino}methylidene)(ethyl)amine hydrochloride (39.3 g, 205 mmol, 2 eq) and stirred for 15 min, then added undecan-1-ol (17.7 g, 103 mmol, 1.0 eq). The reaction mixture was stirred at r.t. for 48 h. The progress of reaction was monitored by TLC (SM was consumed). Water (500 mL) was added to the reaction mixture and extracted with DCM (3×500 mL). The resulting organic layer was dried over Na₂SO₄, and concentrated under reduce pressure, and the crude was purified by flash column chromatography (SiO₂: 0-2% Ethyl acetate in Hexanes) to give undecyl 6-bromohexanoate (13 g, Yield=36.29%) as a pale yellow liquid. H-NMR (400 MHz, CDCl₃-d3): δ 4.06 (t, J=6.8 Hz, 2H), 3.53 (t, J=6.8 Hz, 1H), 3.40 (t, J=6.8 Hz, 1H), 2.31 (t, J=7.2 Hz, 2H), 1.91-1.84 (m, 1H), 1.81-1.75 (m, 1H), 1.69-1.57 (m, 4H), 1.55-1.43 (m, 2H), 1.30-1.26 (m, 16H), 0.87 (t, J=6.8 Hz, 3H).
Example 70. Synthesis of undecyl 6-[(2-hydroxyethyl)amino]hexanoate

This example illustrates synthesis of:

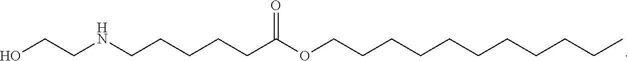 [SMILES: CCCCCCCCCCCOC(=O)CCCCCNCCO]

To a stirred solution of undecyl 6-bromohexanoate (5.72 g, 16.4 mmol, 1 eq) in ethanol (50 mL) was added ethylbis(propan-2-yl)amine (2.86 mL, 16.4 mmol, 1 eq) and 2-aminoethan-1-ol (1 g, 16.4 mmol) to it. The reaction mixture was stirred at 55° C. for 16 h. The progress of reaction was monitored by ELSD/TLC (80% product form in reaction mixture by ELSD). The reaction mixture was concentrated to get the crude mixture and the crude was purified by flash column chromatography (SiO₂: 0-7% methanol in dichloromethane), to get undecyl 6-[(2-hydroxyethyl)amino]hexanoate (2.5 g. Yield=46.34%) as a colorless semisolid compound. ELSD analysis: Purity 98.89%, Calculated: C₁₉H₄₀NO₃, [M+H]=330.30, Observed=330.35 (m/z, M+H⁺).
Example 71. Synthesis of 6-(benzyloxy)hexan-1-ol

This example illustrates synthesis of:

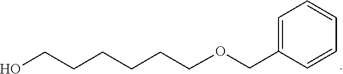 [SMILES: OCCCCCCOCc1ccccc1]

To a stirred solution of hexane-1,6-diol (10 g, 84.6 mmol, 1.0 eq) in dimethylformamide (250 ml) was added sodium hydride (4.86 g, 1.5 eq., 127 mmol) at 0° C. and stirred for 30 min at same temperature. Then (bromomethyl)benzene (14.5 g, 84.6 mmol, 1.0 eq) was added to RM and the reaction mixture was stirred at r.t. for 16 h. The progress of reaction was monitored by TLC (SM was consumed). The reaction mixture was quenched with ice-cooled water (100 ml) and extracted with ethyl acetate (3×500 ml). The organic layer was collected and dried over sodium sulphate, filtered and concentrated under reduce pressure, and the crude was purified by flash column chromatography (SiO₂: 0-8% Ethyl acetate in Hexanes), to get the product 6-(benzyloxy)hexan-1-ol (10 g, Yield=56.76%) as colorless liquid. ¹H NMR (400 MHz, CDCl₃-d₃): δ 7.34-7.27 (m, 5H), 4.50 (s, 2H), 3.63 (t, J=6.4 Hz, 2H), 3.47 (t, J=6.4 Hz, 2H), 1.65-1.61 (m, 2H), 1.59-1.54 (m, 2H), 1.45 (br, 2H), 1.40-1.38 (m, 2H).
Example 72. Synthesis of 6-(benzyloxy)hexanal

This example illustrates synthesis of:

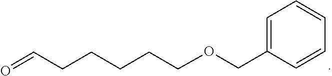 [SMILES: O=CCCCCCOCc1ccccc1]

To a stirred solution of 6-(benzyloxy)hexan-1-ol 8 (10 g, 48 mmol, 1.0 eq) in dichloromethane (150 mL) add pyridinium chlorochromate (15.5 g, 72 mmol, 1.5 eq) at 0° C. and the reaction mixture was stirred at r.t. for 2 h. The progress of reaction was monitored by TLC (SM was consumed). The reaction mixture was diluted with pentane (500 ml) and stirred for 30 min. After 30 min, mixture was filtered through celite bed and wash with pentane (3×200 ml). The organic layer was collected and dried over sodium sulphate, filtered and concentrated under reduce pressure, and the crude was purified by flash column chromatography (SiO₂: 0-25% Ethyl acetate in Hexanes), to get the product 6-(benzyloxy)hexanal (11 g, Yield=60.9%) as a colorless liquid.
Example 73. Synthesis of {[(7-methyloct-6-en-1-yl)oxy]methyl}benzene

This example illustrates synthesis of:

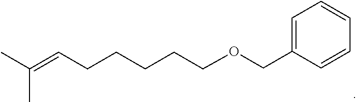 [SMILES: CC(C)=CCCCCCOCc1ccccc1]

To a stirred solution of triphenyl(propan-2-yl)phosphanium bromide (9.86 g, 25.6 mmol, 1.0 eq) in tetrahydrofuran (100 ml) was added lithium(1+) butan-1-ide (2.05 g. 32 mmol, 1.5 eq) dropwise at −78° C. The reaction mixture was stirred at room temperature for 1 hr, then the mixture was cooled again to −78° C. and then 6-(benzyloxy)hexanal (4.4 g, 21.3 mmol) (dissolved in THF) was added dropwise. The reaction mixture was stirred at room temperature for 16 h. The progress of reaction was monitored by TLC (SM was consumed). The reaction mixture was quenched with aq. ammonium chloride solution and extracted with ethyl acetate (100 mL×3) times. The organic layer was collected and dried over sodium sulphate, filtered and concentrated under vacuum to give the crude mixture. The crude product was purified through combi-flash chromatography (SiO₂: 0-3% Ethyl acetate in Hexane) to get {[(7-methyloct-6-en-1-yl)oxy]methyl}benzene (4 g. Yield=80.71%) as a colorless liquid. ¹H-NMR (400 MHz, CDCl₃-d3): δ 7.35-7.31 (m, 5H), 5.13-5.09 (m, 1H), 4.50 (m, 2H), 3.46 (t, J=6.8 Hz, 2H), 1.99-1.94 (m, 2H), 1.68 (bs, 3H), 1.64-1.59 (m, 3H), 1.36-1.29 (m, 6H).
Example 74. Synthesis of 7-methyloctan-1-ol

This example illustrates synthesis of:

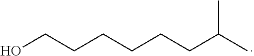

A stirred solution of {[(7-methyloct-6-en-1-yl)oxy]methyl}benzene (4 g, 17.2 mmol, 1.0 eq) in methanol (45 mL) and tetrahydrofuran (45 mL) was degassed with nitrogen for 5-10 min, then Pd/C 10%, 50% wet (4.03 g, w/w) was added and stirred at r.t. for 16 h under Hydrogen atmosphere. The progress of reaction was monitored by TLC (SM was consumed). The reaction mixture was filtered through celite bed and washed with mixture of MeOH:THF (3×200 ml). The filtrate was collected and concentrate to get the crude product which was purified through combi-flash chromatography (SiO₂: 0-10% ethyl acetate in hexane) to give 7-methyloctan-1-ol (2.2 g, Yield=88.59%) as colorless liquid. ¹H NMR (400 MHz, DMSO-d6): δ 4.32 (t, J=4.8 Hz, 1H), 3.39-3.34 (m, 2H), 1.53-1.45 (m, 1H), 1.41-1.36 (m, 2H), 1.24 (bs, 8H), 1.15-1.13 (m, 2H), 0.85 (d, J=6.4 Hz, 6H).
Example 75. Synthesis of 7-methyloctanal

This example illustrates synthesis of:

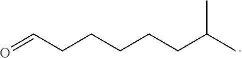

To a stirred solution of 6-(benzyloxy)hexan-1-ol (3.0 g, 20.8 mmol, 1.0 eq) in dichloromethane (150 ml), was added pyridinium chlorochromate (6.72 g, 31.2 mmol, 1.5 eq) at 0° C. and the reaction mixture was stirred at r.t. for 2 h. The progress of reaction was monitored by TLC (SM was consumed). The reaction mixture was diluted with pentane (500 ml) and stirred for 30 min. After 30 min, mixture was filtered through celite bed and wash with pentane (3×200 ml). The organic layer was collected and dried over sodium sulphate, filtered and concentrated under reduce pressure, and the crude was purified by flash column chromatography (SiO₂: 0-25% Ethyl acetate in Hexanes), to get the product 7-methyloctanal (1.5 g. Yield=50.1%) as a colorless liquid. ¹H-NMR (400 MHz, CDCl₃-d3): δ 9.76 (m, 1H), 2.44-2.39 (td, J=8.0, 2.0 Hz, 2H), 1.66-1.59 (m, 2H), 1.56-1.50 (m, 1H), 1.34-1.25 (m, 4H), 1.18-1.13 (m, 2H), 0.88-0.84 (m, 6H).
Example 76. Synthesis of 2-methylhexadecan-8-ol

This example illustrates synthesis of:

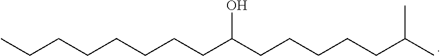 [SMILES: CCCCCCCCC(O)CCCCCC(C)C]

To a stirred solution of 7-methyloctanal (1.1 g, 7.73 mmol, 1.0 eq) in tetrahydrofuran (100 ml), was added bromo (octyl) magnesium (3.36 g, 15.5 mmol, 2.0 eq) (1M solⁿ in diethyl ether) dropwise at −78° C. The reaction mixture was stirred at room temperature for 1 hr, then reaction mixture heated up to 55° C. for 1 hr. After completion of reaction (monitored by TLC), the reaction mixture was quenched with aq. ammonium chloride solution and extracted with ethyl acetate (100 mL×3) times. The organic layer was collected and dried over sodium sulphate, filtered and concentrated under vacuum to get the crude mixture. The crude product was purified through combi-flash chromatography (SiO₂: 0-15% Ethyl acetate in Hexane), to give 2-methylhexadecan-8-ol (1.5 g, Yield=75.63%) as a colorless liquid. ¹H-NMR (400 MHz, CDCl₃-d₃): δ 3.59-3.56 (m, 1H), 1.54-1.38 (m, 6H), 1.27 (bs, 17H), 1.16-1.50 (m, 2H), 0.89-0.85 (m, 9H).
Example 77. Synthesis of 2-methylhexadecan-8-yl 8-bromooctanoate

This example illustrates synthesis of:

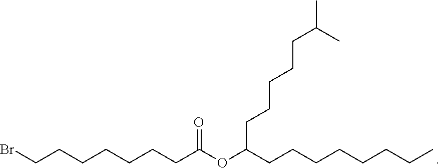 [SMILES: CCCCCCCCC(CCCCCC(C)C)OC(=O)CCCCCCCBr]

To a stirred solution of 8-bromooctanoic acid (1.04 g, 4.68 mmol, 1 eq) in dichloromethane (50 ml), was added 4-(dimethylamino)pyridin-1-ium (576 mg, 4.68 mmol, 1 eq) and {3-[cyano(ethyl)amino]propyl}dimethylazanium chloride (2.69 g. 14 mmol, 3 eq) and stirred for 15 min, then 2-methylhexadecan-8-ol (1.2 g, 4.68 mmol, 1.0 eq) was added. The reaction mixture was stirred at r.t. for 48 h. The progress of reaction was monitored by TLC (SM was consumed). Water (500 mL) was added to the reaction mixture and extracted with DCM (3×500 mL). The resulting organic layer was dried over Na₂SO₄, and concentrated under reduce pressure, and the crude was purified by flash column chromatography (SiO₂: 0-2% Ethyl acetate in Hexanes) to give 2-methylhexadecan-8-yl 8-bromooctanoate (0.8 g, Yield=36.29%) as a pale yellow liquid. ¹H-NMR (400 MHz, CDCl₃-d3): δ 4.88 (t, J=6.4 Hz, 1H), 3.55-3.41 (m, 2H), 2.29 (t, J=7.6 Hz, 2H), 1.90-1.76 (m, 2H), 1.65-1.62 (m, 2H), 1.55-1.48 (m, 5H), 1.46-1.43 (m, 2H), 1.36-1.33 (m, 4H), 1.27 (bs, 18H), 1.16-1.14 (m, 2H), 0.90-0.86 (m, 9H).
Example 78. Synthesis of 2-methylhexadecan-8-yl 8-((2-hydroxyethyl)(6-oxo-6-(undecyloxy)hexyl)amino)octanoate

This example illustrates synthesis of:

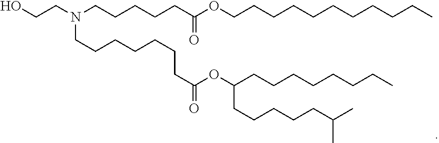 [SMILES: CCCCCCCCCCCOC(=O)CCCCCN(CCO)CCCCCCCC(=O)OC(CCCCCCCC)CCCCCC(C)C]

To a stirred solution of compound 2-methylhexadecan-8-yl 8-bromooctanoate (0.8 g, 1.73 mmol, 1.0 eq) in acetonitrile (20 mL), was added ethylbis(propan-2-yl)amine (908 μL, 5.2 mmol, 3 eq.) and undecyl 6-[(2-hydroxyethyl)amino]hexanoate (571 mg, 1.73 mmol, 1.0 eq). The reaction mixture was stirred at 95° C. for 16 h. The progress of reaction was monitored by TLC (SM was consumed). The reaction mixture was concentrated to get the crude mixture and the crude was purified by flash column chromatography (SiO₂: 0-4% methanol in dichloromethane) to give 1-octylnonyl 2-methylhexadecan-8-yl 8-[(2-hydroxyethyl)[6-oxo-6-(undecyloxy)hexyl]amino]octanoate (0.4 g, Yield=32.5%) as a pale yellow liquid. ¹H-NMR (400 MHz, CDCl₃-d3₃): δ 4.89-4.82 (m, 1H), 4.05 (t, J=6.8 Hz, 2H), 3.53-3.49 (m, 2H), 2.58 (t, J=6.8 Hz, 2H), 2.43 (q, J=7.2 Hz, 4H), 2.29 (q, J=7.2 Hz, 4H), 1.67-1.57 (m, 6H), 1.53-1.40 (m, 10H), 1.34-1.25 (m, 42H), 1.14-1.12 (m, 2H), 0.89-0.84 (m, 12H). ELSD analysis: Purity 98.89%, Calculated: C₄₄H₈₈NO₅, [M+H]=710.67, observed 710.60 (m/z, M+H).
Example 79. Synthesis of 2-methylhexadecan-8-yl 8-((2-((4-(dimethylamino)butanoyl)oxy)ethyl)(6-oxo-6-(undecyloxy)hexyl)amino)octanoate

This example illustrates synthesis of:

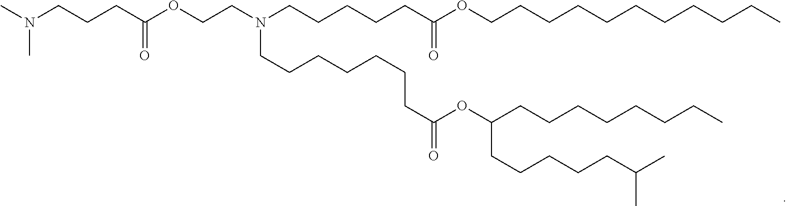 [SMILES: CCCCCCCCCCCOC(=O)CCCCCN(CCCCCCCC(=O)OC(CCCCCCCC)CCCCCC(C)C)CCOC(=O)CCCN(C)C]

To a stirred solution of 4-(dimethylamino)butyric acid (214 mg, 1.63 mmol, 4 eq) in dichloromethane (20 ml), was added {3-[cyano(ethyl)amino]propyl}dimethylazanium chloride (470 mg, 2.45 mmol, 6 eq) and 4-(dimethylamino)pyridin-1-ium (151 mg, 1.23 mmol, 3 eq). The reaction mixture was stirred for 15 min, then 1-(6-methylheptyl)nonyl 8-{(2-hydroxyethyl)[5-(undecyloxycarbonyl)pentyl]amino}octanoate (290 mg, 408 μmol, 1.0 eq) was added. The reaction mixture was stirred at r.t. for 48 h. After 48 h, TLC showed completion of reaction. The reaction mixture was quenched with brine solution and extracted with DCM (10 mL×2). The combined organic layer was dried over sodium sulphate, filtered and concentrated to get the crude mixture. The crude was diluted with pentane (25 ml) and washed with acetonitrile (10 mL×3). Pentane layer was distilled (temp. below 30° C.) under vacuum to give 1-(6-methylheptyl)nonyl 8-({2-[4-(dimethylamino)butyroxy]ethyl}[5-(undecyloxycarbonyl)pentyl]amino)octanoate (217 mg, Yield=64.54%) as a colorless liquid. ¹H-NMR (400 MHz, CDCl₃-d3): δ 4.89-4.84 (m, 1H), 4.10 (t, J=6.0 Hz, 2H), 4.05 (t, J=6.8 Hz, 2H), 2.67 (t, J=6.4 Hz, 2H), 2.46-2.41 (m, 4H), 2.36 (t, J=7.2 Hz, 2H), 2.30-2.25 (m, 8H), 2.03-2.02 (s, 2H), 1.86-1.82 (m, 2H), 1.64-1.57 (m, 6H), 1.53-1.48 (m, 4H), 1.46-1.37 (m, 4H), 1.29-1.25 (m, 45H), 1.14-1.13 (m, 2H), 0.89-0.84 (m, 12H). ELSD analysis: Purity 99.62%, Calculated: C₅₀H₉₉N₂O₆, [M+H]=823.75, observed 823.65 (m/z, M+H⁺).
Example 80. Synthesis of 9-[(tert-butyl)bis(methyl)siloxy]-1-nonanol

This example illustrates synthesis of:

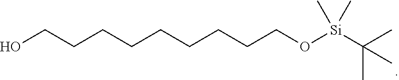 [SMILES: CC(C)(C)[Si](C)(C)OCCCCCCCCCO]

To stirred solution of 1,9-nonanediol (20 g, 125 mmol) in DCM (200 mL), was added imidazole (12.7 g, 1.5 eq., 187 mmol) and (tert-butyl)(chloro)bis(methyl)silane (15 g, 0.8 eq., 99.8 mmol) at room temperature and continued stirred for 16 h. Reaction progress was monitored by TLC and ELSD. After complete consumption of starting materials, the reaction mass was quenched with water (500 mL) and extracted with DCM (3×500 mL). The organic layer was combined and dried over sodium sulphate and evaporate under reduced pressure. The crude was purified by flash column chromatography (silica gel, 0-30% gradient of ethyl acetate in hexane) to obtain 9-[(tert-butyl)bis(methyl)siloxy]-1-nonanol (11.2 g, Yield=32.69%) as a light yellow liquid. ¹H NMR (400 MHz, CDCl₃-d3): δ 3.65-3.55 (m, 4H), 1.61-1.45 (m, 4H), 1.38-1.25 (m, 10H), 0.91 (s, 9H), 0.04 (s, 6H).
Example 81. Synthesis of 9-[(tert-butyl)bis(methyl)siloxy]nonanal

This example illustrates synthesis of:

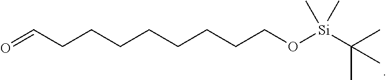 [SMILES: CC(C)(C)[Si](C)(C)OCCCCCCCCC=O]

To a stirred solution of 9-[(tert-butyl)bis(methyl)siloxy]-1-nonanol (10.4 g, 37.9 mmol) in dichloromethane (250 mL), was add Pyridinium Chlorochromate (24.5 g, 3 eq., 114 mmol) at 0° C. The reaction mixture was stirred at room temperature for 2 h. TLC showed the formation of new spots, and the starting material was consumed. The reaction mixture was diluted with pentane (500 mL) and stirred for 0.5 h. The reaction mixture was filtered through celite bed and washed with pentane (3×250 mL). The organic layer was collected and evaporated under reduced pressure. The crude was purified by flash column chromatography (silica gel, 0-25% gradient of ethyl acetate in hexane) to obtain 9-[(tert-butyl)bis(methyl)siloxy]nonanal (9 g, Yield=87.18%) as a colorless liquid. ¹H NMR (400 MHz, CDCl₃-d3): δ 9.75 (t, J=2.0 Hz, 1H), 3.58 (t, J=6.8 Hz, 2H), 2.43-2.39 (m, 2H), 1.65-1.58 (m, 2H), 1.55-1.45 (m, 2H), 1.35-1.25 (bs, 8H), 0.88 (s, 9H), 0.03 (s, 6H).
Example 82. Synthesis of (tert-butyl)bis(methyl)(10-methyl-9-undecenyloxy)silane

This example illustrates synthesis of:

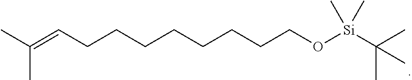 [SMILES: CC(C)=CCCCCCCCCO[Si](C)(C)C(C)(C)C]

To a stirred solution of isopropyltriphenylphosphonium bromide (15.3 g, 1.2 eq., 39.6 mmol) in tetrahydrofuran (80 mL), was added “BuLi (3.17 g. 1.5 eq., 49.5 mmol) dropwise at −78° C. and the reaction mixture was stirred at room temperature for 1 h. Then the mixture was cooled again to −78° C. and 9-[(tert-butyl)bis(methyl)siloxy]nonanal (9 g, 33 mmol) (dissolved in THF) was added dropwise. The reaction mixture was stirred at room temperature for 16 h. After completion of the reaction (monitored by TLC), the reaction mixture was quenched with aqueous ammonium chloride solution (200 mL) and extracted with ethyl acetate (3×100 mL). The organic layer was collected and dried over sodium sulphate, filtered, and concentrated under reduced pressure. The crude was purified by flash column chromatography (silica gel, 0-15% gradient of ethyl acetate in hexane) to get (tert-butyl)bis(methyl)(10-methyl-9-undecenyloxy)silane (6.2 g, Yield=62.87%) as a colorless liquid. ¹H NMR (400 MHz, DMSO-d₆): δ 5.08 (t, J=7.2 Hz, 1H), 3.55 (t, J=6.0 Hz, 2H), 1.95-1.89 (m, 2H), 1.63 (s, 3H), 1.55 (s, 3H), 1.45-1.40 (m, 2H), 1.28-1.21 (bs, 10H), 0.85 (s, 9H), 0.02 (s, 6H).
Example 83. Synthesis of 10-methyl-9-undecen-1-ol

This example illustrates synthesis of:

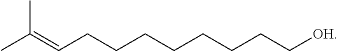

To a stirred solution of (tert-butyl)bis(methyl)(10-methyl-9-undecenyloxy)silane (4.5 g, 15.1 mmol) in tetrahydrofuran (50 mL), was added hydrogen fluoride-pyridine (1/1)(6.79 mL, 5 eq., 75.4 mmol) at 0° C. The reaction mixture was stirred at room temperature for 6 h. TLC showed the formation of new spots and reactants were consumed. The reaction mixture was quenched with saturated sodium bicarbonate and extracted with ethyl acetate (3×40 mL). The organic layer was dried over sodium sulphate, filtered, and concentrated under vacuum pressure. The crude material thus obtained was purified by flash column chromatography (silica gel, 0-5% gradient of ethyl acetate in hexane) to get a 10-methyl-9-undecen-1-ol (2.4 g, Yield=86.4%) as colorless liquid. ¹H NMR (400 MHz, CDCl₃-d3): δ 5.12-5.08 (m, 1H), 3.63 (t, J=6.4 Hz, 2H), 1.97-1.89 (m, 2H), 1.68 (s, 3H), 1.59 (s, 3H), 1.39-1.23 (m, 12H).
Example 84. Synthesis of 10-methyl-9-undecenoic acid

This example illustrates synthesis of:

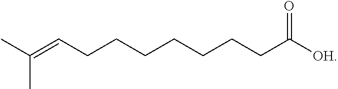

To a stirred solution of 10-methyl-9-undecen-1-ol (720 mg, 3.91 mmol) in acetone (6.34 mL) and water (1.3 mL), was added chromamethanetrione—sulfuric acid (1/1) (2.98 mL, 5 eq., 19.5 mmol) drop wise at 0° C. The reaction mixture was stirred at room temperature for 16 h. After complete consumption of starting materials (monitored by TLC), the reaction mixture was diluted with water and extracted with diethyl ether (3×100 mL). The resulting organic layer was dried over sodium sulfate, and concentrated under reduced pressure. The crude was purified by flash column chromatography (silica gel, 0-2% gradient of ethyl acetate in hexane) to get 10-methyl-9-undecenoic acid (410 mg, Yield=52.93%) as colorless liquid. ¹H NMR (400 MHz, CDCl₃-d3): δ 11.051-9.85 (bs, 1H), 5.10 (t, J=7.2 Hz, 1H), 2.34 (t, J=7.2 Hz, 2H), 1.97-1.89 (m, 2H), 1.68 (s, 3H), 1.65-1.58 (m, 2H), 1.59 (s, 3H), 1.39-1.23 (m, 8H).
Example 85. Synthesis of 7-bromoheptyl 10-methyl-9-undecenoate

This example illustrates synthesis of

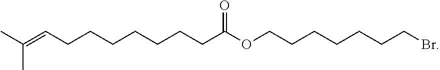 [SMILES: CC(C)=CCCCCCCCC(=O)OCCCCCCCBr]

To stirred the solution of 10-methyl-9-undecenoic acid (450 mg, 2.27 mmol) in dichloromethane (15 mL), was added N,N-dimethyl-4-pyridylamine (416 mg, 1.5 eq., 3.4 mmol) and 1-Ethyl-3-(3-dimethylaminopropyl) carbodiimide (1.31 g, 3 eq., 6.81 mmol). The reaction mixture was stirred at room temperature for 20-30 minutes. Then 7-bromo-1-heptanol (443 mg, 2.27 mmol) was added and reaction mixture was stirred at room temperature for 48 h. After complete consumption of starting materials (monitored by TLC), water (10 mL) was added to the reaction mixture and extracted with DCM (3×15 mL). The resulting organic layer was dried over sodium sulfate and concentrated under reduced pressure. The crude thus obtained was purified by flash column chromatography (silica gel, 0-2% gradient of ethyl acetate in hexane) to get 7-bromoheptyl 10-methyl-9-undecenoate (410 mg, Yield=48.13%) as a light-yellow liquid.

To stirred the solution of 10-methyl-9-undecenoic acid (450 mg. 2.27 mmol) in dichloromethane (15 mL), was added N,N-dimethyl-4-pyridylamine (416 mg, 1.5 eq., 3.4 mmol) and 1-Ethyl-3-(3-dimethylaminopropyl) carbodiimide (1.31 g, 3 eq., 6.81 mmol). The reaction mixture was stirred at room temperature for 20-30 minutes. Then 7-bromo-1-heptanol (443 mg, 2.27 mmol) was added and reaction mixture was stirred at room temperature for 48 h. After complete consumption of starting materials (monitored by TLC), water (10 mL) was added to the reaction mixture and extracted with DCM (3×15 mL). The resulting organic layer was dried over sodium sulfate and concentrated under reduced pressure. The crude thus obtained was purified by flash column chromatography (silica gel, 0-2% gradient of ethyl acetate in hexane) to get 7-bromoheptyl 10-methyl-9-undecenoate (410 mg. Yield=48.13%) as a light-yellow liquid. ¹H NMR (400 MHz, CDCl₃-d3): δ 5.12-5.07 (m, 1H), 4.05 (t, J=6.4 Hz, 2H), 3.53 (t, J=6.8 Hz, 1H), 3.40 (t, J=6.8 Hz, 1H), 2.28 (t, J=7.2 Hz, 2H), 1.97-1.89 (m, 2H), 1.89-1.71 (m, 2H), 1.68 (s, 3H), 1.65-1.53 (m, 7H), 1.48-1.38 (m, 2H), 1.35-1.21 (m, 12H).

Example 86. Synthesis of 7-((2-hydroxyethyl)(7-((2-octyldecanoyl)oxy)heptyl)amino)heptyl 10-methylundec-9-enoate

This example illustrates synthesis of:

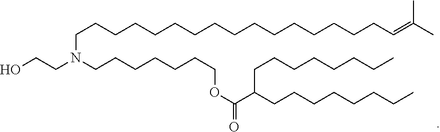 [SMILES: CCCCCCCCC(CCCCCCCC)C(=O)OCCCCCCCN(CCO)CCCCCCCCCCCCCCCCC=C(C)C]

To a stirred solution of 7-(2-hydroxyethylamino)heptyl 2-octyldecanoate (0.4 g, 906 μmol) in acetonitrile (8 mL), was added N-ethylbis(isopropyl)amine (474 μL, 3 eq., 2.72 mmol) and reaction mixture was stirred at room temperature for 10 minutes. Then, 7-bromoheptyl 10-methyl-9-undecenoate (306 mg, 0.9 eq., 815 μmol) was added and reaction mixture was stirred at 100° C. for 16 h. The progress of reaction was monitored by TLC. After starting materials were consumed, reaction mixture was concentrated under reduced pressure. The crude was purified by combi-flash chromatography (silica gel, 6-7% methanol in DCM) to obtain 7-{(2-hydroxyethyl)[7-(1-octylnonylcarbonyloxy)heptyl]amino}heptyl 10-methyl-9-undecenoate (270 mg, Yield=40%) as a colorless liquid. The compound was dissolved in dichloromethane (5 mL) under a nitrogen atmosphere and filtered through a hydrophobic PTFE syringe filter (25 mm×0.45 μm) using a glass syringe to give the desired compound. ¹H-NMR (400 MHz, CDCL₃-d3): δ 5.12-5.08 (m, 1H), 4.08-4.03 (m, 4H), 3.51 (t, J=5.6 Hz, 2H), 3.21-2.65 (bs, 1H), 2.56 (t, J=5.2 Hz, 2H), 2.43 (t, J=7.2 Hz, 4H), 2.32-2.30 (m, 1H), 2.28 (t, J=7.6 Hz, 2H), 1.95-1.91 (m, 2H), 1.68 (s, 3H), 1.63-1.52 (m, 12H), 1.47-1.37 (m, 6H), 1.32-1.20 (43H), 0.87 (t, J=6.4 Hz, 6H). ELSD analysis: Purity 99.25%, Calculated C₄₆H₉₀NO₅, [M+H]=736.68, Observed=736.75 (m/z, M+H⁺).
Example 87. Synthesis of 8-hydroxyoctyl acetate

This example illustrates synthesis of:

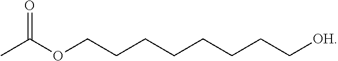

To a stirred solution of 1,8-octanediol (10 g, 68.4 mmol) in tetrahydrofuran (90.4 mL) was added of pyridine (11 mL, 137 mmol, 2 eq) at 0° C. and stirred for 30 min at same temperature. Then acetyl acetate (6.71 mL, 68.4 mmol, 1.0 eq) was added to it and the reaction mixture was stirred at r.t. for 16 h. The progress of reaction was monitored by TLC. The reaction mixture was quenched with ice cooled NaHCO₃ (250 ml) and extracted with ethyl acetate (250×3 mL). The resulting organic layer was dried over Na₂SO₄, and concentrated under reduce pressure, and the crude was purified by flash column chromatography (SiO₂: 0-15% Ethyl acetate in Hexane), to get 8-hydroxyoctyl acetate (6.3 g, Yield=48.93%) as a colorless liquid. ¹H NMR (400 MHz, CDCl3-d3): δ 4.04 (t, J=6.8 Hz, 2H), 3.63 (t, J=6.8 Hz, 2H), 2.04 (s, 3H), 1.63-1.52 (m, 4H), 1.32 (s, 8H).
Example 88. Synthesis of 8-fluorooctyl acetate

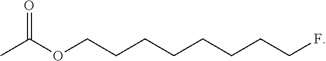

This example illustrates synthesis of: To a stirred solution of 8-hydroxyoctyl acetate (5.4 g, 28.7 mmol, 1.0 eq) in dichloromethane (54 ml), was added dropwise N,N-diethyl(trifluorothio)amine (11.4 mL, 86 mmol, 3 eq) at −78° C. The reaction mixture was stirred at r.t. for 4 h. The progress of reaction was monitored by TLC (SM was consumed). The mixture was quenched with sat. aq NaHCO₃, and extracted with DCM (3×50 mL). The resulting organic layer was dried over Na₂SO₄, and concentrated under reduce pressure, and the crude was purified by flash column chromatography (SiO₂: 0-4% Ethyl acetate in Hexane), to get 8-fluorooctyl acetate (5.1 g, 93.46%) as a pale-yellow liquid. ¹H NMR (400 MHz, CDCl₃-d3): δ 4.49 (t, J=6.0 Hz, 1H), 4.37 (t, J=6.4 Hz, 1H), 4.04 (t, J=6.8 Hz, 2H), 2.05 (s, 3H), 1.74-1.58 (m, 4H), 1.33 (s, 8H).

Example 89. Synthesis of 8-fluoro-1-octanol

This example illustrates synthesis of:

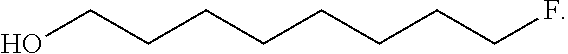

To a stirred solution of 8-fluorooctyl acetate (5 g, 26.3 mmol) in tetrahydrofuran (100 mL) was added Lithium aluminium hydride 1.0 M in THF (1.5 g, 39.4 mmol, 1.5 eq) dropwise at −78° C. and the reaction mixture was stirred at room temperature for 4 hr. After completion of reaction (monitored by TLC). The reaction mixture was quenched with aq. ammonium chloride solution and extracted with ethyl acetate (3×250 mL) times. The organic layer was collected and dried over sodium sulphate, filtered and concentrated under vacuum to get the crude mixture. The crude product was purified through combi-flash chromatography (SiO₂: 0-15% Ethyl acetate in Hexane), to get 8-fluoro-1-octanol (3.8 g, Yield=97.55%) as a colorless liquid. ¹H NMR (400 MHz, DMSO-d6): δ 4.50 (t, J=6.4 Hz, 1H), 4.38-4.32 (m, 2H), 3.39-3.35 (m, 2H), 1.69-1.56 (m, 2H), 1.42-1.27 (s, 10H).
Example 90. Synthesis of 8-fluorooctyl 8-bromooctanoate

This example illustrates synthesis of:

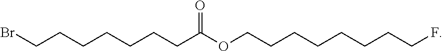 [SMILES: O=C(CCCCCCCBr)OCCCCCCCCF]

To a stirred solution of 8-bromooctanoic acid (7.53 g, 33.7 mmol, 2 eq) in dichloromethane (166 mL), was added {3-[cyano(ethyl)amino]propyl}dimethylazanium chloride (12.9 g, 67.5 mmol, 4 eq) and N,N-dimethyl-4-pyridylamine (4.12 g, 33.7 mmol, 2 eq). The reaction stirred for 15 min then 8-fluoro-1-octanol (2.5 g. 16.9 mmol) was added. The reaction mixture was stirred at r.t. for 48 h. The progress of reaction was monitored by TLC (SM was consumed). Water (250 mL) was added to the reaction mixture and extracted with DCM (3×250 mL). The resulting organic layer was dried over Na₂SO₄ and concentrated under reduce pressure to get a crude. The crude was purified by flash column chromatography (SiO₂: 0-2% Ethyl acetate in Hexanes) to get 8-fluorooctyl 8-bromooctanoate (2.0 g, Yield=33.56%) as a colorless liquid. ¹H NMR (400 MHz, CDCl₃-d3): δ 4.96 (t, J=6.4 Hz, 1H), 4.37 (t, J=6.0 Hz, 1H), 4.05 (t, J=6.8 Hz, 2H), 3.52 (t, J=6.8 Hz, 1H), 3.40 (t, J=6.8 Hz, 1H), 2.29 (t, J=7.6 Hz, 2H), 1.88-1.81 (m, 1H), 1.79-1.68 (m, 2H), 1.67-1.55 (m, 5H), 1.44-1.39 (m, 4H), 1.33-1.31 (m, 10H).
Example 91. Synthesis of 8-fluorooctyl 8-((8-(heptadecan-9-yloxy)-8-oxooctyl)(2-hydroxyethyl)amino)octanoate

This example illustrates synthesis of:

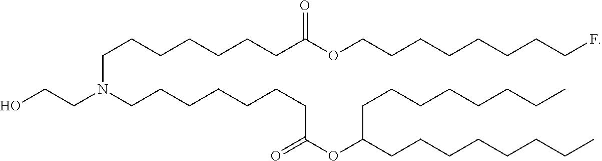 [SMILES: CCCCCCCCC(CCCCCCCC)OC(=O)CCCCCCCN(CCO)CCCCCCCC(=O)OCCCCCCCCF]

To a stirred solution of 1-octylnonyl 8-(2-hydroxyethylamino)octanoate (1.5 g, 3.4 mmol, 1.0 eq) in acetonitrile (10 mL), was added N-ethylbis(isopropyl)amine (1.32 g, 10.2 mmol 3.0 eq) and 8-fluorooctyl 8-bromooctanoate (1.32 g, 3.74 mmol, 1.1 eq). The reaction was stirred at 95° C. for 16 h. The progress of reaction was monitored by ELSD/TLC (87% product form in reaction mixture by ELSD). The reaction mixture was concentrated under reduced pressure and the crude was purified by flash column chromatography (SiO₂: 0-8% Methanol in Dichloromethane), to give 1-octylnonyl 8-{[7-(8-fluorooctyloxycarbonyl)heptyl](2-hydroxyethyl)amino}octanoate (750 mg, Yield=30.93%) as a colourless liquid. ¹H-NMR (400 MHz, CDCl₃-d3): δ 4.89-4.82 (m, 1H), 4.49 (t, J=6.4 Hz, 1H), 4.37 (t, J=6.4 Hz, 1H), 4.05 (t, J=6.8 Hz, 2H), 3.55 (bs, 2H), 2.60 (bs, 2H), 2.47 (bs, 3H), 2.30-2.25 (m, 4H), 1.73-1.67 (m, 1H), 1.64-1.59 (m, 7H), 1.41-1.25 (m, 54H), 0.89-0.85 (t, J=6.8 Hz, 6H); ¹⁹F-NMR (375 MHz, CDCL₃-d3): −218.094 ELSD analysis: Purity 99.78%, Calculated C₄₃H₈₅FNO₅, [M+H]=714.64, Observed=714.50 (m/z, M+H⁺).
Example 92. Synthesis of -fluorooctyl 8-((2-((4-(dimethylamino)butanoyl)oxy)ethyl)(8-(heptadecan-9-yloxy)-8-oxooctyl)amino)octanoate

This example illustrates synthesis of:

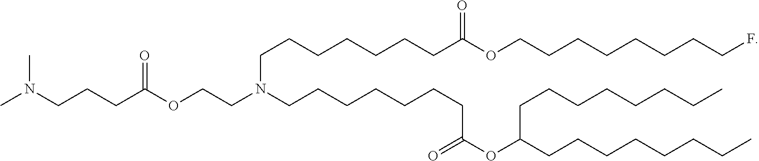 [SMILES: CCCCCCCCC(CCCCCCCC)OC(=O)CCCCCCCN(CCCCCCCC(=O)OCCCCCCCCF)CCOC(=O)CCCN(C)C]

To a stirred solution of 4-(dimethylamino)butyric acid (184 mg, 4 eq., 1.4 mmol) in dichloromethane (22.5 mL) was added {3-[cyano(ethyl)amino]propyl}dimethylazanium chloride (201 mg, 3 eq., 1.05 mmol) and N,N-dimethyl-4-pyridylamine (171 mg, 4 eq., 1.4 mmol) and stirred for 15 min, then 1-octylnonyl 8-{[7-(8-fluorooctyloxycarbonyl)heptyl](2-hydroxyethyl)amino}octanoate (250 mg, 350 μmol) was added. The reaction mixture was stirred at r.t. for 16 h. The progress of reaction was monitored by ELSD/TLC (62.24% product form in reaction mixture with 33% DMAP by ELSD). The reaction mixture was quenched with brine solution and extracted with DCM (2×100 mL). The combined organic layer was dried over sodium sulphate, filtered and concentrated to get the crude mixture. The crude was diluted with pentane and washed with acetonitrile (3×100 ml). Pentane layer was evaporated and distilled (temp. below 30° C.) to give 1-octylnonyl 8-({2-[4-(dimethylamino)butyroxy]ethyl}[7-(8-fluorooctyloxycarbonyl)heptyl]amino)octanoate (142 mg, Yield=49.03%) as colorless liquid. ¹H-NMR (400 MHz, CDCl₃-d3)-δ 4.87-4.84 (m, 1H), 4.49 (t, J=6.4 Hz, 1H), 4.37 (t, J=6.0 Hz, 1H), 4.10 (t, J=6.4 Hz, 2H), 4.05 (t, J=6.8 Hz, 2H), 2.66 (t, J=6.4 Hz, 2H), 2.44-2.40 (m, 4H), 2.33 (t, J=6.8 Hz, 2H), 2.30-2.25 (m, 6H), 2.20 (m, 6H), 1.81-1.75 (m, 2H), 1.73-1.67 (m, 1H), 1.62-1.59 (m, 7H), 1.50-1.49 (m, 4H), 1.44-1.38 (m, 6H), 1.37-1.32 (m, 18H), 1.25 (br, 25H), 0.87 (t, J=6.0 Hz, 6H). ¹⁹F-NMR (375 MHz, CDCl₃-d3): −218.082. ELSD analysis: Purity 99.93%, Calculated C₄₉H₉₆FN₂O₆, [M+H]=827.73, Observed=827.65 (m/z, M+H⁺).
Example 93. Synthesis of nonyl 8-bromooctanoate

This example illustrates synthesis of:

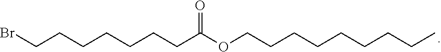 [SMILES: CCCCCCCCCOC(=O)CCCCCCCBr]

To a stirred solution of 8-bromooctanoic acid (5 g, 22.4 mmol)₂ eq) in dichloromethane (250 mL), was added N,N-dimethyl-4-pyridylamine (4.11 g, 33.6 mmol, 1.5 eq) and ({[3-(dimethylamino)propyl]imino}methylidene)(ethyl)amine hydrochloride (12.9 g, 67.2 mmol, 3 eq) and stirred for 15 min, then added 1-nonanol (3.23 g, 22.4 mmol). The reaction mixture was stirred at r.t. for 48 h. The progress of reaction was monitored by TLC (SM was consumed). Water (500 mL) was added to the reaction mixture, and extracted with DCM (3×500 mL). The resulting organic layer was dried over Na₂SO₄, and concentrated under reduce pressure, and the crude was purified by flash column chromatography (SiO₂: 0-2% Ethyl acetate in Hexanes) to give nonyl 8-bromooctanoate (4 g, Yield=51.09%) as a pale yellow liquid. ¹H-NMR (400 MHz, CDCl₃-d3): δ 4.05 (t, J=6.8 Hz, 2H), 3.54-3.38 (m, 2H), 2.29 (t, J=7.6 Hz, 2H), 1.86-1.72 (m, 2H), 1.63-1.57 (m, 4H), 1.44-1.41 (m, 2H), 1.39-1.26 (m, 16H), 0.87-0.85 (m, 3H).
Example 94. Synthesis of nonyl 8-(2-hydroxyethylamino)octanoate

This example illustrates synthesis of:

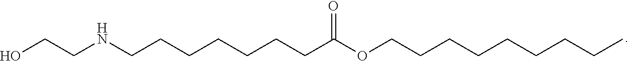 [SMILES: CCCCCCCCCOC(=O)CCCCCCCNCCO]

To a stirred solution of nonyl 8-bromooctanoate (2 g, 5.72 mmol, 1.0 eq) in acetonitrile (20 mL), was added N-ethylbis(isopropyl)amine (1.2 mL, 6.87 mmol, 1.2 eq) and 2-aminoethanol (350 mg, 5.72 mmol, 1.0 eq) to it and reaction mixture was stirred at 55° C. for 16 h. The progress of reaction was monitored by TLC (50% product form in reaction mixture by ELSD). The reaction mixture was concentrated to get the crude mixture and the crude was purified by flash column chromatography (SiO₂: 0-7% methanol in dichloromethane), to give nonyl 8-(2-hydroxyethylamino)octanoate (0.9 g, Yield=47.71%) as a colorless liquid compound. ¹H-NMR (400 MHz, CDCl₃-d3): δ 4.05-4.02 (m, 4H), 3.20-3.18 (m, 2H), 3.03 (t, J=8.0 Hz, 2H), 2.28 (t, J=7.6 Hz, 2H), 1.92-1.86 (m, 2H), 1.63-1.52 (m, 5H), 1.40-1.26 (m, 18H), 0.89-0.85 (t, J=6.8 Hz, 3H).
Example 95. Synthesis of 1-fluoroheptadecan-9-yl 8-((2-hydroxyethyl)(8-(nonyloxy)-8-oxooctyl)amino)octanoate

This example illustrates synthesis of:

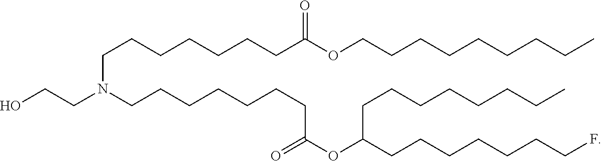 [SMILES: CCCCCCCCCOC(=O)CCCCCCCN(CCO)CCCCCCCC(=O)OC(CCCCCCCC)CCCCCCCF]

To a stirred solution of nonyl 8-(2-hydroxyethylamino)octanoate (0.5 g, 1.52 mmol, 1.0 eq) in acetonitrile (10 mL), was added N-ethylbis(isopropyl)amine (409 μL, 2.28 mmol, 1.5 eq) and 9-fluoro-1-octylnonyl 8-bromooctanoate (728 mg, 1.52 mmol, 1.0 eq) to it. The reaction was stirred at 95° C. for 16 h. The progress of reaction was monitored by ELSD/TLC (71% product formation in reaction mixture by ELSD). The reaction mixture was concentrated under reduced pressure and the crude was purified by flash column chromatography (SiO₂: 0-8% Methanol in Dichloromethane), to give 9-fluoro-1-octylnonyl 8-{(2-hydroxyethyl)[7-(nonyloxycarbonyl)heptyl]amino}octanoate (123 mg, Yield=11.13%) as a colorless liquid. ¹H-NMR (400 MHz, CDCl₃-d3): δ 4.86-4.79 (m, 1H), 4.47-4.32 (m, 2H), 4.02 (t, J=6.8 Hz, 2H), 3.48 (t, J=5.2 Hz, 2H), 2.99 (bs, 1H), 2.53 (t, J=5.2 Hz, 2H), 2.39 (t, J=7.2 Hz, 4H), 2.27-2.22 (m, 4H), 1.99 (br, 1H), 1.69-1.54 (m, 8H), 1.47-1.40 (m, 4H), 1.39-1.22 (m, 49H), 0.86-0.83 (m, 6H); ¹⁹F-NMR (375 MHz, CDCL₃-d3): −217.99 ELSD analysis: Purity 99.81%, Calculated C₄₄H₈₇FNO₅, [M+H]=728.66, Observed=728.60 (m/z, M+H⁺).
Example 96. Synthesis of undecyl 6-bromohexanoate

This example illustrates synthesis of:

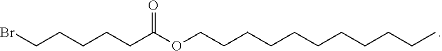 [SMILES: CCCCCCCCCCCOC(=O)CCCCCBr]

To a stirred solution of 6-bromohexanoic acid (10 g, 51.3 mmol, 1.0 eq.) in dichloromethane (150 mL), was added 4-(dimethylamino)pyridin-1-ium (6.26 g, 51.3 mmol, 1.0 eq.) and ({[3-(dimethylamino)propyl]imino}methylidene)(ethyl)amine hydrochloride (19.7 g, 103 mmol, 2.0 eq.) and stirred for 15 minutes. Then, undecan-1-ol 2 (8.83 g, 51.3 mmol, 1.0 eq.) was added and reaction mixture was stirred at room temperature for 48 h. The progress of reaction was monitored by TLC. After complete consumption of starting materials (monitored by TLC), water (500 mL) was added to the reaction mixture and extracted with DCM (3×250 mL). The resulting organic layer was dried over sodium sulfate and concentrated under reduce pressure. The crude was purified by flash column chromatography (silica gel, 0-10% gradient of ethyl acetate in hexane) to get undecyl 6-bromohexanoate (5.6 g, Yield=31.27%) as a colorless liquid. ¹H-NMR (400 MHz, CDCl₃-d3): δ 4.05 (t, J=6.8 Hz, 2H), 3.53 (t, J=6.8 Hz, 2H), 2.31 (t, J=7.6 Hz, 2H), 1.89-1.75 (m, 2H), 1.69-1.56 (m, 4H), 1.51-1.43 (m, 2H), 1.30-1.25 (m, 16H), 0.87 (t, J=6.8 Hz, 3H).
Example 97. Synthesis of undecyl 6-(2-hydroxyethylamino)hexanoate

This example illustrates synthesis of:

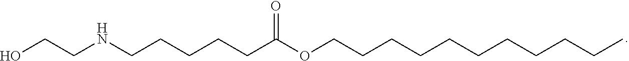 [SMILES: CCCCCCCCCCCOC(=O)CCCCCNCCO]

To a stirred solution of undecyl 6-bromohexanoate (5.72 g, 16.4 mmol) in acetonitrile (20 mL) was added N-ethylbis(isopropyl)amine (6.35 g, 49.1 mmol, 3.0 eq.) and 2-amino-1-ethanol (1.0 g, 16.4 mmol, 1.0 eq.). The reaction mixture was stirred at 55° C. for 16 h. The progress of reaction was monitored by ELSD and TLC. The ELSD of reaction mixture showed 53% of desired product mass. The reaction mixture was concentrated and crude thus obtained was purified by flash column chromatography (silica gel, 0-7% gradient of methanol in dichloromethane) to get undecyl 6-(2-hydroxyethylamino)hexanoate (1.7 g, Yield=31.51%) as a colorless liquid. ELSD analysis: Purity 99.03%, Calculated: C₁₉H₄₀NO₃, [M+H]=330.18, Observed=330.25 (m/z, M+H⁺).
Example 98. Synthesis of 8-hydroxyoctyl acetate

This example illustrates synthesis of:

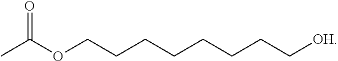

To a stirred solution of 1,8-octanediol (25 g, 171 mmol, 1.0 eq.) in tetrahydrofuran (250 mL), pyridine (27.6 mL, 342 mmol, 2.0 eq.) was added at 0° C. and stirred for 30 minutes at same temperature. Then acetyl acetate (16.8 mL, 171 mmol, 1.0 eq.) was added and the reaction mixture was stirred at room temperature for 16 h. After complete consumption of starting materials (monitored by TLC), the reaction mixture was quenched with ice-cooled sodium bicarbonate (800 mL) and extracted with ethyl acetate (3×500 mL). The resulting organic layer was dried over sodium sulfate and concentrated under reduce pressure. The crude material thus obtained was purified by flash column chromatography (silica gel, 0-50% ethyl acetate in hexane) to get 8-hydroxyoctyl acetate (20 g, Yield=62.14%) as a colorless liquid. ¹H NMR (400 MHz, DMSO-d6): δ 4.31 (t, J=5.2 Hz, 1H), 3.97 (t, J=6.8 Hz, 2H), 3.67 (q, J=6.8 Hz, 2H), 1.99 (s, 3H), 1.56-1.51 (m, 2H), 1.41-1.38 (m, 2H), 1.26 (bs, 8H).
Example 99. Synthesis of 8-fluorooctyl acetate

This example illustrates synthesis of:

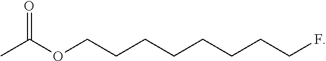

To a stirred solution of 8-hydroxyoctyl acetate (20 g, 106 mmol, 1.0 eq.) in dichloromethane (200 mL), was added dropwise N,N-diethyl(trifluorothio)amine (28.1 mL, 212 mmol, 2.0 eq.) at −78° C. The reaction mixture was allowed to warm to room temperature and stirring was continued for 4 h. The progress of reaction was monitored by TLC. After complete consumption of starting materials, reaction mixture was quenched with saturated aqueous sodium bicarbonate (100 mL) and extracted with DCM (3×200 mL). The resulting organic layer was dried over sodium sulfate and concentrated under reduce pressure. The crude thus obtained was purified by flash column chromatography (silica gel, 0-30% gradient of ethyl acetate in hexane) to get 8-fluorooctyl acetate (16 g. Yield=79.16%) as a yellow oil. ¹H NMR (400 MHz, CDCl₃-d3): δ 4.51 (t, J=6.0 Hz, 1H), 4.39 (t, J=6.0 Hz, 1H), 4.06 (t, J=6.8 Hz, 2H), 2.03 (s, 3H), 1.76-1.59 (m, 4H), 1.49-1.31 (bs, 8H).
Example 100. Synthesis of 8-fluorooctyl acetate

This example illustrates synthesis of:

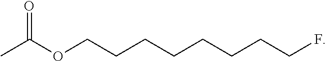

To a stirred solution of 8-hydroxyoctyl acetate (20 g, 106 mmol, 1.0 eq.) in dichloromethane (200 mL), was added dropwise N,N-diethyl(trifluorothio)amine (28.1 mL, 212 mmol, 2.0 eq.) at −78° C. The reaction mixture was allowed to warm to room temperature and stirring was continued for 4 h. The progress of reaction was monitored by TLC. After complete consumption of starting materials, reaction mixture was quenched with saturated aqueous sodium bicarbonate (100 mL) and extracted with DCM (3×200 mL). The resulting organic layer was dried over sodium sulfate and concentrated under reduce pressure. The crude thus obtained was purified by flash column chromatography (silica gel, 0-30% gradient of ethyl acetate in hexane) to get 8-fluorooctyl acetate (16 g, Yield=79.16%) as a yellow oil. ¹H NMR (400 MHz, CDCl₃-d3): δ 4.51 (t, J=6.0 Hz, 1H), 4.39 (t, J=6.0 Hz, 1H), 4.06 (t, J=6.8 Hz, 2H), 2.03 (s, 3H), 1.76-1.59 (m, 4H), 1.49-1.31 (bs, 8H).
Example 101. Synthesis of 8-fluoro-1-octanol

This example illustrates synthesis of:

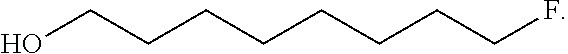

To a stirred solution of 8-fluorooctyl acetate (12 g, 63.1 mmol, 1.0 eq.) in tetrahydrofuran (200 mL) was added lithium aluminium hydride 1.0 M in THF (3.59 g, 94.6 mmol, 1.5 eq.) dropwise at −78° C. The reaction mixture was allowed to warm to room temperature and stirring was continued for 4 h. After complete consumption of starting materials (monitored by TLC), the reaction mixture was quenched with aqueous ammonium chloride solution (50 mL) and extracted with ethyl acetate (3×250 mL). The organic layer was collected and dried over sodium sulphate, filtered and concentrated under vacuum to get the crude mixture. The crude product was purified by flash column chromatography (silica gel, 0-15% gradient of ethyl acetate in hexane) to get 8-fluoro-1-octanol (6.0 g. Yield=96.2%) as a colorless liquid. ¹H NMR (400 MHz, DMSO-d6): δ 4.48 (t, J=6.0 Hz, 1H), 4.38-4.31 (m, 2H), 3.39 (m, 2H), 1.69-1.56 (m, 2H), 1.59-1.32 (m, 10H).
Example 102. Synthesis of 8-fluorooctanal

This example illustrates synthesis of:

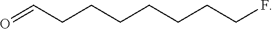

To a stirred solution of 8-fluoro-1-octanol (9.0 g, 60.7 mmol, 1.0 eq.) in dichloromethane (200 mL), added pyridinium chlorochromate (19.6 g, 91.1 mmol, 1.5 eq.) at 0° C. and the reaction mixture was stirred at room temperature for 2 h. The progress of reaction was monitored by TLC. After complete consumption of starting materials, reaction mixture was diluted with pentane (750 mL) and stirred for 30 minutes. The reaction mixture was filtered through celite bed and wash with pentane (3×500 mL). The organic layer was collected and dried over sodium sulphate, filtered and concentrated under reduce pressure. The crude material thus obtained was purified by flash column chromatography (silica gel, 0-25% gradient of ethyl acetate in hexanes) to get 8-fluorooctanal (6.0 g, Yield=67.59%) as colorless liquid. ¹H-NMR (400 MHz, CDCl₃-d3): δ 9.78 (t, J=1.6 Hz, 1H), 4.51 (t, J=6.0 Hz, 1H), 4.39 (t, J=6.0 Hz, 1H), 2.47-2.44 (m, 1H), 1.76-1.62 (m, 4H), 1.44-1.35 (bs, 6H).
Example 103. Synthesis of 1-fluoro-8-hexadecanol

This example illustrates synthesis of:

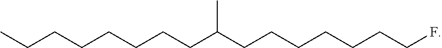 [SMILES: CCCCCCCCC(C)CCCCCCCF]

To a stirred solution of 8-fluorooctanal (6.0 g, 41 mmol, 1.0 eq.) in tetrahydrofuran (50 mL), was added bromo (octyl) magnesium (10.7 g, 49.2 mmol, 1.2 eq.)(1M solution in diethyl ether) dropwise at −78° C. The reaction mixture was allowed to warm to room temperature and stirring was continued for 1 h. Then, reaction mixture heated up to 55° C. for 1 h. After complete consumption of starting materials (monitored by TLC), reaction mixture was quenched with aqueous ammonium chloride solution (30 mL) and extracted with ethyl acetate (3×100 mL). The organic layer was collected and dried over sodium sulphate, filtered and concentrated under vacuum. The crude product was purified by flash column chromatography (silica gel, 0-15% gradient of ethyl acetate in hexane) to get 1-fluoro-8-hexadecanol (6.0 g, Yield=56.14%) as a white solid. ¹H-NMR (400 MHz, CDCl₃-d3): δ 4.70 (t, J=6.0 Hz, 1H), 4.35 (t, J=6.0 Hz, 1H), 4.21 (d, J=5.6 Hz, 1H), 1.68-1.55 (m, 2H), 1.46-1.27 (m, 24H), 0.89-0.85 (m, 3H).
Example 104. Synthesis of 1-(7-fluoroheptyl)nonyl 8-bromooctanoate

This example illustrates synthesis of:

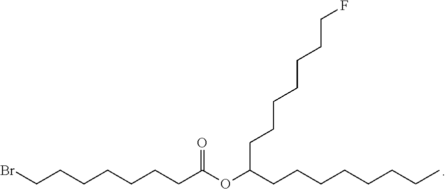 [SMILES: CCCCCCCCC(CCCCCCCF)OC(=O)CCCCCCCBr]

To a stirred solution of 8-bromooctanoic acid (5.0 g, 22.4 mmol, 1.0 eq.) in dichloromethane (100 mL) was added ({[3-(dimethylamino)propyl]imino}methylidene)(ethyl)amine hydrochloride (8.59 g, 44.8 mmol, 2.0 eq.) and N,N-dimethyl-4-pyridylamine (2.74 g, 22.4 mmol, 1.0 eq.). The reaction mixture was stirred for 15 minutes then added 1-fluoro-8-hexadecanol (5.84 g, 22.4 mmol, 1.0 eq.) and stirred at room temperature for 48 h. The progress of reaction was monitored by TLC. After complete consumption of starting materials, water (250 mL) was added to the reaction mixture and extracted with DCM (3×250 mL). The resulting organic layer was dried over sodium sulfate and concentrated under reduce pressure to get a crude. The crude was purified by flash column chromatography (silica gel, 0-2% ethyl acetate in hexanes) to get 1-(7-fluoroheptyl)nonyl 8-bromooctanoate (4.8 g, Yield=46.01%) as a colorless liquid. ¹H NMR (400 MHz, CDCL₃-d3): δ 4.86 (quin, J=6.4 Hz, 1H), 4.48 (t, J=6.0 Hz, 1H), 4.37 (t, J=6.0 Hz, 1H), 3.54-3.38 (m, 2H), 2.28 (t, J=7.6 Hz, 2H), 1.88-1.81 (m, 2H), 1.78-1.57 (m, 4H), 1.45-1.41 (m, 4H), 1.34-1.26 (m, 26H), 0.87 (t, J=6.8 Hz, 3H).
Example 105. Synthesis of 1-fluorohexadecan-8-yl 8-((2-hydroxyethyl)(6-oxo-6-(undecyloxy)hexyl)amino)octanoate

This example illustrates synthesis of:

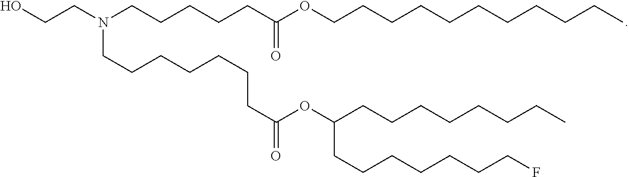 [SMILES: CCCCCCCCCCCOC(=O)CCCCCN(CCO)CCCCCCCC(=O)OC(CCCCCCCC)CCCCCCCF]

To a stirred solution of undecyl 6-(2-hydroxyethylamino)hexanoate (0.5 g, 1.52 mmol) in acetonitrile (10 mL), was added N-ethylbis(isopropyl)amine (0.793 μL, 3 eq., 4.55 mmol) and 1-(7-fluoroheptyl)nonyl 8-bromooctanoate (706 mg, 1.52 mmol). The reaction was stirred at 95° C. for 16 h. The progress of reaction was monitored by TLC and ELSD. The reaction mixture was concentrated under reduced pressure and the crude was purified by flash column chromatography (silica gel, 0-7% gradient of methanol in dichloromethane) to get 1-(7-fluoroheptyl)nonyl 8-{(2-hydroxyethyl)[5-(undecyloxycarbonyl)pentyl]amino}octanoate (170 mg) as a colorless liquid. The compound (170 mg) was obtained after column chromatography was dissolved in dichloromethane (10 mL) and added anhydrous potassium carbonate (5 eq.) under nitrogen atmosphere and stirred vigorously for 2 h. Then, reaction mixture was filtered through hydrophobic PTFE syringe filter (25 mm×0.45 μm) using glass syringe to get 1-(7-fluoroheptyl)nonyl 8-{(2-hydroxyethyl)[5-(undecyloxycarbonyl)pentyl]amino}octanoate (152 mg, Yield=14.03%) as a colorless liquid. ¹H-NMR (400 MHz, CDCl₃-d3): δ 4.86 (quin, J=6.4 Hz, 1H), 4.48 (t, J=6.0 Hz, 1H), 4.36 (t, J=6.0 Hz, 1H), 4.05 (t, J=6.0 Hz, 2H), 3.52-3.50 (m, 2H), 3.10-2.87 (bs, 1H), 2.55 (t, J=5.2 Hz, 2H), 2.45-2.40 (m, 4H), 2.28 (q, J=8.0 Hz, 4H), 1.72-1.57 (m, 9H), 1.50-1.40 (m, 9H), 1.30-1.25 (m, 42H), 0.89-0.86 (m, 6H). ¹⁹F-NMR (375 MHz, CDCL₃-d3): δ −218.05 ELSD analysis: Purity 99.10%, Calculated C₄₃H₈₅FNO₅, [M+H]=714.63, Observed=714.75 (m/z, M+H⁺).
Example 106. Synthesis of nonyl 6-bromohexanoate

This example illustrates synthesis of:

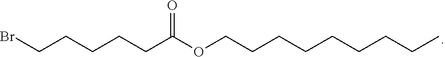 [SMILES: CCCCCCCCCOC(=O)CCCCCBr]

To a stirred solution of 6-bromohexanoic acid (10 g, 51.3 mmol, 1.0 eq.) in dichloromethane (150 mL), was added N,N-dimethyl-4-pyridylamine (6.26 g, 51.3 mmol, 1.0 eq.) and ({[3-(dimethylamino)propyl]imino}methylidene)(ethyl)amine hydrochloride (19.7 g, 103 mmol, 2.0 eq.) and stirred for 15 minutes. Then, 1-nonanol (7.4 g, 51.3 mmol, 1.0 eq.) was added and reaction mixture was stirred at room temperature for 48 h. After complete consumption of starting materials (monitored by TLC), water (500 mL) was added to the reaction mixture and extracted with DCM (3×500 mL). The resulting organic layer was dried over sodium sulfate and concentrated under reduce pressure. The crude material thus obtained was purified by flash column chromatography (silica gel, 0-10% ethyl acetate in hexanes) to get nonyl 6-bromohexanoate (5.6 g, Yield=34%) as a colorless liquid. ¹H-NMR (400 MHz, CDCl₃-d3): δ 4.05 (t, J=6.8 Hz, 2H), 3.53 (t, J=6.8 Hz, 2H), 2.31 (t, J=7.6 Hz, 2H), 1.91-1.75 (m, 2H), 1.69-1.58 (m, 4H), 1.50-1.40 (m, 2H), 1.34-1.26 (m, 12H), 0.87 (t, J=6.8 Hz, 3H).
Example 107. Synthesis of nonyl 6-(2-hydroxyethylamino)hexanoate

This example illustrates synthesis of:

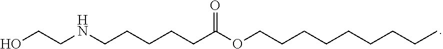 [SMILES: CCCCCCCCCOC(=O)CCCCCNCCO]

To a stirred solution of nonyl 6-bromohexanoate (5.26 g, 16.4 mmol, 1.0 eq.) in acetonitrile (20 mL), was added N-ethylbis(isopropyl)amine (6.35 g, 49.1 mmol, 3.0 eq.) and 2-amino-1-ethanol (1.0 g. 16.4 mmol, 1.0 eq.). Reaction mixture was stirred at 55° C. for 16 h. The progress of reaction was monitored by TLC. ELSD of reaction mixture showed 53% of desired product mass. The reaction mixture was concentrated and crude was purified by flash column chromatography (silica gel, 0-7% gradient of methanol in dichloromethane) to get nonyl 6-(2-hydroxyethylamino)hexanoate (1.7 g. Yield=34.45%) as a colorless liquid. ¹H-NMR (400 MHz, CDCl₃-d3): δ 4.07 (t, J=6.8 Hz, 2H), 3.77 (t, J=5.2 Hz, 2H), 2.91 (t, J=5.2 Hz, 2H), 2.77 (t, J=7.6 Hz, 2H), 2.33 (t, J=7.6 Hz, 2H), 1.68-1.61 (m, 7H), 1.42-1.36 (m, 2H), 1.28 (m, 12H), 0.89 (t, J=6.8 Hz, 3H) ELSD analysis: Purity 99.96%, Calculated: C₁₇H₃₆NO₃, [M+H]=302.27, Observed=302.35 (m/z, M+H⁺).
Example 108. Synthesis of 1-fluorohexadecan-8-yl 8-((2-hydroxyethyl)(6-(nonyloxy)-6-oxohexyl)amino)octanoate

This example illustrates synthesis of:

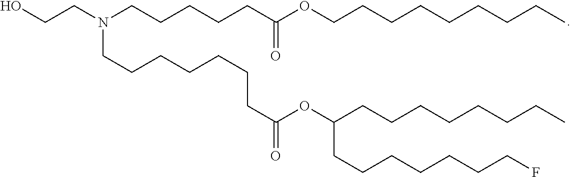 [SMILES: CCCCCCCCCOC(=O)CCCCCN(CCO)CCCCCCCC(=O)OC(CCCCCCCC)CCCCCCCF]

To a stirred solution of nonyl 6-(2-hydroxyethylamino)hexanoate (0.4 g, 1.33 mmol) in acetonitrile (10 mL), was added N-ethylbis(isopropyl)amine (0.693 μL, 3 eq., 3.98 mmol) and 1-(7-fluoroheptyl)nonyl 8-bromooctanoate (618 mg, 1.33 mmol). The reaction was stirred at 95° C. for 16 h. The progress of reaction was monitored by ELSD and TLC. The reaction mixture was concentrated under reduced pressure and the crude was purified by flash column chromatography (silica gel, 0-8% gradient of methanol in DCM) to get 1-(7-fluoroheptyl)nonyl 8-{(2-hydroxyethyl)[5-(nonyloxycarbonyl)pentyl]amino}octanoate (230 mg) as a pale-yellow liquid. The compound (230 mg) obtain after column chromatography was dissolved in dichloromethane (10 mL) and added anhydrous potassium carbonate (5 eq.) under nitrogen atmosphere and stirred vigorously for 2 h. Then, reaction mixture was filtered through hydrophobic PTFE syringe filter (25 mm×0.45 μm) using glass syringe to get 1-(7-fluoroheptyl)nonyl 8-{(2-hydroxyethyl)[5-(nonyloxycarbonyl)pentyl]amino}octanoate (210 mg, Yield=23.07%) as a pale yellow liquid. ¹H-NMR (400 MHz, CDCl₃-d3): δ 4.86 (quin, J=6.4 Hz, 1H), 4.36 (t, J=6.0 Hz, 1H), 4.48 (t, J=6.0 Hz, 1H), 4.05 (t, J=6.0 Hz, 2H), 3.50 (t, J=5.2 Hz, 2H), 2.56 (t, J=5.2 Hz, 2H), 2.46-2.41 (m, 4H), 2.28 (q, J=8.0 Hz, 4H), 1.72-1.67 (m, 1H), 1.66-1.57 (m, 7H), 1.50-1.47 (m, 9H), 1.37-1.25 (m, 40H), 0.89-0.86 (m, 6H). ¹⁹F-NMR (375 MHz, CDCL₃-d3): δ −218.03 ELSD analysis: Purity 98.62%, Calculated C₄₁H₈₁FNO₅, [M+H]=686.61, Observed=686.40 (m/z, M+H⁺).
Example 109. Synthesis of 6-(benzyloxy)hexan-1-ol

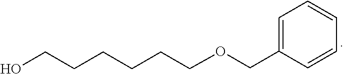 [SMILES: OCCCCCCOCc1ccccc1]

This example illustrates synthesis of: To a stirred solution of hexane-1,6-diol (20 g, 169 mmol, 1.0 eq.) in dimethylformamide (250 mL) was added sodium hydride (7.78 g, 203 mmol, 1.2 eq.) at 0° C. and stirred for 30 minutes at 0° C. Then (bromomethyl)benzene (20.2 mL, 169 mmol, 1.0 eq.) was added and the reaction mixture was stirred at room temperature for 16 h. The progress of reaction was monitored by TLC. The reaction mixture was quenched with ice-cooled water (100 mL) and extracted with ethyl acetate (3×500 mL). The organic layer was collected and dried over sodium sulphate, filtered and concentrated under reduce pressure. The crude was purified by flash column chromatography (silica gel, 0-8% gradient of ethyl acetate in hexane) to get 6-(benzyloxy)hexan-1-ol 8 (13 g, Yield=36.88%) as a colorless liquid. ¹H NMR (400 MHz, CDCl₃-d3): δ 7.39-7.30 (m, 5H), 4.52 (s, 2H), 3.66 (t, J=6.8 Hz, 2H), 3.49 (t, J=6.4 Hz, 2H), 1.69-1.56 (m, 4H), 1.42-1.40 (m, 4H).

Example 110. Synthesis of 6-(benzyloxy)hexanal

This example illustrates synthesis of:

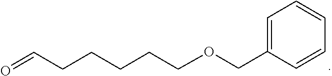 [SMILES: O=CCCCCCOCc1ccccc1]

To a stirred solution of 6-(benzyloxy)hexan-1-ol (11 g, 52.8 mmol, 1.0 eq.) in dichloromethane (150 mL), added pyridinium chlorochromate (15.5 g, 79.2 mmol, 1.5 eq.) at 0° C. and the reaction mixture was stirred at room temperature for 2 h. The progress of reaction was monitored by TLC (starting materials were consumed). The reaction mixture was diluted with pentane (500 mL) and stirred for 30 minutes. Then, reaction mixture was filtered through celite bed and wash with pentane (3×200 mL). The organic layer was collected and dried over sodium sulphate, filtered and concentrated under reduce pressure. The crude was purified by flash column chromatography (silica gel, 0-25% gradient of ethyl acetate in hexane) to get 6-(benzyloxy)hexanal (9.4 g, Yield=86.29%) as a colorless liquid. ¹H-NMR (400 MHz, CDCl₃-d3): δ 9.76 (t, J=1.6 Hz, 1H), 7.36-7.27 (m, 5H), 4.49 (s, 2H), 3.47 (t, J=6.4 Hz, 2H), 2.45-2.41 (m, 2H), 1.69-1.60 (m, 4H), 1.49-1.33 (m, 2H).
Example 111. Synthesis of {[(7-methyloct-6-en-1-yl)oxy]methyl}benzene

This example illustrates synthesis of:

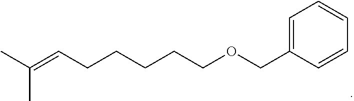 [SMILES: CC(C)=CCCCCCOCc1ccccc1]

To a stirred solution of triphenyl(propan-2-yl)phosphanium bromide (9.86 g, 25.6 mmol, 1.2 eq.) in tetrahydrofuran (100 mL), was added lithium(1+) butan-1-ide (2.05 g, 32 mmol, 1.5 eq.) dropwise at −78° C. The reaction mixture was stirred at room temperature for 1 h. Then, the mixture was cooled again to −78° C. and 6-(benzyloxy)hexanal (4.4 g, 21.3 mmol) (dissolved in THF) was added dropwise. The reaction mixture was stirred at room temperature for 16 h. The progress of reaction was monitored by TLC (starting materials were consumed). The reaction mixture was quenched with aq. ammonium chloride solution and extracted with ethyl acetate (3×100 mL) times. The organic layer was collected and dried over sodium sulphate, filtered and concentrated under vacuum. The crude product thus obtained was purified by flash column chromatography (silica gel, 0-3% gradient of ethyl acetate in hexane) to get {[(7-methyloct-6-en-1-yl)oxy]methyl}benzene 10 (4 g, Yield=80.71%) as a colorless liquid. ¹H-NMR (400 MHz, CDCl₃-d3): δ 7.38-7.26 (m, 5H), 5.14 (t, J=7.2 Hz, 1H), 4.53 (m, 2H), 3.50 (t, J=6.8 Hz, 2H), 2.03-2.00 (m, 2H), 1.69-1.60 (m, 6H), 1.36-1.29 (m, 6H).
Example 112. Synthesis of 7-methyloctan-1-ol

This example illustrates synthesis of:

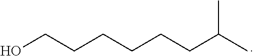

{[(7-methyloct-6-en-1-yl)oxy]methyl}benzene (8 g, 34.1 mmol, 1.0 eq.) dissolved in a mixture of methanol and tetrahydrofuran (90 mL, 1:1, v/v) was degassed with nitrogen for 5-10 minutes. Then, palladium on carbon (Pd/C, 10%)(8.02 g, w/w) was added and stirred at room temperature for 16 h under hydrogen atmosphere. The progress of reaction was monitored by TLC (starting materials were consumed). The reaction mixture was filtered through celite bed and washed with mixture (1:1, v/v) of MeOH and THF (3×200 mL). The filtrate was concentrated and purified by flash column chromatography (silica gel, 0-10% gradient of ethyl acetate in hexane) to get 7-methyloctan-1-ol (4.0 g. Yield=81.24%) as colorless liquid. ¹H NMR (400 MHz, CDCl₃-d3): δ 3.65-3.62 (m, 2H), 1.53-1.49 (m, 2H), 1.34-1.22 (m, 7H), 1.16-1.15 (m, 2H), 0.85 (d, J=6.4 Hz, 6H).
Example 113. Synthesis of 7-methyloctyl 6-bromohexanoate

This example illustrates synthesis of:

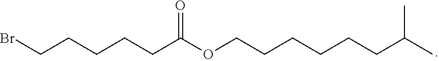 [SMILES: CC(C)CCCCCCOC(=O)CCCCCBr]

To a stirred solution of 6-bromohexanoic acid (2.44 g, 12.5 mmol, 1.0 eq.) in dichloromethane (150 mL), was added N,N-dimethyl-4-pyridylamine (1.53 g, 12.5 mmol, 1.0 eq.) and ({[3-(dimethylamino)propyl]imino}methylidene)(ethyl)amine hydrochloride (4.8 g, 25 mmol, 2.0 eq.) and stirred for 15 minutes. Then, 1-nonanol (1.8 g, 12.5 mmol, 1.0 eq.) was added and reaction mixture was stirred at room temperature for 48 h. The progress of reaction was monitored by TLC. Water (500 mL) was added to the reaction mixture and extracted with DCM (3×500 mL). The resulting organic layer was dried over sodium sulfate, and concentrated under reduce pressure, and the crude was purified by flash column chromatography (silica gel, 0-10% gradient of ethyl acetate in hexane) to get 7-methyloctyl 6-bromohexanoate (0.9 g, Yield=22.39%) as a colourless liquid. ¹H-NMR (400 MHz, CDCl₃-d3): δ 4.06 (t, J=6.8 Hz, 2H), 3.53-3.39 (m, 2H), 2.31 (t, J=7.6 Hz, 2H), 1.89-1.61 (m, 2H), 1.59-1.49 (m, 4H), 1.48-1.45 (m, 3H), 1.33-1.26 (m, 8H), 1.17-1.14 (m, 2H), 0.87 (t, J=6.8 Hz, 6H).
Example 114. Synthesis of 1-(7-fluoroheptyl)nonyl 8-(2-hydroxyethylamino)octanoate

This example illustrates synthesis of:

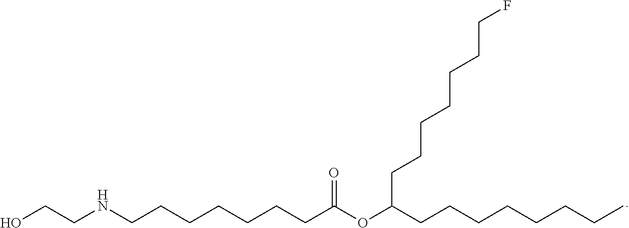 [SMILES: CCCCCCCCC(CCCCCCCF)OC(=O)CCCCCCCNCCO]

To a stirred solution of 1-(7-fluoroheptyl)nonyl 8-bromooctanoate (7.62 g, 16.4 mmol) in acetonitrile (20 mL), was added N-ethylbis(isopropyl)amine (8.55 μL, 3 eq., 49.1 mmol) and 2-aminoethanol (1 g, 16.4 mmol). Reaction mixture was stirred at 55° C. for 16 h. The progress of reaction was monitored by TLC. ELSD of reaction mixture showed 64% of desired product mass. The reaction mixture was concentrated and the crude was purified by flash column chromatography (silica gel, 0-7% gradient of methanol in DCM) to get 1-(7-fluoroheptyl)nonyl 8-(2-hydroxyethylamino)octanoate (1.2 g, Yield=16.46%) as a colorless liquid. ¹H-NMR (400 MHz, CDCl₃-d3): δ 4.85 (quin, J=6.4 Hz, 1H), 4.49 (t, J=6.4 Hz, 1H), 4.36 (t, J=6.0 Hz, 1H), 4.05-4.00 (m, 2H), 3.19-3.16 (m, 2H), 3.04-3.00 (m, 2H), 2.27 (t, J=7.6 Hz, 2H), 1.95-1.83 (m, 2H), 1.75-1.56 (m, 4H), 1.55-1.45 (m, 4H), 1.41-1.15 (m, 28H), 0.89-0.86 (m, 3H).
Example 115. Synthesis of 1-fluorohexadecan-8-yl 8-((2-hydroxyethyl)(6-((7-methyloctyl)oxy)-6-oxohexyl)amino)octanoate

This example illustrates synthesis of:

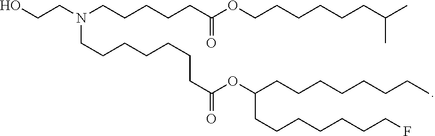 [SMILES: CCCCCCCCC(CCCCCCCF)OC(=O)CCCCCCCN(CCO)CCCCCC(=O)OCCCCCCC(C)C]

To a stirred solution of 1-(7-fluoroheptyl)nonyl 8-(2-hydroxyethylamino)octanoate (624 mg, 1.4 mmol, 1.0 eq.) in acetonitrile (10 mL), was added N-ethylbis(isopropyl)amine (0.732 μL, 4.2 mmol, 3 eq.) and 7-methyloctyl 6-bromohexanoate (450 mg, 1.4 mmol, 1.0 eq.). The reaction was stirred at 95° C. for 16 h. The progress of reaction was monitored by TLC and ELSD. The reaction mixture was concentrated under reduced pressure and the crude was purified by flash column chromatography (silica gel, 0-8% gradient of methanol in DCM) to get 1-(7-fluoroheptyl)nonyl 8-{(2-hydroxyethyl)[5-(7-methyloctyloxycarbonyl)pentyl]amino}octanoate (160 mg) as a colorless liquid. The compound (160 mg) was obtained after column chromatography was dissolved in dichloromethane (10 mL) and added anhydrous potassium carbonate (5 eq.) under nitrogen atmosphere and stirred vigorously for 2 h. Then, reaction mixture was filtered through hydrophobic PTFE syringe filter (25 mm×0.45 μm) using glass syringe to get 1-(7-fluoroheptyl)nonyl 8-{(2-hydroxyethyl)[5-(7-methyloctyloxycarbonyl)pentyl]amino}octanoate (140 mg, Yield=14.57%) as a colorless liquid. ¹H-NMR (400 MHz, CDCl₃-d3): δ 4.86 (quin, J=6.4 Hz, 1H), 4.48 (t, J=6.4 Hz, 1H), 4.36 (t, J=6.0 Hz, 1H), 4.05 (t, J=6.8 Hz, 2H), 3.54-3.50 (m, 2H), 2.56 (t, J=5.2 Hz, 2H), 2.45-2.40 (m, 4H), 2.31-2.25 (q, J=7.8 Hz, 4H), 1.72-1.67 (m, 2H), 1.66-1.57 (m, 7H), 1.54-1.40 (m, 10H), 1.34-1.25 (m, 30H), 1.17-1.14 (m, 2H), 0.89-0.86 (m, 9H). ¹⁹F-NMR (375 MHz, CDCL₃-d3): δ −218.04 ELSD analysis: Purity 99.71%, Calculated C₄₁H₈₁FNO₅, [M+H]=686.61, Observed=686.75 (m/z, M+H⁺).
Example 116. Synthesis of 9-hydroxynonyl acetate

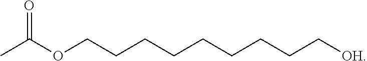

This example illustrates synthesis of: To a stirred solution of 1,9-nonanediol (150 g, 936 mmol) in tetrahydrofuran (1.24 L), pyridine (151 mL, 2 eq., 1.87 mol) and acetyl acetate (91.9 mL, 936 mmol) was added at 0° C. and stirred at 25° C. for 16 h. The reaction mixture was quenched with aqueous sodium bicarbonate solution (800 mL) and extracted with ethyl acetate (5×500 mL). The organic layer was dried over sodium sulphate, filtered and concentrated under reduced pressure. The crude was purified by flash column chromatography (silica gel, 0-50% gradient of ethyl acetate in hexane) to yield 9-hydroxynonyl acetate (80 g. Yield=42.25%) as a colorless liquid. ¹H NMR (400 MHz, CDCl₃-d3): δ 4.04 (t, J=6.8 Hz, 2H), 3.63 (t, J=6.8 Hz, 2H), 2.04 (s, 3H), 1.65-1.51 (m, 4H), 1.41-1.25 (m, 10H).

Example 117. Synthesis of 9-fluorononyl acetate

This example illustrates synthesis of:

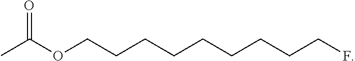

Compound 9-hydroxynonyl acetate (25 g, 124 mmol) in dichloromethane (250 mL) was cooled to −78° C. and N,N-diethyl(trifluorothio)amine (24.9 mL, 1.5 eq., 185 mmol) was added dropwise. Reaction mixtures was stirred at 25° C. for 16 h. Then reaction mass cooled to −78° C. and added dropwise aqueous sodium bicarbonate solution (35 mL) and extracted with ethyl acetate (3×100 mL). The organic layer was collected dried over sodium sulphate, filtered and evaporated under reduced pressure. The crude was purified by flash column chromatography (silica gel, 0-30% gradient of ethyl acetate in hexane) to obtain 9-fluorononyl acetate (23 g, Yield=91.1%) as colorless liquid. ¹H NMR (400 MHz, CDCl₃-d3): δ 4.49 (t, J=6.0 Hz, 1H), 3.37 (t, J=6.0 Hz, 1H), 4.05 (t, J=6.8 Hz, 2H), 2.04 (s, 3H), 1.78-1.58 (m, 4H), 1.45-1.28 (m, 10H).
Example 118. Synthesis of 9-fluoro-1-nonanol

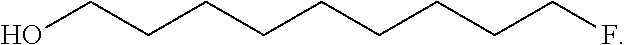 [SMILES: OCCCCCCCCCF]

This example illustrates synthesis of: 9-fluorononyl acetate (20 g, 97.9 mmol) in tetrahydrofuran (124 mL) was cooled to −78° C. and aluminium(3+) lithium tetrahydride (7.43 g, 2 eq., 196 mmol) was added dropwise. After complete addition, reaction mixture was stirred at 25° C. for 16 h. TLC showed formation of new spots and starting material was consumed. The reaction mass was cooled to −10° C. and quenched with aqueous ammonium chloride (100 mL). Then, reaction mass was filtered through celite bed. The filtrate was collected and evaporated to dryness. The crude was purified by flash column chromatography (silica gel, 0-12% gradient of ethyl acetate in hexane) to obtain 9-fluoro-1-nonanol (15 g, Yield=94.43%) as a colorless liquid. ¹H NMR (400 MHz, CDCl₃-d3): δ 4.49 (t, J=6.0 Hz, 1H), 4.37 (t, J=6.0 Hz, 1H), 3.63 (t, J=6.8 Hz, 2H), 1.75-1.61 (m, 2H), 1.58-1.51 (m, 2H), 1.45-1.28 (m, 10H).

Example 119. Synthesis of 9-fluorononanoic acid

This example illustrates synthesis of:

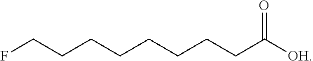 [SMILES: O=C(O)CCCCCCCCF]

A mixture of chromamethanetrione—sulfuric acid (1/1) (32.9 mL, 5 eq., 216 mmol) and water (14 mL, 777 mmol) was cooled to −10° C. Then, 9-fluoro-1-nonanol (7 g, 43.1 mmol) dissolved in acetone (70 mL) was added dropwise. The reaction mass was stirred at room temperature for 15 h. After complete consumption of starting materials (monitored by TLC), reaction mixture was dilute with water (100 mL) and extracted with diethyl ether (5×100 mL). The resulting organic layer was dried over sodium sulfate and concentrated under reduce pressure. The crude was purified by flash column chromatography (silica gel, 0-25% gradient of ethyl acetate in hexane) to obtain 9-fluorononanoic acid (4.2 g, Yield=55.24%) as a white crystal. ¹H NMR (400 MHz, CDCl₃-d3): δ 11.50-10.01 (bs, 1H), 4.49 (t, J=6.4 Hz, 1H), 4.37 (t, J=6.4 Hz, 1H), 2.35 (t, J=7.2 Hz, 2H), 1.75-1.61 (m, 4H), 1.45-1.28 (m, 8H).
Example 120. Synthesis of 2-(7-fluoroheptyl)decanoic acid

This example illustrates synthesis of:

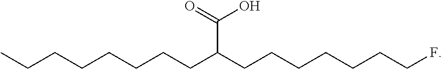 [SMILES: CCCCCCCCC(CCCCCCCF)C(=O)O]

To a stirred solution of 9-fluorononanoic acid (2.5 g, 12.7 mmol) in tetrahydrofuran (30.3 mL), was added sodium hydride (611 mg, 1.2 eq., 15.3 mmol) at 0° C. and stirred for 30 minutes. Then, lithium bis(isopropyl)azanide (1.64 g, 1.2 eq., 15.3 mmol) was added to the reaction mixture at the −50° C. The reaction mixture was stirred at room temperature for 30 minutes. Then, 1-iodooctane (3.06 g. 12.7 mmol) was added and reaction mixture was stirred at 45° C. for 16 h. The progress of reaction was monitored by TLC. The reaction mixture was poured into 1M HCl (250 mL) and extracted with ethyl acetate (3×250 mL). The resulting organic layer was dried over sodium sulfate, and concentrated under reduce pressure. The crude thus obtained was purified by flash column chromatography (silica gel, 0-2% gradient of ethyl acetate in hexane) to obtain 2-(7-fluoroheptyl)decanoic acid (0.8 g, Yield=27.77%) as a yellow liquid. ¹H NMR (400 MHz, CDCl₃-d3): δ 4.49 (t, J=6.4 Hz, 1H), 4.37 (t, J=6.4 Hz, 1H), 2.41-2.28 (m, 1H), 1.75-1.52 (m, 4H), 1.51-1.40 (m, 2H), 1.37-1.21 (m, 20H), 0.89-2.35 (m, 3H).
Example 121. Synthesis of 7-bromoheptyl 2-(7-fluoroheptyl) decanoate

This example illustrates synthesis of:

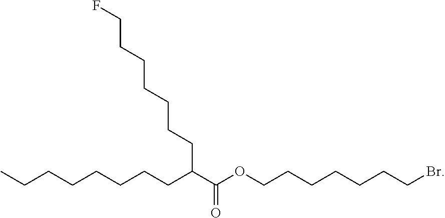 [SMILES: CCCCCCCCC(CCCCCCCF)C(=O)OCCCCCCCBr]

To a stirred solution of 2-(7-fluoroheptyl)decanoic acid (596 mg, 2.07 mmol) in dichloromethane (20 mL), was added 2-methyl-2,6,8-triaza-6,7-decadiene—hydrogen chloride (1/1) (1.19 g, 3 eq., 6.2 mmol) and N,N-dimethyl-4-pyridylamine (252 mg, 2.07 mmol). Reaction mixture was stirred for 10 minutes then 7-bromo-1-heptanol (403 mg, 2.07 mmol) was added. The reaction mixture was stirred at room temperature for 16 h. TLC showed formation of new spots and starting material was consumed. Reaction mixture was quenched with brine solution (15 mL) and extracted with dichloromethane (2×20 mL). The organic layer was dried over sodium sulphate, filtered and concentrated under reduced pressure. The crude was purified by flash column chromatography (silica gel, 0-2% gradient of ethyl acetate in hexane) to obtain 7-bromoheptyl 2-(7-fluoroheptyl) decanoate (560 mg, Yield=58.22%) as a colorless liquid. ¹H NMR (400 MHz, CDCl₃-d3): δ 4.51 (t, J=6.0 Hz, 1H), 4.39 (t, J=6.4 Hz, 1H), 4.09 (t, J=6.4 Hz, 2H), 3.43 (t, J=6.4 Hz, 2H), 2.36-2.31 (m, 1H), 1.92-1.82 (quin, J=7.2 Hz, 2H), 1.78-1.58 (m, 4H), 1.51-1.20 (m, 30H), 0.90 (t, J=6.8 Hz, 3H).
Example 122. Synthesis of 7-bromoheptyl decanoate

This example illustrates synthesis of:

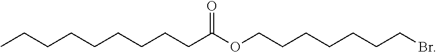 [SMILES: CCCCCCCCCC(=O)OCCCCCCCBr]

To a stirred solution of decanoic acid (5 g, 29 mmol) in dichloromethane (50 mL), 2-methyl-2,6,8-triaza-6,7-decadiene—hydrogen chloride (1/1)(16.7 g, 3 eq., 87.1 mmol) and N,N-dimethyl-4-pyridylamine (3.55 g, 29 mmol) was added and stirred for 15 minutes. Then, 7-bromo-1-heptanol (5.66 g, 29 mmol) was added and reaction mixture was stirred at room temperature for 16 h. TLC showed starting materials were consumed and formed new spots. The reaction mixture was quenched with brine solution (200 mL) and extracted with dichloromethane (3×100 mL). The organic layer was dried over sodium sulphate, filtered and concentrated under vacuum pressure. The crude product was purified by flash column chromatography (silica gel, 0-2% gradient of ethyl acetate in hexane) to get 7-bromoheptyl decanoate (7.9 g, Yield=77.91%) as colorless liquid. ¹H-NMR (400 MHz, CDCl₃-d3): δ 4.06 (t, J=6.4 Hz, 2H), 3.53 (t, J=6.8 Hz, 1H), 2.40 (t, J=6.8 Hz, 1H), 2.28 (t, J=7.6 Hz, 2H), 1.83 (quin, J=8.0 Hz, 1H), 1.77 (quin, J=8.0 Hz, 1H), 1.65-1.56 (m, 4H), 1.48-1.39 (m, 2H), 1.35-1.21 (m, 16H), 0.87 (t, J=6.8 Hz, 3H).
Example 123. Synthesis of 7-(2-hydroxyethylamino)heptyl decanoate

This example illustrates synthesis of:

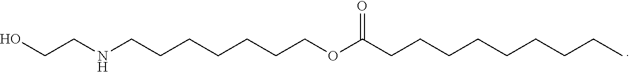 [SMILES: CCCCCCCCCC(=O)OCCCCCCCNCCO]

To a stirred solution of 7-bromoheptyl decanoate (3 g, 8.59 mmol)) in acetonitrile (20 mL), was added N-ethylbis(isopropyl)amine (3.75 mL, 2.5 eq., 21.5 mmol) and 2-aminoethanol (525 mg, 8.59 mmol). Reaction mixture was stirred at 55° C. for 16 h. After complete consumption of starting materials, reaction mass concentrated. The crude thus obtained was purified by flash column chromatography (silica gel, 0-10% gradient of methanol in DCM) to obtain 7-(2-hydroxyethylamino)heptyl decanoate (0.6 g, Yield=21.21%) as a colorless liquid. ELSD analysis: Purity 99.81%, Calculated: C₁₉H₄₀NO₃, [M+H]=330.30, Observed=330.25 (m/z, M+H⁺).
Example 124. Synthesis of 7-((7-(decanoyloxy)heptyl)(2-hydroxyethyl)amino)heptyl 2-(7-fluoroheptyl) decanoate

This example illustrates synthesis of:

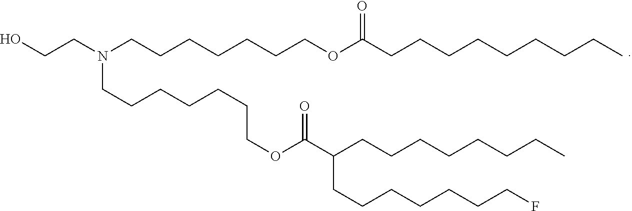 [SMILES: CCCCCCCCCC(=O)OCCCCCCCN(CCO)CCCCCCCOC(=O)C(CCCCCCCC)CCCCCCCF]

To a stirred solution of 7-(2-hydroxyethylamino)heptyl decanoate (360 mg, 1.09 mmol) in acetonitrile (10 mL), was added N-ethylbis(isopropyl)amine (572 μL, 3 eq., 3.28 mmol) and 7-bromoheptyl 2-(7-fluoroheptyl) decanoate (458 mg, 0.9 eq., 983 μmol). The reaction was stirred at 95° C. for 16 h. The progress of reaction was monitored by TLC and ELSD. The reaction mixture was concentrated under reduced pressure and the crude was purified by flash column chromatography (silica gel, 0-8% gradient of methanol in dichloromethane) to obtain 7-{(2-hydroxyethyl)[7-(nonylcarbonyloxy)heptyl]amino}heptyl 2-(7-fluoroheptyl) decanoate (135 mg) as a colorless liquid. The compound (135 mg) was obtained after column chromatography was dissolved in dichloromethane (10 mL) and added anhydrous potassium carbonate (5 eq.) under nitrogen atmosphere and stirred vigorously for 2 h. Then, reaction mixture was filtered through hydrophobic PTFE syringe filter (25 mm×0.45 μm) using glass syringe to get 7-{(2-hydroxyethyl)[7-(nonylcarbonyloxy)heptyl]amino}heptyl 2-(7-fluoroheptyl) decanoate (121 mg, Yield=15.51%) as a colorless liquid. ¹H-NMR (400 MHz, CDCl₃-d3): δ 4.48 (t, J=6.4 Hz, 1H), 4.37 (t, J=6.0 Hz, 1H), 4.09-4.01 (m, 4H), 3.51-3.48 (m, 2H), 2.56 (t, J=5.2 Hz, 2H), 2.47-2.39 (m 4H), 2.33-2.24 (m, 3H), 1.72-1.53 (m, 13H), 1.48-1.38 (m, 6H), 1.39-1.21 (m, 42H), 0.87 (t, J=6.0 Hz, 6H). ¹⁹F-NMR (375 MHz, CDCL₃-d3): δ −218.03 ELSD analysis: Purity 98.51%, Calculated C₄₃H₈₅FNO₅, [M+H]=714.64, Observed=714.80 (m/z, M+H⁺).
Example 125. Synthesis of 9-methyldecyl 6-(2-hydroxyethylamino)hexanoate

This example illustrates synthesis of:

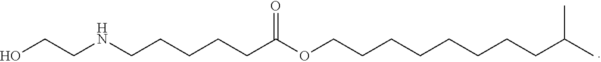 [SMILES: CC(C)CCCCCCCCOC(=O)CCCCCNCCO]

To a stirred solution of 9-methyldecyl 6-bromohexanoate (3.3 g, 9.45 mmol, 1.0 eq.) in acetonitrile (25 mL), was added N-ethylbis(isopropyl)amine (4.12 mL, 23.6 mmol, 2.5 eq.) and 2-amino-1-ethanol (577 mg, 9.45 mmol, 1.0 eq.). Reaction mixture was stirred at 55° C. for 16 h. The progress of reaction was monitored by TLC. ELSD of reaction mixture showed 74% of desired product mass. The reaction mixture was concentrated under reduced pressure. The crude thus obtained was purified by flash column chromatography (silica gel, 0-7% gradient of methanol in dichloromethane) to obtain 9-methyldecyl 6-(2-hydroxyethylamino)hexanoate (750 mg, Yield=24.09%) as a colorless liquid. ¹H NMR (400 MHz, CDCL₃-d3): δ 4.05 (t, J=6.4 Hz, 4H), 3.23-3.15 (bs, 2H), 3.10-3.02 (bs, 2H), 2.33 (t, J=7.2 Hz, 2H), 2.01-1.89 (m, 3H), 1.71-1.42 (m, 8H), 1.39-1.21 (m, 10H), 1.18-1.10 (m, 2H), 0.86 (d, J=6.4 Hz, 6H).
Example 126. Synthesis of 1-fluorohexadecan-8-yl 8-((2-hydroxyethyl)(6-((9-methyldecyl)oxy)-6-oxohexyl)amino)octanoate

This example illustrates synthesis of:

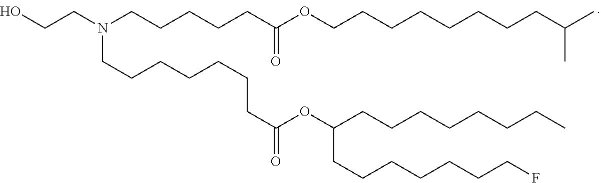 [SMILES: CCCCCCCCC(CCCCCCCF)OC(=O)CCCCCCCN(CCO)CCCCCC(=O)OCCCCCCCCC(C)C]

To a stirred solution of 9-methyldecyl 6-(2-hydroxyethylamino)hexanoate (729 mg, 2.21 mmol) in acetonitrile (10 mL), was added N-ethylbis(isopropyl)amine (966 μL, 2.5 eq., 5.53 mmol) and 1-(7-fluoroheptyl)nonyl 8-bromooctanoate (1.03 g, 2.21 mmol). The reaction was stirred at 95° C. for 16 h. The progress of reaction was monitored by TLC and ELSD. The reaction mixture was concentrated under reduced pressure and the crude was purified by flash column chromatography (silica gel, 0-8% gradient of methanol in dichloromethane) to obtain 1-(7-fluoroheptyl)nonyl 8-{(2-hydroxyethyl)[5-(9-methyldecyloxycarbonyl)pentyl]amino}octanoate (135 mg) as a colorless liquid. The compound (135 mg) was obtained after column chromatography was dissolved in dichloromethane (10 mL) and added anhydrous potassium carbonate (5 eq.) under nitrogen atmosphere and stirred vigorously for 2 h. Then, reaction mixture was filtered through hydrophobic PTFE syringe filter (25 mm×0.45 μm) using glass syringe to obtain 1-(7-fluoroheptyl)nonyl 8-{(2-hydroxyethyl)[5-(9-methyldecyloxycarbonyl)pentyl]amino}octanoate (120 mg, Yield=7.06%) as a colorless liquid. ¹H NMR (400 MHz, CDCL₃-d3): δ 4.86 (quin, J=6.4 Hz, 1H), 4.48 (t, J=6.0 Hz, 1H), 4.37 (t, J=6.0 Hz, 1H), 4.05 (t, J=6.4 Hz, 2H), 3.51 (t, J=5.6 Hz, 2H), 2.55 (t, J=5.6 Hz, 2H), 2.47-2.39 (m, 4H), 2.28 (q, J=8.0 Hz, 4H), 1.75-1.55 (m, 9H), 1.54-1.37 (m, 12H), 1.35-1.21 (m, 34H), 1.19-1.10 (m, 2H), 0.89-0.82 (m, 9H). ELSD analysis: Purity 98.18%, Calculated for C₄₃H₈₅FNO₅, [M+H]=714.64, Observed=714.80 (m/z, M+H⁺).
Example 127. Synthesis of 1-octylnonyl 6-(2-hydroxyethylamino)hexanoate

This example illustrates synthesis of:

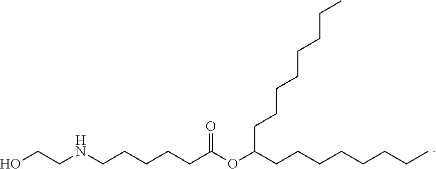 [SMILES: CCCCCCCCC(CCCCCCCC)OC(=O)CCCCCNCCO]

To a stirred solution of 1-octylnonyl 6-bromohexanoate (4.6 g, 10.6 mmol, 1 eq.) in acetonitrile (40 mL), was added N-ethylbis(isopropyl)amine (5.52 mL, 31.8 mmol, 3 eq.) and 2-aminoethanol (648 mg, 10.6 mmol) and reaction mixture was stirred at 55° C. for 16 h. TLC showed starting materials were consumed and formed new spots. The reaction mixture was concentrated and crude thus obtained was purified by flash column chromatography (silica gel, 0-7% methanol in dichloromethane) to get 1-octylnonyl 6-(2-hydroxyethylamino)hexanoate (0.9 g, Yield=20.5%) as a colorless liquid. ¹H-NMR (400 MHz, CDCl₃-d₃): δ 4.86 (quin, J=6.4 Hz, 1H), 3.69 (t, J=5.2 Hz, 2H), 2.83 (t, J=5.2 Hz, 2H), 2.68 (t, J=7.2 Hz, 2H), 2.31 (t, J=7.2 Hz, 2H), 1.68-1.53 (m, 4H), 1.50-1.49 (m, 4H), 1.41-1.37 (m, 2H), 1.35-1.25 (m, 24H), 3.69 (t, J=6.8 Hz, 6H). ELSD analysis: Purity 98.46%, Calculated: C₂₅H₅₂NO₃, [M+H]=414.30, Observed=414.40 (m/z, M+H⁺).
Example 128. Synthesis of 1-fluorohexadecan-8-yl 8-((6-(heptadecan-9-yloxy)-6-oxohexyl)(2-hydroxyethyl)amino)octanoate

This example illustrates synthesis of:

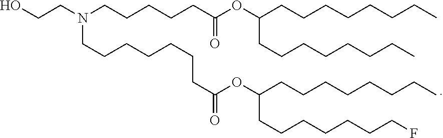 [SMILES: CCCCCCCCC(CCCCCCCF)OC(=O)CCCCCCCN(CCO)CCCCCC(=O)OC(CCCCCCCC)CCCCCCCC]

To a stirred solution of 1-octylnonyl 6-(2-hydroxyethylamino)hexanoate (0.5 g, 1.21 mmol) in acetonitrile (10 mL), was added N-ethylbis(isopropyl)amine (0.632 μL, 3 eq., 3.63 mmol) and 1-(7-fluoroheptyl)nonyl 8-bromooctanoate (563 mg, 1.21 mmol). The reaction was stirred at 95° C. for 16 h. The progress of reaction was monitored by TLC and ELSD. The reaction mixture was concentrated under reduced pressure and the crude thus obtained was purified by flash column chromatography (silica gel, 0-8% gradient of methanol in dichloromethane) to get 1-(7-fluoroheptyl)nonyl 8-{(2-hydroxyethyl)[5-(1-octylnonyloxycarbonyl)pentyl]amino}octanoate (190 mg) as a pale yellow liquid. The compound (190 mg) was obtained after column chromatography was dissolved in dichloromethane (10 mL) and added anhydrous potassium carbonate (5 eq.) under nitrogen atmosphere and stirred vigorously for 2 h. Then, reaction mixture was filtered through hydrophobic PTFE syringe filter (25 mm×0.45 μm) using glass syringe to get 1-(7-fluoroheptyl)nonyl 8-{(2-hydroxyethyl)[5-(1-octylnonyloxycarbonyl)pentyl]amino}octanoate (170 mg, Yield=17.62%) as a pale yellow liquid. ¹H-NMR (400 MHz, CDCl₃-d3): δ 4.86 (quin, J=6.4 Hz, 2H), 4.48 (t, J=6.0 Hz, 1H), 4.36 (t, J=6.0 Hz, 1H), 3.52 (t, J=5.2 Hz, 2H), 2.57 (t, J=4.8 Hz, 2H), 2.45-2.44 (m, 4H), 2.30-2.25 (m, 4H), 1.72-1.59 (m, 5H), 1.50-1.34 (m, 14H), 1.29-1.25 (m, 52H), 0.87 (t, J=6.4 Hz, 9H). ¹⁹F-NMR (375 MHz, CDCL₃-d3): δ −218.03 ELSD analysis: Purity 99.16%, Calculated C₄₉H₉₇FNO₅, [M+H]=798.74, Observed=798.55 (m/z, M+H⁺).

### Example 129. Synthesis of 1-octylnonyl 6-(3-hydroxypropylamino)hexanoate

This example illustrates synthesis of:

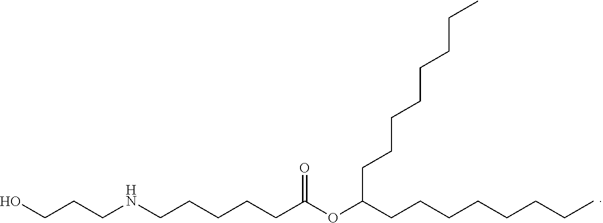 [SMILES: CCCCCCCCC(CCCCCCCC)OC(=O)CCCCCNCCCO]

To a stirred solution of nonyl 8-bromooctanoate (3 g, 8.59 mmol) in acetonitrile (20 mL), was added N-ethylbis(isopropyl)amine (4.5 mL, 3 eq., 25.8 mmol) and 3-amino-1-propanol (645 mg, 8.59 mmol). Reaction mixture was stirred at 55° C. for 16 h. After complete consumption of starting materials (monitored by TLC), reaction mass concentrated. The crude thus obtained was purified by flash column chromatography (silica gel, 0-10% gradient of methanol in DCM) to obtain 1-octylnonyl 6-(3-hydroxypropylamino)hexanoate (0.5 g, Yield=16.95%) as a colorless liquid. ¹H-NMR (400 MHz, CDCl₃-d3): δ 4.04 (t, J=6.8 Hz, 2H), 3.84 (t, J=5.2 Hz, 2H), 3.02 (t, J=5.6 Hz, 2H), 2.76 (t, J=7.6 Hz, 2H), 2.28 (t, J=7.6 Hz, 2H), 1.88-1.85 (m, 2H), 1.62-1.61 (m, 6H), 1.41-1.26 (m, 19H), 0.89-0.86 (m, 3H).
Example 130. Synthesis of 1-fluoroheptadecan-9-yl 8-((3-hydroxypropyl)(8-(nonyloxy)-8-oxooctyl)amino)octanoate

This example illustrates synthesis of:

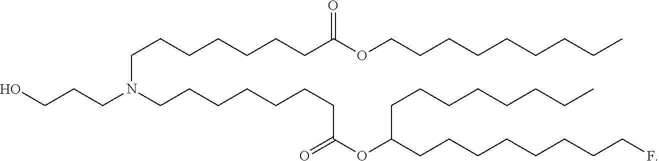 [SMILES: CCCCCCCCCOC(=O)CCCCCCCN(CCCO)CCCCCCCC(=O)OC(CCCCCCCC)CCCCCCCCF]

To a stirred solution of 9-fluoro-1-octylnonyl 8-bromooctanoate (628 mg, 1.31 mmol) in acetonitrile (5 mL), was added N-ethylbis(isopropyl)amine (0.7 mL, 3 eq., 3.93 mmol) and nonyl 8-(3-hydroxypropylamino)octanoate (450 mg, 1.31 mmol). The reaction was stirred at 90° C. for 16 h. The progress of reaction was monitored by TLC and ELSD. After the starting materials were completely consumed, reaction mixture was concentrated under reduced pressure. The crude was purified by flash column chromatography (silica gel, 0-8% gradient of methanol in DCM) to get 9-fluoro-1-octylnonyl 8-{(3-hydroxypropyl)[7-(nonyloxycarbonyl)heptyl]amino}octanoate (137 mg) as pale-yellow liquid. The compound (137 mg) obtained after column chromatography was dissolved in dichloromethane (10 mL), was added 5 equivalent of anhydrous potassium carbonate under nitrogen atmosphere and stirred reaction mixture vigorously 2 h then filtered through hydrophobic PTFE syringe filter (25 mm×0.45 μm) using glass syringe to get 9-fluoro-1-octylnonyl 8-{(3-hydroxypropyl)[7-(nonyloxycarbonyl)heptyl]amino}octanoate (122 mg, Yield=12.55%) as pale yellow liquid. ¹H-NMR (400 MHz, CDCl₃-d3): δ 4.86 (quin, J=6.0 Hz, 1H), 4.48 (t, J=6.0 Hz, 1H), 4.37 (t, J=6.0 Hz, 1H), 4.04 (t, J=6.0 Hz, 2H), 3.80 (t, J=4.8 Hz, 2H), 2.76-2.42 (bs, 4H), 2.30-2.25 (m, 4H), 1.80 (m, 1H), 1.77-1.71 (m, 2H), 1.67-1.57 (m, 7H), 1.50-1.49 (m, 4H), 1.32-1.25 (m, 52H), 0.89-0.85 (m, 6H). ¹⁹F-NMR (375 MHz, CDCL₃-d3): δ −217.986 ELSD analysis: Purity 99.14%, Calculated C₄₅H₈₉FNO₅, [M+H]=742.67, Observed=742.80 (m/z, M+H⁺).
Example 131. Synthesis of nonyl 8-(4-hydroxybutylamino)octanoate

This example illustrates synthesis of:

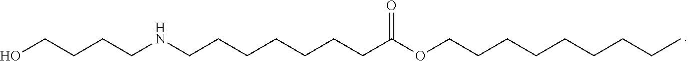 [SMILES: CCCCCCCCCOC(=O)CCCCCCCNCCCCO]

To a stirred solution of nonyl 8-bromooctanoate 10 (3.0 g, 8.59 mmol, 1.0 eq.) in acetonitrile (20 mL), was added N-ethylbis(isopropyl)amine (3.75 mL, 21.5 mmol, 2.5 eq.) and 4-amino-1-butanol (765 mg, 8.59 mmol, 1.0 eq.). Reaction mixture was stirred at 55° C. for 16 h. ELSD of reaction mixture showed 64% of desired product mass. The reaction mixture was concentrated and the crude was purified by flash column chromatography (silica gel, 0-7% gradient of methanol in dichloromethane) to obtain nonyl 8-(4-hydroxybutylamino)octanoate (0.5 g, Yield=16.28%) as a colorless liquid. ¹H-NMR (400 MHz, CDCl₃-d3): δ 4.03 (t, J=6.8 Hz, 2H), 3.72 (t, J=5.2 Hz, 2H), 3.16-2.94 (m, 6H), 2.27 (t, J=7.6 Hz, 2H), 2.06-2.00 (m, 2H), 1.88-1.83 (m, 2H), 1.76-1.70 (m, 2H), 1.60-1.55 (m, 4H), 1.63-1.26 (m, 16H), 0.87 (t, J=6.8 Hz, 3H). ELSD analysis: Purity 99.46%, Calculated: C₂₁H₄₄NO₃, [M+H]=358.33, Observed=358.40 (m/z, M+H⁺).
Example 132. Synthesis of 1-fluoroheptadecan-9-yl 8-((4-hydroxybutyl)(8-(nonyloxy)-8-oxooctyl)amino)octanoate

This example illustrates synthesis of:

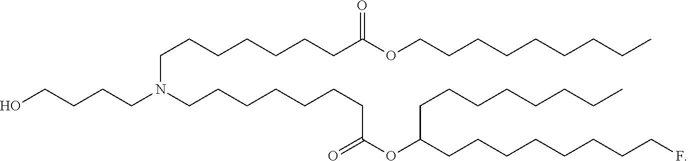 [SMILES: CCCCCCCCCOC(=O)CCCCCCCN(CCCCO)CCCCCCCC(=O)OC(CCCCCCCC)CCCCCCCCF]

To a stirred solution of nonyl 8-(4-hydroxybutylamino)octanoate (0.5 g, 1.4 mmol, 1.0 eq.) in acetonitrile (10 mL), was added N-ethylbis(isopropyl)amine (542 mg, 4.19 mmol, 3.0 eq.) and 9-fluoro-1-octylnonyl 8-bromooctanoate (671 mg, 1.4 mmol, 1.0 eq.). The reaction was stirred at 95° C. for 16 h. The progress of reaction was monitored by TLC and ELSD (37% desired product mass was observed in the reaction mixture by ELSD). The reaction mixture was concentrated under reduced pressure and the crude was purified by flash column chromatography (silica gel, 0-8% gradient of methanol in DCM) to obtain 9-fluoro-1-octylnonyl 8-{(4-hydroxybutyl)[7-(nonyloxycarbonyl)heptyl]amino}octanoate (200 mg) as a pale yellow liquid. The compound (200 mg) was obtained after column chromatography was dissolved in dichloromethane (10 mL) and added anhydrous potassium carbonate (5 eq.) under nitrogen atmosphere and stirred vigorously for 2 h. Then, reaction mixture was filtered through hydrophobic PTFE syringe filter (25 mm×0.45 μm) using glass syringe to get 9-fluoro-1-octylnonyl 8-{(4-hydroxybutyl)[7-(nonyloxycarbonyl)heptyl]amino}octanoate (180 mg, Yield=17.02%) as a pale yellow liquid. ¹H-NMR (400 MHz, CDCl₃-d3): δ 4.85 (quin, J=6.4 Hz, 1H), 4.48 (t, J=6.0 Hz, 1H), 4.37 (t, J=6.0 Hz, 1H), 4.04 (t, J=6.8 Hz, 2H), 3.53 (t, J=4.8 Hz, 2H), 2.43-2.39 (m, 6H), 2.30-2.25 (m, 4H), 1.72-1.57 (m, 10H), 1.50-1.39 (m, 8H), 1.39-1.25 (m, 48H), 0.89-0.85 (m, 6H). ¹⁹F-NMR (375 MHz, CDCL₃-d3): δ −217.98 ELSD analysis: Purity 99.82%, Calculated C₄₆H₉₁FNO₅. [M+H]=756.68, Observed=756.50 (m/z, M+H⁺).
Example 133. Synthesis of 7-bromoheptyl decanoate

This example illustrates synthesis of:

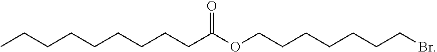 [SMILES: CCCCCCCCCC(=O)OCCCCCCCBr]

To a stirred solution of decanoic acid (5 g, 29 mmol) in dichloromethane (50 mL), 2-methyl-2,6,8-triaza-6,7-decadiene-hydrogen chloride (1/1)(16.7 g, 3 eq., 87.1 mmol) and N,N-dimethyl-4-pyridylamine (3.55 g, 29 mmol) was added and stirred for 15 minutes. Then, 7-bromo-1-heptanol (5.66 g, 29 mmol) was added and reaction mixture was stirred at room temperature for 16 h. TLC showed starting materials were consumed and formed new spots. The reaction mixture was quenched with brine solution (200 mL) and extracted with dichloromethane (3×100 mL). The organic layer was dried over sodium sulphate, filtered and concentrated under vacuum pressure. The crude product was purified by flash column chromatography (silica gel, 0-2% gradient of ethyl acetate in hexane) to get 7-bromoheptyl decanoate (7.9 g, Yield=77.91%) as colorless liquid. ¹H-NMR (400 MHz, CDCl₃-d3): δ 4.06 (t, J=6.4 Hz, 2H), 3.53 (t, J=6.8 Hz, 1H), 3.40 (t, J=6.8 Hz, 1H), 2.28 (t, J=7.6 Hz, 2H), 1.83 (quin, J=8.0 Hz, 1H), 1.77 (quin, J=8.0 Hz, 1H), 1.65-1.56 (m, 4H), 1.48-1.39 (m, 2H), 1.35-1.21 (m, 16H), 0.87 (t, J=6.8 Hz, 3H).

Example 134. Synthesis of 7-(2-hydroxyethylamino)heptyl decanoate

This example illustrates synthesis of:

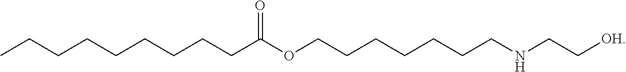 [SMILES: CCCCCCCCCC(=O)OCCCCCCCNCCO]

To a stirred solution of 7-bromoheptyl decanoate (3 g, 8.59 mmol)) in acetonitrile (20 mL), was added N-ethylbis(isopropyl)amine (3.75 mL, 2.5 eq., 21.5 mmol) and 2-aminoethanol (525 mg, 8.59 mmol). Reaction mixture was stirred at 55° C. for 16 h. After complete consumption of starting materials, reaction mass concentrated. The crude thus obtained was purified by flash column chromatography (silica gel, 0-10% gradient of methanol in DCM) to obtain 7-(2-hydroxyethylamino)heptyl decanoate (0.6 g. Yield=21.21%) as a colorless liquid. ELSD analysis: Purity 99.81%, Calculated: C₁₉H₄₀NO₃, [M+H]=330.30, Observed=330.25 (m/z, M+H⁺).
Example 135. Synthesis of 7-((7-(decanoyloxy)heptyl)(2-hydroxyethyl)amino)heptyl 10-fluoro-2-octyldecanoate

This example illustrates synthesis of:

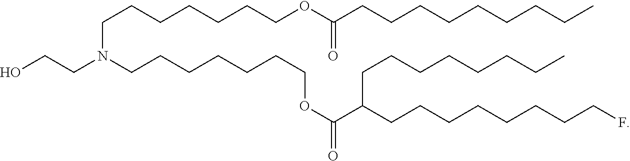 [SMILES: CCCCCCCCCC(=O)OCCCCCCCN(CCO)CCCCCCCOC(=O)C(CCCCCCCC)CCCCCCCCF]

To a stirred solution of 7-bromoheptyl 10-fluoro-2-octyldecanoate (590 mg, 1.23 mmol) in acetonitrile (10 mL), was added N-ethylbis(isopropyl)amine (537 μL, 2.5 eq., 3.08 mmol) and 7-(2-hydroxyethylamino)heptyl decanoate (405 mg, 1.23 mmol). The progress of reaction was monitored by TLC and ELSD. The reaction mixture was concentrated under reduced pressure and the crude was purified by flash column chromatography (silica gel, 0-4% gradient of methanol in dichloromethane) to obtain 7-{(2-hydroxyethyl)[7-(nonylcarbonyloxy)heptyl]amino}heptyl 10-fluoro-2-octyldecanoate (160 mg, Yield=17.89%) as a colorless liquid. ¹H-NMR (400 MHz, CDCl₃-d3): δ 4.48 (t, J=6.0 Hz, 1H), 4.37 (t, J=6.0 Hz, 1H), 4.07-4.03 (m, 4H), 3.69-3.55 (m, 2H), 2.75-2.65 (bs, 2H), 2.61-2.49 (s, 4H), 2.31-2.26 (m, 3H), 1.75-1.48 (m, 12H), 1.45-1.18 (m, 50H), 0.87 (t, J=6.4 Hz, 6H). ¹⁹F-NMR (375 MHz, CDCL₃-d3): δ −217.99 ELSD analysis: Purity 99.56%, Calculated C₄₄H₈₇FNO₅, [M+H]=728.66, Observed=728.75 (m/z, M+H⁺).
Example 136. Synthesis of 1-fluoroheptadecan-9-yl 8-((2-hydroxyethyl)(6-oxo-6-(undecyloxy)hexyl)amino)octanoate

This example illustrates synthesis of:

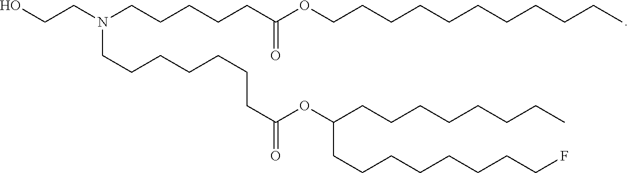 [SMILES: CCCCCCCCCCCOC(=O)CCCCCN(CCO)CCCCCCCC(=O)OC(CCCCCCCC)CCCCCCCCF]

To a stirred solution of 9-fluoro-1-octylnonyl 8-bromooctanoate (728 mg, 1.52 mmol) in acetonitrile (5.0 mL), was added N-ethylbis(isopropyl)amine (795 μL, 3 eq., 4.55 mmol) and undecyl 6-(2-hydroxyethylamino)hexanoate (0.5 g, 1.52 mmol). The reaction was stirred at 90° C. for 16 h. The progress of reaction was monitored by TLC and ELSD. After the starting materials were completely consumed, reaction mixture was concentrated under reduced pressure. The crude was purified by flash column chromatography (silica gel, 0-8% gradient of methanol in DCM) to get 9-fluoro-1-octylnonyl 8-{(2-hydroxyethyl)[5-(undecyloxycarbonyl)pentyl]amino}octanoate (0.1 g. Yield=14.54%) as pale-yellow liquid. The compound (120 mg) was obtained after column chromatography was dissolved in dichloromethane (10 mL) and added anhydrous potassium carbonate (5 eq.) under nitrogen atmosphere and stirred vigorously for 2 h. Then, reaction mixture was filtered through hydrophobic PTFE syringe filter (25 mm×0.45 μm) using glass syringe to get 9-fluoro-1-octylnonyl 8-{(2-hydroxyethyl)[5-(undecyloxycarbonyl)pentyl]amino}octanoate (0.1 g. Yield=14.54%) as pale-yellow liquid. ¹H-NMR (400 MHz, CDCl₃-d3): δ 4.86 (quin, J=6.0 Hz, 1H), 4.48 (t, J=6.0 Hz, 1H), 4.37 (t, J=6.0 Hz, 1H), 4.05 (t, J=6.0 Hz, 2H), 3.51 (t, J=5.2 Hz, 2H), 2.56 (t, J=5.6 Hz, 2H), 2.47-2.40 (m, 4H), 2.28 (q, J=7.6 Hz, 4H), 1.72-1.57 (m, 8H), 1.50-1.40 (m, 10H), 1.30-1.25 (m, 44H), 0.89-0.86 (m, 6H). ¹⁹F-NMR (375 MHz, CDCl₃-d3): δ −217.99 ELSD analysis: Purity 99.54%, Calculated C₄₄H₈₇FNO₅, [M+H]=728.66, Observed=728.75 (m/z, M+H⁺).
Example 137. Synthesis of 1-fluoroheptadecan-9-yl 8-((2-hydroxyethyl)(6-(nonyloxy)-6-oxohexyl)amino)octanoate

This example illustrates synthesis of:

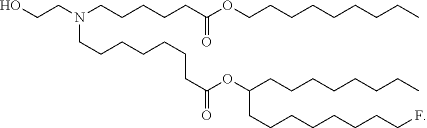 [SMILES: CCCCCCCCCOC(=O)CCCCCN(CCO)CCCCCCCC(=O)OC(CCCCCCCC)CCCCCCCCF]

To a stirred solution of nonyl 6-(2-hydroxyethylamino)hexanoate (0.5 g, 1.1 eq., 1.66 mmol) in acetonitrile (5.2 mL), was added N-ethylbis(isopropyl)amine (777 μL, 3 eq., 4.37 mmol) and 9-fluoro-1-octylnonyl 8-bromooctanoate (698 mg, 1.46 mmol). The reaction was stirred at 90° C. for 16 h. The progress of reaction was monitored by TLC and ELSD. The ELSD of reaction mixture showed 40% of desired product mass. Reaction mixture was concentrated under reduced pressure and the crude was purified by flash column chromatography (silica gel, 0-8% gradient of methanol in dichloromethane) to obtain desired product (350 mg) with LCMS purity is 97%. Further, compound obtain after column chromatography was purified by preparative HPLC to get 9-fluoro-1-octylnonyl 8-{(2-hydroxyethyl)[5-(nonyloxycarbonyl)pentyl]amino}octanoate (170 mg, Yield=16.68%) as a colorless liquid. ¹H-NMR (400 MHz, CDCl₃-d3): δ 4.86 (quin, J=6.4 Hz, 1H), 4.48 (t, J=6.4 Hz, 1H), 4.37 (t, J=6.0 Hz, 1H), 4.05 (t, J=6.8 Hz, 2H), 3.51 (t, J=5.6 Hz, 2H), 2.56 (t, J=5.6 Hz, 2H), 2.47-2.40 (m, 4H), 2.28 (q, J=7.6 Hz, 4H), 1.72-1.57 (m, 8H), 1.50-1.40 (m, 8H), 1.30-1.25 (m, 42H), 0.89-0.86 (m, 6H). ¹⁹F-NMR (375 MHz, CDCl₃-d3): δ −222.76 ELSD analysis: Purity 99.42%, Calculated C₄₂H₈₃FNO₅, [M+H]=700.63, Observed=700.75 (m/z, M+H⁺).
Example 138. Synthesis of 1-fluoroheptadecan-9-yl 8-((6-(heptadecan-9-yloxy)-6-oxohexyl)(2-hydroxyethyl)amino)octanoate

This example illustrates synthesis of:

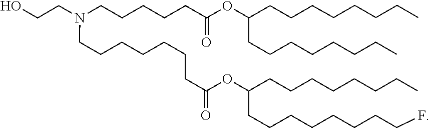 [SMILES: CCCCCCCCC(CCCCCCCCF)OC(=O)CCCCCCCN(CCO)CCCCCC(=O)OC(CCCCCCCC)CCCCCCCC]

To a stirred solution of 9-fluoro-1-octylnonyl 8-bromooctanoate (360 mg, 0.9 eq., 751 μmol) in acetonitrile (5 mL, 95.7 mmol), was added 1-octylnonyl 6-(2-hydroxyethylamino)hexanoate (0.4 g. 1.2 eq., 967 μmol) and N-ethylbis(isopropyl)amine (437 μL, 3 eq., 2.5 mmol) to it. The reaction was stirred at 100° C. for 48 h. The progress of reaction was monitored by TLC/ELSD (78% product formation in reaction mixture by ELSD). The reaction mixture was concentrated under reduced pressure and the crude was purified by flash column chromatography (SiO₂: 0-12% Methanol in Dichloromethane), to give 9-fluoro-1-octylnonyl 8-{(2-hydroxyethyl)[5-(1-octylnonyloxy carbonyl)pentyl]amino}octanoate (290 mg) with ELSD purity is 97% Further, compound obtain after column chromatography was purified by preparative HPLC to get 9-fluoro-1-octylnonyl 8-{(2-hydroxyethyl)[5-(1-octylnonyloxycarbonyl)pentyl]amino}octanoate (150 mg, yield=22.14%) as a colorless liquid. The compound was dissolved in dichloromethane (5 mL) under a nitrogen atmosphere and filtered through a hydrophobic PTFE syringe filter (25 mm×0.45 μm) using a glass syringe to give the desired compound. ¹H-NMR (400 MHz, CDCl₃-d3): δ 4.86 (quin, J=6.0 Hz, 2H), 4.48 (t, J=6.0 Hz, 1H), 4.37 (t, J=6.0 Hz, 1H), 3.51 (t, J=5.2 Hz, 2H), 2.56 (t, J=5.6 Hz, 2H), 2.45-2.40 (m, 4H), 2.30-2.25 (m, 4H), 1.72-1.59 (m, 7H), 1.50-1.39 (m, 15H), 1.30-1.25 (m, 50H), 0.87 (t, J=6.8 Hz, 6H). ¹⁹F-NMR (375 MHz, CDCl₃-d3): δ −218.002 ELSD analysis: Purity 99.26%, Calculated C₅₀H₉₉FNO₅, [M+H]=812.74, Observed=812.85 (m/z, M+H⁺).
Example 139. Synthesis of 3-(8-bromooctanoyl)-1,3-oxazolidin-2-one

This example illustrates synthesis of:

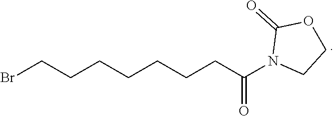 [SMILES: O=C(CCCCCCCBr)N1CCOC1=O]

To a stirred solution of 8-bromooctanoic acid (10 g, 89.6 mmol, 1.0 eq) in dichloromethane (600 ml) and dimethylformamide (2 ml), was added oxalyl dichloride (38.4 ml, 448 mmol, 5.0 eq) at 0° C. under nitrogen atmosphere and stirred at r.t. for 16 h. After completion of the reaction (monitored by TLC using MeOH to formation Methyl ester). The reaction mixture was concentrated to get 8-bromooctanoyl chloride (12.0 g, Yield=96.98%) as a yellow solid. This intermediate was used in the next step without further purification. To a stirred solution of 1,3-oxazolidin-2-one (4.33 g, 49.7 mmol, 1.0 eq) in dichloromethane (150 ml) was added N,N-dimethyl-4-pyridylamine (1.21 g, 9.94 mmol, 0.2 eq) and triethylamine (27.9 mL, 4 eq., 199 mmol) and stirred it to 15 min at 0° C. Then 8-bromooctanoyl chloride (12 g, 49.7 mmol) in dichloromethane (10 ml) was added dropwise into the reaction mixture and stirred reaction mixture overnight. The progress of reaction was monitored by TLC/ELSD (86% product form in reaction mixture by ELSD). The reaction was concentrated in vacuum which was dissolved with EtOAc (100 mL) and washed with 3% HCl (100 mL). The resulting organic layer was dried over Na₂SO₄, and concentrated under reduce pressure, and the crude was purified by flash column chromatography (SiO₂: 0-3% Ethyl acetate in Hexane) to get 3-(8-bromooctanoyl)-1,3-oxazolidin-2-one (6.7 g, Yield=46.16%) as a thick oil. ¹H NMR (400 MHz, CDCl₃-d₃): δ 4.41 (t, J=8.0 Hz, 2H), 4.01 (t, J=8.0 Hz, 2H), 3.54-3.38 (m, 2H), 2.93-2.89 (t, J=7.2 Hz, 2H), 1.88-1.73 (m, 2H), 1.68-1.62 (m, 2H), 1.47-1.35 (m, 6H). ELSD analysis: Purity 92.99%, Calculated C₁₁H₁₉BrNO₃, [M+H]=292.06, Observed=292.15 (m/z, M+H⁺).
Example 140. Synthesis of 3-[(S)-8-bromo-2-methyloctanoyl]-1,3-oxazolidin-2-one

This example illustrates synthesis of:

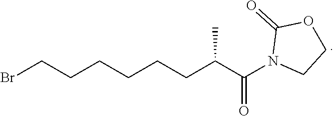 [SMILES: C[C@@H](CCCCCCBr)C(=O)N1CCOC1=O]

To a stirred solution of 3-(8-bromooctanoyl)-1,3-oxazolidin-2-one (6.7 g. 22.9 mmol, 1.0 eq) in tetrahydrofuran (135 ml) was added sodium bis(trimethylsilyl)azanide (5.47 g, 29.8 mmol, 1.3 eq) dropwise at −78° C. and the reaction mixture was stirred at same temperature for 1 hr, then iodomethane (7.14 mL, 115 mmol, 5 eq) (dissolved in THF) was added dropwise and the reaction mixture was stirred at −78° C. for 4 h. After completion of reaction (monitored by TLC), the reaction mixture was quenched with aq. NH₄Cl solution and extracted with dichloromethane (100 mL×3) times, washed consecutively with 5% KHSO₄ (100 ml), saturated Na₂S₂O₃(100 ml) and brine (100 ml). The resulting organic layer was dried over Na₂SO₄, and concentrated under reduce pressure, and the crude was purified by flash column chromatography (SiO₂: 0-3% Ethyl acetate in Hexane), to get 3-[(S)-8-bromo-2-methyloctanoyl]-1,3-oxazolidin-2-one (4.1 g, Yield=58.39%) as a colorless liquid. ¹H-NMR (400 MHz, CDCl₃-d₃): δ 4.40 (t, J=8.0 Hz, 2H), 4.02 (t, J=8.0 Hz, 2H), 3.76-3.69 (m, 1H), 3.53-3.37 (m, 2H), 1.87-1.80 (m, 2H), 1.78-1.73 (m, 2H), 1.42-1.31 (br, 6H), 1.15 (d, J=7.2 Hz, 3H).
Example 141. Synthesis of 8-bromo-2-methyloctanoic acid

This example illustrates synthesis of:

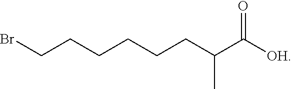

To a stirred solution of 3-[(S)-8-bromo-2-methyloctanoyl]-1,3-oxazolidin-2-one (4.1 g, 13.4 mmol, 1.0 eq) in THF (90 ml) and water (30 mL), was added hydrogen peroxide (10.5 ml, 134 mmol, 10 eq) and lithium hydroxide (962 mg. 40.2 mmol, 3 eq) to it. The reaction mixture was stirred for 3 h, after completion of reaction (monitored by TLC). The reaction mixture quenched with Na₂SO₃, acidified up to pH 2 with the help of 5% KHSO₄ and extracted with ethyl acetate (100 mL×3), The resulting organic layer was dried over Na₂SO₄, and concentrated under reduce pressure to get 8-bromo-2-methyloctanoic acid (2.8 g, 11.8 mmol) as a colorless liquid, which was used in the next step without further purification. ¹H NMR (400 MHz, CDCl₃-d3): δ 3.54-3.38 (m, 2H), 2.50-2.41 (m, 1H), 1.91-1.64 (m, 3H), 1.45-1.39 (m, 3H), 1.34-1.23 (m, 4H), 1.18 (d, J=7.2 Hz, 3H).
Example 142. Synthesis of 1-octylnonyl 8-bromo-2-methyloctanoate

This example illustrates synthesis of:

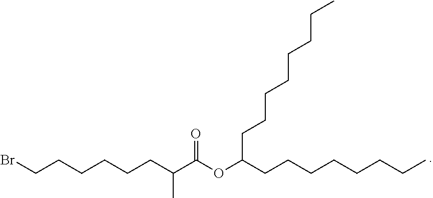 [SMILES: CCCCCCCCC(CCCCCCCC)OC(=O)C(C)CCCCCCBr]

To a stirred solution of 8-bromo-2-methyloctanoic acid (1 g, 4.22 mmol, 1 eq) in dichloromethane (15 ml), was added 4-(dimethylamino)pyridin-1-ium (1.04 g, 8.43 mmol, 2 eq) and {3-[cyano(ethyl)amino]propyl}dimethylazanium chloride (3.23 g, 16.9 mmol, 4 eq) and stirred for 15 min, then 9-heptadecanol (1.08 g, 4.22 mmol, 1 eq) was added. The reaction mixture was stirred at r.t. for 48 h. The progress of reaction was monitored by TLC (SM was consumed). Water (500 mL) was added to the reaction mixture and extracted with DCM (3×500 mL). The resulting organic layer was dried over Na₂SO₄, and concentrated under reduce pressure, and the crude was purified by flash column chromatography (SiO₂: 0-2% Ethyl acetate in Hexanes), to get a 1-octylnonyl 8-bromo-2-methyloctanoate (0.9 g, Yield=44.87%) as colorless liquid. ¹H-NMR (400 MHz, CDCl₃-d₃): δ 4.91-4.84 (m, 2H), 3.52 (t, J=6.8 Hz, 1H), 3.41 (t, J=6.8 Hz, 1H), 2.46-2.37 (m, 1H), 1.89-1.84 (m, 1H), 1.83-1.76 (m, 1H), 1.69-1.63 (m, 1H), 1.53-1.51 (m, 4H), 1.43-1.36 (m, 3H), 1.32-1.27 (m, 27H), 1.16 (d, J=6.8 Hz, 3H), 0.89 (d, J=6.8 Hz, 6H).
Example 143. Synthesis of heptadecan-9-yl 8-((2-hydroxyethyl)(8-(nonyloxy)-8-oxooctyl)amino)-2-methyloctanoate

This example illustrates synthesis of:

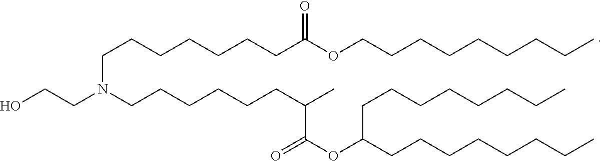 [SMILES: CCCCCCCCCOC(=O)CCCCCCCN(CCO)CCCCCCC(C)C(=O)OC(CCCCCCCC)CCCCCCCC]

To a stirred solution of compound 1-octylnonyl 8-bromo-2-methyloctanoate (0.8 g, 1.68 mmol) 1.0 eq) in acetonitrile (10.4 mL), was added N-ethylbis(isopropyl)amine (881 μL, 5.05 mmol, 3 eq) and nonyl 8-(2-hydroxyethylamino)octanoate (554 mg, 1.68 mmol, 1.0 eq) to it. The reaction mixture was stirred at 95° C. for 16 h. The progress of reaction was monitored by TLC (SM was consumed). The reaction mixture was concentrated to get the crude mixture and the crude was purified by flash column chromatography (SiO₂: 0-6% methanol in dichloromethane), to get 1-octylnonyl 8-{(2-hydroxyethyl)[7-(nonyloxycarbonyl)heptyl]amino}-2-methyloctanoate (410 mg, Yield=33.6%) as a colorless liquid. The compound was dissolved in dichloromethane (10 ml) and filtered through Hydrophobic PTFE Syringe Filter, 25 mm*0.45 μm by glass syringe to get desired compound ¹H-NMR (400 MHz, CDCl₃-d₃): δ 4.87-4.81 (m, 1H), 4.05 (t, J=6.8 Hz, 2H), 3.96 (bs, 2H), 3.10-2.98 (m, 6H), 2.42-2.34 (m, 1H), 2.29 (t, J=7.2 Hz, 2H), 1.80 (bs, 4H), 1.64-1.57 (m, 6H), 1.50-1.49 (m, 4H), 1.35-1.25 (m, 49H), 1.14 (d, J=6.8 Hz, 2H), 0.87 (t, J=6.8 Hz, 9H). ELSD analysis: Purity 97.35%, Calculated C₄₅H₉₀NO₅, [M+H]=724.68, Observed=724.55 (m/z, M+H⁺).
Example 144. Synthesis of nonyl 8-bromo-2-methyloctanoate

This example illustrates synthesis of:

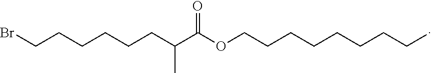

To a stirred solution of 8-bromo-2-methyloctanoic acid (0.8 g. 3.37 mmol, 1 eq) in dichloromethane (15 ml), was added 4-(dimethylamino)pyridin-1-ium (831 mg, 6.75 mmol, 2 eq) and {3-[cyano(ethyl)amino]propyl}dimethylazanium chloride (3.23 g, 16.9 mmol, 5 eq) and stirred for 15 min, then 1-nonanol (487 mg, 3.37 mmol, 1 eq) was added. The reaction mixture was stirred at r.t. for 48 h. The progress of reaction was monitored by TLC (SM was consumed). Water (500 mL) was added to the reaction mixture, and extracted with DCM (3×500 mL). The resulting organic layer was dried over Na₂SO₄, and concentrated under reduce pressure, and the crude was purified by flash column chromatography (SiO₂: 0-2% Ethyl acetate in Hexanes), to get a nonyl 8-bromo-2-methyloctanoate (0.8 g, Yield=65.2%) as a colorless liquid. ¹H-NMR (400 MHz, CDCl₃-d3): δ 4.07 (t, J=6.8 Hz, 2H), 3.54 (t, J=6.4 Hz, 1H), 3.41 (t, J=6.8 Hz, 1H), 2.45-2.40 (m, 1H), 1.88-1.84 (m, 1H), 1.79-1.74 (m, 1H), 1.68-1.60 (m, 1H), 1.46-1.39 (m, 3H), 1.35-1.29 (m, 18H), 1.15 (d, J=7.2 Hz, 3H), 0.89 (t, J=6.8 Hz, 3H).
Example 145. Synthesis of nonyl 8-((8-(heptadecan-9-yloxy)-8-oxooctyl)(2-hydroxyethyl)amino)-2-methyloctanoate

This example illustrates synthesis of:

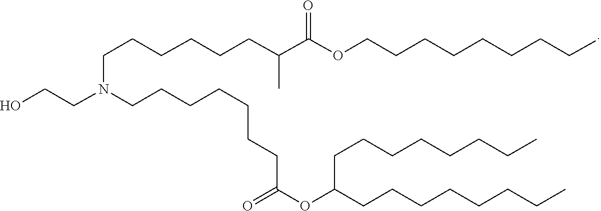

To a stirred solution of compound nonyl 8-bromo-2-methyloctanoate (0.7 g, 1.93 mmol, 1.0 eq) in acetonitrile (10 mL), was added (N-ethylbis(isopropyl)amine (404 μL, 2.31 mmol, 1.2 eq) and 1-octylnonyl 8-(2-hydroxyethylamino)octanoate (851 mg, 1.93 mmol, 1.0 eq) to it. The reaction mixture was stirred at 95° C. for 72 h. The progress of reaction was monitored by TLC/ELSD (59% product formation in reaction mixture by ELSD). The reaction mixture was concentrated to get the crude mixture and the crude was purified by flash column chromatography (SiO₂: 0-6% methanol in dichloromethane), to give nonyl 8-{(2-hydroxyethyl)[7-(1-octylnonyloxycarbonyl)heptyl]amino}-2-methyloctanoate (410 mg, Yield=29.39%) as a colorless liquid. The compound was dissolved in dichloromethane (10 ml), was added 5 eq of anhydrous K₂CO₃ under N₂ atmosphere and stirred reaction mixture Vigorously 2-3 hr then reaction mixture filtered by glass syringe using syringe filter (Hydrophobic PTFE Syringe Filter, 25 mm*0.45 μm) to get desired compound. ¹H-NMR (400 MHz, CDCl₃-d3): δ 4.89-4.82 (m, 1H), 4.05 (t, J=6.8 Hz, 2H), 3.59 (bs, 2H), 2.65-2.52 (bs, 4H), 2.45-2.36 (m, 1H), 2.27 (t, J=7.6 Hz, 2H), 1.64-1.58 (m, 6H), 1.50-1.49 (m, 8H), 1.31-1.26 (m, 50H), 1.14 (d, J=6.8 Hz, 3H), 0.89-0.85 (m, 9H). ELSD analysis: Purity 99.68%, Calculated C₄₅H₉₀NO₅, [M+H]=724.68, Observed=724.55 (m/z, M+H⁺).
Example 146. Synthesis of heptadecan-9-yl 8-((2-((4-(dimethylamino)butanoyl)oxy)ethyl)(8-(nonyloxy)-8-oxooctyl)amino)-2-methyloctanoate (Compound 38)

This example illustrates synthesis of:

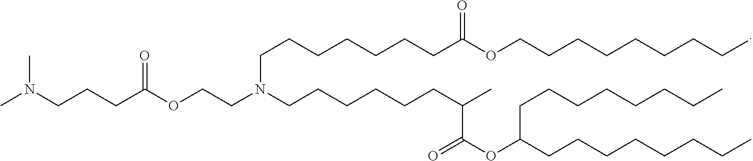

To a stirred solution of 4-(dimethylamino)butyric acid (194 mg, 1.16 mmol, 4 eq) in dichloromethane (5 ml), was added {3-[cyano(ethyl)amino]propyl}dimethylazanium chloride (334 mg, 1.74 mmol, 6 eq) and 4-(dimethylamino)pyridin-1-ium (106 mg, 870 μmol, 2 eq) to it, after stirred reaction mixture for 40 min, then 1-octylnonyl 8-{(2-hydroxyethyl)[7-(nonyloxycarbonyl)heptyl]amino}-2-methyloctanoate (210 mg, 290 μmol, 1.0 eq) was added. The reaction mixture was stirred at r.t. for 16 h. The progress of reaction was monitored by TLC/ELSD (38% product form in reaction mixture with 60% DMAP and other Acetonitrile soluble impurity by ELSD). The reaction mixture was quenched with brine solution and extracted with DCM (10 mL×2). The combined organic layer was dried over sodium sulphate, filtered and concentrated to get the crude mixture. The crude was diluted with pentane and washed with acetonitrile (10 mL×3). Pentane layer was evaporated and distilled (temp. below 30° C.) to get 1-octylnonyl 8-({2-[4-(dimethylamino)butyroxy]ethyl}[7-(nonyloxycarbonyl)heptyl]amino)-2-methyloctanoate (140 mg, Yield=57%) as colorless liquid. The compound was dissolved in dichloromethane (10 ml) and filtered through Hydrophobic PTFE Syringe Filter, 25 mm*0.45 μm by glass syringe to get desired compound. ¹H-NMR (400 MHz, CDCl₃-d₃): δ 4.88-4.82 (m, 1H), 4.11 (t, J=6.4 Hz, 2H), 4.05 (t, J=6.4 Hz, 2H), 2.67 (t, J=6.4 Hz, 2H), 2.45-2.39 (m, 4H), 2.37-2.33 (m, 4H), 2.30-2.27 (m, 8H), 1.85-1.80 (m, 2H), 1.62-1.57 (m, 5H), 1.50-1.49 (m, 4H), 1.44-1.34 (m, 4H) 1.25 (br, 48H), 1.13 (t, J=6.8 Hz, 2H), 0.88-0.85 (m, 12H). ELSD analysis: Purity 99.03%, Calculated C₅₁H₁₀₁N₂O₆, [M+H]=837.77, Observed=837.65 (m/z, M+H⁺).
Example 147. Synthesis of heptadecan-9-yl 8-((2-hydroxyethyl-1,1,2,2-d4)amino)octanoate

This example illustrates synthesis of:

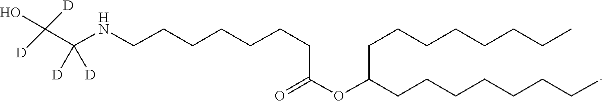

To a stirred solution of 2-amino(²H₄)ethanol 4 (250 mg, 3.84 mmol, 1.0 eq) in ethanol (50 mL) was added 1-octylnonyl 8-bromooctanoate 3 (1.95 g, 4.22 mmol, 1.1 eq) and N-ethylbis(isopropyl)amine (671 μL, 3.84 mmol, 1.0 eq) to it. The reaction was stirred at 55° C. for 16 h. The progress of reaction was monitored by TLC (SM was consumed). After completion the reaction, reaction mixture was concentrated under reduced pressure and the crude was purified by flash column chromatography (SiO₂: 0-12% Methanol in Dichloromethane) to give 1-octylnonyl 8-[2-hydroxy(²H₄)ethylamino]octanoate 5 (450 mg, yield 26.29%) as white semisolid. ELSD analysis: Purity 99.62%, Calculated C45H81O13 [M+H]=446.45, Observed=446.35 (m/z, M+H⁺).
Example 148. Synthesis of heptadecan-9-yl 8-((2-hydroxyethyl-1,1,2,2-d4)(8-(nonyloxy)-8-oxooctyl)amino)octanoate

This example illustrates synthesis of:

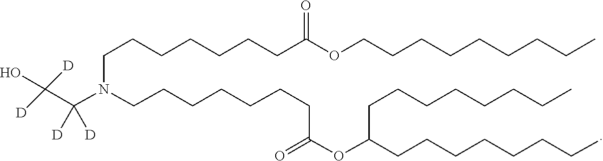

To a stirred solution of 1-octylnonyl 8-[2-hydroxy(²H₄)ethylamino]octanoate 5 (270 mg, 606 μmol, 1.0 eq) in acetonitrile (66.7 mL, 1.28 mol) was added nonyl 8-bromooctanoate 7 (233 mg, 666 μmol, 1.1 eq) and N-ethylbis(isopropyl)amine (317 μL, 1.82 mmol, 3 eq) to the reaction mass. The reaction was stirred at 90° C. for 16 h. The progress of reaction was monitored by ELSD/TLC (SM was consumed). After completion the reaction, reaction mixture was concentrated under reduced pressure and the crude was purified by flash column chromatography (SiO₂: 0-8% Methanol in Dichloromethane), to give 1-octylnonyl 8-{[2-hydroxy(²H₄)ethyl][7-(nonyloxycarbonyl)heptyl]amino}octanoate (154 mg, yield 35.6%) as yellow liquid. ¹H-NMR (400 MHz, CDCl₃)-δ 4.89-4.83 (m, 1H), 4.05 (t, J=6.8 Hz, 2H), 2.44-2.41 (m, 4H), 2.30-2.25 (m, 4H), 1.62-1.57 (m, 6H), 1.50-1.49 (m, 3H), 1.42 (m, 4H), 1.30-1.25 (m, 50H), 0.875 (m, 9H). ELSD analysis: Purity 99.73%, Calculated C₄₄H₈₄D₄NO₅ [M+H]=714.69, Observed=714.55 (m/z, M+H).
Example 149. Synthesis of heptadecan-9-yl 8-((2-((4-(dimethylamino)butanoyl)oxy)ethyl-1,1,2,2-d4)(8-(nonyloxy)-8-oxooctyl)amino)octanoate

This example illustrates synthesis of:

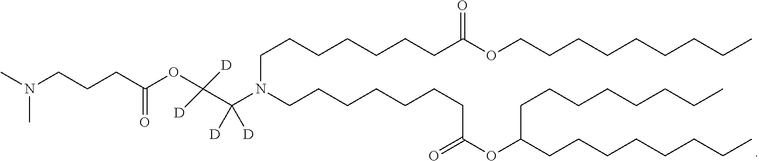 [SMILES: [2H]C([2H])(OC(=O)CCCN(C)C)C([2H])([2H])N(CCCCCCCC(=O)OCCCCCCCCC)CCCCCCCC(=O)OC(CCCCCCCC)CCCCCCCC]

To a stirred solution of 4-(dimethylamino)butyric acid (66.1 mg, 504 μmol, 4 eq) in dichloromethane (22.5 mL), was added {3-(dimethylamino)propyl]imino}methylidene)(ethyl)amine hydrochloride (96.6 mg, 504 μmol, 4 eq) and 4-(dimethylamino)pyridin-1-ium (30.8 mg, 252 μmol, 2 eq) and stirred for 15 min, then 1-octylnonyl 8-{[2-hydroxy(²H₄)ethyl][7-(nonyloxycarbonyl)heptyl]amino}octanoate (90 mg, 126 μmol, 1 eq) was added. The reaction mixture was stirred at r.t. for 16 h. The progress of reaction was monitored by ELSD/TLC (41% product form in reaction mixture with 54% DMAP and other Acetonitrile soluble impurity by ELSD). The reaction mixture was quenched with brine solution and extracted with DCM (2×10 mL). The combined organic layer was dried over sodium sulphate, filtered and concentrated to get the crude mixture. The crude was diluted with pentane and washed with acetonitrile (3×10 ml). Pentane layer was evaporated and distilled (temp. below 30° C.) to give 1-octylnonyl 8-({2-[4-(dimethylamino)butyroxy](²H₄)ethyl}[7-(nonyloxycarbonyl)heptyl]amino)octanoate (0.100 g. Yield=95.91%) as a colorless liquid. The compound was dissolved in dichloromethane (10 ml) and filtered through Hydrophobic PTFE Syringe Filter, 25 mm*0.45 μm by glass syringe to get desired compound. ¹H-NMR (400 MHz, CDCl₃)-δ 4.87-4.82 (m, 1H), 4.04 (t, J=6.8 Hz, 2H), 2.56-2.53 (m, 2H), 2.48 (t, J=7.6 Hz, 4H), 2.42-2.37 (m, 7H), 2.30-2.28 (4H), 1.95-1.88 (m, 2H), 1.64-1.57 (m, 6H), 1.50-1.42 (m, 9H), 1.30-1.25 (m, 48H), 0.88-0.85 (m, 9H). ELSD analysis: Purity 99.06%, Calculated C₅₀H₉₄D₄N₂O₆, [M+H]=827.78, Observed=827.75 (m/z, M+H⁺).
Example 150. Synthesis of 2-(dodecylamino)ethan-1-ol

This example illustrates synthesis of:

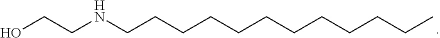 [SMILES: CCCCCCCCCCCCNCCO]

To a stirred solution of 2-aminoethan-1-ol (1 g, 16.4 mmol, 1.0 eq) in ethanol (25 mL) was added 1-bromododecane (4.08 g, 16.4 mmol, 1.0 eq) and ethylbis(propan-2-yl)amine (2.54 g, 19.6 mmol, 1.2 eq). The reaction was stirred at 55° C. for 16 h. The progress of reaction was monitored by TLC/ELSD (41.7% product form in reaction mixture by ELSD). After completion the reaction, reaction mixture was concentrated under reduced pressure and the crude was purified by flash column chromatography (SiO₂: 0-12% Methanol in Dichloromethane) to give 2-(dodecylamino)ethan-1-ol (1.3 g, Yield=34.6%) as a white semisolid. ELSD analysis: Purity 98.58%, Calculated C₁₄H₃₂NO, [M+H]=230.25, Observed=230.30 (m/z, M+H⁺).
Example 151. Synthesis of heptadecan-9-yl 8-(dodecyl(2-hydroxyethyl)amino)octanoate

This example illustrates synthesis of:

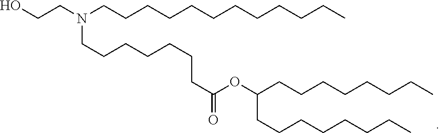 [SMILES: CCCCCCCCCCCCN(CCO)CCCCCCCC(=O)OC(CCCCCCCC)CCCCCCCC]

To a stirred solution of 2-(dodecylamino)ethan-1-ol (450 mg, 1.96 mmol, 1.0 eq) in acetonitrile (4.5 mL), was added ethylbis(propan-2-yl)amine (761 mg, 5.88 mmol, 3 eq) and heptadecan-9-yl 8-bromooctanoate (815 mg, 1.77 mmol, 0.9 eq). The reaction was stirred at 95° C. for 16 h. The progress of reaction was monitored by ELSD/TLC (58.6% product form in reaction mixture by ELSD). The reaction mixture was concentrated under reduced pressure and the crude was purified by flash column chromatography (SiO₂: 0-8% Methanol in Dichloromethane), to get heptadecan-9-yl 8-[dodecyl(2-hydroxyethyl)amino]octanoate (330 mg, Yield=27.6%) as a colorless liquid. The compound was dissolved in dichloromethane (10 ml) was added 5 eq of anhydrous K₂CO₃ under N₂ atmosphere and stirred reaction mixture vigorously 2-3 hr then reaction mixture filtered by glass syringe using syringe filter (Hydrophobic PTFE Syringe Filter, 25 mm*0.45 μm) to get desired compound. ¹H-NMR (400 MHz, CDCL₃-d3): δ 4.88-4.82 (m, 1H), 3.85 (br, 2H), 2.97 (bs, 2H), 2.87 (br, 4H), 2.27 (t, J=7.6 Hz, 2H), 1.70 (br, 4H), 1.60 (t, J=6.8 Hz, 2H), 1.50-1.49 (m, 4H), 1.33-1.25 (m, 49H), 0.88-0.85 (m, 9H). ELSD analysis: Purity 99.55%, Calculated C₃₉H₈₀NO₃, [M+H]=610.62, Observed=610.45 (m/z, M+H⁺).
Example 152. Synthesis of undecyl 6-(dodecyl(2-hydroxyethyl)amino)hexanoate

This example illustrates synthesis of:

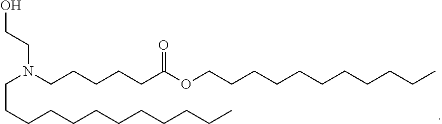 [SMILES: CCCCCCCCCCCCN(CCO)CCCCCC(=O)OCCCCCCCCCCC]

To a stirred solution of 2-(dodecylamino)ethan-1-ol (1 g, 4.36 mmol, 1.0 eq) in Acetonitrile (25 mL), was added N-ethylbis(isopropyl)amine (1.69 g, 3 eq., 13.1 mmol) and undecyl 6-bromohexanoate 3 (1.52 g, 4.36 mmol, 1.0 eq). The reaction was stirred at 95° C. for 16 h. The progress of reaction was monitored by ELSD/TLC (SM was consumed). After completion the reaction, reaction mixture was concentrated under reduced pressure and the crude was purified by flash column chromatography (SiO₂: 0-8% Methanol in Dichloromethane) to give undecyl 6-[dodecyl(2-hydroxyethyl)amino]hexanoate (470 mg, Yield=21.7%) as a colorless liquid. The compound was dissolved in dichloromethane (10 ml) and filtered through Hydrophobic PTFE Syringe Filter, 25 mm*0.45 μm by glass syringe to get desired compound. ¹H-NMR (400 MHz, DMSO-d6): δ 4.23 (t, J=5.2 Hz, 1H), 3.98 (t, J=6.4 Hz, 2H), 3.41-3.36 (m, 2H), 2.39 (t, J=6.8 Hz, 2H), 2.34 (t, J=7.2 Hz, 4H), 2.25 (t, J=7.2 Hz, 2H), 1.54-1.49 (m, 4H), 1.34-1.23 (m, 40H), 0.88-0.85 (t, J=6.4 Hz, 6H). ELSD analysis: Purity 99.33%, Calculated C₃₁H₆₄NO₃, [M+H]=498.49, Observed=498.50 (m/z, M+H⁺).
Example 153. Synthesis of heptadecan-9-yl 8-((2-hydroxyethyl)(8-(nonyloxy)-8-oxooctyl)amino)octanoate

This example illustrates synthesis of:

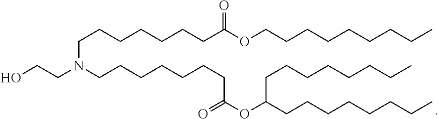 [SMILES: CCCCCCCCCOC(=O)CCCCCCCN(CCO)CCCCCCCC(=O)OC(CCCCCCCC)CCCCCCCC]

To a stirred solution of heptadecan-9-yl 8-[(2-hydroxyethyl)amino]octanoate 5 (2.1 g, 4.75 mmol, 1.0 eq) in acetonitrile (38.2 ml), was added nonyl 8-bromooctanoate 7 (1.83 g, 5.23 mmol, 1.1 eq) and N-ethylbis(isopropyl)amine (2.49 ml, 14.3 mmol, 3 eq) to it. The reaction was stirred at 90° C. for 16 h. The progress of reaction was monitored by TLC/ELSD (90.15% product form in reaction mixture by ELSD). The reaction mixture was concentrated under reduced pressure and the crude was purified by flash column chromatography (SiO₂: 0-8% Methanol in Dichloromethane) to give the desired heptadecan-9-yl 8-[(2-hydroxyethyl)[8-(nonyloxy)-8-oxooctyl]amino]octanoate (2.1 g. Yield=62.2%) as a yellow liquid. ¹H-NMR (400 MHz, CDCl₃-d3): δ 4.88-4.81 (m, 1H), 4.04 (t, J=6.8 Hz, 2H), 3.99 (bs, 2H), 3.14-3.02 (m, 5H) 2.30-2.25 (m, 4H), 1.82 (m, 4H), 1.64-1.59 (m, 6H), 1.50-1.49 (m, 4H), 1.35-1.25 (m, 50H), 0.88-0.85 (m, 9H). ELSD analysis: Purity 99.19%, Calculated C44H88NO5, [M+H]=710.67, Observed=710.50 (m/z, M+H⁺).
Example 154. Synthesis of heptadecan-9-yl 8-((2-fluoroethyl)(8-(nonyloxy)-8-oxooctyl)amino)octanoate

This example illustrates synthesis of:

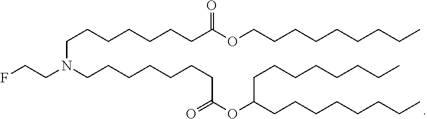 [SMILES: CCCCCCCCCOC(=O)CCCCCCCN(CCF)CCCCCCCC(=O)OC(CCCCCCCC)CCCCCCCC]

To a stirred solution of 1-octylnonyl 8-{(2-hydroxyethyl)[7-(nonyloxycarbonyl)heptyl]amino}octanoate (1 g, 1.41 mmol, 1.0 eq) in dichloromethane (22.2 ml) was added dropwise N,N-diethyl(trifluorothio)amine (946 μL, 7.04 mmol, 5 eq) at 0° C. The reaction mixture was stirred at r.t. for 4 h. The progress of reaction was monitored by TLC/ELSD (91% product form in reaction mixture by ELSD). The mixture was quenched with sat aq NaHCO₃, and extracted with DCM (3×50 mL). The resulting organic layer was dried over Na₂SO₄, and concentrated under reduce pressure, and the crude was purified by flash column chromatography (SiO₂: 0-4% Ethyl acetate in Hexane), to get 1-octylnonyl 8-{(2-fluoroethyl)[7-(nonyloxycarbonyl)heptyl]amino}octanoate (430 mg, Yield=42.88%) as a pale yellow liquid. The compound was dissolved in dichloromethane (10 ml) and filtered through Hydrophobic PTFE Syringe Filter, 25 mm*0.45 μm by glass syringe to get desired compound. ¹H-NMR (400 MHz, CDCl₃-d₃): δ 4.89-4.83 (m, 1H), 4.56-4.45 (br, 2H), 4.05 (t, J=6.8 Hz, 2H), 2.76 (br, 2H), 2.49 (br, 4H), 2.30-2.25 (m, 4H), 1.62-1.57 (m, 6H), 1.50-1.43 (m, 6H), 1.32-1.25 (m, 48H), 0.89-0.85 (m, 9H). ¹⁹F-NMR (400 MHz, CDCl₃-d₃): δ −219.224. ELSD analysis: Purity 99.29%, Calculated C₄₄H₈₆FNO₄, [M+H]=712.66, Observed=712.50 (m/z, M+H⁺).
Example 155. Synthesis of di(heptadecan-9-yl) 8,8′-((2-hydroxyethyl)azanediyl)dioctanoate

This example illustrates synthesis of:

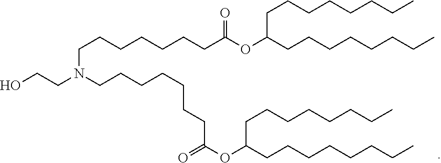 [SMILES: CCCCCCCCC(CCCCCCCC)OC(=O)CCCCCCCN(CCO)CCCCCCCC(=O)OC(CCCCCCCC)CCCCCCCC]

To a stirred solution of 2-aminoethan-1-ol (794 mg, 13 mmol, 1.0 eq.) in acetonitrile (50 mL), was added heptadecan-9-yl 8-bromooctanoate (6 g, 13 mmol, 1.0 eq.) and ethylbis(propan-2-yl)amine (2.72 mL, 15.6 mmol, 1.2 eq.). The reaction was stirred at 55° C. for 4 days. The reaction mixture was concentrated under reduced pressure and the crude was purified by flash column chromatography (silica gel, 0-5% gradient of methanol in dichloromethane) to get 1-octylnonyl 8-{(2-hydroxyethyl)[7-(1-octylnonyloxycarbonyl)heptyl]amino}octanoate (850 mg, Yield=7.95%) as a brown liquid. (850 mg) as a salt was dissolved in dichloromethane (10 mL) and added anhydrous potassium carbonate (5 eq.) under nitrogen atmosphere and stirred vigorously for 2 h. Then, reaction mixture was filtered through hydrophobic PTFE syringe filter (25 mm×0.45 μm) using glass syringe to get 1-octylnonyl 8-{(2-hydroxyethyl)[7-(1-octylnonyloxycarbonyl)heptyl]amino}octanoate (530 mg, Yield=4.96%) as a brown liquid. ¹H-NMR (400 MHz, CDCl₃-d3): δ 4.86 (quin, J=6.0 Hz, 2H), 3.51 (t, J=5.2 Hz, 2H), 2.56 (t, J=5.2 Hz, 2H), 2.42 (t, J=7.6 Hz, 4H), 2.27 (t, J=7.6 Hz, 4H), 1.68-1.55 (m, 6H), 1.54-1.45 (m, 8H), 1.44-1.37 (m, 4H), 1.35-1.17 (m, 58H), 0.87 (t, J=6.8 Hz, 12H). ELSD analysis: Purity 98.74%, Calculated C₅₂H₁₀₄NO₅, [M+H]=822.79, Observed=822.95 (m/z, M+H⁺).
Example 156. Synthesis of bis(9-methyldecyl) 8,8′-((2-hydroxyethyl)azanediyl)dioctanoate

This example illustrates synthesis of:

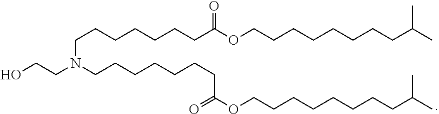 [SMILES: CC(C)CCCCCCCCOC(=O)CCCCCCCN(CCO)CCCCCCCC(=O)OCCCCCCCCC(C)C]

To a stirred solution of 9-methyldecyl 8-bromooctanoate (3.7 g, 9.8 mmol) in acetonitrile (25 mL), N-ethylbis(isopropyl)amine (4.28 mL, 2.5 eq., 24.5 mmol) and 2-aminoethanol (599 mg, 9.8 mmol) was added and reaction mixture was stirred at 55° C. for 16 h. TLC showed starting materials were consumed and formed new spots. The reaction mass was concentrated and the residue was purified by flash column chromatography (silica gel, 0-10% gradient of methanol in DCM) to obtain 9-methyldecyl 8-{(2-hydroxyethyl)[7-(9-methyldecyloxycarbonyl) heptyl]amino}(0.55 g) as a pale yellow liquid. The compound (0.55 mg) was obtained after column chromatography was dissolved in dichloromethane (10 mL) and added anhydrous potassium carbonate (5 eq.) under nitrogen atmosphere and stirred vigorously for 2 h. Then, reaction mixture was filtered through hydrophobic PTFE syringe filter (25 mm×0.45 μm) using glass syringe to get bis(9-methyldecyl) 8,8′-((2-hydroxyethyl)azanediyl)dioctanoate (0.5 mg, Yield=7%) as a pale yellow liquid. ¹H NMR (400 MHz, CDCl₃-d3): δ 4.05 (t, J=6.8 Hz, 4H), 3.62-3.60 (m, 2H), 2.70-2.67 (m, 2H), 2.58-2.54 (m, 4H), 2.28 (t, J=7.6 Hz, 4H), 1.64-1.56 (m, 8H), 1.54-1.46 (m, 6H), 1.31-1.25 (m, 34H), 1.16-1.12 (m, 3H), 0.86 (d, J=6.8 Hz, 12H). ELSD analysis: Purity 99.88%, Calculated for C₄₀H₈₀NO₅, [M+H]=654.60, Observed=654.45 (m/z, M+H⁺).
Example 157. Synthesis of ((2-hydroxyethyl)azanediyl)bis(heptane-7,1-diyl)bis(2-octyldecanoate)

This example illustrates synthesis of:

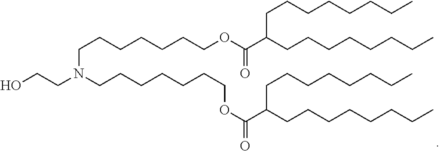 [SMILES: CCCCCCCCC(CCCCCCCC)C(=O)OCCCCCCCN(CCO)CCCCCCCOC(=O)C(CCCCCCCC)CCCCCCCC]

To a stirred solution of 2-aminoethanol (185 mg, 3.03 mmol) in acetonitrile (15 mL), was added 7-bromoheptyl 2-octyldecanoate (1.4 g. 3.03 mmol) and N-ethylbis(isopropyl)amine (636 μL, 1.2 eq., 3.64 mmol). The mixture was stirred at 55° C. for 16 h. After complete consumption of starting materials (monitored by TLC), reaction mixture was concentrated under reduced pressure. The residue was purified by flash column chromatography (silica gel, 0-12% gradient of methanol in dichloromethane) to obtain 7-{(2-hydroxyethyl)[7-(1-octylnonylcarbonyloxy)heptyl]amino}heptyl2-octyldecanoate (185 mg) as a pale-yellow liquid. The compound (185 mg) was obtained after column chromatography was dissolved in dichloromethane (10 mL) and added anhydrous potassium carbonate (5 eq.) under a nitrogen atmosphere and stirred vigorously for 2 h. Then, the reaction mixture was filtered through a hydrophobic PTFE syringe filter (25 mm×0.45 μm) using a glass syringe to get 7-{(2-hydroxyethyl)[7-(1-octylnonylcarbonyloxy)heptyl]amino}heptyl 2-octyldecanoate (170 mg, Yield=7%) as a pale yellow liquid. ¹H NMR (400 MHz, CDCl₃-d3): δ 4.05 (t, J=6.8 Hz, 4H), 3.51 (t, J=5.2 Hz, 2H), 2.56 (t, J=5.2 Hz, 2H), 2.43 (t, J=7.2 Hz, 4H), 2.35-2.28 (m, 2H), 1.63-1.55 (m, 12H), 1.44-1.39 (m, 8H), 1.34-1.22 (m, 56H), 0.91-0.84 (m, 12H). ELSD analysis: Purity 98.28%, Calculated for C₅₂H₁₀₄NO₅, [M+H]=822.78, Observed=822.95 (m/z, M+H⁺).
Example 158. Synthesis of ((2-hydroxyethyl)azanediyl)bis(heptane-7,1-diyl)bis(decanoate)

This example illustrates synthesis of:

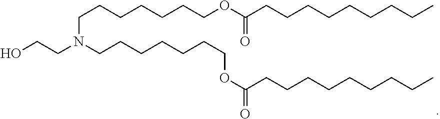 [SMILES: CCCCCCCCCC(=O)OCCCCCCCN(CCO)CCCCCCCOC(=O)CCCCCCCCC]

To a solution of 7-bromoheptyl decanoate (3 g, 8.59 mmol)) in acetonitrile (20 mL), was added N-ethylbis(isopropyl)amine (3.75 mL, 2.5 eq., 21.5 mmol) and 2-aminoethanol (525 mg, 8.59 mmol) at room temperature. Reaction mixture was stirred at 55° C. for 16 h. After complete consumption of starting materials, reaction mass concentrated. The crude thus obtained was purified by flash column chromatography (silica gel, 0-3% gradient of methanol in DCM) to obtain ((2-hydroxyethyl)azanediyl)bis(heptane-7,1-diyl)bis(decanoate)(0.2 g, Yield=4%) as a colorless liquid. The compound was dissolved in dichloromethane (5 mL) under a nitrogen atmosphere and filtered through a hydrophobic PTFE syringe filter (25 mm×0.45 μm) using a glass syringe to give the desired compound. ¹H-NMR (400 MHz, CDCl₃-d3): δ 4.05 (t, J=6.8 Hz, 4H), 3.55 (t, J=4.8 Hz, 2H), 2.61 (t, J=4.8 Hz, 2H), 2.48 (t, J=6.8 Hz, 4H), 2.28 (t, J=7.6 Hz, 4H), 1.65-1.55 (m, 8H), 1.52-1.41 (m, 4H), 1.35-1.21 (m, 37), 0.87 (t, J=7.2 Hz, 6H). ELSD analysis: Purity 99.77%, Calculated C₃₆H₇₂NO₅, [M+H]=598.54, Observed=598.65 (m/z, M+H⁺).
Example 159. Synthesis of ((2-hydroxyethyl)azanediyl)bis(heptane-7,1-diyl)bis(10-methylundecanoate)

This example illustrates synthesis of:

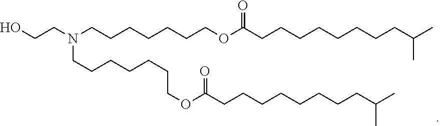 [SMILES: CC(C)CCCCCCCCC(=O)OCCCCCCCN(CCO)CCCCCCCOC(=O)CCCCCCCCC(C)C]

To a stirred solution of 7-bromoheptyl 10-methylundecanoate (2.7 g, 7.15 mmol) in acetonitrile (20 mL), was added N-ethylbis(isopropyl)amine (3.12 mL, 2.5 eq., 17.9 mmol) and 2-aminoethanol (437 mg, 7.15 mmol). Reaction mixture was stirred at 55° C. for 16 h. After complete consumption of starting materials, reaction mass concentrated. The crude thus obtained was purified by flash column chromatography (silica gel, 0-3% gradient of methanol in DCM) to obtain ((2-hydroxyethyl)azanediyl)bis(heptane-7,1-diyl)bis(10-methylundecanoate)(250 mg, Yield=5.34%) as a colourless liquid. The compound was dissolved in dichloromethane (5 mL) under a nitrogen atmosphere and filtered through a hydrophobic PTFE syringe filter (25 mm×0.45 μm) using a glass syringe to give the desired compound. ¹H NMR (400 MHz, CDCL₃-d3): δ 4.05 (t, J=6.8 Hz, 4H), 3.53 (t, J=5.2 Hz, 2H), 2.56 (t, J=5.6 Hz, 2H), 2.43 (t, J=7.6 Hz, 4H), 2.28 (t, J=7.6 Hz, 4H), 1.63-1.58 (m, 8H), 1.54-1.39 (m, 6H), 1.33-1.25 (m, 32H), 1.15-1.11 (m, 4H), 0.86 (d, J=6.8 Hz, 12H). ELSD analysis: Purity 99.53%, Calculated C₄₀H₈₀NO₅, [M+H]=654.60, Observed=654.75 (m/z, M+H⁺).
Example 160. Synthesis of diundecyl 6,6′-((2-hydroxyethyl)azanediyl)dihexanoate

This example illustrates synthesis of:

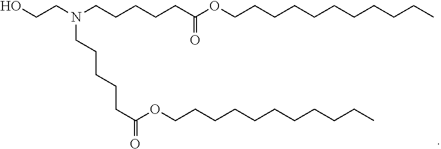 [SMILES: CCCCCCCCCCCOC(=O)CCCCCN(CCO)CCCCCC(=O)OCCCCCCCCCCC]

To a stirred solution of undecyl 6-bromohexanoate (5 g, 14.3 mmol) in acetonitrile (20 mL) was added N-ethylbis(isopropyl)amine (5.55 g, 3 eq., 42.9 mmol) and 2-amino-1-ethanol (874 mg, 14.3 mmol, 1.0 eq.). The reaction mixture was stirred at 55° C. for 16 h. The progress of reaction was monitored by ELSD and TLC. The reaction mixture was concentrated and crude thus obtained was purified by flash column chromatography (silica gel, 0-5% gradient of methanol in dichloromethane) to get undecyl 6-{(2-hydroxyethyl)[5-(undecyloxycarbonyl)pentyl]amino}hexanoate (0.25 g) as a colorless liquid.

Desalting procedure: The compound (250 mg) was obtained after column chromatography was dissolved in dichloromethane (10 mL) and added anhydrous potassium carbonate (5 eq.) under nitrogen atmosphere and stirred vigorously for 2 h. Then, reaction mixture was filtered through hydrophobic PTFE syringe filter (25 mm×0.45 μm) using glass syringe to get undecyl 6-{(2-hydroxyethyl)[5-(undecyloxycarbonyl)pentyl]amino}hexanoate (0.2 g, Yield=2.5%) as pale-yellow liquid. ELSD analysis: Purity 99.64%, Calculated: C₃₆H₇₂NO₅, [M+H]=598.54, Observed=598.65 (m/z, M+H⁺). ¹H-NMR (400 MHz, CDCl₃-d3): δ 4.05 (t, J=6.8 Hz, 4H), 3.51 (t, J=5.2 Hz, 2H), 3.21-2.72 (bs, 1H), 2.56 (t, J=5.2 Hz, 2H), 2.43 (t, J=7.4 Hz, 4H), 2.30 (t, J=7.6 Hz, 4H), 1.67-1.57 (m, 8H), 1.44 (quin, J=7.6 Hz, 4H), 1.37-1.21 (m, 35H), 0.87 (t, J=6.8 Hz, 6H).

Example 161 Synthesis of dinonyl 6,6′-((2-hydroxyethyl)azanediyl)dihexanoate

This example illustrates synthesis of:

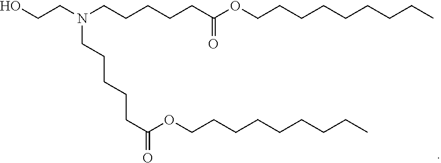 [SMILES: CCCCCCCCCOC(=O)CCCCCN(CCO)CCCCCC(=O)OCCCCCCCCC]

To a stirred solution of nonyl 6-bromohexanoate (1 g, 3.11 mmol) in acetonitrile (10 mL), was added N-ethylbis(isopropyl)amine (1.21 g, 3 eq., 9.34 mmol) and 2-aminoethanol (190 mg, 3.11 mmol). Reaction mixture was stirred at 55° C. for 16 h. The progress of reaction was monitored by ELSD and TLC. After complete consumption of starting materials, reaction mixture was concentrated. The crude thus obtained was purified by flash column chromatography (silica gel, 0-8% gradient of methanol in DCM) to obtain nonyl 6-{(2-hydroxyethyl)[5-(nonyloxycarbonyl)pentyl]amino}hexanoate (0.5 g, Yield=29.65%) as a colorless liquid.

Desalting procedure: The compound (0.5 g) was obtained after column chromatography was dissolved in dichloromethane (10 mL) and added anhydrous potassium carbonate (5 eq.) under nitrogen atmosphere and stirred vigorously for 2 h. Then, reaction mixture was filtered through hydrophobic PTFE syringe filter (25 mm×0.45 μm) using glass syringe to get nonyl 6-{(2-hydroxyethyl)[5-(nonyloxycarbonyl)pentyl]amino}hexanoate (260 mg, Yield=15%) as a colorless liquid. ¹H-NMR (400 MHz, CDCl₃-d3): δ 4.05 (t, J=6.4 Hz, 4H), 3.52 (t, J=5.2 Hz, 2H), 2.56 (t, J=5.2 Hz, 2H), 2.44 (t, J=7.6 Hz, 4H), 2.30 (t, J=7.6 Hz, 4H), 1.67-1.55 (m, 8H), 1.51-1.41 (m, 4H), 1.38-1.21 (m, 28H), 0.87 (t, J=6.8 Hz, 6H). ELSD analysis: Purity 97.44%, Calculated: C₃₂H₆₄NO₅, [M+H]=542.48, Observed=542.55 (m/z, M+H⁺).

Example 162. Synthesis of di(heptadecan-9-yl) 6,6′-((2-hydroxyethyl)azanediyl)dihexanoate

This example illustrates synthesis of:

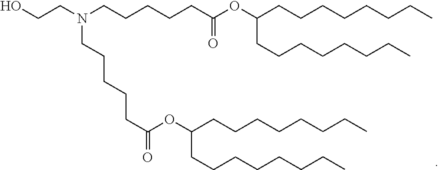 [SMILES: CCCCCCCCC(CCCCCCCC)OC(=O)CCCCCN(CCO)CCCCCC(=O)OC(CCCCCCCC)CCCCCCCC]

To a stirred solution of 1-octylnonyl 6-bromohexanoate (5.68 g, 13.1 mmol, 1.0 eq) in acetonitrile (20 mL) was added N-ethylbis(isopropyl)amine (4.84 mL, 26.2 mmol, 2.0 eq) and 2-aminoethanol (0.8 g, 13.1 mmol, 1.0 eq). The reaction mixture was stirred at 55° C. for 16 h. The progress of the reaction was monitored by ELSD and TLC. After 16 h, TLC shows the formation of a new spot and SM consumed. The reaction mixture was concentrated and the crude thus obtained was purified by flash column chromatography (silica gel, 0-5% gradient of methanol in dichloromethane) to get di(heptadecan-9-yl) 6,6′-((2-hydroxyethyl)azanediyl)dihexanoate (1.5 g, Yield=14.91%) as a colorless liquid. The compound (350 mg) was obtained after column chromatography was dissolved in dichloromethane (10 mL) and added anhydrous potassium carbonate (5 eq.) under nitrogen atmosphere and stirred vigorously for 2 h. Then, reaction mixture was filtered through hydrophobic PTFE syringe filter (25 mm×0.45 μm) using glass syringe to get di(heptadecan-9-yl) 6,6′-((2-hydroxyethyl)azanediyl)dihexanoate (0.3 g, Yield=14.91%) as a colorless liquid. The compound was dissolved in dichloromethane (5 mL) under a nitrogen atmosphere and filtered through a hydrophobic PTFE syringe filter (25 mm×0.45 μm) using a glass syringe to give the desired compound. ¹H-NMR (400 MHz, CDCl₃-d3): δ 4.86 (quin, J=6.4 Hz, 1H), 3.51 (t, J=5.2 Hz, 2H), 2.92 (b, 1H), 2.56 (t, J=5.2 Hz, 2H), 2.43 (t, J=7.2 Hz, 4H), 2.28 (t, J=7.6 Hz, 4H), 1.67-1.56 (m, 4H), 1.50-1.36 (m, 12H), 1.36-1.25 (m, 52H), 0.87 (t, J=6.8 Hz, 12H). ELSD analysis: Purity 99.70%, Calculated C₄₈H₉₅NO₅, [M+H]=766.72, Observed=766.85 (m/z, M+H⁺).
Example 163. Synthesis of 1-fluoroheptadecan-9-yl 8-((4-hydroxybutyl)(8-(nonyloxy)-8-oxooctyl)amino)octanoate (Compound 31)

This example illustrates synthesis of:

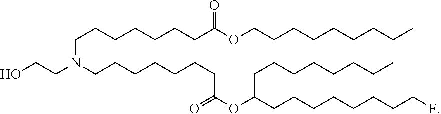 [SMILES: CCCCCCCCCOC(=O)CCCCCCCN(CCO)CCCCCCCC(=O)OC(CCCCCCCC)CCCCCCCCF]

The compound was synthesized via a method similar to example 162.
Example 164. Synthesis of heptadecan-9-yl 8-((2-hydroxyethyl-1,1,2,2-d4)(8-(nonyloxy)-8-oxooctyl)amino)octanoate (Compound 39)

This example illustrates synthesis of:

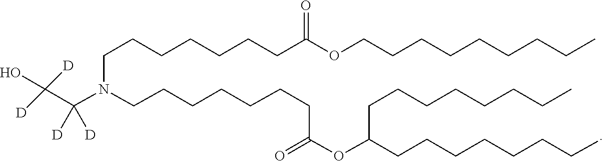

The compound was synthesized via a method similar to example 159.
Example 165. Synthesis of heptadecan-9-yl 8-((2-((4-(dimethylamino)butanoyl)oxy)ethyl-1,1,2,2-d4)(8-(nonyloxy)-8-oxooctyl)amino)octanoate (Compound 40)

This example illustrates synthesis of:

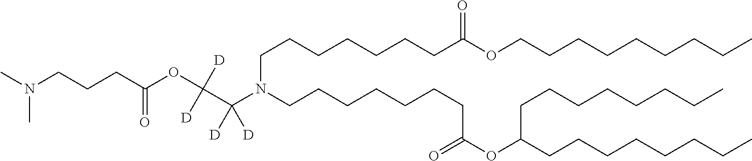 [SMILES: [2H]C([2H])(OC(=O)CCCN(C)C)C([2H])([2H])N(CCCCCCCC(=O)OCCCCCCCCC)CCCCCCCC(=O)OC(CCCCCCCC)CCCCCCCC]

The compound was synthesized via a method similar to example 159.
Example 166. Synthesis of Compound 52: 7-((7-((2-(7-fluoroheptyl) decanoyl)oxy)heptyl)(2-hydroxyethyl)amino)heptyl 10-methylundec-9-enoate

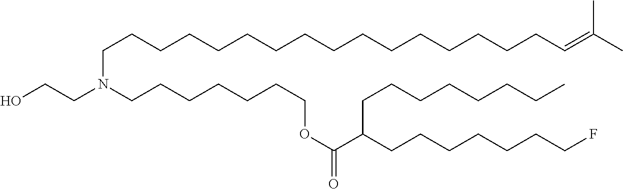 [SMILES: CCCCCCCCC(CCCCCCCF)C(=O)OCCCCCCCN(CCO)CCCCCCCCCCCCCCCCC=C(C)C]

To a stirred solution of 7-(2-hydroxyethylamino)heptyl 2-(7-fluoroheptyl) decanoate (450 mg, 1.01 mmol, 1.0 eq.) in acetonitrile (10 mL), was added N-ethylbis(isopropyl)amine (529 μL, 3.03 mmol, 3 eq.) and reaction mixture was stirred at room temperature for 10 minutes. Then, 7-bromoheptyl 10-methyl-9-undecenoate (379 mg, 1.01 mmol, 1.0 eq.) was added and reaction mixture was stirred at 100° C. for 16 h. Reaction was monitored by TLC. After starting materials were consumed, reaction mixture was concentrated under reduced pressure. The crude was purified by flash column chromatography (silica gel, 0-10% methanol in DCM) to get 7-({7-[1-(7-fluoroheptyl)nonylcarbonyloxy]heptyl}(2-hydroxyethyl)amino)heptyl 10-methyl-9-undecenoate (400 mg) with ELSD purity is 97% Further, compound obtain after column chromatography was purified by preparative HPLC to get 7-({7-[1-(7-fluoroheptyl)nonylcarbonyloxy]heptyl}(2-hydroxyethyl)amino)heptyl 10-methyl-9-undecenoate (75 mg, yield=10%) as a colourless liquid. ¹H-NMR (400 MHz, CDCL₃-d3): δ 5.10 (t, J=6.8 Hz, 1H), 4.48 (t, J=6.0 Hz, 1H), 4.36 (t, J=6.0 Hz, 1H), 4.07-4.03 (m, 4H), 3.53 (b, 2H), 2.58 (b, 2H), 2.45 (b, 4H), 2.34-2.26 (m, 3H), 1.95-1.92 (m, 2H), 1.73-1.70 (m, 1H), 1.68 (s, 3H), 1.62-1.59 (m, 12H), 1.44-1.39 (m, 4H), 1.32-1.25 (42H), 0.89-0.85 (m, 3H). ¹⁹F-NMR (375 MHz, CDCL₃-d3): δ −218.02. ELSD analysis: Purity 98.94%, Calculated C₄₅H₈₇FNO₅, [M+H⁺]=740.65, Observed=740.45 (m/z, M+H⁺).

Example 167. Synthesis of Compound 53:1-fluoroheptadecan-9-yl 8-((2-(dimethylamino)ethyl)(8-((9-methyldecyl)oxy)-8-oxooctyl)amino)octanoate
Step 1: Synthesis of 9-methyldecyl 8-((2-(dimethylamino)ethyl)amino)octanoate

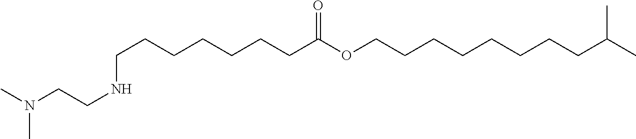 [SMILES: CC(C)CCCCCCCCOC(=O)CCCCCCCNCCN(C)C]

To a stirred solution of 2-(dimethylamino)ethanamine (350 mg, 3.97 mmol, 1.0 eq) in acetonitrile (90 mL), was added 9-methyldecyl 8-bromooctanoate (1.5 g, 3.97 mmol, 1.0 eq) and N-ethylbis(isopropyl)amine (2.0 mL, 11.9 mmol, 3.0 eq) to it. The reaction was stirred at 55° C. for 4 days. The progress of reaction was monitored by ELSD/TLC (70% product form in reaction mixture by ELSD). The reaction mixture was concentrated under reduced pressure and the crude was purified by flash column chromatography (SiO₂: 0-10% Methanol in Dichloromethane) to get 9-methyldecyl 8-[2-(dimethylamino)ethylamino]octanoate (650 mg, Yield=42.83%) as a brown solid. ELSD analysis: Purity 99.22%, Calculated C₂₃H₄₉N₂O₂, [M+H+]=385.37, Observed=385.25 (m/z, M+H+).
Step 2: Synthesis of 1-fluoroheptadecan-9-yl 8-((2-(dimethylamino)ethyl)(8-((9-methyldecyl)oxy)-8-oxooctyl)amino)octanoate

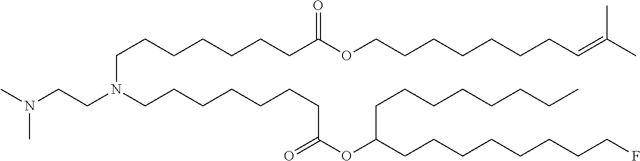 [SMILES: CCCCCCCCC(CCCCCCCCF)OC(=O)CCCCCCCN(CCCCCCCC(=O)OCCCCCCCC=C(C)C)CCN(C)C]

To a stirred solution of 9-fluoro-1-octylnonyl 8-bromooctanoate (0.450 g, 940 μmol, 1.0 eq) in acetonitrile (10 mL), was added N-ethylbis(isopropyl)amine (180.4 μL, 1030 μmol, 1.1 eq.) and 9-methyldecyl 8-[2-(dimethylamino)ethylamino]octanoate (360.8 mg, 940 μmol, 1.0 eq) to it. The reaction mixture was stirred at 95° C. for 48 h. The progress of reaction was monitored by TLC/ELSD (39% product formation in reaction mixture by ELSD). The reaction mixture was concentrated to get crude. The crude was purified through combi-flash chromatography and compound eluted in 0-8% MeOH in DCM to get 9-fluoro-1-octylnonyl 8-{[2-(dimethylamino)ethyl][7-(9-methyldecyloxycarbonyl)heptyl]amino}octanoate (110 mg) as a yellow liquid. The compound (110 mg) was obtained after column chromatography was dissolved in dichloromethane (10 mL) and added anhydrous potassium carbonate (5 eq.) under nitrogen atmosphere and stirred vigorously for 2 h. Then, reaction mixture was filtered through hydrophobic PTFE syringe filter (25 mm×0.45 μm) using glass syringe to get 9-fluoro-1-octylnonyl 8-{[2-(dimethylamino)ethyl][7-(9-methyldecyloxycarbonyl)heptyl]amino}octanoate (55 mg, Yield=7.6%) as a colorless liquid. ¹H-NMR (400 MHz, CDCl₃-d₃): δ 4.89-4.82 (m, 1H), 4.48 (t, J=6.0 Hz, 1H), 4.37 (t, J=6.0 Hz, 1H), 4.05 (t, J=6.8 Hz, 2H), 2.57-2.51 (m, 2H), 2.42-2.33 (m, 5H), 2.30-2.25 (m, 4H), 2.23 (m, 6H), 1.74-1.69 (m, 1H), 1.63-1.57 (m, 10H), 1.57-1.49 (m, 5H), 1.44-1.38 (m, 5H), 1.30-1.25 (m, 41H), 1.54-1.21 (m, 2H), 0.89-0.85 (m, 9H). ¹⁹F-NMR (375 MHz, CDCl₃-d3): −217.99 ELSD analysis: Purity 97.51%, Calculated C₄₈H₉₆FN₂O₄, [M+H⁺]=783.73, Observed=783.50 (m/z, M+H⁺).
Example 168. Synthesis of Compound 54: 7-((8-(heptadecan-9-yloxy)-8-oxooctyl)(2-hydroxyethyl)amino)heptyl 10-methylundec-9-enoate

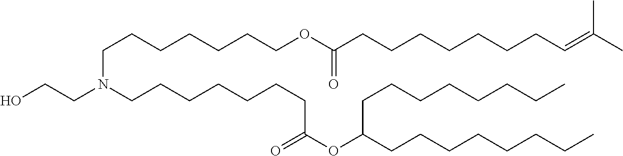 [SMILES: CCCCCCCCC(CCCCCCCC)OC(=O)CCCCCCCN(CCO)CCCCCCCOC(=O)CCCCCCCC=C(C)C]

To a stirred solution of 1-octylnonyl 8-(2-hydroxyethylamino)octanoate (0.5 g, 1.13 mmol, 1.0 eq.) in acetonitrile (10 mL), was added N-ethylbis(isopropyl)amine (439 mg. 3.4 mmol, 3 eq) and 7-bromoheptyl 10-methyl-9-undecenoate (425 mg, 1.13 mmol, 1.0 eq.). The reaction was stirred at 95° C. for 48 h. The progress of reaction was monitored by TLC and ELSD. The reaction mixture was concentrated under reduced pressure and the crude was purified by flash column chromatography (silica gel, 0-8% gradient of methanol in DCM) to get 7-{(2-hydroxyethyl)[7-(1-octylnonyloxycarbonyl)heptyl]amino}heptyl 10-methyl-9-undecenoate (150 mg) as a yellow liquid. The compound (150 mg) was obtained after column chromatography was dissolved in dichloromethane (10 mL) and added anhydrous potassium carbonate (5 eq.) under nitrogen atmosphere and stirred vigorously for 2 h. Then, reaction mixture was filtered through hydrophobic PTFE syringe filter (25 mm×0.45 μm) using glass syringe to get 7-{(2-hydroxyethyl)[7-(1-octylnonyloxycarbonyl)heptyl]amino}heptyl 10-methyl-9-undecenoate (120 mg, Yield=15%) as a yellow liquid. ¹H-NMR (400 MHz, CDCl₃-d3): δ 5.12-5.08 (m, 1H), 4.86 (quan, J=6.4 Hz, 1H), 4.05 (t, J=6.8 Hz, 2H), 3.51 (t, J=5.2 Hz, 2H), 3.04 (b, 1H), 2.56 (t, J=5.2 Hz, 2H), 2.42 (t, J=7.2 Hz, 4H), 2.30-2.25 (m, 4H), 1.95-1.92 (m, 2H), 1.68 (s, 3H), 1.64-1.55 (m, 10H), 1.50-1.38 (m, 4H), 1.42-1.37 (m, 4H), 1.36-1.25 (m, 45H), 0.87 (t, J=6.8 Hz, 6H). ELSD analysis: Purity 99.83%, Calculated C₄₆H₉₀NO₅, [M+H]=736.67, Observed=736.65 (m/z, M+H⁺).
Example 169. Synthesis of Compound 55:1-fluorohexadecan-8-yl 8-((2-hydroxyethyl)(6-((2-methylhexadecan-8-yl)oxy)-6-oxohexyl)amino)octanoate
Step 1: Synthesis of 2-methylhexadecan-8-yl 6-bromohexanoate

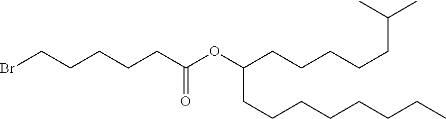 [SMILES: CCCCCCCCC(CCCCCC(C)C)OC(=O)CCCCCBr]

To a stirred solution of 6-bromohexanoic acid (3 g, 15.4 mmol, 1 eq) in dichloromethane (100 ml), was added 4-(dimethylamino)pyridin-1-ium (1.88 g, 15.4 mmol, 1 eq) and {3-[cyano(ethyl)amino]propyl}dimethylazanium chloride (5.9 g, 30.8 mmol, 3 eq) and stirred for 15 min, then 2-methylhexadecan-8-ol (3.94 g, 15.4 mmol, 1.0 eq) was added. The reaction mixture was stirred at r.t. for 48 h. The progress of reaction was monitored by TLC (SM was consumed). Water (500 mL) was added to the reaction mixture and extracted with DCM (3×500 mL). The resulting organic layer was dried over Na₂SO₄, and concentrated under reduce pressure, and the crude was purified by flash column chromatography (SiO₂: 0-2% Ethyl acetate in Hexanes) to give 1-(6-methylheptyl)nonyl 6-bromohexanoate (2.5 g. Yield=37.59%) as a pale yellow liquid. ¹H-NMR (400 MHz, CDCl3-d3): δ 4.90-483 (m, 1H), 3.54-3.38 (m, 2H), 2.28-2.38 (m, 2H), 1.90-1.76 (m, 2H), 1.67-1.62 (m, 2H), 1.55-1.48 (m, 7H), 1.27 (bs, 18H), 1.16-1.14 (m, 2H), 0.89-0.84 (m, 9H).

Step 2: Synthesis of 1-fluorohexadecan-8-yl 8-((2-hydroxyethyl)(6-((2-methylhexadecan-8-yl)oxy)-6-oxohexyl)amino)octanoate

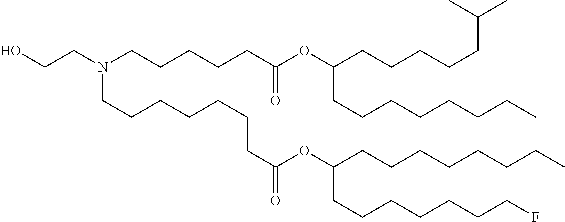 [SMILES: CCCCCCCCC(CCCCCCCF)OC(=O)CCCCCCCN(CCO)CCCCCC(=O)OC(CCCCCCCC)CCCCCC(C)C]

To a stirred solution of 1-(7-fluoroheptyl)nonyl 8-(2-hydroxyethylamino)octanoate (0.3 g, 673 μmol, 1.0 eq.) in acetonitrile (10 mL), was added N-ethylbis(isopropyl)amine (0.352 μL, 2.02 mmol, 3 eq) and 1-(6-methylheptyl)nonyl 6-bromohexanoate (292 mg, 673 μmol, 1.0 eq.). The reaction was stirred at 95° C. for 16 h. The progress of reaction was monitored by TLC and ELSD. The reaction mixture was concentrated under reduced pressure and the crude was purified by flash column chromatography (silica gel, 0-8% gradient of methanol in DCM) to get 1-(7-fluoroheptyl)nonyl 8-[(2-hydroxyethyl){5-[1-(6-methylheptyl)nonyloxycarbonyl]pentyl}amino]octanoate (120 mg) as a yellow liquid. The compound (120 mg) was obtained after column chromatography was dissolved in dichloromethane (10 mL) and added anhydrous potassium carbonate (5 eq.) under nitrogen atmosphere and stirred vigorously for 2 h. Then, reaction mixture was filtered through hydrophobic PTFE syringe filter (25 mm×0.45 μm) using glass syringe to get 1-(7-fluoroheptyl)nonyl 8-[(2-hydroxyethyl) {5-[1-(6-methylheptyl) nonyloxycarbonyl]pentyl}amino]octanoate (93 mg, Yield=17%) as a yellow liquid. ¹H-NMR (400 MHz, CDCl3-d3): δ 4.86 (quin, J=6.4 Hz, 2H), 4.48 (t, J=6.4 Hz, 1H), 4.37 (t, J=6.0 Hz, 1H), 3.51 (t, J=5.2 Hz, 2H), 2.97 (b, 1H), 2.55 (t, J=5.2 Hz, 2H), 2.45-2.40 (m, 4H), 2.30-2.25 (m, 4H), 1.72-1.59 (m, 7H), 1.52-1.38 (m, 15H), 1.42-1.25 (m, 44H), 1.17-1.14 (m, 2H), 0.89-0.87 (m, 12H). ¹⁹F-NMR (375 MHz, CDCl₃-d3): δ −218.04 ELSD analysis: Purity 99.79%, Calculated C₄₉H₉₇FNO₅, [M+H+]=798.73, Observed=798.70 (m/z, M+H+).
Example 170. Synthesis of Compound 56:1-fluoroheptadecan-9-yl 8-((2-(dimethylamino)ethyl)(8-(nonyloxy)-8-oxooctyl)amino)octanoate
Step 1: Synthesis of nonyl 8-((2-(dimethylamino)ethyl)amino)octanoate

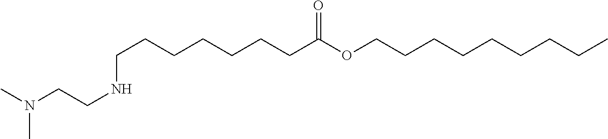 [SMILES: CCCCCCCCCOC(=O)CCCCCCCNCCN(C)C]

To a stirred solution of 2-(dimethylamino)ethanamine (2.27 g, 25.8 mmol, 1.0 eq) in acetonitrile (90 mL), was added nonyl 8-bromooctanoate (9 g, 25.8 mmol, 1.0 eq) and N-ethylbis(isopropyl)amine (13.5 mL, 77.3 mmol, 2.5 eq) to it. The reaction was stirred at 55° C. for 4 days. The progress of reaction was monitored by ELSD/TLC (64% product form in reaction mixture by ELSD). The reaction mixture was concentrated under reduced pressure and the crude was purified by flash column chromatography (SiO₂: 0-10% Methanol in Dichloromethane) to give nonyl 8-((2-(dimethylamino)ethyl)amino)octanoate (0.8 g, Yield=15.83%) as a colorless liquid. ELSD analysis: Purity 97.43%, Calculated C₂₁H₄₅N₂O₂, [M+H⁺]=357.34, Observed=357.35 (m/z, M+H⁺).
Step 2: Synthesis of 1-fluoroheptadecan-9-yl 8-((2-(dimethylamino)ethyl)(8-(nonyloxy)-8-oxooctyl)amino)octanoate

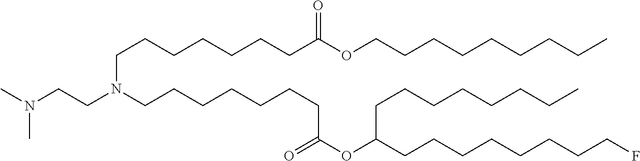 [SMILES: CCCCCCCCCOC(=O)CCCCCCCN(CCCCCCCC(=O)OC(CCCCCCCC)CCCCCCCCF)CCN(C)C]

To a stirred solution of compound nonyl 8-[2-(dimethylamino)ethylamino]octanoate (0.6 g, 1.68 mmol, 1.0 eq) in acetonitrile (10 mL), was added N-ethylbis(isopropyl)amine (0.437 μL, 2.52 mmol, 1.5 eq) and 9-fluoro-1-octylnonyl 8-bromooctanoate (807 mg, 1.68 mmol, 1.0 eq) to it. The reaction mixture was stirred at 95° C. for 48 h. The progress of reaction was monitored by TLC/ELSD (25% product formation in reaction mixture by ELSD). The reaction mixture was concentrated to get crude. The crude was purified through combi-flash chromatography and compound eluted in 0-8% MeOH in DCM. Then we performed desalting process. Compound obtained (150 mg) after flash column chromatography was dissolved in dichloromethane (10 mL) and added anhydrous potassium carbonate (5 eq.) under nitrogen atmosphere and stirred vigorously for 2 h. Then, reaction mixture was filtered through hydrophobic PTFE syringe filter (25 mm×0.45 μm) using glass syringe to get 9-fluoro-1-octylnonyl 8-{[2-(dimethylamino)ethyl][7-(nonyloxycarbonyl)heptyl]amino}octanoate (110 mg) as a colorless liquid. ¹H-NMR (400 MHz, CDCl₃-d₃): δ 4.87-4.82 (m, 1H), 4.48 (t, J=6.0 Hz, 1H), 4.37 (t, J=6.0 Hz, 1H), 4.04 (t, J=6.8 Hz, 2H), 2.54-2.51 (bs, 2H), 2.41-2.33 (m, 6H), 2.30-2.25 (m, 4H), 2.23 (m, 6H), 1.72-1.70 (m, 1H), 1.63-1.57 (m, 6H), 1.50-1.49 (m, 4H), 1.41-1.37 (m, 6H), 1.30-1.25 (m, 46H), 0.89-0.85 (m, 6H). ¹⁹F-NMR (375 MHz, CDCl₃-d3): −217.98 ELSD analysis: Purity 99.06%, Calculated C₄₆H₉₂FN₂O₄, [M+H⁺]=755.70, Observed=755.70 (m/z, M+H⁺).
Example 171. Synthesis of Compound 57:1-fluoroheptadecan-9-yl 8-((2-hydroxyethyl)(6-((7-methyloctyl)oxy)-6-oxohexyl)amino)octanoate
Step 1: Synthesis of 7-methyloctyl 6-bromohexanoate

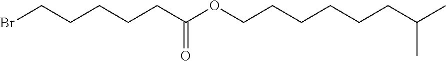 [SMILES: CC(C)CCCCCCOC(=O)CCCCCBr]

To a stirred solution of 6-bromohexanoic acid (10.8 g, 55.5 mmol, 2 eq) in dichloromethane (125 mL), was add 3-[cyano(ethyl)amino]propyl}dimethylazanium chloride (10.6 g, 55.5 mmol, 2.0 eq) and 4-(dimethylamino)pyridin-1-ium (3.39 g, 27.7 mmol, 1.0 eq) to it. The reaction was stirred for 15 min, then 7-methyl-1-octanol (4 g, 27.7 mmol, 2.0 eq) was added. The reaction mixture was stirred at r.t. for 48 h. The progress of reaction was monitored by TLC (SM was consumed). Water (100 mL) was added to the reaction mixture and extracted with DCM (3×100 mL). The resulting organic layer was dried over Na₂SO₄, and concentrated under reduce pressure, and the crude was purified by flash column chromatography (SiO₂: 0-2% Ethyl acetate in Hexanes) to get 7-methyloctyl 6-bromohexanoate (3.1 g, Yield=34.8%) as a colorless liquid. ¹H-NMR (400 MHz, CDCl₃-d3): δ 4.06 (t, J=6.8 Hz, 2H), 3.55-3.39 (m, 2H), 2.31 (t, J=7.2 Hz, 2H), 1.98-1.74 (m, 2H), 1.61-1.51 (m, 6H), 1.53-1.41 (m, 3H), 1.34-1.25 (m, 6H), 1.15-1.11 (m, 2H), 0.86 (d, J=6.4 Hz, 6H).
Step 2: Synthesis of 7-methyloctyl 6-((2-hydroxyethyl)amino)hexanoate

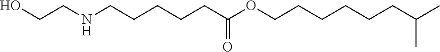 [SMILES: CC(C)CCCCCCOC(=O)CCCCCNCCO]

To a stirred solution of 2-aminoethanol (842 μL, 9.65 mmol, 1.0 eq) in acetonitrile (10 mL), was added 7-methyloctyl 6-bromohexanoate (3.1 g, 9.65 mmol, 1.0 eq) and N-ethylbis(isopropyl)amine (5.2 mL, 2.5 eq., 28.9 mmol) to it. The reaction was stirred at 55° C. for 48 h. The progress of reaction was monitored by ELSD/TLC (60% product form in reaction mixture by ELSD). The reaction mixture was concentrated under reduced pressure and the crude was purified by flash column chromatography (SiO₂: 0-10% Methanol in Dichloromethane) to give 7-methyloctyl 6-(2-hydroxyethylamino)hexanoate (980 mg, Yield=33.69%) as a brown liquid. ELSD analysis: Purity 94.84%, Calculated C₁₇H₃₆NO₃, [M+H⁺]=302.26, Observed=302.20 (m/z, M+H⁺).
Step 3: Synthesis of 1-fluoroheptadecan-9-yl 8-((2-hydroxyethyl)(6-((7-methyloctyl)oxy)-6-oxohexyl)amino)octanoate

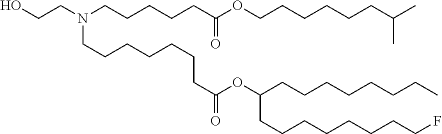 [SMILES: CCCCCCCCC(CCCCCCCCF)OC(=O)CCCCCCCN(CCO)CCCCCC(=O)OCCCCCCC(C)C]

To a stirred solution of compound 7-methyloctyl 6-(2-hydroxyethylamino)hexanoate (0.6 g, 1.99 mmol, 1.0 eq) in acetonitrile (10 mL), was added N-ethylbis(isopropyl)amine (772 mg, 5.97 mmol, 3.0 eq) and 9-fluoro-1-octylnonyl 8-bromooctanoate (954 mg, 1.99 mmol, 1.0 eq) to it. The reaction mixture was stirred at 95° C. for 48 h. The progress of reaction was monitored by TLC/ELSD (66% product formation in reaction mixture by ELSD). The reaction mixture was concentrated to get crude. The crude was purified through combi-flash chromatography and compound eluted in 0-12% MeOH in DCM. The desired fraction was collected and evaporated to get of 330 mg compound of 70% purity. The compound was submitted for reverse phase HPLC. The desired fraction were collected, evaporated and lyophilized to get 9-fluoro-1-octylnonyl 8-{(2-hydroxyethyl)[5-(7-methyloctyloxycarbonyl)pentyl]amino}octanoate (81 mg, 116 μmol) as a colorless liquid. The compound was dissolved in dichloromethane (10 ml) and filtered through Hydrophobic PTFE Syringe Filter, 25 mm*0.45 μm by glass syringe to get desired compound. 1H-NMR (400 MHz, CDCl₃-d₃): δ 4.89-4.82 (m, 1H), 4.48 (t, J=6.0 Hz, 1H), 4.37 (t, J=6.0 Hz, 1H), 4.05 (t, J=6.4 Hz, 2H), 3.51 (t, J=5.6 Hz, 2H), 2.56 (t, J=5.6 Hz, 2H), 2.45-2.40 (m, 4H), 2.28 (t, J=8.0 Hz, 4H), 1.74-1.57 (m, 10H), 1.54-1.45 (m, 6H), 1.43-1.31 (m, 4H), 1.34-1.25 (m, 33H), 1.16-1.14 (m, 2H), 0.89-0.85 (m, 9H). ¹⁹F-NMR (375 MHz, CDCl₃-d3): −217.99. ELSD analysis: Purity 99.41%, Calculated C₄₂H₈₂FNO₅, [M+H⁺]=700.62, Observed=700.60 (m/z, M+H⁺).
Example 172. Synthesis of Compound 58:1-fluoroheptadecan-9-yl 8-((2-hydroxyethyl)(8-((9-methyldec-8-en-1-yl)oxy)-8-oxooctyl)amino)octanoate
Step 1: Synthesis of 8-((tert-butyldimethylsilyl)oxy)octan-1-ol

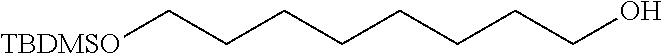 [SMILES: CC(C)(C)[Si](C)(C)OCCCCCCCCO]

To a stirred solution of 1,8-octanediol (10 g, 68.4 mmol, 1.0 eq) in dichloromethane (75 mL) was added 1H-imidazole (4.66 g, 68.4 mmol, 1.0 eq) at room temperature and stirred for 30 min. Then tert-butyl(chloro)dimethylsilane (9.28 g, 61.5 mmol, 0.9 eq) was added to the reaction mixture portion wise and the reaction mixture was stirred at room temperature for 16 h. After completion of the reaction (monitored by TLC), the reaction mixture was quenched with ice-cooled water (200 mL) and extracted with dichloromethane (3×200 mL). The organic layer was collected and dried over sodium sulphate, filtered, and concentrated under reduced pressure. The crude was purified by flash column chromatography (SiO₂: 0-10% Ethyl acetate in Hexanes), to get the product 8-[(tert-butyl)bis(methyl)siloxy]-1-octanol (7 g, Yield=39.29%) as a colorless liquid. 1H NMR (400 MHz, CDCl3-d3): δ 3.65-3.57 (m, 4H), 1.58-1.48 (m, 4H), 1.30 (b, 8H), 0.91-0.85 (m, 9H), 0.05-0.03 (m, 6H).
Step 2: 8-((tert-butyldimethylsilyl)oxy)octanal

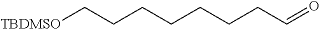 [SMILES: CC(C)(C)[Si](C)(C)OCCCCCCCC=O]

To a stirred solution of 8-[(tert-butyl)bis(methyl)siloxy]-1-octanol (10 g, 38.4 mmol 1.0 eq) in dichloromethane (135 mL) add Pyridinium chlorochromate (16.6 g, 76.8 mmol, 2 eq) at 0° C. and the reaction mixture was stirred at room temperature for 8 h. The progress of reaction was monitored by TLC (SM was consumed). The reaction mixture was diluted with pentane (200 mL) and stirred for 30 min. After 30 min, it was filtered through celite bed and wash with pentane (3×200 ml). The organic layer was collected and dried over sodium sulphate, filtered and concentrated under reduce pressure. The crude was purified by flash column chromatography (SiO₂: 0-25% Ethyl acetate in Hexanes) to give 8-[(tert-butyl)bis(methyl)siloxy]octanal (7 g, Yield=70.5%) as colorless liquid. ¹H-NMR (400 MHz, CDCl₃-d3): δ 9.76 (t, J=1.6 Hz, 1H), 3.64-3.57 (m, 2H), 2.43-2.32 (m, 2H), 1.63-1.60 (m, 2H), 1.52-1.47 (m, 2H), 1.32 (b, 6H), 0.92-0.85 (m, 9H), 0.04 (b, 6H).
Step 3: Synthesis of tert-butyldimethyl((9-methyldec-8-en-1-yl)oxy)silane

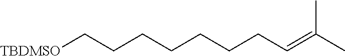 [SMILES: CC(C)=CCCCCCCCO[Si](C)(C)C(C)(C)C]

To a stirred solution of isopropyltriphenylphosphonium bromide (10.4 g, 27.1 mmol, 1.0 eq) in tetrahydrofuran (54 mL) was added n-Butyllithium (2.6 g, 40.6 mmol, 1.5 eq) dropwise at −78° C. The reaction mixture was stirred at room temperature for 1 hr, then the mixture was cooled again to −78° C. and 8-[(tert-butyl)bis(methyl)siloxy]octanal (7 g, 27.1 mmol, 1.0 eq) (dissolved in THF) was added dropwise. The reaction mixture was stirred at room temperature for 16 h. After completion of reaction (monitored by TLC), the reaction mixture was quenched with aq. ammonium chloride solution and extracted with ethyl acetate (3×200 mL). The organic layer was collected and dried over sodium sulphate, filtered and concentrated under vacuum to get the crude. The crude product was purified through flash column chromatography (SiO₂: 0-3% Ethyl acetate in Hexane), to get (tert-butyl)bis(methyl)(9-methyl-8-decenyloxy)silane (6.3 g, Yield=81.75%) as a colorless liquid. ¹H-NMR (400 MHz, CDCl₃-d3): δ 5.11 (t, J=6.8 Hz, 1H), 3.59 (t, J=6.4 Hz, 2H), 1.96-1.93 (m, 2H), 1.68 (s, 3H), 1.59 (s, 3H), 1.56-1.47 (m, 2H), 1.28 (s, 8H), 0.89 (s, 9H), 0.04 (m, 6H).
Step 4: Synthesis of 9-methyldec-8-en-1-ol

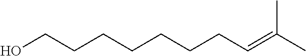

To a stirred solution of tert-butyl)bis(methyl)(9-methyl-8-decenyloxy)silane (6 g, 21.1 mmol) in tetrahydrofuran (50 mL), pyridine hydrofluoride (10.4 g, 105 mmol, 5 eq) was added at 0° C. The resulting reaction mixture was stirred for 16 h at room temperature. The progress of reaction mass was monitored by ELSD/TLC (SM was consumed). The resulting reaction mixture was quenched with cold aqueous sodium bicarbonate solution up to pH 8, and extract with ethyl acetate (3×100 mL). The resulting organic layer was dried over Na₂SO₄, concentrated under reduce pressure, and the crude was purified by flash column chromatography (SiO₂: 0-50% ethyl acetate in hexane), to obtain the desired produced 9-methyl-8-decen-1-ol (3.3 g, 91.9% yield) as light yellow liquid. ¹H NMR (400 MHz, CDCl₃-d3): δ 5.14-5.10 (m, 1H), 3.65 (t, J=6.4 Hz, 2H), 1.98-1.95 (m, 2H), 1.70 (s, 3H), 1.61 (s, 3H), 1.42 (m, 2H), 1.32 (m, 8H).
Step 5: Synthesis of 9-methyldec-8-en-1-yl 8-bromooctanoate

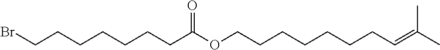 [SMILES: CC(C)=CCCCCCCCOC(=O)CCCCCCCBr]

To a stirred solution of 8-bromooctanoic acid (10.5 g, 47 mmol, 2 eq.,) in dichloromethane (125 mL), was add 3-[cyano(ethyl)amino]propyl}dimethylazanium chloride (9.01 g, 47 mmol, 2.0 eq) and 4-(dimethylamino)pyridin-1-ium (2.87 g, 23.5 mmol, 1.0 eq) to it. The reaction was stirred for 15 min, then 9-methyl-8-decen-1-ol (4 g, 23.5 mmol, 1 eq) was added. The reaction mixture was stirred at room temperature for 48 h. The progress of reaction was monitored by TLC (SM was consumed). Water (100 mL) was added to the reaction mixture and extracted with DCM (3×100 mL). The resulting organic layer was dried over Na₂SO₄, and concentrated under reduce pressure, and the crude was purified by flash column chromatography (SiO₂: 0-10% Ethyl acetate in Hexanes) to get 9-methyl-8-decenyl 8-bromooctanoate (3.9 g, Yield=44.23%) as a colorless liquid. ¹H-NMR (400 MHz, CDCl₃-d3): δ 5.12-5.08 (m, 1H), 4.05 (t, J=6.8 Hz, 1H), 3.39 (t, J=6.8 Hz, 1H), 2.29 (t, J=7.2 Hz, 2H), 1.91-1.92 (m, 2H), 1.88-1.81 (m, 2H), 1.69 (s, 3H), 1.68 (m, 7H), 1.44-1.39 (m, 2H), 1.34-1.30 (m, 13H).
Step 6: Synthesis of 9-methyldec-8-en-1-yl 8-((2-hydroxyethyl)amino)octanoate

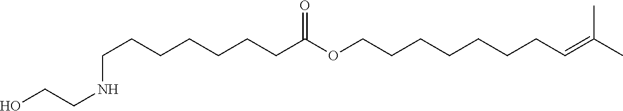 [SMILES: CC(C)=CCCCCCCCOC(=O)CCCCCCCNCCO]

To a stirred solution of 2-aminoethanol (628 μL, 10.4 mmol, 1.0 eq) in acetonitrile (50 mL), was added 9-methyl-8-decenyl 8-bromooctanoate (3.9 g, 10.4 mmol, 1.0 eq) and N-ethylbis(isopropyl)amine (4.62 mL, 26 mmol, 2.5 eq) to it. The reaction was stirred at 55° C. for 48 h. The progress of reaction was monitored by ELSD/TLC (53% product form in reaction mixture by ELSD). The reaction mixture was concentrated under reduced pressure and the crude was purified by flash column chromatography (SiO₂: 0-10% Methanol in Dichloromethane) to give 9-methyl-8-decenyl 8-(2-hydroxyethylamino)octanoate (0.8 g, Yield=21.66%) as a colorless liquid. ELSD analysis: Purity 99.07%, Calculated C₂₁H₄₂NO₃, [M+H]=356.31, Observed=356.30 (m/z, M+H⁺).
Step 7: Synthesis of 1-fluoroheptadecan-9-yl 8-((2-hydroxyethyl)(8-((9-methyldec-8-en-1-yl)oxy)-8-oxooctyl)amino)octanoate

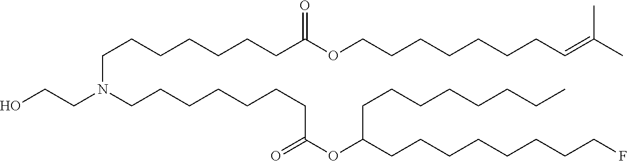 [SMILES: CCCCCCCCC(CCCCCCCCF)OC(=O)CCCCCCCN(CCO)CCCCCCCC(=O)OCCCCCCCC=C(C)C]

To a stirred solution of compound 9-methyl-8-decenyl 8-(2-hydroxyethylamino)octanoate (0.3 g, 844 μmol, 1.0 eq) in acetonitrile (10 mL), was added N-ethylbis(isopropyl)amine (451 μL, 2.53 mmol, 3 eq) and 9-fluoro-1-octylnonyl 8-bromooctanoate (405 mg, 844 μmol, 1.0 eq) to it. The reaction mixture was stirred at 95° C. for 48 h. The progress of reaction was monitored by TLC/ELSD (73% product formation in reaction mixture by ELSD). The reaction mixture was concentrated then quenched with saturated sodium bicarbonate solution (20 ml) and extracted with dichloromethane (20 mL×3). The resulting organic layer was dried over sodium sulphate, and concentrated under reduce pressure, and the crude was purified by flash column chromatography (SiO₂: 0-6% methanol in dichloromethane), to give 9-fluoro-1-octylnonyl 8-{(2-hydroxyethyl)[7-(9-methyl-8-decenyloxycarbonyl)heptyl]amino}octanoate (190 mg, Yield=29.39%) as a pale yellow liquid. The compound was dissolved in dichloromethane (10 ml) and filtered through Hydrophobic PTFE Syringe Filter, 25 mm*0.45 μm by glass syringe to get desired compound. ¹H-NMR (400 MHz, CDCl₃-d3): δ 5.10 (t, J=7.2 Hz, 1H), 4.89-4.82 (m, 1H), 4.48 (t, J=6.0 Hz, 1H), 4.37 (t, J=6.0 Hz, 1H), 4.05 (t, J=6.8 Hz, 2H), 3.57 (bs, 2H), 2.69-2.41 (bs, 6H), 2.30-2.25 (m, 4H), 1.96-1.93 (m, 2H), 1.79-1.61 (m, 10H), 1.50-1.49 (m, 8H), 1.30-1.28 (m, 46H), 0.89-0.85 (m, 3H). ¹⁹F-NMR (375 MHz, CDCl₃-d₃): −217.99 ELSD analysis: Purity 99.75%, Calculated C₄₆H₈₉FNO₅, [M+H]=754.66, Observed=754.65 (m/z, M+H⁺).
Example 173. Synthesis of Compound 59:9-methyldecyl 8-((8-(heptadecan-9-yloxy)-8-oxooctyl)(2-hydroxyethyl)amino)-2-methyloctanoate
Step 1: Synthesis of 9-methyldecyl 8-bromo-2-methyloctanoate

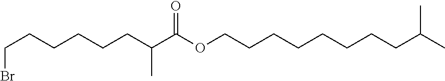 [SMILES: CC(C)CCCCCCCCOC(=O)C(C)CCCCCCBr]

To a stirred solution of 8-bromo-2-methyloctanoic acid (4.4 g, 18.6 mmol, 1 eq) in dichloromethane (80 ml), was added 4-(dimethylamino)pyridin-1-ium (3.4 g, 27.8 mmol, 1.5 eq) and {3-[cyano(ethyl)amino]propyl}dimethylazanium chloride (10.7 g, 55.7 mmol, 3 eq). The reaction mixture was stirred for 15 min, then added 9-methyl-1-decanol (3.2 g, 18.6 mmol, 1.0 eq). The reaction mixture was stirred at r.t. for 6 h. The progress of reaction was monitored by TLC (SM was consumed). Water (500 ml) was added to the reaction mixture, and extracted with DCM (3×500 ml). The resulting organic layer was dried over sodium sulphate, and concentrated under reduce pressure, and the crude was purified by flash column chromatography (SiO₂: 0-20% Ethyl acetate in Hexanes), to get a 9-methyldecyl 8-bromo-2-methyloctanoate (4.2 g, Yield=57.83%) as a colorless liquid. ¹H-NMR (400 MHz, CDCl₃-d₃): δ 4.05 (t, J=6.8 Hz, 2H), 3.52 (t, J=6.4 Hz, 2H), 2.45-2.37 (m, 1H), 1.79-1.72 (m, 2H), 1.68-1.53 (m, 4H), 1.45-1.39 (m, 3H), 1.45-1.20 (m, 14H), 1.19-1.12 (m, 5H), 0.86 (d, J=6.4 Hz, 6H).
Step 2: Synthesis of 9-methyldecyl 8-((8-(heptadecan-9-yloxy)-8-oxooctyl)(2-hydroxyethyl)amino)-2-methyloctanoate

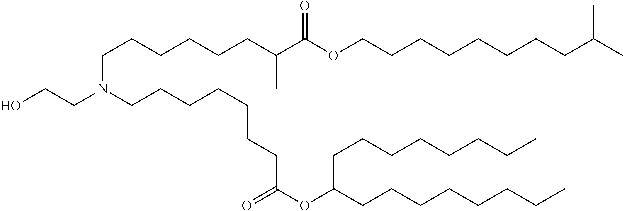 [SMILES: CCCCCCCCC(CCCCCCCC)OC(=O)CCCCCCCN(CCO)CCCCCCC(C)C(=O)OCCCCCCCCC(C)C]

To a stirred solution of compound 1-octylnonyl 8-(2-hydroxyethylamino)octanoate (0.4 g, 906 μmol, 1.0 eq) in acetonitrile (10 mL) was added N-ethylbis(isopropyl)amine (474 μL, 2.72 mmol, 3 eq) and 9-methyldecyl 8-bromo-2-methyloctanoate (354 mg, 906 μmol, 1.0 eq). The reaction mixture was stirred at 100° C. for 2 days. The progress of reaction was monitored by TLC/ELSD. The reaction mixture was concentrated then quenched with saturated sodium bicarbonate solution (50 ml) and extracted with dichloromethane (50 mL×3). The resulting organic layer was dried over sodium sulphate, and concentrated under reduce pressure. The crude was purified by flash column chromatography (SiO₂: 0-8% methanol in dichloromethane), to get 9-methyldecyl 8-{(2-hydroxyethyl)[7-(1-octylnonyloxycarbonyl)heptyl]amino}-2-methyloctanoate (200 mg Yield=29.35%) as a pale yellow liquid. The compound was dissolved in dichloromethane (10 ml) and filtered through Hydrophobic PTFE Syringe Filter, 25 mm*0.45 μm by glass syringe to get desired compound. 1H-NMR (400 MHz, CDCl₃-d3): δ 4.88-4.82 (m, 1H), 4.05 (t, J=6.8 Hz, 2H), 3.51 (t, J=5.2 Hz, 2H), 2.55 (t, J=5.2 Hz, 2H), 2.42 (t, J=7.6 Hz, 4H), 2.27 (t, J=7.6 Hz, 2H), 1.65-1.57 (m, 6H), 1.52-1.47 (m, 6H), 1.45-1.38 (m, 4H), 1.30-1.23 (m, 46H), 1.16-1.12 (m, 5H), 0.89-0.85 (m, 12H). ELSD analysis: Purity 99.69%, Calculated C₄₇H₉₄NO₅, [M+H]=752.71, Observed=752.70 (m/z, M+H+).
Example 174. Synthesis of Compound 60: 7-((7-((10-fluoro-2-octyldecanoyl)oxy)heptyl)(2-hydroxyethyl)amino)heptyl 10-methylundecanoate
Step 1: Synthesis of 9-hydroxynonyl acetate

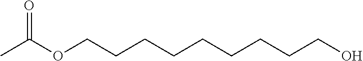

To a stirred solution of 1,9-nonanediol (150 g, 936 mmol, 1.0 eq.) in tetrahydrofuran (1.24 L), pyridine (151 mL, 1.87 mol, 2 eq.) and acetyl acetate (91.9 mL, 936 mmol, 1.0 eq) was added at 0° C. and stirred at 25° C. for 16 h. The reaction mixture was quenched with aqueous sodium bicarbonate solution (800 mL) and extracted with ethyl acetate (5×500 mL). The organic layer was dried over sodium sulphate, filtered and concentrated under reduced pressure. The crude was purified by flash column chromatography (silica gel, 0-50% gradient of ethyl acetate in hexane) to get 9-hydroxynonyl acetate (80 g, Yield=42.25%) as a colorless liquid. ¹H NMR (400 MHz, CDCl₃-d3): δ 4.04 (t, J=6.8 Hz, 2H), 3.63 (t, J=6.8 Hz, 2H), 2.04 (s, 3H), 1.65-1.51 (m, 4H), 1.41-1.25 (m, 10H).
Step 2: Synthesis of 9-fluorononyl acetate

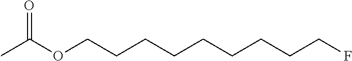

To a stirred solution of 9-hydroxynonyl acetate (25 g, 124 mmol, 1.0 eq.) in dichloromethane (250 mL) was cooled to −78° C. and N,N-diethyl(trifluorothio)amine (24.9 mL, 185 mmol, 1.5 eq.) was added dropwise. Reaction mixtures was stirred at 25° C. for 16 h. Then reaction mass cooled to −78° C. and added dropwise aqueous sodium bicarbonate solution (100 mL) and extracted with ethyl acetate (3×250 mL). The organic layer was collected dried over sodium sulphate, filtered and evaporated under reduced pressure. The crude was purified by flash column chromatography (silica gel, 0-30% gradient of ethyl acetate in hexane) to obtain 9-fluorononyl acetate (23 g, Yield=91.1%) as colorless liquid. ¹H NMR (400 MHz, CDCl₃-d3): δ 4.49 (t, J=6.0 Hz, 1H), 3.37 (t, J=6.0 Hz, 1H), 4.05 (t, J=6.8 Hz, 2H), 2.04 (s, 3H), 1.78-1.58 (m, 4H), 1.45-1.28 (m, 10H).
Step 3: Synthesis of 9-fluorononan-1-ol

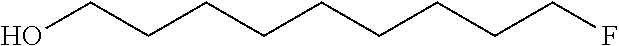 [SMILES: OCCCCCCCCCF]

To a stirred solution of 9-fluorononyl acetate (20 g, 97.9 mmol, 1.0 eq.) in tetrahydrofuran (125 mL) was cooled to −78° C. and Lithium aluminium hydride (7.43 g, 196 mmol, 2 eq.) was added dropwise. After complete addition, reaction mixture was stirred at 25° C. for 16 h. TLC showed formation of new spots and starting material was consumed. The reaction mass was cooled to −10° C. and quenched with aqueous ammonium chloride (100 mL). Then, reaction mass was filtered through celite bed. The filtrate was collected and evaporated to dryness. The crude was purified by flash column chromatography (silica gel, 0-12% gradient of ethyl acetate in hexane) to obtain 9-fluoro-1-nonanol (15 g, Yield=94.43%) as a colorless liquid. ¹H NMR (400 MHz, CDCl₃-d3): δ 4.49 (t, J=6.0 Hz, 1H), 4.37 (t, J=6.0 Hz, 1H), 3.63 (t, J=6.8 Hz, 2H), 1.75-1.61 (m, 2H), 1.58-1.51 (m, 2H), 1.45-1.28 (m, 10H).
Step 4: Synthesis of 9-fluorononanoic acid

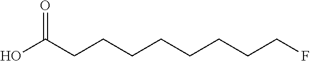 [SMILES: O=C(O)CCCCCCCCF]

A mixture of chromamethanetrione-sulfuric acid (1/1)(32.9 mL, 216 mmol, 5.0 eq.) and water (14 mL) was cooled to −10° C. Then, 9-fluoro-1-nonanol (7 g. 43.1 mmol, 1.0 eq.) dissolved in acetone (70 mL) was added dropwise. The reaction mass was stirred at room temperature for 15 h. After complete consumption of starting materials (monitored by TLC), reaction mixture was dilute with water (100 mL) and extracted with diethyl ether (5×100 mL). The resulting organic layer was dried over sodium sulfate and concentrated under reduce pressure. The crude was purified by flash column chromatography (silica gel, 0-25% gradient of ethyl acetate in hexane) to obtain 9-fluorononanoic acid (4.2 g, Yield=55.24%) as a white crystal. ¹H NMR (400 MHz, CDCl₃-d3): δ 11.50-10.01 (bs, 1H), 4.49 (t, J=6.4 Hz, 1H), 4.37 (t, J=6.4 Hz, 1H), 2.35 (t, J=7.2 Hz, 2H), 1.75-1.61 (m, 4H), 1.45-1.28 (m, 8H).
Step 5: Synthesis of 10-fluoro-2-octyldecanoic acid

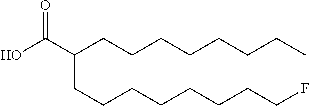 [SMILES: CCCCCCCCC(CCCCCCCCF)C(=O)O]

To a stirred solution of 9-fluorononanoic acid (2.5 g, 12.7 mmol, 1.0 eq.) in tetrahydrofuran (30.3 mL), was added sodium hydride (611 mg, 15.3 mmol, 1.2 eq.) at 0° C. and stirred for 30 minutes. Then, lithium bis(isopropyl)azanide (1.64 g, 15.3 mmol, 1.2 eq.) was added to the reaction mixture at the −50° C. The reaction mixture was stirred at room temperature for 30 minutes. Then, 1-iodooctane (3.06 g. 12.7 mmol, 1.0 eq.) was added and reaction mixture was stirred at 45° C. for 16 h. The progress of reaction was monitored by TLC. The reaction mixture was poured into 1M HCl (250 mL) and extracted with ethyl acetate (3×250 mL). The resulting organic layer was dried over sodium sulfate, and concentrated under reduce pressure. The crude thus obtained was purified by flash column chromatography (silica gel, 0-2% gradient of ethyl acetate in hexane) to obtain 2-(7-fluoroheptyl)decanoic acid (0.8 g, Yield=27.77%) as a yellow liquid. ¹H NMR (400 MHz, CDCl₃-d3): δ 4.49 (t, J=6.4 Hz, 1H), 4.37 (t, J=6.4 Hz, 1H), 2.41-2.28 (m, 1H), 1.75-1.52 (m, 4H), 1.51-1.40 (m, 2H), 1.37-1.21 (m, 20H), 0.89-0.85 (m, 3H).
Step 6: Synthesis of 7-bromoheptyl 10-fluoro-2-octyldecanoate

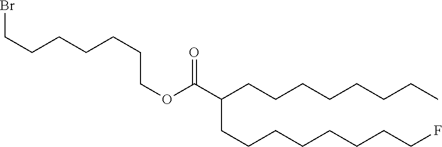 [SMILES: CCCCCCCCC(CCCCCCCCF)C(=O)OCCCCCCCBr]

To a stirred solution of 2-(7-fluoroheptyl)decanoic acid (596 mg, 2.07 mmol, 1.0 eq) in dichloromethane (20 mL), was added 1-Ethyl-3-(3-dimethylaminopropyl) carbodiimide (1.19 g, 6.2 mmol, 3 eq.) and N,N-dimethyl-4-pyridylamine (252 mg, 2.07 mmol, 1.0 eq.). Reaction mixture was stirred for 10 minutes then 7-bromo-1-heptanol (403 mg, 2.07 mmol, 1.0 eq.) was added. The reaction mixture was stirred at room temperature for 16 h. TLC showed formation of new spots and starting material was consumed. Reaction mixture was quenched with brine solution (50 mL) and extracted with dichloromethane (2×100 mL). The organic layer was dried over sodium sulphate, filtered and concentrated under reduced pressure. The crude was purified by flash column chromatography (silica gel, 0-2% gradient of ethyl acetate in hexane) to obtain 7-bromoheptyl 2-(7-fluoroheptyl) decanoate (560 mg, Yield=58.22%) as a colorless liquid. ¹H NMR (400 MHz, CDCl₃-d3): δ 4.51 (t, J=6.0 Hz, 1H), 4.39 (t, J=6.4 Hz, 1H), 4.09 (t, J=6.4 Hz, 2H), 3.43 (t, J=6.4 Hz, 2H), 2.36-2.31 (m, 1H), 1.92-1.82 (quin, J=7.2 Hz, 2H), 1.78-1.58 (m, 4H), 1.51-1.20 (m, 30H), 0.90 (t, J=6.8 Hz, 3H).
Step 7: Synthesis of 7-((7-((10-fluoro-2-octyldecanoyl)oxy)heptyl)(2-hydroxyethyl)amino)heptyl 10-methylundecanoate

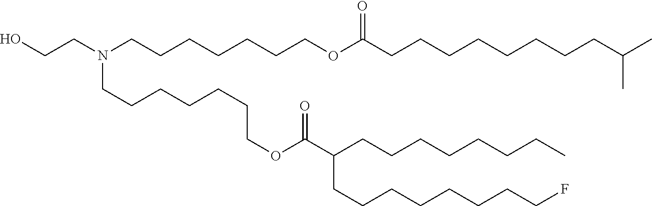 [SMILES: CCCCCCCCC(CCCCCCCCF)C(=O)OCCCCCCCN(CCO)CCCCCCCOC(=O)CCCCCCCCC(C)C]

To a stirred solution of 7-bromoheptyl 10-fluoro-2-octyldecanoate (443 mg, 923 μmol, 1.0 eq) in acetonitrile (10 mL) was added 7-(2-hydroxyethylamino)heptyl 10-methylundecanoate (330 mg, 923 μmol, 1.0 eq) and N-ethylbis(isopropyl)amine (484 μL, 2.77 mmol, 3 eq). The reaction was stirred at 100° C. for 16 h. The progress of reaction was monitored by ELSD/TLC (81% product form in reaction mixture by ELSD). The reaction mixture was concentrated under reduced pressure and the crude was purified by flash column chromatography (SiO₂: 0-10% Methanol in Dichloromethane). The desired fraction was collected and evaporated to get 280 mg compound of 93.99% purity. The compound was purified by reverse phase HPLC. The desired fraction were collected, evaporated and lyophilized to get 7-{[7-(9-fluoro-1-octylnonylcarbonyloxy)heptyl](2-hydroxyethyl)amino}heptyl 10-methylundecanoate (104 mg. 14.9%) as a colorless liquid. The compound was dissolved in dichloromethane (10 ml) and filtered through Hydrophobic PTFE Syringe Filter, 25 mm*0.45 μm by glass syringe to get desired compound. ¹H-NMR (400 MHz, CDCl₃-d₃): δ 4.48 (t, J=6.0 Hz, 1H), 4.36 (t, J=6.0 Hz, 1H), 4.07-4.03 (m, 4H), 3.54 (m, 2H), 2.60-2.39 (t, J=7.6 Hz, 6H), 2.33-2.26 (m, 3H), 1.70-1.67 (m, 1H), 1.62-1.59 (m, 8H), 1.55-1.39 (m, 8H), 1.39-1.24 (m, 44H), 1.16-1.11 (m, 2H), 0.89-0.84 (m, 9H). ¹⁹F-NMR (375 MHz, CDCl₃-d3): δ −217.99. ELSD analysis: Purity 99.58%, Calculated C₄₆H₉₁FNO₅, [M+H]=756.68, Observed=756.65 (m/z, M+H⁺).
Example 175. Synthesis of Compound 61: nonyl 8-((8-((1-fluoroheptadecan-9-yl)oxy)-8-oxooctyl)(2-hydroxyethyl)amino)-2-methyloctanoate
Step 1: Synthesis of 1-fluoroheptadecan-9-yl 8-((2-hydroxyethyl)amino)octanoate

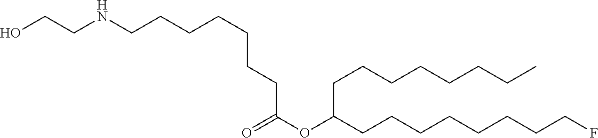 [SMILES: CCCCCCCCC(CCCCCCCCF)OC(=O)CCCCCCCNCCO]

(1.6 g, 3.34 mmol, 1.0 eq) in acetonitrile (15 mL) was added 2-aminoethanol (204 mg, 3.34 mmol, 1.0 eq) and N-ethylbis(isopropyl)amine (699 μL, 4.0 mmol, 1.2 eq). The reaction was stirred at 70° C. for 2 days. The progress of reaction was monitored by ELSD/TLC (SM was consumed). The reaction mixture was concentrated under reduced pressure and the crude was purified by flash column chromatography (SiO₂: 0-10% Methanol in Dichloromethane) to get 9-fluoro-1-octylnonyl 8-(2-hydroxyethylamino)octanoate (970 mg, Yield=63.24%) as a white gummy solid. ELSD analysis: Purity 98.90%, Calculated C₂₇H₅₅FNO₃, [M+H]=460.41, Observed=460.30 (m/z, M+H⁺).
Step 2: Synthesis of nonyl 8-((8-((1-fluoroheptadecan-9-yl)oxy)-8-oxooctyl)(2-hydroxyethyl)amino)-2-methyloctanoate

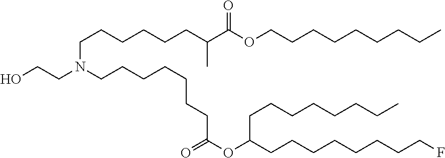 [SMILES: CCCCCCCCCOC(=O)C(C)CCCCCCN(CCO)CCCCCCCC(=O)OC(CCCCCCCC)CCCCCCCCF]

To a stirred solution of 9-fluoro-1-octylnonyl 8-(2-hydroxyethylamino)octanoate (506 mg, 1.1 mmol, 1.0 eq) in acetonitrile (10 mL) was added nonyl 8-bromo-2-methyloctanoate (0.4 g, 1.1 mmol, 1.0 eq) and N-ethylbis(isopropyl)amine (577 μL, 3.3 mmol, 3 eq). The reaction was stirred at 100° C. for 48 h. The progress of reaction was monitored by ELSD/TLC (81% product form in reaction mixture by ELSD). The reaction mixture was concentrated under reduced pressure and the crude was purified by flash column chromatography (SiO₂: 0-10% Methanol in Dichloromethane) to get nonyl 8-{[7-(9-fluoro-1-octylnonyloxycarbonyl)heptyl](2-hydroxyethyl)amino}-2-methyloctanoate (160 mg) as colorless liquid. The compound (160 mg) was obtained after column chromatography was dissolved in dichloromethane (10 mL) and added anhydrous potassium carbonate (5 eq) under nitrogen atmosphere and stirred vigorously for 2 h. Then, reaction mixture was filtered through hydrophobic PTFE syringe filter (25 mm×0.45 μm) using glass syringe to get nonyl 8-{[7-(9-fluoro-1-octylnonyloxycarbonyl)heptyl](2-hydroxyethyl)amino}-2-methyloctanoate (135 mg. Yield=16.5%) as a colorless liquid. ¹H-NMR (400 MHz, CDCl₃-d3): δ 4.88-4.82 (m, 1H), 4.48 (t, J=6.0 Hz, 1H), 4.37 (t, J=6.0 Hz, 1H), 4.05 (t, J=6.8 Hz, 2H), 3.51 (t, J=5.2 Hz, 2H), 3.00 (b, 1H), 2.55 (t, J=5.6 Hz, 2H), 2.43-2.38 (m, 5H), 2.27 (t, J=7.6 Hz, 2H), 1.72-1.59 (m, 8H), 1.54-1.47 (m, 4H), 1.46-1.36 (m, 7H), 1.35-1.25 (m, 42H), 1.13 (d, J=6.8 Hz, 3H), 0.89-0.85 (m, 6H). ¹⁹F-NMR (375 MHz, CDCl₃-d₃): −217.99. ELSD analysis: Purity 99.88%, Calculated C₄₅H₈₉FNO₅, [M+H]=742.66, Observed=742.65 (m/z, M+H⁺).
Example 176. Synthesis of Compound 62: nonyl 8-((8-(heptadecan-9-yloxy)-8-oxooctyl)(4-hydroxybutyl)amino)-2-methyloctanoate
Step 1: Synthesis of heptadecan-9-yl 8-((4-hydroxybutyl)amino)octanoate

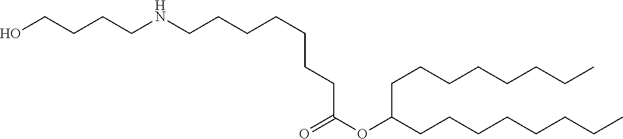 [SMILES: CCCCCCCCC(CCCCCCCC)OC(=O)CCCCCCCNCCCCO]

To a stirred solution of 4-amino-1-butanol (966 mg, 10.8 mmol, 1.0 eq) in acetonitrile (50 mL), was added heptadecan-9-yl 8-bromooctanoate (5 g, 10.8 mmol, 1.0 eq) and N-ethylbis(isopropyl)amine (4.73 mL, 2.5 eq., 27.1 mmol) to it. The reaction was stirred at 55° C. for 48 h. The progress of reaction was monitored by ELSD/TLC (56% product form in reaction mixture by ELSD). The reaction mixture was concentrated under reduced pressure and the crude was purified by flash column chromatography (SiO₂: 0-10% Methanol in Dichloromethane) to give 1-octylnonyl 8-(4-hydroxybutylamino)octanoate (2.4 g, Yield=47.16%) as a colorless liquid. ELSD analysis: Purity 99.07%, Calculated C₂₉H₆₀NO₃, [M+H]=470.46, Observed=470.35 (m/z, M+H⁺).
Step 2: nonyl 8-((8-(heptadecan-9-yloxy)-8-oxooctyl)(4-hydroxybutyl)amino)-2-methyloctanoate

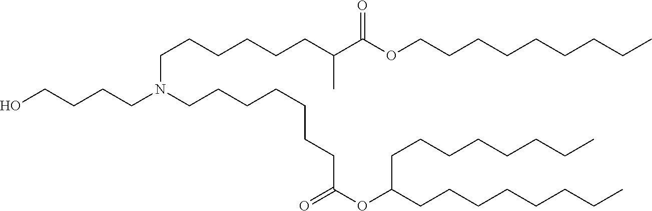 [SMILES: CCCCCCCCCOC(=O)C(C)CCCCCCN(CCCCO)CCCCCCCC(=O)OC(CCCCCCCC)CCCCCCCC]

To a stirred solution of compound nonyl 8-bromo-2-methyloctanoate (0.4 g, 1.1 mmol, 1.0 eq) in acetonitrile (10 mL), was added N-ethylbis(isopropyl)amine (577 μL, 3.3 mmol, 3 eq) and 1-octylnonyl 8-(4-hydroxybutylamino)octanoate (517 mg, 1.1 mmol, 1.0 eq) to it. The reaction mixture was stirred at 95° C. for 72 h. The progress of reaction was monitored by TLC/ELSD (60% product formation in reaction mixture by ELSD). The reaction mixture was concentrated and quenched with saturated sodium bicarbonate solution (100 ml) and extracted with dichloromethane (100 mL×3). The resulting organic layer was dried over sodium sulphate, and concentrated under reduce pressure, and the crude was purified by flash column chromatography (SiO₂: 0-6% methanol in dichloromethane), to give nonyl 8-((8-(heptadecan-9-yloxy)-8-oxooctyl)(4-hydroxybutyl)amino)-2-methyloctanoate (225 mg, Yield=27.17%). The compound was dissolved in dichloromethane (10 ml) and filtered through Hydrophobic PTFE Syringe Filter, 25 mm*0.45 μm by glass syringe to get desired compound. ¹H-NMR (400 MHz, CDCl₃-d₃): δ 4.89-4.82 (m, 1H), 4.05 (t, J=6.8 Hz, 2H), 3.54 (bs, 2H), 2.43-2.37 (bs, 7H), 2.27 (t, J=7.6 Hz, 2H), 1.64-1.59 (m, 8H), 1.50-1.46 (m, 10H), 1.30-1.25 (m, 48H), 1.13 (d, J=6.8 Hz, 3H), 0.87 (t, J=6.8 Hz, 9H). ELSD analysis: Purity 99.91%, Calculated C₄₇H₉₄NO₅, [M+H⁺]=752.71, Observed=752.70 (m/z, M+H⁺).
Example 177. Synthesis of Compound 63:1-fluoroheptadecan-9-yl 6-((4-hydroxybutyl)(6-((11-methyldodecyl)oxy)-6-oxohexyl)amino)hexanoate
Step 1: Synthesis of 1-fluoroheptadecan-9-yl 6-bromohexanoate

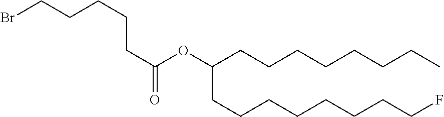 [SMILES: CCCCCCCCC(CCCCCCCCF)OC(=O)CCCCCBr]

To a stirred solution of 6-bromohexanoic acid (2.0 g, 10.2 mmol, 1.5 eq) in dichloromethane (25 mL), was added 4-(dimethylamino)pyridin-1-ium (1.25 g, 10.2 mmol, 1.5 eq) and {3-[cyano(ethyl)amino]propyl}dimethylazanium chloride (3.27 g, 17.1 mmol, 2.5 eq). The reaction mixture was stirred for 15 min and added 1-fluoro-9-heptadecanol (1.5 g, 0.8 eq., 5.47 mmol) to it. The reaction mixture was stirred at room temperature (r.t.) for 48 hours (h). The progress of reaction was monitored by TLC (SM was consumed). Water (250 mL) was added to the reaction mixture, and extracted with DCM (3×250 mL). The resulting organic layer was dried over Na₂SO₄, and concentrated under reduce pressure, and the crude was purified by flash column chromatography (SiO₂: 0-2% Ethyl acetate in Hexanes) to give 9-fluoro-1-octylnonyl 6-bromohexanoate (1.6 g, Yield=51.8%) as a pale yellow liquid. ¹H NMR (400 MHz, CDCl₃-d3): δ 4.90-4.83 (m, 1H), 4.49 (t, J=6.0 Hz, 1H), 4.37 (t, J=6.0 Hz, 1H), 3.55-3.38 (m, 2H), 2.30 (t, J=7.6 Hz, 2H), 1.91-1.76 (m, 2H), 1.75-1.62 (m, 4H), 1.52-1.45 (m, 6H), 1.44-1.26 (m, 22H), 0.89-0.86 (t, J=6.8 Hz, 3H).
Step 2: Synthesis of 11-methyldodecyl 6-((4-hydroxybutyl)amino)hexanoate

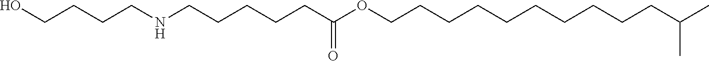 [SMILES: CC(C)CCCCCCCCCCOC(=O)CCCCCNCCCCO]

To a stirred solution of 4-amino-1-butanol (236 mg, 2.65 mmol, 1.0 eq) in acetonitrile (50 mL), was added 11-methyldodecyl 6-bromohexanoate (1 g, 2.65 mmol, 1.0 eq) and N-ethylbis(isopropyl)amine (1.16 mL, 6.62 mmol, 2.5 eq) to it. The reaction was stirred at 55° C. for 48 h. The progress of reaction was monitored by ELSD/TLC (56% product form in reaction mixture by ELSD). The reaction mixture was concentrated under reduced pressure and the crude was purified by flash column chromatography (SiO₂: 0-10% Methanol in Dichloromethane) to give 11-methyldodecyl 6-(4-hydroxybutylamino)hexanoate (550 mg, Yield=53.83%) as a colorless liquid. ELSD analysis: Purity 98.29%, Calculated C₂₃H₄₈NO₃, [M+H⁺]=386.36, Observed=386.30 (m/z, M+H⁺).
Step 3: Synthesis of 1-fluoroheptadecan-9-yl 6-((4-hydroxybutyl)(6-((11-methyldodecyl)oxy)-6-oxohexyl)amino)hexanoate

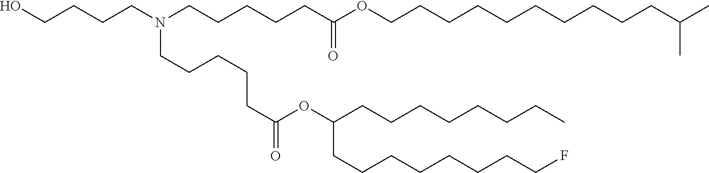 [SMILES: CCCCCCCCC(CCCCCCCCF)OC(=O)CCCCCN(CCCCO)CCCCCC(=O)OCCCCCCCCCCC(C)C]

To a stirred solution of compound 9-fluoro-1-octylnonyl 6-bromohexanoate (585 mg, 1.3 mmol, 1.0 eq) in acetonitrile (10 mL), was added N-ethylbis(isopropyl)amine (1.13 mL, 6.48 mmol, 5 eq) and 11-methyldodecyl 6-(4-hydroxybutylamino)hexanoate (0.5 g. 1.3 mmol, 1.0 eq) to it. The reaction mixture was stirred at 95° C. for 48 h. The progress of reaction was monitored by TLC/ELSD (66% product formation in reaction mixture by ELSD). The reaction mixture was concentrated then quenched with saturated sodium bicarbonate solution (20 ml) and extracted with dichloromethane (20 mL×3). The resulting organic layer was dried over sodium sulphate, and concentrated under reduce pressure, and the crude was purified by flash column chromatography (SiO₂: 0-6% methanol in dichloromethane), to give 1-fluoroheptadecan-9-yl 6-((4-hydroxybutyl)(6-((11-methyldodecyl)oxy)-6-oxohexyl)amino) hexanoate (300 mg, Yield=30.6%) as a colorless liquid. The compound was dissolved in dichloromethane (10 ml) and filtered through Hydrophobic PTFE Syringe Filter, 25 mm*0.45 μm by glass syringe to get desired compound. ¹H-NMR (400 MHz, CDCl₃-d3): δ 4.88-4.82 (m, 1H), 4.48 (t, J=6.0 Hz, 1H), 4.37 (t, J=6.0 Hz, 1H), 4.04 (t, J=6.8 Hz, 2H), 3.54 (bs, 2H), 2.45-2.41 (bs, 6H), 2.31-2.26 (m, 4H), 1.72-1.61 (m, 14H), 1.54-1.45 (m, 10H), 1.39-1.25 (m, 40H), 1.16-1.13 (m, 2H), 0.89-0.85 (m, 9H). ¹⁹F-NMR (375 MHz, CDCl₃-d₃): −217.99 ELSD analysis: Purity 99.89%, Calculated C₄₆H₉₁FNO₅, [M+H⁺]=756.68, Observed=756.65 (m/z, M+H⁺).
Example 178. Synthesis of Compound 64: 11-methyldodecyl 6-((6-((1-fluoroheptadecan-9-yl)oxy)-6-oxohexyl)(4-hydroxybutyl)amino)-2-methylhexanoate
Step 1: Synthesis of 3-(6-bromohexanoyl)oxazolidin-2-one

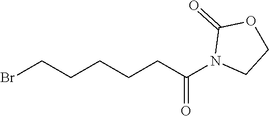 [SMILES: O=C(CCCCCBr)N1CCOC1=O]

To a stirred solution of 6-bromohexanoic acid (10 g, 51.3 mmol, 1.0 eq) in dichloromethane (400 ml) and dimethylformamide (2 ml), was added oxalyl dichloride (22 mL, 256 mmol, 5.0 eq) at 0° C. under nitrogen atmosphere and stirred at r.t. for 16 h. After completion of the reaction (monitored by TLC using MeOH to formation Methyl ester). The reaction mixture was concentrated to get 6-bromohexanoyl chloride (10.2 g, Yield=93.91%) as a yellow solid. This intermediate was used in the next step without further purification. To a stirred solution of 1,3-oxazolidin-2-one (4.08 g, 46.8 mmol, 1.0 eq) in dichloromethane (150 ml) was added N,N-dimethyl-4-pyridylamine (1.14 g, 9.37 mmol, 0.2 eq.) and triethylamine (32.9 mL, 234 mmol, 5 eq) and stirred it to 15 min at 0° C. Then 6-bromohexanoyl chloride (10.2 g, 46.8 mmol, 1.0 eq) in dichloromethane (10 ml) was added dropwise into the reaction mixture and stirred reaction mixture overnight. The progress of reaction was monitored by TLC. The reaction was concentrated in vacuum which was dissolved with EtOAc (100 mL) and washed with 3% HCl (100 mL). The resulting organic layer was dried over Sodium sulphate, and concentrated under reduce pressure, and the crude was purified by flash column chromatography (SiO₂: 0-3% Ethyl acetate in Hexane) to get 3-(6-bromohexanoyl)-1,3-oxazolidin-2-one (8.9 g, Yield=71.94%) as a thick oil. ¹H NMR (400 MHz, CDCl₃-d3): δ 4.41 (t, J=8.0 Hz, 2H), 4.02 (t, J=8.0 Hz, 2H), 3.54 (t, J=6.8 Hz, 2H), 2.98-2.92 (m, 2H), 1.91-1.79 (m, 2H), 1.73-1.66 (m, 2H), 1.55-1.50 (m, 2H).
Step 2: Synthesis of 3-(6-bromo-2-methylhexanoyl)oxazolidin-2-one

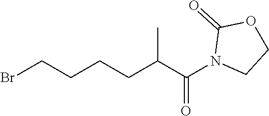

To a stirred solution of 3-(6-bromohexanoyl)-1,3-oxazolidin-2-one (15 g, 56.8 mmol, 1.0 eq) in tetrahydrofuran (135 ml) was added sodium bis(trimethylsilyl)azanide (13.5 g, 73.8 mmol, 1.3 eq) dropwise at −78° C. and the reaction mixture was stirred at same temperature for 1 hr, then iodomethane (17.7 mL, 284 mmol, 5 eq) (dissolved in THF) was added dropwise and the reaction mixture was stirred at −78° C. for 4 h. After completion of reaction (monitored by TLC), the reaction mixture was quenched with aq. NH₄Cl solution and extracted with ethyl acetate (100 mL×3), washed consecutively with 5% KHSO₄ (100 ml), saturated Na₂S₂O₃(100 ml) and brine (100 ml). The resulting organic layer was dried over Na₂SO₄, and concentrated under reduce pressure, and the crude was purified by flash column chromatography (SiO₂: 0-3% Ethyl acetate in Hexane), to get 3-(6-bromo-2-methylhexanoyl)-1,3-oxazolidin-2-one (9.5 g, Yield=60.1%) as a colorless liquid. ¹H-NMR (400 MHz, CDCl3-d3): δ 4.40 (t, J=8.0 Hz, 2H), 4.02 (t, J=8.0 Hz, 2H), 3.76-3.71 (m, 1H), 3.52 (t, J=6.8 Hz, 2H), 1.87-1.70 (m, 3H), 1.50-1.40 (m, 3H), 1.18 (d, J=7.2 Hz, 3H).
Step 3: Synthesis of 6-bromo-2-methylhexanoic acid

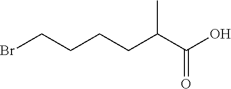

To a stirred solution of 3-(6-bromo-2-methylhexanoyl)-1,3-oxazolidin-2-one (5.2 g, 18.7 mmol, 1.0 eq.) in THF (90 ml) and water (30 mL), was added hydrogen peroxide (14.6 ml, 187 mmol, 10 eq.) and lithium hydroxide (1.34 g, 56.1 mmol, 3 eq.) to it. The reaction mixture was stirred for 3 h, after completion of reaction (monitored by TLC). The reaction mixture quenched with Na₂SO₃, acidified up to pH 2 with the help of 5% KHSO₄ and extracted with ethyl acetate (200 mL×3), The resulting organic layer was dried over Na₂SO₄, and concentrated under reduce pressure to get 8-bromo-2-methyloctanoic acid (2.8 g, Yield=71.63%) as a colorless liquid, which was used in the next step without further purification. ¹H NMR (400 MHz, CDCl₃-d3): δ 3.58-3.54 (m, 2H), 2.52-2.41 (m, 1H), 1.80-1.64 (m, 3H), 1.51-1.41 (m, 3H), 1.18 (d, J=7.2 Hz, 3H).
Step 4: Synthesis of 11-methyldodecyl 6-bromo-2-methylhexanoate

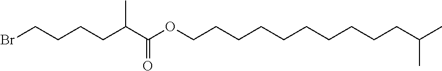 [SMILES: CC(C)CCCCCCCCCCOC(=O)C(C)CCCCBr]

To a stirred solution of 6-bromo-2-methylhexanoic acid (2.5 g, 12 mmol, 1 eq) in dichloromethane (55 ml), was added 4-(dimethylamino)pyridin-1-ium (2.19 g, 17.9 mmol, 1.5 eq) and {3-[cyano(ethyl)amino]propyl}dimethylazanium chloride (5.73 g, 29.9 mmol, 2.5 eq) and stirred for 15 min, then 11-methyl-1-dodecanol (2.4 g. 12 mmol, 1.0 eq) was added. The reaction mixture was stirred at r.t. for 16 h. The progress of reaction was monitored by TLC (SM was consumed). Water (500 mL) was added to the reaction mixture, and extracted with DCM (3×250 mL). The resulting organic layer was dried over sodium sulphate, and concentrated under reduce pressure, and the crude was purified by flash column chromatography (SiO₂: 0-20% Ethyl acetate in Hexanes), to get a 11-methyldodecyl 6-bromo-2-methylhexanoate (3.1 g, Yield=66.23%) as a colorless liquid. ¹H-NMR (400 MHz, CDCl3-d3): δ 4.06 (t, J=6.8 Hz, 2H), 3.52 (t, J=6.4 Hz, 2H), 2.45-2.37 (m, 1H), 1.80-1.72 (m, 2H), 1.70-1.60 (m, 3H), 1.52-1.39 (m, 4H), 1.37-1.20 (m, 14H), 1.19-1.12 (m, 5H), 0.86 (d, J=6.4 Hz, 6H).
Step 5: Synthesis of 1-fluoroheptadecan-9-yl 6-((4-hydroxybutyl)amino)hexanoate

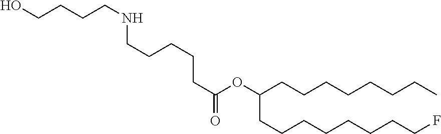 [SMILES: CCCCCCCCC(CCCCCCCCF)OC(=O)CCCCCNCCCCO]

To a stirred solution of 4-amino-1-butanol (0.7 g, 7.85 mmol, 1.0 eq) in acetonitrile (100 mL), was added 9-fluoro-1-octylnonyl 6-bromohexanoate (3.55 g, 7.85 mmol, 1.0 eq) and N-ethylbis(isopropyl)amine (3.42 μL, 2.5 eq., 19.6 mmol) to it. The reaction was stirred at 55° C. for 16 h. The progress of reaction was monitored by ELSD/TLC. The reaction mixture was concentrated under reduced pressure and the crude was purified by flash column chromatography (SiO₂: 0-10% Methanol in Dichloromethane) to get 9-fluoro-1-octylnonyl 6-(4-hydroxybutylamino)hexanoate (1.7 g, Yield=33.69%) as a brown liquid. ELSD analysis: Purity 98.28%, Calculated C₂₇H₅₅FNO₃, [M+H⁺]=460.41, Observed=460.30 (m/z, M+H⁺).
Step 6: Synthesis of 11-methyldodecyl 6-((6-((1-fluoroheptadecan-9-yl)oxy)-6-oxohexyl)(4-hydroxybutyl)amino)-2-methylhexanoate

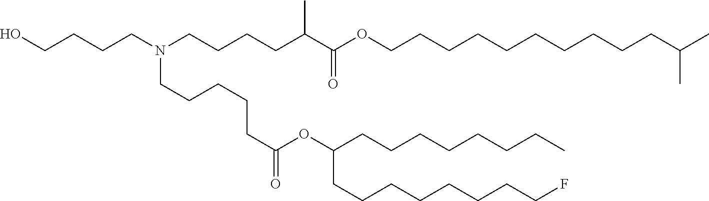 [SMILES: CCCCCCCCC(CCCCCCCCF)OC(=O)CCCCCN(CCCCO)CCCCC(C)C(=O)OCCCCCCCCCCC(C)C]

To a stirred solution of compound 9-fluoro-1-octylnonyl 6-(4-hydroxybutylamino)hexanoate (0.5 g, 1.09 mmol 1.0 eq) in acetonitrile (10 mL), was added N-ethylbis(isopropyl)amine (422 mg, 3.26 mmol, 3 eq) and 11-methyldodecyl 6-bromo-2-methylhexanoate (426 mg, 1.09 mmol, 1.0 eq) to it. The reaction mixture was stirred at 95° C. for 48 h. The progress of reaction was monitored by TLC/ELSD. The reaction mixture was concentrated then quenched with saturated sodium bicarbonate solution (20 ml) and extracted with dichloromethane (20 mL×3). The resulting organic layer was dried over sodium sulphate, and concentrated under reduce pressure. The crude was purified by flash column chromatography (SiO₂: 0-6% methanol in dichloromethane), to give 11-methyldodecyl 6-{[5-(9-fluoro-1-octylnonyloxycarbonyl) pentyl](4-hydroxybutyl)amino}-2-methylhexanoate (150 mg, Yield=17.91%) as a pale yellow liquid. The compound was dissolved in dichloromethane (10 ml) and filtered through Hydrophobic PTFE Syringe Filter, 25 mm*0.45 μm by glass syringe to get desired compound. 1H-NMR (400 MHz, CDCl3-d3): δ 4.88-4.82 (m, 1H), 4.48 (t, J=6.0 Hz, 1H), 4.37 (t, J=6.0 Hz, 1H), 4.05 (t, J=6.8 Hz, 2H), 3.55 (bs, 2H), 2.45-2.38 (bs, 6H), 2.30-2.26 (m, 2H), 1.72-1.61 (m, 12H), 1.54-1.45 (m, 8H), 1.39-1.25 (m, 42H), 1.16-1.13 (m, 5H), 0.89-0.85 (m, 9H). 19F-NMR (375 MHz, CDCl3-d3): −217.99 ELSD analysis: Purity 99.94%, Calculated C₄₇H₉₃FNO₅, [M+H+]=770.70, Observed=770.65 (m/z, M+H+).
Example 179. Synthesis of Compound Compound 65: 11-methyldodecyl 6-((6-((1-fluoroheptadecan-9-yl)oxy)-6-oxohexyl)(2-hydroxyethyl)amino)-2-methylhexanoate
Step 1: Synthesis of 1-fluoroheptadecan-9-yl 6-((2-hydroxyethyl)amino)hexanoate

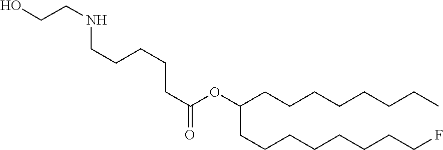 [SMILES: CCCCCCCCC(CCCCCCCCF)OC(=O)CCCCCNCCO]

To a stirred solution of 2-aminoethanol (0.5 g, 8.19 mmol, 1.0 eq) in acetonitrile (100 mL), was added 9-fluoro-1-octylnonyl 6-bromohexanoate (3.7 g. 8.19 mmol, 1.0 eq) and N-ethylbis(isopropyl)amine (3.56 μL, 20.5 mmol, 2.5 eq.) to it. The reaction was stirred at 55° C. for 16 h. The progress of reaction was monitored by ELSD/TLC. The reaction mixture was concentrated under reduced pressure and the crude was purified by flash column chromatography (SiO₂: 0-10% Methanol in Dichloromethane) to get 9-fluoro-1-octylnonyl 6-(2-hydroxyethylamino)hexanoate (1.1 g, Yield=31.13%) as a brown liquid. ELSD analysis: Purity 99.77%, Calculated C₂₅H₅₁FNO₃, [M+H⁺]=432.38, Observed=432.30 (m/z, M+H⁺).
Step 2: Synthesis of 11-methyldodecyl 6-((6-((1-fluoroheptadecan-9-yl)oxy)-6-oxohexyl)(2-hydroxyethyl)amino)-2-methylhexanoate

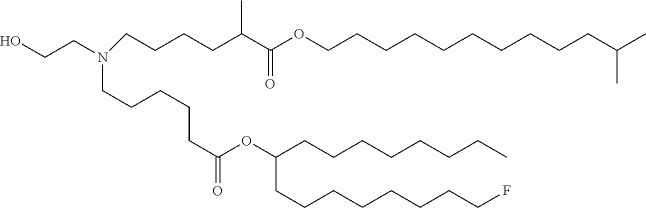 [SMILES: CCCCCCCCC(CCCCCCCCF)OC(=O)CCCCCN(CCO)CCCCC(C)C(=O)OCCCCCCCCCCC(C)C]

To a stirred solution of compound 9-fluoro-1-octylnonyl 6-(2-hydroxyethylamino)hexanoate (0.5 g, 1.16 mmol, 1.0 eq) in acetonitrile (10 mL), was added N-ethylbis(isopropyl)amine (449 mg, 3.47 mmol, 3 eq) and 11-methyldodecyl 6-bromo-2-methylhexanoate (453 mg, 1.16 mmol, 1.0 eq) to it. The reaction mixture was stirred at 95° C. for 48 h. The progress of reaction was monitored by TLC/ELSD. The reaction mixture was concentrated then quenched with saturated sodium bicarbonate solution (20 ml) and extracted with dichloromethane (20 mL×3). The resulting organic layer was dried over sodium sulphate, concentrated under reduce pressure. The crude was purified by flash column chromatography (SiO₂: 0-6% methanol in dichloromethane), to give 11-methyldodecyl 6-{[5-(9-fluoro-1-octylnonyloxycarbonyl)pentyl](2-hydroxyethyl)amino}-2-methylhexanoate (130 mg, Yield=15.12%) as a pale yellow liquid. The compound was dissolved in dichloromethane (10 ml) and filtered through Hydrophobic PTFE Syringe Filter, 25 mm*0.45 μm by glass syringe to get desired compound. ¹H-NMR (400 MHz, CDCl₃-d3): δ 4.89-4.82 (m, 1H), 4.48 (t, J=6.0 Hz, 1H), 4.37 (t, J=6.0 Hz, 1H), 4.05 (t, J=6.8 Hz, 2H), 3.52 (bs, 2H), 2.58-2.56 (m, 2H), 2.44-2.38 (bs, 5H), 2.30-2.26 (t, J=7.6 Hz, 2H), 1.71-1.58 (m, 8H), 1.54-1.25 (m, 49H), 1.14-1.13 (m, 5H), 0.89-0.85 (m, 9H). ¹⁹F-NMR (375 MHz, CDCl₃-d₃): −218.00 ELSD analysis: Purity 99.82%, Calculated C₄₅H₈₉FNO₅, [M+H⁺]=742.70, Observed=742.65 (m/z, M+H⁺).
Example 180. Synthesis of Compound 66:9-methyldecyl 8-((8-((1-fluoroheptadecan-9-yl)oxy)-8-oxooctyl)(2-hydroxyethyl)amino)-2-methyloctanoate

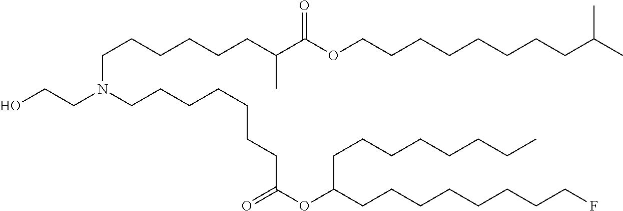 [SMILES: CCCCCCCCC(CCCCCCCCF)OC(=O)CCCCCCCN(CCO)CCCCCCC(C)C(=O)OCCCCCCCCC(C)C]

To a stirred solution of 9-fluoro-1-octylnonyl 8-(2-hydroxyethylamino)octanoate (550 mg, 1.2 mmol. 1.0 eq) in acetonitrile (10 mL) was added 9-methyldecyl 8-bromo-2-methyloctanoate (468 mg, 1.2 mmol, 1.0 eq) and N-ethylbis(isopropyl)amine (0.627 mL, 3.59 mmol, 3 eq). The reaction was stirred at 100° C. for 48 h. The progress of reaction was monitored by ELSD/TLC (72% product form in reaction mixture by ELSD). The reaction mixture was concentrated under reduced pressure and the crude was purified by flash column chromatography (SiO₂: 0-10% Methanol in Dichloromethane) to get 9-methyldecyl 8-{[7-(9-fluoro-1-octylnonyloxycarbonyl)heptyl](2-hydroxyethyl)amino}-2-methyloctanoate (120 mg) as pale yellow liquid. The compound (120 mg) was obtained after column chromatography was dissolved in dichloromethane (10 mL) and added anhydrous potassium carbonate (5 eq) under nitrogen atmosphere and stirred vigorously for 2 h. Then, reaction mixture was filtered through hydrophobic PTFE syringe filter (25 mm×0.45 μm) using glass syringe to get 9-methyldecyl 8-{[7-(9-fluoro-1-octylnonyloxycarbonyl)heptyl](2-hydroxyethyl)amino}-2-methyloctanoate (111 mg, Yield=12.05%) as a pale yellow liquid. ¹H-NMR (400 MHz, CDCl₃-d3): δ 4.88-4.82 (m, 1H), 4.48 (t, J=6.0 Hz, 1H), 4.37 (t, J=6.0 Hz, 1H), 4.05 (t, J=6.8 Hz, 2H), 3.54 (b, 2H), 2.59 (b, 2H), 2.45-2.36 (m, 4H), 2.27 (t, J=7.6 Hz, 2H), 1.72-1.59 (m, 8H), 1.54-1.25 (m, 55H), 1.15-1.12 (m, 5H), 0.89-0.85 (m, 9H). ¹⁹F-NMR (375 MHz, CDCl₃-d₃): −217.99. ELSD analysis: Purity 99.82%, Calculated C₄₇H₉₃FNO₅, [M+H]=770.70, Observed=770.65 (m/z, M+H⁺).
Example 181. Synthesis of Compound 67: 9-methyldecyl 8-((8-((1-fluoroheptadecan-9-yl)oxy)-8-oxooctyl)(4-hydroxybutyl)amino)-2-methyloctanoate
Step 1: Synthesis of 1-fluoroheptadecan-9-yl 8-((4-hydroxybutyl)amino)octanoate

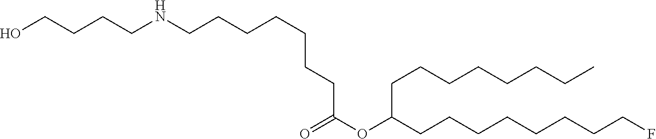 [SMILES: CCCCCCCCC(CCCCCCCCF)OC(=O)CCCCCCCNCCCCO]

To a stirred solution of 9-fluoro-1-octylnonyl 8-bromooctanoate (1.3 g, 2.71 mmol, 1.0 eq) in acetonitrile (15 mL) was added 4-amino-1-butanol (242 mg, 2.71 mmol, 1.0 eq) and N-ethylbis(isopropyl)amine (568 μL, 3.25 mmol, 1.2 eq). The reaction was stirred at 70° C. for 6 days. The progress of reaction was monitored by ELSD/TLC (SM was consumed). The reaction mixture was concentrated under reduced pressure and the crude was purified by flash column chromatography (SiO₂: 0-10% Methanol in Dichloromethane) to give 9-fluoro-1-octylnonyl 8-(4-hydroxybutylamino)octanoate (560 mg, Yield=42.35%) as a white gummy solid. ELSD analysis: Purity 99.72%, Calculated C₂₉H₅₉FNO₃, [M+H]=488.44, Observed=488.35 (m/z, M+H+).
Step 2: Synthesis of 9-methyldecyl 8-((8-((1-fluoroheptadecan-9-yl)oxy)-8-oxooctyl)(4-hydroxybutyl)amino)-2-methyloctanoate

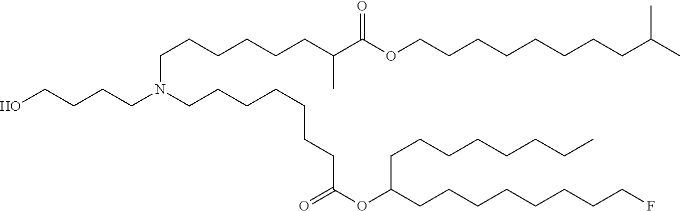 [SMILES: CCCCCCCCC(CCCCCCCCF)OC(=O)CCCCCCCN(CCCCO)CCCCCCC(C)C(=O)OCCCCCCCCC(C)C]

To a stirred solution of 9-fluoro-1-octylnonyl 8-(4-hydroxybutylamino)octanoate (550 mg, 1.12 mmol, 1.0 eq) in acetonitrile (10 mL) was added 9-methyldecyl 8-bromo-2-methyloctanoate (441 mg, 1.12 mmol, 1.0 eq) and N-ethylbis(isopropyl)amine (0.590 mL, 3.38 mmol, 3 eq). The reaction was stirred at 100° C. for 72 h. The progress of reaction was monitored by ELSD/TLC (54% product form in reaction mixture by ELSD). The reaction mixture was concentrated under reduced pressure and the crude was purified by flash column chromatography (SiO₂: 0-10% Methanol in Dichloromethane) to get 9-methyldecyl 8-{[7-(9-fluoro-1-octylnonyloxy carbonyl)heptyl](4-hydroxybutyl)amino}-2-methyloctanoate (135 mg) as pale yellow liquid. The compound (135 mg) was obtained after column chromatography was dissolved in dichloromethane (10 mL) and added anhydrous potassium carbonate (5 eq) under nitrogen atmosphere and stirred vigorously for 2 h. Then, reaction mixture was filtered through hydrophobic PTFE syringe filter (25 mm×0.45 μm) using glass syringe to get 9-methyldecyl 8-{[7-(9-fluoro-1-octylnonyloxycarbonyl)heptyl](4-hydroxybutyl)amino}-2-methyloctanoate (125 mg, Yield=13.9%) as a pale yellow liquid. ¹H-NMR (400 MHz, CDCl₃-d3): δ 4.88-4.82 (m, 1H), 4.48 (t, J=6.0 Hz, 1H), 4.37 (t, J=6.0 Hz, 1H), 4.05 (t, J=6.8 Hz, 2H), 3.56 (b, 2H), 2.61-2.37 (m, 6H), 2.27 (t, J=7.6 Hz, 2H), 1.72-1.59 (m, 12H), 1.54-1.47 (m, 8H), 1.42-1.28 (m, 47H), 1.15-1.12 (m, 5H), 0.89-0.85 (m, 9H). ¹⁹F-NMR (375 MHz, CDCl₃-d₃): −217.99. ELSD analysis: Purity 99.36%, Calculated C₄₉H₉₇FNO₅, [M+H]=798.73, Observed=798.75 (m/z, M+H⁺).
Example 182. Synthesis of Compound 68: 9-methyldecyl 8-((8-(heptadecan-9-yloxy)-8-oxooctyl)(4-hydroxybutyl)amino)-2-methyloctanoate

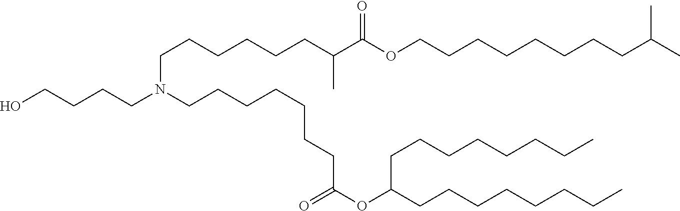 [SMILES: CCCCCCCCC(CCCCCCCC)OC(=O)CCCCCCCN(CCCCO)CCCCCCC(C)C(=O)OCCCCCCCCC(C)C]

To a stirred solution of compound 9-methyldecyl 8-bromo-2-methyloctanoate (431 mg, 1.1 mmol, 1.0 eq) in acetonitrile (10 mL), was added N-ethylbis(isopropyl)amine (577 μL, 3.3 mmol, 3 eq) and 1-octylnonyl 8-(4-hydroxybutylamino)octanoate (517 mg, 1.1 mmol, 1.0 eq) to it. The reaction mixture was stirred at 95° C. for 48 h. The progress of reaction was monitored by TLC/ELSD (70% product formation in reaction mixture by ELSD). The reaction mixture was concentrated then quenched with saturated sodium bicarbonate solution (20 ml) and extracted with dichloromethane (20 mL×3). The resulting organic layer was dried over sodium sulphate, and concentrated under reduce pressure, and the crude was purified by flash column chromatography (SiO₂: 0-6% methanol in dichloromethane), to give 9-methyldecyl 8-((8-(heptadecan-9-yloxy)-8-oxooctyl)(4-hydroxybutyl)amino)-2-methyloctanoate (250 mg, Yield=29.11%) as a colorless liquid. The compound was dissolved in dichloromethane (10 ml) and filtered through Hydrophobic PTFE Syringe Filter, 25 mm*0.45 μm by glass syringe to get desired compound. ¹H-NMR (400 MHz, CDCl₃-d₃): δ 4.87-4.84 (m, 1H), 4.05 (t, J=6.8 Hz, 2H), 3.54 (bs, 2H), 2.43-2.37 (bs, 6H), 2.27 (t, J=7.6 Hz, 2H), 1.65-1.59 (m, 10H), 1.52-1.47 (m, 10H), 1.30-1.25 (m, 48H), 1.16-1.21 (m, 5H), 0.89-0.85 (m, 12H). ELSD analysis: Purity 99.43%, Calculated C₄₇H₉₄NO₅, [M+H⁺]=780.74, Observed=780.70 (m/z, M+H⁺).
Example 183. Synthesis of Compound 69: 7-((7-((10-fluoro-2-octyldecanoyl)oxy)heptyl)(4-hydroxybutyl)amino)heptyl 10-methylundecanoate
Step 1: Synthesis of 7-((4-hydroxybutyl)amino)heptyl 10-methylundecanoate

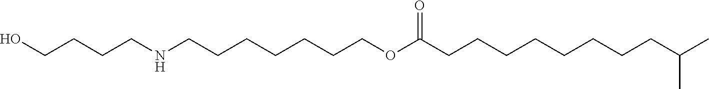 [SMILES: CC(C)CCCCCCCCC(=O)OCCCCCCCNCCCCO]

To a stirred solution of 4-amino-1-butanol (472 mg, 5.3 mmol, 1.0 eq) in acetonitrile (25 mL), was added 7-bromoheptyl 10-methylundecanoate (2 g, 5.3 mmol, 1.0 eq) and N-ethylbis(isopropyl)amine (1.85 mL, 10.6 mmol, 2.0 eq) to it. The reaction was stirred at 55° C. for 48 h. The progress of reaction was monitored by ELSD/TLC (42% product form in reaction mixture by ELSD). The reaction mixture was concentrated under reduced pressure and the crude was purified by flash column chromatography (SiO₂: 0-10% Methanol in Dichloromethane) to give 7-(4-hydroxybutylamino)heptyl 10-methylundecanoate (0.6 g, Yield=29.36%) as a White solid. ELSD analysis: Purity 99.44%, Calculated C₂₃H₄₈NO₃, [M+H+]=386.36, Observed=386.30 (m/z, M+H+).

Step 2: Synthesis of 7-((7-((10-fluoro-2-octyldecanoyl)oxy)heptyl)(4-hydroxybutyl)amino)heptyl 10-methylundecanoate

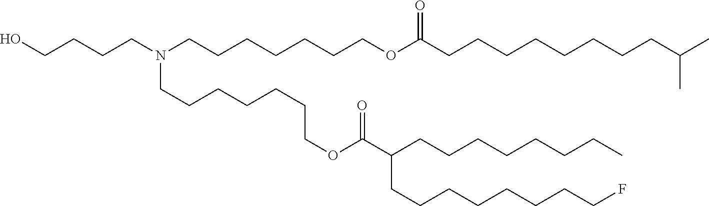 [SMILES: CCCCCCCCC(CCCCCCCCF)C(=O)OCCCCCCCN(CCCCO)CCCCCCCOC(=O)CCCCCCCCC(C)C]

To a stirred solution of compound 7-(4-hydroxybutylamino)heptyl 10-methylundecanoate (0.5 g, 1.3 mmol, 1.0 eq) in acetonitrile (10 mL), was added N-ethylbis(isopropyl)amine (0.692 μL, 3.89 mmol, 3 eq) and 7-bromoheptyl 10-fluoro-2-octyldecanoate (622 mg, 1.3 mmol, 1.0 eq) to it. The reaction mixture was stirred at 95° C. for 48 h. The progress of reaction was monitored by TLC/ELSD (55% product formation in reaction mixture by ELSD). The reaction mixture was concentrated to get crude. The crude was purified through combi-flash chromatography and compound eluted in 0-8% MeOH in DCM. The desired fraction was collected and evaporated to get of 300 mg compound of 90% purity. The compound was submitted for reverse phase HPLC. The desired fraction were collected, evaporated and lyophilized to get 7-{[7-(9-fluoro-1-octylnonylcarbonyloxy)heptyl](4-hydroxybutyl)amino}heptyl 10-methyl-undecanoate (101 mg, 9.93%) as a colorless liquid. The compound was dissolved in dichloromethane (10 ml) and filtered through Hydrophobic PTFE Syringe Filter, 25 mm*0.45 μm by glass syringe to get desired compound. ¹H-NMR (400 MHz, CDCl₃-d₃): δ 4.48 (t, J=6.0 Hz, 1H), 4.36 (t, J=6.0 Hz, 1H), 4.07-4.03 (m, 4H), 3.58 (m, 2H), 2.71-2.39 (m, 4H), 2.33-2.26 (m, 3H), 1.73-1.60 (m, 9H), 1.51-1.42 (m, 10H), 1.34-1.24 (m, 48H), 1.14-1.13 (m, 2H), 0.88-0.84 (m, 9H). ¹⁹F-NMR (375 MHz, CDCl₃-d₃): −217.97. ELSD analysis: Purity 99.68%, Calculated C₄₈H₉₅FNO₅, [M+H⁺]=784.71, Observed=784.70 (m/z, M+H⁺).
Example 184. Synthesis of Compound 70:1-fluoroheptadecan-9-yl 6-((2-hydroxyethyl)(6-((11-methyldodecyl)oxy)-6-oxohexyl)amino)hexanoate
Step 1: Synthesis of 11-methyldodecyl 6-((2-hydroxyethyl)amino)hexanoate

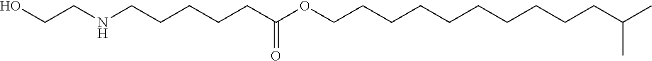 [SMILES: CC(C)CCCCCCCCCCOC(=O)CCCCCNCCO]

To a stirred solution of 11-methyldodecyl 6-bromohexanoate (1.2 g, 3.18 mmol, 1.0 eq) in acetonitrile (30 mL), was added N-ethylbis(isopropyl)amine (1.67 mL, 9.54 mmol, 3.0 eq) and 2-aminoethanol (194 mg, 3.18 mmol, 1.0 eq) to it. The reaction mixture was stirred at 55° C. for 16 h. The progress of reaction was monitored by TLC/ELSD. The reaction mixture was concentrated to get the crude mixture. The crude mixture was purified by combi-flash chromatography (SiO₂, 6-7% Methanol in DCM). The desired fractions were collected and evaporated to get 11-methyldodecyl 6-(2-hydroxyethylamino)hexanoate (0.5 g, Yield=43.98%) as a colorless liquid. ELSD analysis: Purity 98.89%, Calculated C₂₁H₄₄NO₃, [M+H⁺]=358.32, Observed=358.40 (m/z, M+H⁺).
Step 2: Synthesis of 1-fluoroheptadecan-9-yl 6-((2-hydroxyethyl)(6-((11-methyldodecyl)oxy)-6-oxohexyl)amino)hexanoate

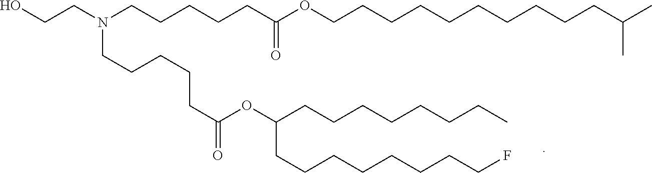 [SMILES: CCCCCCCCC(CCCCCCCCF)OC(=O)CCCCCN(CCO)CCCCCC(=O)OCCCCCCCCCCC(C)C]

To a stirred solution of 11-methyldodecyl 6-(2-hydroxyethylamino)hexanoate (0.5 g, 1.4 mmol, 1.0 eq) in acetonitrile (10 mL), was added 9-fluoro-1-octylnonyl 6-bromohexanoate (631 mg, 1.4 mmol, 1.0 eq) and N-ethylbis(isopropyl)amine (631 μL, 6.99 mmol, 5.0 eq). The reaction was stirred at 85° C. for 48 h. The progress of reaction was monitored by ELSD/TLC (87% product form in reaction mixture by ELSD). After completion the reaction, reaction mixture was concentrated under reduced pressure and diluted with water (50 mL) and extracted with DCM (2×40 mL). The organic layer collected and again washed with aq. sodium bicarbonate solution (2×50 mL). The organic layer dried over sodium sulphate, filtered and concentrated to get crude. The crude was purified by flash column chromatography (SiO₂: 0-10% Methanol in Dichloromethane) to get 1-fluoroheptadecan-9-yl 6-((2-hydroxyethyl)(6-((11-methyldodecyl)oxy)-6-oxohexyl)amino)hexanoate (250 mg) as colourless liquid. The compound (250 mg) was obtained after column chromatography was dissolved in dichloromethane (10 mL) and was filtered through hydrophobic PTFE syringe filter (25 mm×0.45 μm) using glass syringe to get 1-fluoroheptadecan-9-yl 6-((2-hydroxyethyl)(6-((11-methyldodecyl)oxy)-6-oxohexyl)amino)hexanoate (215 mg, Yield=21.05%) as a colourless liquid. ¹H-NMR (400 MHz, CDCl₃-d₃): δ 4.87-4.84 (m, 1H), 4.48 (t, J=6.0 Hz, 1H), 4.37 (t, J=6.0 Hz, 1H), 4.05 (t, J=6.8 Hz, 2H), 3.65 (b, 2H), 2.77-2.42 (m, 5H), 2.32-2.25 (m, 4H), 1.72-1.46 (m, 18H), 1.39-1.19 (m, 43H), 0.89-0.85 (m, 9H). ¹⁹F-NMR (375 MHz, CDCl₃-d₃): −217.99, ELSD analysis: Purity 99.94%, Calculated C₄₄H₈₇FNO₅, [M+H⁺]=728.65, Observed=728.65 (m/z, M+H).
Example 185. Synthesis of Compound 71: 10-fluoro-2-hexyldecyl 8-((4-hydroxybutyl)(8-(nonyloxy)-8-oxooctyl)amino)octanoate
Step 1: Synthesis of 10-hydroxydecyl acetate

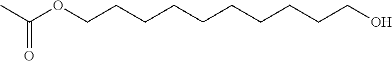

To a stirred solution of 1,10-decanediol (100 g, 574 mmol, 1.0 eq) in tetrahydrofuran (1000 mL), pyridine (92.6 mL, 1.15 mol, 2 eq) was added acetyl acetate (56.3 ml, 574 mmol, 1.0 eq) at 0° C. The reaction was stirred at 25° C. for 16 h. The progress of reaction was monitored by ELSD/TLC. The reaction mixture was quenched with aqueous sodium bicarbonate solution (250 ml) and extracted with ethyl acetate (5×500 ml). The organic layer was dried over sodium sulphate, filtered and concentrated to get crude. The crude was purified by flash column chromatography (silica gel, 0-20% gradient of ethyl acetate in hexane) to yield 10-hydroxydecyl acetate (51 g, Yield=41.09%) as a colorless liquid. ¹H NMR (400 MHz, CDCl₃-d3): δ 4.04 (t, J=6.8 Hz, 2H), 3.63 (t, J=6.8 Hz, 2H), 2.03 (s, 3H), 1.64-1.52 (m, 4H), 1.28 (b, 12H).
Step 2: Synthesis of 10-fluorodecyl acetate

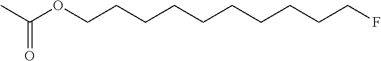

To a stirred solution of 10-hydroxydecyl acetate (51 g, 236 mmol, 1.0 eq) in dichloromethane (600 ml) was cooled to −78° C. and added N,N-diethyl(trifluorothio)amine (95 mL, 472 mmol, 2.0 eq) dropwise. The reaction mixture was stirred at room temperature and stirring for 16 h. The progress of reaction was monitored by ELSD/TLC. The reaction mass was cooled to −78° C., added dropwise aqueous sodium bicarbonate solution (35 mL) and extracted with ethyl acetate (3×100 mL). The organic layer was collected dried over sodium sulphate, filtered and evaporated to dryness. The crude was purified by flash column chromatography (silica gel, 0-40% gradient of ethyl acetate in hexane) to obtain 10-fluorodecyl acetate (35 g, Yield=45.4%) as white solid. ¹H NMR (400 MHz, CDCl₃-d3): δ 4.51 (t, J=6.0 Hz, 1H), 4.39 (t, J=6.4 Hz, 1H), 4.06 (t, J=6.8 Hz, 2H), 2.06 (s, 3H), 1.76-1.58 (m, 4H), 1.42-1.28 (m, 12H).
Step 3: Synthesis of 10-fluorodecan-1-ol

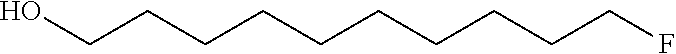 [SMILES: OCCCCCCCCCCF]

To a stirred solution of 10-fluorodecyl acetate (35 g, 160 mmol, 1.0 eq) in tetrahydrofuran (200 ml) was cooled to −78° C. and added Lithium aluminium hydride (12.2 g, 321 mmol, 2 eq) dropwise. After complete addition, reaction mixture was stirred at 25° C. for 16 h. The progress of reaction was monitored by ELSD/TLC. The reaction mass was cooled to −10° C. and quenched with aqueous ammonium chloride (100 ml). Then, reaction mass was filtered through celite bed. The filtrate was collected and evaporated under reduced pressure. The crude was purified by flash column chromatography (silica gel, 0-12% gradient of ethyl acetate in hexane) to obtain 10-fluoro-1-decanol (24 g, Yield=84.91%) as a colorless liquid. ¹H NMR (400 MHz, CDCl₃-d3): δ 4.49 (t, J=6.0 Hz, 1H), 4.37 (t, J=6.0 Hz, 1H), 3.64 (q, J=5.6 Hz, 2H), 1.74-1.62 (m, 2H), 1.59-1.53 (m, 2H), 1.40-1.20 (m, 12H).
Step 4: Synthesis of 10-fluorodecanoic acid

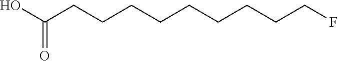 [SMILES: O=C(O)CCCCCCCCCF]

To a stirred solution of chromamethanetrione-sulfuric acid (108 mL, 709 mmol, 5 eq) and water (50 ml) was cooled to −10° C. and added a solution of 10-fluoro-1-decanol (25 g, 142 mmol, 1 eq) in acetone (250 ml) added dropwise to the solution. The reaction mixture was stirred for overnight at room temperature. The progress of reaction was monitored by TLC. Reaction mixture was dilute with water (1 l) and extracted with diethylether (5×500 ml). The resulting organic layer was dried over Na₂SO₄, and concentrated under reduce pressure to get the crude. The crude was purified by flash column chromatography (SiO₂: 0-40% Ethyl acetate in Hexanes) to get 10-fluorodecanoic acid (8 g, Yield=29.65%) as a white crystal. ¹H NMR (400 MHz, DMSO-d6): δ 11.97 (s, 1H), 4.83 (t, J=6.0 Hz, 1H), 4.36 (t, J=6.0 Hz, 1H), 2.19 (t, J=7.2 Hz, 2H), 1.67-1.56 (m, 2H), 1.50-1.47 (m 2H), 1.32-1.26 (m, 10H).
Step 5: Synthesis of 10-fluoro-2-hexyldecanoic acid

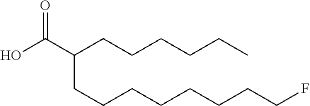 [SMILES: CCCCCCC(CCCCCCCCF)C(=O)O]

To a stirred solution of 10-fluorodecanoic acid (2 g, 10.5 mmol 1.0 eq) in tetrahydrofuran (60 mL) was added sodium hydride (505 mg, 12.6 mmol, 1.2 eq) at 0° C. and stirred for 30 minutes. Then, lithium bis(isopropyl)azanide (1.35 g, 12.6 mmol, 1.2 eq) was added to the reaction mixture at the −50° C. The reaction mixture was stirred at room temperature for 30 minutes and added 1-iodohexane (2.23 g, 10.5 mmol, 1.0 eq.). The reaction mixture was stirred at 45° C. for 16 h. The progress of reaction was monitored by TLC. The reaction mixture was poured into 1M HCl (250 mL) and extracted with ethyl acetate (3×250 mL). The resulting organic layer was dried over sodium sulfate, and concentrated under reduce pressure. The crude was purified by flash column chromatography (silica gel, 0-2% gradient of ethyl acetate in hexane) to obtain 10-fluoro-2-hexyldecanoic acid (1.2 g, Yield=41.6%) as a yellow liquid. ¹H NMR (400 MHz, CDCl₃-d3): δ 11.98 (b, 1H), 4.47 (t, J=6.4 Hz, 1H), 4.35 (t, J=6.0 Hz, 1H), 2.32-2.13 (m, 1H), 1.67-1.54 (m, 2H), 1.47-1.44 (m, 2H), 1.43-1.20 (m, 20H), 0.93 (t, J=6.4 Hz, 3H).
Step 6: Synthesis of 10-fluoro-2-hexyldecan-1-ol

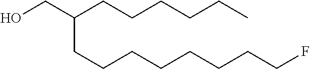 [SMILES: CCCCCCC(CO)CCCCCCCCF]

To a stirred solution of 10-fluoro-2-hexyldecanoic acid (1.2 g, 4.37 mmol, 1.0 eq) in tetrahydrofuran (25 mL) was added Lithium aluminium hydride 1.0 M in THF (332 mg, 8.75 mmol, 2.0 eq) dropwise at −78° C. The reaction mixture was stirred at room temperature for 3 hr. After completion of reaction (monitored by TLC). The reaction mixture was quenched with aq. ammonium chloride solution and extracted with ethyl acetate (3×50 mL). The organic layer was collected and dried over sodium sulphate, filtered and concentrated under vacuum to get the crude mixture. The crude product was purified through combi-flash chromatography (SiO₂: 0-10% Ethyl acetate in Hexane), to get 10-fluoro-2-hexyl-1-decanol (0.8 g, Yield=70.50%) as a colorless liquid. ¹H NMR (400 MHz, DMSO-d6): δ 4.47 (t, J=6.0 Hz, 1H), 4.35 (t, J=6.0 Hz, 1H), 4.29 (t, J=5.2 Hz, 1H), 3.26 (t, J=4.8 Hz, 2H), 1.66-1.56 (m, 2H), 1.29-1.13 (s, 23H), 0.85 (t, J=6.4 Hz, 3H).
Step 7: Synthesis of 10-fluoro-2-hexyldecyl 8-bromooctanoate

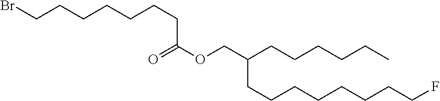 [SMILES: CCCCCCC(CCCCCCCCF)COC(=O)CCCCCCCBr]

To a stirred solution of 8-bromooctanoic acid (1.03 g, 4.61 mmol, 1.5 eq) in dichloromethane (40 ml), was added 4-(dimethylamino)pyridin-1-ium (563 mg, 4.61 mmol, 1.5 eq) and {3-[cyano(ethyl)amino]propyl}dimethylazanium chloride (1.77 g, 9.22 mmol, 3 eq). The reaction mixture was stirred for 15 min and added 10-fluoro-2-hexyl-1-decanol (0.8 g, 3.07 mmol, 1.0 eq). The reaction mixture was stirred at r.t. for 16 h. The progress of reaction was monitored by TLC (SM was consumed). Water (100 mL) was added to the reaction mixture and extracted with DCM (3×100 mL). The resulting organic layer was dried over sodium sulphate, and concentrated under reduce pressure. The crude was purified by flash column chromatography (SiO₂: 0-20% Ethyl acetate in Hexanes), to get a 10-fluoro-2-hexyldecyl 8-bromooctanoate (1 g, Yield=69.93%) as a colorless liquid. ¹H-NMR (400 MHz, CDCl₃-d3): δ 4.49 (t, J=6.0 Hz, 1H), 4.37 (t, J=6.0 Hz, 1H), 3.97 (d, J=5.2 Hz, 2H), 3.39 (t, J=6.8 Hz, 2H), 2.29 (t, J=7.2 Hz, 2H), 1.88-1.81 (m, 2H), 1.74-1.62 (m, 5H), 1.43-1.27 (m, 28H), 0.89-0.86 (m, 3H).
Step 8: Synthesis of nonyl 8-((4-hydroxybutyl)amino)octanoate

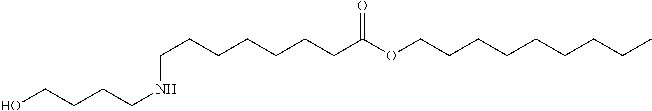 [SMILES: CCCCCCCCCOC(=O)CCCCCCCNCCCCO]

To a stirred solution of nonyl 8-bromooctanoate (4 g, 11.4 mmol, 1.0 eq) in acetonitrile (10.2 mL, 195 mmol) was added N-ethylbis(isopropyl)amine (4.44 g, 22.9 mmol, 3.0 eq) and 4-amino-1-butanol (1.02 g, 11.4 mmol, 1.0 eq). The reaction was stirred at 55° C. for 16 h. The progress of reaction was monitored by TLC/ELSD. The reaction mixture was concentrated to get the crude mixture. The crude mixture was purified by combi-flash chromatography (SiO₂, 6-7% Methanol in DCM). The desired fractions were collected and evaporated to get nonyl 8-(4-hydroxybutylamino)octanoate (1 g, Yield=33.78%) as a white solid. ELSD analysis: Purity 99.78%, Calculated C₂₁H₄₄NO₃, [M+H⁺]=358.33, Observed=358.30 (m/z, M+H+).
Step 9: Synthesis of 10-fluoro-2-hexyldecyl 8-((4-hydroxybutyl)(8-(nonyloxy)-8-oxooctyl)amino)octanoate

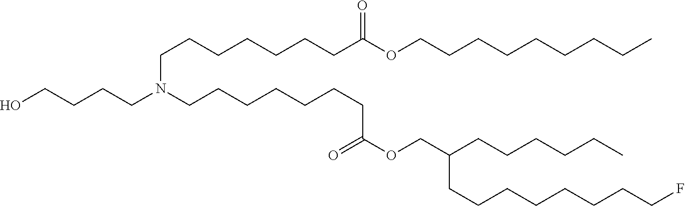 [SMILES: CCCCCCCCCOC(=O)CCCCCCCN(CCCCO)CCCCCCCC(=O)OCC(CCCCCC)CCCCCCCCF]

To a stirred solution of 10-fluoro-2-hexyldecyl 8-bromooctanoate (651 mg, 1.4 mmol, 1.0 eq) in acetonitrile (10 mL) was added nonyl 8-(4-hydroxybutylamino)octanoate (0.5 g, 1.4 mmol, 1.0 eq) and N-ethylbis(isopropyl)amine (0.747 μL, 4.19 mmol, 3 eq). The reaction was stirred at 95° C. for 24 h. The progress of reaction was monitored by ELSD/TLC (63% product form in reaction mixture by ELSD). The reaction mixture was concentrated under reduced pressure and the crude was purified by flash column chromatography (SiO₂: 0-10% Methanol in Dichloromethane) to get 7-((7-((10-fluoro-2-octyldecanoyl)oxy)heptyl)(4-hydroxybutyl)amino)heptyl 10-methylundecanoate (355 mg) as pale brown liquid. The compound (355 mg) was obtained after column chromatography was dissolved in dichloromethane (10 mL) and was filtered through hydrophobic PTFE syringe filter (25 mm×0.45 μm) using glass syringe to get 7-((7-((10-fluoro-2-octyldecanoyl)oxy)heptyl)(4-hydroxybutyl)amino)heptyl 10-methylundecanoate (348 mg, Yield=33.53%) as a pale brown liquid. ¹H-NMR (400 MHz, CDCl₃-d₃): δ 4.49 (t, J=6.0 Hz, 1H), 4.37 (t, J=6.0 Hz, 1H), 4.05 (t, J=6.8 Hz, 2H), 3.96 (d, J=5.6 Hz, 2H), 3.64 (b, 2H), 2.72 (b, 4H), 2.31-2.26 (m, 4H), 1.79-1.59 (m, 18H), 1.42-1.21 (m, 48H), 0.89-0.85 (m, 6H). ¹⁹F-NMR (375 MHz, CDCl₃-d₃): −217.99, ELSD analysis: Purity 99.75%, Calculated C₄₅H₈₉FNO₅, [M+H⁺]=742.66, Observed=742.70 (m/z, M+H+).
Example 186. Synthesis of Compound 72:10-fluoro-2-hexyldecyl 8-((2-hydroxyethyl)(8-(nonyloxy)-8-oxooctyl)amino)octanoate

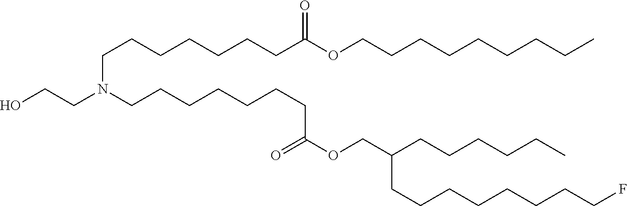 [SMILES: CCCCCCCCCOC(=O)CCCCCCCN(CCO)CCCCCCCC(=O)OCC(CCCCCC)CCCCCCCCF]

To a stirred solution of 10-fluoro-2-hexyldecyl 8-bromooctanoate (550 mg, 1.18 mmol, 1.0 eq) in acetonitrile (10 mL), was added nonyl 8-(2-hydroxyethylamino)octanoate (389 mg, 1.18 mmol, 1.0 eq) and N-ethylbis(isopropyl)amine (1.03 mL, 5.91 mmol, 5.0 eq). The reaction was stirred at 100° C. for 48 h. The progress of reaction was monitored by ELSD/TLC (82% product form in reaction mixture by ELSD). After completion the reaction, reaction mixture was concentrated under reduced pressure and diluted with water (50 mL) and extracted with DCM (2×40 mL). The organic layer collected and again washed with aq. sodium bicarbonate solution (2×50 mL). The organic layer dried over sodium sulphate, filtered and concentrated to get crude. The crude was purified by flash column chromatography (SiO₂: 0-10% Methanol in Dichloromethane) to get 10-fluoro-2-hexyldecyl 8-((2-hydroxyethyl)(8-(nonyloxy)-8-oxooctyl)amino)octanoate (215 mg) as colourless liquid. The compound (210 mg) was obtained after column chromatography was dissolved in dichloromethane (10 mL) and was filtered through hydrophobic PTFE syringe filter (25 mm×0.45 μm) using glass syringe to get 10-fluoro-2-hexyldecyl 8-((2-hydroxyethyl)(8-(nonyloxy)-8-oxooctyl)amino)octanoate (210 mg, Yield=24.89%) as a colourless liquid. ¹H-NMR (400 MHz, CDCl₃-d₃): δ 4.49 (t, J=6.0 Hz, 1H), 4.37 (t, J=6.0 Hz, 1H), 4.05 (t, J=6.8 Hz, 2H), 3.96 (t, J=6.0 Hz, 2H), 3.64 (b, 2H), 2.71 (b, 2H), 2.59 (b, 3H), 2.31-2.26 (m, 4H), 1.74-1.67 (m, 2H), 1.64-1.57 (m, 8H), 1.53 (b, 4H), 1.43-1.18 (m, 46H), 0.89-0.86 (m, 6H). ¹⁹F-NMR (375 MHz, CDCl₃-d₃): −217.99, ELSD analysis: Purity 99.83%, Calculated C₄₃H₈₅FNO₅, [M+H⁺]=714.63, Observed=714.65 (m/z, M+H⁺).
Example 187. Synthesis of Compound 73: 9-methyldecyl 8-((8-((10-fluoro-2-hexyldecyl)oxy)-8-oxooctyl)(2-hydroxyethyl)amino)-2-methyloctanoate
Step 1: Synthesis of 9-methyldecyl 8-((2-hydroxyethyl)amino)-2-methyloctanoate

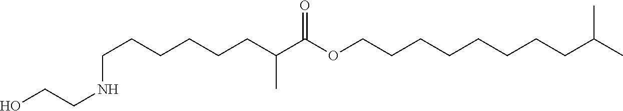 [SMILES: CC(C)CCCCCCCCOC(=O)C(C)CCCCCCNCCO]

To a stirred solution of 2-aminoethanol (468 mg, 7.66 mmol, 1.0 eq) in acetonitrile (15 mL), was added 9-methyldecyl 8-bromo-2-methyloctanoate (3.0 g, 7.66 mmol, 1.0 eq) and N-ethylbis(isopropyl)amine (2.68 mL, 15.3 mmol, 2 eq.) to it. The reaction was stirred at 65° C. for 5 days. The progress of reaction was monitored by ELSD/TLC (41% product form in reaction mixture by ELSD). The reaction mixture was concentrated under reduced pressure and the crude was purified by flash column chromatography (SiO₂: 0-10% Methanol in Dichloromethane) to give 9-methyldecyl 8-(2-hydroxyethylamino)-2-methyloctanoate (1.1 g, Yield=38.37%) as a white solid. ELSD analysis: Purity 99.02%, Calculated C₂₂H₄₆NO₃, [M+H]=372.34, Observed=372.40 (m/z, M+H⁺).
Step 2: Synthesis of 9-methyldecyl 8-((8-((10-fluoro-2-hexyldecyl)oxy)-8-oxooctyl)(2-hydroxyethyl)amino)-2-methyloctanoate

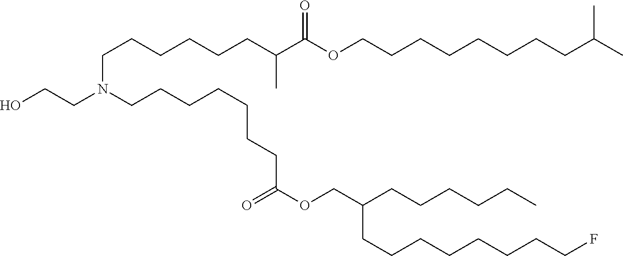 [SMILES: CCCCCCC(CCCCCCCCF)COC(=O)CCCCCCCN(CCO)CCCCCCC(C)C(=O)OCCCCCCCCC(C)C]

To a stirred solution of 10-fluoro-2-hexyldecyl 8-bromooctanoate (501 mg, 1.08 mmol, 1.0 eq) in acetonitrile (10 mL) was added 9-methyldecyl 8-(2-hydroxyethylamino)-2-methyloctanoate (0.4 g, 1.08 mmol 1.0 eq) and N-ethylbis(isopropyl)amine (417 mg, 3.23 mmol, 3 eq). The reaction was stirred at 100° C. for 48 h. The progress of reaction was monitored by ELSD/TLC (76% product form in reaction mixture by ELSD). The reaction mixture was concentrated under reduced pressure and the crude was purified by flash column chromatography (SiO₂: 0-10% Methanol in Dichloromethane) to get 9-methyldecyl 8-{[7-(10-fluoro-2-hexyldecyloxycarbonyl)heptyl](2-hydroxyethyl)amino}-2-methyloctanoate (115 mg) as pale brown liquid. The compound (115 mg) was obtained after column chromatography was dissolved in dichloromethane (10 mL) and added anhydrous potassium carbonate (5 eq) under nitrogen atmosphere and stirred vigorously for 2 h. Then, reaction mixture was filtered through hydrophobic PTFE syringe filter (25 mm×0.45 μm) using glass syringe to get 9-methyldecyl 8-{[7-(10-fluoro-2-hexyldecyloxycarbonyl)heptyl](2-hydroxyethyl)amino}-2-methyloctanoate (101 mg, Yield=12.41%) as a pale yellow liquid. ¹H-NMR (400 MHz, CDCl₃-d₃): δ 4.49 (t, J=6.0 Hz, 1H), 4.37 (t, J=6.0 Hz, 1H), 4.05 (t, J=6.8 Hz, 2H), 3.96 (d, J=6.0 Hz, 2H), 3.75 (b, 2H), 2.93-2.63 (b, 4H), 2.45-2.38 (m, 1H), 2.29 (t, J=7.2 Hz, 2H), 1.73-1.47 (m, 14H), 1.42-1.21 (m, 48H), 1.13 (d, J=7.2 Hz, 3H), 0.89-0.85 (m, 9H). ¹⁹F-NMR (375 MHz, CDCl₃-d₃): −217.99, ELSD analysis: Purity 97.94%, Calculated C₄₆H₉₁FNO₅, [M+H]=756.68, Observed=756.65 (m/z, M+H⁺).
Example 188. Synthesis of Compound 74: 8-((2-hydroxyethyl)(7-methyl-8-(nonyloxy)-8-oxooctyl)amino)octyl 2-butyl-8-fluorooctanoate
Step 1: Synthesis of nonyl 8-((2-hydroxyethyl)amino)-2-methyloctanoate

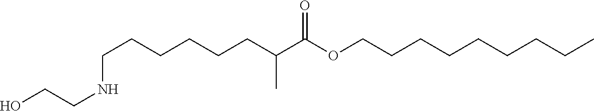 [SMILES: CCCCCCCCCOC(=O)C(C)CCCCCCNCCO]

To a stirred solution of nonyl 8-bromo-2-methyloctanoate (1 g, 2.75 mmol, 1.0 eq) in acetonitrile (10 mL), was added 2-aminoethanol (168 mg, 2.75 mmol, 1.0 eq) and N-ethylbis(isopropyl)amine (1.44 mL, 8.26 mmol, 3 eq) to it. The reaction was stirred at 75° C. for 72 h. The progress of reaction was monitored by ELSD/TLC (41% product form in reaction mixture by ELSD). The reaction mixture was concentrated under reduced pressure and the crude was purified by flash column chromatography (SiO₂: 0-10% Methanol in Dichloromethane) to get nonyl 8-(2-hydroxyethylamino)-2-methyloctanoate (250 mg, Yield=26.55%) as colorless liquid. ELSD analysis: Purity 99.91%, Calculated C₂₀H₄₂NO₃, [M+H]=344.31, Observed=344.40 (m/z, M+H⁺).
Step 2: Synthesis of 8-hydroxyoctyl acetate

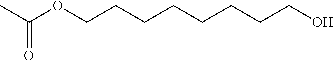

To a stirred solution of 1,8-octanediol (10 g, 68.4 mmol) in tetrahydrofuran (90.4 mL) was added of pyridine (11 mL, 137 mmol, 2 eq) at 0° C. and stirred for 30 min at same temperature. Then acetyl acetate (6.71 mL, 68.4 mmol, 1.0 eq) was added to it and the reaction mixture was stirred at r.t. for 16 h. The progress of reaction was monitored by TLC. The reaction mixture was quenched with ice cooled NaHCO₃ (250 ml) and extracted with ethyl acetate (250×3 mL). The resulting organic layer was dried over Na₂SO₄, and concentrated under reduce pressure, and the crude was purified by flash column chromatography (SiO₂: 0-15% Ethyl acetate in Hexane), to get 8-hydroxyoctyl acetate (6.3 g, Yield=48.93%) as a colorless liquid. ¹H NMR (400 MHz, CDCl₃-d3): δ 4.03 (t, J=6.8 Hz, 2H), 3.61 (t, J=6.8 Hz, 2H), 2.03 (s, 3H), 1.61-1.51 (m, 4H), 1.31 (s, 8H).
Step 3: Synthesis of 8-fluorooctyl acetate

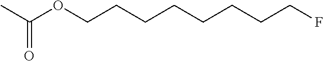

To a stirred solution of 8-hydroxyoctyl acetate (5.4 g, 28.7 mmol, 1.0 eq) in dichloromethane (54 ml), was added dropwise N,N-diethyl(trifluorothio)amine (11.4 mL, 86 mmol, 3.0 eq) at −78° C. The reaction mixture was stirred at r.t. for 4 h. The progress of reaction was monitored by TLC (SM was consumed). The mixture was quenched with sat. aq NaHCO₃, and extracted with DCM (3×50 mL). The resulting organic layer was dried over Na₂SO₄, and concentrated under reduce pressure. The crude was purified by flash column chromatography (SiO₂: 0-4% Ethyl acetate in Hexane), to get 8-fluorooctyl acetate (5.1 g, 93.46%) as a pale-yellow liquid. ¹H NMR (400 MHz, CDCl₃-d3): δ 4.49 (t, J=6.0 Hz, 1H), 4.37 (t, J=6.4 Hz, 1H), 4.04 (t, J=6.8 Hz, 2H), 2.04 (s, 3H), 1.73-1.58 (m, 4H), 1.33 (s, 8H).
Step 4: Synthesis of 8-fluorooctan-1-ol

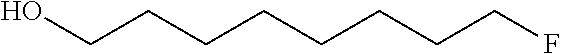

To a stirred solution of 8-fluorooctyl acetate (5.0 g, 26.3 mmol, 1.0 eq) in tetrahydrofuran (100 mL) was added Lithium aluminium hydride 1.0 M in THF (1.5 g, 39.4 mmol, 1.5 eq) dropwise at −78° C. and the reaction mixture was stirred at room temperature for 4 hr. After completion of reaction (monitored by TLC) the reaction mixture was quenched with aq. ammonium chloride solution and extracted with ethyl acetate (3×250 ml) times. The organic layer was dried over sodium sulphate, filtered and concentrated under vacuum to get the crude mixture. The crude product was purified by flash chromatography (SiO₂: 0-15% Ethyl acetate in Hexane), to get 8-fluoro-1-octanol (3.8 g, Yield=97.55%) as a colorless liquid. ¹H NMR (400 MHz, DMSO-d6): δ 4.48 (t, J=6.4 Hz, 1H), 4.38-4.32 (m, 2H), 3.39-3.35 (m, 2H), 1.69-1.56 (m, 2H), 1.42-1.27 (s, 10H).
Step 5: Synthesis of 8-fluorooctanoic acid

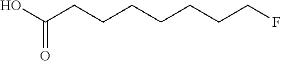 [SMILES: O=C(O)CCCCCCCF]

To a stirred solution of chromamethanetrione-sulfuric acid (25.7 mL, 169 mmol, 5 eq) and water (20 mL) was cooled to −10° C., and a solution od 8-fluoro-1-octanol (5 g, 33.7 mmol, 1 eq) in acetone (60 mL) was added dropwise. The reaction mixture was stirred for overnight at room temperature. After completion of the reaction (monitored by TLC). Reaction mixture was diluted with water (200 mL) and extracted with diethylether (5×100 ml). The resulting organic layer was dried over Na₂SO₄, and concentrated under reduce pressure. The crude was purified by flash column chromatography (SiO₂: 0-25% Ethyl acetate in Hexanes) to get 8-fluorooctanoic acid (2.6 g, Yield=47.52%) as a white crystal. ¹H NMR (400 MHz, DMSO-d6): δ 11.98 (s, 1H), 4.86 (t, J=6.0 Hz, 1H), 4.36 (t, J=6.0 Hz, 1H), 2.19 (t, J=7.2 Hz, 2H), 1.69-1.56 (m, 2H), 1.52-1.47 (m, 2H), 1.32-1.26 (m, 6H).
Step 6: Synthesis of 2-butyl-8-fluorooctanoic acid

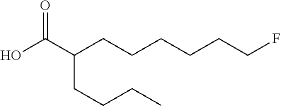

To a stirred solution of 8-fluorooctanoic acid (4.0 g, 24.7 mmol, 1.0 eq.) in tetrahydrofuran (60 mL), was added sodium hydride (1.18 g, 29.6 mmol, 1.2 eq.) at 0° C. and stirred for 30 minutes. Then, lithium bis(isopropyl)azanide (3.43 g, 32.1 mmol, 1.3 eq.) was added to the reaction mixture at the −50° C. The reaction mixture was stirred at room temperature for 30 minutes and added 1-iodobutane (4.54 g, 24.7 mmol, 1.0 eq.). The reaction mixture was stirred at 45° C. for 16 h. The progress of reaction was monitored by TLC. The reaction mixture was poured into 1M HCl (250 ml) and extracted with ethyl acetate (3×250 ml). The resulting organic layer was dried over sodium sulfate, and concentrated under reduce pressure. The crude thus obtained was purified by flash column chromatography (silica gel, 0-2% gradient of ethyl acetate in hexane) to obtain 2-butyl-8-fluorooctanoic acid (1.5 g, Yield=27.86%) as a colorless liquid. ¹H NMR (400 MHz, CDCl₃-d3): δ 11.95-10.15 (b, 1H), 4.51 (t, J=6.4 Hz, 1H), 4.39 (t, J=6.0 Hz, 1H), 2.41-2.34 (m, 1H), 1.77-1.63 (m, 4H), 1.53-1.20 (m, 12H), 0.93 (t, J=6.4 Hz, 3H).
Sep 7: Synthesis of 8-bromooctyl 2-butyl-8-fluorooctanoate

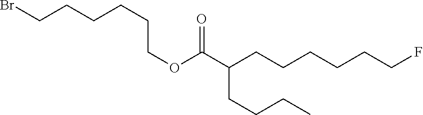 [SMILES: CCCCC(CCCCCCF)C(=O)OCCCCCCBr]

To a stirred solution of 2-butyl-8-fluorooctanoic acid (1.5 g, 6.87 mmol, 1 eq) in dichloromethane (50 mL), was added 4-(dimethylamino)pyridin-1-ium (1.26 g, 10.3 mmol, 1.5 eq) and {3-[cyano(ethyl)amino]propyl}dimethylazanium chloride (3.29 g, 17.2 mmol, 2.5 eq) The reaction mixture was stirred for 15 min and added 8-bromo-1-octanol (1.44 g, 6.87 mmol, 1.0 eq) to it. The reaction mixture was stirred at room temperature for 16 h. The progress of reaction was monitored by TLC (SM was consumed). Water (100 mL) was added to the reaction mixture and extracted with DCM (3×100 mL). The resulting organic layer was dried over Na₂SO₄, and concentrated under reduce pressure, and the crude was purified by flash column chromatography (SiO₂: 0-2% Ethyl acetate in Hexanes) to give 8-bromooctyl 2-butyl-8-fluorooctanoate (1 g, Yield=35.55%) as a colorless liquid. ¹H NMR (400 MHz, CDCl₃-d3): δ 4.48 (t, J=6.0 Hz, 1H), 4.36 (t, J=6.0 Hz, 1H), 4.06 (t, J=6.4 Hz, 2H), 3.54-3.38 (m, 2H), 2.34-2.27 (m, 1H), 1.89-1.82 (m, 2H), 1.74-1.58 (m, 6H), 1.51-1.19 (m, 20H), 0.89-0.86 (m, 3H).
Step 8: Synthesis of 8-((2-hydroxyethyl)(7-methyl-8-(nonyloxy)-8-oxooctyl)amino)octyl 2-butyl-8-fluorooctanoate

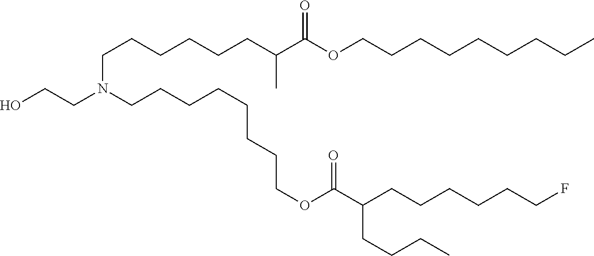 [SMILES: CCCCCCCCCOC(=O)C(C)CCCCCCN(CCO)CCCCCCCCOC(=O)C(CCCC)CCCCCCF]

To a stirred solution of 8-bromooctyl 2-butyl-8-fluorooctanoate (441 mg, 1.08 mmol, 1.0 eq) in acetonitrile (10 mL), was added nonyl 8-(2-hydroxyethylamino)-2-methyloctanoate (370 mg, 1.08 mmol, 1.0 eq) and N-ethylbis(isopropyl)amine (940 μL, 5.38 mmol, 5 eq.) to it. The reaction was stirred at 100° C. for 48 h. The progress of reaction was monitored by ELSD/TLC (88% product form in reaction mixture by ELSD). The reaction mixture was concentrated under reduced pressure and the crude was purified by flash column chromatography (SiO₂: 0-10% Methanol in Dichloromethane) to get nonyl 8-{[8-(1-butyl-7-fluoroheptylcarbonyloxy)octyl](2-hydroxyethyl)amino}-2-methyloctanoate (250 mg) as colorless liquid. The compound (250 mg) was obtained after column chromatography was dissolved in dichloromethane (10 ml) and added anhydrous potassium carbonate (5 eq) under nitrogen atmosphere and stirred vigorously for 2 h. Then, reaction mixture was filtered through hydrophobic PTFE syringe filter (25 mm×0.45 μm) using glass syringe to get nonyl 8-{[8-(1-butyl-7-fluoroheptylcarbonyloxy)octyl](2-hydroxyethyl)amino}-2-methyloctanoate (238 mg, Yield=27.5%) as a colorless liquid. ¹H-NMR (400 MHz, CDCl₃-d3): δ 4.48 (t, J=6.0 Hz, 1H), 4.36 (t, J=6.0 Hz, 1H), 4.07-4.03 (t, J=6.0 Hz, 4H), 3.77 (b, 2H), 2.98-2.60 (b, 4H), 2.43-2.33 (m, 2H), 2.32-2.29 (m, 1H), 1.73-1.55 (m, 14H), 1.47-1.26 (m, 39H), 1.14 (d, J=7.2 Hz, 3H), 0.89-0.866 (m, 6H).
Example 189. Synthesis of Compound 75: 9-methyldecyl 8-((8-((1-fluoroheptadecan-9-yl)oxy)-8-oxooctyl)(3-hydroxy-2-methylpropyl)amino)-2-methyloctanoate
Step 1: Synthesis of 1-fluoroheptadecan-9-yl 8-((3-hydroxy-2-methylpropyl)amino)octanoate

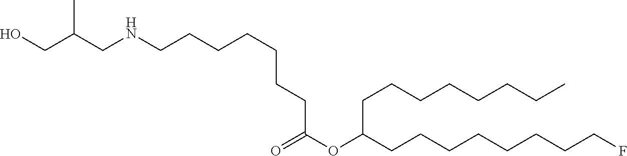 [SMILES: CCCCCCCCC(CCCCCCCCF)OC(=O)CCCCCCCNCC(C)CO]

To a stirred solution of 3-amino-2-methyl-1-propanol (242 mg, 2.71 mmol, 1.0 eq) in acetonitrile (15 mL), was added 9-fluoro-1-octylnonyl 8-bromooctanoate (1.3 g, 2.71 mmol, 1.0 eq) and N-ethylbis(isopropyl)amine (1.42 mL, 8.13 mmol, 3 eq.). The reaction was stirred at 70° C. for 6 days. The progress of reaction was monitored by ELSD/TLC (35% product form in reaction mixture by ELSD). The reaction mixture was concentrated under reduced pressure and the crude was purified by flash column chromatography (SiO₂: 0-10% Methanol in Dichloromethane) to give 9-fluoro-1-octylnonyl 8-(3-hydroxy-2-methylpropylamino)octanoate (280 mg, Yield=21.18%) as a colorless liquid. ELSD analysis: Purity 99.83%, Calculated C₂₉H₅₉FNO₃, [M+H]=488.44, Observed=488.45 (m/z, M+H⁺).
Step 2: Synthesis of 9-methyldecyl 8-((8-((1-fluoroheptadecan-9-yl)oxy)-8-oxooctyl)(3-hydroxy-2-methylpropyl)amino)-2-methyloctanoate

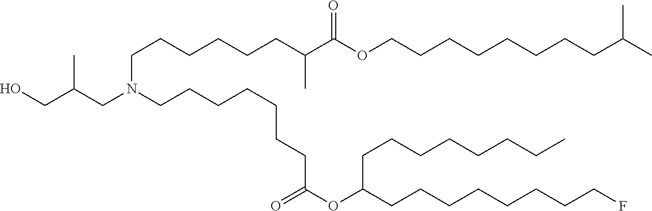 [SMILES: CCCCCCCCC(CCCCCCCCF)OC(=O)CCCCCCCN(CCCCCCC(C)C(=O)OCCCCCCCCC(C)C)CC(C)CO]

To a stirred solution of compound 9-fluoro-1-octylnonyl 8-(3-hydroxy-2-methylpropylamino)octanoate (0.5 g, 1.03 mmol, 1.0 eq) in acetonitrile (10 mL) was added N-ethylbis(isopropyl)amine (397 mg, 3.08 mmol, 3 eq) and 9-methyldecyl 8-bromo-2-methyloctanoate (410 mg, 1.03 mmol, 1.0 eq). The reaction mixture was stirred at 100° C. for 4 days. The progress of reaction was monitored by TLC/ELSD. The reaction mixture was concentrated then quenched with saturated sodium bicarbonate solution (50 ml) and extracted with dichloromethane (50 ml×3). The resulting organic layer was dried over sodium sulphate, and concentrated under reduce pressure to get crude. The crude was purified by flash column chromatography (SiO₂: 0-6% methanol in dichloromethane), to give 9-methyldecyl 8-{[7-(9-fluoro-1-octylnonyloxycarbonyl)heptyl](3-hydroxy-2-methylpropyl)amino}-2-methyloctanoate (98 mg, Yield=11.98%) as a Colorless liquid. The compound was dissolved in dichloromethane (10 ml) and filtered through Hydrophobic PTFE Syringe Filter, 25 mm*0.45 μm by glass syringe to get desired compound. ¹H-NMR (400 MHz, CDCl₃-d3): δ 4.88-4.82 (m, 1H), 4.48 (t, J=6.0 Hz, 1H), 4.37 (t, J=6.0 Hz, 1H), 4.05 (t, J=6.8 Hz, 2H), 3.70 (b, 1H), 3.48 (t, J=6.0 Hz, 1H), 3.21-2.67 (b, 4H), 2.43-2.38 (m, 1H), 2.27 (t, J=6.0 Hz, 2H), 2.15 (b, 2H), 1.72-1.54 (m, 9H), 1.52-1.47 (m, 8H), 1.42-1.21 (m, 48H), 1.13 (t, J=6.8 Hz, 3H), 0.89-0.85 (m, 9H), 0.75 (b, 2H). ¹⁹F-NMR (375 MHz, CDCl₃-d₃): −217.98 ELSD analysis: Purity 99.86%, Calculated C₄₉H₉₇FNO₅, [M+H]=798.73, Observed=798.70 (m/z, M+H⁺).
Example 190. Synthesis of Compound 76: 8-((4-hydroxybutyl)(7-methyl-8-(nonyloxy)-8-oxooctyl)amino)octyl 2-butyl-8-fluorooctanoate
Step 1: Synthesis of nonyl 8-((4-hydroxybutyl)amino)-2-methyloctanoate

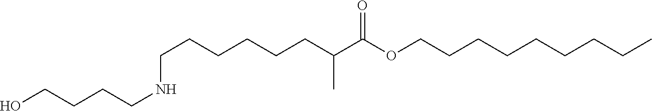 [SMILES: CCCCCCCCCOC(=O)C(C)CCCCCCNCCCCO]

To a stirred solution of nonyl 8-bromo-2-methyloctanoate (1 g, 2.75 mmol, 1.0 eq) in acetonitrile (10 mL), was added 4-amino-1-butanol (245 mg, 2.75 mmol, 1.0 eq) and N-ethylbis(isopropyl)amine (2.4 mL, 13.8 mmol, 5.0 eq) to it. The reaction was stirred at 75° C. for 72 h. The progress of reaction was monitored by ELSD/TLC (46% product form in reaction mixture by ELSD). The reaction mixture was concentrated under reduced pressure and the crude was purified by flash column chromatography (SiO₂: 0-10% Methanol in Dichloromethane) to get nonyl 8-(4-hydroxybutylamino)-2-methyloctanoate (450 mg, Yield=44%) as colorless liquid. ELSD analysis: Purity 99.87%, Calculated C₂₂H₄₆NO₃, [M+H]=372.34, Observed=372.40 (m/z, M+H⁺).
Step 2: Synthesis of 8-((4-hydroxybutyl)(7-methyl-8-(nonyloxy)-8-oxooctyl)amino)octyl 2-butyl-8-fluorooctanoate

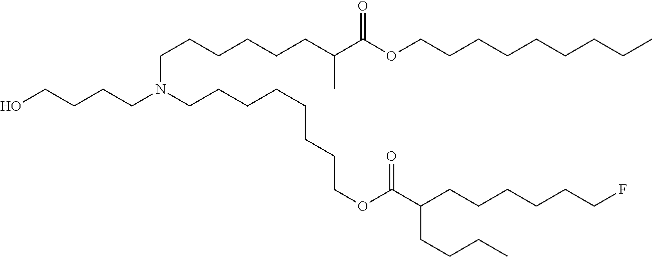 [SMILES: CCCCCCCCCOC(=O)C(C)CCCCCCN(CCCCO)CCCCCCCCOC(=O)C(CCCC)CCCCCCF]

To a stirred solution of 8-bromooctyl 2-butyl-8-fluorooctanoate (441 mg, 1.08 mmol, 1.0 eq) in acetonitrile (10 mL), was added nonyl 8-(4-hydroxybutylamino)-2-methyloctanoate (0.4 g, 1.08 mmol, 1.0 eq) and N-ethylbis(isopropyl)amine (940 μL, 5.38 mmol, 5 eq.). The reaction was stirred at 100° C. for 48 h. The progress of reaction was monitored by ELSD/TLC (70% product form in reaction mixture by ELSD). The reaction mixture was concentrated under reduced pressure and the crude was purified by flash column chromatography (SiO₂: 0-10% Methanol in Dichloromethane) to get nonyl 8-{[8-(1-butyl-7-fluoroheptylcarbonyloxy)octyl](4-hydroxybutyl)amino}-2-methyloctanoate (200 mg) as colorless liquid. The compound (200 mg) was obtained after column chromatography was dissolved in dichloromethane (10 ml) and added anhydrous potassium carbonate (5 eq) under nitrogen atmosphere and stirred vigorously for 2 h. Then, reaction mixture was filtered through hydrophobic PTFE syringe filter (25 mm×0.45 μm) using glass syringe to get nonyl 8-{[8-(1-butyl-7-fluoroheptylcarbonyloxy)octyl](4-hydroxybutyl)amino}-2-methyloctanoate (182 mg, Yield=24%) as a colorless liquid. ¹H-NMR (400 MHz, CDCl₃-d₃): δ 4.48 (t, J=6.0 Hz, 1H), 4.36 (t, J=6.0 Hz, 1H), 4.07-4.03 (t, J=6.0 Hz, 4H), 3.61 (b, 2H), 2.78-2.60 (b, 5H), 2.43-2.37 (m, 1H), 2.34-2.27 (m, 1H), 1.75-1.55 (m, 14H), 1.47-1.26 (m, 44H), 1.13 (d, J=6.8 Hz, 3H), 0.89-0.86 (m, 6H). ¹⁹F-NMR (375 MHz, CDCl₃-d₃): −218.07, ELSD analysis: Purity 99.81%, Calculated C₄₂H₈₃FNO₅, [M+H]=700.62. Observed=700.60 (m/z, M+H⁺).
Example 191. Synthesis of Compound 77:10-fluoro-2-octyldecyl 8-((4-hydroxybutyl)(8-(nonyloxy)-8-oxooctyl)amino)octanoate
Step 1: Synthesis of 10-fluoro-2-octyldecanoic acid

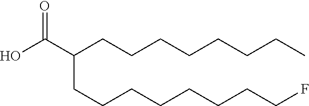 [SMILES: CCCCCCCCC(CCCCCCCCF)C(=O)O]

To a stirred solution of 10-fluorodecanoic acid (1.6 g, 8.41 mmol, 1.0 eq) in tetrahydrofuran (60 mL) was added sodium hydride (404 mg, 10.1 mmol, 1.2 eq) at 0° C. and stirred for 30 minutes. Then, lithium bis(isopropyl)azanide (1.08 g, 1.2 eq. 1.2 eq) was added to the reaction mixture at the −50° C. The reaction mixture was stirred at room temperature for 30 minutes and added 1-iodooctane (2.02 g, 8.41 mmol, 1.0 eq.). The reaction mixture was stirred at 45° C. for 16 h. The progress of reaction was monitored by TLC. The reaction mixture was poured into 1M HCl (250 mL) and extracted with ethyl acetate (3×250 mL). The resulting organic layer was dried over sodium sulfate, and concentrated under reduce pressure. The crude thus obtained was purified by flash column chromatography (silica gel, 0-2% gradient of ethyl acetate in hexane) to obtain 10-fluoro-2-octyldecanoic acid (750 mg, Yield=29.48%) as a yellow liquid. ¹H NMR (400 MHz, CDCl₃-d3): δ 4.49 (t, J=6.4 Hz, 1H), 4.37 (t, J=6.4 Hz, 1H), 2.38-2.34 (m, 1H), 1.72-1.36 (m, 8H), 1.37-1.25 (m, 20H), 0.89-0.85 (m, 3H).
Step 2: Synthesis of 10-fluoro-2-octyldecan-1-ol

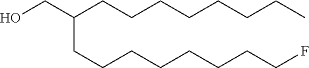 [SMILES: CCCCCCCCC(CO)CCCCCCCCF]

To a stirred solution of 10-fluoro-2-octyldecanoic acid (2 g, 6.61 mmol, 1.0 eq) in tetrahydrofuran (20 mL), was added Lithium aluminium hydride 1.0 M in THF (502 mg, 13.2 mmol, 2.0 eq) dropwise at −78° C. and the reaction mixture was stirred at room temperature for 3 hr. After completion of reaction (monitored by TLC). The reaction mixture was quenched with aq. ammonium chloride solution and extracted with ethyl acetate (3×50 mL). The organic layer was collected and dried over sodium sulphate, filtered and concentrated under vacuum to get the crude mixture. The crude product was purified through combi-flash chromatography (SiO₂: 0-10% Ethyl acetate in Hexane), to get 10-fluoro-2-octyl-1-decanol (1.2 g, Yield=62.91%) as a colorless liquid. ¹H NMR (400 MHz, DMSO-d6): δ 4.47 (t, J=6.0 Hz, 1H), 4.35 (t, J=6.0 Hz, 1H), 4.26 (t, J=5.2 Hz, 1H), 3.26 (t, J=4.8 Hz, 2H), 1.68-1.55 (m, 2H), 1.28-1.13 (s, 27H), 0.85 (t, J=6.4 Hz, 3H).
Step 3: Synthesis of 10-fluoro-2-octyldecyl 8-bromooctanoate

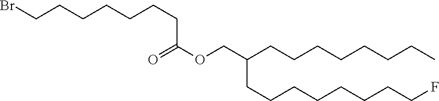 [SMILES: CCCCCCCCC(CCCCCCCCF)COC(=O)CCCCCCCBr]

To a stirred solution of 8-bromooctanoic acid (1.39 g, 6.24 mmol, 1.5 eq) in dichloromethane (30 ml) was added 4-(dimethylamino)pyridin-1-ium (762 mg, 6.24 mmol, 1.5 eq) and {3-[cyano(ethyl)amino]propyl}dimethylazanium chloride (2.39 g, 12.5 mmol, 3 eq). The reaction mixture was stirred for 15 min, then added 10-fluoro-2-octyl-1-decanol (1.2 g, 4.16 mmol, 1.0 eq). The reaction mixture was stirred at r.t. for 16 h. The progress of reaction was monitored by TLC (SM was consumed). Water (100 mL) was added to the reaction mixture, and extracted with DCM (3×100 mL). The resulting organic layer was dried over sodium sulphate, and concentrated under reduce pressure. The crude was purified by flash column chromatography (SiO₂: 0-20% Ethyl acetate in Hexanes) to get a 10-fluoro-2-octyldecyl 8-bromooctanoate (1.2 g, Yield=58.45%) as a colorless liquid. ¹H-NMR (400 MHz, CDCl₃-d3): δ 4.49 (t, J=6.0 Hz, 1H), 4.37 (t, J=6.0 Hz, 1H), 3.97 (d, J=5.6 Hz, 2H), 3.40 (t, J=6.8 Hz, 2H), 2.30 (t, J=7.2 Hz, 2H), 1.88-1.81 (m, 2H), 1.74-1.62 (m, 5H), 1.43-1.27 (m, 32H), 0.89-0.86 (m, 3H).
Step 4: Synthesis of 10-fluoro-2-octyldecyl 8-((4-hydroxybutyl)(8-(nonyloxy)-8-oxooctyl)amino)octanoate

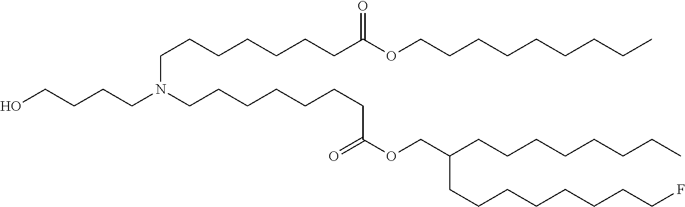 [SMILES: CCCCCCCCCOC(=O)CCCCCCCN(CCCCO)CCCCCCCC(=O)OCC(CCCCCCCC)CCCCCCCCF]

To a stirred solution of nonyl 8-(4-hydroxybutylamino)octanoate (0.4 g, 1.12 mmol, 1.0 eq) in acetonitrile (10 mL) was added 10-fluoro-2-octyldecyl 8-bromooctanoate (497 mg, 1.01 mmol, 0.9 eq) and N-ethylbis(isopropyl)amine (797 μL, 4.47 mmol, 4 eq). The reaction was stirred at 95° C. for 24 h. The progress of reaction was monitored by ELSD/TLC (64% product form in reaction mixture by ELSD). After completion the reaction, reaction mixture was concentrated under reduced pressure and diluted with water (50 mL) and extracted with DCM (2×40 mL). The organic layer collected and again washed with aq. sodium bicarbonate solution (2×50 mL). The organic layer was dried over sodium sulphate, filtered and concentrated to get crude. The crude was purified by flash column chromatography (SiO₂: 0-10% Methanol in Dichloromethane) to get 10-fluoro-2-octyldecyl 8-((4-hydroxybutyl)(8-(nonyloxy)-8-oxooctyl)amino)octanoate (300 mg) as pale yellow liquid. The compound (300 mg) was obtained after column chromatography was dissolved in dichloromethane (10 mL) and was filtered through hydrophobic PTFE syringe filter (25 mm×0.45 μm) using glass syringe to get 10-fluoro-2-octyldecyl 8-((4-hydroxybutyl)(8-(nonyloxy)-8-oxooctyl)amino)octanoate (290, Yield=33.66%) as a pale yellow liquid. ¹H-NMR (400 MHz, CDCl₃-d₃): δ 4.49 (t, J=6.0 Hz, 1H), 4.37 (t, J=6.0 Hz, 1H), 4.05 (t, J=6.8 Hz, 2H), 3.96 (d, J=5.6 Hz, 2H), 3.60 (b, 2H), 2.62 (b, 5H), 2.30-2.26 (m, 4H), 1.74-1.59 (m, 18H), 1.38-1.26 (m, 51H), 0.89-0.85 (m, 6H). ¹⁹F-NMR (375 MHz, CDCl₃-d₃): −217.98, ELSD analysis: Purity 98.53%, Calculated C₄₇H₉₃FNO₅, [M+H⁺]=770.70, Observed=770.65 (m/z, M+H⁺).
Example 192. Synthesis of Compound 78: nonyl 8-((8-((10-fluoro-2-hexyldecyl)oxy)-8-oxooctyl)(2-hydroxyethyl)amino)-2-methyloctanoate

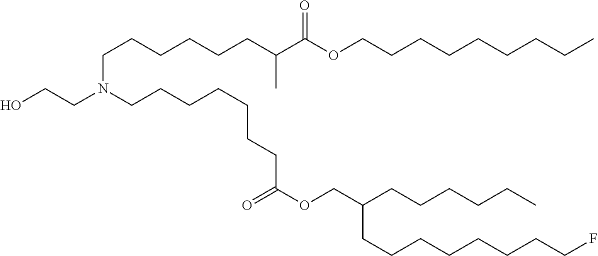 [SMILES: CCCCCCCCCOC(=O)C(C)CCCCCCN(CCO)CCCCCCCC(=O)OCC(CCCCCC)CCCCCCCCF]

To a stirred solution of nonyl 8-(2-hydroxyethylamino)-2-methyloctanoate (0.5 g, 1.46 mmol, 1.0 eq) in acetonitrile (10 mL), was added 10-fluoro-2-hexyldecyl 8-bromooctanoate (678 mg, 1.46 mmol, 1.0 eq) and N-ethylbis(isopropyl)amine (777 μL, 4.37 mmol, 3 eq). The reaction was stirred at 95° C. for 24 h. The progress of reaction was monitored by ELSD/TLC (80% product form in reaction mixture by ELSD). After completion the reaction, reaction mixture was concentrated under reduced pressure and diluted with water (50 mL) and extracted with DCM (2×40 mL). The organic layer collected and again washed with aq. sodium bicarbonate solution (2×50 mL). The organic layer dried over sodium sulphate, filtered and concentrated to get crude. The crude was purified by flash column chromatography (SiO₂: 0-10% Methanol in Dichloromethane) to get nonyl 8-{[7-(10-fluoro-2-hexyldecyloxycarbonyl)heptyl](2-hydroxyethyl)amino}-2-methyloctanoate (253 mg) as pale yellow liquid. The compound (165 mg) was obtained after column chromatography was dissolved in dichloromethane (10 mL) and was filtered through hydrophobic PTFE syringe filter (25 mm×0.45 μm) using glass syringe to get nonyl 8-{[7-(10-fluoro-2-hexyldecyloxycarbonyl)heptyl](2-hydroxyethyl)amino}-2-methyloctanoate (248 mg, Yield=23.4%) as a pale yellow liquid. ¹H-NMR (400 MHz, CDCl₃-d₃): δ 4.49 (t, J=6.0 Hz, 1H), 4.37 (t, J=6.0 Hz, 1H), 4.05 (t, J=6.8 Hz, 2H), 3.96 (d, J=6.0 Hz, 2H), 3.60 (b, 2H), 2.67 (b, 2H), 2.54 (b, 3H), 2.43-2.36 (m, 1H), 2.29 (t, J=7.6 Hz, 2H), 1.74-1.71 (m, 2H), 1.69-1.59 (m, 6H), 1.50 (m, 4H), 1.26 (m, 49H), 1.13 (d, J=6.0 Hz, 3H), 0.89-0.86 (m, 6H). ¹⁹F-NMR (375 MHz, CDCl₃-d₃): −217.99, ELSD analysis: Purity 98.54%, Calculated C₄₄H₅₇FNO₅, [M+H⁺]=728.65, Observed=728.60 (m/z, M+H⁺).
Example 193. Synthesis of Compound 79: 8-((2-hydroxyethyl)(7-methyl-8-((9-methyldecyl)oxy)-8-oxooctyl)amino)octyl 2-butyl-8-fluorooctanoate

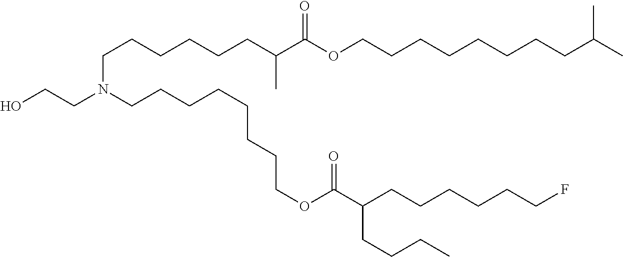 [SMILES: CCCCC(CCCCCCF)C(=O)OCCCCCCCCN(CCO)CCCCCCC(C)C(=O)OCCCCCCCCC(C)C]

To a stirred solution of 8-bromooctyl 2-butyl-8-fluorooctanoate (441 mg, 1.08 mmol, 1.0 eq) in acetonitrile (10 mL) was added 9-methyldecyl 8-(2-hydroxyethylamino)-2-methyloctanoate (0.4 g. 1.08 mmol, 1.0 eq) and N-ethylbis(isopropyl)amine (940 μL, 5.38 mmol, 5 eq.) to it. The reaction was stirred at 100° C. for 48 h. The progress of reaction was monitored by ELSD/TLC (76% product form in reaction mixture by ELSD). The reaction mixture was concentrated under reduced pressure and the crude was purified by flash column chromatography (SiO₂: 0-10% Methanol in Dichloromethane) to get 9-methyldecyl 8-{[8-(1-butyl-7-fluoroheptylcarbonyloxy)octyl](2-hydroxyethyl)amino}-2-methyloctanoate (195 mg) as pale brown liquid. The compound (195 mg) was obtained after column chromatography was dissolved in dichloromethane (10 mL) and added anhydrous potassium carbonate (5 eq) under nitrogen atmosphere and stirred vigorously for 2 h. Then, reaction mixture was filtered through hydrophobic PTFE syringe filter (25 mm×0.45 μm) using glass syringe to get 9-methyldecyl 8-{[8-(1-butyl-7-fluoroheptylcarbonyloxy)octyl](2-hydroxyethyl)amino}-2-methyloctanoate (182 mg, Yield=25.88%) as a pale brown liquid. ¹H-NMR (400 MHz, CDCl₃-d₃): δ 4.48 (t, J=6.0 Hz, 1H), 4.36 (t, J=6.0 Hz, 1H), 4.07-4.03 (t, J=6.0 Hz, 4H), 3.75 (b, 2H), 2.98-2.71 (b, 4H), 2.52-2.38 (m, 2H), 2.34-2.29 (m, 1H), 1.72-1.58 (m, 12H), 1.57-1.21 (m, 43H), 1.13 (d, J=6.8 Hz, 5H), 0.89-0.85 (m, 6H). ¹⁹F-NMR (375 MHz, CDCl₃-d₃): −218.07, ELSD analysis: Purity 98.64%, Calculated C₄₂H₈₃FNO₅, [M+H]=700.62, Observed=700.60 (m/z, M+H⁺).
Example 194. Synthesis of Compound 80: 10-fluoro-2-octyldecyl 8-((2-hydroxyethyl)(8-(nonyloxy)-8-oxooctyl)amino)octanoate

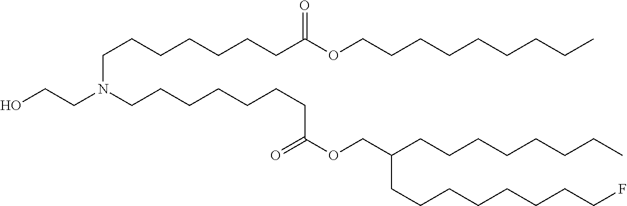 [SMILES: CCCCCCCCCOC(=O)CCCCCCCN(CCO)CCCCCCCC(=O)OCC(CCCCCCCC)CCCCCCCCF]

To a stirred solution of 10-fluoro-2-octyldecyl 8-bromooctanoate (531 mg, 1.08 mmol, 1.0 eq) in acetonitrile (10 mL), was added nonyl 8-(2-hydroxyethylamino)octanoate (355 mg, 1.08 mmol, 1.0 eq) and N-ethylbis(isopropyl)amine (940 μL, 5.38 mmol, 5.0 eq). The reaction was stirred at 100° C. for 24 h. The progress of reaction was monitored by ELSD/TLC (83% product form in reaction mixture by ELSD). After completion the reaction, reaction mixture was concentrated under reduced pressure and diluted with water (50 mL) and extract with DCM (2×40 mL). The organic layer collected and again washed with aq. sodium bicarbonate solution (2×50 mL). The organic layer dried over sodium sulphate, filtered and concentrated to get crude. The crude was purified by flash column chromatography (SiO₂: 0-10% Methanol in Dichloromethane) to get 10-fluoro-2-octyldecyl 8-((2-hydroxyethyl)(8-(nonyloxy)-8-oxooctyl)amino)octanoate (400 mg) as pale yellow liquid. The compound (400 mg) was obtain after column chromatography was dissolved in dichloromethane (10 mL) and was filtered through hydrophobic PTFE syringe filter (25 mm×0.45 μm) using glass syringe to get 10-fluoro-2-octyldecyl 8-((2-hydroxyethyl)(8-(nonyloxy)-8-oxooctyl)amino)octanoate (394 mg, Yield=49.32%) as a pale yellow liquid. ¹H-NMR (400 MHz, CDCl₃-d₃): δ 4.49 (t, J=6.0 Hz, 1H), 4.37 (t, J=6.0 Hz, 1H), 4.05 (t, J=6.8 Hz, 2H), 3.96 (t, J=6.0 Hz, 2H), 3.69 (b, 2H), 2.77-2.59 (b, 5H), 2.31-2.27 (m, 4H), 1.73-1.67 (m, 2H), 1.64-1.57 (m, 10H), 1.44-1.26 (m, 52H), 0.89-0.86 (m, 6H). ¹⁹F-NMR (375 MHz, CDCl₃-d₃): −217.98, ELSD analysis: Purity 98.51%, Calculated C₄₅H₈₉FNO₅, [M+H⁺]=742.66, Observed=742.60 (m/z, M+H⁺).
Example 195. Synthesis of Compound 81: 8-((4-hydroxybutyl)(8-(nonyloxy)-8-oxooctyl)amino)octyl 2-butyl-8-fluorooctanoate

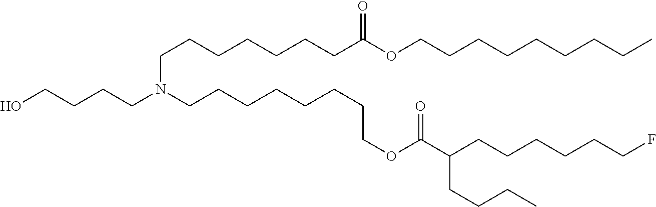 [SMILES: CCCCCCCCCOC(=O)CCCCCCCN(CCCCO)CCCCCCCCOC(=O)C(CCCC)CCCCCCF]

To a stirred solution of nonyl 8-(4-hydroxybutylamino)octanoate (0.4 g, 1.12 mmol, 1.0 eq) in acetonitrile (10 mL), was added 8-bromooctyl 2-butyl-8-fluorooctanoate (412 mg, 1.01 mmol, 0.9 eq) and N-ethylbis(isopropyl)amine (996 μL, 5.59 mmol, 5 eq). The reaction was stirred at 95° C. for 24 h. The progress of reaction was monitored by ELSD/TLC (74% product form in reaction mixture by ELSD). After completion the reaction, reaction mixture was concentrated under reduced pressure and diluted with water (50 mL) and extracted with DCM (2×40 mL). The organic layer collected and again washed with aq. sodium bicarbonate solution (2×50 mL). The organic layer dried over sodium sulphate, filtered and concentrated to get crude. The crude was purified by flash column chromatography (SiO₂: 0-10% Methanol in Dichloromethane) to get 8-((4-hydroxybutyl)(8-(nonyloxy)-8-oxooctyl)amino)octyl 2-butyl-8-fluorooctanoate (165 mg) as pale yellow liquid. The compound (165 mg) was obtained after column chromatography was dissolved in dichloromethane (10 mL) and was filtered through hydrophobic PTFE syringe filter (25 mm×0.45 μm) using glass syringe to get 8-((4-hydroxybutyl)(8-(nonyloxy)-8-oxooctyl)amino)octyl 2-butyl-8-fluorooctanoate (157 mg, Yield=20.53%) as a pale yellow liquid. ¹H-NMR (400 MHz, CDCl₃-d₃): δ 4.48 (t, J=6.0 Hz, 1H), 4.36 (t, J=6.0 Hz, 1H), 4.07-4.03 (m, 4H), 3.56 (b, 2H), 2.49 (b, 6H), 2.34-2.23 (m, 4H), 1.77-1.64 (m, 13H), 1.54-1.18 (m, 43H), 0.89-0.85 (m, 6H). ¹⁹F-NMR (375 MHz, CDCl₃-d₃): −218.066, ELSD analysis: Purity 99.91%, Calculated C₄₁H₈₁FNO₅, [M+H⁺]=686.60, Observed=686.55 (m/z, M+H⁺).
Example 196. Synthesis of Compound 82: 8-((2-hydroxyethyl)(8-(nonyloxy)-8-oxooctyl)amino)octyl 2-butyl-8-fluorooctanoate

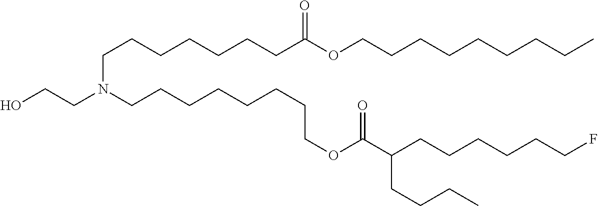 [SMILES: CCCCCCCCCOC(=O)CCCCCCCN(CCO)CCCCCCCCOC(=O)C(CCCC)CCCCCCF]

To a stirred solution of nonyl 8-(2-hydroxyethylamino)octanoate (350 mg, 1.06 mmol, 1.0 eq) in acetonitrile (10 mL), was added 8-bromooctyl 2-butyl-8-fluorooctanoate (391 mg, 956 μmol 0.9 eq) and N-ethylbis(isopropyl)amine (567 μL, 3.19 mmol, 3 eq). The reaction was stirred at 95° C. for 24 h. The progress of reaction was monitored by ELSD/TLC (75% product form in reaction mixture by ELSD). After completion the reaction, reaction mixture was concentrated under reduced pressure and diluted with water (50 mL) and extracted with DCM (2×40 mL). The organic layer collected and again washed with aq. sodium bicarbonate solution (2×50 mL). The organic layer dried over sodium sulphate, filtered and concentrated to get crude. The crude was purified by flash column chromatography (SiO₂: 0-10% Methanol in Dichloromethane) to get 8-{(2-hydroxyethyl)[7-(nonyloxycarbonyl)heptyl]amino}octyl 2-butyl-8-fluorooctanoate (160 mg) as colourless liquid. The compound (160 mg) was obtained after column chromatography was dissolved in dichloromethane (10 mL) and was filtered through hydrophobic PTFE syringe filter (25 mm×0.45 μm) using glass syringe to get 8-{(2-hydroxyethyl)[7-(nonyloxycarbonyl)heptyl]amino}octyl 2-butyl-8-fluorooctanoate (157 mg, Yield=21.89%) as a colourless liquid. ¹H-NMR (400 MHz, CDCl₃-d₃): δ 4.48 (t, J=6.0 Hz, 1H), 4.36 (t, J=6.0 Hz, 1H), 4.07-4.03 (m, 4H), 3.69 (b, 2H), 2.77-2.65 (m, 5H), 2.46-2.26 (m, 4H), 1.70-1.59 (m, 14H), 1.45-1.18 (m, 39H), 0.89-0.85 (m, 6H). ¹⁹F-NMR (375 MHz, CDCl₃-d₃): −218.073, ELSD analysis: Purity 99.78%, Calculated C₃₉H₇₇FNO₅, [M+H⁺]=658.57, Observed=658.55 (m/z, M+H⁺).
Example 197 Synthesis Compound 83:1-chloroheptadecan-9-yl 8-((2-hydroxyethyl)(8-(nonyloxy)-8-oxooctyl)amino)octanoate
Step 1: Synthesis of 1-(benzyloxy)heptadecan-9-ol

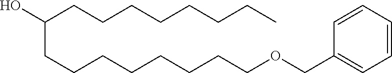 [SMILES: CCCCCCCCC(O)CCCCCCCCOCc1ccccc1]

To a stirred solution of 9-(benzyloxy)nonanal (14 g, 56.4 mmol, 1.0 eq) in tetrahydrofuran (200 ml), was added Octyl magnesium bromide (24.5 g, 113 mmol, 2 eq)(1M soln in diethyl ether) dropwise at −78° C. The reaction mixture was stirred at room temperature for 1 hr, then reaction mixture heated up to 55° C. for 1 hr. After completion of reaction (monitored by TLC), the reaction mixture was quenched with aq. ammonium chloride solution and extracted with ethyl acetate (500 mL×3). The organic layer was collected and dried over sodium sulphate, filtered and concentrated under vacuum to get the crude mixture. The crude product was purified through combi-flash chromatography (SiO₂: 0-15% Ethyl acetate in Hexane) to get 1-(benzyloxy)-9-heptadecanol (6.1 g. Yield=29.84%) as a yellow liquid. ¹H-NMR (400 MHz, DMSO-d6): δ 7.36-7.52 (m, 5H), 4.43 (m, 2H), 4.20 (d, J=5.2 Hz, 1H), 3.40 (t, J=6.8 Hz, 2H), 1.55-1.48 (m, 2H), 1.31-1.23 (m, 27H), 0.86-0.83 (m, 3H).
Step 2: Synthesis of 1-(benzyloxy)heptadecan-9-yl 8-bromooctanoate

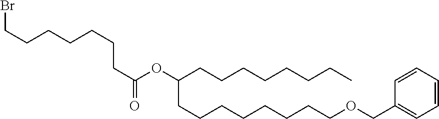 [SMILES: CCCCCCCCC(CCCCCCCCOCc1ccccc1)OC(=O)CCCCCCCBr]

To a stirred solution of 8-bromooctanoic acid (4.5 g, 20.2 mmol, 1.0 eq) in dichloromethane (100 ml), was added 4-(dimethylamino)pyridin-1-ium (3.7 g, 30.3 mmol, 1.5 eq) and {3-[cyano(ethyl)amino]propyl}dimethylazanium chloride (11.6 g, 60.5 mmol, 3 eq). The reaction mixture was stirred for 15 min, then added 1-(benzyloxy)-9-heptadecanol (7.31 g, 20.2 mmol, 1.0 eq). The reaction mixture was stirred at r.t. for 4 h. The progress of reaction was monitored by TLC (SM was consumed). Water (200 mL) was added to the reaction mixture, and extracted with DCM (3×200 mL). The resulting organic layer was dried over sodium sulphate, and concentrated under reduce pressure. The crude was purified by flash column chromatography (SiO₂: 0-20% Ethyl acetate in Hexanes), to get a 9-(benzyloxy)-1-octylnonyl 8-bromooctanoate (9 g, Yield=78.6%) as a colorless liquid. ¹H-NMR (400 MHz, CDCl₃-d3): δ 7.36-7.27 (t, J=6.0 Hz, 5H), 4.89-4.83 (m, 1H), 4.50 (s, 2H), 3.43 (dt, J=24.2, 6.8 Hz, 4H), 2.32-2.26 (m, 2H), 1.88-1.81 (m, 2H), 1.63-1.58 (m, 4H), 1.50-1.41 (m, 6H), 1.36-1.25 (m, 26H), 0.89-0.86 (m, 3H).
Step 3: Synthesis of 2-((tert-butyldimethylsilyl)oxy)ethan-1-amine.

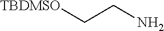 [SMILES: CC(C)(C)[Si](C)(C)OCCN]

To a stirred solution of 2-aminoethan-1-ol (10 g, 164 mmol, 1.0 eq) in dichloromethane (500 mL) was added 1H-imidazole (11.1 g, 164 mmol, 1.0 eq) at room temperature. The resulting reaction mixture was cooled to 0° C. and tert-butyl(chloro)dimethylsilane (49.3 g, 327 mmol, 2 eq.) was added portion-wise at the same temperature. The reaction mixture was stirred for 16 h at room temperature. Progress of the reaction was monitored by ELSD/TLC. Water (500 mL) was added to the reaction mixture and extracted with DCM (3×500 mL). All organic layers were combined and washed with brine solution (2×500 mL). The resulting organic layer was dried over anhydrous sodium sulphate and concentrated under reduced pressure to obtain crude product which was purified over silica using 10% methanol in dichloromethane to obtain 2-((tert-butyldimethylsilyl)oxy)ethan-1-amine (20 g, 69.67%, Yield) as a color less liquid. ¹H-NMR (400 MHz, CDCl₃-d3): δ 3.62 (t, J=5.2 Hz, 2H), 2.77 (t, J=5.2 Hz, 2H), 0.88 (b, 9H), 0.06 (m, 6H). ELSD analysis: Purity 100%, Calculated C₈H₂₂NOSi, [M+H⁺]=175.14, Observed=176.30 (m/z, M+H⁺).
Step 4: Synthesis of nonyl 8-((2-((tert-butyldimethylsilyl)oxy)ethyl)amino)octanoate

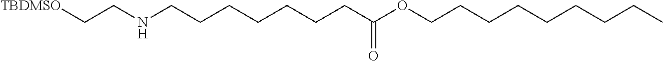 [SMILES: CCCCCCCCCOC(=O)CCCCCCCNCCO[Si](C)(C)C(C)(C)C]

To a stirred solution of 2-[(tert-butyl)bis(methyl)siloxy]ethylamine (2.51 g, 14.3 mmol, 1.0 eq) in acetonitrile (54 mL) was added N-ethylbis(isopropyl)amine (7.5 mL, 42.9 mmol, 3.0 eq) and nonyl 8-bromooctanoate (5 g, 14.3 mmol, 1.0 eq). The reaction mixture was stirred at 55° C. for 3 days. The progress of the reaction was monitored by TLC (SM was consumed). The reaction mixture was concentrated to get the crude mixture. The crude mixture was purified by combi-flash chromatography (SiO₂, 0-20% ethyl acetate in hexane). The desired fractions were collected and evaporated to get nonyl 8-((2-((tert-butyldimethylsilyl)oxy)ethyl)amino)octanoate (2.0 g, 31.49%, Yield) as a yellow liquid. ELSD analysis: Purity 99.89%, Calculated C₂₅H₅₄NO₃Si, [M+H⁺]=444.38, Observed=444.55 (m/z, M+H⁺).
Step 5: Synthesis of 1-(benzyloxy)heptadecan-9-yl 8-((2-((tert-butyldimethylsilyl)oxy)ethyl)(8-(nonyloxy)-8-oxooctyl)amino)octanoate

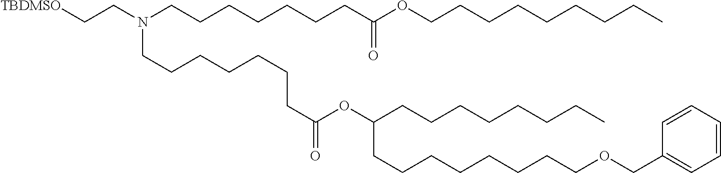 [SMILES: CCCCCCCCCOC(=O)CCCCCCCN(CCCCCCCC(=O)OC(CCCCCCCC)CCCCCCCCOCc1ccccc1)CCO[Si](C)(C)C(C)(C)C]

To a stirred solution of nonyl 8-((2-((tert-butyldimethylsilyl)oxy)ethyl)amino)octanoate (1.5 g, 3.38 mmol, 1.0 eq) in acetonitrile (30 mL), was added 9-(benzyloxy)-1-octylnonyl 8-bromooctanoate (1.92 g, 3.38 mmol, 1.0 eq) and N-ethylbis(isopropyl)amine (3.01 mL, 16.9 mmol, 5 eq). The reaction was stirred at 95° C. for 24 h. The progress of reaction was monitored by ELSD/TLC (SM was consumed). The reaction mixture was concentrated under reduced pressure and the crude was purified by flash column chromatography (SiO₂: 0-20% ethyl acetate in hexane) to get 9-(benzyloxy)-1-octylnonyl 8-({2-[(tert-butyl)bis(methyl)siloxy]ethyl}[7-(nonyloxycarbonyl)heptyl]amino)octanoate (1.5 g, Yield=47.69%) as a colorless liquid. ELSD analysis: Purity 99.78%, Calculated C₅₇H₁₀₈NO₆Si, [M+H⁺]=930.79, Observed=930.75 (m/z, M+H⁺).
Step 6: Synthesis of 1-hydroxyheptadecan-9-yl 8-((2-((tert-butyldimethylsilyl)oxy)ethyl)(8-(nonyloxy)-8-oxooctyl)amino)octanoate

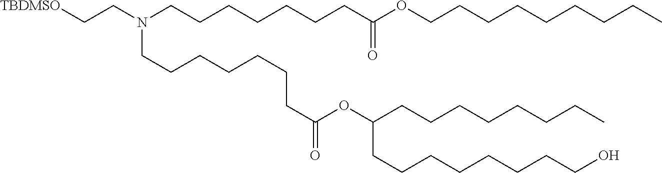 [SMILES: CCCCCCCCCOC(=O)CCCCCCCN(CCCCCCCC(=O)OC(CCCCCCCC)CCCCCCCCO)CCO[Si](C)(C)C(C)(C)C]

To a stirred solution of 9-(benzyloxy)-1-octylnonyl 8-({2-[(tert-butyl)bis(methyl)siloxy]ethyl}[7-(nonyloxycarbonyl)heptyl]amino)octanoate (1 g, 1.07 mmol, 1.0 eq) in methanol (30 mL) and tetrahydrofuran (30 mL) was degassed with nitrogen for 5-10 min, then added Pd/C 10%, 50% wet (1.5 g, w/w). The reaction mixture was stirred at r.t. for 16 h under Hydrogen atmosphere. The progress of reaction was monitored by TLC (SM was consumed). The reaction mixture was filtered through celite bed and washed with mixture of MeOH:THF (3×200 ml). The filtrate was collected and concentrate to get the crude product which was purified through combi-flash chromatography (SiO₂: 0-40% ethyl acetate in hexane) to obtain 1-hydroxyheptadecan-9-yl 8-((2-((tert-butyldimethylsilyl)oxy)ethyl)(8-(nonyloxy)-8-oxooctyl)amino)octanoate (550 mg, Yield=60.9%) as colorless liquid. ¹H NMR (400 MHz, CDCl₃-d3): δ 4.87-4.84 (m, 1H), 4.05 (d, J=6.8 Hz, 2H), 3.67-3.61 (m, 4H), 2.56 (t, J=6.8 Hz, 2H), 2.42 (t, J=7.2 Hz, 4H), 2.30-2.25 (m, 4H), 1.62-1.59 (m, 7H), 1.53-1.44 (m, 6H), 1.42-1.30 (m, 5H), 1.28-1.25 (m, 45H), 0.88-0.86 (m, 15H), 0.05 (s, 6H). ELSD analysis: Purity 99.92%, Calculated C₅₀H₁₀₂NO₆Si, [M+H⁺]=840.74, Observed=840.60 (m/z, M+H⁺).
Step 7: Synthesis of 1-chloroheptadecan-9-yl 8-((2-((tert-butyldimethylsilyl)oxy)ethyl)(8-(nonyloxy)-8-oxooctyl)amino)octanoate

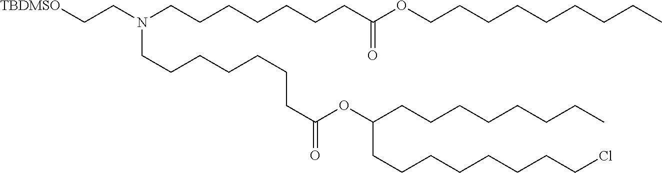 [SMILES: CCCCCCCCCOC(=O)CCCCCCCN(CCCCCCCC(=O)OC(CCCCCCCC)CCCCCCCCCl)CCO[Si](C)(C)C(C)(C)C]

To a stirred solution of 9-hydroxy-1-octylnonyl 8-({2-[(tert-butyl)bis(methyl)siloxy]ethyl}[7-(nonyloxycarbonyl)heptyl]amino)octanoate (400 mg, 476 μmol, 1.0 eq) in dichloromethane (10 mL) was added triethylamine (332 μL, 2.38 mmol, 5.0 eq.). The reaction mixture was stirred for 5 min and added Methanesulfonyl chloride (109 mg, 952 μmol, 2 eq.) at 0° C. The reaction mixture was stirred for 3 h at room temperature. After 3 h, reaction mixture was concentrated under reduced pressure and co-evaporated with toluene under vacuum pressure. The crude was dissolved in dimethylformamide (3 mL) with nitrogen gas purging and added potassium chloride (223 mg, 3.81 μmol, 8.0 eq.) portion wise. The reaction mixture was stirred at 60° C. for 16 h. Progress of reaction mass was monitored by TLC (SM was consumed). The resulting reaction mixture was quenched with cold aqueous sodium bicarbonate solution (20 mL) and extracted with ethyl acetate (3×25 mL). The resulting organic layer was dried over sodium sulphate and concentrated under reduce pressure, to obtain desired crude produced 1-chloroheptadecan-9-yl 8-((2-((tert-butyldimethylsilyl)oxy)ethyl)(8-(nonyloxy)-8-oxooctyl)amino)octanoate (0.250 g, 61.16%, Yield) as colourless liquid, which was used as such in next step. ELSD analysis: Purity 83.51%, Calculated C₅₀H₁₀₁ClNO₅Si=858.71, Observed=858.35 (m/z, M+H⁺).
Step 8: Synthesis of 1-chloroheptadecan-9-yl 8-((2-hydroxyethyl)(8-(nonyloxy)-8-oxooctyl)amino)octanoate

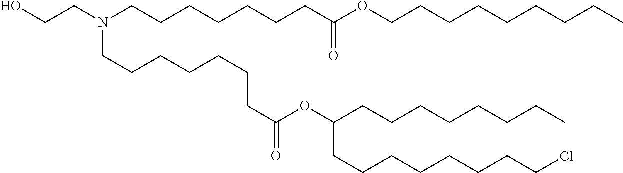 [SMILES: CCCCCCCCCOC(=O)CCCCCCCN(CCO)CCCCCCCC(=O)OC(CCCCCCCC)CCCCCCCCCl]

To a stirred solution of 9-chloro-1-octylnonyl 8-({2-[(tert-butyl)bis(methyl)siloxy]ethyl}[7-(nonyloxycarbonyl)heptyl]amino)octanoate (250 mg, 291 μmol, 1.0 eq) in dichloromethane (4.17 mL) was added 4M HCl in dioxane (53.1 mg, 1.46 mmol, 5 eq) at 0° C. The resulting reaction mixture was stirred for 3 h at room temperature. The progress of reaction mass was monitored by ELSD/TLC (SM was consumed). The resulting reaction mixture was quenched with cold aqueous sodium bicarbonate solution (20 mL) and extracted with ethyl acetate (3×25 mL). The resulting organic layer was dried over sodium sulphate and concentrated under reduce pressure. The crude was purified by flash column chromatography (SiO₂: 0-20% methanol in dichloromethane), to obtain the desired 1-chloroheptadecan-9-yl 8-((2-hydroxyethyl)(8-(nonyloxy)-8-oxooctyl)amino)octanoate (110 mg) as pale yellow liquid. The compound (110 mg) was obtained was dissolved in dichloromethane (10 mL) and was filtered through hydrophobic PTFE syringe filter (25 mm×0.45 μm) using glass syringe to get 1-chloroheptadecan-9-yl 8-((2-hydroxyethyl)(8-(nonyloxy)-8-oxooctyl)amino)octanoate (94 mg, 43.47%, Yield) as a pale yellow liquid. ¹H NMR (400 MHz, CDCl₃-d3): δ 4.88-4.82 (m, 1H), 4.04 (t, J=6.8 Hz, 2H), 3.77 (b, 2H), 3.52 (t, J=6.8 Hz, 2H), 2.89 (b, 2H), 2.78 (b, 4H), 2.30-2.25 (m, 4H), 1.79-1.72 (m, 2H), 1.62-1.59 (m, 9H), 1.50-1.39 (m, 4H), 1.44-1.38 (m, 2H), 1.36-1.25 (m, 46H), 0.89-0.85 (m, 6H). ELSD analysis: Purity 99.94%, Calculated C₄₄H₈₇ClNO₅=744.62, Observed=744.55 (m/z, M+H⁺).
Example 198. Synthesis Compound 84:1,15-difluoropentadecan-8-yl 8-((2-hydroxyethyl)(8-(nonyloxy)-8-oxooctyl)amino)octanoate
Step 1: Synthesis of 1-bromo-7-fluorohentane

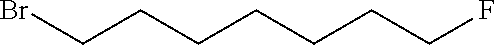

To a stirred solution of 7-bromo-1-heptanol (20 g, 103 mmol, 1.0 eq.) in dichloromethane (350 mL) was cooled to −78° C. and added N,N-diethyl(trifluorothio)amine (24.9 mL, 185 mmol, 1.5 eq.) dropwise. Reaction mixtures was stirred at 25° C. for 16 h. Then reaction mass cooled to −78° C. and added dropwise aqueous sodium bicarbonate solution (100 mL) and extracted with dichloromethane (3×250 mL). The organic layer was collected dried over sodium sulphate, filtered and evaporated under reduced pressure. The crude was purified by flash column chromatography (silica gel, 0-30% gradient of ethyl acetate in hexane) to obtain 1-bromo-7-fluoroheptane (13.1 g, Yield=64.84%) as colorless liquid. ¹H NMR (400 MHz, CDCl₃-d3): δ 4.50 (t, J=6.0 Hz, 1H), 4.38 (t, J=6.0 Hz, 1H), 3.41 (t, J=6.8 Hz, 2H), 1.90-1.83 (m, 2H), 1.77-1.63 (m, 2H), 1.45-1.55 (m, 6H). ¹⁹F-NMR (375 MHz, CDCl₃-d3): δ −218.28.
Step 2: Synthesis of 1,15-difluoropentadecan-8-ol

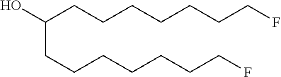 [SMILES: OC(CCCCCCCF)CCCCCCCF]

To a 100 ml RBF attached with condenser, added magnesium (3.34 g, 127 mmol, 5 eq.) and was heated at 80° C. for 1 hour under nitrogen. Cooled the RBF to RT and added tetrahydrofuran (50 mL), iodine (322 mg, 2.54 mmol, 0.1 eq.). Then added 1-bromo-7-fluoroheptane (5 g. 25.4 mmol) at room temperature (dropwise at first, the adding speed can be increased after the mixture became colorless). The reaction mixture was heated for 2 h at 80° C. After 2 h the reaction mixture was cooled to −78° C. and added ethyl formylate (1.04 mL, 0.5 eq., 12.7 mmol) solution in THF. The reaction mixture was allowed to room temperature slowly, and heated to 60° C. for 2 h. The progress of reaction was monitored by TLC. Then reaction mass was cooled to −78° C. and added dropwise a solution of sat. NH₄Cl (50 mL), extracted with ethyl acetate (3×100 mL). The organic layer was collected dried over sodium sulphate, filtered and evaporated under reduced pressure. The crude was purified by flash column chromatography (silica gel, 0-2% ethyl acetate in hexane) to obtain 1,15-difluoro-8-pentadecanol (1.5 g, Yield=22.26%) as colorless liquid. ¹H NMR (400 MHz, CDCl₃-d3): δ 4.49 (t, J=6.0 Hz, 2H), 4.37 (t, J=6.0 Hz, 2H), 3.58 (s, 1H), 1.73-1.64 (m, 4H), 1.43-1.33 (m, 21H). ¹⁹F-NMR (375 MHz, CDCl₃-d3): δ −218.04.

Step 3: Synthesis of 1,15-difluoropentadecan-8-yl 8-bromooctanoate

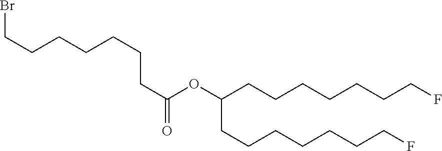 [SMILES: O=C(CCCCCCCBr)OC(CCCCCCCF)CCCCCCCF]

To a stirred solution of 8-bromooctanoic acid (3.29 g, 14.8 mmol, 3.0 eq) in dichloromethane (100 ml) was added 4-(dimethylamino)pyridin-1-ium (901 mg, 7.38 mmol, 1.5 eq) and {3-[cyano(ethyl)amino]propyl}dimethylazanium chloride (2.83 g, 14.8 mmol, 3 eq). The reaction mixture was stirred for 15 min and then added 1,15-difluoro-8-pentadecanol (1.3 g, 4.92 mmol, 1.0 eq) was added. The reaction mixture was stirred at r.t. for 48 h. The progress of reaction was monitored by TLC (SM was consumed). Water (200 mL) was added to the reaction mixture, and extracted with DCM (3×200 mL). The organic layer was dried over sodium sulphate, and concentrated under reduced pressure. The crude was purified by flash column chromatography (SiO₂: 0-2% Ethyl acetate in Hexanes) to get 1,15-difluoropentadecan-8-yl 8-bromooctanoate (1.3 g, Yield=56.32%) as a colorless liquid. ¹H-NMR (400 MHz, CDCl₃-d₃): δ 4.88-4.85 (m, 1H), 4.49 (t, J=6.0 Hz, 2H), 4.37 (t, J=6.0 Hz, 2H), 3.40 (t, J=6.8 Hz, 4H), 2.32-2.26 (m, 2H), 1.88-1.81 (m, 2H), 1.63-1.58 (m, 4H), 1.50-1.41 (m, 4H), 1.36-1.25 (m, 22H).
Step 4: Synthesis of 1,15-difluoropentadecan-8-yl 8-((2-hydroxyethyl)(8-(nonyloxy)-8-oxooctyl)amino)octanoate

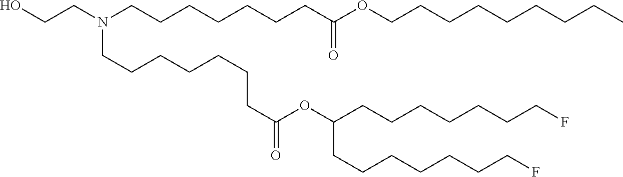 [SMILES: CCCCCCCCCOC(=O)CCCCCCCN(CCO)CCCCCCCC(=O)OC(CCCCCCCF)CCCCCCCF]

To a stirred solution of 8-fluoro-1-(7-fluoroheptyl)octyl 8-bromooctanoate (427 mg, 910 μmol, 1.0 eq) in acetonitrile (10 mL), was added nonyl 8-(2-hydroxyethylamino)octanoate (0.3 g, 910 μmol, 1.0 eq) and N-ethylbis(isopropyl)amine (490 μL, 2.73 mmol, 3 eq) to it. The reaction was stirred at 95° C. for 24 h. The progress of reaction was monitored by ELSD/TLC (71% product form in reaction mixture by ELSD). The reaction mixture was concentrated under reduced pressure. The crude was diluted with water (50 mL) and extracted with DCM (2×40 mL). The organic layer collected and again washed with aq. sodium bicarbonate solution (2×50 mL). The organic layer dried over sodium sulphate, filtered and concentrated to get crude. The crude was purified by flash column chromatography (SiO₂: 0-10% Methanol in Dichloromethane) to get 1,15-difluoropentadecan-8-yl 8-((2-hydroxyethyl)(8-(nonyloxy)-8-oxooctyl)amino)octanoate (250 mg) as pale yellow liquid. The compound (250 mg) was obtained after column chromatography was dissolved in dichloromethane (10 mL) and was filtered through hydrophobic PTFE syringe filter (25 mm×0.45 μm) using glass syringe to get 1,15-difluoropentadecan-8-yl 8-((2-hydroxyethyl)(8-(nonyloxy)-8-oxooctyl)amino)octanoate (237 mg, Yield=36.01%) as a pale yellow liquid. ¹H-NMR (400 MHz, CDCl₃-d₃): δ 4.87-4.84 (m, 1H), 4.48 (t, J=6.0 Hz, 2H), 4.37 (t, J=6.0 Hz, 2H), 4.05 (t, J=6.8 Hz, 2H), 3.55-3.52 (m, 2H), 2.59 (b, 2H), 2.47-2.44 (b, 4H), 2.30-2.25 (m, 4H), 1.74-1.57 (m, 12H), 1.51-1.46 (m, 4H), 1.45-1.40 (m, 4H), 1.36-1.27 (m, 38H), 0.89-0.85 (m, 3H). ¹⁹F-NMR (375 MHz, CDCl₃-d₃): −218.05, ELSD analysis: Purity 99.74%, Calculated C₄₂H₈₂F₂NO₅, [M+H⁺]=718.55, Observed=718.55 (m/z, M+H⁺).
Example 199. Synthesis of Compound 85: nonyl 8-((8-((10-fluoro-2-hexyldecyl)oxy)-8-oxooctyl)(4-hydroxybutyl)amino)-2-methyloctanoate

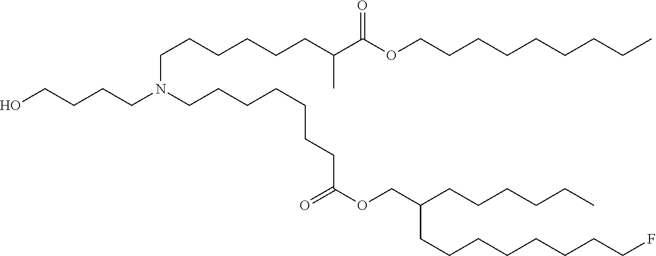 [SMILES: CCCCCCCCCOC(=O)C(C)CCCCCCN(CCCCO)CCCCCCCC(=O)OCC(CCCCCC)CCCCCCCCF]

To a stirred solution of nonyl 8-(4-hydroxybutylamino)-2-methyloctanoate (0.4 g, 1.08 mmol, 1.0 eq) in acetonitrile (10 mL), was added 10-fluoro-2-hexyldecyl 8-bromooctanoate (501 mg, 1.08 mmol, 1.0 eq) and N-ethylbis(isopropyl)amine (958 μL, 5.38 mmol, 3 eq). The reaction was stirred at 95° C. for 24 h. The progress of reaction was monitored by ELSD/TLC (42% product form in reaction mixture by ELSD). After completion the reaction, reaction mixture was concentrated under reduced pressure and diluted with water (50 mL) and extracted with DCM (2×40 mL). The organic layer collected and again washed with aq. sodium bicarbonate solution (2×50 mL). The organic layer dried over sodium sulphate, filtered and concentrated to get crude. The crude was purified by flash column chromatography (SiO₂: 0-10% Methanol in Dichloromethane) to get nonyl 8-((8-((10-fluoro-2-hexyldecyl)oxy)-8-oxooctyl)(4-hydroxybutyl)amino)-2-methyloctanoate (130 mg) as colourless liquid. The compound (130 mg) was obtained after column chromatography was dissolved in dichloromethane (10 mL) and was filtered through hydrophobic PTFE syringe filter (25 mm×0.45 μm) using glass syringe to get nonyl 8-((8-((10-fluoro-2-hexyldecyl)oxy)-8-oxooctyl)(4-hydroxybutyl)amino)-2-methyloctanoate (118 mg, Yield=14.65%) as a pale brown liquid. ¹H-NMR (400 MHz, CDCl₃-d₃): δ 4.49 (t, J=6.0 Hz, 1H), 4.37 (t, J=6.0 Hz, 1H), 4.05 (t, J=6.4 Hz, 2H), 3.96 (d, J=6.0 Hz, 2H), 3.54 (b, 2H), 2.44-2.37 (m, 7H), 2.29 (t, J=7.6 Hz, 2H), 1.73-1.59 (m, 13H), 1.46-1.40 (m, 4H), 1.36-1.27 (m, 47H), 1.13 (m, 3H), 0.89-0.85 (m, 6H). ¹⁹F-NMR (375 MHz, CDCl₃-d₃): −218.00, ELSD analysis: Purity 98.97%, Calculated C₄₆H₉₁FNO₅, [M+H⁺]=756.68, Observed=756.60 (m/z, M+H⁺)
Example 200. Synthesis of Compound 86: 9-methyldecyl 8-((8-((10-fluoro-2-hexyldecyl)oxy)-8-oxooctyl)(4-hydroxybutyl)amino)-2-methyloctanoate
Step 1: Synthesis of 9-methyldecyl 8-((4-hydroxybutyl)amino)-2-methyloctanoate

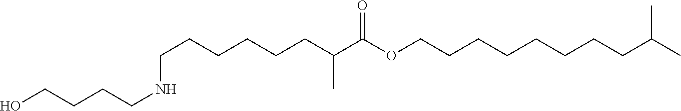 [SMILES: CC(C)CCCCCCCCOC(=O)C(C)CCCCCCNCCCCO]

To a stirred solution of 9-methyldecyl 8-bromo-2-methyloctanoate (3 g, 7.66 mmol, 1.0 eq) in acetonitrile (30 mL, 574 mmol), was added N-ethylbis(isopropyl)amine (6.69 mL, 38.3 mmol, 5.0 eq) and 4-amino-1-butanol (683 mg, 7.66 mmol, 1.0 eq) to it. The reaction mixture was stirred at 55° C. for 72 h. The progress of reaction was monitored by TLC/ELSD. The reaction mixture was concentrated to get the crude mixture. The crude mixture was purified by combi-flash chromatography (SiO₂, 0-10% Methanol in DCM). The desired fractions were collected and evaporated to get 9-methyldecyl 8-(4-hydroxybutylamino)-2-methyloctanoate (0.5 g, Yield=16.32%) as a colorless liquid. ELSD analysis: Purity 98.75%, Calculated: C₂₄H₅₀NO₃, [M+H⁺]=400.37, Observed=400.25 (m/z, M+H+).
Step 2: Synthesis of 9-methyldecyl 8-((8-((10-fluoro-2-hexyldecyl)oxy)-8-oxooctyl)(4-hydroxybutyl)amino)-2-methyloctanoate

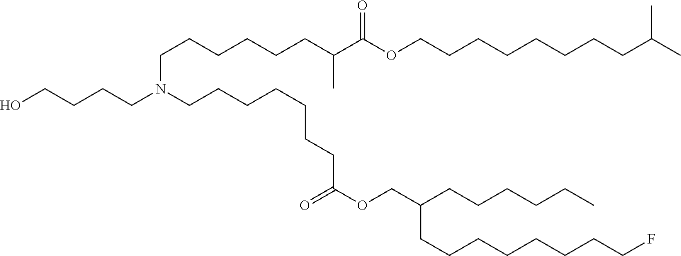 [SMILES: CCCCCCC(CCCCCCCCF)COC(=O)CCCCCCCN(CCCCO)CCCCCCC(C)C(=O)OCCCCCCCCC(C)C]

To a stirred solution of 9-methyldecyl 8-(4-hydroxybutylamino)-2-methyloctanoate (0.4 g, 1 mmol, 1.0 eq) in acetonitrile (5 mL), was added 10-fluoro-2-hexyldecyl 8-bromooctanoate (466 mg, 1 mmol, 1.0 eq) and N-ethylbis(isopropyl)amine (713 μL, 4.0 mmol, 4 eq). The reaction was stirred at 95° C. for 24 h. The progress of reaction was monitored by ELSD/TLC (44% product form in reaction mixture by ELSD). After completion of the reaction, reaction mixture was concentrated under reduced pressure and diluted with water (50 mL) and extracted with DCM (2×40 mL). The organic layer collected and again washed with aq. sodium bicarbonate solution (2×50 mL). The organic layer dried over sodium sulphate, filtered and concentrated to get crude. The crude was purified by flash column chromatography (SiO₂: 0-10% Methanol in Dichloromethane) to get 9-methyldecyl 8-((8-((10-fluoro-2-hexyldecyl)oxy)-8-oxooctyl)(4-hydroxybutyl)amino)-2-methyloctanoate (135 mg) as pale brown liquid. The compound (135 mg) was obtained after column chromatography was dissolved in dichloromethane (10 mL) and was filtered through hydrophobic PTFE syringe filter (25 mm×0.45 μm) using glass syringe to get 9-methyldecyl 8-((8-((10-fluoro-2-hexyldecyl)oxy)-8-oxooctyl)(4-hydroxybutyl)amino)-2-methyloctanoate (133 mg, Yield=16.94%) as a pale brown liquid. ¹H-NMR (400 MHz, CDCl₃-d₃): δ 4.49 (t, J=6.0 Hz, 1H), 4.37 (t, J=6.0 Hz, 1H), 4.05 (t, J=6.4 Hz, 2H), 3.96 (d, J=6.0 Hz, 2H), 3.55 (b, 2H), 2.46-2.36 (m, 7H), 2.30-2.25 (m, 2H), 1.73-1.59 (m, 12H), 1.54-1.47 (m, 5H), 1.40-1.20 (m, 46H), 1.15-1.12 (m, 5H), 0.89-0.85 (m, 9H). ¹⁹F-NMR (375 MHz, CDCl₃-d₃): −218.00, ELSD analysis: Purity 99.75%, Calculated C₄₈H₉₅FNO₅, [M+H⁺]=784.71, Observed=784.45 (m/z, M+H⁺).
Example 201. Synthesis of Compound 87: 7-((7-((10-fluoro-2-octyldecanoyl)oxy)heptyl)(2-hydroxyethyl)amino)heptyl 2,10-dimethylundecanoate
Step 1: Synthesis of 2,10-dimethylundecanoic acid

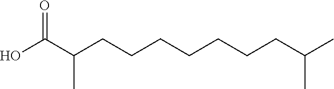

To a stirred solution of 10-methylundecanoic acid (3.0 g, 15.0 mmol, 1.0 eq.) in tetrahydrofuran (60 mL), was added sodium hydride (719 mg, 18.0 mmol, 1.2 eq.) at 0° C. and stirred for 30 minutes. Then added lithium bis(isopropyl)azanide (1.93 g, 18.0 mmol, 1.2 eq.) to the reaction mixture at the −50° C. The reaction mixture was stirred at room temperature for 30 minutes and added iodomethane (1.12 mL, 18.0 mmol, 1.2 eq). The reaction mixture was stirred at 55° C. for 16 h. The progress of reaction was monitored by TLC. The reaction mixture was poured into 1M HCl (250 ml) and extracted with ethyl acetate (3×250 ml). The resulting organic layer was dried over sodium sulfate, and concentrated under reduce pressure. The crude was purified by flash column chromatography (silica gel, 0-2% ethyl acetate in hexane) to obtain 2,10-dimethylundecanoic acid (1.4 g, Yield=43.61%) as a yellow liquid. ¹H NMR (400 MHz, DMSO-d6): δ 11.99 (s, 1H), 2.30-2.25 (m, 1H), 1.55-1.46 (m, 2H), 1.33-1.23 (m, 11H), 1.13-1.12 (b, 2H), 1.03 (d, J=7.2 Hz, 3H). 0.84 (d, J=6.4 Hz, 3H).
Step 2: Synthesis of 7-bromoheptyl 2,10-dimethylundecanoate

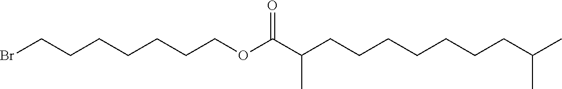 [SMILES: CC(C)CCCCCCCC(C)C(=O)OCCCCCCCBr]

To a stirred solution of 2,10-dimethylundecanoic acid (2 g, 9.33 mmol, 1 eq) in dichloromethane (50 mL), was added 4-(dimethylamino)pyridin-1-ium (1.72 g, 14.0 mmol, 1.5 eq) and {3-[cyano(ethyl)amino]propyl}dimethylazanium chloride (5.37 g, 28 mmol, 3.0 eq). The reaction mixture was stirred for 15 min and added 7-bromo-1-heptanol (1.82 g, 9.33 mmol, 1.0 eq) to it. The reaction mixture was stirred at room temperature for 16 h. The progress of reaction was monitored by TLC (SM was consumed). Water (100 mL) was added to the reaction mixture, and extracted with DCM (3×100 mL). The resulting organic layer was dried over Na₂SO₄, and concentrated under reduce pressure. The crude was purified by flash column chromatography (SiO₂: 0-2% Ethyl acetate in Hexanes) to obtain 7-bromoheptyl 2,10-dimethylundecanoate (1.6 g, Yield=43.81%) as a colorless liquid. ¹H NMR (400 MHz, CDCl₃-d3): δ 4.05 (t, J=6.4 Hz, 2H), 3.40 (t, J=6.8 Hz, 2H), 2.04-2.36 (m, 1H), 1.89-1.77 (m, 2H), 1.66-1.59 (m, 3H), 1.52-1.39 (m, 8H), 1.19-1.18 (b, 10H), 1.12-1.11 (m, 5H), 0.89-0.86 (t, J=6.8 Hz, 6H)
Step 3: Synthesis of 7-((2-hydroxyethyl)amino)heptyl 2,10-dimethylundecanoate

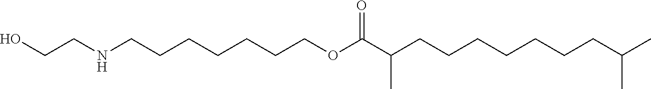 [SMILES: CC(C)CCCCCCCC(C)C(=O)OCCCCCCCNCCO]

To a stirred solution of 7-bromoheptyl 2,10-dimethylundecanoate (1.54 g, 3.93 mmol, 0.8 eq) in acetonitrile (15 mL), was added N-ethylbis(isopropyl)amine (3.43 ml, 19.6 mmol, 4.0 eq) and 2-amino-1-ethanol (0.3 g, 4.91 mmol, 1.0 eq). The reaction mixture was stirred at 55° C. for 72 h. The progress of reaction was monitored by TLC/ELSD (SM was consumed). The reaction mixture was concentrated to get the crude mixture. The crude mixture was purified by combi-flash chromatography (SiO₂, 6-7% Methanol in DCM). The desired fractions were collected and evaporated to get 7-(2-hydroxyethylamino)heptyl 2,10-dimethylundecanoate (680 mg, Yield=37.5%) as a colorless liquid. ELSD analysis: Purity 99.81%, Calculated C₂₂H₄₆NO₃, [M+H⁺]=372.36, Observed=372.20 (m/z, M+H⁺).
Step 4: Synthesis of 7-((7-((10-fluoro-2-octyldecanoyl)oxy)heptyl)(2-hydroxyethyl)amino)heptyl 2,10-dimethylundecanoate

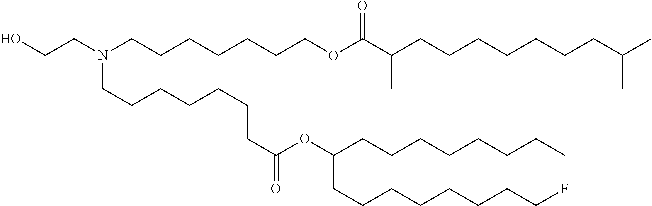 [SMILES: CCCCCCCCC(CCCCCCCCF)OC(=O)CCCCCCCN(CCO)CCCCCCCOC(=O)C(C)CCCCCCCC(C)C]

To a stirred solution of 7-(2-hydroxyethylamino)heptyl 2,10-dimethylundecanoate (0.4 g, 1.08 mmol, 1.0 eq) in acetonitrile (5 mL), was added N-ethylbis(isopropyl)amine (958 μL, 5.38 mmol, 5 eq) and 7-bromoheptyl 10-fluoro-2-octyldecanoate (465 mg, 969 μmol, 0.9 eq.). The reaction was stirred at 95° C. for 24 h. The progress of reaction was monitored by ELSD/TLC (80% product form in reaction mixture by ELSD). The reaction mixture was concentrated under reduced pressure and the crude was purified by flash column chromatography (SiO₂: 0-10% Methanol in Dichloromethane) to get 7-{[7-(9-fluoro-1-octylnonylcarbonyloxy)heptyl](2-hydroxyethyl)amino}heptyl 2,10-dimethylundecanoate (197 mg) as colorless liquid. The compound (197 mg) was obtain after column chromatography was dissolved in dichloromethane (10 mL) and added anhydrous potassium carbonate (5 eq) under nitrogen atmosphere and stirred vigorously for 2 h. Then, reaction mixture was filtered through hydrophobic PTFE syringe filter (25 mm×0.45 μm) using glass syringe to get 7-{[7-(9-fluoro-1-octylnonylcarbonyloxy)heptyl](2-hydroxyethyl)amino}heptyl 2,10-dimethylundecanoate (197 mg, Yield=23.2%) as a colorless liquid. ¹H-NMR (400 MHz, CDCl₃-d₃): δ 4.48 (t, J=6.0 Hz, 1H), 4.36 (t, J=6.0 Hz, 1H), 4.07-4.03 (m, 4H), 3.52 (t, J=5.6 Hz, 2H), 2.56 (t, J=5.2 Hz, 2H), 2.46-2.38 (m, 5H), 2.30-2.29 (m, 1H), 1.72-1.49 (m, 12H), 1.44-1.20 (m, 51H), 1.14-1.12 (m, 3H), 0.89-0.85 (m, 9H). ¹⁹F-NMR (375 MHz, CDCl₃-d₃): −218.00, ELSD analysis: Purity 98.44%, Calculated C₄₇H₉₃NO₅, [M+H⁺]=770.70, Observed=770.60 (m/z, M+H⁺).
Example 202. Synthesis of Compound 88: 8-((4-hydroxybutyl)(7-methyl-8-((9-methyldecyl)oxy)-8-oxooctyl)amino)octyl 2-butyl-8-fluorooctanoate

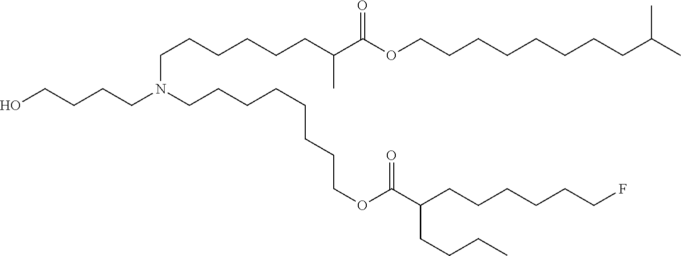 [SMILES: CCCCC(CCCCCCF)C(=O)OCCCCCCCCN(CCCCO)CCCCCCC(C)C(=O)OCCCCCCCCC(C)C]

To a stirred solution of 9-methyldecyl 8-(4-hydroxybutylamino)-2-methyloctanoate (0.4 g, 1 mmol, 1.0 eq) in acetonitrile (5 mL), was added 8-bromooctyl 2-butyl-8-fluorooctanoate (410 mg, 1 mmol, 1.0 eq) and N-ethylbis(isopropyl)amine (891 μL, 5.0 mmol, 5 eq). The reaction was stirred at 95° C. for 24 h. The progress of reaction was monitored by ELSD/TLC (51% product form in reaction mixture by ELSD). After completion the reaction, reaction mixture was concentrated under reduced pressure and diluted with water (50 mL) and extracted with DCM (2×40 mL). The organic layer collected and again washed with aq. sodium bicarbonate solution (2×50 mL). The organic layer dried over sodium sulphate, filtered and concentrated to get crude. The crude was purified by flash column chromatography (SiO₂: 0-10% Methanol in Dichloromethane) to get 8-((4-hydroxybutyl)(7-methyl-8-((9-methyldecyl)oxy)-8-oxooctyl)amino)octyl 2-butyl-8-fluorooctanoate (121 mg) as a pale brown liquid. The compound (121 mg) was obtained after column chromatography was dissolved in dichloromethane (10 mL) and was filtered through hydrophobic PTFE syringe filter (25 mm×0.45 μm) using glass syringe to get 8-((4-hydroxybutyl)(7-methyl-8-((9-methyldecyl)oxy)-8-oxooctyl)amino)octyl 2-butyl-8-fluorooctanoate (116 mg, Yield=15.94%) as a pale brown liquid. ¹H-NMR (400 MHz, CDCl₃-d₃): δ 4.48 (t, J=6.0 Hz, 1H), 4.36 (t, J=6.0 Hz, 1H), 4.07-4.03 (m, 4H), 3.56 (b, 2H), 2.50-2.46 (m, 5H), 2.42-2.39 (m, 1H), 2.30-2.27 (m, 2H), 1.72-1.55 (m, 12H), 1.54-1.47 (m, 5H), 1.40-1.20 (m, 38H), 1.15-1.12 (m, 5H), 0.89-0.85 (m, 9H). ¹⁹F-NMR (375 MHz, CDCl₃-d₃): −218.00, ELSD analysis: Purity 99.65%, Calculated C₄₄H₈₇FNO₅, [M+H⁺]=728.65, Observed=728.40 (m/z, M+H⁺).
Example 203. Synthesis of Compound 89: 7-((7-((10-fluoro-2-octyldecanoyl)oxy)heptyl)(4-hydroxybutyl)amino)heptyl 2,10-dimethylundecanoate
Step 1: Synthesis of 7-((4-hydroxybutyl)amino)heptyl 2,10-dimethylundecanoate

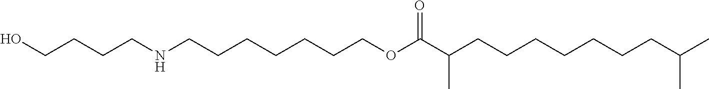 [SMILES: CC(C)CCCCCCCC(C)C(=O)OCCCCCCCNCCCCO]

To a stirred solution of 7-bromoheptyl 2,10-dimethylundecanoate (1.5 g. 3.83 mmol, 1.0 eq) in acetonitrile (15 mL) was added N-ethylbis(isopropyl)amine (2.68 ml, 15.3 mmol, 4.0 eq) and 4-amino-1-butanol (342 mg, 3.83 mmol, 1.0 eq). The reaction mixture was stirred at 55° C. for 48 h. The progress of reaction was monitored by TLC/ELSD. The reaction mixture was concentrated under vacuum to get the crude mixture. The crude mixture was purified by combi-flash chromatography (SiO₂, 6-7% Methanol in DCM). The desired fractions were collected and evaporated to get 7-(4-hydroxybutylamino)heptyl 2,10-dimethylundecanoate (650 mg, Yield=42.44%) as a pale yellow liquid. ELSD analysis: Purity 99.37%, Calculated C₂₄H₅₀NO₃, [M+H⁺]=400.37, Observed=400.25 (m/z, M+H⁺).
Step 2: Synthesis of 7-((7-((10-fluoro-2-octyldecanoyl)oxy)heptyl)(4-hydroxybutyl)amino)heptyl 2,10-dimethylundecanoate

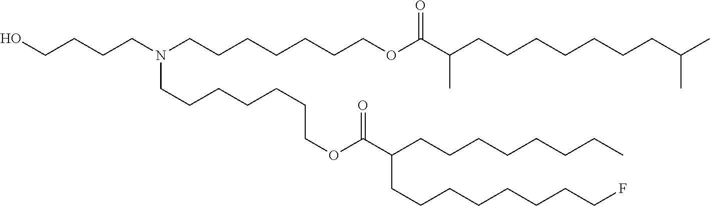 [SMILES: CCCCCCCCC(CCCCCCCCF)C(=O)OCCCCCCCN(CCCCO)CCCCCCCOC(=O)C(C)CCCCCCCC(C)C]

To a stirred solution of 7-(4-hydroxybutylamino)heptyl 2,10-dimethylundecanoate (0.4 g, 1.0 mmol, 1.0 eq) in acetonitrile (5 mL), was added N-ethylbis(isopropyl)amine (713 μL, 4.0 mmol, 4.0 eq) and 7-bromoheptyl 10-fluoro-2-octyldecanoate (480 mg, 1.0 mmol, 1.0 eq.). The reaction was stirred at 95° C. for 24 h. The progress of reaction was monitored by ELSD/TLC (79.97% product form in reaction mixture by ELSD). The reaction mixture was concentrated under reduced pressure and the crude was purified by flash column chromatography (SiO₂: 0-10% Methanol in Dichloromethane) to get 7-((7-((10-fluoro-2-octyldecanoyl)oxy)heptyl)(4-hydroxybutyl)amino)heptyl 2,10-dimethylundecanoate (197 mg) as colorless liquid. The compound (197 mg) was obtain after column chromatography was dissolved in dichloromethane (10 mL) and added anhydrous potassium carbonate (5 eq) under nitrogen atmosphere. The mixture was stirred vigorously for 2 h. Then, reaction mixture was filtered through hydrophobic PTFE syringe filter (25 mm×0.45 μm) using glass syringe to get 7-((7-((10-fluoro-2-octyldecanoyl)oxy)heptyl)(4-hydroxybutyl)amino)heptyl 2,10-dimethyl undecanoate (190 mg, Yield=23.78%) as a pale yellow liquid. ¹H-NMR (400 MHz, CDCl₃-d₃): δ 4.48 (t, J=6.0 Hz, 1H), 4.36 (t, J=6.0 Hz, 1H), 4.07-4.03 (m, 4H), 3.55 (m, 2H), 2.47-2.38 (m, 7H), 2.33-2.28 (m, 1H), 1.72-1.69 (m, 2H), 1.65-1.53 (m, 10H), 1.52-1.45 (m, 6H), 1.44-1.37 (m, 4H), 1.34-1.30 (m, 10H), 1.31-1.20 (m, 32H), 1.13-1.21 (m, 6H), 0.88-0.88 (m, 9H). ¹⁹F-NMR (375 MHz, CDCl₃-d₃): −217.98, ELSD analysis: Purity 99.34%, Calculated C₄₉H₉₇NO₅, [M+H⁺]=798.73, Observed=798.50 (m/z, M+H⁺).

### Example 204. Synthesis of Compound 90

Step 1: Synthesis of 2-(2,6-didodecylmorpholino)ethan-1-ol

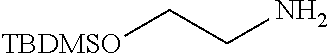 [SMILES: CC(C)(C)[Si](C)(C)OCCN]

To a stirred solution of 2-aminoethan-1-ol (10 g, 164 mmol, 1.0 eq) in dichloromethane (500 mL) was added 1H-imidazole (11.1 g, 164 mmol, 1.0 eq) at room temperature. The resulting reaction mixture was cooled to 0° C. and added tert-butyl(chloro)dimethylsilane (49.3 g. 327 mmol, 2 eq.) portion-wise at the same temperature. The reaction mixture was stirred for 16 h at room temperature. The progress of reaction was monitored by ELSD/TLC. Water (500 mL) was added to the reaction mixture and extracted with DCM (3×500 mL). All organic layers were combined and washed with brine solution (2×500 mL). The resulting organic layer was dried over anhydrous sodium sulphate and concentrated under reduced pressure to obtain crude product which was purified over silica using 10% methanol in dichloromethane to give ((tert-butyldimethylsilyl)oxy)ethan-1-amine (20 g, 69.67%, Yield) as a colourless liquid. ¹H-NMR (400 MHz, CDCl₃-d3): δ 3.62 (t, J=5.2 Hz, 2H), 2.77 (t, J=5.2 Hz, 2H), 0.88 (b, 9H), 0.06 (m, 6H). ELSD analysis: Purity 100%, Calculated C₈H₂₂NOSi, [M+H]=176.14, Observed=176.30 (m/z, M+H⁺).

Step 2: Synthesis of 1,1′-((2-((tert-butyldimethylsilyl)oxy)ethyl)azanediyl)bis(tetradecan-2-ol)

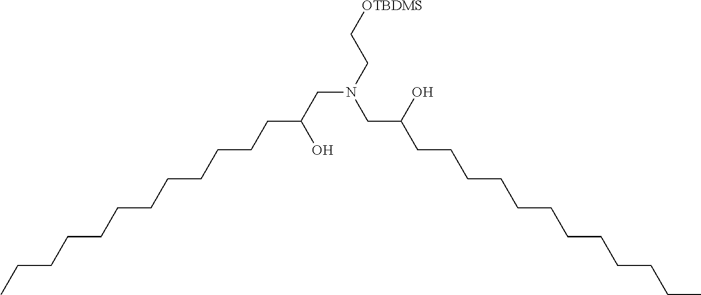 [SMILES: CCCCCCCCCCCCC(O)CN(CCO[Si](C)(C)C(C)(C)C)CC(O)CCCCCCCCCCCC]

A mixture of (2-aminoethoxy)(tert-butyl)dimethylsilane (2 g, 11.4 mmol, 1.0 eq) and 2-dodecyloxirane (6.06 g, 28.5 mmol, 2.5 eq) in isopropanol (25 mL) was heated under nitrogen atmosphere at 95° C. for 16 h. The progress of reaction was monitored by ELSD/TLC (SM was consumed). The reaction mixture was concentrated and the crude was purified by flash column chromatography (SiO₂: 0-20% methanol in dichloromethane) to obtain 1-({2-[(tert-butyldimethylsilyl)oxy]ethyl}(2-hydroxytetradecyl)amino)tetradecan-2-ol (4.6 g, 67.21%, Yield) as yellow liquid. ELSD analysis: Purity 97.02%, Calculated C₃₆H₇₈NO₃Si, [M+H]=600.10, Observed=600.50 (m/z, M+H⁺).
Step 3: Synthesis of 4-(2-((tert-butyldimethylsilyl)oxy)ethyl)-2,6-didodecylmorpholine

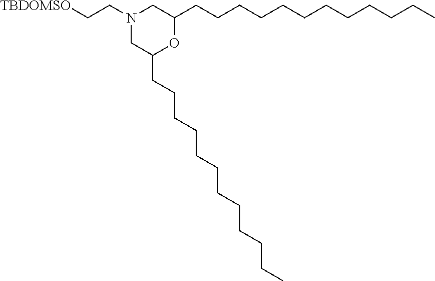

To a stirred solution of 1-({2-[(tert-butyldimethylsilyl)oxy]ethyl}(2-hydroxytetradecyl)amino)tetradecan-2-ol (2.5 g, 4.17 mmol, 1.0 eq) in toluene (25 mL), triphenylphosphane (1.31 g, 5.0 mmol, 1.2 eq) was added portion wise. After 10 minutes N-{[(propan-2-yloxy)carbonyl]imino}(propan-2-yloxy)formamide (1.01 g, 1.2 eq., 5.0 mmol) was added dropwise to the same reaction mixture. The reaction mixture was stirred at 40° C. for 16 hrs. The reaction was monitored by ELSD & TLC. After completion of reaction quenched with water (50 ml) and extracted with ethyl acetate (250 ml). Organic layer was dried over anhydrous sodium sulphate and concentrated under reduced vacuum. The crude was purified by column chromatography (0-10% Ethyl Acetate in Hexane) to give 4-{2-[(tert-butyldimethylsilyl)oxy]ethyl}-2,6-didodecyl morpholine (0.5 g, 20.26%, Yield) as colourless liquid. ELSD analysis: Purity 93.81%, Calculated C₃₆H₇₆NO₂Si, [M+H]=582.56, Observed=582.45 (m/z, M+H⁺).

Step 4: Synthesis of 2-(2,6-didodecylmorpholino)ethan-1-ol

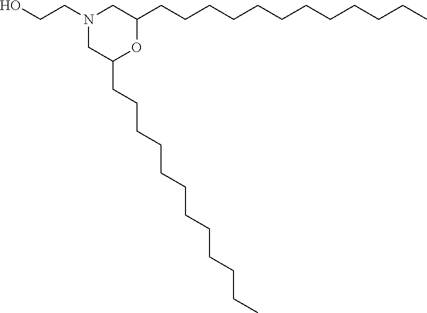 [SMILES: CCCCCCCCCCCCC1CN(CCO)CC(CCCCCCCCCCCC)O1]

To a stirred solution of 4-[2-(tert-butyldimethylsilyl)ethyl]-2,6-didodecylmorpholine (0.4 g, 707 μmol, 1.0 eq) in tetrahydrofuran (10 mL) was added HF Pyridine (350 mg, 3.53 mmol, 5 eq) at 0° C. The resulting reaction mixture was stirred for 16 h at room temperature. The progress of reaction mass was monitored by ELSD/TLC (SM was consumed). The resulting reaction mixture was quenched with cold aqueous sodium bicarbonate solution (20 mL) and extract with ethyl acetate (3×25 mL). The resulting organic layer was dried over sodium sulphate and concentrated under reduce pressure. The crude was purified by flash column chromatography (SiO₂: 0-20% methanol in dichloromethane) to get 2-(2,6-didodecylmorpholin-4-yl)ethan-1-ol (0.11 g) as colourless liquid. The compound (0.11 g) was obtained after column chromatography was dissolved in dichloromethane (10 mL) and was filtered through hydrophobic PTFE syringe filter (25 mm×0.45 μm) using glass syringe to get 2-(2,6-didodecylmorpholin-4-yl)ethan-1-ol (0.1 g, 30.26%, Yield) as colourless liquid. ¹H NMR (400 MHz, CDCl₃-d3): δ 3.61 (t, J=5.2 Hz, 2H), 3.48-3.43 (m, 2H), 2.74 (d, J=10.4 Hz, 2H), 2.51 (t, J=5.2 Hz, 2H), 1.83 (t, J=10.8 Hz, 2H), 1.50-1.25 (m, 44H), 0.87 (t, J=6.8 Hz, 6H). ELSD analysis: Purity 99.86%, Calculated C₃₀H₆₂NO₂=468.47, Observed=468.45 (m/z, M+H⁺)

### Example 205. Synthesis of Compound 91

Step 1: Synthesis of 1-((2-((tert-butyldimethylsilyl)oxy)ethyl)amino)tetradecan-2-ol

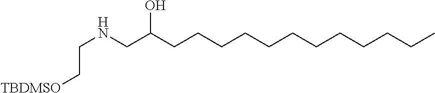 [SMILES: CCCCCCCCCCCCC(O)CNCCO[Si](C)(C)C(C)(C)C]

A mixture of (2-aminoethoxy)(tert-butyl)dimethylsilane (2 g, 11.4 mmol, 1.0 eq) and 2-dodecyloxirane (2.3 g, 10.8 mmol, 0.95 eq) in isopropanol (25 mL) was heated under nitrogen atmosphere to 55° C. for 16 h. The progress of reaction was monitored by ELSD/TLC (SM was consumed). The reaction mixture was concentrated, and the crude was purified by flash column chromatography (SiO₂: 0-20% methanol in dichloromethane) to obtain 1-({2-[(tert-butyldimethylsilyl)oxy]ethyl}amino)tetradecan-2-ol (2.5 g, 56.53%, Yield) as yellow liquid. ELSD analysis: Purity 99.67%, Calculated C₂₂H₅₀NO₂Si, [M+H]=388.35, Observed=388.30 (m/z, M+H⁺).

Step 2: Synthesis of Z)-1-((2-((tert-butyldimethylsilyl)oxy)ethyl)(2-hydroxytetradecyl)amino)octadec-9-en-2-ol

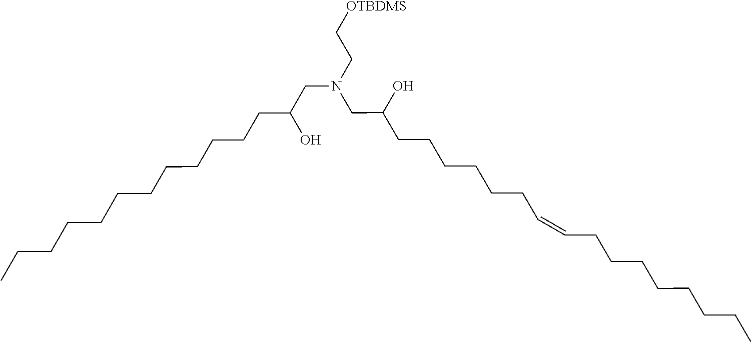 [SMILES: CCCCCCCC/C=C\CCCCCCC(O)CN(CCO[Si](C)(C)C(C)(C)C)CC(O)CCCCCCCCCCCC]

A mixture of 1-({2-[(tert-butyldimethylsilyl)oxy]ethyl}amino)tetradecan-2-ol (0.5 g, 1.29 mmol, 1.0 eq) and 2-[(7Z)-hexadec-7-en-1-yl]oxirane (378 mg, 1.42 mmol, 1.1 eq) in isopropanol (10 mL) was heated under nitrogen atmosphere to 95° C. for 20 h. The progress of reaction was monitored by ELSD/TLC (SM was consumed). The reaction mixture was concentrated, and the crude was purified by flash column chromatography (SiO₂: 0-20% methanol in dichloromethane) to obtain (9Z)-1-({2-[(tert-butyldimethylsilyl)oxy]ethyl}(2-hydroxytetradecyl)amino)octadec-9-en-2-ol (550 mg, 65.19%, Yield) as yellow liquid. ELSD) analysis: Purity 95.37%, Calculated C₄₀H₈₄NO₃Si, [M+H]=654.61, Observed=654.50 (m/z, M+H⁺).

Step 3: Synthesis of (Z)-4-(2-((tert-butyldimethylsilyl)oxy)ethyl)-2-dodecyl-6-(hexadec-7-en-1-yl)morpholine

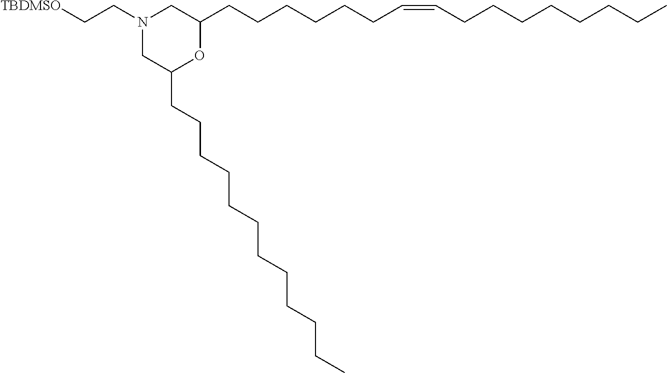 [SMILES: CCCCCCCC/C=C\CCCCCCC1CN(CCO[Si](C)(C)C(C)(C)C)CC(CCCCCCCCCCCC)O1]

To a stirred solution of (9Z)-1-({2-[(tert-butyldimethylsilyl)oxy]ethyl}(2-hydroxytetradecyl)amino)octadec-9-en-2-ol (4.2 g, 6.42 mmol, 1.0 eq) in toluene (25 mL), triphenylphosphane (2.02 g, 7.7 mmol, 1.2 eq.) was added portion wise. After 10 minutes N-{[(propan-2-yloxy)carbonyl]imino}(propan-2-yloxy)formamide (1.56 g, 7.7 mmol, 1.2 eq) was added dropwise at the same reaction mixture. The reaction mixture was stirred at 40° C. for 16 hrs. The reaction was monitored by ELSD & TLC. After completion of reaction, reaction was quenched with water (50 ml) and extracted with ethyl acetate (250 ml). Organic layer was dried over anhydrous sodium sulphate and concentrated under reduced vacuum. The crude was purified by column chromatography (0-10% Ethyl Acetate in Hexane) to give 4-{2-[(tert-butyldimethylsilyl)oxy]ethyl}-2-dodecyl-6-[(7Z)-hexadec-7-en-1-yl]morpholine (1.1 g, 26.93%, Yield) as colourless liquid. ELSD analysis: Purity 97.68%, Calculated C₄₀H₈₂NO₂Si, [M+H]=636.30, Observed=636.45 (m/z, M+H⁺).

Step 4: Synthesis of (Z)-2-(2-dodecyl-6-(hexadec-7-en-1-yl)morpholino)ethan-1-ol

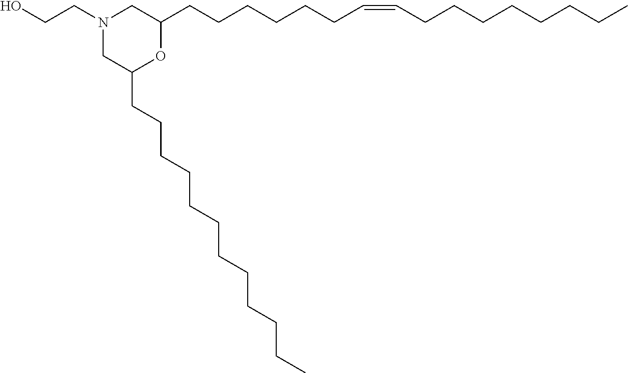 [SMILES: CCCCCCCC/C=C\CCCCCCC1CN(CCO)CC(CCCCCCCCCCCC)O1]

To a stirred solution of 4-{2-[(vc-butyldimethylsilyl)oxy]ethyl}-2-dodecyl-6-[(7Z)-hexadec-7-en-1-yl]morpholine (0.5 g, 786 μmol, 1.0 eq) in tetrahydrofuran (10 mL) was added hydrogen fluoride-pyridine (1/1) (389 mg, 3.93 mmol, 5 eq.) at 0° C. The resulting reaction mixture was stirred for 16 h at room temperature. The progress of reaction mass was monitored by ELSD/TLC (SM was consumed). The resulting reaction mixture was quenched with cold aqueous sodium bicarbonate solution (20 mL) and extract with ethyl acetate (3×25 mL). The resulting organic layer was dried over sodium sulphate and concentrated under reduce pressure, and the crude was purified by flash column chromatography (SiO₂: 0-20% methanol in dichloromethane), to obtain 2-{2-dodecyl-6-[(7Z)-hexadec-7-en-1-yl]morpholin-4-yl}ethan-1-ol (190 mg) as colourless liquid. The compound (0.190 mg) was obtained after column chromatography was dissolved in dichloromethane (10 mL) and was filtered through hydrophobic PTFE syringe filter (25 mm×0.45 μm) using glass syringe to get 2-{2-dodecyl-6-[(7Z)-hexadec-7-en-1-yl]morpholin-4-yl}ethan-1-ol (180 mg, 43.88%, Yield) as colourless liquid. ¹H NMR (400 MHz, CDCl₃-d3): δ 5.38-5.30 (m, 2H), 3.61 (t, J=5.2 Hz, 2H), 3.48-3.43 (m, 2H), 2.74 (d, J=10.8 Hz, 2H), 2.51 (t, J=5.2 Hz, 2H), 2.01-1.98 (m, 4H), 1.83 (t, J=10.8 Hz, 2H), 1.50-1.25 (m, 44H), 0.87 (t, J=6.8 Hz, 6H). ELSD analysis: Purity 99.91%, Calculated C₃₄H₆₈NO₂=522.52, Observed=522.50 (m/z, M+H⁺).

### Example 206. Synthesis of Compound 92

Step 1: Synthesis of (9Z,9′Z)-1,1′-((2-((tert-butyldimethylsilyl)oxy)ethyl)azanediyl)bis(octadec-9-en-2-ol)

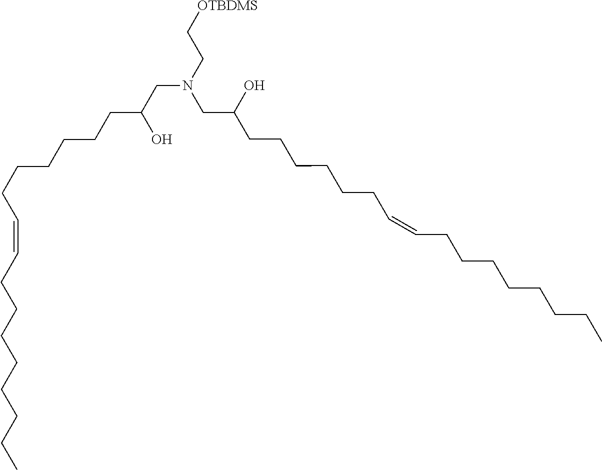 [SMILES: CCCCCCCC/C=C\CCCCCCC(O)CN(CCO[Si](C)(C)C(C)(C)C)CC(O)CCCCCC/C=C\CCCCCCCC]

A mixture of (2-aminoethoxy)(tert-butyl)dimethylsilane (4 g, 22.8 mmol, 1.0 eq) and 2-[(Z)-7-hexadecenyl]oxirane (13.4 g, 50.2 mmol, 2.2 eq) in isopropanol (0.2 L) was heated under nitrogen atmosphere to 95° C. for 16 h. The progress of reaction was monitored by ELSD/TLC (SM was consumed). The reaction mixture was concentrated, and the crude was purified by flash column chromatography (SiO₂: 0-20% methanol in dichloromethane) to obtain the desired (9Z)-1-({2-[(tert-butyldimethylsilyl)oxy]ethyl}[(9Z)-2-hydroxyoctadec-9-en-1-yl]amino)octadec-9-en-2-ol (10.2 g, 63.11%, Yield) as yellow liquid. ELSD analysis: Purity 99.91%, Calculated C₄₄H₉₀NO₃Si, [M+H]=708.66, Observed=708.50 (m/z, M+H⁺).

Step 2: Synthesis of 4-(2-((tert-butyldimethylsilyl)oxy)ethyl)-2,6-di((Z)-hexadec-7-en-1-yl)morpholine

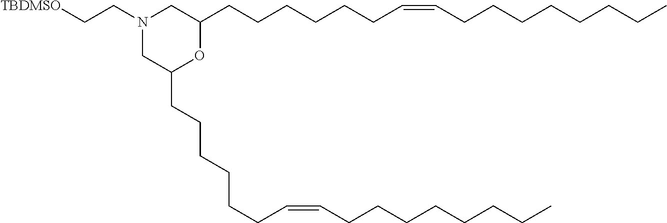 [SMILES: CCCCCCCC/C=C\CCCCCCC1CN(CCO[Si](C)(C)C(C)(C)C)CC(CCCCCC/C=C\CCCCCCCC)O1]

To a stirred solution of (9Z)-1-({2-[(tert-butyldimethylsilyl)oxy]ethyl}[(9Z)-2-hydroxyoctadec-9-en-1-yl]amino)octadec-9-en-2-ol (5 g, 7.06 mmol, 1.0 eq) in toluene (50 mL) was added triphenylphosphane (2.22 g, 8.47 mmol, 1.2 eq) portion wise. After 10 minutes N-{[(propan-2-yloxy)carbonyl]imino}(propan-2-yloxy)formamide (1.48 g, 8.47 mmol, 1.2 eq) was added dropwise at same reaction mixture. The reaction mixture was stirred at 40° C. for 16 hrs. The reaction was monitored by ELSD & TLC. After completion of reaction quenched with water (50 ml) and extracted with ethyl acetate (250 ml). Organic layer was dried over anhydrous sodium sulphate and concentrated under reduced vacuum. The crude was purified by column chromatography (0-10% Ethyl Acetate in Hexane) to give 4-{2-[(tert-butyldimethylsilyl)oxy]ethyl}-2,6-bis[(7Z)-hexadec-7-en-1-yl]morpholine (1.5 g, 30.78%, Yield) as colourless liquid.

ELSD analysis: Purity 89.61%, Calculated C₄₄H₈₈NO₂Si, [M+H]=690.65, Observed=690.50 (m/z, M+H⁺).
Step 3: Synthesis of 2-(2,6-di((Z)-hexadec-7-en-1-yl)morpholino)ethan-1-ol

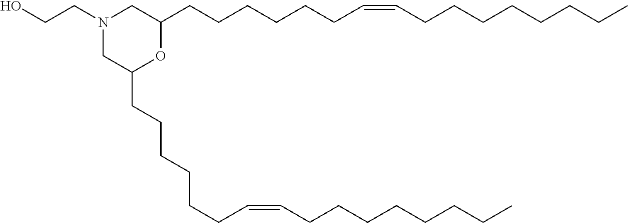 [SMILES: CCCCCCCC/C=C\CCCCCCC1CN(CCO)CC(CCCCCC/C=C\CCCCCCCC)O1]

To a stirred solution of 4-{2-[(tert-butyldimethylsilyl)oxy]ethyl}-2,6-bis[(7Z)-hexadec-7-en-1-yl]morpholine (315 mg, 456 μmol, 1.0 eq) in tetrahydrofuran (5 mL) was added hydrogen fluoride-pyridine (1/1) (226 mg, 2.28 mmol, 5 eq) at 0° C. The resulting reaction mixture was stirred for 16 h at room temperature. The progress of reaction mass was monitored by ELSD/TLC (SM was consumed). The resulting reaction mixture was quenched with cold aqueous sodium bicarbonate solution (20 mL) and extract with ethyl acetate (3×25 mL). The resulting organic layer was dried over sodium sulphate and concentrated under reduce pressure, and the crude was purified by flash column chromatography (SiO₂: 0-20% methanol in dichloromethane), to obtain 2-{2,6-bis[(7Z)-hexadec-7-en-1-yl]morpholin-4-yl}ethan-1-ol (120 mg) as colourless liquid. The compound (0.120 g) was obtained after column chromatography was dissolved in dichloromethane (10 mL) and was filtered through hydrophobic PTFE syringe filter (25 mm×0.45 μm) using glass syringe to get 2-{2,6-bis[(7Z)-hexadec-7-en-1-yl]morpholin-4-yl}ethan-1-ol (110 mg, 41.85%, Yield) as colourless liquid. ¹H NMR (400 MHz, CDCl₃-d3): δ 5.37-5.30 (m, 4H), 3.61 (t, J=5.2 Hz, 2H), 3.48-3.43 (m, 2H), 2.74 (d, J=10.4 Hz, 2H), 2.51 (t, J=5.2 Hz, 2H), 2.03-1.98 (m, 8H), 1.83 (t, J=10.8 Hz, 2H), 1.50-1.38 (m, 4H), 1.30-1.26 (m, 41H), 0.87 (m, 6H). ELSD analysis: Purity 99.77%, Calculated C₃₈H₇₄NO₂=576.56, Observed=576.50 (m/z, M+H⁺).

Example 207. Synthesis of Compound 93: -(2,6-di((Z)-hexadec-7-en-1-yl)morpholino)ethyl 4-(dimethylamino)butanoate

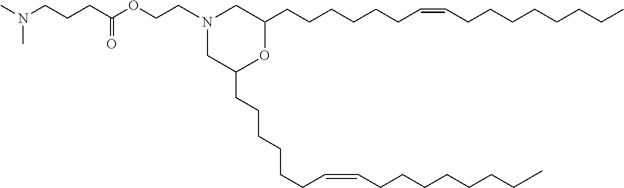 [SMILES: CCCCCCCC/C=C\CCCCCCC1CN(CCOC(=O)CCCN(C)C)CC(CCCCCC/C=C\CCCCCCCC)O1]

To a stirred solution of 4-(dimethylamino)butyric acid (708 mg, 5.4 mmol, 4 eq.) in dichloromethane (10 ml), was added {3-[cyano(ethyl)amino]propyl}dimethylazanium chloride (1.04 g, 5.4 mmol, 4 eq) and 4-(dimethylamino)pyridin-1-ium (247 mg, 2.03 mmol, 1.5 eq). The reaction mixture was stirred for 40 min, then added 2-{2,6-bis[(Z)-7-hexadecenyl]-4-morpholinyl}ethanol (0.7 g, 1.22 mmol, 0.9 eq). The reaction mixture was stirred at room temperature for 16 h. The progress of reaction was monitored by TLC/ELSD. The reaction mixture was quenched with brine solution and extracted with DCM (10 mL×2). The combined organic layer was dried over sodium sulphate, filtered and concentrated to get the crude mixture. The crude was diluted with pentane and washed with acetonitrile (10 mL×3). Pentane layer was concentrated under vacuum (temp. below 30° C.) to get 2-{2,6-bis[(Z)-7-hexadecenyl]-4-morpholinyl}ethyl 4-(dimethylamino)butyrate (350 mg) as colourless liquid. The compound (110 mg) was dissolved in dichloromethane (10 mL) and was filtered through hydrophobic PTFE syringe filter (25 mm×0.45 μm) using glass syringe to get desired 2-{2,6-bis[(Z)-7-hexadecenyl]-4-morpholinyl}ethyl 4-(dimethylamino)butyrate (350 mg, 37%, Yield) as a colourless oil. ¹H-NMR (400 MHz, CDCl₃-d₃): δ 5.37-5.29 (m, 4H), 4.19 (t, J=6.0 Hz, 2H), 3.48-3.42 (m, 2H), 2.75 (d, J=10.4 Hz, 2H), 2.59 (t, J=6.0 Hz, 2H), 2.37-2.31 (dt, J=11.6, 4.0 Hz, 4H), 2.22 (s, 6H), 2.05-1.97 (m, 8H), 1.88-1.75 (m, 4H), 1.50-1.42 (m, 4H), 1.29-1.26 (br, 40H), 0.88-0.85 (m, 6H). ELSD analysis: Purity 99.98%, Calculated C₄₄H₈₅N₂O₃, [M+H]=689.65, Observed=689.55 (m/z, M+H⁺).

### Example 208. Synthesis of Compound 94

Step 1: Synthesis of (Z)-1-((2-((tert-butyldimethylsilyl)oxy)ethyl)amino)octadec-9-en-2-ol

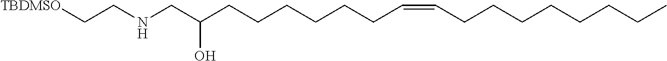 [SMILES: CCCCCCCC/C=C\CCCCCCC(O)CNCCO[Si](C)(C)C(C)(C)C]

A mixture of (2-aminoethoxy)(tert-butyl)dimethylsilane (3 g, 17.1 mmol, 1.0 eq) and 2-[(Z)-7-hexadecenyl]oxirane (4.1 g, 15.4 mmol, 0.9 eq) in isopropanol (50 mL) was stirred under nitrogen atmosphere to r.t. for 16 h. The progress of reaction was monitored by ELSD/TLC (SM was consumed). The reaction mixture was concentrated under vacuum and the crude was purified by flash column chromatography (SiO₂: 0-20% methanol in dichloromethane) to obtain (Z)-2-(tert-butyl)-2-methyl-3-oxa-6-aza-2-sila-15-tetracosen-8-ol (2.1 g, 27.78%, Yield) as white semisolid. ELSD analysis: Purity 98.17%, Calculated C₂₆H₅₆NO₂Si, [M+H⁺]=442.40, Observed=442.45 (m/z, M+H⁺).

Step 2: Synthesis of heptadecan-9-yl (Z)-8-((2-((tert-butyldimethylsilyl)oxy)ethyl)(2-hydroxyoctadec-9-en-1-yl)amino)-7-hydroxyoctanoate

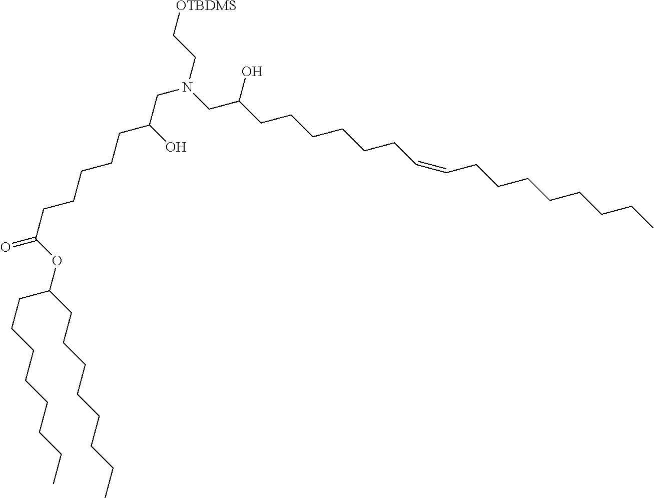 [SMILES: CCCCCCCC/C=C\CCCCCCC(O)CN(CCO[Si](C)(C)C(C)(C)C)CC(O)CCCCCC(=O)OC(CCCCCCCC)CCCCCCCC]

To the stirred solution of (Z)-2-(tert-butyl)-2-methyl-3-oxa-6-aza-2-sila-15-tetracosen-8-ol (550 mg, 1.24 mmol, 1.0 eq) in isopropanol (0.1 L) was added 1-octylnonyl 6-(2-oxiranyl)hexanoate (444 mg, 1.12 mmol, 0.9 eq) at room temperature. The reaction was allowed to stir at 90° C. for 16 h. The progress of reaction was monitored by ELSD/TLC (SM was consumed). The reaction mixture was concentrated under vacuum to get crude compound. The crude compound was purified by column chromatography using silica gel (SiO₂: 0-20% methanol in dichloromethane) to afford 1-octylnonyl 8-{[(Z)-2-hydroxy-9-octadecenyl]{2-[(tert-butyl)bis(methyl)siloxy]ethyl}amino}-7-hydroxyoctanoate (0.5 g, 47.9%, Yield) as yellow viscous liquid. ELSD analysis: Purity 97.69%, Calculated C₅₁H₁₀₄NO₅Si, [M+H⁺]=838.76, Observed=838.75 (m/z, M+H⁺).

Step 3: Synthesis of heptadecan-9-yl (Z)-6-(4-(2-((tert-butyldimethylsilyl)oxy)ethyl)-6-(hexadec-7-en-1-yl)morpholin-2-yl)hexanoate

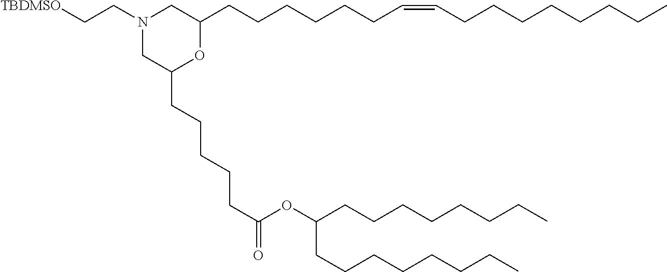 [SMILES: CCCCCCCC/C=C\CCCCCCC1CN(CCO[Si](C)(C)C(C)(C)C)CC(CCCCCC(=O)OC(CCCCCCCC)CCCCCCCC)O1]

To a stirred solution of 1-octylnonyl 8-{[(Z)-2-hydroxy-9-octadecenyl]{2-[(tert-butyl)bis(methyl)siloxy]ethyl}amino}-7-hydroxyoctanoate (550 mg, 656 μmol, 1.0 eq) in Toluene (40 mL) was added triphenylphosphine (206 mg, 787 μmol, 1.2 eq) portion wise. After 10 minutes N-{[(propan-2-yloxy)carbonyl]imino}(propan-2-yloxy)formamide (159 mg, 787 μmol, 1.2 eq) was added dropwise to the reaction mixture. The reaction mixture was stirred at 40° C. for 16 hrs. The reaction was monitored by ELSD & TLC. After completion of reaction quenched with water (50 ml) and extracted with ethyl acetate (250 ml). Organic layer was dried over anhydrous sodium sulphate and concentrated under reduced vacuum. The crude was purified by column chromatography (0-10% Ethyl Acetate in Hexane) to offered 1-octylnonyl 6-{6-[(Z)-7-hexadecenyl]-4-{2-[(tert-butyl)bis(methyl)siloxy]ethyl}-2-morpholinyl}hexanoate (250 mg, 46.45%, Yield) as colourless liquid. ELSD analysis: Purity 99.10%, Calculated C₅₁H₁₀₂NO₄Si, [M+H⁺]=820.75, Observed=820.75 (m/z, M+H⁺).

Step 4: Synthesis of heptadecan-9-yl (Z)-6-(6-(hexadec-7-en-1-yl)-4-(2-hydroxyethyl)morpholin-2-yl)hexanoate

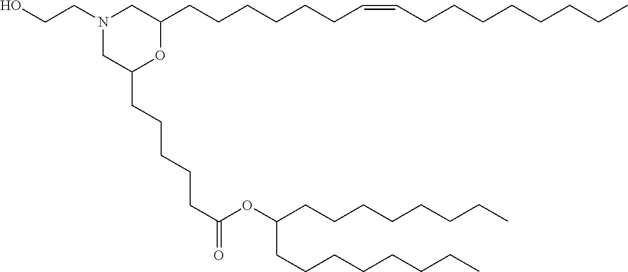 [SMILES: CCCCCCCC/C=C\CCCCCCC1CN(CCO)CC(CCCCCC(=O)OC(CCCCCCCC)CCCCCCCC)O1]

To a stirred solution of 1-octylnonyl 6-{6-[(Z)-7-hexadecenyl]-4-{2-[(tert-butyl)bis(methyl)siloxy]ethyl}-2-morpholinyl}hexanoate (130 mg, 158 μmol, 1.0 eq) in tetrahydrofuran (10 mL) was added hydrogen fluoride-pyridine (1/1)(71.4 μL, 792 μmol, 5 eq) at 0° C. The resulting reaction mixture was stirred for 16 h at room temperature. Progress of reaction mass was monitored by ELSD/TLC (SM was consumed). The resulting reaction mixture was quenched with cold aqueous sodium bicarbonate solution (20 mL) and extracted with ethyl acetate (3×25 mL). The resulting organic layer was dried over sodium sulphate and concentrated under reduce pressure. The crude was purified by flash column chromatography (SiO₂: 0-30% Ethyl acetate in Hexane), to obtain the desired produced 1-octylnonyl 6-{6-[(Z)-7-hexadecenyl]-4-(2-hydroxyethyl)-2-morpholinyl}hexanoate (97 mg, 86.69%, Yield) as yellow liquid. ELSD analysis: Purity 97.76%, Calculated C₄₅H₈₈NO₄=706.66, Observed=706.65 (m/z, M+H⁺).

Step 5: Synthesis of heptadecan-9-yl (Z)-6-(4-(2-((4-(dimethylamino)butanoyl)oxy)ethyl)-6-(hexadec-7-en-1-yl)morpholin-2-yl)hexanoate

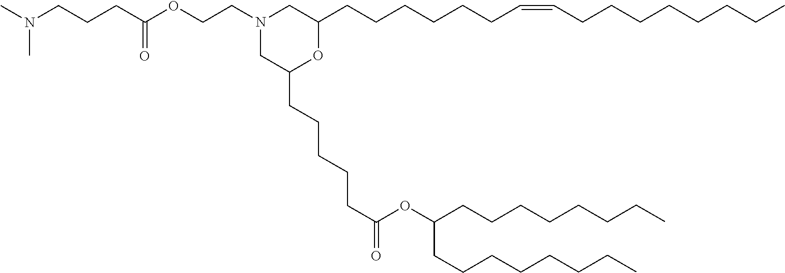 [SMILES: CCCCCCCC/C=C\CCCCCCC1CN(CCOC(=O)CCCN(C)C)CC(CCCCCC(=O)OC(CCCCCCCC)CCCCCCCC)O1]

To a stirred solution of 4-(dimethylamino)butyric acid (90.1 mg, 687 μmol, 5 eq) in dichloromethane (25 mL, 390 mmol) was added {3-[cyano(ethyl)amino]propyl}dimethylazanium chloride (132 mg, 5 eq., 687 μmol) and N,N-dimethyl-4-pyridylamine (33.6 mg. 2 eq., 275 μmol). The reaction mixture was stirred for 40 min, then added 1-octylnonyl 6-{6-[(Z)-7-hexadecenyl]-4-(2-hydroxyethyl)-2-morpholinyl}hexanoate (97 mg, 137 μmol). The reaction mixture was stirred at r.t. for 16 h. The progress of reaction was monitored by TLC/ELSD. The reaction mixture was quenched with brine solution and extracted with DCM (10 mL×2). The combined organic layer was dried over sodium sulphate, filtered and concentrated to get the crude mixture. The crude was diluted with pentane and washed with acetonitrile (10 mL×3). Pentane layer was distilled (temp. below 30° C.) under vacuum to get 1-octylnonyl 6-{6-[(Z)-7-hexadecenyl]-4-{2-[4-(dimethylamino)butyroxy]ethyl}-2-morpholinyl}hexanoate (90 mg) as colourless liquid. The compound (90 mg) was dissolved in dichloromethane (10 mL) and was filtered through hydrophobic PTFE syringe filter (25 mm×0.45 μm) using glass syringe to get desired 1-octylnonyl 6-{6-[(Z)-7-hexadecenyl]-4-{2-[4-(dimethylamino)butyroxy]ethyl}-2-morpholinyl}hexanoate (85 mg, 75.53%, Yield). ¹H-NMR (400 MHz, CDCl₃-d₃): δ 5.38-5.30 (m, 2H), 4.88-4.82 (m, 1H), 4.23-4.01 (m, 2H), 3.70 (s, 2H), 3.45-3.42 (m, 2H), 2.56-2.46 (m, 6H), 2.41-2.37 (m, 6H), 2.77 (t, J=7.6 Hz, 2H), 2.23-2.19 (m, 4H), 2.03-2.00 (m, 3H), 1.74-1.62 (m, 6H), 1.53-1.47 (m, 4H), 1.43-1.22 (m, 50H), 0.89-0.85 (m, 9H). ELSD analysis: Purity 98.68%, Calculated C₅₁H₉₉N₂O₅, [M+H⁺]=819.75, Observed=819.70 (m/z, M+H⁺).

Example 209. Synthesis of Compound 95: 2-(2,6-di((Z)-hexadec-7-en-1-yl)morpholino)ethyl 3-(dimethylamino)propanoate

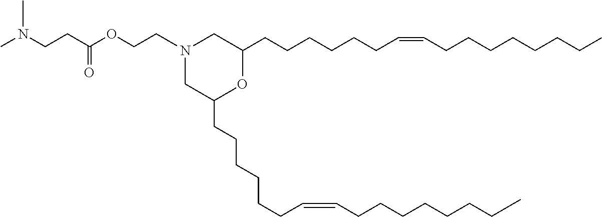 [SMILES: CCCCCCCC/C=C\CCCCCCC1CN(CCOC(=O)CCN(C)C)CC(CCCCCC/C=C\CCCCCCCC)O1]

To a stirred solution of 3-(dimethylamino) propionic acid (110 mg, 937 μmol, 4 eq) in dichloromethane (10 ml), was added {3-[cyano(ethyl)amino]propyl}dimethylazanium chloride (180 mg, 937 μmol, 4 eq) and 4-(dimethylamino)pyridin-1-ium (115 mg, 937 μmol, 4.0 eq) to it. The reaction mixture was stirred for 40 min, then 2-{2,6-bis[(Z)-7-hexadecenyl]-4-morpholinyl}ethanol (135 mg, 235 μmol, 1.0 eq) was added. The reaction mixture was stirred at r.t. for 16 h. The progress of reaction was monitored by TLC/ELSD. The reaction mixture was quenched with brine solution and extracted with DCM (10 mL×2). The combined organic layer was dried over sodium sulphate, filtered and concentrated to get the crude mixture. The crude was diluted with pentane and washed with acetonitrile (10 mL×3). Pentane layer was evaporated and distilled (temp. below 30° C.) to get 2-{2,6-bis[(Z)-7-hexadecenyl]-4-morpholinyl}ethyl 3-(dimethylamino)propionate (115 mg) as colourless liquid. The compound (115 mg) was dissolved in dichloromethane (10 mL) and was filtered through hydrophobic PTFE syringe filter (25 mm×0.45 μm) using glass syringe to get desired 2-{2,6-bis[(Z)-7-hexadecenyl]-4-morpholinyl}ethyl 3-(dimethylamino)propionate (90 mg, 56.88%, Yield). ¹H-NMR (400 MHz, CDCl₃-d₃): δ 5.38-5.29 (m, 4H), 4.21 (t, J=6.0 Hz, 2H), 3.49-3.43 (m, 2H), 2.75 (d, J=10.4 Hz, 2H), 2.62-2.58 (m, 4H), 2.49 (t, J=6.8 Hz, 2H), 2.23 (s, 6H), 2.05-1.97 (m, 8H), 1.80 (t, J=10.4 Hz, 2H), 1.50-1.40 (m, 4H), 1.29-1.26 (br, 40H), 0.88-0.85 (m, 6H). ELSD analysis: Purity 96.78%, Calculated C₄₃H₈₃N₂O₃, [M+H]=675.63, Observed=675.55 (m/z, M+H⁺).

### Example 210. Synthesis of Compound 96

Step 1: Synthesis of 3-((tert-butyldimethylsilyl)oxy)propan-1-amine

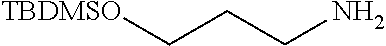 [SMILES: CC(C)(C)[Si](C)(C)OCCCN]

To a stirred solution of 3-amino-1-propanol (10 g, 133 mmol, 1.0 eq) in dichloromethane (500 mL) was added 1H-imidazole (36.3 g, 533 mmol, 4 eq) at room temperature. The resulting reaction mixture was cooled to 0° C. and added tert-butyl(chloro)dimethylsilane (40.1 g, 266 mmol, 2 eq.) portion wise at same temperature. The reaction mixture was stirred for 16 h at room temperature. Progress of reaction was monitored by ELSD/TLC. Water (500 mL) was added to the reaction mixture and extracted with DCM (3×500 mL). All organic layers were combined and washed with brine solution (2×500 mL). The resulting organic layer was dried over anhydrous sodium sulphate and concentrated under reduce pressure to obtained crude product which was purified over silica (10% methanol in dichloromethane) to obtained pure product (21 g, 83.29%, Yield) as a colour less liquid. ELSD analysis: Purity 96.70%, Calculated C₉H₂₄NOSi, [M+H⁺]=190.15, Observed=190.35 (m/z, M+H⁺).

Step 2: Synthesis of (9Z,9′Z)-1,1′-((3-((tert-butyldimethylsilyl)oxy)propyl)azanediyl)bis(octadec-9-en-2-ol)

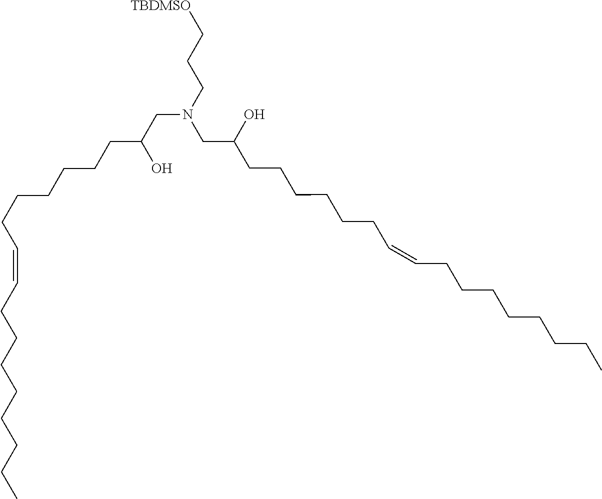 [SMILES: CCCCCCCC/C=C\CCCCCCC(O)CN(CCCO[Si](C)(C)C(C)(C)C)CC(O)CCCCCC/C=C\CCCCCCCC]

A mixture of (2-aminoethoxy)(tert-butyl)dimethylsilane (1.0 g, 5.28 mmol, 1.0 eq) and 2-[(Z)-7-hexadecenyl]oxirane (3.52 g, 13.2 mmol, 2.5 eq) in isopropanol (30 mL) was heated under nitrogen atmosphere to 95° C. for 16 h. The progress of reaction was monitored by ELSD/TLC (SM was consumed). The reaction mixture was concentrated under vacuum and the crude was purified by flash column chromatography (SiO₂: 0-20% methanol in dichloromethane) to obtained the desired (9Z,9′Z)-1,1′-((3-((tert-butyldimethylsilyl)oxy)propyl)azanediyl)bis(octadec-9-en-2-ol) (1.3 g, 34.04%, Yield) as colorless liquid. ELSD analysis: Purity 93.59%, Calculated C₄₅H₉₂NO₃Si, [M+H⁺]=722.68, Observed=722.70 (m/z, M+H⁺).

Step 3: Synthesis of 4-(3-((tert-butyldimethylsilyl)oxy)propyl)-2,6-di((Z)-hexadec-7-en-1-yl)morpholine

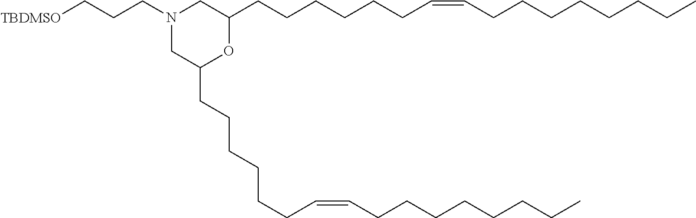 [SMILES: CCCCCCCC/C=C\CCCCCCC1CN(CCCO[Si](C)(C)C(C)(C)C)CC(CCCCCC/C=C\CCCCCCCC)O1]

To a stirred solution of (Z)-1-{[(Z)-2-hydroxy-9-octadecenyl]{3-[(tert-butyl)bis(methyl)siloxy]propyl}amino}-9-octadecen-2-ol (2.8 g, 3.88 mmol, 1.0 eq) in Toluene (50 mL) was added triphenylphosphane (1.22 g, 4.65 mmol, 1.2 eq) portion wise. After 10 minutes N-{[(propan-2-yloxy)carbonyl]imino}(propan-2-yloxy)formamide (705 mg, 3.49 mmol, 1.2 eq.) was added dropwise to the reaction mixture. The reaction mixture was stirred at 40° C. for 16 hrs. The reaction was monitored by ELSD & TLC. After completion of reaction quenched with water (50 ml) & extracted with ethyl acetate (250 ml). Organic layer was dried over anhydrous sodium sulphate and concentrated under reduced vacuum. The crude was purified by column chromatography (0-10% Ethyl Acetate in Hexane) to offered 4-(3-((tert-butyldimethylsilyl)oxy)propyl)-2,6-di((Z)-hexadec-7-en-1-yl)morpholine (1.2 g, 43.95%, Yield) as colourless liquid. ELSD analysis: Purity 93.44%, Calculated C₄₅H₉₀NO₂Si. [M+H⁺]=704.67, Observed=704.35 (m/z, M+H).

Step 4: Synthesis of 3-(2,6-di((Z)-hexadec-7-en-1-yl)morpholino)propan-1-ol

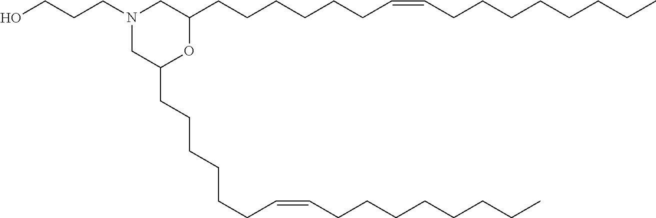 [SMILES: CCCCCCCC/C=C\CCCCCCC1CN(CCCO)CC(CCCCCC/C=C\CCCCCCCC)O1]

To a stirred solution of (3-{2,6-bis[(Z)-7-hexadecenyl]-4-morpholinyl}propoxy) (tert-butyl)bis(methyl)silane (330 mg, 469 μmol, 1.0 eq) in tetrahydrofuran (10 mL) was added, hydrogen fluoride-pyridine (1/1)(232 mg, 2.34 mmol, 5 eq) was added at 0° C. The resulting reaction mixture was stirred for 16 h at room temperature. Progress of reaction mass was monitored by ELSD/TLC (SM was consumed). The resulting reaction mixture was quenched with cold aqueous sodium bicarbonate solution (20 mL) and extracted with ethyl acetate (3×25 mL). The resulting organic layer was dried over sodium sulphate and concentrated under reduce pressure, and the crude was purified by flash column chromatography (SiO₂: 0-20% Ethyl Acetate in Hexane), to obtain the desired produced 3-{2,6-bis[(Z)-7-hexadecenyl]-4-morpholinyl}-1-propanol (130 mg, 47.02%, Yield) as colourless liquid. ELSD analysis: Purity 96.50%, Calculated C₃₉H₇₆NO₂=590.58, Observed=590.55 (m/z, M+H⁺).

Step 5: Synthesis of 3-(2,6-di((Z)-hexadec-7-en-1-yl)morpholino)propyl 3-(dimethylamino)propanoate

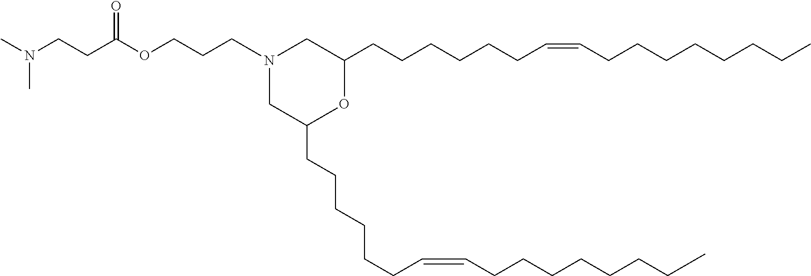 [SMILES: CCCCCCCC/C=C\CCCCCCC1CN(CCCOC(=O)CCN(C)C)CC(CCCCCC/C=C\CCCCCCCC)O1]

To a stirred solution of 3-(dimethylamino) propionic acid (127 mg, 1.08 mmol, 4 eq) in dichloromethane (10 ml) was added {3-[cyano(ethyl)amino]propyl}dimethylazanium chloride (208 mg, 1.08 mmol, 4 eq) and 4-(dimethylamino)pyridin-1-ium (133 mg, 1.08 mmol, 4 eq). The reaction mixture was stirred for 40 min at RT, then added 3-{2,6-bis[(Z)-7-hexadecenyl]-4-morpholinyl}-1-propanol (160 mg, 271 μmol 1.0 eq). The reaction mixture was stirred at r.t. for 16 h. The progress of reaction was monitored by TLC/ELSD. The reaction mixture was quenched with brine solution and extracted with DCM (10 mL×2). The combined organic layer was dried over sodium sulphate, filtered and concentrated to get the crude mixture. The crude was diluted with pentane and washed with acetonitrile (10 mL×3). Pentane layer was concentrated (temp. below 30° C.) to get 3-{2,6-bis[(Z)-7-hexadecenyl]-4-morpholinyl}propyl 3-(dimethylamino)propionate (115 mg) as pale yellow liquid. The compound (115 mg) was dissolved in dichloromethane (10 mL) and was filtered through hydrophobic PTFE syringe filter (25 mm×0.45 μm) using glass syringe to get 3-{2,6-bis[(Z)-7-hexadecenyl]-4-morpholinyl}propyl 3-(dimethylamino)propionate (109 mg, 58.83%, Yield) as pale yellow liquid. ¹H-NMR (400 MHz, CDCl₃-d₃): δ 5.39-5.29 (m, 4H), 4.14 (t, J=6.4 Hz, 2H), 3.49-3.45 (m, 2H), 2.75 (d, J=10.4 Hz, 2H), 2.68 (t, J=7.2 Hz, 2H), 2.52 (t, J=7.2 Hz, 2H), 2.40 (t, J=7.6 Hz, 2H), 2.29 (s, 6H), 2.05-1.97 (m, 8H), 1.85-1.80 (m, 4H), 1.72 (t, J=10.4 Hz, 2H), 1.52-1.26 (br, 44H), 0.89-0.85 (m, 6H). ELSD analysis: Purity 95.35%, Calculated C₄₄H₈₅N₂O₃, [M+H⁺]=689.63, Observed=689.60 (m/z, M+H⁺).

Example 211. Synthesis of Compound 97: heptadecan-9-yl (Z)-6-(4-(2-((3-(dimethylamino)propanoyl)oxy)ethyl)-6-(hexadec-7-en-1-yl)morpholin-2-yl)hexanoate

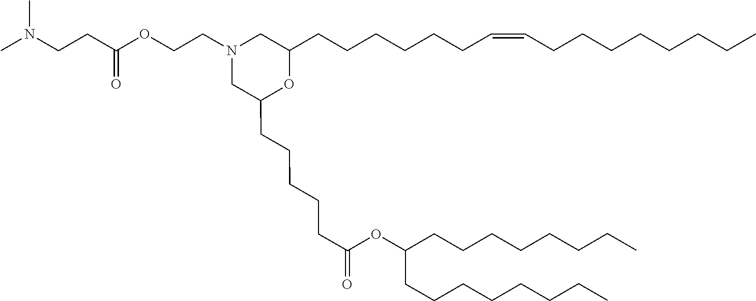 [SMILES: CCCCCCCC/C=C\CCCCCCC1CN(CCOC(=O)CCN(C)C)CC(CCCCCC(=O)OC(CCCCCCCC)CCCCCCCC)O1]

To a stirred solution of 3-(dimethylamino) propionic acid—hydrogen chloride (1/1) (221 mg. 1.15 mmol, 8 eq) in dichloromethane (15 ml), was added {3 [cyano(ethyl)amino]propyl}dimethylazanium chloride (138 mg, 718 μmol, 4 eq) and 4-(dimethylamino)pyridin-1-ium (88.5 mg. 718 μmol, 4 eq). The reaction mixture was stirred for 40 min, then added heptadecan-9-yl (Z)-6-(6-(hexadec-7-en-1-yl)-4-(2-hydroxyethyl)morpholin-2-yl)hexanoate (130 mg, 180 μmol, 1.0 eq). The reaction mixture was stirred at r.t. for 16 h. The progress of reaction was monitored by TLC/ELSD. The reaction mixture was quenched with brine solution and extracted with DCM (10 mL×2). The combined organic layer was dried over sodium sulphate, filtered and concentrated to get the crude mixture. The crude was diluted with pentane and washed with acetonitrile (10 mL×3). Pentane layer was distilled (temp. below 30° C.) under vacuum to get heptadecan-9-yl 6-(4-(2-((3-(dimethylamino)propanoyl)oxy)ethyl)-6-(6-(nonyloxy)-6-oxohexyl)morpholin-2-yl)hexanoate (130 mg) as colourless liquid. The compound (130 mg) was dissolved in dichloromethane (10 mL) and was filtered through hydrophobic PTFE syringe filter (25 mm×0.45 μm) using glass syringe to get heptadecan-9-yl (Z)-6-(4-(2-((3-(dimethylamino)propanoyl)oxy)ethyl)-6-(hexadec-7-en-1-yl)morpholin-2-yl)hexanoate (108 mg, 73%, Yield) as colourless liquid. ¹H-NMR (400 MHz, CDCl₃-d₃): δ 4.85 (qu, J=6.4 Hz, 1H), 4.21 (t, J=6.0 Hz, 2H), 4.04 (t, J=6.0 Hz, 2H), 3.45-3.42 (m, 2H), 2.74 (d, J=10.8 Hz, 2H), 2.64-2.57 (m, 4H), 2.50-2.47 (m, 2H), 2.30-2.23 (m, 9H), 1.79 (t, J=10.4 Hz, 2H), 1.67-1.60 (m, 8H), 1.50-1.42 (m, 7H), 1.37-1.25 (br, 44H), 0.88-0.85 (m, 9H). ELSD analysis: Purity 98.36%, Calculated C₄₉H₉₅N₂O₇, [M+H]=823.71, Observed=823.65 (m/z, M+H⁺).

### Example 212. Synthesis of Compound 98

Step 1: Synthesis of 7-(benzyloxy)heptan-1-ol

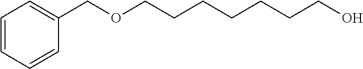 [SMILES: OCCCCCCCOCc1ccccc1]

To a stirred solution of 1,7-heptanediol (30 g, 227 mmol, 1.0 eq.) in dimethylformamide (0.3 L) was added sodium hydride (60% dispersion in mineral oil) (13.6 g, 340 mmol, 1.5 eq.) at 0° C. and stirred for 30 min at same temperature. Then (bromomethyl)benzene (27 ml, 227 mmol, 1.0 eq.) was added to it and the reaction mixture was stirred at r.t. for 16 h. The progress of reaction was monitored by TLC, after completion of the reaction the reaction mixture was quenched with ice-cooled water (0.2 L) and extracted with ethyl acetate (3×0.5 L). The resulting organic layer was dried over Na₂SO₄, and concentrated under reduce pressure. The crude was purified by flash column chromatography (SiO₂: 0-15% Ethyl acetate in Hexane) to give 7-(benzyloxy)-1-heptanol (21 g, Yield=41.62%) as a pale yellow liquid. ¹H NMR (400 MHz, DMSO-d6): δ 7.35-7.25 (m, 5H), 4.33 (s, 2H), 4.33-4.30 (t, J=5.2 Hz, 1H) 3.41-3.32 (m, 4H), 1.55-1.48 (m, 2H), 1.40-1.35 (m, 2H), 1.32-1.26 (m, 6H).

Step 2: Synthesis of 7-(benzyloxy)heptanal

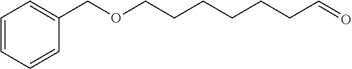 [SMILES: O=CCCCCCCOCc1ccccc1]

To a stirred solution of 7-(benzyloxy)-1-heptanol (28 g, 126 mmol, 1.0 eq.) in dichloromethane (500 mL), was add pyridinium chlorochromate (40.7 g, 189 mmol, 1.5 eq) at 0° C. The reaction mixture was stirred at r.t. for 2 h. The progress of reaction was monitored by TLC, the reaction mixture was diluted with pentane (1.0 L) and stirred for 30 min. After 30 min, mixture was filtered through celite bed and washed with pentane (3×500 ml). The organic layer was concentrated under reduced pressure. The crude was purified by flash column chromatography (SiO₂: 0-25% ethylacetate in hexane) to give 7-(benzyloxy)heptanal (20 g, Yield=72.08%) as a colorless liquid. ¹H NMR (400 MHz, CDCl₃-d3): δ 9.75 (m, 1H), 7.36-7.27 (m, 5H), 4.50 (s, 2H), 3.46 (t, J=6.4 Hz, 2H), 2.44-2.40 (m, 2H), 1.67-1.58 (m, 4H), 1.43-1.38 (m, 4H).

Step 3: Synthesis of (((8-methylnon-7-en-1-yl)oxy)methyl)benzene

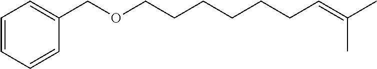 [SMILES: CC(C)=CCCCCCCOCc1ccccc1]

To a stirred solution of triphenyl(propan-2-yl)phosphanium bromide (36.6 g, 94.9 mmol, 1.1 eq) in tetrahydrofuran (600 mL) was added lithium 1-butanide (13.8 g, 216 mmol, 2.5 eq) dropwise at −78° C. The reaction mixture was stirred at room temperature for 1 hr, then the mixture was cooled again to −78° C. and added 7-(benzyloxy)heptanal (19 g, 86.2 mmol, 1.0 eq) (dissolved in 100 mL THF) was added dropwise. The reaction mixture was stirred at room temperature for 16 h. The progress of reaction was monitored by TLC. The reaction mixture was quenched with aq. ammonium chloride solution and extracted with ethyl acetate (3×500 mL). The organic layer was collected and dried over sodium sulphate, filtered and concentrated under vacuum to give the crude. The crude product was purified by flash column chromatography (SiO₂: 0-10% Ethyl acetate in Hexane) to give 9-(benzyloxy)-2-methyl-2-nonene (15.2 g, Yield=71.53%) as a colorless liquid. ¹H NMR (400 MHz, CDCl₃-d3): δ 7.36-7.27 (m, 5H), 5.13-5.09 (m, 1H), 4.50 (s, 2H), 3.46 (t, J=6.0 Hz, 2H), 1.96-1.92 (m, 2H), 1.70 (b, 3H), 1.68-1.58 (m, 6H), 1.38-1.27 (m, 5H).

Step 4: Synthesis of 8-methylnonan-1-ol

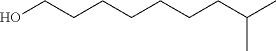

To a stirred solution of 9-(benzyloxy)-2-methyl-2-nonene (12.5 g, 50.7 mmol, 1.0 eq) in tetrahydrofuran (60 mL) and methanol (60 mL) was degassed with nitrogen for 15 min, then added Pd/C, 10%, 50% wet (13.5 g, w/w). The reaction mixture was stirred at r.t. for 48 h under Hydrogen atmosphere. TLC showed consumption of starting material and formation of a new spot. The reaction mixture was filtered through celite bed and washed with mixture of MeOH:THF (3×500 ml). The filtrate was collected and concentrate to get the crude. The crude was purified through flash column chromatography (SiO₂: 0-20% Ethyl acetate in Hexane) to get 8-methyl-1-nonanol (6.5 g, Yield=80.91%) as pale yellow liquid. ¹H NMR (400 MHz, DMSO-d6): δ 4.31 (t, J=5.2 Hz, 1H), 3.38-3.34 (m, 2H), 1.52-1.46 (m, 1H), 1.39-1.37 (bs, 2H), 1.24 (b, 8H), 1.13 (b, 2H), 0.84 (d, J=6.8 Hz, 6H).

Step 5: Synthesis of 8-methylnonanal

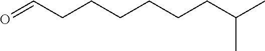

To a stirred solution of 8-methyl-1-nonanol (6.5 g, 41.1 mmol, 1.0 eq) in dichloromethane (150 mL) was added pyridinium chlorochromate (17.7 g, 82.1 mmol, 2 eq) at 0° C. and the reaction mixture was stirred at r.t. for 2 h. The progress of reaction was monitored by TLC (SM was consumed). The reaction mixture was diluted with pentane (500 ml) and stirred for 30 min. After 30 min, mixture was filtered through celite bed and wash with pentane (3×200 ml). The organic layer was collected and dried over sodium sulphate, filtered and concentrated under reduce pressure, to get the crude product 8-methylnonanal (6 g, Yield=93.5%) as a colorless liquid which was used for next step without further purification.

Step 6: Synthesis of 2-methylheptadecan-9-ol

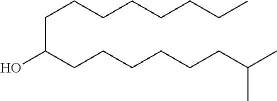 [SMILES: CCCCCCCCC(O)CCCCCCC(C)C]

To a stirred solution of 8-methylnonanal (6 g. 38.4 mmol, 1.0 eq) in tetrahydrofuran (120 mL) was added magnesium 1-octanide bromide (12.5 g, 57.6 mmol, 1.5 eq) (1M soln in diethyl ether) dropwise at −78° C. The reaction mixture was stirred at room temperature for 1 hr, then reaction mixture heated up to 55° C. for 1 hr. After completion of reaction (monitored by TLC), the reaction mixture was quenched with aq. ammonium chloride solution and extracted with ethyl acetate (100 mL×3) times. The organic layer was collected and dried over sodium sulphate, filtered and concentrated under vacuum to get the crude mixture. The crude product was purified through combi-flash chromatography (SiO₂: 0-15% Ethyl acetate in Hexane), to give 2-methyl-9-heptadecanol (6.5 g, Yield=62.58%) as a colorless liquid. ¹H-NMR (400 MHz, CDCl₃-d3): δ 3.58 (b, 1H), 1.54-1.38 (m, 8H), 1.28 (b, 18H), 1.16-1.15 (m, 2H), 0.89-0.85 (m, 9H).

Step 7: Synthesis of 2-methylheptadecan-9-yl oct-7-enoate

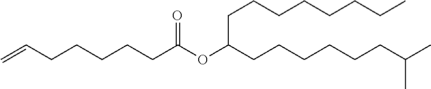 [SMILES: C=CCCCCCC(=O)OC(CCCCCCCC)CCCCCCC(C)C]

A stirred solution of oct-7-enoic acid (4.36 g, 30.7 mmol, 1.0 eq) and 2-methyl-9-heptadecanol (8.3 g, 30.7 mmol, 2 eq) in dichloromethane (150 mL) was cooled to 0° C. and added EDC·HCl (17.6 g, 92.1 mmol, 3 eq) followed by 4-(dimethylamino)pyridin-1-ium (7.5 g, 61.4 mmol, 2 eq). The reaction mixture was stirred for 16 h at room temperature. TLC showed formation of new spot and starting material was consumed. The reaction mixture was quenched with brine solution and extract with DCM (3×0.5 L). The resulting organic layer was dried over sodium sulphate, filtered and concentrated under vacuum pressure to get the crude product. The crude product was purified under combi-flash chromatography by using 0-2% ethyl acetate in hexane. The desired fraction was collected and concentrated to get 8-methyl-1-octylnonyl 7-octenoate (8 g, 63.51%, Yield) as colour less liquid. ¹H-NMR (400 MHz, CDCl₃)-δ 5.84-5.74 (m, 1H), 5.02-4.83 (m, 3H), 2.28 (t, J=7.2 Hz, 2H), 2.05 (q, J=7.2 Hz, 2H), 1.62 (qu, J=7.2 Hz, 2H), 1.49-1.44 (m, 4H), 1.40-1.25 (m, 25H), 1.25-1.14 (m, 2H), 0.89-0.83 (m, 9H).

Step 8: Synthesis of 2-methylheptadecan-9-yl 6-(oxiran-2-yl)hexanoate

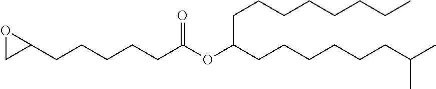 [SMILES: CCCCCCCCC(CCCCCCC(C)C)OC(=O)CCCCCC1CO1]

To a stirred solution of 8-methyl-1-octylnonyl 7-octenoate (7.7 g, 19.5 mmol, 1.0 eq) in dichloromethane (250 mL) was added 3-chlorobenzene-1-carboperoxoic acid (5.05 g, 29.3 mmol 1.5 eq) at 0° C. The resulting reaction mixture was stirred for 16 h at room temperature. Progress of reaction mass was monitored by ELSD/TLC (SM was consumed). The resulting reaction mixture was washed with cold aqueous sodium bicarbonate solution (100 mL). The resulting organic layer was dried over Na₂SO₄ and concentrated under reduce pressure. The crude was purified by flash column chromatography (SiO₂: 0-5% ethyl acetate in hexane) to give 8-methyl-1-octylnonyl 6-(2-oxiranyl)hexanoate (6 g, 74.86%, Yield) as a colourless liquid. ¹H-NMR (400 MHz, CDCl₃) −4.86 (qu, J=6.4 Hz, 1H), 2.89 (br, 1H), 2.74 (t, J=4.4 Hz, 1H), 2.46-2.44 (m, 1H), 2.29 (t, J=7.6 Hz, 2H), 1.68-1.62 (m, 2H), 1.55-1.44 (m, 7H), 1.43-1.31 (m, 2H), 1.30 (br, 22H), 1.11 (br, 2H), 0.91-0.87 (m, 9H).

Step 9: Synthesis of 2-methylheptadecan-9-yl (Z)-8-((2-((tert-butyldimethylsilyl)oxy)ethyl)(2-hydroxyoctadec-9-en-1-yl)amino)-7-hydroxyoctanoate

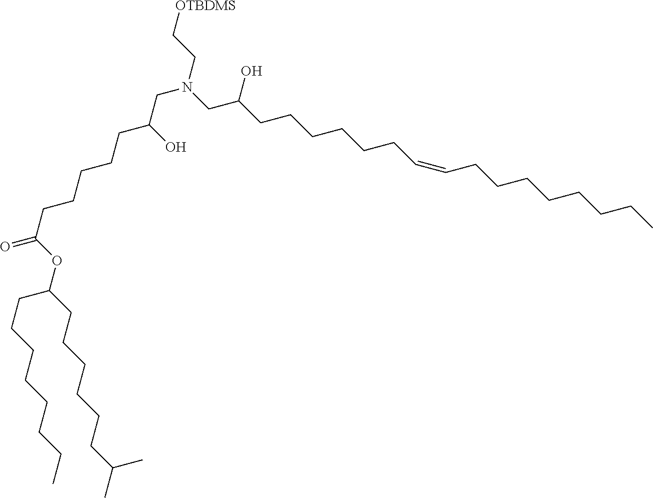 [SMILES: CCCCCCCC/C=C\CCCCCCC(O)CN(CCO[Si](C)(C)C(C)(C)C)CC(O)CCCCCC(=O)OC(CCCCCCCC)CCCCCCC(C)C]

To a stirred solution of (Z)-2-(tert-butyl)-2-methyl-3-oxa-6-aza-2-sila-15-tetracosen-8-ol (3.3 g, 7.47 mmol, 1.0 eq) in isopropanol (0.1 L) was added 8-methyl-1-octylnonyl 6-(2-oxiranyl)hexanoate (2.76 g, 6.72 mmol, 0.9 eq) at room temperature. The reaction was stirred at 90° C. for 16 h. The progress of reaction was monitored by TLC. Then solvent was evaporated to get crude compound. The crude compound was purified by column chromatography using silica gel (SiO₂: 0-20% methanol in dichloromethane) to afford 2-methylheptadecan-9-yl (Z)-8-((2-((tert-butyldimethylsilyl)oxy)ethyl)(2-hydroxyoctadec-9-en-1-yl)amino)-7-hydroxyoctanoate (3 g, 47.11%, Yield) as yellow viscous liquid. ELSD analysis: Purity 98.85%, Calculated C₅₂H₁₀₆NO₅Si, [M+H⁺]=852.78, Observed=852.65 (m/z, M+H⁺).

Step 10: Synthesis of 2-methylheptadecan-9-yl (Z)-6-(4-(2-((tert-butyldimethylsilyl)oxy)ethyl)-6-(hexadec-7-en-1-yl)morpholin-2-yl)hexanoate

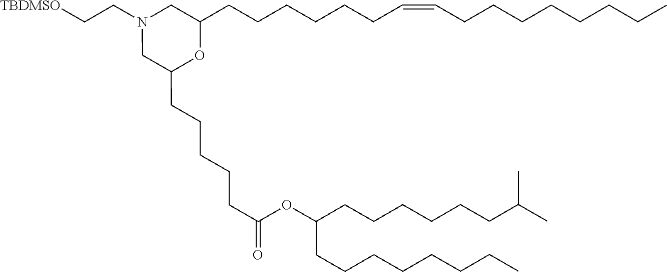 [SMILES: CCCCCCCC/C=C\CCCCCCC1CN(CCO[Si](C)(C)C(C)(C)C)CC(CCCCCC(=O)OC(CCCCCCCC)CCCCCCC(C)C)O1]

To a stirred solution of 8-methyl-1-octylnonyl 8-{[(Z)-2-hydroxy-9-octadecenyl]{2-[(tert-butyl)bis(methyl)siloxy]ethyl}amino}-7-hydroxyoctanoate (3.6 g, 4.22 mmol, 1.0 eq) in Toluene (40 mL) was added triphenylphosphine (1.33 g, 5.07 mmol 1.2 eq) portion wise. After 10 minutes N-{[(propan-2-yloxy)carbonyl]imino}(propan-2-yloxy)formamide (1.02 g, 5.07 mmol, 1.2 eq) was added dropwise to the reaction mixture. The reaction mixture was stirred at 40° C. for 16 hrs. The reaction was monitored by ELSD & TLC. After completion of reaction quenched with water (50 ml) and extracted with ethyl acetate (250 ml). Organic layer was dried over anhydrous sodium sulphate and concentrated under reduced vacuum. The crude was purified by column chromatography (0-10% Ethyl Acetate in Hexane) to giver 8-methyl-1-octylnonyl 6-{6-[(Z)-7-hexadecenyl]-4-{2-[(tert-butyl)bis(methyl)siloxy]ethyl}-2-morpholinyl}hexanoate (1.2 g, 34.05%, Yield) as colourless liquid. ELSD analysis: Purity 90.5%, Calculated C₅₂H₁₀₄NO₄Si, [M+H⁺]=834.77, Observed=834.60 (m/z, M+H⁺).

Step 11: Synthesis of 2-methylheptadecan-9-yl (Z)-6-(6-(hexadec-7-en-1-yl)-4-(2-hydroxyethyl)morpholin-2-yl)hexanoate

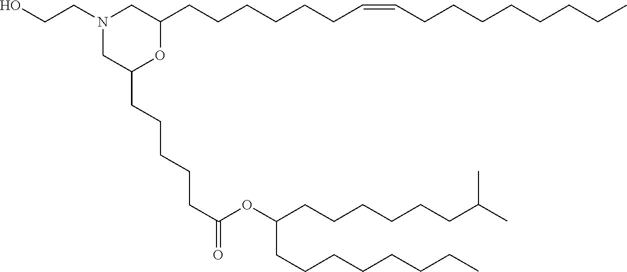 [SMILES: CCCCCCCC/C=C\CCCCCCC1CN(CCO)CC(CCCCCC(=O)OC(CCCCCCCC)CCCCCCC(C)C)O1]

To a stirred solution of 8-methyl-1-octylnonyl 6-{6-[(Z)-7-hexadecenyl]-4-{2-[(tert-butyl)bis(methyl)siloxy]ethyl}-2-morpholinyl}hexanoate (1.0 g. 1.2 mmol, 1.0 eq) in tetrahydrofuran (10 mL) was added hydrogen fluoride-pyridine (1/1) (594 mg, 5.99 mmol, 5 eq) at 0° C. The resulting reaction mixture was stirred for 16 h at room temperature. Progress of reaction mass was monitored by ELSD/TLC (SM was consumed). The resulting reaction mixture was quenched with cold aqueous sodium bicarbonate solution (20 mL) and extracted with ethyl acetate (3×25 mL). The resulting organic layer was dried over sodium sulphate and concentrated under reduce pressure. The crude was purified by flash column chromatography (SiO₂: 0-30% Ethyl acetate in Hexane) to obtain the desired produced 8-methyl-1-octylnonyl 6-{6-[(Z)-7-hexadecenyl]-4-(2-hydroxyethyl)-2-morpholinyl}hexanoate (250 mg, 28.89%, Yield) as colourless liquid. The compound (50 mg) was dissolved in dichloromethane (10 mL) and was filtered through hydrophobic PTFE syringe filter (25 mm×0.45 μm) using glass syringe to get desired 8-methyl-1-octylnonyl 6-{6-[(Z)-7-hexadecenyl]-4-(2-hydroxyethyl)-2-morpholinyl}hexanoate (35 mg) as a colourless liquid. ¹H-NMR (400 MHz, CDCl₃-d3): δ 5.38-5.33 (m, 2H), 4.89-4.82 (m, 1H), 3.66 (s, 2H), 3.54 (b, 2H), 2.83-2.75 (m, 2H), 2.58 (s, 2H), 2.27 (t, J=7.6 Hz, 2H), 2.05-2.00 (m, 4H), 1.90 (t, J=10.0 Hz, 2H), 1.66-1.58 (m, 3H), 1.55-1.47 (m, 9H), 1.43-1.25 (m, 46H), 1.14-1.12 (m, 2H), 0.89-0.85 (m, 12H). ELSD analysis: Purity 98.19%, Calculated C₄₆H₉₀NO₄=720.68, Observed=720.60 (m/z, M+H⁺).

Example 213. Synthesis of Compound 99:2-methylheptadecan-9-yl (Z)-6-(4-(2-((3-(dimethylamino)propanoyl)oxy)ethyl)-6-(hexadec-7-en-1-yl)morpholin-2-yl)hexanoate

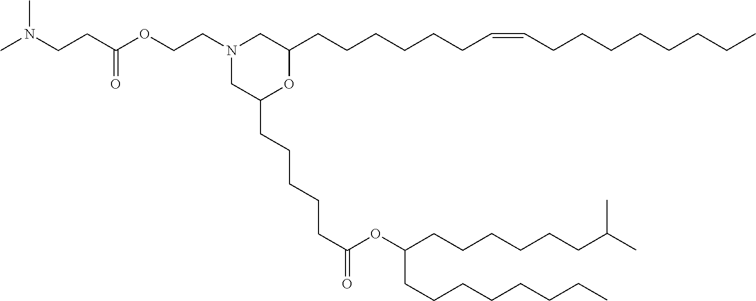 [SMILES: CCCCCCCC/C=C\CCCCCCC1CN(CCOC(=O)CCN(C)C)CC(CCCCCC(=O)OC(CCCCCCCC)CCCCCCC(C)C)O1]

To a stirred solution of 3-(dimethylamino) propionic acid (107 mg, 694 μmol, 4 eq) in dichloromethane (25 mL) was added {3-[cyano(ethyl)amino]propyl}dimethylazanium chloride (133 mg, 694 μmol, 4 eq) and N,N-dimethyl-4-pyridylamine (84.8 mg, 694 μmol, 4 eq). The reaction mixture was stirred for 40 min, then added 8-methyl-1-octylnonyl 6-{6-[(Z)-7-hexadecenyl]-4-(2-hydroxyethyl)-2-morpholinyl}hexanoate (125 mg, 174 μmol, 1.0 eq). The reaction mixture was stirred at r.t. for 16 h. The progress of reaction was monitored by TLC/ELSD. The reaction mixture was quenched with brine solution and extracted with DCM (10 mL×2). The combined organic layer was dried over sodium sulphate, filtered and concentrated to get the crude mixture. The crude was diluted with pentane and washed with acetonitrile (10 mL×3). Pentane layer was evaporated and distilled (temp. below 30° C.) to get 8-methyl-1-octylnonyl 6-{6-[(Z)-7-hexadecenyl]-4-{2-[3-(dimethylamino)propionoxy]ethyl}-2-morpholinyl}hexanoate (0.11 g) as colourless liquid. The compound (110 mg) was dissolved in dichloromethane (10 mL) and was filtered through hydrophobic PTFE syringe filter (25 mm×0.45 μm) using glass syringe to get 8-methyl-1-octylnonyl 6-{6-[(Z)-7-hexadecenyl]-4-{2-[3-(dimethylamino)propionoxy]ethyl}-2-morpholinyl}hexanoate (99 mg, 70.30%, Yield) as colourless liquid. ¹H-NMR (400 MHz, CDCl₃-d₃): δ 5.38-5.30 (m, 2H), 4.88-4.82 (m, 1H), 4.23-4.01 (m, 2H), 3.46-3.42 (m, 2H), 2.76-2.73 (m, 4H), 2.61-2.55 (m, 3H), 2.30-2.53 (m, 7H), 2.03-1.98 (m, 4H), 1.88-1.77 (m, 5H), 1.67-1.60 (m, 3H), 1.53-1.50 (m, 6H), 1.40-1.29 (m, 45H), 1.14-1.11 (m, 2H), 0.89-0.85 (m, 12H). ELSD analysis: Purity 95.60%, Calculated C₅₁H₉₉N₂O₅. [M+H]=819.75, Observed=819.60 (m/z, M+H⁺).

### Example 214. Synthesis of Compound 100

Step 1: Synthesis of oct-7-enoic acid

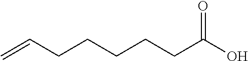

To a stirred solution of potassium tert-butoxide (113 g, 1.01 mol, 4.5 eq) in tetrahydrofuran (1 L, 12.3 mol) was added 8-bromooctanoic acid (50.0 g) at room temperature. The reaction mixture was stirred at 90° C. for 16 h. The progress of reaction was monitor by TLC. Reaction mass was diluted with EtOAc (1 L) and water (1 L), make pH acidic with 1N HCl and extracted with EtOAc (2×1 L). Combined organic layer was dried over Na₂SO₄ and concentrated under reduce pressure to obtain oct-7-enoic acid (30 g, 94.14%, Yield) as pale yellow liquid which was used as such for next step.

Step 2: Synthesis of heptadecan-9-yl oct-7-enoate

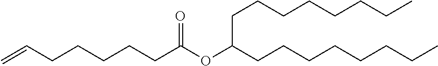 [SMILES: C=CCCCCCC(=O)OC(CCCCCCCC)CCCCCCCC]

A stirred solution of oct-7-enoic acid (20 g, 141 mmol, 1.0 eq) and heptadecan-9-ol (5 g, 19.5 mmol, 1.0 eq) in dichloromethane (500 mL) was cooled to 0° C. and added EDC·HCl (80.9 g, 422 mmol, 3 eq) followed by 4-(dimethylamino)pyridin-1-ium (34.4 g, 281 mmol, 2 eq). The reaction mixture was stirred for 16 h at room temperature. TLC showed formation of new spot and starting material was consumed. The reaction mixture was quenched with brine solution and extract with DCM (3×1 L). The resulting organic layer was dried over sodium sulphate, filtered and concentrated under vacuum pressure to get the crude product. The crude product was purified under combi-flash chromatography by using 0-2% ethyl acetate in hexane to get a 1-octylnonyl 7-octenoate (34 g, 63.51%, Yield) as colour less liquid. ¹H-NMR (400 MHz, CDCl₃-d3)−δ 5.84-5.74 (m, 1H), 5.01-4.92 (m, 2H), 4.86 (qu, J=6.0 Hz, 1H), 2.28 (t, J=7.2 Hz, 2H), 2.05 (q. J=6.8 Hz, 2H), 1.62 (qu, J=7.2 Hz, 2H), 1.49-1.44 (m, 4H), 1.40-1.25 (m, 28H), 0.89-0.83 (m, 6H).

Step 3: Synthesis of heptadecan-9-yl 6-(oxiran-2-yl)hexanoate

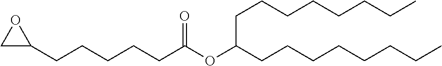 [SMILES: CCCCCCCCC(CCCCCCCC)OC(=O)CCCCCC1CO1]

To a stirred solution of 1-octylnonyl 7-octenoate (34 g, 89.3 mmol, 1.0 eq) in dichloromethane (500 mL), was added 3-chlorobenzene-1-carboperoxoic acid (23.1 g, 134 mmol, 1.5 eq) at 0° C. The resulting reaction mixture was stirred for 16 h at room temperature. Progress of reaction mass was monitored by ELSD/TLC (SM was consumed). The resulting reaction mixture was washed with cold aqueous sodium bicarbonate solution (500 mL). The resulting organic layer was dried over Na₂SO₄ and concentrated under reduce pressure. The crude was purified by flash column chromatography (SiO₂: 0-5% ethyl acetate in hexane), to give the desired 1-octylnonyl 6-(2-oxiranyl)hexanoate (30 g, 84.68%, Yield) as a colourless liquid. ¹H-NMR (400 MHz, CDCl₃) −4.88 (qu, J=6.4 Hz, 1H), 2.94-2.89 (br, 1H), 2.75-2.73 (t, J=7.6 Hz, 1H), 2.49-2.47 (m, 1H), 2.33 (t, J=7.6 Hz, 2H), 1.68-1.62 (m, 2H), 1.55-1.44 (m, 9H), 1.25 (br, 25H), 0.91-0.87 (m, 6H).

Step 4: Synthesis of di(heptadecan-9-yl) 8,8′-((2-((tert-butyldimethylsilyl)oxy)ethyl)azanediyl)bis(7-hydroxyoctanoate)

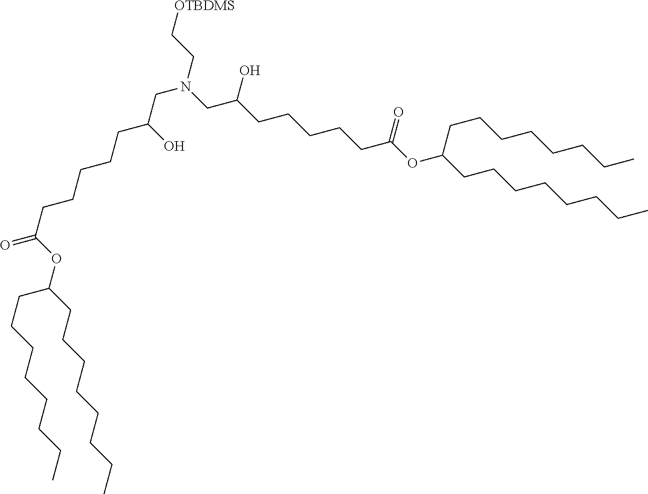 [SMILES: CCCCCCCCC(CCCCCCCC)OC(=O)CCCCCC(O)CN(CCO[Si](C)(C)C(C)(C)C)CC(O)CCCCCC(=O)OC(CCCCCCCC)CCCCCCCC]

To the stirred solution of 1-octylnonyl 6-(2-oxiranyl)hexanoate (10 g, 2.5 eq., 25.2 mmol) in isopropanol (40 mL, 523 mmol) was added 2-[(tert-butyl)bis(methyl)siloxy]ethylamine (1.77 g, 10.1 mmol) at room temperature. The reaction was allowed to stir at 90° C. for 16 h. Progress of reaction mass was monitored by ELSD/TLC (SM was consumed). The reaction mixture was concentrated to get crude compound. The crude compound was purified by column chromatography using silica gel to afford di(heptadecan-9-yl) 8,8′-((2-((tert-butyldimethylsilyl)oxy)ethyl)azanediyl)bis(7-hydroxyoctanoate) (5.5 g, 56.2%, Yield) as Yellow viscous compound. ELSD analysis: Purity 99.72%, Calculated C₅₈H₁₁₇NO₇Si, [M+H⁺]=968.86, Observed=968.45 (m/z, M+H⁺).

Step 5: Synthesis of di(heptadecan-9-yl) 6,6′-(4-(2-((tert-butyldimethylsilyl)oxy)ethyl)morpholine-2,6-diyl)dihexanoate

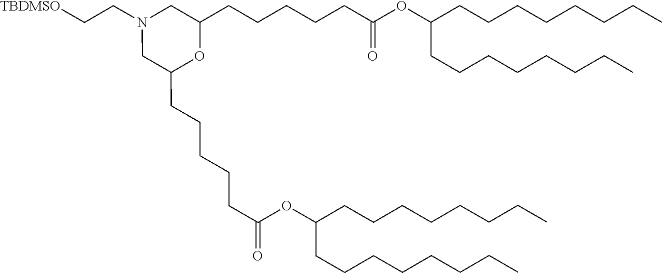 [SMILES: CCCCCCCCC(CCCCCCCC)OC(=O)CCCCCC1CN(CCO[Si](C)(C)C(C)(C)C)CC(CCCCCC(=O)OC(CCCCCCCC)CCCCCCCC)O1]

To a stirred solution of 1-octylnonyl 8-({2-[(tert-butyl)bis(methyl)siloxy]ethyl}[2-hydroxy-7-(1-octylnonyloxy carbonyl)heptyl]amino)-7-hydroxyoctanoate (1.5 g, 1.55 mmol, 1.0 eq) in toluene (20 mL), was added triphenylphosphine (487 mg, 1.86 mmol, 1.2 eq) portion wise. After 10 minutes N-{[(propan-2-yloxy)carbonyl]imino}(propan-2-yloxy)formamide (376 mg, 1.86 mmol, 1.2 eq) was added dropwise to the same reaction mixture. The reaction mixture was stirred at 40° C. for 16 hrs. The progress of reaction was monitored by ELSD & TLC. The reaction was quenched with water (50 ml) and extracted with ethyl acetate (250 ml). Organic layer was dried over anhydrous sodium sulphate and concentrated under reduced vacuum. The crude was purified by column chromatography (0-10% Ethyl Acetate in Hexane) to give di(heptadecan-9-yl)_{6,6}′-(4-(2-((tert-butyldimethylsilyl)oxy)ethyl)morpholine-2,6-diyl)dihexanoate (0.5 g, 33.97%, Yield) as colourless liquid. ELSD analysis: Purity 99.68%, Calculated C₅₈H₁₁₆NO₆Si, [M+H⁺]=950.85, Observed=950.50 (m/z, M+H⁺).

Step 6: Synthesis of di(heptadecan-9-yl) 6,6′-(4-(2-hydroxyethyl)morpholine-2,6-diyl)dihexanoate

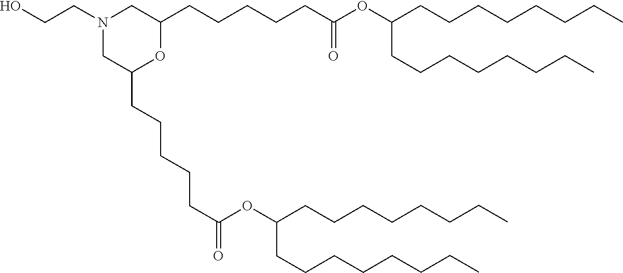 [SMILES: CCCCCCCCC(CCCCCCCC)OC(=O)CCCCCC1CN(CCO)CC(CCCCCC(=O)OC(CCCCCCCC)CCCCCCCC)O1]

To a stirred solution of 1-octylnonyl 6-(4-{2-[(tert-butyl)bis(methyl)siloxy]ethyl}-6-[5-(1-octylnonyloxycarbonyl)pentyl]-2-morpholinyl)hexanoate (550 mg, 579 μmol, 1.0 eq) in tetrahydrofuran (10 mL), was added hydrogen fluoride-pyridine (1/1)(287 mg. 2.89 mmol, 5 eq) at 0° C. The resulting reaction mixture was stirred for 16 h at room temperature. The progress of reaction mass was monitored by ELSD/TLC (SM was consumed). The resulting reaction mixture was quenched with cold aqueous sodium bicarbonate solution (20 mL) and extracted with ethyl acetate (3×25 mL). The resulting organic layer was dried over sodium sulphate and concentrated under reduce pressure The crude was purified by flash column chromatography (SiO₂: 0-30% Ethyl acetate in Hexane) to obtain 1-octylnonyl 6-{4-(2-hydroxyethyl)-6-[5-(1-octylnonyloxy carbonyl)pentyl]-2-morpholinyl}hexanoate (0.3 g, 62%, Yield) as colourless liquid. The compound (120 mg) which was obtained after column chromatography was dissolved in dichloromethane (10 mL) and was filtered through hydrophobic PTFE syringe filter (25 mm×0.45 μm) using glass syringe to get desired 6-{4-(2-hydroxyethyl)-6-[5-(1-octylnonyloxycarbonyl)pentyl]-2-morpholinyl}hexanoate (0.1 g) as a colourless liquid. 1H NMR (400 MHz, CDCl₃-d3): δ 4.87-4.82 (m, 2H), 4.40-4.29 (t, J=5.2 Hz, 2H), 3.46-3.342 (m, 2H), 2.73 (d, J=10.4 Hz, 2H), 2.51 (t, J=5.2 Hz, 2H), 2.27 (t, J=7.2 Hz, 4H), 1.82 (t, J=10.8 Hz, 2H), 1.66-1.58 (m, 6H), 1.52-1.49 (m, 12H), 1.40-1.31 (m, 12H), 1.25 (b, 42H), 0.87 (t, J=6.8 Hz, 12H). ELSD analysis: Purity 99.13%, Calculated C₅₂H₁₀₂NO₆=836.76, Observed=836.85 (m/z, M+H⁺).

Step 7: Synthesis of di(heptadecan-9-yl) 6,6′-(4-(2-((3-(dimethylamino)propanoyl)oxy)ethyl)morpholine-2,6-diyl)dihexanoate

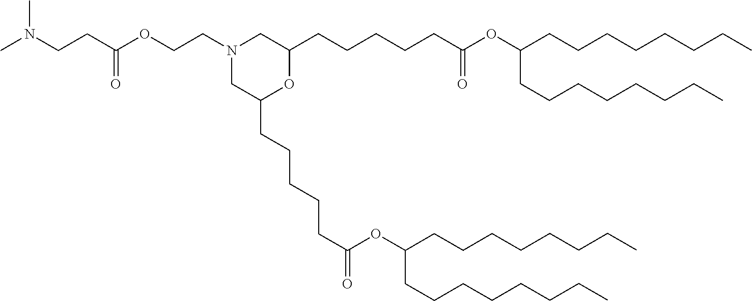 [SMILES: CCCCCCCCC(CCCCCCCC)OC(=O)CCCCCC1CN(CCOC(=O)CCN(C)C)CC(CCCCCC(=O)OC(CCCCCCCC)CCCCCCCC)O1]

To a stirred solution of 3-(dimethylamino) propionic acid-hydrogen chloride (1/1)(176 mg, 1.15 mmol, 8 eq) in dichloromethane (10 ml) was added {3-[cyano(ethyl)amino]propyl}dimethylazanium chloride (110 mg, 574 μmol, 4 eq) and 4-(dimethylamino)pyridin-1-ium (70.7 mg, 574 μmol, 4 eq). The reaction mixture was stirred for 40 min, then added 1-octylnonyl 6-{4-(2-hydroxyethyl)-6-[5-(1-octylnonyloxycarbonyl)pentyl]-2-morpholinyl}hexanoate (120 mg, 143 μmol, 1.0 eq). The reaction mixture was stirred at r.t. for 16 h. The progress of reaction was monitored by TLC/ELSD. The reaction mixture was quenched with brine solution and extracted with DCM (10 mL×2). The combined organic layer was dried over sodium sulphate, filtered and concentrated to get the crude mixture. The crude was diluted with pentane and washed with acetonitrile (10 mL×3). Pentane layer was evaporated and distilled (temp. below 30° C.) to get di(heptadecan-9-yl) 6,6′-(4-(2-((3-(dimethylamino)propanoyl)oxy)ethyl)morpholine-2,6-diyl)dihexanoate (116 mg) as colourless liquid. The compound (116 mg) was dissolved in dichloromethane (10 mL) and was filtered through hydrophobic PTFE syringe filter (25 mm×0.45 μm) using glass syringe to get di(heptadecan-9-yl) 6,6′-(4-(2-((3-(dimethylamino)propanoyl)oxy)ethyl)morpholine-2,6-diyl)dihexanoate (112 mg, 83%, Yield) as a colourless liquid. ¹H-NMR (400 MHz, CDCl₃-d₃): δ 4.88-4.82 (t, J=6.0 Hz, 2H), 4.21 (t, J=6.0 Hz, 2H), 3.48-3.42 (m, 2H), 2.74 (d, J=10.8 Hz, 2H), 2.62-2.57 (m, 4H), 2.50-2.47 (m, 2H), 2.27 (t, J=7.2 Hz, 3H), 2.23 (s, 6H), 1.79 (t, J=10.4 Hz, 2H), 1.65-1.62 (m, 3H), 1.50-1.42 (m, 12H), 1.39-1.25 (br, 58H), 0.88-0.85 (m, 12H). ELSD analysis: Purity 99.78%, Calculated C₅₇H₁₁₁N₂O₇, [M+H⁺]=935.83, Observed=935.80 (m/z, M+H⁺).

### Example 215. Synthesis of Compound 101

Step 1: Synthesis of nonyl oct-7-enoate

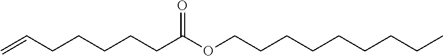 [SMILES: C=CCCCCCC(=O)OCCCCCCCCC]

A stirred solution of oct-7-enoic acid (20 g, 141 mmol, 1.0 eq) and 1-nonanol (18.9 g, 131 mmol, 1.0 eq) in dichloromethane (400 mL) was cooled to 0° C. and added {3-[cyano(ethyl)amino]propyl}dimethylazanium chloride (75.2 g, 392 mmol, 3 eq) followed by 4-(dimethylamino)pyridin-1-ium (16 g, 131 mmol, 1 eq). The reaction mixture was stirred for 16 h at room temperature. TLC showed formation of new spot and starting material was consumed. The reaction mixture was quenched with brine solution and extract with DCM (3×1 L). The resulting organic layer was dried over sodium sulphate, filtered and concentrated under vacuum pressure to get the crude product. The crude product was purified under combi-flash chromatography by using 0-2% ethyl acetate in hexane to get a nonyl 7-octenoate (18.9 g, 53.83%, Yield) as colour less liquid. ¹H-NMR (400 MHz, CDCl₃-d3) −δ 5.86-5.78 (m, 1H), 5.03-4.94 (m, 2H), 4.07 (t, J=6.8 Hz, 2H), 2.31 (t, J=7.6 Hz, 2H), 2.09-2.04 (m, 2H), 1.68 (m, 4H), 1.46-1.29 (m, 16H), 0.91-0.88 (m, 3H).

Step 2: Synthesis of nonyl 6-(oxiran-2-yl)hexanoate

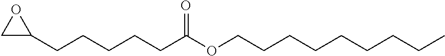 [SMILES: CCCCCCCCCOC(=O)CCCCCC1CO1]

To a stirred solution of nonyl 7-octenoate (18.9 g, 70.4 mmol, 1.0 eq) in dichloromethane (500 mL), 3-chlorobenzene-1-carboperoxoic acid (18.2 g, 106 mmol, 1.5 eq) was added at 0° C. The resulting reaction mixture was stirred for 16 h at room temperature. Progress of reaction mass was monitored by ELSD/TLC (SM was consumed). The resulting reaction mixture was washed with cold aqueous sodium bicarbonate solution (500 mL). The resulting organic layer was dried over Na₂SO₄ and concentrated under reduce pressure, and the crude was purified by flash column chromatography (SiO₂: 0-5% ethyl acetate in hexane), to give the desired nonyl 6-(2-oxiranyl)hexanoate (11 g, 54.93%, Yield) as a yellow colorless liquid. ELSD analysis: Purity 97.07%, Calculated C17H33O3, [M+H+]=285.24, Observed=285.30 (m/z, M+H+).

Step 3: Synthesis of nonyl 8-((2-((tert-butyldimethylsilyl)oxy)ethyl)amino)-7-hydroxyoctanoate

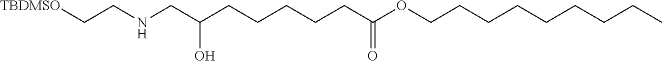 [SMILES: CCCCCCCCCOC(=O)CCCCCC(O)CNCCO[Si](C)(C)C(C)(C)C]

To a stirred solution of nonyl 6-(2-oxiranyl)hexanoate (13.6 g, 47.8 mmol, 1.0 eq) in isopropanol (0.2 L, 2.62 mol) was added 2-[(tert-butyl)bis(methyl)siloxy]ethylamine (8.38 g, 47.8 mmol, 1.0 eq) at room temperature. The reaction was stirred at 40° C. for 16 h. Progress of reaction mass was monitored by ELSD/TLC (SM was consumed). The reaction mixture was concentrated under vacuum to get crude compound. The crude compound was purified by column chromatography using silica gel to afford nonyl 8-((2-((tert-butyldimethylsilyl)oxy)ethyl)amino)-7-hydroxyoctanoate (5.0 g, 56.2%, Yield) as colorless liquid. ELSD analysis: Purity 99.83%, Calculated C25H54NO4Si, [M+H]=460.37, Observed=460.35 (m/z, M+H+).

Step 4: Synthesis of heptadecan-9-yl 8-((2-((tert-butyldimethylsilyl)oxy)ethyl)(2-hydroxy-8-(nonyloxy)-8-oxooctyl)amino)-7-hydroxyoctanoate

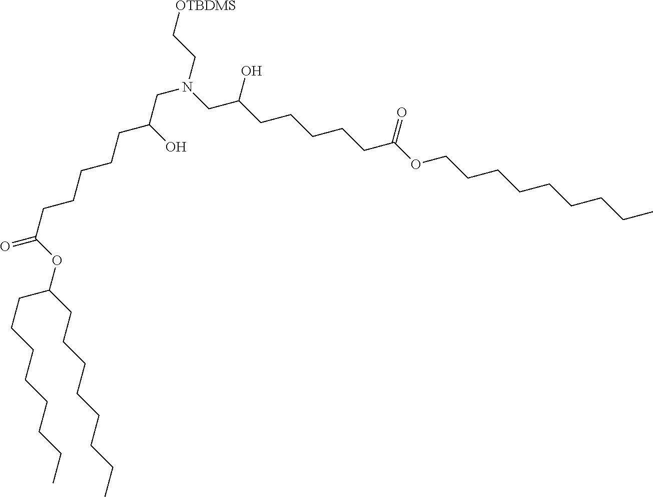 [SMILES: CCCCCCCCCOC(=O)CCCCCC(O)CN(CCO[Si](C)(C)C(C)(C)C)CC(O)CCCCCC(=O)OC(CCCCCCCC)CCCCCCCC]

To a stirred solution of nonyl 8-((2-((tert-butyldimethylsilyl)oxy)ethyl)amino)-7-hydroxyoctanoate (5 g, 10.9 mmol, 1.0 eq) in isopropanol (100 mL) was added 1-octylnonyl 6-(2-oxiranyl)hexanoate (4.31 g, 10.9 mmol, 1.0 eq) at room temperature. The reaction was stirred at 90° C. for 16 h. Progress of reaction mass was monitored by ELSD/TLC (SM was consumed). The reaction mixture was concentrated under vacuum to get. Then solvent was evaporated to get crude compound. The crude compound was purified by column chromatography using silica gel (SiO₂: 0-20% methanol in dichloromethane) to afford 1-octylnonyl 8-({2-[(tert-butyl)bis(methyl)siloxy]ethyl}[2-hydroxy-7-(nonyloxycarbonyl)heptyl]amino)-7-hydroxyoctanoate (6.2 g, 66.57%, Yield) as yellow viscous liquid. ELSD analysis: Purity 98.26%, Calculated C50H102NO7Si, [M+H]=856.73, Observed=856.40 (m/z, M+H+).

Step 5: Synthesis of heptadecan-9-yl 6-(4-(2-((tert-butyldimethylsilyl)oxy)ethyl)-6-(6-(nonyloxy)-6-oxohexyl)morpholin-2-yl)hexanoate

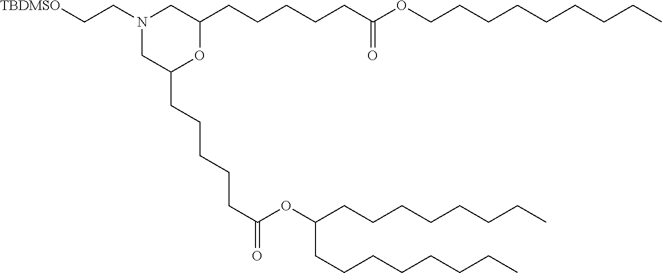 [SMILES: CCCCCCCCCOC(=O)CCCCCC1CN(CCO[Si](C)(C)C(C)(C)C)CC(CCCCCC(=O)OC(CCCCCCCC)CCCCCCCC)O1]

To a stirred solution of 1-octylnonyl 8-({2-[(tert-butyl)bis(methyl)siloxy]ethyl}[2-hydroxy-7-(nonyloxycarbonyl)heptyl]amino)-7-hydroxyoctanoate (2 g, 2.34 mmol, 1.0 eq) in toluene (40 mL) was added triphenylphosphine (735 mg, 2.8 mmol, 1.2 eq) portion wise. After 10 minutes N-{[(propan-2-yloxy)carbonyl]imino}(propan-2-yloxy)formamide (567 mg, 2.8 mmol, 1.2 eq) was added dropwise to the reaction mixture. The reaction mixture was stirred at 40° C. for 16 hrs. The reaction was monitored by ELSD & TLC. After completion of reaction quenched with water (50 ml) and extracted with ethyl acetate (250 ml). Organic layer was dried over anhydrous sodium sulphate and concentrated under reduced vacuum. The crude was purified by column chromatography (0-10% Ethyl Acetate in Hexane) to give 1-octylnonyl 6-(4-{2-[(tert-butyl)bis(methyl)siloxy]ethyl}-6-[5-(nonyloxycarbonyl)pentyl]-2-morpholinyl) hexanoate (0.4 g, 20.43%, Yield) as colourless liquid. ELSD analysis: Purity 96.57%, Calculated C₅₀H₁₀₀NO₆Si, [M+H⁺]=838.72, Observed=838.50 (m/z, M+H⁺).

Step 6: Synthesis of heptadecan-9-yl 6-(4-(2-hydroxyethyl)-6-(6-(nonyloxy)-6-oxohexyl)morpholin-2-yl)hexanoate

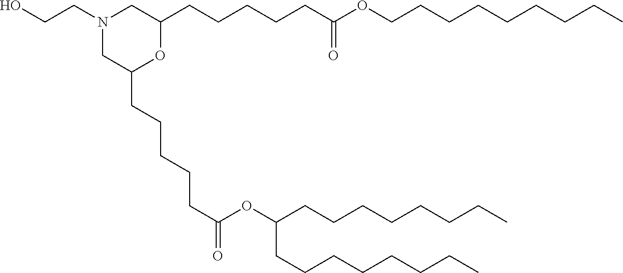 [SMILES: CCCCCCCCCOC(=O)CCCCCC1CN(CCO)CC(CCCCCC(=O)OC(CCCCCCCC)CCCCCCCC)O1]

To a stirred solution of 1-octylnonyl 6-(4-{2-[(tert-butyl)bis(methyl)siloxy]ethyl}-6-[5-(nonyloxycarbonyl)pentyl]-2-morpholinyl)hexanoate (480 mg, 572 μmol, 1.0 eq) in tetrahydrofuran (10 mL) was added hydrogen fluoride-pyridine (1/1) (284 mg, 2.86 mmol, 5 eq) at 0° C. The resulting reaction mixture was stirred for 16 h at room temperature. Progress of reaction mass was monitored by ELSD/TLC (SM was consumed). The resulting reaction mixture was quenched with cold aqueous sodium bicarbonate solution (20 mL) and extracted with ethyl acetate (3×25 mL). The resulting organic layer was dried over sodium sulphate and concentrated under reduce pressure. The crude was purified by flash column chromatography (SiO₂: 0-30% Ethyl acetate in Hexane), to obtain the desired produced heptadecan-9-yl 6-(4-(2-hydroxyethyl)-6-(6-(nonyloxy)-6-oxohexyl)morpholin-2-yl)hexanoate (0.30 g, 72.32%, Yield) as colourless liquid. The compound (135 mg) which was obtained after column chromatography was dissolved in dichloromethane (10 mL) and was filtered through hydrophobic PTFE syringe filter (25 mm×0.45 μm) using glass syringe to get desired heptadecan-9-yl 6-{4-(2-hydroxyethyl)-6-[5-(1-octylnonyloxycarbonyl)pentyl]-2-morpholinyl}hexanoate (0.125 g) as a colourless liquid. 1H NMR (400 MHz, CDCl₃-d3): δ 4.89-4.82 (m, 1H), 4.05 (t, J=6.8 Hz, 2H), 3.71-3.68 (b, 2H), 3.58 (b, 2H), 2.88 (b, 2H), 2.62 (b, 2H), 2.30-2.25 (m, 4H), 1.98-1.92 (m, 2H), 1.66-1.59 (m, 6H), 1.55-1.43 (m, 8H), 1.40-1.25 (m, 44H), 0.89-0.85 (m, 9H). ELSD analysis: Purity 99.82%, Calculated C44H86NO6=724.64, Observed=724.60 (m/z, M+H+).

Step 7: Synthesis of heptadecan-9-yl 6-(4-(2-((3-(dimethylamino)propanoyl)oxy)ethyl)-6-(6-(nonyloxy)-6-oxohexyl)morpholin-2-yl)hexanoate

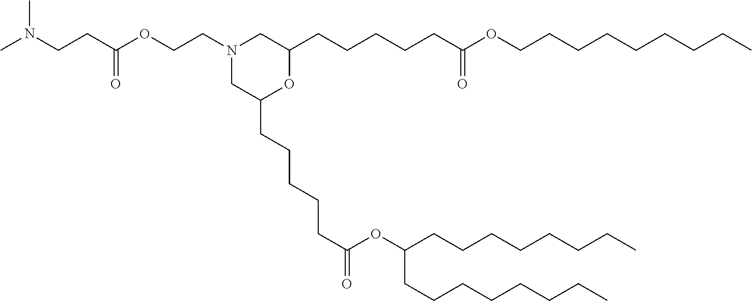 [SMILES: CCCCCCCCCOC(=O)CCCCCC1CN(CCOC(=O)CCN(C)C)CC(CCCCCC(=O)OC(CCCCCCCC)CCCCCCCC)O1]

To a stirred solution of 3-(dimethylamino)propionic acid—hydrogen chloride (1/1) (221 mg, 1.15 mmol, 8 eq) in dichloromethane (15 ml), was added {3-[cyano(ethyl)amino]propyl}dimethylazanium chloride (138 mg, 718 μmol, 4 eq) and 4-(dimethylamino)pyridin-1-ium (88.5 mg, 718 μmol, 4 eq). The reaction mixture was stirred for 40 min, then added heptadecan-9-yl 6-{4-(2-hydroxyethyl)-6-[5-(1-octylnonyl oxycarbonyl)pentyl]-2-morpholinyl}hexanoate (130 mg, 180 μmol, 1.0 eq). The reaction mixture was stirred at r.t. for 16 h. The progress of reaction was monitored by TLC/ELSD. The reaction mixture was quenched with brine solution and extracted with DCM (10 mL×2). The combined organic layer was dried over sodium sulphate, filtered and concentrated to get the crude mixture. The crude was diluted with pentane and washed with acetonitrile (10 mL×3). Pentane layer was distilled (temp. below 30° C.) under vacuum to get heptadecan-9-yl 6-(4-(2-((3-(dimethylamino)propanoyl)oxy)ethyl)-6-(6-(nonyloxy)-6-oxohexyl)morpholin-2-yl)hexanoate (130 mg) as colourless liquid. The compound (130 mg) was dissolved in dichloromethane (10 mL) and was filtered through hydrophobic PTFE syringe filter (25 mm×0.45 μm) using glass syringe to get heptadecan-9-yl 6-(4-(2-((3-(dimethylamino) propanoyl)oxy)ethyl)-6-(6-(nonyloxy)-6-oxohexyl)morpholin-2-yl)hexanoate (108 mg, 73%, Yield) as colourless liquid. ¹H-NMR (400 MHz, CDCl₃-d₃): δ 4.85 (qu, J=6.4 Hz, 1H), 4.21 (t, J=6.0 Hz, 2H), 4.04 (t, J=6.0 Hz, 2H), 3.45-3.42 (m, 2H), 2.74 (d, J=10.8 Hz, 2H), 2.64-2.57 (m, 4H), 2.50-2.47 (m, 2H), 2.30-2.23 (m, 9H), 1.79 (t, J=10.4 Hz, 2H), 1.67-1.60 (m, 8H), 1.50-1.42 (m, 7H), 1.37-1.25 (br, 44H), 0.88-0.85 (m, 9H). ELSD analysis: Purity 98.36%, Calculated C₄₉H₉₅N₂O₇, [M+H]=823.71, Observed=823.65 (m/z, M+H⁺).

### Example 216. Multi-dose Tolerability Screening of Additional Lipids

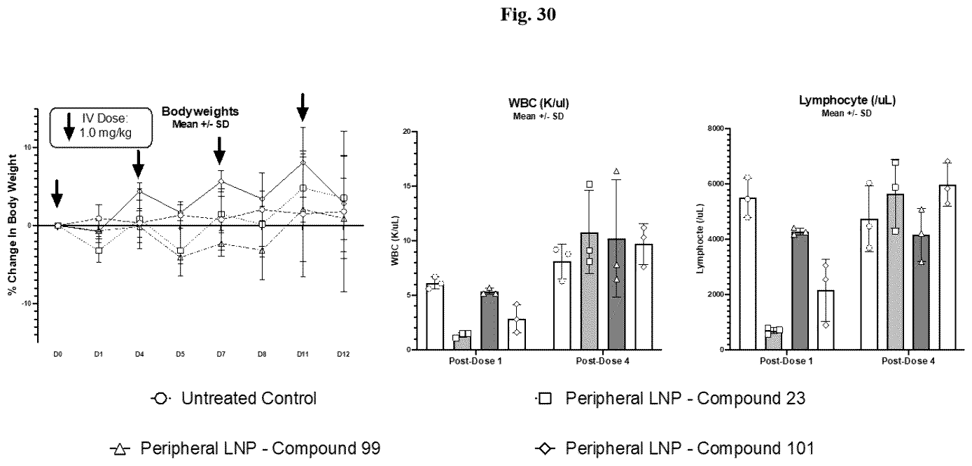

NT-beta-Actin mRNA LNPs with different ionizable lipids were administered IV to C57BL/6 mice, inoculated with MC38.K cells, via the tail vein for a total of four doses (twice a week for two consecutive weeks) at a concentration of 1.0 mg/kg. Bodyweights were monitored and blood was collected pre-dose, 24 hours post first dose, 24 hours post fourth dose, and analyzed for cytokines, blood chemistry, and liver/spleen weights. Compounds 23, 99 and 101 were formulated using a peripheral process method. C57BL/6 mice were inoculated with MC38.K cells. Once tumors had grown, 1.0 mg/kg (Beta-Actin mRNA) of each LNP was intravenously administered twice a week for two weeks. At the duration of the study, all mice survived. It was observed that Morpholino iLipids (Compound 99 and 101) showed less of a reduction in white blood cells and lymphocytes in the blood in addition to showing less adverse clinical observations compared to Compound 23 LNPs. Morpholino iLipids show improved tolerability. N=6 per group for bodyweights, N=3 for blood analysis. Mean+/−SD. The results are shown in FIG. 30.

Example 217. Screening Additional Lipids's Biodistribution via In Vivo Administration of Firefly Luciferase mRNA LNPs

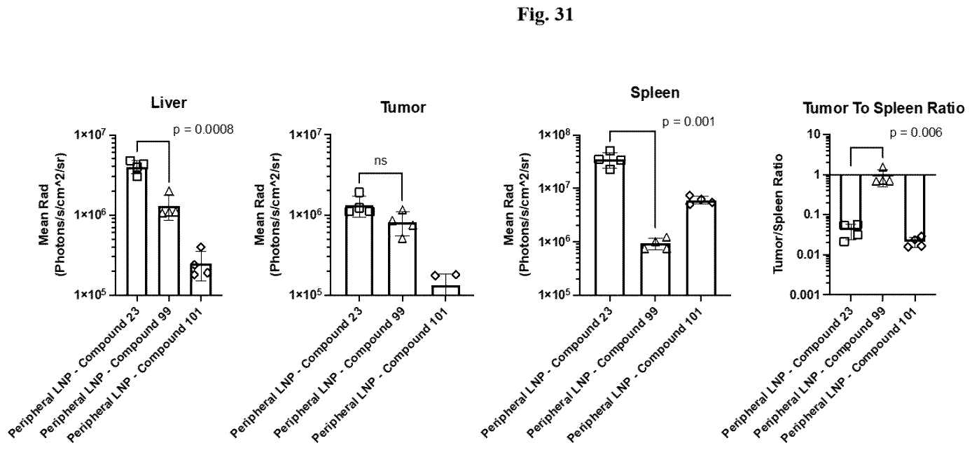

Fluc mRNA LNPs were administered to Albino B6 mice, inoculated with MC38.K cells, via the tail vein at 1.0 mg/kg. Eighteen hours later, bioluminescence was measured using an IVIS Spectrum imager. Mice were treated with 150 mg/kg luciferin substrate administered via intraperitoneal injection. Afterwards, mice were euthanized, and the lung, liver, spleen, and tumor were harvested for ex vivo imaging. All procedures were conducted according to the approved IACUC guidelines. N=5 per group. Data analysis was performed in Graphpad Prism 10. Values are reported as mean+/−standard deviation, unless specified otherwise. Ex-Vivo luminescence of liver, tumor, and spleen organs after 1.0 mg/kg IV injection of Peripheral LNPs. N=4 mice per group. (Mean±SD, n=4/group). Compound 99 shows nearly equivalent expression in solid tumor tissue while showing significantly less expression in liver and spleen. The results are shown in FIG. 31.

What is claimed is:

1. A compound of Formula I′: 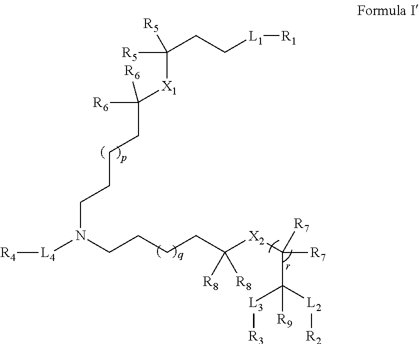
or its N-oxide, or a pharmaceutically acceptable salt thereof,
wherein:
L₁ is substituted or unsubstituted linear —C₃₋₁₂ alkylene or —C₃₋₁₂ alkenylalkylene;
L₂ is substituted or unsubstituted linear —C₄₋₁₂ alkylene or —C₃₋₁₂ alkenylalkylene;
L₃ is substituted or unsubstituted linear —C₄₋₁₂ alkylene or —C₃₋₁₂ alkenylalkylene;
wherein if L₁, L₂, or L₃ is substituted, then L₁, L₂, or L₃ is substituted with 1-5 substituents selected from the group consisting of halogen, deuterium, —CN, linear or branched C₁₋₁₀ alkyl, linear or branched C₁₋₁₀ heteroalkyl, C₁₋₂ haloalkyl, —C(═O)R¹⁰, —C(═O)N(R¹⁰)₂, —OR¹⁰, —N(R¹⁰)₂, substituted or unsubstituted C₃₋₆ cycloalkyl, and substituted or unsubstituted 3- to 10-membered heterocycloalkyl; wherein each substituted C₃₋₆ cycloalkyl and substituted 3- to 10-membered heterocycloalkyl is substituted with 1-5 R¹¹;
L₄ is substituted or unsubstituted —C₁₋₂₄ alkylene or —C₃₋₂₄ alkenylalkylene; wherein L₄ is optionally substituted with 1 to 10 R¹¹;
X₁ is a covalent bond, —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—;
X₂ is a covalent bond, —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—;
each R₁, R₂, and R₃, is independently hydrogen, deuterium, halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl;
provided that at least one of R₁, R₂, and R₃ is not hydrogen or deuterium;
or when each R₁, R₂, and R₃ is hydrogen, or deuterium, then at least one of L₁, L₂, L₃ is substituted with 1-5 substituents, and at least one of the substituents is not a linear C₁₋₁₀ alkyl substituted at a terminal carbon, or a deuterium;
R₄ is halogen, —OH, —OR¹⁰, —O—C(═O)—R¹⁰, —C(═O)—OR¹⁰, —O—C(═O)—OR¹⁰—, —C(═O)—O—C(═O)—R¹⁰, —O—C(═O)—C₀₋₉ alkylene-R¹¹, —(O—C₀₋₉alkylene-)ₙR¹¹, —O—C(═O)—C₀₋₉ alkylene-N(R¹⁰)₂, —N(R¹⁰)₂, or —C(═O)—C₀₋₉ alkylene-N(R¹⁰)₂;
each R₅, R₆, R₇, R₈, or R₉ is independently hydrogen, deuterium, halogen, —CN, —OR¹⁰, —N(R¹⁰)₂, —O—C(═O)—R¹⁰, C₁₋₁₀ alkyl, C₁₋₁₀ heteroalkyl, C₃₋₁₀ cycloalkyl, C₃₋₁₀ heterocycloalkyl, C₂₋₆ alkenyl, or C₂₋₆ alkynyl;
each R¹⁰ is independently hydrogen, C₁₋₁₀ alkyl, C₁₋₁₀ heteroalkyl, C₁₋₁₀ haloalkyl, C₃₋₁₀ cycloalkyl, C₃₋₁₀ heterocycloalkyl, C₂₋₆ alkenyl, or C₂₋₆ alkynyl;
each R¹¹ is independently hydrogen, deuterium, halogen, —CN, C₁₋₁₀ alkyl, C₁₋₁₀ heteroalkyl, C₁₋₁₀ haloalkyl, C₃₋₁₀ cycloalkyl, C₃₋₁₀ heterocycloalkyl, C₂₋₆ alkenyl, C₂₋₆ alkynyl, —C(O)OH, —C(O)—O—C₁₋₆ alkyl, —C(O)—C₁₋₆ alkyl, —C(O)NH₂, —C(O)NH(C₁₋₆ alkyl), —C(O)N(C₁₋₆ alkyl)₂, —NH₂, —NH(C₁₋₆ alkyl), —N(C₁₋₆ alkyl)₂, —OH, —O—C₁₋₆ alkyl, —SH, —S(C₁₋₆ alkyl), —S(O)(C₁₋₆ alkyl), —S(O)₂(C₁₋₆ alkyl), or —S(O)₂NH(C₁₋₆ alkyl);
n is an integer from 0-20;
p is 0, 1, 2, 3, 4, or 5;
q is 0, 1, 2, 3, 4, or 5; and
r is 0 or 1.

2. The compound of claim 1, wherein r is 0.

3. The compound of claim 1 or 2, wherein X₁ is —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O— and X₂ is —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O.

4. A compound of Formula I: 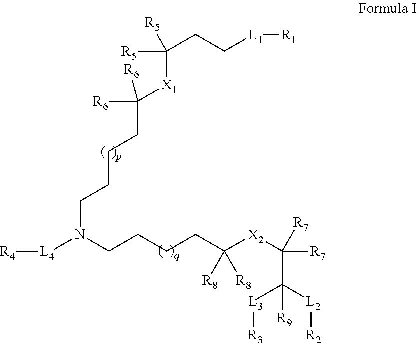
or its N-oxide, or a pharmaceutically acceptable salt thereof,
wherein:
L₁ is substituted or unsubstituted linear —C₃₋₁₂ alkylene or —C₃₋₁₂ alkenylalkylene;
L₂ is substituted or unsubstituted linear —C₄₋₁₂ alkylene or —C₃₋₁₂ alkenylalkylene;
L₃ is substituted or unsubstituted linear —C₄₋₁₂ alkylene or —C₃₋₁₂ alkenylalkylene;
wherein if L₁, L₂, or L₃ is substituted, then L₁, L₂, or L₃ is substituted with 1-5 substituents selected from the group consisting of halogen, deuterium, —CN, linear or branched C₁₋₁₀ alkyl, linear or branched C₁₋₁₀ heteroalkyl, C₁₋₂ haloalkyl, —C(═O)R¹⁰, —C(═O)N(R¹⁰)₂, —OR¹⁰, —N(R¹⁰)₂, substituted or unsubstituted C₃₋₆ cycloalkyl, and substituted or unsubstituted 3- to 10-membered heterocycloalkyl; wherein each substituted C₃₋₆ cycloalkyl and substituted 3- to 10-membered heterocycloalkyl is substituted with 1-5 R¹¹;
L₄ is substituted or unsubstituted —C₁₋₂₄ alkylene or —C₃₋₂₄ alkenylalkylene; wherein L₄ is optionally substituted with 1 to 10 R¹¹;
X₁ is —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—;
X₂ is —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—;
each R₁, R₂, and R₃, is independently hydrogen, deuterium, halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl;
provided that at least one of R₁, R₂, and R₃ is not hydrogen or deuterium;
or when each R₁, R₂, and R₃ is hydrogen, or deuterium, then at least one of L₁, L₂, L₃ is substituted with 1-5 substituents, and at least one of the substituents is not a linear C₁₋₁₀ alkyl substituted at a terminal carbon, or a deuterium;
R₄ is —OH, —OR¹⁰, —O—C(═O)—R¹⁰, —C(═O)—OR¹⁰, —O—C(═O)—OR¹⁰—, —C(═O)—O—C(═O)—R¹⁰, —O—C(═O)—C₀₋₉ alkylene-R¹¹, —(O—C₀₋₉ alkylene-)ₙR¹¹, —O—C(═O)—C₀₋₉ alkylene-N(R¹⁰)₂, —N(R¹⁰)₂, or —C(═O)—C₀₋₉ alkylene-N(R¹⁰)₂;
each R₅, R₆, R₇, R₈, or R₉ is independently hydrogen, deuterium, halogen, —CN, —OR¹⁰, —N(R¹⁰)₂, —O—C(═O)—R¹⁰, C₁₋₁₀ alkyl, C₁₋₁₀ heteroalkyl, C₃₋₁₀ cycloalkyl, C₃₋₁₀ heterocycloalkyl, C₂₋₆ alkenyl, or C₂₋₆ alkynyl;
each R¹⁰ is independently hydrogen, C₁₋₁₀ alkyl, C₁₋₁₀ heteroalkyl, C₁₋₁₀ haloalkyl, C₃₋₁₀ cycloalkyl, C₃₋₁₀ heterocycloalkyl, C₂₋₆ alkenyl, or C₂₋₆ alkynyl;
each R¹¹ is independently hydrogen, deuterium, halogen, —CN, C₁₋₁₀ alkyl, C₁₋₁₀ heteroalkyl, C₁₋₁₀ haloalkyl, C₃₋₁₀ cycloalkyl, C₃₋₁₀ heterocycloalkyl, C₂₋₆ alkenyl, C₂₋₆ alkynyl, —C(O)OH, —C(O)—O—C₁₋₆ alkyl, —C(O)—C₁₋₆ alkyl, —C(O)NH₂, —C(O)NH(C₁₋₆ alkyl), —C(O)N(C₁₋₆ alkyl)₂, —NH₂, —NH(C₁₋₆ alkyl), —N(C₁₋₆ alkyl)₂, —OH, —O—C₁₋₆ alkyl, —SH, —S(C₁₋₆ alkyl), —S(O)(C₁₋₆ alkyl), —S(O)₂(C₁₋₆ alkyl), or —S(O)₂NH(C₁₋₆ alkyl);
n is an integer from 0-20;
p is 0, 1, 2, 3, 4, or 5; and
q is 0, 1, 2, 3, 4, or 5.

5. The compound of any one of claims 1-4, wherein each R₅, R₆, R₇, R₈, or R₉ is independently hydrogen, deuterium, halogen, —CN, —OR¹⁰, or C₁₋₁₀ alkyl.

6. The compound of any one of claims 1-4, wherein each R₅, R₆, R₇, R₈, or R₉ is independently hydrogen, deuterium, halogen, methyl, ethyl, or isopropyl.

7. The compound of any one of claims 1-6, having the structure of Formula II: 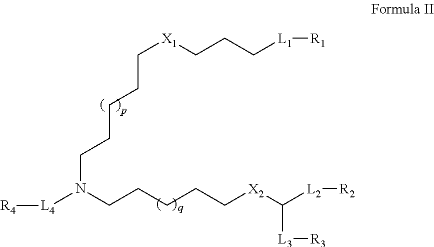
or its N-oxide, or a pharmaceutically acceptable salt thereof, wherein:
L₁ is substituted or unsubstituted linear —C₃₋₁₂ alkylene;
L₂ is substituted or unsubstituted linear —C₄₋₁₂ alkylene;
L₃ is substituted or unsubstituted linear —C₄₋₁₂ alkylene;
wherein if L₁, L₂, or L₃ is substituted, then L₁, L₂, or L₃ is substituted with 1-5 substituents selected from the group consisting of halogen, deuterium, —CN, linear or branched C₁₋₆ alkyl, C₁₋₂ haloalkyl, —C(═O)R¹⁰, —C(═O)N(R¹⁰)₂, —OR¹⁰, —N(R¹⁰)₂, substituted or unsubstituted C₃₋₆ cycloalkyl, and substituted or unsubstituted 3- to 6-membered heterocycloalkyl; wherein each substituted C₃₋₆ cycloalkyl and substituted 3- to 6-membered heterocycloalkyl is substituted with 1-5 R¹¹;
L₄ is substituted or unsubstituted —C₁₋₂₄ alkylene-; wherein L₄ is optionally substituted with 1 to 10 R¹¹;
X₁ is —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—;
X₂ is —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—;
each R₁, R₂, and R₃, is independently hydrogen, deuterium, halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl;
provided that at least one of R₁, R₂, and R₃ is not hydrogen or deuterium;
or when each R₁, R₂, and R₃ is hydrogen or deuterium, then at least one of L₁, L₂, L₃ is substituted with 1-5 substituents, and at least one of the substituents is not a linear C₁₋₁₀ alkyl substituted at a terminal carbon, or a deuterium;
R₄ is —OH, —OR¹⁰, —O—C(═O)—R¹⁰, —C(═O)—OR¹⁰, —O—C(═O)—OR¹⁰—, —C(═O)—O—C(═O)—R¹⁰, —O—C(═O)—C₀₋₉ alkylene-R¹¹, —(O—C₀₋₉ alkylene-)ₙR¹¹, —O—C(═O)—C₀₋₉ alkylene-N(R¹⁰)₂, —N(R¹⁰)₂, —CH(CH₃)—CH₂—OH, or —C(═O)—C₀₋₉ alkylene-N(R¹⁰)₂;
each R¹⁰ is independently hydrogen, C₁₋₁₀ alkyl, C₁₋₁₀ heteroalkyl, C₁₋₁₀ haloalkyl, C₃₋₁₀ heterocycloalkyl, C₂₋₆ alkenyl, C₂₋₆ alkynyl or C₃₋₁₀ cycloalkyl;
each R¹¹ is independently selected from the group consisting of hydrogen, deuterium, halogen, —CN, —C₁₋₆ alkyl, C₁₋₁₀ heteroalkyl, C₁₋₁₀ haloalkyl, C₃₋₁₀ heterocycloalkyl, C₂₋₆ alkenyl, C₂₋₆ alkynyl, —C(O)OH, —C(O)—O—C₁₋₆ alkyl, —C(O)—C₁₋₆ alkyl, —C(O)NH₂, —C(O)NH(C₁₋₆ alkyl), —C(O)N(C₁₋₆ alkyl)₂, —NH₂, —NH(C₁₋₆ alkyl), —N(C₁₋₆ alkyl)₂, —OH, —O—C₁₋₆ alkyl, —SH, —S(C₁₋₆ alkyl), —S(O)(C₁₋₆ alkyl), —S(O)₂(C₁₋₆ alkyl), or —S(O)₂NH(C₁₋₆ alkyl);
n is an integer from 0-20;
p is 0, 1, 2, 3, or 4; and
q is 0, 1, 2, 3, or 4.

8. The compound of any one of claims 1-78, or its N-oxide, or a pharmaceutically acceptable salt thereof, wherein X₁ is —C(═O)—O—, —O—C(═O)—, —O—C(═O)—O—, —C(═O)—N(R¹⁰)—, or —N(R¹⁰)—C(═O)—.

9. The compound of any one of claims 1-8, or its N-oxide, or a pharmaceutically acceptable salt thereof, wherein X₂ is —C(═O)—O—, —O—C(═O)—, —O—C(═O)—O—, —C(═O)—N(R¹⁰)—, or —N(R¹⁰)—C(═O)—.

10. The compound of any one of claims 1-9, or its N-oxide, or a pharmaceutically acceptable salt thereof, wherein p is 1, 2, 3, or 4; and q is 1, 2, 3, or 4.

11. The compound of any one of claims 1-10, or its N-oxide, or a pharmaceutically acceptable salt thereof, wherein p is 1, 2, or 3; and q is 1, 2, or 3.

12. The compound of any one of claims 1-10, or its N-oxide, or a pharmaceutically acceptable salt thereof, wherein p is 1, and q is 1.

13. The compound of any one of claims 1-10, or its N-oxide, or a pharmaceutically acceptable salt thereof, wherein p is 1, and q is 3.

14. The compound of any one of claims 1-10, or its N-oxide, or a pharmaceutically acceptable salt thereof, wherein p is 3, and q is 1.

15. The compound of any one of claims 1-10, or its N-oxide, or a pharmaceutically acceptable salt thereof, wherein p is 3, and q is 3.

16. The compound of any one of claims 1-10, or its N-oxide, or a pharmaceutically acceptable salt thereof, wherein p is 3, and q is 4.

17. The compound of any one of claims 1-16, having the structure of Formula II: 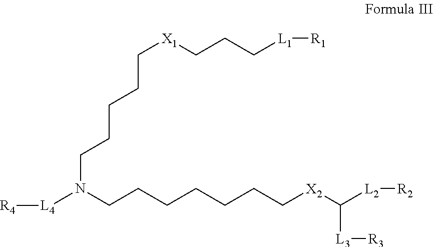
or its N-oxide, or a pharmaceutically acceptable salt thereof.

18. The compound of any one of claims 1-16, having the structure of Formula I-a: 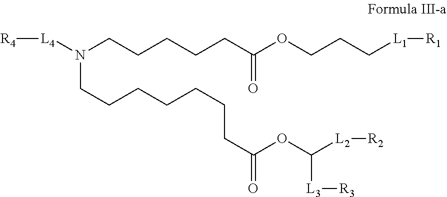
or its N-oxide, or a pharmaceutically acceptable salt thereof.

19. The compound of any one of claims 1-16, having the structure of Formula IV: 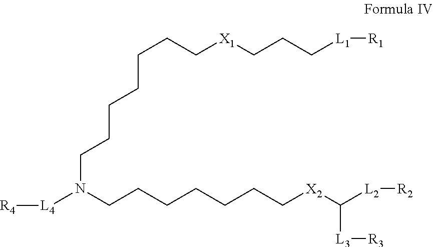
or its N-oxide, or a pharmaceutically acceptable salt thereof.

20. The compound of any one of claims 1-17 and 19, wherein X₁ is —C(═O)—O— or —O—C(═O)—.

21. The compound of any one of claims 1-17 and 19-20, wherein X₂ is —C(═O)—O— or —O—C(═O)—.

22. The compound of any one of claims 1-16, having the structure of Formula IV-a: 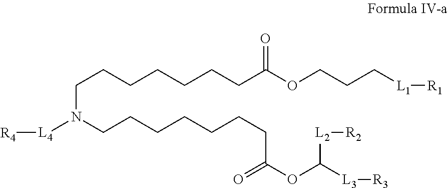
or its N-oxide, or a pharmaceutically acceptable salt thereof.

23. The compound of any one of claims 1-22, or its N-oxide, or a pharmaceutically acceptable salt thereof, wherein at least one of R₁, R₂, and R₃ is not hydrogen or deuterium.

24. The compound of any one of claims 1-23, or its N-oxide, or a pharmaceutically acceptable salt thereof, wherein R₁, R₂, and R₃ are each independently halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl.

25. The compound of any one of claims 1-24, or its N-oxide, or a pharmaceutically acceptable salt thereof, wherein at least one of R₁, R₂, and R₃ is halogen, C₂₋₆ alkenyl, or branched C₃₋₁₀ alkyl.

26. The compound of any one of claims 1-25, or its N-oxide, or a pharmaceutically acceptable salt thereof, wherein at least one of R₁, R₂, and R₃ is fluoro or isopropyl.

27. The compound of any one of claims 1-24, or its N-oxide, or a pharmaceutically acceptable salt thereof, wherein R₁ is halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl.

28. The compound of any one of claims 1-24, or its N-oxide, or a pharmaceutically acceptable salt thereof, wherein R₁ is halogen, —CN, —OR¹⁰, —N(R¹⁰)₂, C₂₋₆ alkenyl, or branched C₃₋₁₀ alkyl.

29. The compound of any one of claims 1-24, or its N-oxide, or a pharmaceutically acceptable salt thereof, wherein R₁ is halogen, —OR¹⁰, C₂₋₆ alkenyl, or branched C₃₋₁₀ alkyl.

30. The compound of any one of claims 1-24, or its N-oxide, or a pharmaceutically acceptable salt thereof, wherein R₁ is halogen, —OH, isopropyl, isobutyl, isopentyl, sec-butyl, tert-butyl, tert-pentyl, or tert-hexyl.

31. The compound of any one of claims 1-24, or its N-oxide, or a pharmaceutically acceptable salt thereof, wherein R₁ is halogen or isopropyl, isobutyl, sec-butyl, or tert-butyl.

32. The compound of any one of claims 1-24, or its N-oxide, or a pharmaceutically acceptable salt thereof, wherein R₂ is halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl.

33. The compound of any one of claims 1-24, or its N-oxide, or a pharmaceutically acceptable salt thereof, wherein R₂ is halogen, —CN, —OR¹⁰, —N(R¹⁰)₂, C₂₋₆ alkenyl, or branched C₃₋₁₀ alkyl.

34. The compound of any one of claims 1-24, or its N-oxide, or a pharmaceutically acceptable salt thereof, wherein R₂ is halogen, —OR¹⁰, C₂₋₆ alkenyl, or branched C₃₋₁₀ alkyl.

35. The compound of any one of claims 1-24, or its N-oxide, or a pharmaceutically acceptable salt thereof, wherein R₂ is halogen, —OH, isopropyl, isobutyl, isopentyl, sec-butyl, tert-butyl, tert-pentyl, or tert-hexyl.

36. The compound of any one of claims 1-24, or its N-oxide, or a pharmaceutically acceptable salt thereof, wherein R₂ is halogen or isopropyl, isobutyl, sec-butyl, or tert-butyl.

37. The compound of any one of claims 1-24, or its N-oxide, or a pharmaceutically acceptable salt thereof, wherein R₃ is halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl.

38. The compound of any one of claims 1-24, or its N-oxide, or a pharmaceutically acceptable salt thereof, wherein R₃ is halogen, —CN, —OR¹⁰, —N(R¹⁰)₂, C₂₋₆ alkenyl, or branched C₃₋₁₀ alkyl.

39. The compound of any one of claims 1-24, or its N-oxide, or a pharmaceutically acceptable salt thereof, wherein R₃ is halogen, —OR¹⁰, C₂₋₆ alkenyl, or branched C₃₋₁₀ alkyl.

40. The compound of any one of claims 1-24, or its N-oxide, or a pharmaceutically acceptable salt thereof, wherein R₃ is halogen, —OH, isopropyl, isobutyl, isopentyl, sec-butyl, tert-butyl, tert-pentyl, or tert-hexyl.

41. The compound of any one of claims 1-24, or its N-oxide, or a pharmaceutically acceptable salt thereof, wherein R₃ is halogen or isopropyl, isobutyl, sec-butyl, or tert-butyl.

42. The compound of any one of claims 1-24, or its N-oxide, or a pharmaceutically acceptable salt thereof, wherein R₃ is halogen, —OR¹⁰, or branched C₃₋₁₀ alkyl; and R₁ and R₂ are hydrogen.

43. The compound of any one of claims 1-24, or its N-oxide, or a pharmaceutically acceptable salt thereof, wherein R₃ is halogen, —OR¹⁰, or branched C₃₋₁₀ alkyl; R₂ is halogen, —OR¹⁰, or branched C₃₋₁₀ alkyl and R₁ is hydrogen.

44. The compound of any one of claims 1-43, or its N-oxide, or a pharmaceutically acceptable salt thereof, wherein each R₁, R₂, and R₃ is hydrogen or deuterium, and at least one of L₁, L₂, L₃ is substituted with 1-5 substituents, and at least one of the substituents is not a linear C₁₋₁₀ alkyl substituted at a terminal carbon of L₁, L₂, L₃, or a deuterium.

45. The compound of any one of claims 1-43, or its N-oxide, or a pharmaceutically acceptable salt thereof, wherein L₁ is substituted or unsubstituted —C₄₋₁₀ alkylene, or —C₃₋₁₂ alkenylalkylene.

46. The compound of claim 45, or its N-oxide, or a pharmaceutically acceptable salt thereof, wherein L₁ is —C₄₋₁₀ alkylene or —C₃₋₁₂ alkenylalkylene, substituted with halogen, linear or branched C₁₋₆ alkyl, or C₁₋₂ haloalkyl.

47. The compound of claim 46, or its N-oxide, or a pharmaceutically acceptable salt thereof, wherein L₁ is —C₄₋₁₀ alkylene or —C₃₋₁₂ alkenylalkylene, substituted with fluoro, methyl, or isopropyl.

48. The compound of any one of claims 1-47, or its N-oxide, or a pharmaceutically acceptable salt thereof, wherein each L₂ and L₃ is independently substituted or unsubstituted —C₄₋₁₀ alkylene, or —C₃₋₁₂ alkenylalkylene.

49. The compound of any one of claims 1-48, or its N-oxide, or a pharmaceutically acceptable salt thereof, wherein L₂ and L₃ are the same.

50. The compound of any one of claims 1-43, or its N-oxide, or a pharmaceutically acceptable salt thereof, wherein L₂ and L₃ are different.

51. The compound of any one of claims 1-47, or its N-oxide, or a pharmaceutically acceptable salt thereof, each L₂ and L₃ is independently substituted —C₄₋₁₀ alkylene, or —C₃₋₁₂ alkenylalkylene, wherein the substituent is halogen, or linear or branched C₁₋₆ alkyl.

52. The compound of any one of claims 1-47, or its N-oxide, or a pharmaceutically acceptable salt thereof, each L₂ and L₃ is independently a substituted —C₄₋₁₀ alkylene or —C₃₋₁₂ alkenylalkylene, wherein —C₄₋₁₀ alkylene or —C₃₋₁₂ alkenylalkylene is substituted with fluoro, methyl, or isopropyl.

53. The compound of any one of claims 1-52, or its N-oxide, or a pharmaceutically acceptable salt thereof, wherein L₄ is substituted or unsubstituted —C₁₋₁₂ alkylene, or —C₃₋₂₄ alkenylalkylene; wherein L₄ is optionally substituted with 1 to 10 R¹¹.

54. The compound any one of claims 1-52, or its N-oxide, or a pharmaceutically acceptable salt thereof, wherein L₄ is substituted or unsubstituted —C₁₋₆ alkylene, or —C₃₋₁₂ alkenylalkylene; wherein L₄ is optionally substituted with 1 to 10 R¹¹.

55. The compound of any one of claims 1-52, or its N-oxide, or a pharmaceutically acceptable salt thereof, wherein L₄ is —CH₂—, —CH₂—CH₂—, —CH₂—CH₂—CH₂—, —CH₂—(CH₂)₂—CH₂—, —CH₂—(CH₂)₃—CH₂—, or —CH₂—(CH₂)₄—CH₂—.

56. The compound of any one of claims 1-52, or its N-oxide, or a pharmaceutically acceptable salt thereof, wherein L₄ is —CH₂—, —CH₂—CH₂—, or —CH₂—CH₂—CH₂—.

57. The compound of any one of claims 1-56, or its N-oxide, or a pharmaceutically acceptable salt thereof, wherein R₄ is —OH, —OR¹⁰, —O—C(═O)—R¹⁰, —C(═O)—OR¹⁰, —O—C(═O)—C₀₋₉ alkylene-N(R¹¹)₂, —O—C(═O)—C₀₋₉ alkylene-R¹¹, —(O—C₀₋₉ alkylene-)ₙR¹¹, —CH(CH₃)—CH₂—OH, or —C(═O)—C₀₋₉ alkylene-N(R¹¹)₂.

58. The compound of any one of claims 1-56, or its N-oxide, or a pharmaceutically acceptable salt thereof, wherein R₄ is —OH, —CH(CH₃)—CH₂—OH, or —O—C(═O)—(CH₂)₃—N(Me)₂.

59. A compound, or its N-oxide, or a pharmaceutically acceptable salt thereof, having the structure listed in Table 1.

60. A compound, or its N-oxide, or a pharmaceutically acceptable salt thereof, having the structure listed in Table 2.

61. A compound, or its N-oxide, or a pharmaceutically acceptable salt thereof, having the structure:

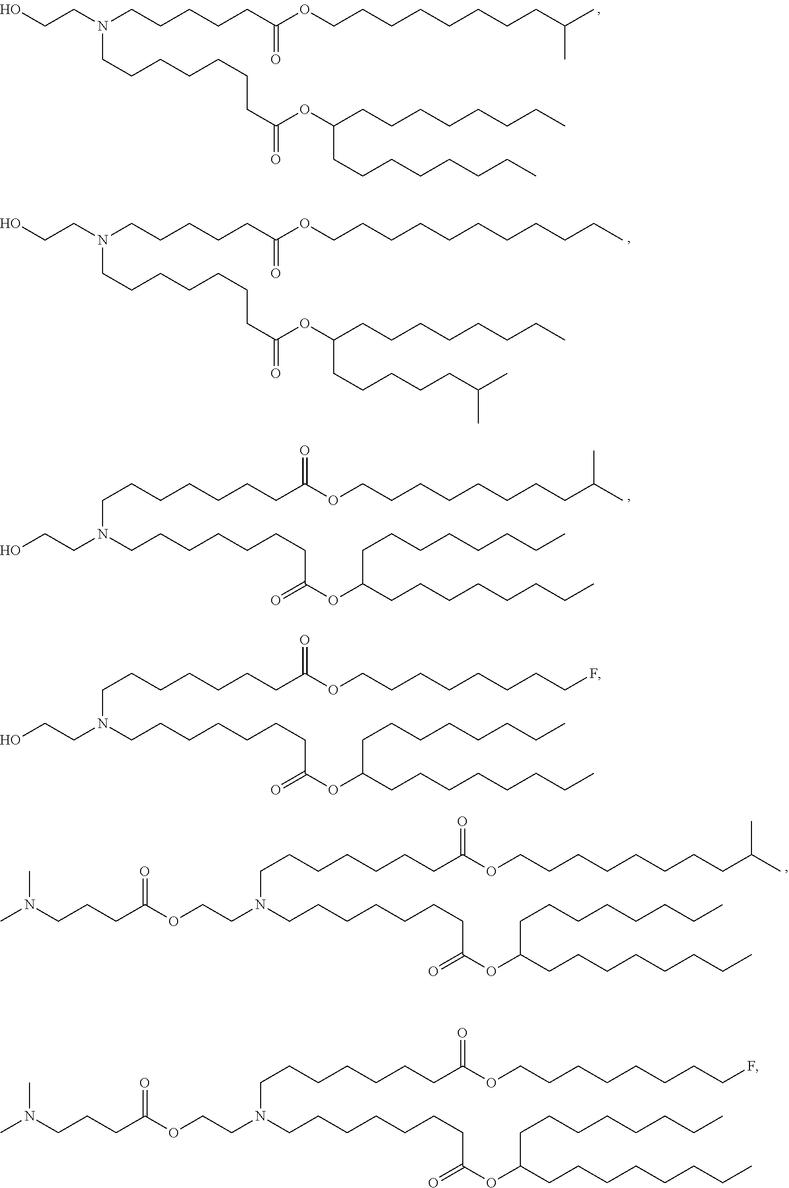

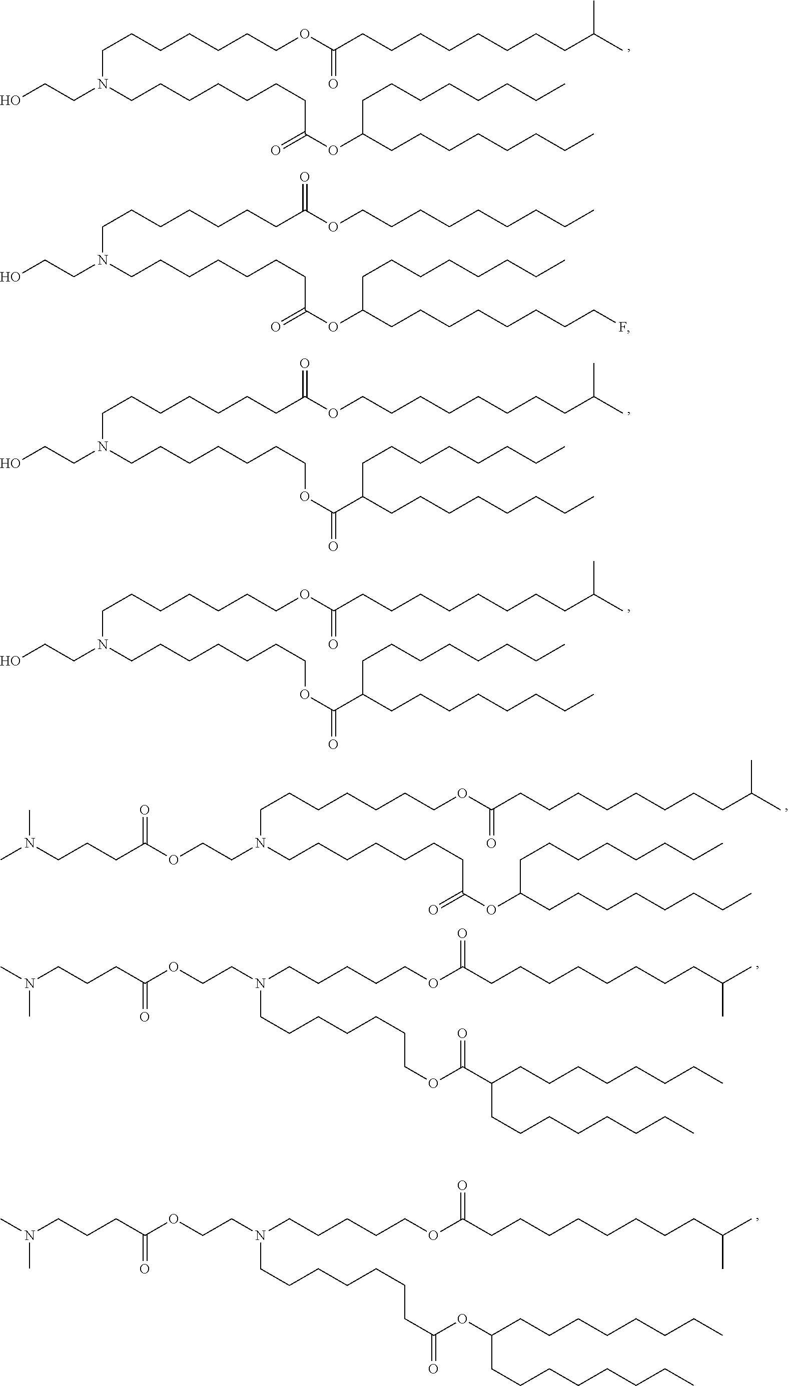

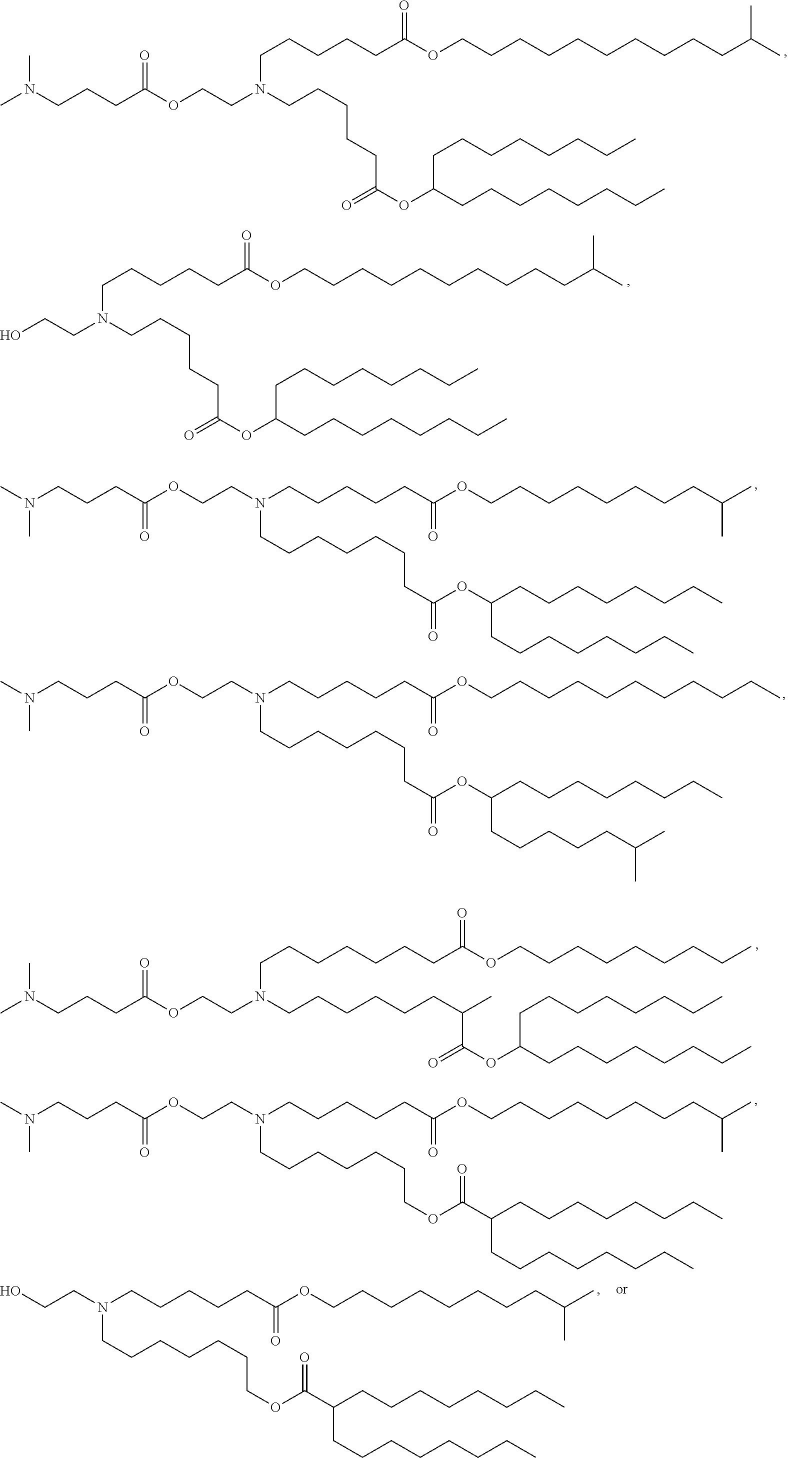

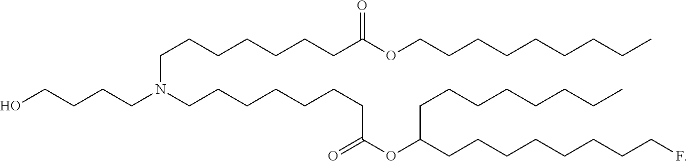 [SMILES: CCCCCCCCCOC(=O)CCCCCCCN(CCCCO)CCCCCCCC(=O)OC(CCCCCCCC)CCCCCCCCF]

62. A compound, or its N-oxide, or a pharmaceutically acceptable salt thereof, wherein the compound is:

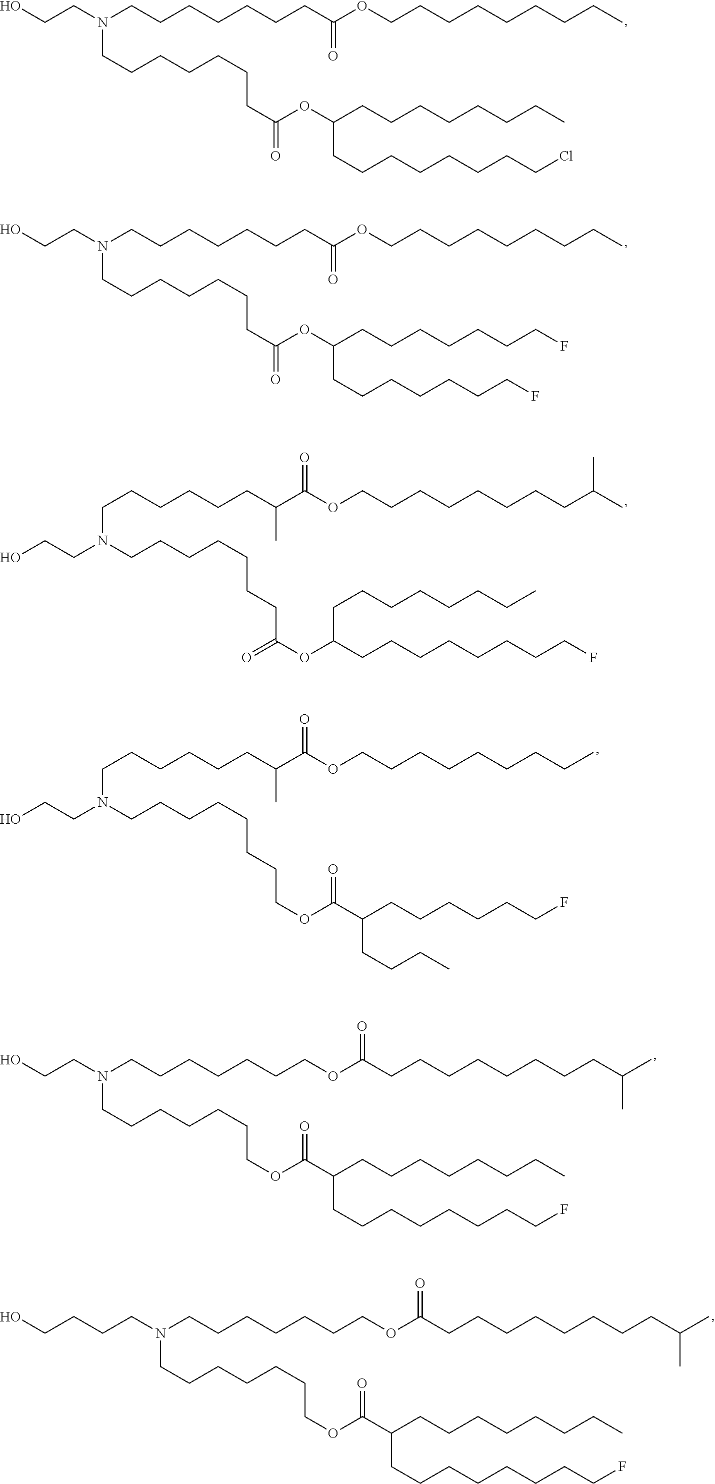 [SMILES: CCCCCCCCCOC(=O)CCCCCCCN(CCO)CCCCCCCC(=O)OC(CCCCCCCC)CCCCCCCCCl ; CCCCCCCCCOC(=O)CCCCCCCN(CCO)CCCCCCCC(=O)OC(CCCCCCCF)CCCCCCCF ; CCCCCCCCC(CCCCCCCCF)OC(=O)CCCCCCCN(CCO)CCCCCCC(C)C(=O)OCCCCCCCCC(C)C ; CCCCCCCCCOC(=O)C(C)CCCCCCN(CCO)CCCCCCCCOC(=O)C(CCCC)CCCCCCF ; CCCCCCCCC(CCCCCCCCF)C(=O)OCCCCCCCN(CCO)CCCCCCCOC(=O)CCCCCCCCC(C)C ; CCCCCCCCC(CCCCCCCCF)C(=O)OCCCCCCCN(CCCCO)CCCCCCCOC(=O)CCCCCCCCC(C)C]

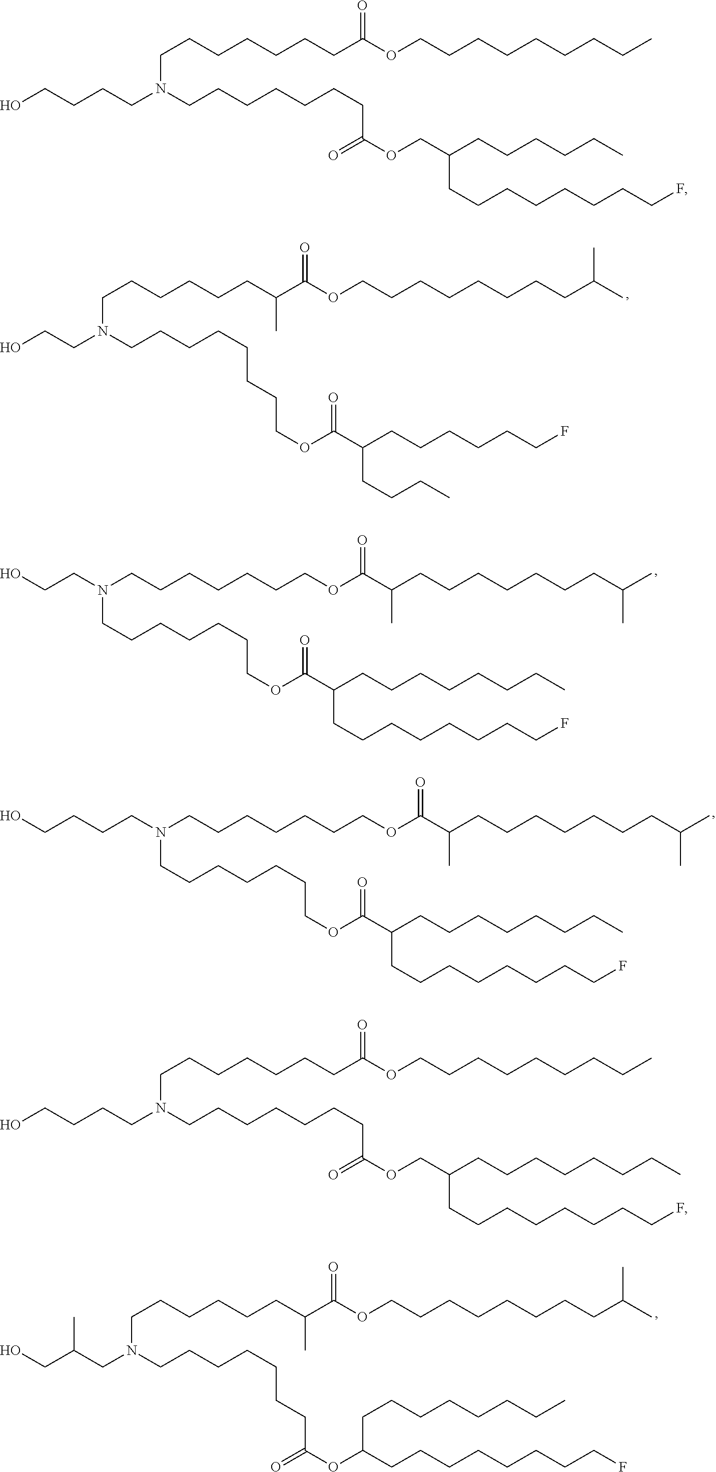

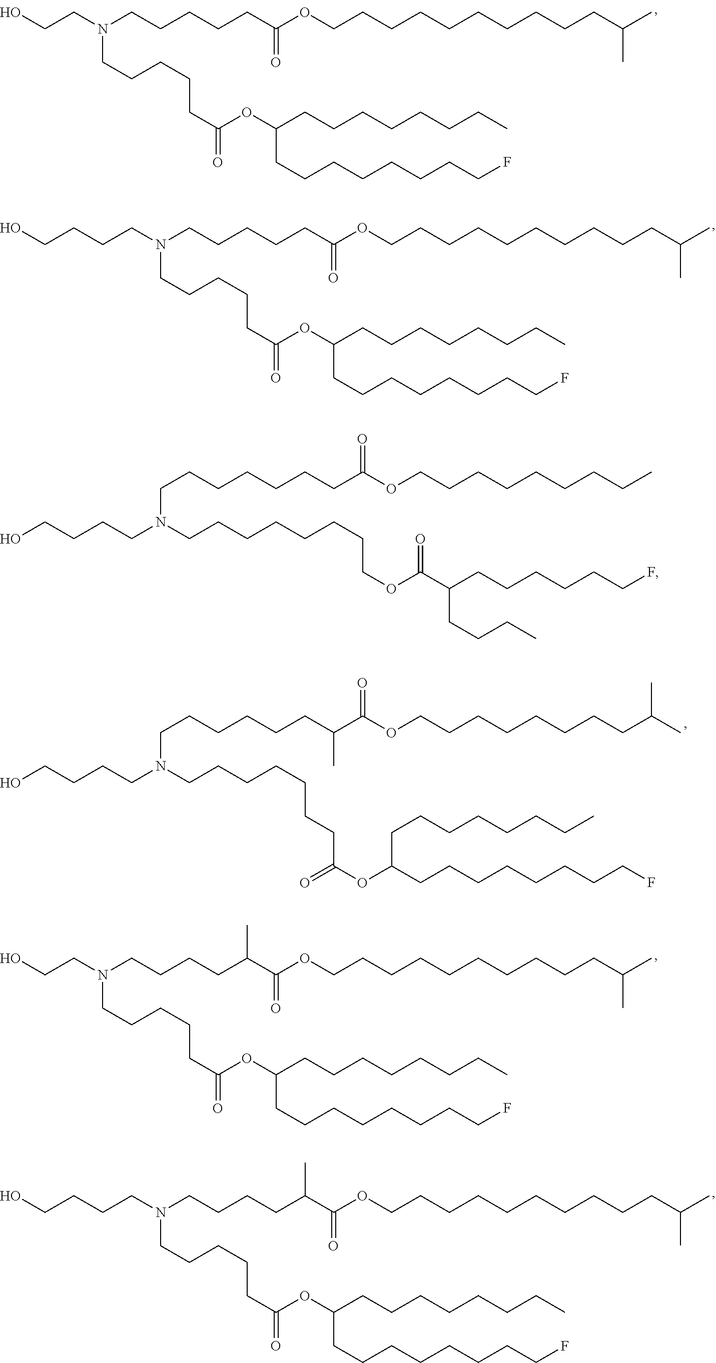

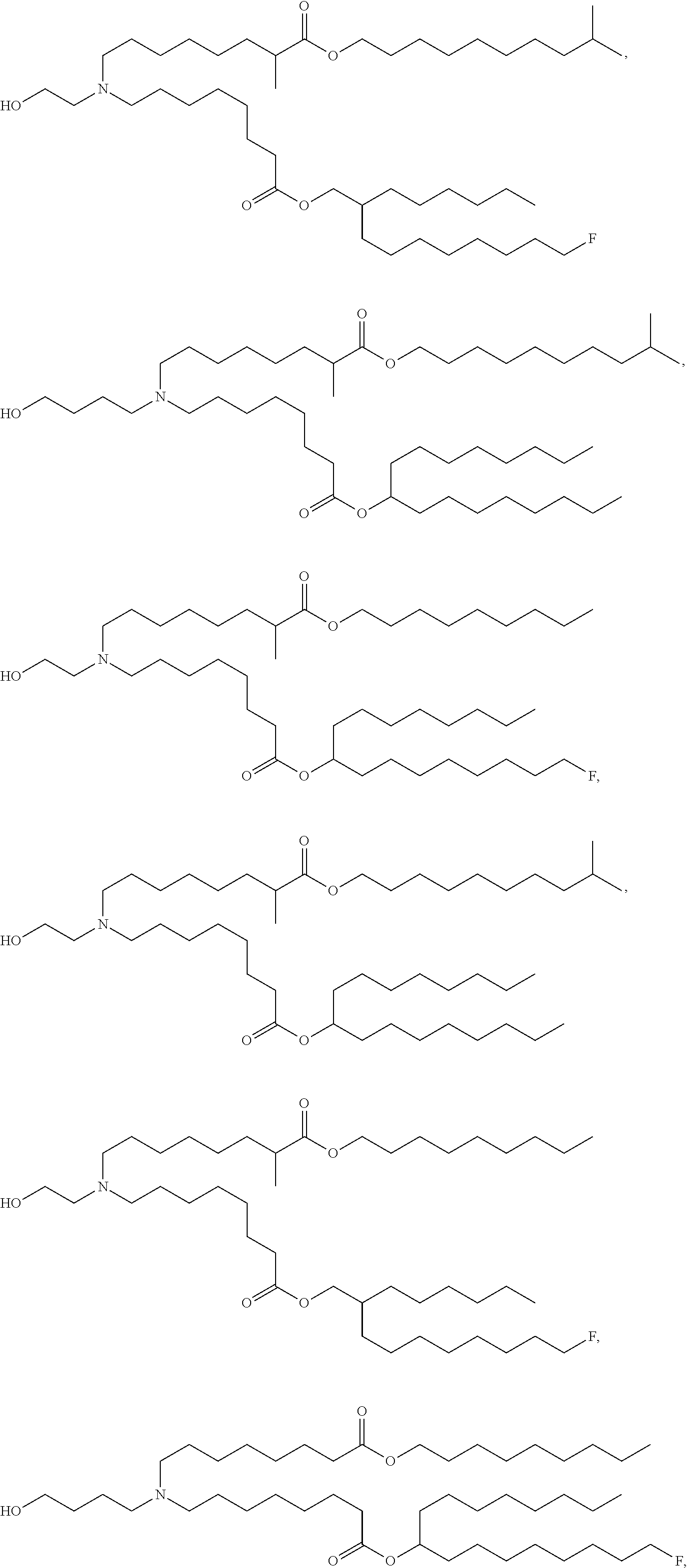 [SMILES: CCCCCCC(CCCCCCCCF)COC(=O)CCCCCCCN(CCO)CCCCCCC(C)C(=O)OCCCCCCCCC(C)C ; CCCCCCCCC(CCCCCCCC)OC(=O)CCCCCCCN(CCCCO)CCCCCCC(C)C(=O)OCCCCCCCCC(C)C ; CCCCCCCCCOC(=O)C(C)CCCCCCN(CCO)CCCCCCCC(=O)OC(CCCCCCCC)CCCCCCCCF ; CCCCCCCCC(CCCCCCCC)OC(=O)CCCCCCCN(CCO)CCCCCCC(C)C(=O)OCCCCCCCCC(C)C ; CCCCCCCCCOC(=O)C(C)CCCCCCN(CCO)CCCCCCCC(=O)OCC(CCCCCC)CCCCCCCCF ; CCCCCCCCCOC(=O)CCCCCCCN(CCCCO)CCCCCCCC(=O)OC(CCCCCCCC)CCCCCCCCF]

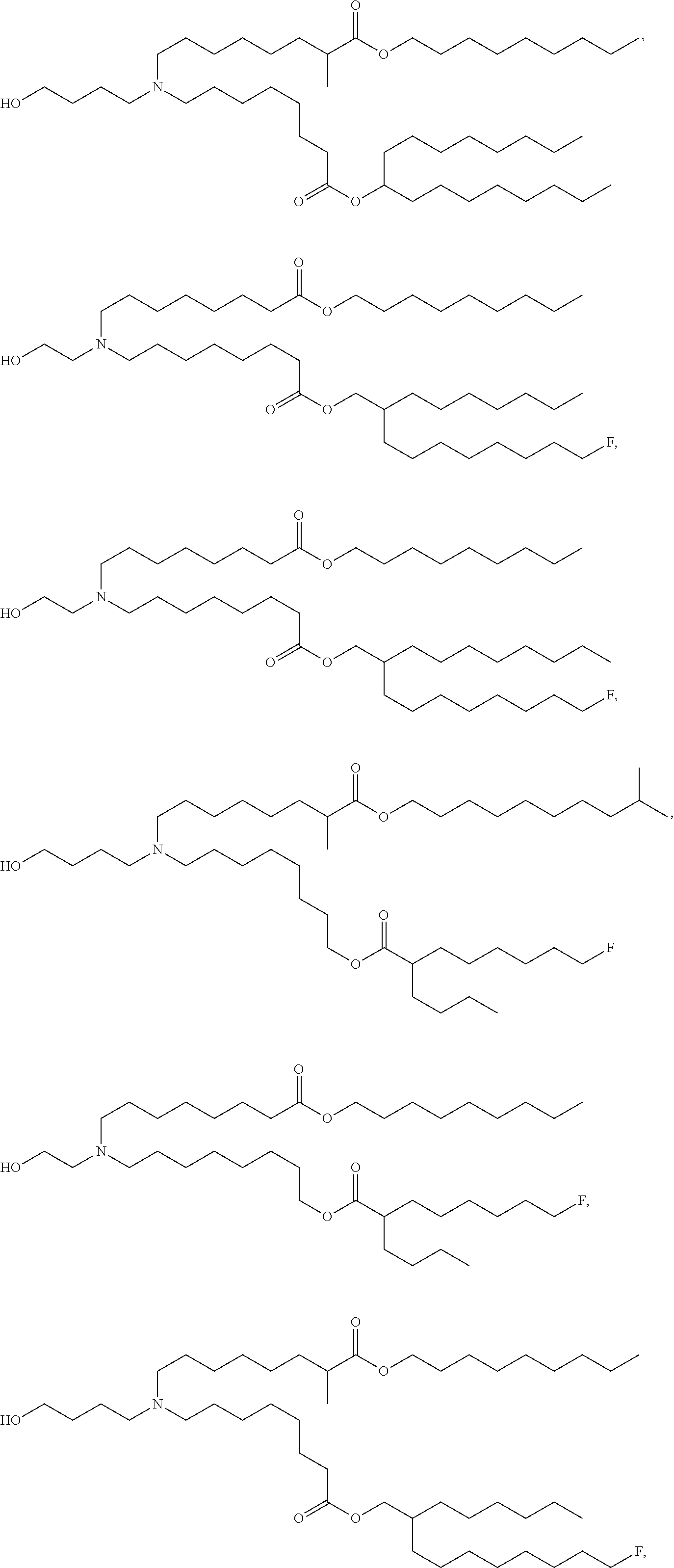 [SMILES: CCCCCCCCCOC(=O)C(C)CCCCCCN(CCCCO)CCCCCCCC(=O)OC(CCCCCCCC)CCCCCCCC ; CCCCCCCCCOC(=O)CCCCCCCN(CCO)CCCCCCCC(=O)OCC(CCCCCCC)CCCCCCCCF ; CCCCCCCCCOC(=O)CCCCCCCN(CCO)CCCCCCCC(=O)OCC(CCCCCCCC)CCCCCCCCF ; CCCCC(CCCCCCF)C(=O)OCCCCCCCCN(CCCCO)CCCCCCC(C)C(=O)OCCCCCCCCC(C)C ; CCCCCCCCCOC(=O)CCCCCCCN(CCO)CCCCCCCCOC(=O)C(CCCC)CCCCCCF ; CCCCCCCCCOC(=O)C(C)CCCCCCN(CCCCO)CCCCCCCC(=O)OCC(CCCCCC)CCCCCCCCF]

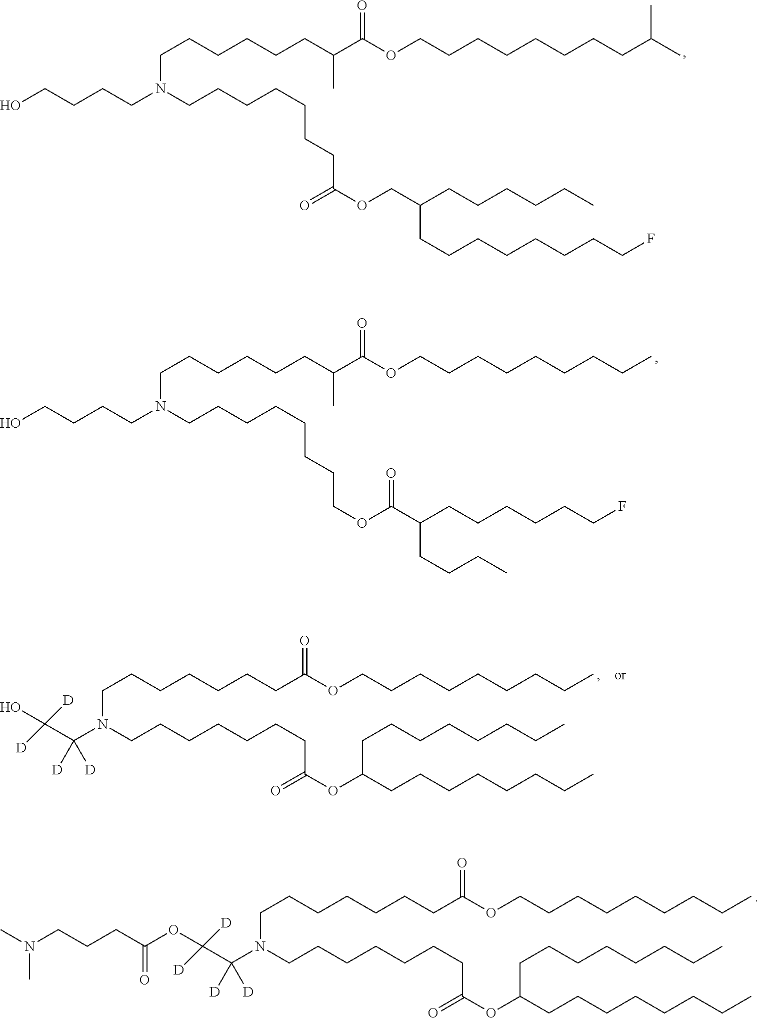

63. A compound of Formula VI, or an N-oxide, or a pharmaceutically acceptable salt thereof: 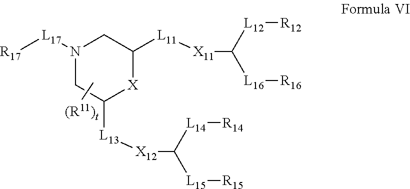
wherein:
X is O, S, or C(R¹¹)₂;
L₁₁ is substituted or unsubstituted linear —C₃₋₁₂ alkylene, wherein one or more methylene units of the group are optionally and independently replaced with —CR¹¹═CR¹¹—;
L₁₂ is substituted or unsubstituted linear —C₃₋₁₂ alkylene, wherein one or more methylene units of the group are optionally and independently replaced with —CR¹¹═CR¹¹—;
L₁₃ is substituted or unsubstituted linear —C₃₋₁₂ alkylene, wherein one or more methylene units of the group are optionally and independently replaced with —CR¹¹═CR¹¹—;
L₁₄ is substituted or unsubstituted linear —C₃₋₁₂ alkylene, wherein one or more methylene units of the group are optionally and independently replaced with —CR¹¹═CR¹¹—;
L₁₅ is substituted or unsubstituted linear —C₀₋₁₂ alkylene, wherein one or more methylene units of the group are optionally and independently replaced with —CR¹¹═CR¹¹;
L₁₆ is substituted or unsubstituted linear —C₀₋₁₂ alkylene, wherein one or more methylene units of the group are optionally and independently replaced with —CR¹¹═CR¹¹—;
wherein if L₁₁, L₁₂, L₁₃, L₁₄, L₁₅, or L₁₆ is substituted, then L₁₁, L₁₂, L₁₃, L₁₄, L₁₅, or L₁₆ is substituted with 1-5 substituents selected from the group consisting of halogen, deuterium, —CN, linear or branched C₁₋₆ alkyl, C₁₋₂ haloalkyl, —C(═O)R¹⁰, —C(═O)N(R¹⁰)₂, —OR¹⁰, —N(R¹⁰)₂, substituted or unsubstituted C₃₋₆ cycloalkyl, and substituted or unsubstituted 3- to 6-membered heterocycloalkyl; wherein each substituted C₃₋₆ cycloalkyl and substituted 3- to 6-membered heterocycloalkyl is substituted with 1-5 R¹¹;
X₁₁ is a bond, —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—;
X₁₂ is a bond, —C(═O)—O—, —O—C(═O)—, —O—C(═O)—O—, —C(═O)—N(R¹⁰)—, —N(R¹⁰)—C(═O)—, S—S, —C(═O)—S—, or —C(C═S)—O—;
each R₁₂, R₁₄, R₁₅ and R₁₆, is independently hydrogen, deuterium, halogen, —CN, —OR¹⁰, —N(R¹⁰)₂, branched C₃₋₁₀ alkyl, C₃₋₁₀ cycloalkyl, C₂₋₆ alkenyl, or C₂₋₆ alkynyl;
L₁₇ is substituted or unsubstituted —C₁₋₂₄ alkylene-; wherein L₁₇ is optionally substituted with 1 to 10 R¹¹;
R¹⁷ is —OH, —OR¹⁰, —O—C(═O)—R¹⁰, —C(═O)—OR¹⁰, —O—C(═O)—OR¹⁰—, —C(═O)—O—C(═O)—R¹⁰, —O—C(═O)—C₀₋₉ alkylene-R¹¹, —(O—C₀₋₉ alkylene-)ₙR¹¹, —O—C(═O)—C₀₋₉ alkylene-N(R¹⁰)₂, —N(R¹⁰)₂, —CH(CH₃)—CH₂—OH, or —C(═O)—C₀₋₉ alkylene-N(R¹⁰)₂;
each R¹⁰ is independently hydrogen, C₁₋₁₀ alkyl, C₁₋₁₀ heteroalkyl, C₁₋₁₀ haloalkyl, C₃₋₁₀ heterocycloalkyl, C₂₋₆ alkenyl, C₂₋₆ alkynyl or C₃₋₁₀ cycloalkyl;
each R¹¹ is independently selected from the group consisting of hydrogen, deuterium, halogen, —CN, —C₁₋₆ alkyl, C₁₋₁₀ heteroalkyl, C₁₋₁₀ haloalkyl, C₃₋₁₀ heterocycloalkyl, C₂₋₆ alkenyl, C₂₋₆ alkynyl, —C(O)OH, —C(O)—O—C₁₋₆ alkyl, —C(O)—C₁₋₆ alkyl, —C(O)NH₂, —C(O)NH(C₁₋₆ alkyl), —C(O)N(C₁₋₆ alkyl)₂, —NH₂, —NH(C₁₋₆ alkyl), —N(C₁₋₆ alkyl)₂, —OH, —O—C₁₋₆ alkyl, —SH, —S(C₁₋₆ alkyl), —S(O)(C₁₋₆ alkyl), —S(O)₂(C₁₋₆ alkyl), or —S(O)₂NH(C₁₋₆ alkyl); and
t is 0, 1, 2, 3, or 4.

64. The compound of claim 63, or its N-oxide, or a pharmaceutically acceptable salt thereof, wherein X is O.

65. The compound of claim 63 or 64, or its N-oxide, or a pharmaceutically acceptable salt thereof, wherein X₁₁ is a bond, —C(═O)—O— or —O—C(═O)—.

66. The compound of any one of claims 63-65, or its N-oxide, or a pharmaceutically acceptable salt thereof, wherein X₁₂ is a bond, —C(═O)—O— or —O—C(═O)—.

67. The compound of any one of claims 63-66, or its N-oxide, or a pharmaceutically acceptable salt thereof, wherein L₁₆ is C₀ alkylene and R₁₆ is hydrogen.

68. The compound of any one of claims 63-67, or its N-oxide, or a pharmaceutically acceptable salt thereof, wherein Lis is C₀ alkylene and R₁₅ is hydrogen.

69. A compound, or its N-oxide, or a pharmaceutically acceptable salt thereof, having the structure listed in Table 3.

70. A nanoparticle composition comprising a lipid component, wherein the lipid component comprises a compound of any one of claims 1-69.

71. The nanoparticle composition of claim 70, wherein the lipid component further comprises a structural lipid.

72. The nanoparticle composition of claim 71, wherein the structural lipid comprises cholesterol, fecosterol, sitosterol, ergosterol, campesterol, stigmasterol, brassicasterol, tomatidine, ursolic acid, or alpha-tocopherol.

73. The nanoparticle composition of any one of claims 70-72, wherein the lipid component further comprises a PEG lipid.

74. The nanoparticle composition of claim 73, wherein the PEG lipid is a PEG-modified phosphatidy-lethanolamine, a PEG-modified phosphatidic acid, a PEG-modified ceramide, a PEG-modified dialkylamine, a PEG-modified diacylglycerol, or a PEG-modified dialkylglycerol.

75. The nanoparticle composition of any one of claims 70-74, wherein the lipid component further comprises a cationic and/or ionizable lipid.

76. The nanoparticle composition of claim 75, wherein the cationic and/or ionizable lipid is 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), or (2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)).

77. The nanoparticle composition of any one of claims 70-76, wherein the lipid component further comprises a phospholipid, a structural lipid, and a PEG lipid.

78. The nanoparticle composition of any one of claims 70-77, wherein the lipid component comprises about 30% to about 60% of the compounds of claims 1-55, about 0% to about 30% phospholipid, about 20% to about 50% structural lipid, and about 0% to about 10% PEG lipid.

79. The nanoparticle composition of any one of claims 70-78, further comprising a therapeutic and/or prophylactic agent.

80. The nanoparticle composition of claim 79, wherein the therapeutic and/or prophylactic agent is nucleic acid.

81. The nanoparticle composition of any one of claims 79-80, wherein the therapeutic and/or prophylactic agent is ribonucleic acid (RNA).

82. The nanoparticle composition of claim 81, wherein the RNA is a small interfering RNA (siRNA), an asymmetrical interfering RNA (aiRNA), a microRNA (miRNA), a Dicer-substrate RNA (dsRNA), a small hairpin RNA (shRNA), or a messenger RNA (mRNA).

83. The nanoparticle composition of any one of claims 81-82, wherein the RNA is mRNA.

84. The nanoparticle composition of claim 83, wherein the mRNA comprises at least one nucleic acid modification.

85. The nanoparticle composition of claim 84, wherein the at least one nucleic acid modification comprises a N1-methylpseudouridine (m1ψ).

86. A pharmaceutical composition comprising the nanoparticle composition of any one of claims 70-85, and a pharmaceutically acceptable carrier.

87. A method of delivering a therapeutic agent to a subject in need thereof, the method comprising administering to the subject a nanoparticle composition of any one of claims 70-85, or a pharmaceutical composition of claim 86, thereby the therapeutic agent is delivered to the subject.

88. A method of producing a polypeptide of interest in a cell, the method comprising contacting the cell with a nanoparticle composition of any one of claims 70-85, or a pharmaceutical composition of claim 86, wherein the therapeutic agent is an mRNA, wherein the mRNA encodes the polypeptide of interest.

89. The method of claim 88, wherein the mRNA is capable of being translated in the cell, thereby producing the polypeptide of interest.

90. A method of selectively delivering a therapeutic agent to a mammalian organ, the method comprising administering to a mammal a nanoparticle composition of any one of claims 70-85, or a pharmaceutical composition of claim 86.

91. The method of claim 90, wherein administering the nanoparticle composition or pharmaceutical composition comprises contacting the mammalian organ with the nanoparticle composition, thereby delivering the therapeutic agent to the organ.

92. The method of claim 91, wherein the therapeutic agent is delivered to the organ.

93. A method of treating a disease or disorder in a subject in need thereof, the method comprising administering to the subject a therapeutically effective amount of a nanoparticle composition of any one of claims 70-85, or a pharmaceutical composition of claim 86.

94. The method of claim 93, wherein the disease or disorder is cancer.
